(19) United States
(12) Patent Application Publication
(10) Pub. No.: US 2023/0083303 A1
(43) Pub. Date: Mar. 16, 2023

(54) POLYCYCLIC COMPOUND AND AN ORGANIC ELECTROLUMINESCENCE DEVICE COMPRISING THE POLYCYCLIC COMPOUND OR THE COMPOSITION

(71) Applicant: IDEMITSU KOSAN CO.,LTD., Chiyoda-ku (JP)

(72) Inventors: Thomas SCHAEFER, Liestal (BL) (CH); Peter MURER, Oberwil (BL) (CH); Annemarie WOLLEB, Fehren (CH); Carsten ROTHE, Dresden (DE); Yuichi NISHIMAE, Basel (CH)

(73) Assignee: IDEMITSU KOSAN CO.,LTD., Chiyoda-ku (JP)

(21) Appl. No.: 17/606,235

(22) PCT Filed: Apr. 27, 2020
(86) PCT No.: PCT/IB2020/053931

§ 371 Date: Oct. 25, 2021

(30) Foreign Application Priority Data
Apr. 26, 2019 (EP) 19171408.8

(51) Int. Cl. C07F 5/02; C07D 307/91; C07C 15/28; C07C 15/30; C07C 15/38; C07C 15/20; C07D 493/04; C07D 405/10; C07D 409/10; C07D 407/10; C07D 333/76; C07D 403/14; C07D 235/08; C07C 211/54; C07C 255/51; H01L 51/00

(52) U.S. Cl. CPC C07F 5/027; C07D 307/91; C07C 15/28; C07C 15/30; C07C 15/38; C07C 15/20; C07D 493/04; C07D 405/10; C07D 409/10; C07D 407/10; C07D 333/76; C07D 403/14; C07D 235/08; C07C 211/54; C07C 255/51; H01L 51/0062; H01L 51/0052; H01L 51/0054; H01L 51/0055; H01L 51/0056; H01L 51/0059; H01L 51/0072; H01L 51/0073; H01L 51/0074; H01L 51/0067; H01L 51/008; H01L 51/5096

(57) ABSTRACT

Specific polycyclic compounds, a material for an organic electroluminescence device comprising said specific polycyclic compound, an organic electroluminescence device comprising said specific polycyclic compound, an electronic equipment comprising said organic electroluminescence device, a process for preparing said polycyclic compounds, and the use of said polycyclic compounds in an organic electroluminescence. (Formula I) (I)

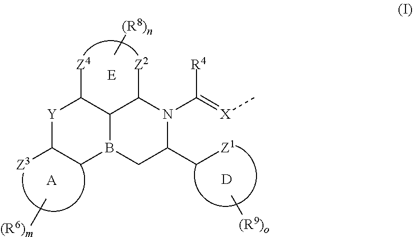

The present invention relates to specific polycyclic compounds, a material for an organic electroluminescence device comprising said specific polycyclic compound, an organic electroluminescence device comprising said specific polycyclic compound, an electronic equipment comprising said organic electroluminescence device, a process for preparing said polycyclic compounds, and the use of said polycyclic compounds in an organic electroluminescence.

When a voltage is applied to an organic electroluminescence device (hereinafter may be referred to as an organic EL device), holes are injected to an emitting layer from an anode and electrons are injected to an emitting layer from a cathode. In the emitting layer, injected holes and electrons are re-combined and excitons are formed.

An organic EL device comprises an emitting layer between the anode and the cathode. Further, there may be a case where it has a stacked layer structure comprising an organic layer such as a hole-injecting layer, a hole-transporting layer, an electron-injecting layer, an electron-transporting layer, etc.

The applicability of π-conjugated boron compounds is known in the art.

An issue of such π-conjugated boron compounds is low stability. Boron, which is a group 13 element, is an electron-deficient element having an empty p-orbital and is thus susceptible to attack by nucleophilic species. Accordingly, boron containing compounds are generally unstable.

There have been reported various methods for improving the thermodynamic stability of π-conjugated boron compounds.

US 2019/0013478 A1 relates to an organic electroluminescent element comprising an anode, a cathode, and at least one organic layer sandwiched between the anode and the cathode, wherein at least one of the organic layer comprises a π-conjugated boron compound represented by the following general formula 1:

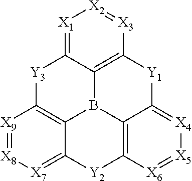

wherein:

X₁ to X₉ each independently represents —CW or a nitrogen atom,

W represents a hydrogen atom or a substituent, and

Y₁ to Y₃ each independently represents an oxygen atom or a sulfur atom.

The π-conjugated boron compound according to US 2019/0013478 A1, which has a bipolar ability and can comply with various energy levels, can be used as a fluorescent compound, luminescent host, or assist dopant and also as a compound suitable for hole transport and electron transport.

US 2018/0069182 A1 relates to a polycyclic aromatic compound represented by the following general formula (I), or a dimer of a polycyclic aromatic compound having two structures each represented by the following general formula (I). US 2018/0069182 A1 preferably relates to a polycyclic aromatic compound represented by the following general formula (2), or a dimer of a polycyclic aromatic compound having two structures each represented by the following general formula (2).

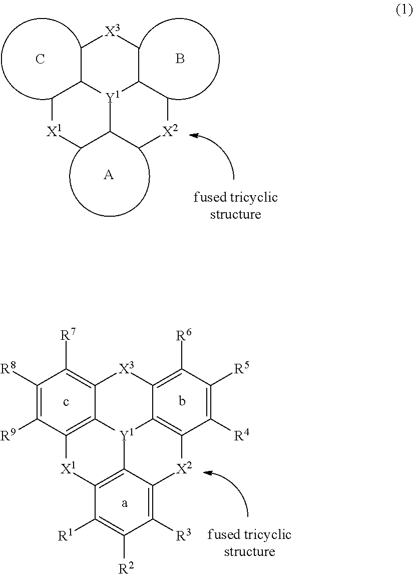

Y¹ represents B, P, P—O, P—S, P(—R)₂, Al, Ga, As, Si—R, Ge—R, Sn—R, Sb, Sb—O, Sb—S, Sb(—R)₂, Sb to which orthochloranil is bonded, Bi, Bi—O, Bi—S, Bi(—R)₂, or Bi to which orthochloranil is bonded, R of the moieties P(—R)₂, Si—R, Ge—R, Sn—R, Sb(—R)₂, and Bi(—R)₂ represents an aryl, an alkyl, an alkoxy, an aryloxy, or a halogen atom, and two Rs among the moieties P(—R)₂, Sb(—R)2 and Bi(—R)₂ may be bonded to each other via a single bond or by fusing to form a ring,

X¹, X², and X³ each independently represents O, N—R, S, or Se, R of the moiety N—R represents an aryl which may be substituted, a heteroaryl which may be substituted, an alkyl, or a cycloalkyl, at least one of X¹, X², and X³ represents N—R, and R of the moiety N—R may be bonded to the ring A, ring B, and/or ring C via a linking group or a single bond or by fusing.

The polycyclic aromatic compound according to US 2018/0069182 A1 and a dimer thereof can be used as a material for an organic device. Examples of the organic device include an organic electroluminescent element, an organic field effect transistor, and an organic thin film solar cell.

In the compounds disclosed in the documents mentioned above, all three aryl groups attached to the boron atom are bridged.

WO 2018/203666 A1 relates to a compound represented by the following general formula (I),

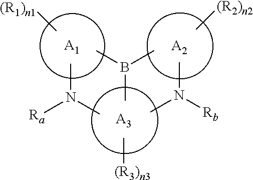

wherein

Rₐ, R_{b}, R₁ to R₃ each independently represent hydrogen; heavy hydrogen; halogen; cyano; nitro; substituted or unsubstituted silyl; substituted or unsubstituted amino; substituted or unsubstituted C₁₋₆₀ alkyl; substituted or unsubstituted C₁₋₆₀ haloalkyl; substituted or unsubstituted C₁₋₆₀ alkoxy; substituted or unsubstituted C₁₋₆₀ haloalkoxy; substituted or unsubstituted C₃₋₆₀ cycloalkyl; substituted or unsubstituted C₂₋₆₀ alkenyl; substituted or unsubstituted C₆₋₆₀ aryl; substituted or unsubstituted C₆₋₆₀ aryloxy; or substituted or unsubstituted C₂₋₆₀ heteroaryl containing at least one heteroatom selected from the group consisting of N, O and S,

provided that at least one of Rₐ, R_{b} and R₁ to R₃ is a substituted or unsubstituted silyl group and Rₐ can be linked to the A₁ or A₃ ring by a single bond, —O—, —S—, —C(Q₁)(Q₂)— or —N(Q₃)—, R_{b} can be linked to the A₂ or A₃ ring by a single bond, —O—, —S—, —C(Q₄)(Q₅)—, or the A₁ and the A₂ ring can be linked to each other by —N(Q₆)—, —S—, —C(Q₇)(Q₈)—, or —N(Q₉)—, wherein Q₁ to Q₉ are each independently hydrogen; heavy hydrogen; C₁₋₁₀ alkyl; or n₁ to n₃ is an integer of 0 to 10.

Therefore, in the compounds of WO 2018/203666 A1, a substituted or unsubstituted silyl group is mandatory.

EP 3 109 253 A1 relates to a polycyclic aromatic compound in which plural aromatic rings are linked via boron atoms, oxygen atoms and the like, the production thereof, and to a material for organic EL element containing a polycyclic aromatic compound. The polycyclic aromatic compound is represented by the following general formula (I):

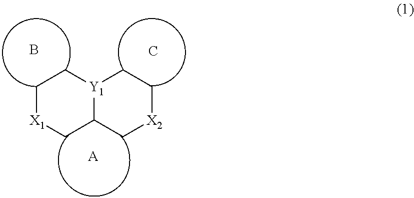

wherein in formula (I),

ring A, ring B and ring C each independently represent an aryl ring or a heteroaryl ring, while at least one hydrogen atom in these rings may be substituted;

Y¹ represents B, P, P═O, P═S, Al, Ga, As, Si—R or Ge—R, wherein R of the moieties Si—R and Ge—R represents an aryl or an alkyl;

X¹ and X² each independently represent 0, N—R, S or Se, wherein R of the moiety N—R represents an aryl which may be substituted, a heteroaryl which may be substituted, or an alkyl which may be substituted, and R of the moiety N—R may be bonded to the ring A, ring B and/or ring C by a linking group or a single bond; and

at least one hydrogen atom in the compound or structure represented by formula (I) may be substituted by a halogen atom or a deuterium atom.

CN 107 501 311 A relates to an organic electroluminescent material selected from the compounds represented by the general formula (I)

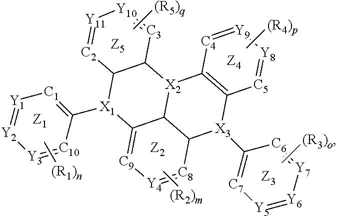

wherein X₁, X₂, and X₃ each independently represents a nitrogen atom or a boron atom, at least one of X₁, X₂, and X₃ is a boron atom and at least one of X₁, X₂, and X₃ is a nitrogen atom;

Y₁, Y₂, Y₃, Y₄, Y₅, Y₆, Y₇, Y₈, Y₉, Y₁₀, and Y₁₁ e ach independently represents a carbon atom, a nitrogen atom, a silicon atom, a phosphorus atom, an oxygen atom, or a sulfur atom;

R₁, R₂, R₃, R₄, and R₅ each independently represents a hydrogen atom, a deuterium atom, an electron-withdrawing group, or an electron-donating group; the electron-withdrawing group includes a deuterated electron-withdrawing group, and the electron-donating group includes deuterated electron-donor groups;

at least one pair of carbon atoms in C₁ and C₂, C₃ and C₄, C₅ and C₆, C₇ and C₈, and C₉ and C₁₀ are connected via an alkylene group or an alkenylene group to form a 5- to 7-membered ring;

the ring represented by Z₁, Z₂, Z₃, Z₄, Z₅ is fused or unfused with C₆₋₁₈ aryl, C₃₋₁₈ heteroaryl; m, n, o, p, and q each independently represents an integer of 1 to 4.

CN 109 575 059 A relates to a thermally activated delayed fluorescent material having the following structure:

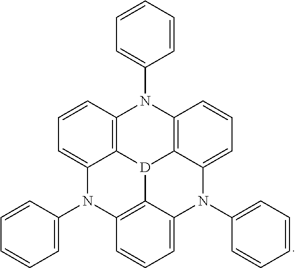

One example for a suitable compound according to CN 109 575 059 A is shown in the following:

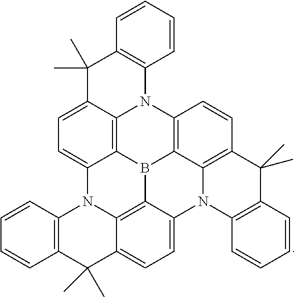 [SMILES: CC1(C)c2ccccc2N2c3ccc4c5c3B3c6c(ccc1c62)N1c2ccccc2C(C)(C)c2ccc(c3c21)N5c1ccccc1C4(C)C]

CN 107 417 715 A relates to an organic electroluminescent material selected from at least one of the compounds of the general formula (I) and the general formula (II)

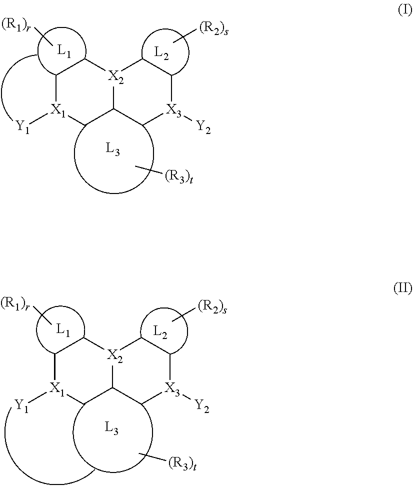

wherein X₁, X₂ and X₃ each independently represents a nitrogen atom or a boron atom; at least one of X₁, X₂ and X₃ is a nitrogen atom and at least one of X₁, X₂ and X₃ is a boron atom;

L₁, L₂, L₃ each independently represents an aromatic ring, a heteroaromatic ring or a condensed ring;

Y₁ represents a substituted or unsubstituted C₆ to C₄₈ aryl group or a substituted or unsubstituted C₃ to C₄₈ heteroaryl group;

Y₂ represents a substituted or unsubstituted amino group, a substituted or unsubstituted C₁ to C₃₆ alkyl group, a substituted or unsubstituted C₆ to C₄₈ aryl group, a substituted or unsubstituted C₃ to C₄₈ heteroaryl group,

R₁, R₂ and R₃ are each independently selected from the group consisting of a hydrogen atom, a deuterium atom, a cyano group, a substituted or unsubstituted C₁ to C₃₆ alkyl group, a substituted or unsubstituted C₁ to C₃₆ alkoxy group, a substituted or unsubstituted C₆ to C₄₈ aryl, or a substituted or unsubstituted C₃ to C₄₈ heteroaryl,

the alkyl group includes a deuterated alkyl group, the alkoxy group includes a deuterated alkoxy group, the aryl group includes a deuterated aryl group, and the heteroaryl group includes a deuterated heteroaryl group;

the substituent is at least one selected from the group consisting of C₁ to C₁₂ alkyl, C₁ to C₁₂ deuteroalkyl, C₆ to C₁₂ aryl, C₆ to C₁₂ deuterated aryl, C₅ to C₁ heteroaryl, C₅ to C₁ deuterated heteroaryl;

r, s, t are each independently an integer selected from 1 to 4.

CA 3 016 789 A1 relates to an organic compound, in particular for use in optoelectronic devices, having a structure of formula I

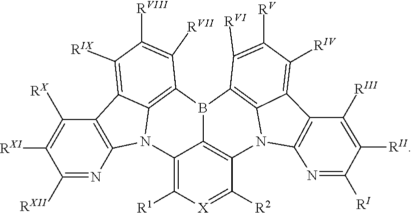

wherein X is N or CR₃; R₁, R₂, R₃, RI, RII, RIII, RIV, RV, RVI, RVII, RVIII, RIX, RX, RXI, and RXII is independently from each other selected from the group consisting of: hydrogen, deuterium, which is optionally substituted with one or more substituents R₄; C₁-C₄₀-alkoxy, which is optionally substituted with one or more substituents R₄; C₂-C₄₀-alkenyl, which is optionally substituted with one or more substituents R₄; C₂-C₄₀-alkynyl, which is optionally substituted with one or more substituents R₄; C₆-C₆₀-aryl, which is optionally substituted with one or more substituents R₄; C₃-C₅₇-heteroaryl, which is optionally substituted with one or more substituents R₄; CN; CF₃; N(R₄)₂; OR₄, and Si(R₄)₃; wherein substituent pairs selected from the group consisting of RIX and RVIII, RVIII and RVII, RVI and RV, and RV and RIV optionally form a mono- or polycyclic, aliphatic, aromatic and/or benzo-fused ring system with each other.

The compounds of the last four references mentioned above are characterized by a (hetero)aromatic substitution of the N atoms (if present) in the following structural element:

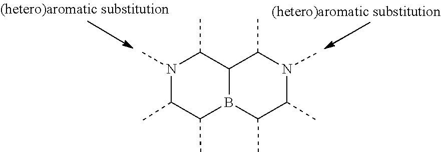

However, the specific structure and substitution pattern of polycyclic compounds has a significant impact on the performance of the polycyclic compounds in organic electronic devices.

Therefore, notwithstanding the developments described above, there remains a need for organic electroluminescence devices comprising new materials, especially dopant (=emitter) materials, to provide improved performance of electroluminescence devices.

Accordingly, it is an object of the present invention, with respect to the aforementioned related art, to provide an organic electroluminescence device having a high luminous efficiency and a novel compound that can be used as a material for an organic electroluminescence device having a long lifetime and/or low driving voltage. More particularly, it should be possible to provide dopant (=emitter) materials, especially blue light emitting dopant materials for use in organic electroluminescence devices.

Furthermore, the materials should be suitable for providing organic electroluminescence devices which ensure good performance of the organic electroluminescence devices, especially a long lifetime and/or low driving voltage.

Said object is according to one aspect of the present invention solved by a polycyclic compound represented by formula (I):

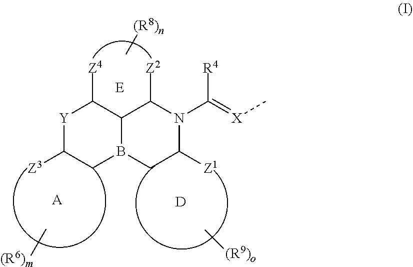

wherein

ring A, ring E and ring D each independently represents an aromatic group having 6 to 30 ring carbon atoms or a heteroaromatic group having 3 to 30 ring atoms;

X represents CR⁵ or N;

[symbol] the dotted line represents a single bond connected with Z¹ or connected with Z²;

Z¹ represents C in the case that it is connected with the dotted line at X, and Z¹ represents CR^{X9} or N in the case that it is not connected with the dotted line at X;

Z² represents C in the case that it is connected with the dotted line at X, and Z² represents CR^{X8} or N in the case that it is not connected with the dotted line at X;

Y represents NR¹, O, S,

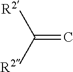

or CR²₂;

R⁴ and R⁵ each independently represents H, halogen, a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, a substituted or unsubstituted aryloxy group having 6 to 24 ring carbon atoms, a substituted or unsubstituted alkylthio group having 1 to 25 carbon atoms, a substituted or unsubstituted arylthio group having 6 to 24 ring carbon atoms, an alkyl and/or aryl substituted silyl group, an alkyl or aryl substituted carbonyl group, an alkyl, aryl or heteroaryl substituted amino group, an alkyl or aryl substituted amide group, an alkyl or aryl substituted carboxyl group, a substituted phosphoryl group, CN, or a substituted or unsubstituted fluoroalkyl group having 1 to 25 carbon atoms; or

R⁴ and R⁵ may form together an unsubstituted or substituted aliphatic ring;

R¹ represents a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, or a substituted or unsubstituted fluoroalkyl group having 1 to 25 carbon atoms; or a group of formula

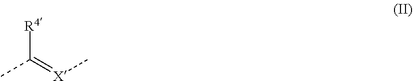

wherein X′ represents CR^{5′} or N; R^{4′}, R^{5′} and X′ are defined as R⁴, R⁵ and X;

with the difference that R⁴ and R^{5′} may form together an unsubstituted or substituted ring;

[symbol] the dotted line at X′ represents a single bond connected with Z³ or connected with Z⁴;
Z³ represents C in the case that it is connected with the dotted line at X′, and Z³ represents CR^{X6A} or N in the case that it is not connected with the dotted line at X′;
Z⁴ represents C in the case that it is connected with the dotted line at X′, and Z⁴ represents CR^{X8A} or N in the case that it is not connected with the dotted line at X′;

[symbol] the other dotted line in the group of formula (II) represents a bonding site to the N atom of the group NR¹;

wherein R¹ may be connected to ring A or ring E;

R², R^{2′} and R^{2″} each independently represents H, halogen, a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, a substituted or unsubstituted aryloxy group having 6 to 24 ring carbon atoms, a substituted or unsubstituted alkylthio group having 1 to 25 carbon atoms, a substituted or unsubstituted arylthio group having 6 to 24 ring carbon atoms, an alkyl and/or aryl substituted silyl group, an alkyl or aryl substituted carbonyl group, an alkyl, aryl or heteroaryl substituted amino group, an alkyl or aryl substituted amide group, an alkyl or aryl substituted carboxyl group, a substituted phosphoryl group, CN, or a substituted or unsubstituted fluoroalkyl group having 1 to 25 carbon atoms, or

one of the residues R^{2′} or R^{2″} may be connected with ring A or ring E; R⁶, R⁸, R⁹, R^{X6A}, R^{X8A}, R^{X8} and R^{X9}, each independently represents H, halogen, a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, a substituted or unsubstituted aryloxy group having 6 to 24 ring carbon atoms, a substituted or unsubstituted alkylthio group having 1 to 25 carbon atoms, a substituted or unsubstituted arylthio group having 6 to 24 ring carbon atoms, an alkyl and/or aryl substituted silyl group, an alkyl or aryl substituted carbonyl group, an alkyl or aryl substituted carboxyl group, alkyl or aryl substituted amide group, an alkyl, aryl or heteroaryl substituted amino group, a substituted phosphoryl group, CN, or a substituted or unsubstituted fluoroalkyl group having 1 to 25 carbon atoms;

wherein two adjacent groups R⁶, two adjacent groups R⁸ and/or two adjacent groups R⁹ and/or R^{X6A} and a group R⁶ adjacent to R^{X6A} and/or R^{X9} and a group R⁹ adjacent to R^{X9} and/or R^{X8} and a group R⁸ adjacent to R^{X8} and/or R^{X8A} and a group R⁸ adjacent to R^{X8A}, may form together an unsubstituted or substituted ring;

n is 0 or 1; and

m and o are each independently 0, 1, 2 or 3.

According to one aspect of the present invention, a material for an organic electroluminescence device, comprising at least one compound of formula (I) is provided.

The term organic EL device (organic electroluminescence device) is used interchangeably with the term organic light-emitting diode (OLED) in the present application.

According to another aspect of the invention, the following organic electroluminescence device is provided: An organic electroluminescence device comprising a cathode, an anode, and one or more organic thin film layers comprising an emitting layer disposed between the cathode and the anode, wherein at least one layer of the organic thin film layers comprises at least one compound of formula (I).

According to another aspect of the invention, an emitting layer of the organic electroluminescence device is provided which comprises least one compound of formula (I).

According to another aspect of the invention, an emitting layer of the organic electroluminescence device is provided which comprises least one compound of formula (I) as a dopant material and an anthracene compound as a host material.

According to another aspect of the invention, an electronic equipment provided with the organic electroluminescence device according to the present invention is provided.

According to another aspect of the invention, a process for preparing a compound of formula (I) according to the present invention is provided.

According to another aspect of the invention, the use of a compound of formula (I) according to the present invention in an organic electroluminescence device is provided.

The specific polycyclic compounds of the present invention according to formula (I) comprising an indolo or an imidazole group may be used as a material that is highly suitable in organic electroluminescence devices.

The compounds of formula (I) according to the present invention are especially characterized by the following feature:

R⁴ and R⁵ may form together an unsubstituted or substituted aliphatic ring, but not a (hetero)aromatic ring.

The compounds of formula (I) can in principal be used in any layer of an EL device. Preferably, the compound of formula (I) is a dopant (=emitter) in organic EL elements, especially in the light-emitting layer, more preferably a fluorescent dopant. Particularly, the compounds of formula (I) are used as fluorescent dopants in organic EL devices, especially in the light-emitting layer.

It has been found by the inventors that the specific compounds of formula (I) show a narrow emission characteristic, preferably a narrow fluorescence, more preferably a narrow blue fluorescence. Such a narrow emission characteristic is suitable to prevent energy losses by outcoupling. The compounds of formula (I) according to the present invention preferably have a Full width at half maximum (FWHM) of lower than 50 nm, more preferably lower than 40 nm, even more preferably lower than 35 nm, most preferably lower than 30 nm. Further most preferably from lower than 28 nm.

It has further been found that organic EL devices comprising the compounds of the present invention are generally characterized by long lifetimes.

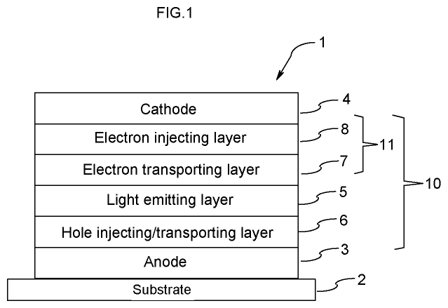

FIG. 1 is a view showing a schematic configuration of one embodiment of the organic EL device of the invention.

The terms halogen, a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, a substituted or unsubstituted aralkyl group having 6 to 30 ring carbon atoms, a substituted or unsubstituted heteroaryl group having 3 to 18 ring atoms, a substituted or unsubstituted heterocyclic group having 3 to 18 ring atoms, a substituted or unsubstituted aryloxy group having 6 to 24 ring carbon atoms, a substituted or unsubstituted alkylthio group having 1 to 25 carbon atoms, a substituted or unsubstituted arylthio group having 6 to 24 ring carbon atoms, an alkyl and/or aryl substituted silyl group, an alkyl or aryl substituted carbonyl group, an alkyl, aryl or heteroaryl substituted amino group, an alkyl or aryl substituted amide group, an alkyl or aryl substituted carboxyl group, a substituted phosphoryl group, CN, and a substituted or unsubstituted fluoroalkyl group having 1 to 25 carbon atoms, are known in the art and generally have the following meaning, if said groups are not further specified in specific embodiments mentioned below:

The substituted or unsubstituted aryl group having 6 to 30 ring carbon atoms, preferably 6 to 24 ring carbon atoms, more preferably 6 to 18 ring carbon atoms, may be a non-condensed aryl group or a condensed aryl group. Specific examples thereof include phenyl group, naphthyl group, phenanthryl group, biphenyl group, terphenyl group, quaterphenyl group, fluoranthenyl group, triphenylenyl group, phenanthrenyl group, fluorenyl group, anthracenyl, chrysenyl, spirofluorenyl group, 9,9-diphenylfluorenyl group, 9,9-spirobi[9H-fluorene]-2-yl group, 9,9-dimethylfluorenyl group, benzo[c]phenanthrenyl group, benzo[a]triphenylenyl group, naphtho[1,2-c]phenanthrenyl group, naphtho[1,2-a]triphenylenyl group, dibenzo[a,c]triphenylenyl group, benzo[a]fluoranthenyl group, benzo[j]fluoranthenyl group, benzo[k]fluoranthenyl group and benzo[b]fluoranthenyl group, with phenyl group, naphthyl group, biphenyl group, terphenyl group, phenanthryl group, triphenylenyl group, fluorenyl group, spirobifluorenyl group, and fluoranthenyl group being preferred, and phenyl group, 1-naphthyl group, 2-naphthyl group, biphenyl-2-yl group, biphenyl-3-yl group, biphenyl-4-yl group, phenanthrene-9-yl group, phenanthrene-3-yl group, phenanthrene-2-yl group, triphenylene-2-yl group, 9,9-dimethylfluorene-2-yl group, fluoranthene-3-yl group, fluoranthene-2-yl group, fluoranthene-8-yl group being more preferred.

The heteroaryl group having 3 to 18 ring atoms may be a non-condensed heteroaryl group or a condensed heteroaryl group. Specific examples thereof include the residues of pyrrole ring, isoindole ring, benzofuran ring, isobenzofuran ring, benzothiophene, dibenzothiophene ring, isoquinoline ring, quinoxaline ring, quinazoline, phenanthridine ring, phenanthroline ring, pyridine ring, pyrazine ring, pyrimidine ring, pyridazine ring, indole ring, quinoline ring, acridine ring, carbazole ring, furan ring, thiophene ring, benzoxazole ring, benzothiazole ring, benzimidazole ring, dibenzofuran ring, triazine ring, oxazole ring, oxadiazole ring, thiazole ring, thiadiazole ring, triazole ring, imidazole ring, 4-imidazo[1,2-a]benzimidazoyl, 5-benzimidazo[1,2-a]benzimidazoyl, and benzimidazolo[2,1-b][1,3]benzothiazolyl, with the residues of dibenzofuran ring, carbazole ring, and dibenzothiophene ring being preferred, and the residues of dibenzofuran-1-yl group, dibenzofuran-3-yl group, dibenzofuran-2-yl group, dibenzofuran-4-yl group, 9-phenylcarbazole-3-yl group, 9-phenylcarbazole-2-yl group, 9-phenylcarbazole-4-yl group, dibenzothiophene-2-yl group, and dibenzothiophene-4-yl, dibenzothiophene-1-yl group, and dibenzothiophene-3-yl group being more preferred.

The heterocyclic group having a ring structure formed of 3 to 30 atoms (heterocyclic group having 3 to 30 ring atoms), preferably 5 to 18 ring atoms, may be a non-condensed heterocyclic group or a condensed heterocyclic group. Specific examples and preferred examples are the same groups as mentioned above concerning the heteroaryl group having 3 to 18 ring atoms.

Examples of the alkyl group having 1 to 25 carbon atoms include methyl group, ethyl group, n-propyl group, isopropyl group, n-butyl group, s-butyl group, isobutyl group, t-butyl group, n-pentyl group, n-hexyl group, n-heptyl group, n-octyl group, n-nonyl group, n-decyl group, n-undecyl group, n-dodecyl group, n-tridecyl group, n-tetradecyl group, n-pentadecyl group, n-hexadecyl group, n-heptadecyl group, n-octadecyl group, neopentyl group, 1-methylpentyl group, with methyl group, ethyl group, n-propyl group, isopropyl group, n-butyl group, s-butyl group, isobutyl group, t-butyl group being preferred. Preferred are alkyl groups having 1 to 8 carbon atoms. Suitable examples for alkyl groups having 1 to 8 carbon atoms are mentioned before.

Examples of the alkenyl group having 2 to 25 carbon atoms include those disclosed as alkyl groups having 2 to 25 carbon atoms but comprising at least one double bond, preferably one, or where possible, two or three double bonds.

Examples of the alkynyl group having 2 to 25 carbon atoms include those disclosed as alkyl groups having 2 to 25 carbon atoms but comprising at least one triple bond, preferably one, or where possible, two or three triple bonds.

Examples of the cycloalkyl group having 3 to 25 ring carbon atoms include cyclopropyl group, cyclobutyl group, cyclopentyl group, cyclohexyl group, cyclooctyl group, and adamantyl group, with cyclopentyl group, and cyclohexyl group being preferred. Preferred are cycloalkyl groups having 3 to 6 carbon atoms. Suitable examples for cycloalkyl groups having 3 to 6 carbon atoms are mentioned before.

Examples of alkyl and/or aryl substituted silyl groups including alkylsilyl groups having 1 to 10 carbon atoms, preferably 1 to 5 carbon atoms, including trimethylsilyl group, triethylsilyl group, tributylsilyl group, dimethylethylsilyl group, t-butyldimethylsilyl group, propyldimethylsilyl group, dimethylisopropylsilyl group, dimethylpropylsilyl group, dimethylbutylsilyl group, dimethyltertiarybutylsilyl group, diethylisopropylsilyl group, and arylsilyl groups having 6 to 30 carbon atoms, preferably 6 to 18 carbon atoms, including phenyldimethylsilyl group, diphenylmethylsilyl group, diphenyltertiarybutylsilyl group, and triphenylsilyl group, with diphenyltertiarybutylsilyl group and t-butyldimethylsilyl group being preferred.

Examples of halogen atoms include fluorine, chlorine, bromine, and iodine, with fluorine being preferred.

Examples of an alkoxy group having 1 to 25 carbon atoms, preferably 1 to 8 carbon atoms, include those having an alkyl portion selected from the alkyl groups mentioned above.

Examples of an aryloxy group having 6 to 24 ring carbon atoms include those having an aryl portion selected from the aromatic hydrocarbon groups mentioned above.

Examples of an alkylthio group having 1 to 25 carbon atoms include those having an alkyl portion selected from the alkyl groups mentioned above.

Examples of an arylthio group having 6 to 24 ring carbon atoms include those having an aryl portion selected from the aromatic hydrocarbon groups mentioned above.

Examples of substituted phosphoryl groups are di-substituted phosphoryl groups having a substituent selected from the group consisting of a substituted or unsubstituted alkyl group having 1 to 25 carbon atoms and a substituted or unsubstituted aromatic hydrocarbon group having 6 to 24 ring carbon atoms. A preferred phosphoryl group is a diphenylphosphin oxide group.

Examples of alkyl or aryl substituted carbonyl groups include those having an alkyl portion selected from the alkyl groups mentioned above and/or having an aryl portion selected from the aromatic hydrocarbon groups mentioned above.

Examples of a fluoroalkyl group having 1 to 25 carbon atoms include the alkyl groups mentioned above wherein the hydrogen atoms thereof are partly or entirely substituted by fluor atoms.

Examples of an alkylamino group (alkyl substituted amino group), preferably an alkylamino group having 1 to 25 ring carbon atoms include those having an alkyl portion selected from the alkyl groups mentioned above.

Examples of an arylamino group (aryl substituted amino group), preferably an arylamino group having 6 to 24 ring carbon atoms include those having an aryl portion selected from the aromatic hydrocarbon groups mentioned above.

Examples of a heteroarylamino group (heteroaryl substituted amino group), preferably a heteroarylamino group having 3 to 18 ring atoms include those having an aryl portion selected from the aromatic hydrocarbon groups mentioned above.

Examples of the optional aralkyl group having 6 to 30 ring carbon atoms include benzyl group, 2-phenylpropane-2-yl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, α-naphthylmethyl group, 1-β-naphthylethyl group, 2-β-naphthylethyl group, 1-β-naphthylisopropyl group, 2-β-naphthylisopropyl group, 1-pyrrolylmethyl group, 2-(1-pyrrolyl)ethyl group, p-methylbenzyl group, m-methylbenzyl group, o-methylbenzyl group, p-chlorobenzyl group, m-chlorobenzyl group, o-chlorobenzyl group, p-bromobenzyl group, m-bromobenzyl group, o-bromobenzyl group, p-iodobenzyl group, m-iodobenzyl group, o-iodobenzyl group, p-hydroxybenzyl group, m-hydroxybenzyl group, o-hydroxybenzyl group, p-aminobenzyl group, m-aminobenzyl group, o-aminobenzyl group, p-nitrobenzyl group, m-nitrobenzyl group, o-nitrobenzyl group, p-cyanobenzyl group, m-cyanobenzyl group, o-cyanobenzyl group, 1-hydroxy-2-phenylisopropyl group, and 1-chloro-2-phenylisopropyl group.

Examples of a carboxyalkyl group (alkyl substituted carboxyl group), preferably a carboxyalkyl group having 1 to 25 carbon atoms, preferably 1 to 5 carbon atoms, include those having an alkyl portion selected from the alkyl groups mentioned above.

Examples of a carboxyaryl group (aryl substituted carboxyl group), preferably a carboxyaryl group having 6 to 24 carbon atoms, preferably 6 to 18 carbon atoms, include those having an aryl portion selected from the aromatic hydrocarbon groups mentioned above.

Examples of a carboxamidalkyl group (alkyl substituted amide group), preferably a carboxamidalkyl group having 1 to 25 carbon atoms, preferably 1 to 5 carbon atoms include those having an alkyl portion selected from the alkyl groups mentioned above.

Examples of a carboxamidaryl group (aryl substituted amide group), preferably a carboxamidaryl group having 6 to 24 carbon atoms, preferably 6 to 18 carbon atoms, include those having an aryl portion selected from the aromatic hydrocarbon groups mentioned above.

Examples of the optional substituent(s) indicated by “substituted or unsubstituted” and “may be substituted” referred to above or hereinafter include a halogen atom (fluorine, chlorine, bromine, iodine), a cyano group, an alkyl group having 1 to 25, preferably 1 to 6 carbon atoms, a cycloalkyl group having 3 to 25, preferably 5 to 12 carbon atoms, an alkoxy group having 1 to 25, preferably 1 to 5 carbon atoms, a fluoroalkyl group having 1 to 25, preferably 1 to 5 carbon atoms, an alkylamino group having 1 to 25 carbon atoms, preferably 1 to 5 carbon atoms, a carboxyalkyl group having 1 to 25 carbon atoms, preferably 1 to 5 carbon atoms, a carboxamidalkyl group having 1 to 25 carbon atoms, preferably 1 to 5 carbon atoms, a silyl group, an aryl group having 6 to 30 ring carbon atoms, preferably 6 to 18 ring carbon atoms, an aryloxy group having 6 to 24, preferably 6 to 18 ring carbon atoms, an alkylthio group having 1 to 25, preferably 1 to 5 carbon atoms, an arylthio group having 6 to 24, preferably 6 to 18 ring carbon atoms, an arylamino group having 6 to 24 carbon atoms, preferably 6 to 18 carbon atoms, a carboxyaryl group having 6 to 24 carbon atoms, preferably 6 to 18 carbon atoms, a carboxamidaryl group having 6 to 24 carbon atoms, preferably 6 to 18 carbon atoms, an heteroaryl group having 3 to 18 ring atoms, preferably 5 to 14 ring atoms, and an heterocyclic group having 3 to 18 ring atoms, preferably 5 to 14 ring atoms.

The optional substituent is preferably a fluorine atom, a cyano group, an alkyl group having 1 to 25 carbon atoms, an aryl group having 6 to 30 ring carbon atoms, preferably 6 to 18 ring carbon atoms, and a heteroaryl group having 3 to 18 ring atoms, preferably 5 to 14 ring atoms; more preferably a cyano group, a phenyl group, a naphthyl group, a biphenyl group, a terphenyl group, a phenanthryl group, a triphenylenyl group, a fluorenyl group, a spirobifluorenyl group, a fluoranthenyl group, a residue based on a dibenzofuran ring, a residue based on a carbazole ring, and a residue based on a dibenzothiophene ring, a methyl group, an ethyl group, an n-propyl group, an isopropyl group, an n-butyl group, an s-butyl group, an isobutyl group, a t-butyl group, a cyclopentyl group, a silyl group, preferably SiPh₃, and a cyclohexyl group.

The optional substituent mentioned above may be further substituted by one or more of the optional substituents mentioned above.

The number of the optional substituents depends on the group which is substituted by said substituent(s). Preferred are 1, 2, 3 or 4 optional substituents, more preferred are 1, 2 or 3 optional substituents, most preferred are 1 or 2 optional substituents. In a further preferred embodiment, the groups mentioned above are unsubstituted.

The “carbon number of a to b” in the expression of “substituted or unsubstituted X group having a to b carbon atoms” is the carbon number of the unsubstituted X group and does not include the carbon atom(s) of an optional substituent.

The hydrogen atom referred to herein includes isotopes different from neutron numbers, i.e., light hydrogen (protium), heavy hydrogen (deuterium) and tritium.

The term “unsubstituted” referred to by “unsubstituted or substituted” means that a hydrogen atom is not substituted by one the groups mentioned above.

An index of 0 in the definition in any formula mentioned above and below means that a hydrogen atom is present at the position defined by said index.

The compounds of formula (I)

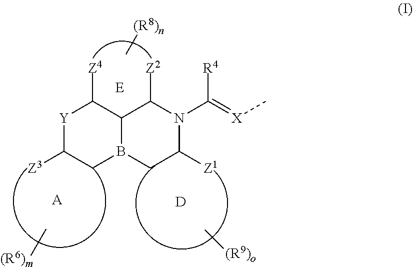

In the compounds of formula (I):

Ring A, ring E and ring D each independently represents an aromatic group having 6 to 30 ring carbon atoms or a heteroaromatic group having 3 to 30 ring atoms.

Preferably, ring A, ring E and ring D each independently represents an aromatic group having 6 to 18 ring carbon atoms or a heteroaromatic group having 3 to 16 ring atoms.

More preferably, ring A, ring E and ring D each independently represents a phenyl group, a naphthyl group, a phenanthrene group, a fluorene group, a triphenylene group, a spirobifluorene group, a fluoranthene group, an anthracene group, chrysene group, a dibenzofuran group, a carbazole group, or a dibenzothiophene group, a pyrrole group, an isoindole group, a benzofuran group, an isobenzofuran group, a benzothiophene group, an isoquinoline group, a quinoxaline group, a quinazoline group, a phenanthridine group, a phenanthroline group, a pyridine group, a pyrazine group, a pyrimidine group, a pyridazine group, an indole group, a quinoline group, an acridine group, a furan group, a thiophene group, a benzoxazole group, a benzothiazole group, a benzimidazole group, a 4-imidazo[1,2-a]benzimidazoyl group, a 5-benzimidazo[1,2-a]benzimidazoyl group, or a benzimidazolo[2,1-b][1,3]benzothiazolyl group.

Most preferably, ring A, ring E and ring D each independently represents a phenyl group, a naphthyl group, a phenanthrene group, a fluorene group, a triphenylene group, a dibenzofuran group, a carbazole group, a dibenzothiophene group, a pyridine group, or a pyrimidine group.

Further most preferably, ring A, ring E and ring D each represents a phenyl group, pyridine group or pyrimidine group.

Even further most preferably, ring A, ring E and ring D each represents a phenyl group or ring E is a phenyl group or a pyridine group and Rings A and D are phenyl groups.

The ring A may be substituted by m residues R⁶, or at the position Z³ by R^{X6A}, in the case that Z³ is ZR^{X6A}.

The ring E may be substituted by n residues R⁸, or at the position Z⁴ by R^{X8A}, in the case that Z⁴ is ZR^{X8A} or at the position Z² by R^{X8}, in the case that Z² is ZR^{X8}.

The ring D may be substituted by o residues R⁹, or at the position Z¹ by R^{X9}, in the case that Z¹ is ZR^{X9}.

R⁶, R⁸, R⁹, R^{X6A}, R^{X8A}, R^{X8} and R^{X9} each independently represents H, halogen, a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, a substituted or unsubstituted aryloxy group having 6 to 24 ring carbon atoms, a substituted or unsubstituted alkylthio group having 1 to 25 carbon atoms, a substituted or unsubstituted arylthio group having 6 to 24 ring carbon atoms, an alkyl and/or aryl substituted silyl group, an alkyl or aryl substituted carbonyl group, an alkyl or aryl substituted carboxyl group, alkyl or aryl substituted amide group, an alkyl, aryl or heteroaryl substituted amino group, a substituted phosphoryl group, CN, or a substituted or unsubstituted fluoroalkyl group having 1 to 25 carbon atoms;

wherein two adjacent groups R⁶, two adjacent groups R⁸ and/or two adjacent groups R⁹ and/or R^{X6A} and a group R⁶ adjacent to R^{X6A} and/or R^{X9} and a group R⁹ adjacent to R^{X9} and/or R^{X8} and a group R⁸ adjacent to R^{X8} and/or R^{X8A} and a group R⁸ adjacent to R^{X8A}, may form together an unsubstituted or substituted ring.

Preferably, R⁶, R⁸, R⁹, R^{X6A}, R^{X8A}, R^{X8} and R^{X9} each independently represents H, a substituted or unsubstituted alkyl group having 1 to 25 carbon atoms, a substituted or unsubstituted aryl group having 6 to 30 ring carbon atoms, a substituted or unsubstituted fluoroalkyl group having 1 to 25 carbon atoms, a substituted or unsubstituted heteroaryl group having 5 to 18 ring atoms, an alkyl and/or aryl substituted silyl group, an alkyl, aryl or heteroaryl substituted amino group, a substituted or unsubstituted alkoxy group having 1 to 25 carbon atoms, or a substituted or unsubstituted aryloxy group having 1 to 25 ring carbon atoms;

wherein two adjacent groups R⁶, two adjacent groups R⁸ and/or two adjacent groups R⁹ and/or R^{X6A} and a group R⁶ adjacent to R^{X6A} and/or R^{X9} and a group R⁹ adjacent to R^{X9} and/or R^{X8} and a group R⁸ adjacent to R^{X8} and/or R^{X8A} and a group R⁸ adjacent to R^{X8A}, may form together an unsubstituted or substituted ring.

More preferably, R⁶, R⁸, R⁹, R^{X6A}, R^{X8A}, R^{X8} and R^{X9} each independently represents H, a substituted or unsubstituted alkyl group having 1 to 8 carbon atoms, preferably methyl, ethyl, iso-propyl, n-propyl, n-butyl, iso-butyl, sec-butyl, n-pentyl, sec-pentyl, 3-pentyl, 2-methylbutyl, 3-methylbut-2-yl, 2-methylbut-2-yl or 2,2-dimethylpropyl; or a substituted or unsubstituted phenyl group, preferably unsubstituted phenyl; or a C₁-C₄-alkyl substituted phenyl, especially p-tert. butyl phenyl, mesityl, xylyl, O-methyl phenyl; or an unsubstituted or substituted biphenyl, preferably unsubstituted biphenyl; or phenyl substituted by halogen, especially 2,4-difluorophenyl; or a substituted or unsubstituted phenyloxy group, especially OPh, a substituted or unsubstituted diarylamino group, especially NPh₂ or N(C₅H₅Bu)₂; or a substituted or unsubstituted carbazolyl group linked via N;

wherein two adjacent groups R⁶, two adjacent groups R⁸ and/or two adjacent groups R⁹ and/or R^{X6A} and a group R⁶ adjacent to R^{X6A} and/or R^{X9} and a group R⁹ adjacent to R^{X9} and/or R^{X8} and a group R⁸ adjacent to R^{X8} and/or R^{X8A} and a group R⁸ adjacent to R^{X8A}, may form together an unsubstituted or substituted ring.

Most preferably, R⁶, R⁸, R⁹, R^{X6A}, R^{X8A}, R^{X8} and R^{X9} each independently represents H, or a substituted or unsubstituted phenyl group, preferably unsubstituted phenyl, C₁-C₄-alkyl substituted phenyl, especially p-tert. butyl phenyl, mesityl, xylyl, O-methyl phenyl, biphenyl; or phenyl substituted by halogen, especially 2,4-difluorophenyl.

Further most preferably, R⁶ and R^{X6A} are each independently H, tert-butyl, N-carbazolyl, N-tert-butyl-carbazolyl, xylyl or mesityl.

Further most preferably, R⁹ and R^{X9} are each independently H, tert-butyl or xylyl.

Further most preferably, R⁸, R^{X8} and R^{X8A} are each independently H, Me, F, CF₃ or OPh.

Suitable rings formed by two adjacent groups R⁶, two adjacent groups R⁸ and/or two adjacent groups R⁹ and/or R^{X6A} and a group R⁶ adjacent to R^{X6A} and/or R^{X9} and a group R⁹ adjacent to R^{X9} and/or R^{X8} and a group R⁸ adjacent to R^{X8} and/or R^{X8A} and a group R⁸ adjacent to R^{X8A} are for example the following rings (a) and (b):

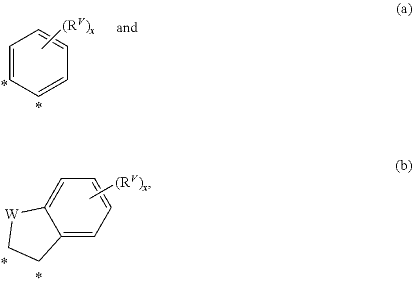

wherein

R^{V} represents H, a C₁-C₂₅ alkyl group, preferably a C₁-C₈ alkyl group, more preferably a C₁-C₄ alkyl group, or an unsubstituted or substituted C₆-C₃ aryl group, preferably unsubstituted or substituted C₆-C₁₀ aryl group, more preferably unsubstituted or substituted C aryl group, most preferably unsubstituted C aryl group;

x represents 0, 1, 2, 3 or 4, preferably 0, 1 or 2, more preferably 0; and

W represents CR′″₂, O, S or NR^{IV};

R′″ represents a C₁-C₂₅ alkyl group, preferably a C₁-C₈ alkyl group, more preferably a C₁-C₄ alkyl group;

R^{IV} represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group; and

each * represents a point of attachment to ring A, D or E.

n is 0 or 1.

m and o are each independently 0, 1, 2 or 3, preferably 0, 1 or 2.

X represents CR⁵ or N.

[symbol] the dotted line represents a single bond connected with Z¹ or connected with Z². Preferably, the dotted line represents a single bond connected with Z¹.

Z¹ represents C in the case that it is connected with the dotted line at X, and Z¹ represents CR^{X9} or N in the case that it is not connected with the dotted line at X. Preferably, Z¹ represents C, i.e. Z¹ is connected with the dotted line at X. Preferably, Z¹ represents CR^{X9} or N.

Z² represents C in the case that it is connected with the dotted line at X, and Z² represents CR^{X8} or N in the case that it is not connected with the dotted line at X. Preferably, Z² represents CR^{X8} or N.

In one preferred embodiment, X is CR⁵. In said embodiment, a group

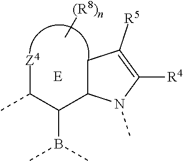

is formed with ring E (Z² represents C and is connected with the dotted line at X), respectively, a group

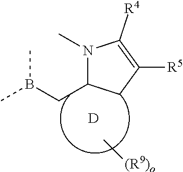

is formed with ring D (Z¹ represents C and is connected with the dotted line at X). The dotted lines in the figures above are boding sites to the rest of the compound of formula (I), whereby the formation of the group

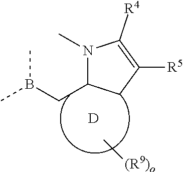

is preferred.

In a further preferred embodiment, X is N. In said embodiment, a group

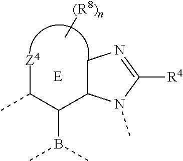

is formed with ring E (Z² represents C and is connected with the dotted line at X), respectively, a group

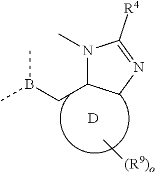

is formed with ring D (Z¹ represents C and is connected with the dotted line at X). The dotted lines in the figures above are boding sites to the rest of the compound of formula (I), whereby the formation of the group

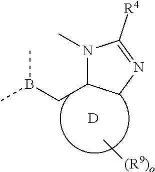

is preferred.

R⁴ and R⁵ each independently represents H, halogen, a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, a substituted or unsubstituted aryloxy group having 6 to 24 ring carbon atoms, a substituted or unsubstituted alkylthio group having 1 to 25 carbon atoms, a substituted or unsubstituted arylthio group having 6 to 24 ring carbon atoms, an alkyl and/or aryl substituted silyl group, an alkyl or aryl substituted carbonyl group, an alkyl, aryl or heteroaryl substituted amino group, an alkyl or aryl substituted amide group, an alkyl or aryl substituted carboxyl group, a substituted phosphoryl group, CN, or a substituted or unsubstituted fluoroalkyl group having 1 to 25 carbon atoms; or R⁴ and R⁵ may form together an unsubstituted or substituted aliphatic ring.

Preferably, R⁴ and R⁵ each independently represents H, a substituted or unsubstituted alkyl group having 1 to 25 carbon atoms, a substituted or unsubstituted aryl group having 6 to 30 ring carbon atoms, a substituted or unsubstituted fluoroalkyl group having 1 to 25 carbon atoms, a substituted or unsubstituted heteroaryl group having 5 to 18 ring atoms, an alkyl and/or aryl substituted silyl group, or an alkyl, aryl or heteroaryl substituted amino group; or

R⁴ and R⁵ together form a substituted or unsubstituted cyclohexyl ring.

More preferably, R⁴, R⁵ each independently represents a substituted or unsubstituted alkyl group having 1 to 8 carbon atoms, preferably methyl, ethyl, iso-propyl, n-propyl, n-butyl, iso-butyl, sec-butyl, n-pentyl, sec-pentyl, 3-pentyl, 2-methylbutyl, 3-methylbut-2-yl, 2-methylbut-2-yl or 2,2-dimethylpropyl; or a substituted or unsubstituted phenyl group, preferably unsubstituted phenyl, C₁-C₄-alkyl substituted phenyl, especially p-tert. butyl phenyl, mesityl, xylyl, O-methyl phenyl, xylyl, unsubstituted or substituted, preferably unsubstituted biphenyl, or a 2,4-difluorophenyl group;

or

R⁴ and R⁵ together form a substituted or unsubstituted cyclohexyl ring.

Y represents NR¹, O, S,

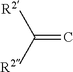

or CR²₂, preferably NR¹.

R¹ represents a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, or a substituted or unsubstituted fluoroalkyl group having 1 to 25 carbon atoms; or a group of formula

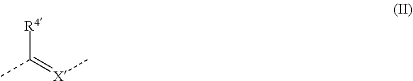

wherein X′ represents CR^{5′} or N; R^{4′}, R^{5′} and X′ are defined as R⁴, R⁵ and X; with the difference that R⁴ and R^{5′} may form together an unsubstituted or substituted ring;

[symbol] the dotted line at X′ represents a single bond connected with Z³ or connected with Z⁴;

Z³ represents C in the case that it is connected with the dotted line at X′, and Z³ represents CR^{X6A} or N in the case that it is not connected with the dotted line at X′;

Z⁴ represents C in the case that it is connected with the dotted line at X′, and Z⁴ represents CR^{X8A} or N in the case that it is not connected with the dotted line at X′;

[symbol] the other dotted line in the group of formula (II) represents a bonding site to the N atom of the group NR¹;

wherein in the case that R¹ represents a substituted cycloalkyl group having 3 to 25 ring carbon atoms, a substituted aryl group having 6 to 30 ring carbon atoms, preferably a substituted phenyl group, a substituted heteroaryl group having 3 to 18 ring atoms, or a substituted heterocyclic group having 3 to 18 ring atoms, one of the substituents of said groups may form together with R^{X6A} and/or R^{X8A} an unsubstituted or substituted ring.

Preferably, R¹ represents a substituted or unsubstituted alkyl group having 1 to 25 carbon atoms, a substituted or unsubstituted cycloalkyl group having 3 to 25 ring carbon atoms, a substituted or unsubstituted aryl group having 6 to 30 ring carbon atoms, a substituted or unsubstituted heteroaryl group having 3 to 18 ring atoms, or a substituted or unsubstituted heterocyclic group having 3 to 18 ring atoms; or

a group of formula

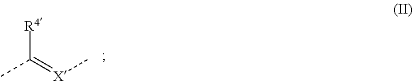

wherein in the case that R¹ represents a substituted cycloalkyl group having 3 to 25 ring carbon atoms, a substituted aryl group having 6 to 30 ring carbon atoms, preferably a substituted phenyl group, a substituted heteroaryl group having 3 to 18 ring atoms, or a substituted heterocyclic group having 3 to 18 ring atoms, one of the substituents of said groups may form together with R^{X6A} and/or R^{X8A} an unsubstituted or substituted ring.

More preferably, R¹ represents a substituted or unsubstituted aryl group having 6 to 30 ring carbon atoms, a substituted or unsubstituted heteroaryl group having 3 to 18 ring atoms; or

a group of formula

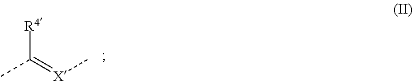

wherein in the case that R¹ represents a substituted aryl group having 6 to 30 ring carbon atoms, preferably a substituted phenyl group, or a substituted heteroaryl group having 3 to 18 ring atoms, one of the substituents of said groups may form together with R^{X6A} and/or R^{X8A} an unsubstituted or substituted ring;

Most preferably, R¹ represents a substituted or unsubstituted aryl group having 6 to 30 ring carbon atoms; or

a group of formula

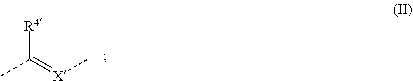

wherein in the case that R¹ represents a substituted aryl group having 6 to 30 ring carbon atoms, preferably a substituted phenyl group, one of the substituents of said groups may form together with R^{X6A} and/or R^{X8A} an unsubstituted or substituted ring.

Further most preferably, R¹ represents a substituted or unsubstituted phenyl group; or

a group of formula

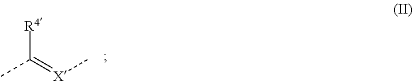

wherein in the case that R¹ represents a substituted phenyl group, one of the substituents of said groups may form together with R^{X6A} and/or R^{X8A} an unsubstituted or substituted ring.

In the case that R¹ may be connected to ring A or ring E, R¹ represents a substituted cycloalkyl group having 3 to 25 ring carbon atoms, a substituted aryl group having 6 to 30 ring carbon atoms, preferably a substituted phenyl group, a substituted heteroaryl group having 3 to 18 ring atoms, or a substituted heterocyclic group having 3 to 18 ring atoms, one of the substituents of said groups may form together with R^{X6A} and/or R^{X8A} an unsubstituted or substituted ring. Preferably, one of the substituents of said groups may form together with R^{X6A} and/or R^{X8A} a ring by one of the following bridging groups: single bond, —CR¹⁰²—, —NR¹¹—, or —C(R¹²)═C(R¹³)—, preferably a single bond. More preferably, the ring mentioned before may be formed in the case that R¹ is a substituted phenyl group.

In the group of formula (II), [symbol] the dotted line at X′ represents a single bond connected with Z³ or connected with Z⁴. Preferably, the dotted line represents a single bond connected with Z³

Z⁴ represents C in the case that it is connected with the dotted line at X′, and Z⁴ represents CR^{X8A} or N in the case that it is not connected with the dotted line at X′. Preferably, Z³ represents CR^{X6A} or N.

Z³ represents C in the case that it is connected with the dotted line at X′, and Z³ represents CR^{X6A} or N in the case that it is not connected with the dotted line at X′. Preferably, Z³ represents C, i.e. Z³ is connected with the dotted line at X′.

In one preferred embodiment, X′ is CR^{5′}. In said embodiment, a group

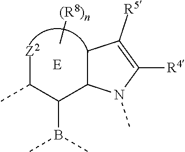

is formed with ring E (Z⁴ represents C and is connected with the dotted line at X′), respectively, a group

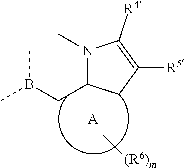

is formed with ring A (Z³ represents C and is connected with the dotted line at X′). The dotted lines in the figures above are boding sites to the rest of the compound of formula (I), whereby the formation of the group

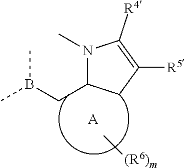

is preferred.

In a further preferred embodiment, X′ is N. In said embodiment, a group

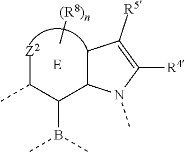

is formed with ring E (Z⁴ represents C and is connected with the dotted line at X′), respectively, a group

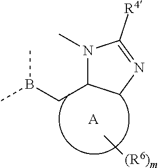

is formed with ring D (Z³ represents C and is connected with the dotted line at X′). The dotted lines in the figures above are boding sites to the rest of the compound of formula (I), whereby the formation of the group

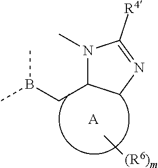

is preferred.

R², R^{2′} and R^{2″} each independently represents H, halogen, a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, a substituted or unsubstituted aryloxy group having 6 to 24 ring carbon atoms, a substituted or unsubstituted alkylthio group having 1 to 25 carbon atoms, a substituted or unsubstituted arylthio group having 6 to 24 ring carbon atoms, an alkyl and/or aryl substituted silyl group, an alkyl or aryl substituted carbonyl group, an alkyl, aryl or heteroaryl substituted amino group, an alkyl or aryl substituted amide group, an alkyl or aryl substituted carboxyl group, a substituted phosphoryl group, CN, or a substituted or unsubstituted fluoroalkyl group having 1 to 25 carbon atoms, or one of the residues R^{2′} or R^{2″} may be connected with ring A or ring E.

In the case that one of the residues R^{2′} or R^{2″} may be connected with ring A or ring E, the following groups are for example formed:

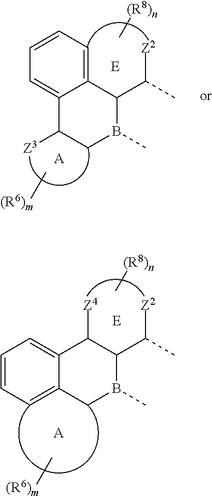

Preferably, R², R^{2′} and R^{2″} each independently represents a substituted or unsubstituted alkyl group having 1 to 25 carbon atoms, a substituted or unsubstituted cycloalkyl group having 3 to 25 ring carbon atoms, a substituted or unsubstituted aryl group having 6 to 30 ring carbon atoms, a substituted or unsubstituted heteroaryl group having 3 to 18 ring atoms, or a substituted or unsubstituted heterocyclic group having 3 to 18 ring atoms, or R^{2′} or R^{2″} may be connected with ring A or ring E as shown above.

More preferably, R², R^{2′} and R^{2″} each independently represents a substituted or unsubstituted alkyl group having 1 to 8 carbon atoms, a substituted or unsubstituted cycloalkyl group having 3 to 10 ring carbon atoms, a substituted or unsubstituted aryl group having 6 to 18 ring carbon atoms, a substituted or unsubstituted heteroaryl group having 5 to 14 ring atoms, or a substituted or unsubstituted heterocyclic group having 5 to 14 ring atoms, or R^{2′} or R^{2″} may be connected with ring A or ring E as shown above.

Most preferably, R², R^{2′} and R^{2″} each independently represents a substituted or unsubstituted alkyl group having 1 to 8 carbon atoms, a substituted or unsubstituted aryl group having 6 to 18 ring carbon atoms, or a substituted or unsubstituted heteroaryl group having 5 to 14 ring atoms or R^{2′} or R^{2″} may be connected with ring A or ring E as shown above.

Preferably, the ring A, the ring E and the ring D in the compounds of formula (I) each represents a phenyl group, a pyridine group or a pyrimidine group, more preferably ring A, ring E and ring D represents a phenyl group or ring E represents a phenyl group or a pyridyl group and ring A and ring D represents a phenyl group.

Preferred compounds according to the present invention are therefore represented by formula (III)

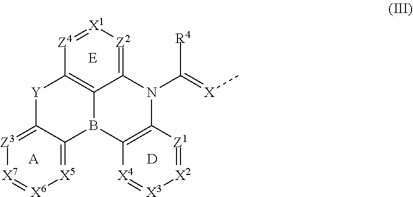

wherein

X¹ is CR^{X1} or N;

X² is CR^{X2} or N;

X³ is CR^{X3} or N;

X⁴ is CR^{X4} or N;

X⁵ is CR^{X5} or N;

X⁶ is CR^{X6} or N;

X⁷ is CR^{X7} or N;

R^{X1}, R^{X2}, R^{X3}, R^{X4}, R^{X5}, R^{X6} and R^{X7} are each independently defined as R⁶, R⁸ and R⁹ in formula (I); or

R^{X6A} and R^{X7}, R^{X6} and R^{X7}, R^{X5} and R^{X6}, R^{X4} and R^{X3}, R^{X2} and R^{X3}, R^{X2} and R^{X9}, R^{X8} and R^{X1}, and/or R^{X1} and R^{X8A}, may form together an unsubstituted or substituted ring. preferably, R^{X1}, R^{X2}, R^{X3}, R^{X4}, R^{X5}, R^{X6} and R^{X7} each independently represents H, halogen, a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, a substituted or unsubstituted aryloxy group having 6 to 24 ring carbon atoms, a substituted or unsubstituted alkylthio group having 1 to 25 carbon atoms, a substituted or unsubstituted arylthio group having 6 to 24 ring carbon atoms, an alkyl and/or aryl substituted silyl group, an alkyl or aryl substituted carbonyl group, an alkyl or aryl substituted carboxyl group, alkyl or aryl substituted amide group, an alkyl, aryl or heteroaryl substituted amino group, a substituted phosphoryl group, CN, or a substituted or unsubstituted fluoroalkyl group having 1 to 25 carbon atoms; or R^{X6A} and R^{X7}, R^{X6} and R^{X7}, R^{X5} and R^{X6}, R^{X4} and R^{X3}, R^{X2} and R^{X3}, R^{X2} and R^{X9}, R^{X8} and R^{X1}, and/or R^{X1} and R^{X8A}, may form together an unsubstituted or substituted ring.

More preferably, R^{X1}, R^{X2}, R^{X3}, R^{X4}, R^{X5}, R^{X6} and R^{X7} each independently represents H, a substituted or unsubstituted alkyl group having 1 to 25 carbon atoms, a substituted or unsubstituted aryl group having 6 to 30 ring carbon atoms, a substituted or unsubstituted fluoroalkyl group having 1 to 25 carbon atoms, a substituted or unsubstituted heteroaryl group having 5 to 18 ring atoms, an alkyl and/or aryl substituted silyl group, an alkyl, aryl or heteroaryl substituted amino group, a substituted or unsubstituted alkoxy group having 1 to 25 carbon atoms, a substituted or unsubstituted aryloxy group having 1 to 25 ring carbon atoms, or a substituted or unsubstituted fluoroalkyl group having 1 to 25 carbon atoms; or R^{X6A} and R^{X7}, R^{X6} and R^{X7}, R^{X5} and R^{X6}, R^{X4} and R^{X3}, R^{X2} and R^{X3}, R^{X2} and R^{X9}, R^{X8} and R^{X1}, and/or R^{X1} and R^{X8A}, may form together an unsubstituted or substituted ring.

Most preferably, R^{X1}, R^{X2}, R^{X3}, R^{X4}, R^{X5}, R^{X6} and R^{X7} each independently represents H, a substituted or unsubstituted alkyl group having 1 to 8 carbon atoms, preferably methyl, ethyl, iso-propyl, n-propyl, n-butyl, iso-butyl, sec-butyl, tert-butyl, n-pentyl, sec-pentyl, 3-pentyl, 2-methylbutyl, 3-methylbut-2-yl, 2-methylbut-2-yl or 2,2-dimethylpropyl; or a substituted or unsubstituted phenyl group, preferably unsubstituted phenyl; or a C₁-C₄-alkyl substituted phenyl, especially p-tert. butyl phenyl, mesityl, xylyl, O-methyl phenyl, substituted or unsubstituted biphenyl, preferably unsubstituted biphenyl; or phenyl substituted by halogen, especially 2,4-difluorophenyl; or a substituted or unsubstituted phenyloxy group, especially OPh; or a substituted or unsubstituted diarylamino group, especially NPh₂ or N(C₅H₅Bu)₂, a substituted or unsubstituted carbazolyl group linked via N, or a substituted or unsubstituted fluoroalkyl group having 1 to 4 carbon atoms, especially CF₃; or R^{X6A} and R^{X7}, R^{X6} and R^{X7}, R^{X5} and R^{X6}, R^{X4} and R^{X3}, R^{X2} and R^{X3}, R^{X2} and R^{X9}, R^{X8} and R^{X1}, and/or R^{X1} and R^{X8A}, may form together an unsubstituted or substituted ring.

Further most preferably, R^{X1}, R^{X2}, R^{X3}, R^{X4}, R^{X5}, R^{X6} and R^{X7} each independently represents H, a substituted or unsubstituted alkyl group having 1 to 4 carbon atoms, preferably methyl, ethyl, iso-propyl, n-propyl, n-butyl, iso-butyl, sec-butyl, tert-butyl, more preferably methyl or tert-butyl; or a substituted or unsubstituted phenyl group, preferably unsubstituted phenyl, C₁-C₄-alkyl substituted phenyl, especially p-tert. butyl phenyl, mesityl, xylyl, O-methyl phenyl, unsubstituted or substituted biphenyl, preferably unsubstituted biphenyl; or phenyl substituted by halogen, especially 2,4-difluorophenyl, or CF₃; or R^{X6A} and R^{X7}, R^{X6} and R^{X7}, R^{X5} and R^{X6}, R^{X4} and R^{X3}, R^{X2} and R^{X3}, R^{X2} and R^{X9}, R^{X8} and R^{X1}, and/or R^{X1} and R^{X8A}, may form together an unsubstituted or substituted ring.

Suitable rings formed by R^{X6A} and R^{X7}, R^{X6} and R^{X7}, R^{X5} and R^{X6}, R^{X4} and R^{X3}, R^{X2} and R^{X3}, R^{X2} and R^{X9}, R^{X8} and R^{X1}, and/or R^{X1} and R^{X8A} are for example the following rings (a) and (b):

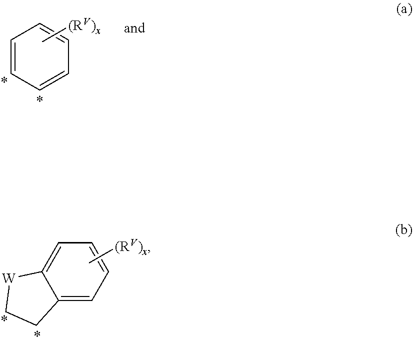

wherein

R^{V} represents H, a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group;

x represents 0, 1, 2, 3 or 4, preferably 0, 1 or 2, more preferably 0; and

W represents CR′″₂, O, S or NR^{IV};

R′″ represents a C₁-C₂₅ alkyl group, preferably a C₁-C₈ alkyl group, more preferably a C₁-C₄ alkyl group;

R^{IV} represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group; and

each * represents a point of attachment to ring A, D or E.

All other residues mentioned in formula (III) are defined as mentioned concerning formula (I) above.

Preferably, 0, 1, 2 or 3 of the residues R^{X1}, R^{X2}, R^{X3}, R^{X4}, R^{X5}, R^{X6} and R^{X7} each independently represent halogen, a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, a substituted or unsubstituted aryloxy group having 6 to 24 ring carbon atoms, a substituted or unsubstituted alkylthio group having 1 to 25 carbon atoms, a substituted or unsubstituted arylthio group having 6 to 24 ring carbon atoms, an alkyl and/or aryl substituted silyl group, an alkyl or aryl substituted carbonyl group, an alkyl or aryl substituted carboxyl group, alkyl or aryl substituted amide group, an alkyl, aryl or heteroaryl substituted amino group, a substituted phosphoryl group, CN, or a substituted or unsubstituted fluoroalkyl group having 1 to 25 carbon atoms; or R^{X6A} and R^{X7}, R^{X6} and R^{X7}, R^{X5} and R^{X6}, R^{X4} and R^{X3}, R^{X2} and R^{X3}, R^{X2} and R^{X9}, R^{X8} and R^{X1}, and/or R^{X1} and R^{X8A}, may form together an unsubstituted or substituted ring; preferably a substituted or unsubstituted alkyl group having 1 to 25 carbon atoms; a substituted or unsubstituted aryl group having 6 to 30 ring carbon atoms, a substituted or unsubstituted fluoroalkyl group having 1 to 25 carbon atoms, a substituted or unsubstituted heteroaryl group having 5 to 18 ring atoms, an alkyl and/or aryl substituted silyl group, an alkyl, aryl or heteroaryl substituted amino group, a substituted or unsubstituted alkoxy group having 1 to 25 carbon atoms, or a substituted or unsubstituted aryloxy group having 1 to 25 ring carbon atoms, or a substituted or unsubstituted fluoroalkyl group having 1 to 25 carbon atoms; or R^{X6A} and R^{X7}, R^{X6} and R^{X7}, R^{X5} and R^{X6}, R^{X4} and R^{X3}, R^{X2} and R^{X3}, R^{X2} and R^{X9}, R^{X8} and R^{X1}, and/or R^{X1} and R^{X8A}, may form together an unsubstituted or substituted ring; more preferably a substituted or unsubstituted alkyl group having 1 to 8 carbon atoms, preferably methyl, ethyl, iso-propyl, n-propyl, n-butyl, iso-butyl, sec-butyl, tert-butyl, n-pentyl, sec-pentyl, 3-pentyl, 2-methylbutyl, 3-methylbut-2-yl, 2-methylbut-2-yl or 2,2-dimethylpropyl; or a substituted or unsubstituted phenyl group, preferably unsubstituted phenyl, C₁-C₄-alkyl substituted phenyl, especially p-tert. butyl phenyl, mesityl, xylyl, O-methyl phenyl, substituted or unsubstituted biphenyl, preferably unsubstituted biphenyl; or phenyl substituted by halogen, especially 2,4-difluorophenyl; a substituted or unsubstituted phenyloxy group, especially OPh, a substituted or unsubstituted diarylamino group, especially NPh₂ or N(C₅H₅Bu)₂, a substituted or unsubstituted carbazolyl group linked via N, or a substituted or unsubstituted fluoroalkyl group having 1 to 4 carbon atoms, especially CF₃; or R^{X6A} and R^{X7}, R^{X6} and R^{X7}, R^{X5} and R^{X6}, R^{X4} and R^{X3}, R^{X2} and R^{X3}, R^{X2} and R^{X9}, R^{X8} and R^{X1}, and/or R^{X1} and R^{X8A}, may form together an unsubstituted or substituted ring; most preferably a substituted or unsubstituted alkyl group having 1 to 4 carbon atoms, preferably methyl, ethyl, iso-propyl, n-propyl, n-butyl, iso-butyl, sec-butyl, tert-butyl, more preferably methyl or tert-butyl; a substituted or unsubstituted phenyl group, preferably unsubstituted phenyl, C₁-C₄-alkyl substituted phenyl, especially p-tert. butyl phenyl, mesityl, xylyl, O-methyl phenyl, substituted or unsubstituted biphenyl, preferably unsubstituted biphenyl; phenyl substituted by halogen, especially 2,4-difluorophenyl, or CF₃; or R^{X6A} and R^{X7}, R^{X6} and R^{X7}, R^{X5} and R^{X6}, R^{X4} and R^{X3}, R^{X2} and R^{X3}, R^{X2} and R^{X9}, R^{X8} and R^{X1}, and/or R^{X1} and R^{X8A}, may form together an unsubstituted or substituted ring; and the other of the residues R^{X1}, R^{X2}, R^{X3}, R^{X4}, R^{X5}, R^{X6} and R^{X7} represent hydrogen.

Further most preferably, R^{X6A}, R^{X8} and R^{X9} in formula (III) are H, R^{X8A} is H or CF₃, and the residues R^{X1}, R^{X2}, R^{X3}, R^{X4}, R^{X5}, R^{X6} and R^{X7} are as defined above.

Further preferably, in formula (III)

X′ is CR^{X1}; X² is CR^{X2}; X³ is CR^{X3}; X⁴ is CR^{X4}; X⁵ is CR^{X5}; X⁶ is CR^{X6}; X⁷ is CR^{X7}.

More preferred compounds of the present invention are therefore represented by formula (IV)

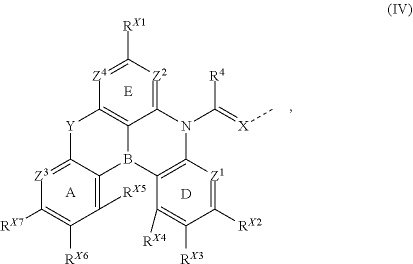

wherein the groups and residues are defined above, in the definition of formula (III).

[symbol] the dotted line represents a single bond connected with Z¹ or connected with Z².

In the case that the dotted line represents a single bond connected with Z¹, a compound of formula (V) is formed:

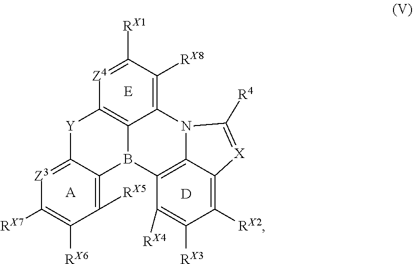

wherein the groups and residues are defined above, in the definition of formula (III).

In the case that the dotted line represents a single bond connected with Z², a compound of formula (VI) is formed:

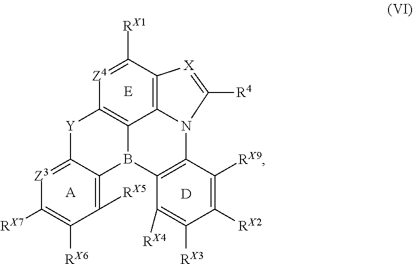

wherein the groups and residues in formulae (V) and (VI) are defined above, in the definition of formula (III).

Preferably, Y in formulae (Ill), (IV), (V) and (VI) represents NR¹, wherein R¹ is defined as mentioned above.

More preferred compounds according to the present invention are therefore compounds of formulae (Va) and (VIa)

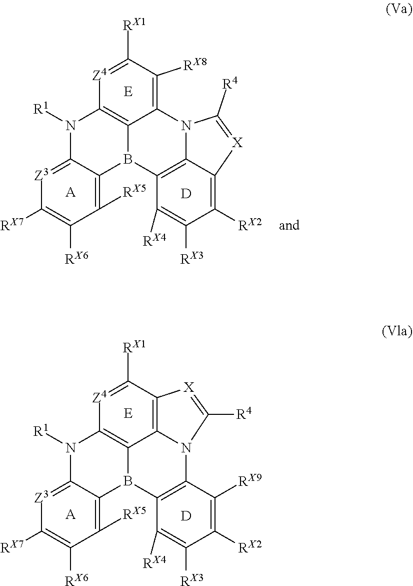

wherein the groups and residues in formulae (Va) and (VIa) are defined above in the definition of formula (III), and R¹ is defined above.

In a most preferred embodiment, R¹ represents a substituted or unsubstituted phenyl group; or

a group of formula

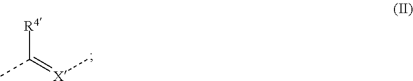

wherein in the case that R¹ represents a substituted phenyl group, one of the substituents of said groups may form together with R^{X6A} and/or R^{X8A} an unsubstituted or substituted ring, wherein the residue R^{4′} and the group X′ are defined above.

Most preferred compounds are therefore represented by the following formulae (VII), (VIII), (IX), (X) and (XI)

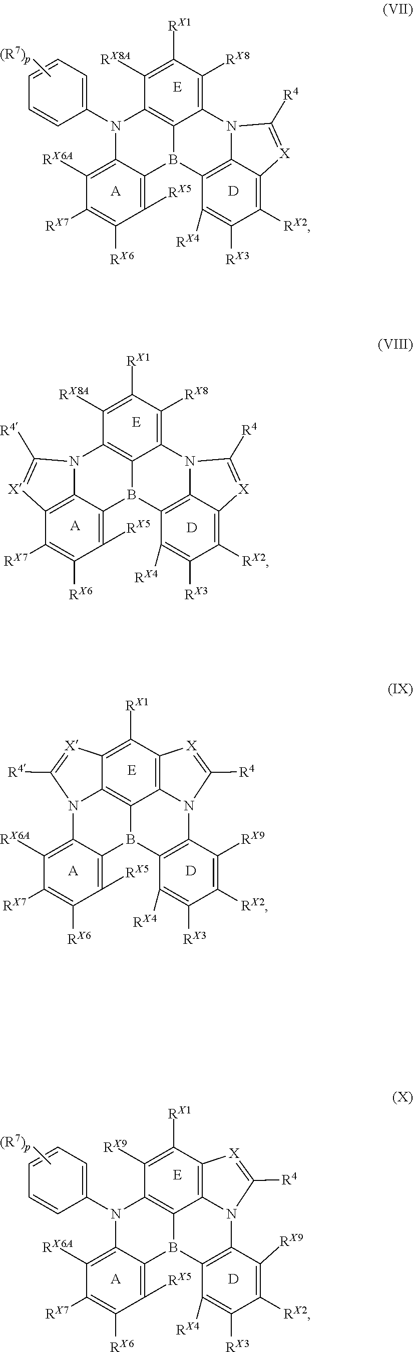

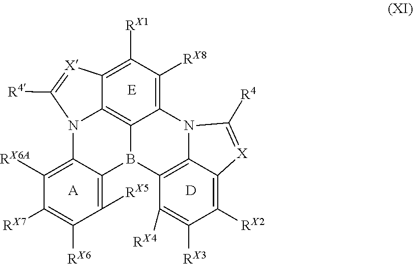

wherein

R⁷ represents H, halogen, a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, a substituted or unsubstituted aryloxy group having 6 to 24 ring carbon atoms, a substituted or unsubstituted alkylthio group having 1 to 25 carbon atoms, a substituted or unsubstituted arylthio group having 6 to 24 ring carbon atoms, an alkyl and/or aryl substituted silyl group, an alkyl or aryl substituted carbonyl group, an alkyl or aryl substituted carboxyl group, alkyl or aryl substituted amide group, an alkyl, aryl or heteroaryl substituted amino group, a substituted phosphoryl group, CN, or a substituted or unsubstituted fluoroalkyl group having 1 to 25 carbon atoms;

wherein two adjacent groups R⁷ may form together an unsubstituted or substituted ring or

R⁷ and R^{X6A} and/or R⁷ and R^{X8A} may form together an unsubstituted or substituted ring, p represents, 0, 1, 2, 3, 4 or 5, preferably 0, 1, 2 or 3, more preferably 0, 1 or 2. and

the other groups and residues in the formulae (VII), (VIII), (IX), (X) and (XI) are defined above.

Preferred compounds of formulae (VII) and (X), wherein R⁷ and R^{X6A} and/or R⁷ and R^{X8A} form together an unsubstituted or substituted ring are for example compounds of the following formulae:

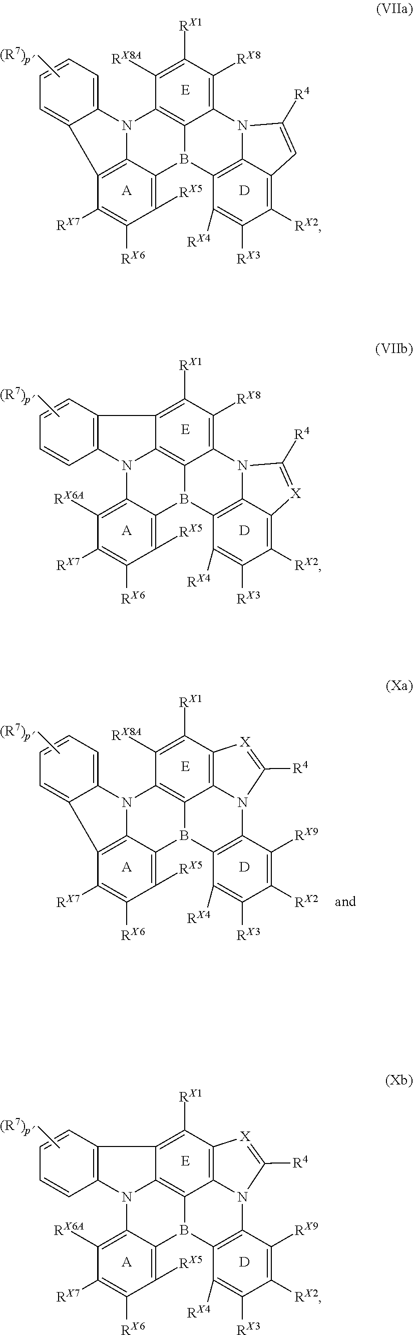

wherein p′ is 0, 1, 2, 3 or 4, preferably 0, 1 or 2, more preferably 0 or 1, and the further groups and residues in the formulae (VIIa), (VIIb), (Xa) and (Xb) are defined above.

X in the compounds according to formulae (VII), (VIII), (IX), (X) and (XI) represents CR⁵ or N, wherein R⁵ is defined above.

X′ in the compounds according to formulae (VII), (VIII), (IX), (X) and (XI) represents represents CR^{5′} or N, wherein R^{5′} is defined above.

Further most preferred compounds are therefore represented by the following formulae (XII), (XIII), (XIV), (XV), (XVI), (XVII), (XVIII), (XIX), (XX), (XXI), (XXII), (XXIII), (XXIV) and (XXV)

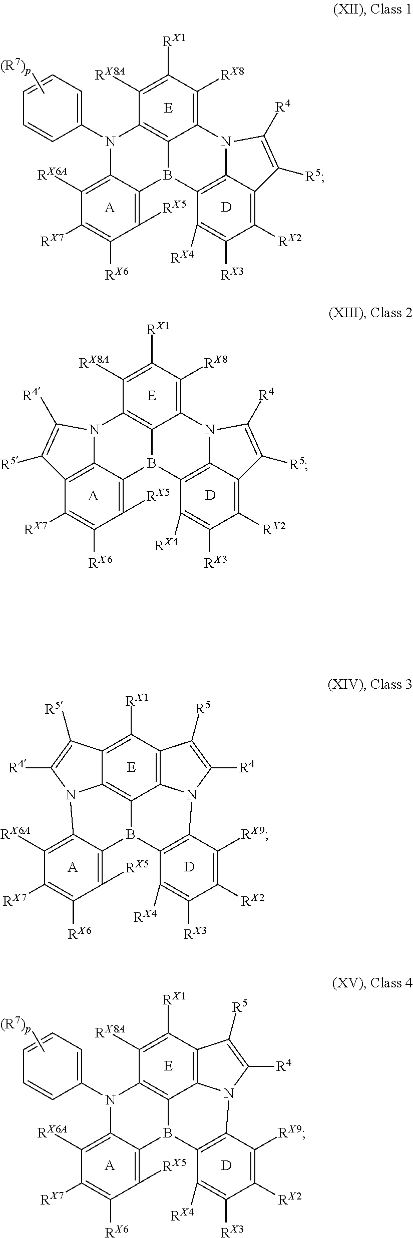

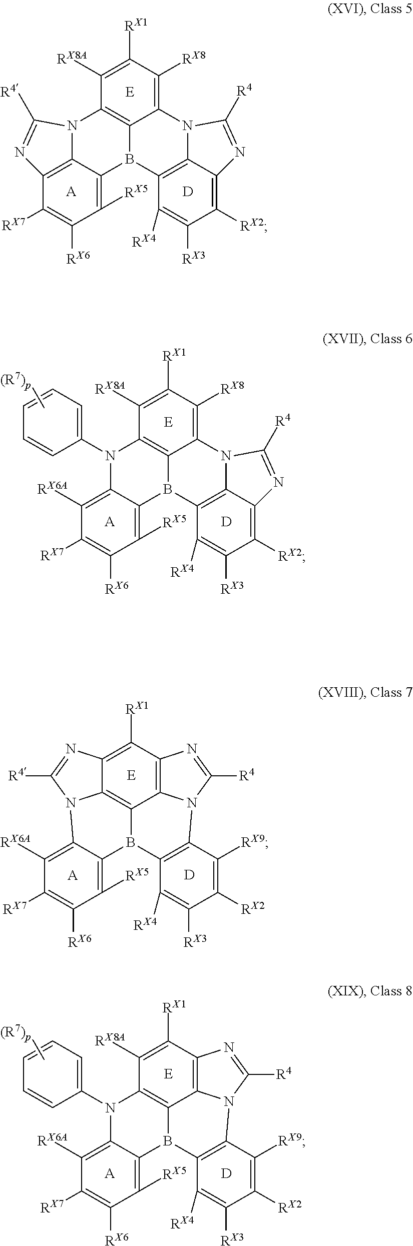

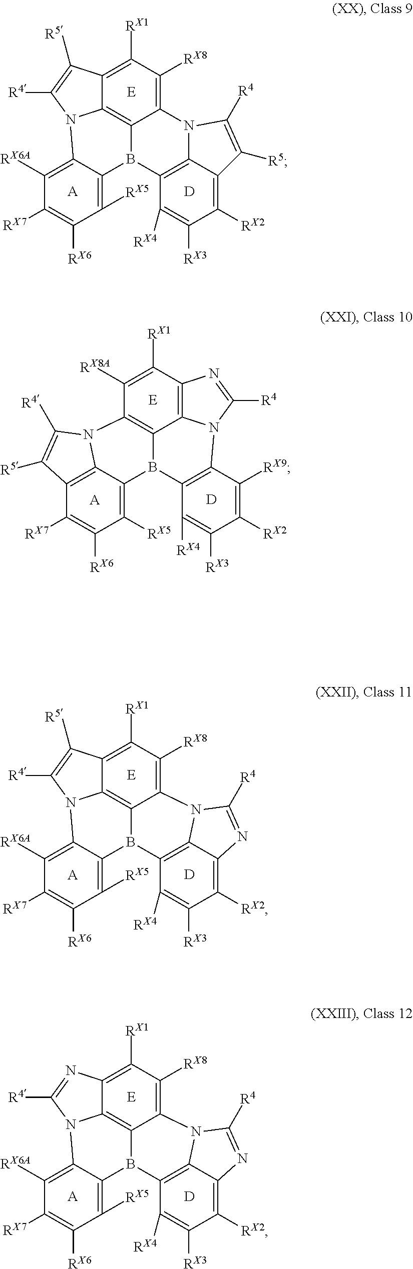

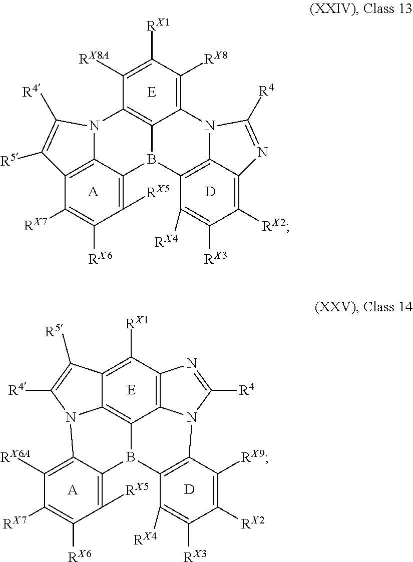

wherein the groups and residues mentioned in (XII), (XIII), (XIV), (XV), (XVI), (XVII), (XVIII), (XIX), (XX), (XXI), (XXII), (XXIII), (XXIV) and (XXV) are defined above.

Preferred compounds are the compounds of formulae (XII) (=Class 1); (XVII) (=Class 6); (XVI) (=Class 5); (XIII) (=Class 2); (XXIII) (=Class 12); (XVIII) (=Class 7); and (XIV) (=Class 13). More preferred compounds are the compounds of formulae formulae (XII) (=Class 1); (XVII) (=Class 6); (XVI) (=Class 5); (XIII) (=Class 2); and (XXIII) (=Class 12). Most preferred compounds are the compounds of formulae (XII) (=Class 1); and (XVII) (=Class 6).

In the case that X is CR⁵ and X′ is CR^{5′}, the compounds of formulae (XII) (=Class 1); and (XIII) (=Class 2) are preferred, and the compounds of formula (XII) (=Class 1) are more preferred.

In the case that X and X′ are N, the compounds of formulae (XVII) (=Class 6); (XVI) (=Class 5); and (XXIII) (=Class 12) are preferred, and the compounds of formula (XVII) (=Class 6) are more preferred.

Preferably, in the compounds of the present invention, R⁴, R⁵, R^{4′} and R^{5′} each independently represents H, a substituted or unsubstituted alkyl group having 1 to 25 carbon atoms, a substituted or unsubstituted aryl group having 6 to 30 ring carbon atoms, a substituted or unsubstituted fluoroalkyl group having 1 to 25 carbon atoms, a substituted or unsubstituted heteroaryl group having 5 to 18 ring atoms, an alkyl and/or aryl substituted silyl group, or an alkyl, aryl or heteroaryl substituted amino group;

or

R⁴ and R⁵ together form a substituted or unsubstituted cyclohexene ring; and/or

R^{4′} and R^{5′} together form a substituted or unsubstituted phenyl ring or a substituted or unsubstituted cyclohexene ring;

preferably, R⁴, R⁵, R^{4′} and R^{5′} each independently represents a substituted or unsubstituted alkyl group having 1 to 8 carbon atoms, preferably methyl, ethyl, iso-propyl, n-propyl, n-butyl, iso-butyl, sec-butyl, tert-butyl, n-pentyl, sec-pentyl, 3-pentyl, 2-methylbutyl, 3-methylbut-2-yl, 2-methylbut-2-yl or 2,2-dimethylpropyl; or a substituted or unsubstituted phenyl group, preferably unsubstituted phenyl, C₁-C₄-alkyl substituted phenyl, especially p-tert. butyl phenyl, mesityl, xylyl, O-methyl phenyl, substituted or unsubstituted biphenyl, preferably unsubstituted biphenyl, or a 2,4-difluorophenyl group;

or

R⁴ and R⁵ together form a substituted or unsubstituted cyclohexene ring;

and/or

R^{4′} and R^{5′} together form a substituted or unsubstituted phenyl ring or a substituted or unsubstituted cyclohexene ring.

Preferably, in the compounds of the present invention, R^{X1} and R^{X8}, R⁶, R⁷, R⁸, R⁹, R^{X2}, R^{X3}, R^{X4}, R^{X5}, R^{X6}, R^{X7}, R^{X6A}, R^{X8A} and R^{X9} each independently represents H, a substituted or unsubstituted alkyl group having 1 to 25 carbon atoms; a substituted or unsubstituted aryl group having 6 to 30 ring carbon atoms, a substituted or unsubstituted fluoroalkyl group having 1 to 25 carbon atoms, a substituted or unsubstituted heteroaryl group having 5 to 18 ring atoms, an alkyl and/or aryl substituted silyl group, an alkyl, aryl or heteroaryl substituted amino group, a substituted or unsubstituted alkoxy group having 1 to 25 carbon atoms, or a substituted or unsubstituted aryloxy group having 1 to 25 ring carbon atoms; or R^{X6A} and R^{X7}, R^{X6} and R^{X7}, R^{X5} and R^{X6}, R^{X4} and R^{X3}, R^{X2} and R^{X3}, R^{X2} and R^{X9}, R^{X8} and R^{X1}, and/or R^{X1} and R^{X8A}, may form together an unsubstituted or substituted ring; or R⁷ and R^{X6A} and/or R⁷ and R^{X8A} may form together a ring by one of the following bridging groups: single bond, —CR¹⁰²—, —NR¹¹—, or —C(R¹²)═C(R¹³)—;

wherein

R¹⁰ represents H or a substituted or unsubstituted alkyl group having 1 to 8 carbon atoms;

R¹¹ represents a substituted or unsubstituted aryl group having 6 to 30 ring carbon atoms or a substituted or unsubstituted heteroaryl group having 5 to 18 ring atoms; and

R¹² and R¹³ each independently represents H, a substituted or unsubstituted alkyl group having 1 to 8 carbon atoms, a substituted or unsubstituted aryl group having 6 to 18 ring carbon atoms or a substituted or unsubstituted heteroaryl group having 5 to 14 ring atoms; or

R¹² and R¹³ together form a substituted or unsubstituted carbocyclic or heterocyclic ring comprising 5 or 6 ring atoms;

preferably, R⁶, R⁷, R⁸, R⁹, R^{X2}, R^{X3}, R^{X4}, R^{X5}, R^{X6}, R^{X7}, R^{X9}, R^{X6A} and R^{X8A} each independently represents H, a substituted or unsubstituted alkyl group having 1 to 8 carbon atoms, preferably methyl, ethyl, iso-propyl, n-propyl, n-butyl, iso-butyl, sec-butyl, tert-butyl, n-pentyl, sec-pentyl, 3-pentyl, 2-methylbutyl, 3-methylbut-2-yl, 2-methylbut-2-yl or 2,2-dimethylpropyl; or a substituted or unsubstituted phenyl group, preferably unsubstituted phenyl, C₁-C₄-alkyl substituted phenyl, especially p-tert. butyl phenyl, mesityl, xylyl, O-methyl phenyl, substituted or unsubstituted biphenyl, preferably unsubstituted biphenyl; or phenyl substituted by halogen, especially 2,4-difluorophenyl;

R^{X1} and R^{X8} each independently represents H, a substituted or unsubstituted alkyl group having 1 to 8 carbon atoms, preferably methyl, ethyl, iso-propyl, n-propyl, n-butyl, iso-butyl, sec-butyl, tert-butyl, n-pentyl, sec-pentyl, 3-pentyl, 2-methylbutyl, 3-methylbut-2-yl, 2-methylbut-2-yl or 2,2-dimethylpropyl; or a substituted or unsubstituted phenyl group, preferably unsubstituted phenyl, C₁-C₄-alkyl substituted phenyl, especially p-tert. butyl phenyl, mesityl, xylyl, O-methyl phenyl, substituted or unsubstituted biphenyl, preferably unsubstituted biphenyl; or phenyl substituted by halogen, especially 2,4-difluorophenyl; a substituted or unsubstituted phenyloxy group, especially OPh, a substituted or unsubstituted diarylamino group, especially NPh₂ or N(C₅H₅Bu)₂, or a substituted or unsubstituted carbazolyl group linked via N;

or R^{X6A} and R^{X7}, R^{X6} and R^{X7}, R^{X5} and R^{X6}, R^{X4} and R^{X3}, R^{X2} and R^{X3}, R^{X2} and R^{X9}, R^{X8} and R^{X1}, and/or R^{X1} and R^{X8A}, may form together an unsubstituted or substituted ring;

or

R⁷ and R^{X6A} and/or R⁷ and R^{X8A} may form together a ring by one of the following bridging groups: single bond, —CR¹⁰²—, —NR¹¹—, or —C(R¹²)═C(R¹³)—, preferably a single bond;

wherein

R¹⁰ represents H, methyl, ethyl, iso-propyl, n-propyl, n-butyl, iso-butyl, sec-butyl, tert-butyl, n-pentyl, sec-pentyl, 3-pentyl, 2-methylbutyl, 3-methylbut-2-yl, 2-methylbut-2-yl or 2,2-dimethylpropyl;

R¹¹ represents a substituted or unsubstituted phenyl group or a substituted or unsubstituted heteroaryl group having 5 to 10 ring atoms; and

R¹² and R¹³ each independently represents H, methyl, ethyl, iso-propyl, n-propyl, n-butyl, iso-butyl, sec-butyl, tert-butyl, n-pentyl, sec-pentyl, 3-pentyl, 2-methylbutyl, 3-methylbut-2-yl, 2-methylbut-2-yl or 2,2-dimethylpropyl, a substituted or unsubstituted phenyl group or a substituted or unsubstituted heteroaryl group having 5 to 10 ring atoms; or

R¹² and R¹³ together form a substituted or unsubstituted aromatic ring comprising 6 ring atoms.

The compounds of formula

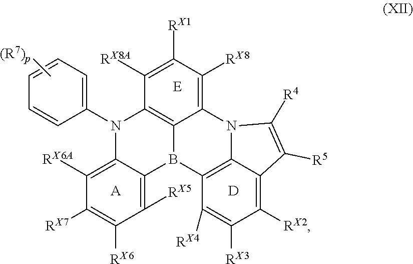

### Class 1

In the compounds of class 1 (formula (XII)), the groups, residues and indices R⁴, R⁵, R⁷, R^{X1}, R^{X2}, R^{X3}, R^{X4}, R^{X5}, R^{X6}, R^{X6A}_R^{X7}, R^{X8}, R^{X9} and p are defined above.

In the case that R^{X6A} and R^{X7}, R^{X6} and R^{X7}, R^{X5} and R^{X6}, R^{X4} and R^{X3}, R^{X2} and R^{X3}, R^{X8} and R^{X1}, and/or R^{X1} and R^{X8A}, or two adjacent residues R⁷ in the compounds of class 1 may form together an unsubstituted or substituted ring, the following rings (a) and (b) are formed:

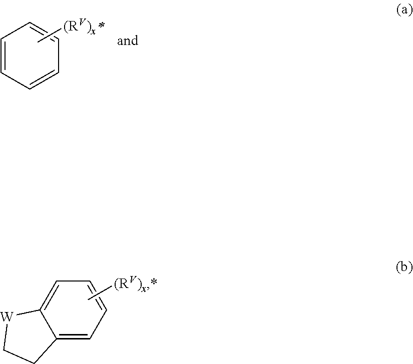

wherein

R^{V} represents H, a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group;

x represents 0, 1, 2, 3 or 4, preferably 0, 1 or 2, more preferably 0; and

W represents CR′″₂, O, S or NR^{IV};

R′″ represents a C₁-C₂₅ alkyl group, preferably a C₁-C₈ alkyl group, more preferably a C₁-C₄ alkyl group;

R^{IV} represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group; and

each * represents a point of attachment to ring A, D or E, or to the phenyl ring bearing the R⁷-substituent(s).

Most preferably, R⁴ and R⁵ in the compounds of class 1 each independently represents methyl, ethyl, iso-propyl, sec-propyl, n-butyl, —C(Me)₂C₂H₅, unsubstituted phenyl, p-tert-butyl-phenyl, mesityl, xylyl, O-methyl-phenyl or unsubstituted or substituted biphenyl, preferably unsubstituted biphenyl; or

R⁴ and R⁵ may form together an unsubstituted or substituted cyclohexene ring, preferably an unsubstituted cyclohexene ring.

An example for compounds of class 1, wherein R⁴ and R⁵ may form together an unsubstituted cyclohexene ring is the following compound:

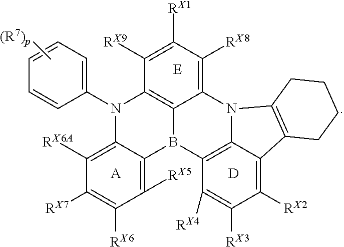

Most preferably, R^{X4} and R^{X5} in the compounds of class 1 are H.

Most preferably, R^{X6}, R^{X6A}, R^{X7} and R⁷ in the compounds of class 1 each independently represents H, methyl, ethyl, iso-propyl, sec-propyl, n-butyl, tert-butyl, —C(Me)₂C₂H₅, unsubstituted phenyl, p-tert-butyl-phenyl, mesityl, xylyl, O-methyl-phenyl, unsubstituted or substituted biphenyl, preferably unsubstituted biphenyl or 2,4-difluoro(2,4-difluorophenyl); or R^{X6A} and one of the residues R⁷ in ortho position to the nitrogen atom, may form together a ring, wherein the ring is formed via a single bond, via a C₁-C₃ alkyl group which is optionally substituted by a C₁-C₂₅ alkyl group, preferably by a C₁-C₈ alkyl group, more preferably by a C₁-C₄ alkyl group, via an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group, via an unsubstituted or substituted C₂ alkenyl group, via a group NR′, via O, via a group POOR′ or via a unsubstituted or substituted P—C₆-C₃₀ aryl group, preferably unsubstituted or substituted P—C₆-C₁₀ aryl group, more preferably unsubstituted or substituted P—C₆ aryl group, most preferably unsubstituted P—C₆ aryl group; preferably via a single bond, wherein

R′ represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group.

Examples for compounds of class 1, wherein R^{X6A} and one of the residues R⁷ in ortho position to the nitrogen atom, may form together a ring are the following compounds:

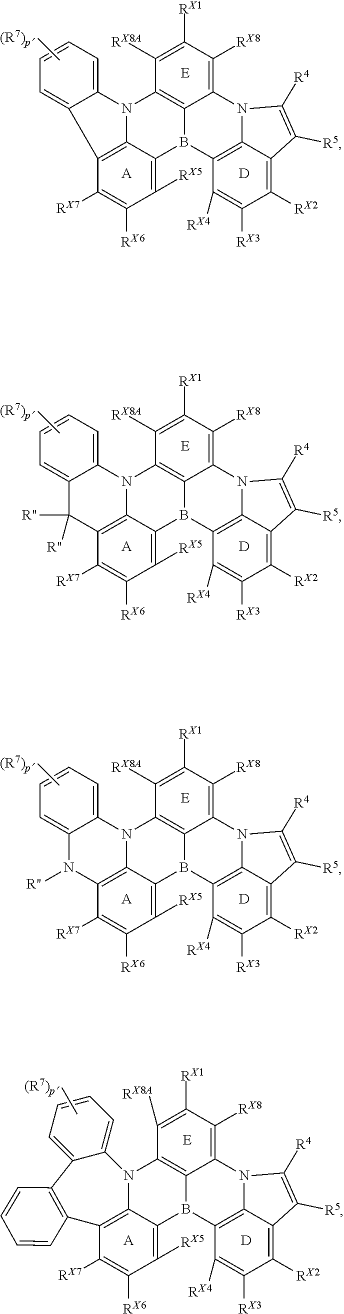

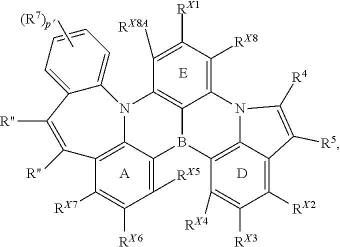

wherein

p′ is 0, 1, 2, 3 or 4, preferably 0, 1 or 2, more preferably 0 or 1;

R″ each independently represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C aryl group, most preferably unsubstituted C aryl group;

and the other groups, residues and indices are as defined in formula (XII).

Most preferably, R^{X1}, R^{X8} and R^{X8A} in the compounds of class 1 each independently represents H, methyl, ethyl, iso-propyl, sec-propyl, n-butyl, tert-butyl, —C(Me)₂C₂H₅, unsubstituted phenyl, p-tert-butyl-phenyl, mesityl, xylyl, O-methyl-phenyl, unsubstituted or substituted biphenyl, preferably unsubstituted biphenyl or 2,4-difluoro(2,4-difluorophenyl), OPh; NPh₂, N-carbazoyl, N(C₆H₅tBu)₂; or

R^{X8A} and one of the residues R⁷ in ortho position to the nitrogen atom, may form together a ring, wherein the ring is formed via a single bond, via a C₁-C₃ alkyl group which is optionally substituted by a C₁-C₂₅ alkyl group, preferably by a C₁-C₈ alkyl group, more preferably by a C₁-C₄ alkyl group, via an unsubstituted or substituted C₆-C₀ aryl group, preferably unsubstituted or substituted C₆-C₁₀ aryl group, more preferably unsubstituted or substituted C₆ aryl group, most preferably unsubstituted C₆ aryl group, via an unsubstituted or substituted C₂ alkenyl group, via a group NR′, via O, via a group POOR′ or via a unsubstituted or substituted P—C₆-C₃₀ aryl group, preferably unsubstituted or substituted P—C₆-C₁₀ aryl group, more preferably unsubstituted or substituted P—C₆ aryl group, most preferably unsubstituted P—C₆ aryl group; preferably via a single bond,

wherein

R′ represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group.

Examples for compounds of class 1, wherein R^{X8A} and one of the residues R⁷ in ortho position to the nitrogen atom, may form together a ring are the following compounds:

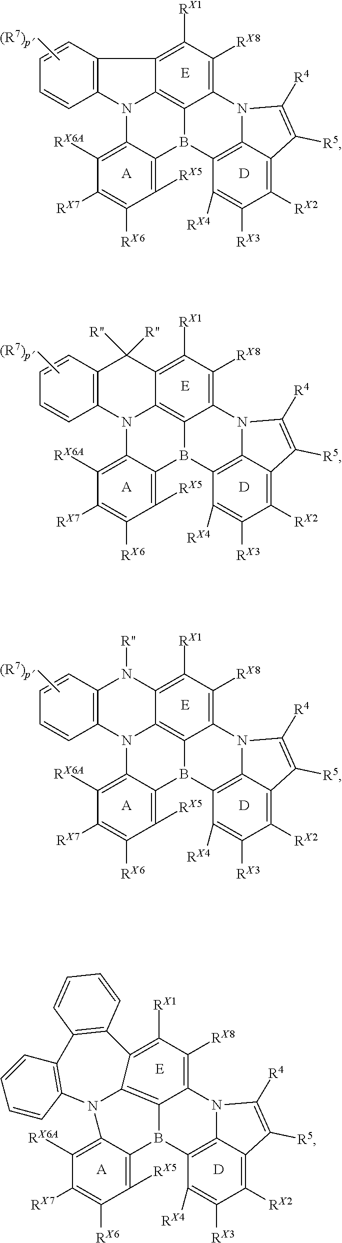

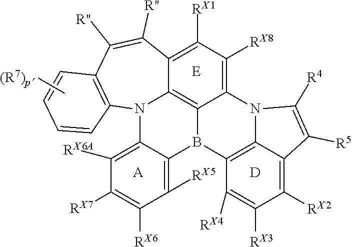

wherein

p′ is 0, 1, 2, 3 or 4, preferably 0, 1 or 2, more preferably 0 or 1;

R″ each independently represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C aryl group, most preferably unsubstituted C aryl group; and the other groups, residues and indices are as defined in formula (XII).

Most preferably, R^{X2}, R^{X3} and R^{X4} in the compounds of class 1 each independently represents H, methyl, ethyl, iso-propyl, sec-propyl, n-butyl, tert-butyl, —C(Me)₂C₂H₅, unsubstituted phenyl, p-tert-butyl-phenyl, mesityl, xylyl, O-methyl-phenyl, unsubstituted or substituted biphenyl, preferably unsubstituted biphenyl or 2,4-difluoro(2,4-difluorophenyl).

Examples for compounds of class 1 are compounds of the following formulae (A) and (B), wherein the residues R⁴, R⁵, R^{X1}, R^{X6} and R⁷ are defined above.

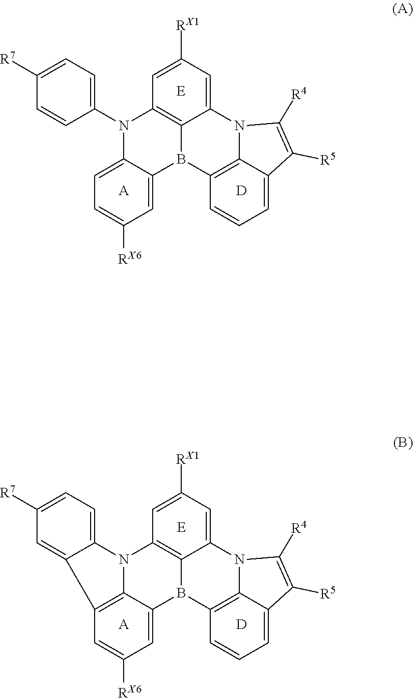

Preferred compounds (A) and (B) are mentioned in the following table:

| R^{X6} | R⁷ | R^{X1} | R⁴ | R⁵ |
|---|---|---|---|---|
| H | H | H | Ph | Ph |
| tBu | tBu | H | Ph | Ph |
| tBu | tBu | H | Me | Me |
| H | H | H | Me | Me |
| H | H | H | Me | H |
| H | H | H | H | Me |
| tBu | tBu | H | H | Me |
| tBu | tBu | H | H | tBu |
| tBu | tBu | H | Me | H |
| tBu | tBu | H | tBu | H |
| H | H | H | H | Ph |
| tBu | tBu | H | H | Ph |
| H | H | H | Ph | H |
| tBu | tBu | H | Ph | H |
| D | D | D | Ph | Ph |
| tBu | tBu | D | Ph | Ph |
| tBu | tBu | D | Me | Me |
| D | D | D | Me | Me |
| D | D | D | Me | H |
| D | D | D | H | Me |
| tBu | tBu | D | H | Me |
| tBu | tBu | D | H | tBu |
| tBu | tBu | D | Me | H |
| tBu | tBu | D | tBu | H |
| D | D | D | H | Ph |
| tBu | tBu | D | H | Ph |
| D | D | D | Ph | H |
| tBu | tBu | D | Ph | H |

Examples for compounds of class 1 are compounds of the following formulae (C) and (D), wherein the residues R⁴, R⁵, R^{X1}, R^{X3}, R^{X6} and R⁷ are defined above.

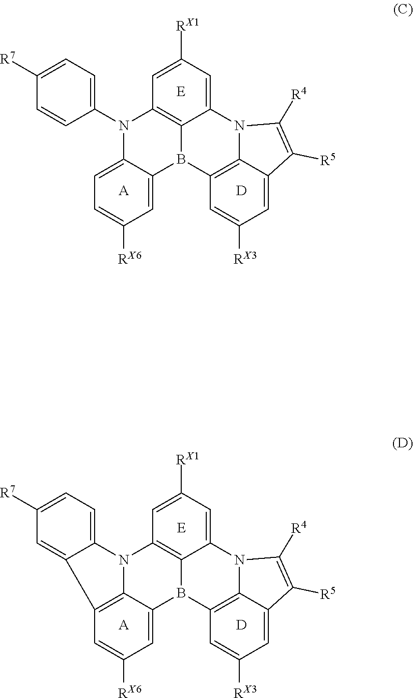

Preferred compounds (C) and (D) are mentioned in the following table:

| R^{X6} | R⁷ | R^{X1} | R^{X3} | R⁴ | R⁵ |
|---|---|---|---|---|---|
| H | H | H | tBu | p-tBuC₆H₄ | p-tBuC₆H₄ |
| tBu | tBu | H | tBu | p-tBuC₆H₄ | p-tBuC₆H₄ |
| tBu | tBu | H | tBu | Me | Me |
| H | H | H | tBu | Me | Me |
| H | H | H | tBu | Me | H |
| H | H | H | tBu | H | Me |
| tBu | tBu | H | tBu | H | Me |
| tBu | tBu | H | tBu | H | tBu |
| tBu | tBu | H | tBu | Me | H |
| tBu | tBu | H | tBu | tBu | H |
| H | H | H | tBu | H | p-tBuC₆H₄ |
| tBu | tBu | H | tBu | H | p-tBuC₆H₄ |
| H | H | H | tBu | p-tBuC₆H₄ | H |
| tBu | tBu | H | tBu | p-tBuC₆H₄ | H |
| H | H | Me | tBu | p-tBuC₆H₄ | p-tBuC₆H₄ |
| tBu | tBu | Me | tBu | p-tBuC₆H₄ | p-tBuC₆H₄ |
| tBu | tBu | Me | tBu | Me | Me |
| H | H | Me | tBu | Me | Me |
| H | H | Me | tBu | Me | H |
| H | H | Me | tBu | H | Me |
| tBu | tBu | Me | tBu | H | Me |
| tBu | tBu | Me | tBu | H | tBu |
| tBu | tBu | Me | tBu | Me | H |
| tBu | tBu | Me | tBu | tBu | H |
| H | H | Me | tBu | H | p-tBuC₆H₄ |
| tBu | tBu | Me | tBu | H | p-tBuC₆H₄ |
| H | H | Me | tBu | p-tBuC₆H₄ | H |
| tBu | tBu | Me | tBu | p-tBuC₆H₄ | H |
| H | H | H | tBu | Ph | Ph |
| tBu | tBu | H | tBu | Ph | Ph |
| H | H | H | tBu | H | Ph |
| tBu | tBu | H | tBu | H | Ph |
| H | H | H | tBu | Ph | H |
| tBu | tBu | H | tBu | Ph | H |
| H | H | Me | tBu | Ph | Ph |
| tBu | tBu | Me | tBu | Ph | Ph |
| H | H | Me | tBu | H | Ph |
| tBu | tBu | Me | tBu | H | Ph |
| H | H | Me | tBu | Ph | H |
| tBu | tBu | Me | tBu | Ph | H |

Examples for compounds of class 1 are compounds of the following formula (E), wherein the residues R^{X1}, R^{X3}, R^{X6} and R⁷ are defined above.

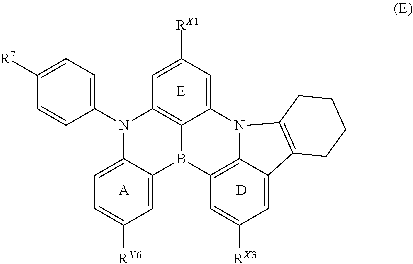

Preferred compounds (E) are mentioned in the following table:

| R^{X6} | R⁷ | R^{X1} | R^{X3} |
|---|---|---|---|
| tBu | tBu | H | H |
| H | H | H | H |
| tBu | tBu | Me | H |
| H | H | Me | H |
| tBu | tBu | H | tBu |
| H | H | H | tBu |
| tBu | tBu | Me | tBu |
| H | H | Me | tBu |
| tBu | tBu | CD₃ | tBu |
| H | H | CD₃ | tBu |

Examples for compounds of class 1 are compounds of the following formulae (A), (B) and (F), wherein the residues R⁴, R⁵, R^{X1}, R^{X6} and R⁷ are defined above.

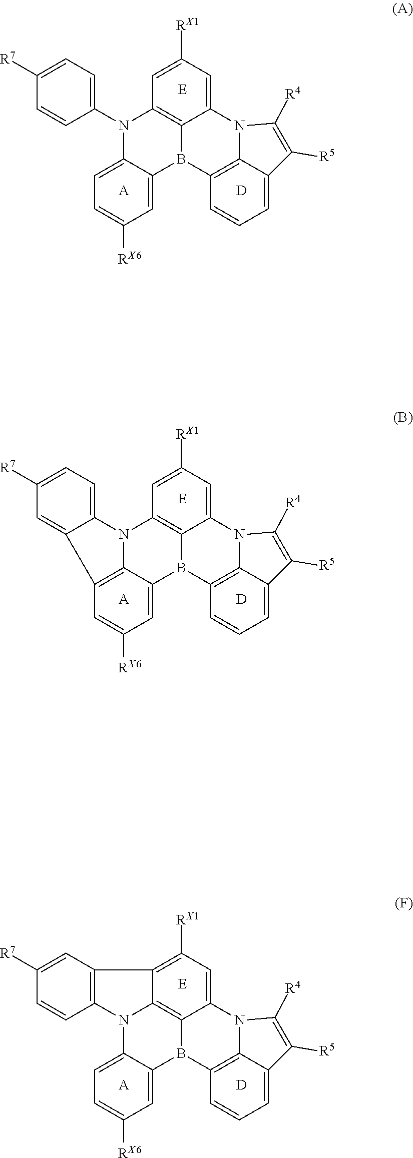

Preferred compounds (A), (B) and (F) are mentioned in the following table, wherein groups 5 and 7b are defined as follows:

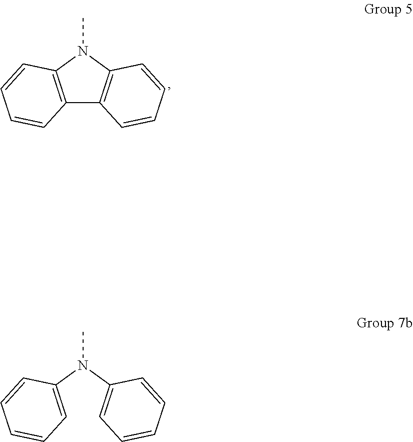

| R^{X6} | R⁷ | R^{X1} | R⁴ | R⁵ |
|---|---|---|---|---|
| H | H | Me | Ph | Ph |
| tBu | tBu | Me | Ph | Ph |
| tBu | tBu | Me | Me | Me |
| H | H | Me | Me | Me |
| H | H | Me | Me | H |
| H | H | Me | H | Me |
| tBu | tBu | Me | H | Me |
| tBu | tBu | Me | H | tBu |
| tBu | tBu | Me | Me | H |
| tBu | tBu | Me | tBu | H |
| H | H | Me | H | Ph |
| tBu | tBu | Me | H | Ph |
| H | H | Me | Ph | H |
| tBu | tBu | Me | Ph | H |
| H | H | CD₃ | Ph | Ph |
| tBu | tBu | CD₃ | Ph | Ph |
| tBu | tBu | CD₃ | Me | Me |
| H | H | CD₃ | Me | Me |
| H | H | CD₃ | Me | H |
| H | H | CD₃ | H | Me |
| tBu | tBu | CD₃ | H | Me |
| tBu | tBu | CD₃ | H | tBu |
| tBu | tBu | CD₃ | Me | H |
| tBu | tBu | CD₃ | tBu | H |
| H | H | CD₃ | H | Ph |
| tBu | tBu | CD₃ | H | Ph |
| H | H | CD₃ | Ph | H |
| tBu | tBu | CD₃ | Ph | H |
| H | H | Group7b | Ph | Ph |
| tBu | tBu | Group7b | Ph | Ph |
| tBu | tBu | Group7b | Me | Me |
| H | H | Group7b | Me | Me |
| H | H | Group7b | Me | H |
| H | H | Group7b | H | Me |
| tBu | tBu | Group7b | H | Me |
| tBu | tBu | Group7b | H | tBu |
| tBu | tBu | Group7b | Me | H |
| tBu | tBu | Group7b | tBu | H |
| H | H | Group7b | H | Ph |
| tBu | tBu | Group7b | H | Ph |
| H | H | Group7b | Ph | H |
| tBu | tBu | Group7b | Ph | H |
| H | H | Group5 | Ph | Ph |
| tBu | tBu | Group5 | Ph | Ph |
| tBu | tBu | Group5 | Me | Me |
| H | H | Group5 | Me | Me |
| H | H | Group5 | Me | H |
| H | H | Group5 | H | Me |
| tBu | tBu | Group5 | H | Me |
| tBu | tBu | Group5 | H | tBu |
| tBu | tBu | Group5 | Me | H |
| tBu | tBu | Group5 | tBu | H |
| H | H | Group5 | H | Ph |
| tBu | tBu | Group5 | H | Ph |
| H | H | Group5 | Ph | H |
| tBu | tBu | Group5 | Ph | H |
| H | H | OMe | Ph | Ph |
| tBu | tBu | OMe | Ph | Ph |
| tBu | tBu | OMe | Me | Me |
| H | H | OMe | Me | Me |
| H | H | OMe | Me | H |
| H | H | OMe | H | Me |
| tBu | tBu | OMe | H | Me |
| tBu | tBu | OMe | H | tBu |
| tBu | tBu | OMe | Me | H |
| tBu | tBu | OMe | tBu | H |
| H | H | OMe | H | Ph |
| tBu | tBu | OMe | H | Ph |
| H | H | OMe | Ph | H |
| tBu | tBu | OMe | Ph | H |
| H | H | OPh | Ph | Ph |
| tBu | tBu | OPh | Ph | Ph |
| tBu | tBu | OPh | Me | Me |
| H | H | OPh | Me | Me |
| H | H | OPh | Me | H |
| H | H | OPh | H | Me |
| tBu | tBu | OPh | H | Me |
| tBu | tBu | OPh | H | tBu |
| tBu | tBu | OPh | Me | H |
| tBu | tBu | OPh | tBu | H |
| H | H | OPh | H | Ph |
| tBu | tBu | OPh | H | Ph |
| H | H | OPh | Ph | H |
| tBu | tBu | OPh | Ph | H |

Examples for compounds of class 1 are compounds of the following formula (G), wherein the residues R⁴, R⁵, R, R^{X1}, R^{X2}, R^{X3}, R^{X5} and R^{X7} are defined above.

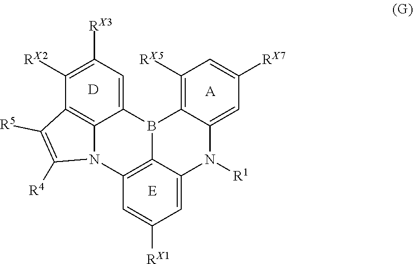

Preferred compounds (G) are mentioned in the following table, wherein Group 1, 2, 3 and 4 are defined as follows:

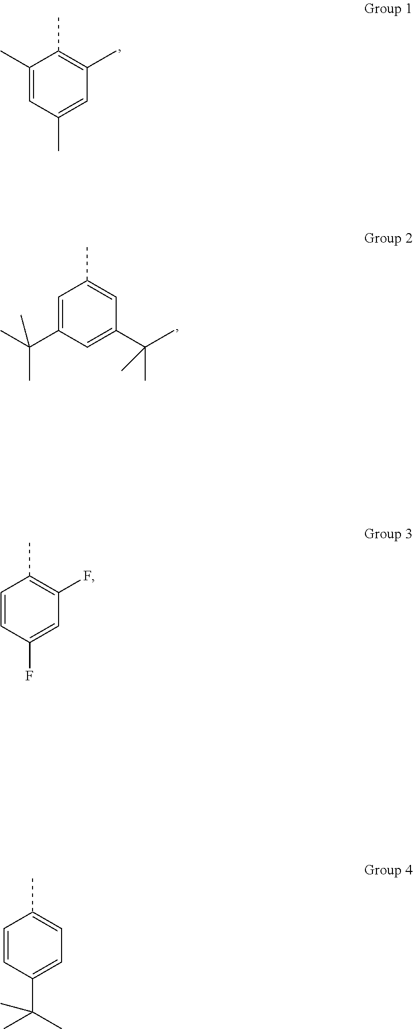

| R⁴ | R⁵ | R^{X5} | R^{X7} | R¹ | R^{X1} | R^{X3} | R^{X2} |
|---|---|---|---|---|---|---|---|
| Group4 | Group4 | F | F | Group3 | H | H | H |
| Ph | Ph | F | F | Group3 | H | H | H |
| Me | Group1 | H | tBu | Group4 | H | H | H |
| Et | Group1 | H | tBu | Group4 | H | H | H |
| Pr | Group1 | H | tBu | Group4 | H | H | H |
| Me | Group1 | F | F | Group3 | H | H | H |
| Me | Group1 | F | F | Group1 | H | H | H |
| Group4 | Group4 | F | F | Group3 | H | tBu | H |
| Ph | Ph | F | F | Group3 | H | tBu | H |
| Me | Group1 | H | tBu | Group4 | H | tBu | H |
| Et | Group1 | H | tBu | Group4 | H | tBu | H |
| Pr | Group1 | H | tBu | Group4 | H | tBu | H |
| Me | Group1 | F | F | Group3 | H | tBu | H |
| Me | Group1 | H | tBu | Group4 | H | H | F |
| Group4 | Group4 | F | F | Group3 | Me | H | H |
| Ph | Ph | F | F | Group3 | Me | H | H |
| Me | Group1 | H | tBu | Group4 | Me | H | H |
| Et | Group1 | H | tBu | Group4 | Me | H | H |
| Pr | Group1 | H | tBu | Group4 | Me | H | H |
| Me | Group1 | F | F | Group3 | Me | H | H |
| Me | Group1 | F | F | Group1 | Me | H | H |
| Group4 | Group4 | F | F | Group3 | Me | tBu | H |
| Ph | Ph | F | F | Group3 | Me | tBu | H |
| Me | Group1 | H | tBu | Group4 | Me | tBu | H |
| Et | Group1 | H | tBu | Group4 | Me | tBu | H |
| Pr | Group1 | H | tBu | Group4 | Me | tBu | H |
| Me | Group1 | F | F | Group3 | Me | tBu | H |
| Me | Group1 | H | tBu | Group4 | Me | H | F |
| Group4 | Group4 | F | F | Group3 | Me | H | H |
| Ph | Ph | F | F | Group3 | Me | H | H |
| Me | Group1 | H | tBu | Group4 | Me | H | H |
| Et | Group1 | H | tBu | Group4 | Me | H | H |
| Pr | Group1 | H | tBu | Group4 | Me | H | H |
| Me | Group1 | F | F | Group3 | Me | H | H |
| Me | Group1 | F | F | Group1 | Me | H | H |
| Group4 | Group4 | F | F | Group3 | Me | tBu | H |
| Ph | Ph | F | F | Group3 | Me | tBu | H |
| Me | Group1 | H | tBu | Group4 | Me | tBu | H |
| Et | Group1 | H | tBu | Group4 | Me | tBu | H |
| Pr | Group1 | H | tBu | Group4 | Me | tBu | H |
| Me | Group1 | F | F | Group3 | Me | tBu | H |
| Me | Group1 | H | tBu | Group4 | Me | H | F |
| Ph | Ph | H | OPh | Group2 | Me | H | H |
| Me | Group1 | H | OPh | Group2 | Me | H | H |
| Ph | Ph | H | OMe | Group2 | Me | H | H |
| Me | Group1 | H | OMe | Group2 | Me | H | H |
| Ph | Ph | H | OPh | Group2 | Me | H | tBu |
| Me | Group1 | H | OPh | Group2 | Me | H | tBu |
| Ph | Ph | H | OMe | Group2 | Me | H | tBu |
| Me | Group1 | H | OMe | Group2 | Me | H | tBu |
| Ph | Ph | H | OPh | Group2 | H | H | H |
| Me | Group1 | H | OPh | Group2 | H | H | H |
| Ph | Ph | H | OMe | Group2 | H | H | H |
| Me | Group1 | H | OMe | Group2 | H | H | H |

Further preferred compounds (G) are mentioned in the following table, wherein Group 4, 5, 5b, 6, 6b, 7, 8 and 8b are defined as follows:

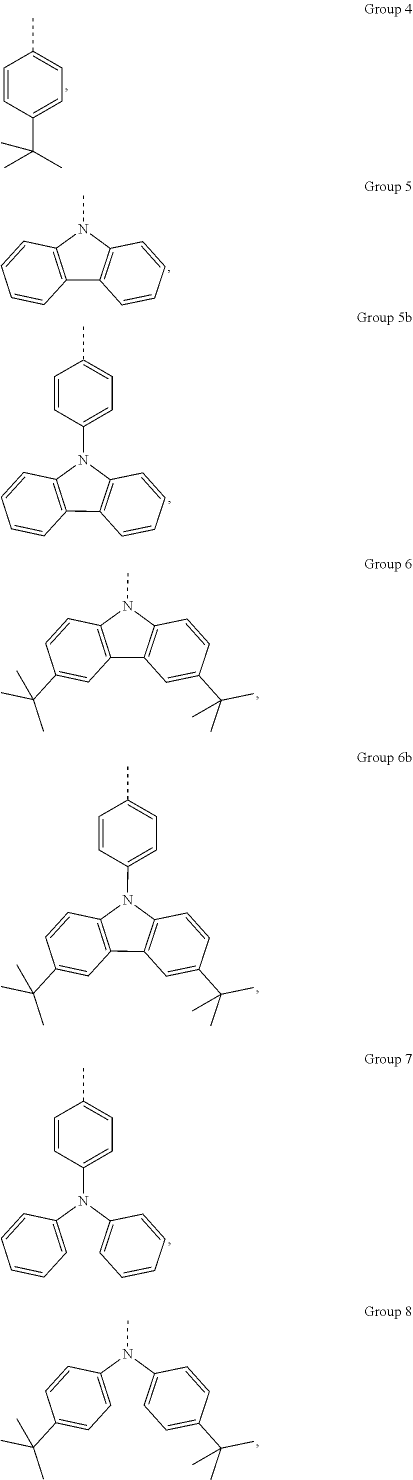

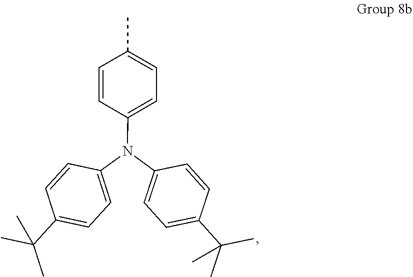

| R⁴ | R⁵ | R^{X5} | R^{X7} | R¹ | R^{X1} | R^{X3} | R^{X2} |
|---|---|---|---|---|---|---|---|
| Ph | Ph | H | Group5 | Group5b | Me | H | H |
| Ph | Ph | H | Group5 | Group5b | H | H | H |
| Group4 | Group4 | H | Group5 | Group5b | H | H | H |
| Ph | Ph | H | Group6 | Group6b | Me | H | H |
| Ph | Ph | H | Group6 | Group6b | H | H | H |
| Group4 | Group4 | H | Group6 | Group6b | H | H | H |
| Ph | Ph | H | Group7 | Group7 | Me | H | H |
| Ph | Ph | H | Group7 | Group7 | H | H | H |
| Group4 | Group4 | H | Group7 | Group7 | H | H | H |
| Ph | Ph | H | Group8 | Group8b | Me | H | H |
| Ph | Ph | H | Group8 | Group8b | H | H | H |
| Group4 | Group4 | H | Group8 | Group8b | H | H | H |

Further preferred compounds (G)

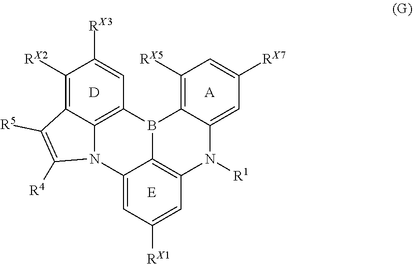

are mentioned in the following table, wherein Group 10, 2, 3 and 4 are defined as follows:

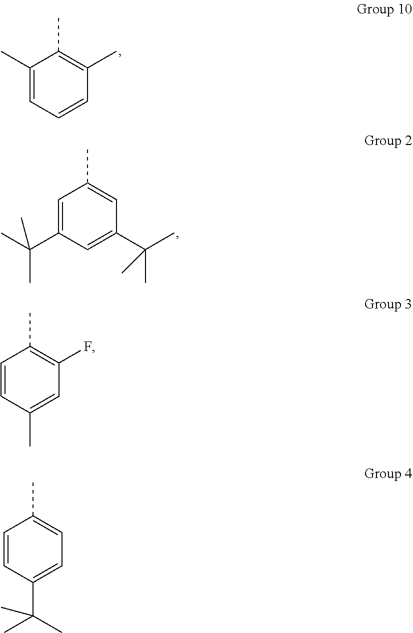

| R⁴ | R⁵ | R^{X5} | R^{X7} | R¹ | R^{X1} | R^{X3} | R^{X2} |
|---|---|---|---|---|---|---|---|
| Me | Group10 | H | tBu | Group4 | H | H | H |
| Et | Group10 | H | tBu | Group4 | H | H | H |
| Pr | Group10 | H | tBu | Group4 | H | H | H |
| Me | Group10 | F | F | Group3 | H | H | H |
| Me | Group10 | F | F | Group10 | H | H | H |
| Me | Group10 | H | tBu | Group4 | H | tBu | H |
| Et | Group10 | H | tBu | Group4 | H | tBu | H |
| Pr | Group10 | H | tBu | Group4 | H | tBu | H |
| Me | Group10 | F | F | Group3 | H | tBu | H |
| Me | Group10 | H | tBu | Group4 | H | H | F |
| Group4 | Group4 | F | F | Group2 | Me | H | H |
| Group4 | Group4 | F | F | Group2 | | H | H |
| Group4 | Group4 | F | F | Group2 | Me | tBu | H |
| Me | Group10 | H | tBu | Group4 | Me | H | H |
| Et | Group10 | H | tBu | Group4 | Me | H | H |
| Pr | Group10 | H | tBu | Group4 | Me | H | H |
| Me | Group10 | F | F | Group3 | Me | H | H |
| Me | Group10 | F | F | Group10 | Me | H | H |
| Me | Group10 | H | tBu | Group4 | Me | tBu | H |
| Et | Group10 | H | tBu | Group4 | Me | tBu | H |
| Pr | Group10 | H | tBu | Group4 | Me | tBu | H |
| Me | Group10 | F | F | Group3 | Me | tBu | H |
| Me | Group10 | H | tBu | Group4 | Me | H | F |
| Me | Group10 | H | tBu | Group4 | Me | H | H |
| Et | Group10 | H | tBu | Group4 | Me | H | H |
| Pr | Group10 | H | tBu | Group4 | Me | H | H |
| Me | Group10 | F | F | Group3 | Me | H | H |
| Me | Group10 | F | F | Group10 | Me | H | H |
| Me | Group10 | H | tBu | Group4 | Me | tBu | H |
| Et | Group10 | H | tBu | Group4 | Me | tBu | H |
| Pr | Group10 | H | tBu | Group4 | Me | tBu | H |
| Me | Group10 | F | F | Group3 | Me | tBu | H |
| Me | Group10 | H | tBu | Group4 | Me | H | F |
| Me | Group10 | H | OPh | Group2 | Me | H | H |
| Me | Group10 | H | OMe | Group2 | Me | H | H |
| Me | Group10 | H | OPh | Group2 | Me | H | tBu |
| Me | Group10 | H | OMe | Group2 | Me | H | tBu |
| Me | Group10 | H | OPh | Group2 | H | H | H |
| Me | Group10 | H | OMe | Group2 | H | H | H |

Examples for compounds of class 1 are compounds of the following formula (H), wherein the residues R⁴, R⁵, R⁷, R^{X1}, R^{X2}, R^{X3}, R^{X5} and R^{X7} are defined above.

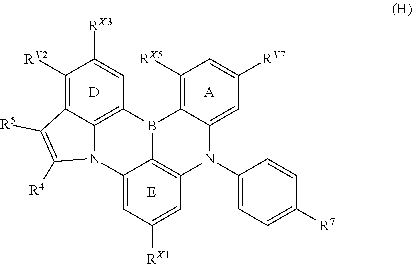

Preferred compounds (H) are mentioned in the following table, wherein Group 4, 5, 6, 7b and 8 are defined as follows:

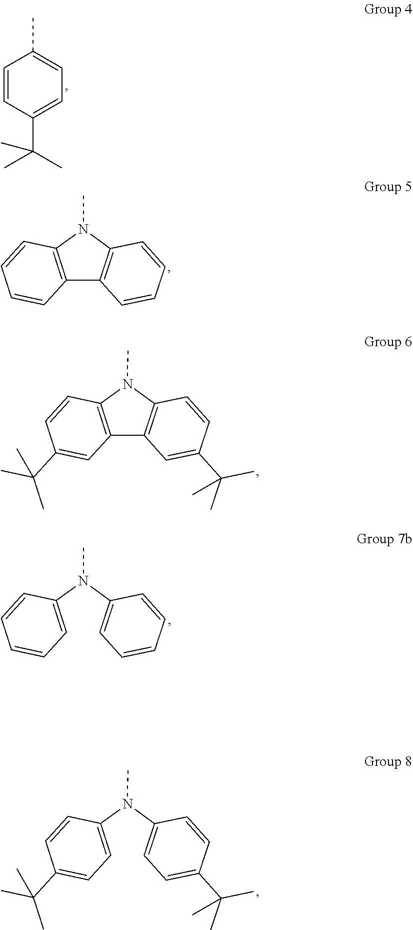

| R⁴ | R⁵ | R^{X5} | R^{X7} | R⁷ | R^{X1} | R^{X3} | R^{X2} |
|---|---|---|---|---|---|---|---|
| Ph | Ph | H | Group5 | H | Me | H | H |
| Ph | Ph | H | Group5 | H | H | H | H |
| Group4 | Group4 | H | Group5 | H | H | H | H |
| Group4 | Group4 | H | Group5 | H | Me | H | H |
| Ph | Ph | H | Group6 | H | Me | H | H |
| Ph | Ph | H | Group6 | H | H | H | H |
| Group4 | Group4 | H | Group6 | H | H | H | H |
| Group4 | Group4 | H | Group6 | H | Me | H | H |
| Ph | Ph | H | Group7b | H | Me | H | H |
| Ph | Ph | H | Group7b | H | H | H | H |
| Group4 | Group4 | H | Group7b | H | H | H | H |
| Group4 | Group4 | H | Group7b | H | Me | H | H |
| Ph | Ph | H | Group8 | H | Me | H | H |
| Ph | Ph | H | Group8 | H | H | H | H |
| Group4 | Group4 | H | Group8 | H | H | H | H |
| Group4 | Group4 | H | Group8 | H | Me | H | H |
| Ph | Ph | H | Group5 | tBu | Me | H | H |
| Ph | Ph | H | Group5 | tBu | H | H | H |
| Group4 | Group4 | H | Group5 | tBu | H | H | H |
| Group4 | Group4 | H | Group5 | tBu | Me | H | H |
| Ph | Ph | H | Group6 | tBu | Me | H | H |
| Ph | Ph | H | Group6 | tBu | H | H | H |
| Group4 | Group4 | H | Group6 | tBu | H | H | H |
| Group4 | Group4 | H | Group6 | tBu | Me | H | H |
| Ph | Ph | H | Group7b | tBu | Me | H | H |
| Ph | Ph | H | Group7b | tBu | H | H | H |
| Group4 | Group4 | H | Group7b | tBu | H | H | H |
| Group4 | Group4 | H | Group7b | tBu | Me | H | H |
| Ph | Ph | H | Group8 | tBu | Me | H | H |
| Ph | Ph | H | Group8 | tBu | H | H | H |
| Group4 | Group4 | H | Group8 | tBu | H | H | H |
| Group4 | Group4 | H | Group8 | tBu | Me | H | H |

Further preferred compounds (H)

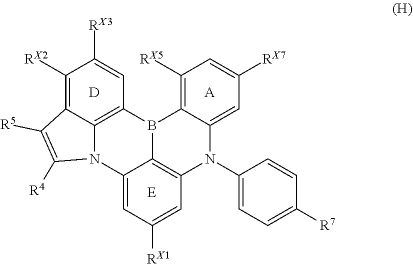

are mentioned in the following table, wherein Group 10, 1 and 4 are defined as follows:

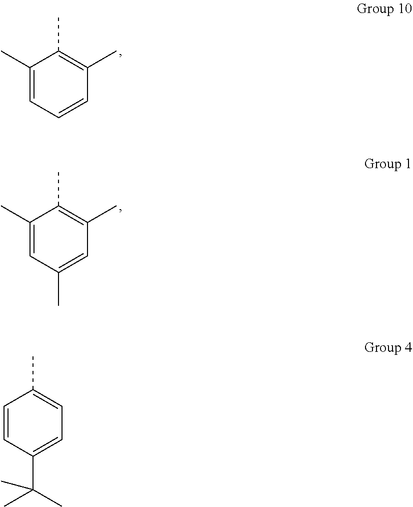

| R⁴ | R⁵ | R^{X5} | R^{X7} | R⁷ | R^{X1} | R^{X3} | R^{X2} |
|---|---|---|---|---|---|---|---|
| Me | group1 | H | tBu | tBu | F | H | H |
| Me | group10 | H | tBu | tBu | F | H | H |
| Me | group4 | H | tBu | tBu | F | H | H |
| Me | Ph | H | tBu | tBu | F | H | H |
| Ph | group1 | H | tBu | tBu | F | H | H |
| Ph | group10 | H | tBu | tBu | F | H | H |
| Ph | group4 | H | tBu | tBu | F | H | H |
| Ph | Ph | H | tBu | tBu | F | H | H |
| tBu | group1 | H | tBu | tBu | F | H | H |
| tBu | group10 | H | tBu | tBu | F | H | H |
| tBu | group4 | H | tBu | tBu | F | H | H |
| tBu | Ph | H | tBu | tBu | F | H | H |
| Me | group1 | H | tBu | tBu | F | H | tBu |
| Me | group10 | H | tBu | tBu | F | H | tBu |
| Me | group4 | H | tBu | tBu | F | H | tBu |
| Me | Ph | H | tBu | tBu | F | H | tBu |
| Ph | group1 | H | tBu | tBu | F | H | tBu |
| Ph | group10 | H | tBu | tBu | F | H | tBu |
| Ph | group4 | H | tBu | tBu | F | H | tBu |
| Ph | Ph | H | tBu | tBu | F | H | tBu |
| tBu | group1 | H | tBu | tBu | F | H | tBu |
| tBu | group10 | H | tBu | tBu | F | H | tBu |
| tBu | group4 | H | tBu | tBu | F | H | tBu |
| tBu | Ph | H | tBu | tBu | F | H | tBu |
| Me | group1 | H | H | H | F | H | H |
| Me | group10 | H | H | H | F | H | H |
| Me | group4 | H | H | H | F | H | H |
| Me | Ph | H | H | H | F | H | H |
| Ph | group1 | H | H | H | F | H | H |
| Ph | group10 | H | H | H | F | H | H |
| Ph | group4 | H | H | H | F | H | H |
| Ph | Ph | H | H | H | F | H | H |
| tBu | group1 | H | H | H | F | H | H |
| tBu | group10 | H | H | H | F | H | H |
| tBu | group4 | H | H | H | F | H | H |
| tBu | Ph | H | H | H | F | H | H |

Examples for compounds of class 1 are compounds of the following formula (I*), wherein the residues R⁴, R⁵, R⁷, R^{X1}, R^{X2}, R^{X3}, R^{X5} and R^{X7} are defined above.

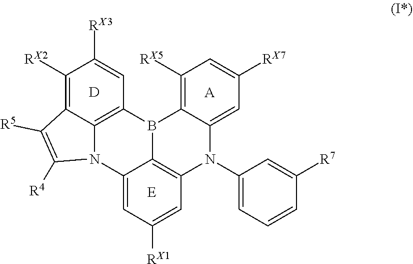

Preferred compounds (I*) are mentioned in the following table, wherein Group 1 and 4 are defined as follows:

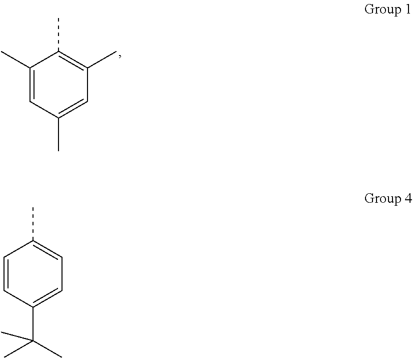

| R⁴ | R⁵ | R^{X5} | R^{X7} | R⁷ | R^{X1} | R^{X3} | R^{X2} |
|---|---|---|---|---|---|---|---|
| Ph | Ph | H | Ph | Ph | Me | H | H |
| Ph | Ph | H | Ph | Ph | H | H | H |
| Group4 | Group4 | H | Ph | Ph | Me | H | H |
| Group4 | Group4 | H | Ph | Ph | H | H | H |
| Group1 | Group1 | H | Ph | Ph | Me | H | H |
| Group1 | Group1 | H | Ph | Ph | H | H | H |

The compounds of formula

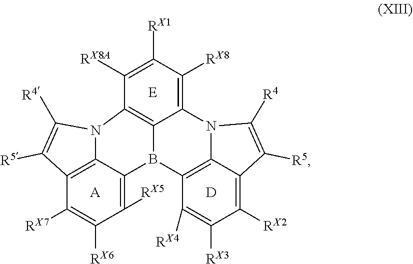

### Class 2

In the compounds of class 2 (formula (XIII)), the groups, residues and indices R⁴, R⁵, R^{4′}, R^{5′}, R^{X1}, R^{X2}, R^{X3}, R^{X4}, R^{X5}, R^{X6}, R^{X7}, R^{X8} and R^{X8A} are defined above.

In the case that R^{X6} and R^{X7}, R^{X5} and R^{X6}, R^{X4} and R^{X3}, R^{X2} and R^{X3}, R^{X8} and R^{X1}, and/or R^{X1} and R^{X8A} in the compounds of class 2 may form together an unsubstituted or substituted ring, the following rings (a) and (b) are formed:

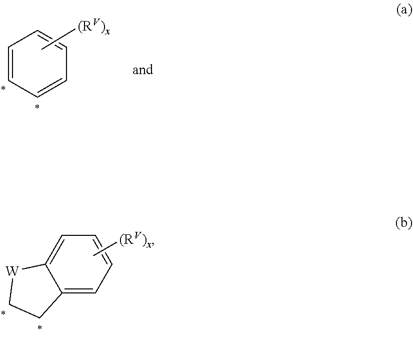

wherein

R^{V} represents H, a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group;

x represents 0, 1, 2, 3 or 4, preferably 0, 1 or 2, more preferably 0; and

W represents CR′″₂, O, S or NR^{IV};

R′″ represents a C₁-C₂₅ alkyl group, preferably a C₁-C₈ alkyl group, more preferably a C₁-C₄ alkyl group;

R^{IV} represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group; and

each * represents a point of attachment to ring A, D or E.

Most preferably, R⁴, R⁵, R^{4′} and R^{5′} in the compounds of class 2 each independently represents methyl, ethyl, iso-propyl, sec-propyl, n-butyl, —C(Me)₂C₂H₅, unsubstituted phenyl, p-tert-butyl-phenyl, mesityl, xylyl, O-methyl-phenyl, m-(^{t}butyl)₂-phenyl or unsubstituted or substituted biphenyl, preferably unsubstituted biphenyl; or

R⁴ and R⁵ may form together an unsubstituted or substituted cyclohexene ring, preferably an unsubstituted cyclohexene ring, and/or

R^{4′} and R^{5′} may form together an unsubstituted or substituted phenyl ring or an unsubstituted or substituted cyclohexene ring, preferably an unsubstituted phenyl ring or an unsubstituted cyclohexene ring.

An example for compounds of class 2, wherein R⁴ and R⁵ may form together an unsubstituted cyclohexene ring is the following compound:

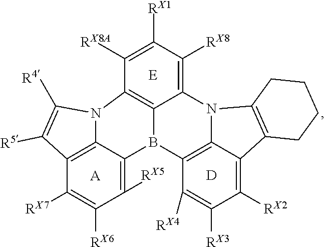

wherein the groups and residues are defined above and below.

Examples for compounds of class 2, wherein R^{4′} and R^{5′} may form together an unsubstituted phenyl ring or an unsubstituted cyclohexene ring are the following compounds:

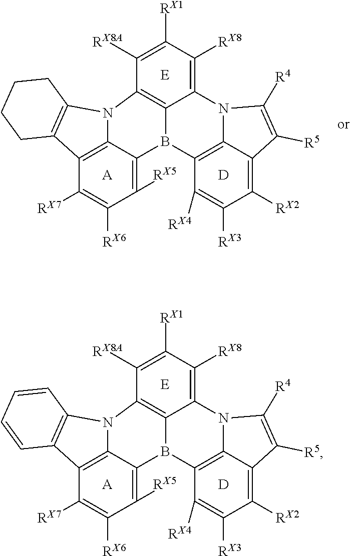

wherein the groups and residues are defined above and below.

Most preferably, R^{X5}, R^{X6} and R^{X7} and R^{X2}, R^{X3} and R^{X4} in the compounds of class 2 each independently represents H, methyl, ethyl, iso-propyl, sec-propyl, n-butyl, tert-butyl, —C(Me)₂C₂H₅, F, unsubstituted phenyl, p-tert-butyl-phenyl, mesityl, xylyl, O-methyl-phenyl or unsubstituted or substituted biphenyl, preferably unsubstituted biphenyl.

Most preferably, R^{X1}, R^{X8} and R^{X8A} in the compounds of class 2 each independently represents H, methyl, ethyl, n-butyl, unsubstituted phenyl, —O-phenyl, —NPh₂, —N-carbazolyl or —N(C₆H₅^{t}Bu)₂; or

R^{X8} and R⁴ and/or R^{X8A} and R^{4′}, may form together a ring, wherein the ring is formed via a single bond, via a C₁-C₃ alkyl group which is optionally substituted by a C₁-C₂₅ alkyl group, preferably by a C₁-C₈ alkyl group, more preferably by a C₁-C₄ alkyl group, via an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group, via an unsubstituted or substituted C₂ alkenyl group, via a group NR′, via O, via a group POOR′ or via a unsubstituted or substituted P—C₆-C₃₀ aryl group, preferably unsubstituted or substituted P—C₆-C₁₀ aryl group, more preferably unsubstituted or substituted P—C₆ aryl group, most preferably unsubstituted P—C₆ aryl group; preferably via an unsubstituted or substituted C₆ aryl group,

wherein

R′ represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group.

Examples for compounds of class 2, wherein R^{X8} and R⁴ and/or R^{X8A} and R^{4′} form together a ring are the following compounds:

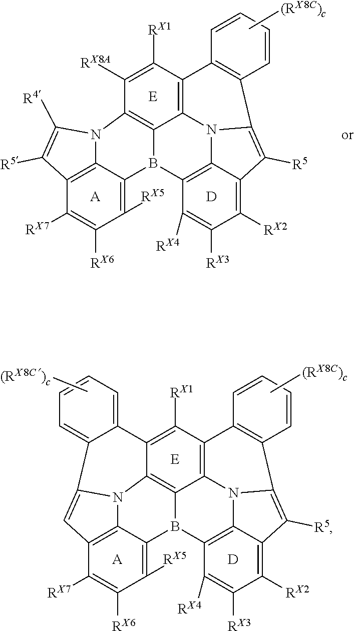

wherein

R^{X8C} and R^{X8C′} each independently represents H, methyl, ethyl, iso-propyl, sec-propyl, n-butyl, tert-butyl, —C(Me)₂C₂H₅, F, unsubstituted phenyl, p-tert-butyl-phenyl, mesityl, xylyl, o-methyl-phenyl or unsubstituted or substituted biphenyl, preferably unsubstituted biphenyl, and

c represents 0, 1, 2, 3 or 4, preferably 0, 1 or 2.

Most preferably, R^{X4} and R^{X5} in the compounds of class 2 (formula (XIII)) are H, and further most preferably R^{X4}, R^{X5}, R^{X8} and R^{X8A} are H.

Examples for compounds of class 2 are compounds of the following formula J, wherein the residues R⁴, R⁵, R^{4′}, R^{5′}, R^{X1}, R^{X2}, R^{X3}, R^{X6} and R^{X7} are defined above.

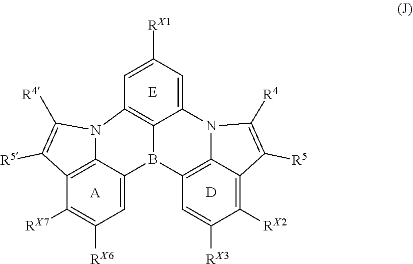

Preferred compounds (J) are mentioned in the following table, wherein Groups 1, 2 and 4 are defined as follows:

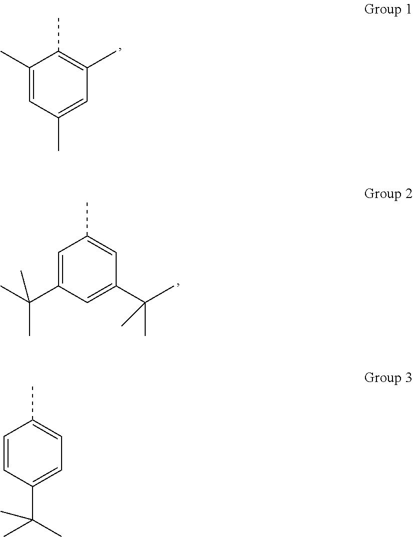

| R⁴, R^{4′} | R⁵, R^{5′} | R^{X1} | R^{X3}, R^{X6} | RX², R^{X7} |
|---|---|---|---|---|
| Me | Me | H | H | H |
| Me | Group1 | H | H | H |
| Me | Group2 | H | H | H |
| Me | Group4 | H | H | H |
| Ph | Ph | H | H | H |
| Group1 | Group1 | H | H | H |
| Group4 | Group4 | H | H | H |
| Me | Me | Me | H | H |
| Me | Group1 | Me | H | H |
| Me | Group2 | Me | H | H |
| Me | Group4 | Me | H | H |
| Ph | Ph | Me | H | H |
| Group1 | Group1 | Me | H | H |
| Me | Me | H | tBu | H |
| Me | Group1 | H | tBu | H |
| Me | Group2 | H | tBu | H |
| Me | Group4 | H | tBu | H |
| Ph | Ph | H | tBu | H |
| Group1 | Group1 | H | tBu | H |
| Group4 | Group4 | H | tBu | H |
| Me | Me | Me | tBu | H |
| Me | Group1 | Me | tBu | H |
| Me | Group2 | Me | tBu | H |
| Me | Group4 | Me | tBu | H |
| Ph | Ph | Me | tBu | H |
| Group1 | Group1 | Me | tBu | H |
| Me | Me | H | H | F |
| Me | Group1 | H | H | F |
| Me | Group2 | H | H | F |
| Me | Group4 | H | H | F |
| Ph | Ph | H | H | F |
| Group1 | Group1 | H | H | F |
| Group4 | Group4 | H | H | F |
| Me | Me | Me | H | F |
| Me | Group1 | Me | H | F |
| Me | Group2 | Me | H | F |
| Me | Group4 | Me | H | F |
| Ph | Ph | Me | H | F |
| Group1 | Group1 | Me | H | F |

Further preferred compounds (J) are mentioned in the following table, wherein Groups 10, 2 and 4 are defined as follows:

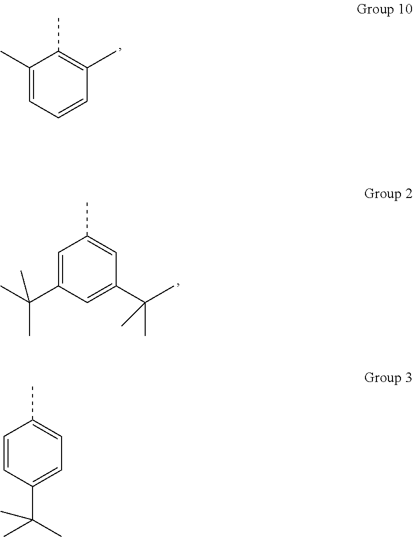

| R⁴, R^{4′} | R⁵, R^{5′} | R^{X1} | R^{X3}, R^{X6} | R^{X2}, R^{X7} |
|---|---|---|---|---|
| Me | Group 10 | H | H | H |
| Group 10 | Group 10 | H | H | H |
| Group 10 | Group 10 | Me | H | H |
| Me | Group 10 | H | tBu | H |
| Group 10 | Group 10 | H | tBu | H |
| Me | Group 10 | Me | tBu | H |
| Group 1 | Group 1 | Me | tBu | H |
| Me | Group 1 | H | H | F |
| Group 1 | Group 1 | H | H | F |
| Me | Group 1 | Me | H | F |
| Group 10 | Group 10 | Me | H | F |

The compounds of formula

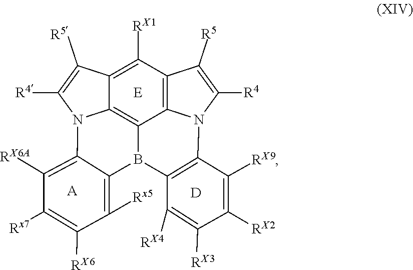

### Class 3

In the compounds of class 3 (formula (XIV)), the groups, residues and indices R⁴, R⁵, R^{4′}, R^{5′}, R^{X1}, R^{X2}, R^{X3}, R^{X4}, R^{X5}, R^{X6}, R^{X7}, R^{X6A} and R^{X9} are defined above.

In the case that R^{X6A} and R^{X7}, R^{X6} and R^{X7}, R^{X5} and R^{X6}, R^{X4} and R^{X3}, R^{X2} and R^{X3}, and/or R^{X2} and R^{X9} in the compounds of class 3 may form together an unsubstituted or substituted ring, the following rings (a) and (b) are formed:

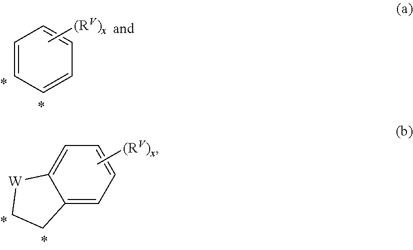

wherein

R^{V} represents H, a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group;

x represents 0, 1, 2, 3 or 4, preferably 0, 1 or 2, more preferably 0; and

W represents CR′″₂, O, S or NR^{IV};

R′″ represents a C₁-C₂₅ alkyl group, preferably a C₁-C₈ alkyl group, more preferably a C₁-C₄ alkyl group;

R^{IV} represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group; and

each * represents a point of attachment to ring A or D.

Most preferably, R^{X1} in the compounds of class 3 (formula (XIV)) is H. Further most preferably, R^{X1}, R^{X4} and R^{X5} in the compounds of class 3 are H.

Most preferably, R⁴, R⁵, R^{4′} and R^{5′} in the compounds of class 3 each independently represents methyl, ethyl, iso-propyl, sec-propyl, n-butyl, —C(Me)₂C₂H₅, unsubstituted phenyl, p-tert-butyl-phenyl, mesityl, xylyl, O-methyl-phenyl, m-(^{t}butyl)₂-phenyl or substituted or unsubstituted biphenyl, preferably unsubstituted biphenyl; or

R⁴ and R⁵ may form together an unsubstituted or substituted cyclohexene ring, preferably an unsubstituted cyclohexene ring, and/or

R^{4′} and R^{5′} may form together an unsubstituted or substituted phenyl ring or an unsubstituted or substituted cyclohexene ring, preferably an unsubstituted phenyl ring or an unsubstituted cyclohexene ring.

Examples for compounds of class 3, wherein R⁴ and R⁵ may form together an unsubstituted cyclohexene ring is the following compound:

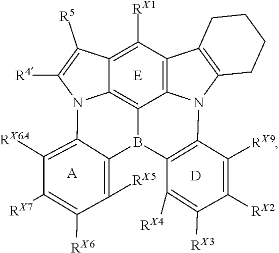

wherein the groups and residues are defined above and below.

An example for compounds of class 3, wherein R^{4′} and R^{5′} may form together an unsubstituted phenyl ring or an unsubstituted cyclohexene ring are the following compounds:

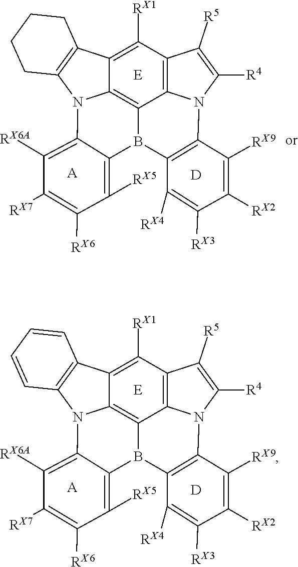

wherein the groups and residues are defined above and below.

Most preferably, R^{X6A}, R^{X7}, R^{X6}, R^{X3}, R^{X2} and R^{X9} each independently represents H, methyl, ethyl, iso-propyl, sec-propyl, n-butyl, tert-butyl, —C(Me)₂Et, unsubstituted phenyl, p-tert-butyl-phenyl, mesityl, xylyl, O-methyl-phenyl or substituted or unsubstituted biphenyl, preferably unsubstituted biphenyl.

Examples for compounds of class 3 are compounds of the following formula (K), wherein the residues R⁴, R⁵, R^{4′}, R^{5′}, R^{X3} and R^{X6} are defined above.

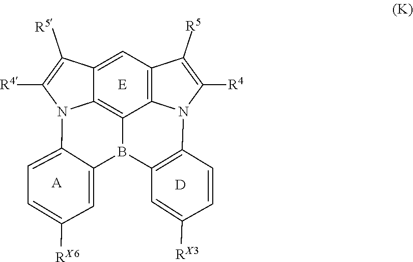

Preferred compounds (K) are mentioned in the following table, wherein Groups 1, 2 and 4 are defined as follows:

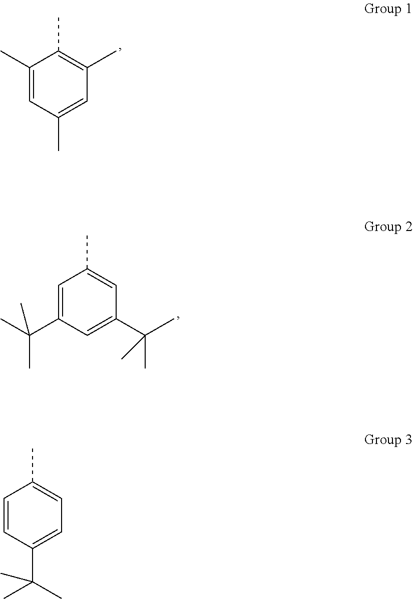

| R⁴, R^{4′} | R⁵, R^{5′} | R^{X3}, R^{X6} |
|---|---|---|
| Ph | Ph | tBu |
| Me | Group1 | tBu |
| Me | Group2 | tBu |
| Group2 | Group2 | tBu |
| isoPr | Group1 | tBu |
| Ph | Ph | H |
| Me | Group1 | H |
| Me | Group2 | H |
| Group2 | Group2 | H |
| isoPr | Group1 | H |

The compounds of formula

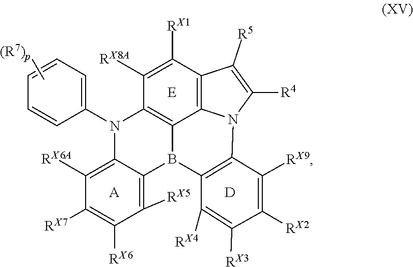

### Class 4

In the compounds of class 4 (formula (XV)), the groups, residues and indices R⁴, R⁵, R⁷, R^{X1}, R^{X2}, R^{X3}, R^{X4}, R^{X5}, R^{X6}, R^{X7}, R^{X9}, R^{X6A}, R^{X8A} and p are defined above.

In the case that R^{X6A} and R^{X7}, R^{X6} and R^{X7}, R^{X5} and R^{X6}, R^{X1} and R^{X8A}, R^{X2} and R^{X3} or R^{X3} and R^{X4} or R^{X2} and R^{X9} or two adjacent residues R⁷, in the compounds of class 4 may form together an unsubstituted or substituted ring, the following rings (a) and (b) are formed:

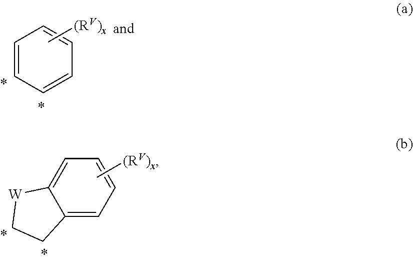

wherein

R^{V} represents H, a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group;

x represents 0, 1, 2, 3 or 4, preferably 0, 1 or 2, more preferably 0; and

W represents CR′″₂, O, S or NR^{IV};

R′″ represents a C₁-C₂₅ alkyl group, preferably a C₁-C₈ alkyl group, more preferably a C₁-C₄ alkyl group;

R^{IV} represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group; and

each * represents a point of attachment to ring A, D or E, or to the phenyl ring bearing the R⁷-substituent(s).

Most preferably, R⁴ and R⁵ in the compounds of class 4 each independently represents methyl, ethyl, iso-propyl, sec-propyl, n-butyl, —C(Me)₂C₂H₅, unsubstituted phenyl, p-tert-butyl-phenyl, mesityl, xylyl, O-methyl-phenyl or unsubstituted or substituted biphenyl, preferably unsubstituted biphenyl; or

R⁴ and R⁵ may form together an unsubstituted or substituted cyclohexene ring, preferably an unsubstituted cyclohexene ring.

An example for compounds of class 4, wherein R⁴ and R⁵ may form together an unsubstituted cyclohexene ring is the following compound:

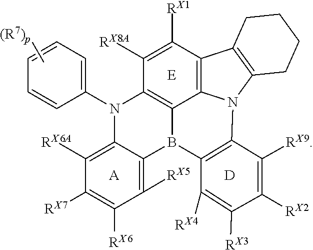

Most preferably, R^{X2}, R^{X3}, R^{X4} and R^{X9} in the compounds of class 4 each independently represents H, methyl, ethyl, iso-propyl, sec-propyl, n-butyl, tert-butyl, —C(Me)₂C₂H₅, unsubstituted phenyl, p-tert-butyl-phenyl, mesityl, xylyl, O-methyl-phenyl, unsubstituted or substituted biphenyl, preferably unsubstituted biphenyl or 2,4-difluoro(2,4-difluorophenyl).

Most preferably, R^{X4} and R^{X5} in the compounds of class 4 are H.

Further most preferably, R^{X4}, R^{X5} and R^{X9} in the compounds of class 4 are H

Most preferably, R^{X6}, R^{X6A}, R^{X7} and R⁷ in the compounds of class 4 each independently represents H, methyl, ethyl, iso-propyl, sec-propyl, n-butyl, tert-butyl, —C(Me)₂C₂H₅, unsubstituted phenyl, p-tert-butyl-phenyl, mesityl, xylyl, O-methyl-phenyl, unsubstituted or substituted biphenyl, preferably unsubstituted biphenyl or 2,4-difluoro(2,4-difluorophenyl); or R^{X6A} and one of the residues R⁷ in ortho position to the nitrogen atom, may form together a ring, wherein the ring is formed via a single bond, via a C₁-C₃ alkyl group which is optionally substituted by a C₁-C₂₅ alkyl group, preferably by a C₁-C₈ alkyl group, more preferably by a C₁-C₄ alkyl group, via an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group, via an unsubstituted or substituted C₂ alkenyl group, via a group NR′, via O, via a group POOR′ or via a unsubstituted or substituted P—C₆-C₃₀ aryl group, preferably unsubstituted or substituted P—C₆-C₁₀ aryl group, more preferably unsubstituted or substituted P—C₆ aryl group, most preferably unsubstituted P—C₆ aryl group; preferably via a single bond, wherein

R′ represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group.

Examples for compounds of class 4, wherein R^{X6A} and one of the residues R⁷ in ortho position to the nitrogen atom, form together a ring are the following compounds:

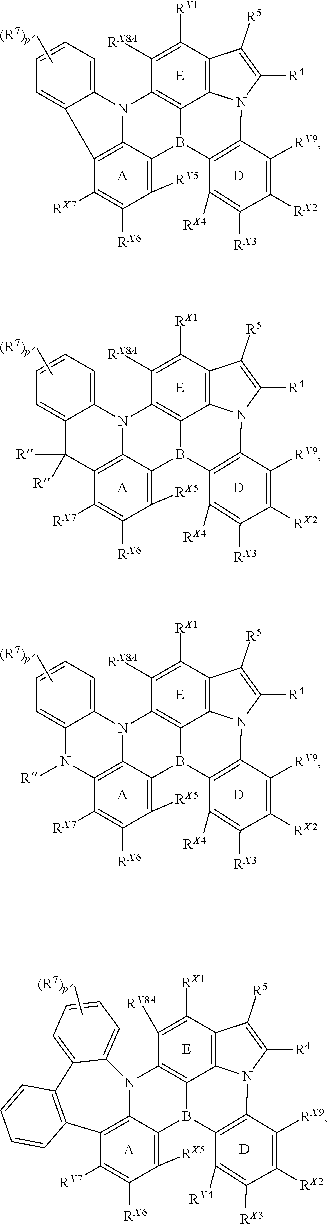

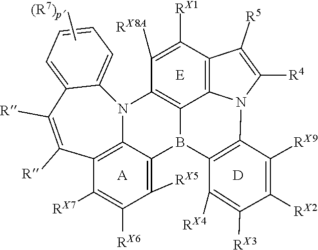

wherein

p′ is 0, 1, 2, 3 or 4, preferably 0, 1 or 2, more preferably 0 or 1;

R″ each independently represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group; and the other groups, residues and indices are as defined in formula (XV).

Examples for compounds of class 4 are compounds of the following formula (L), wherein the residues R⁴, R⁵, R⁷, R^{X3}, R^{X6} and R^{X7} are defined above.

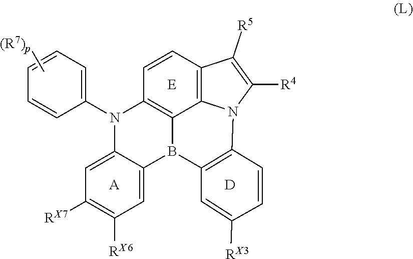

Preferred compounds (L) are mentioned in the following table, wherein Groups 1, 2, 4, 5, 6, 7 and 8 are defined as follows:

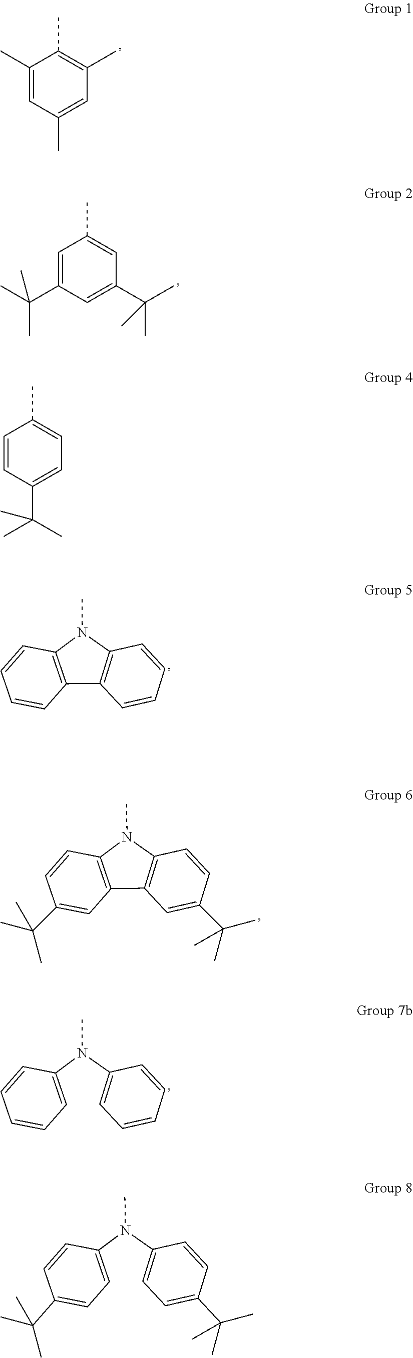

| R⁴ | R⁵ | R^{X6} | R^{X7} | R^{X3} | R⁷ |
|---|---|---|---|---|---|
| Ph | Ph | tBu | H | tBu | Group4 |
| Group4 | Group4 | tBu | H | tBu | Group4 |
| Me | Group1 | tBu | H | tBu | Group4 |
| Et | Group1 | tBu | H | tBu | Group4 |
| Ph | Ph | tBu | H | tBu | Group2 |
| Group4 | Group4 | tBu | H | tBu | Group2 |
| Me | Group1 | tBu | H | tBu | Group2 |
| Et | Group1 | tBu | H | tBu | Group2 |
| Ph | Ph | tBu | H | tBu | Group1 |
| Group4 | Group4 | tBu | H | tBu | Group1 |
| Group4 | Group4 | tBu | H | tBu | Group1 |
| Me | Group1 | tBu | H | tBu | Group1 |

Examples for compounds of class 4 are compounds of the following formula (M), wherein the residues R⁴, R⁵, R¹, R^{X3}, R^{X6} and R^{X7} are defined above.

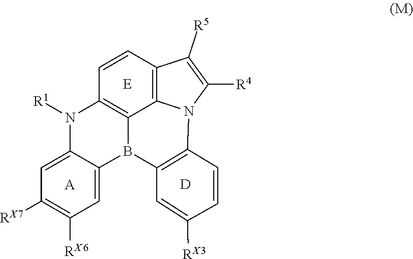

Preferred compounds (M) are mentioned in the following table, wherein Groups 1, 2, 4, 5, 6, 7, 8 and 10 are defined as follows:

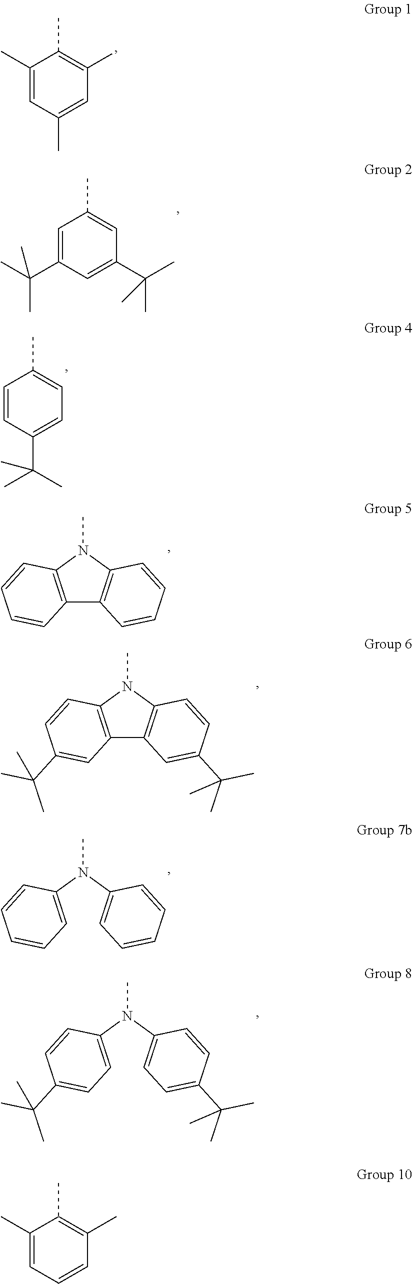

| R⁴ | R⁵ | R^{X6} | R^{X7} | R^{X3} | R¹ |
|---|---|---|---|---|---|
| Ph | Ph | H | Group5 | tBu | Group4 |
| Group4 | Group4 | H | Group5 | tBu | Group4 |
| Me | Group1 | H | Group5 | tBu | Group4 |
| Et | Group1 | H | Group5 | tBu | Group4 |
| Ph | Ph | H | Group6 | tBu | Group4 |
| Group4 | Group4 | H | Group6 | tBu | Group4 |
| Me | Group1 | H | Group6 | tBu | Group4 |
| Et | Group1 | H | Group6 | tBu | Group4 |
| Ph | Ph | H | Group7b | tBu | Group4 |
| Group4 | Group4 | H | Group7b | tBu | Group4 |
| Group4 | Group4 | H | Group7b | tBu | Group4 |
| Me | Group1 | H | Group7b | tBu | Group4 |
| Ph | Ph | H | Group8 | tBu | Group4 |
| Group4 | Group4 | H | Group8 | tBu | Group4 |
| Me | Group1 | H | Group8 | tBu | Group4 |
| Et | Group1 | H | Group8 | tBu | Group4 |
| Me | Group10 | tBu | H | tBu | Group4 |
| Et | Group10 | tBu | H | tBu | Group4 |
| Me | Group10 | tBu | H | tBu | Group2 |
| Et | Group10 | tBu | H | tBu | Group2 |
| Ph | Ph | tBu | H | tBu | Group10 |
| Group4 | Group4 | tBu | H | tBu | Group10 |
| Group4 | Group4 | tBu | H | tBu | Group10 |
| Me | Group10 | tBu | H | tBu | Group10 |
| Me | Group 10 | H | Group 5 | tBu | Group 4 |
| Et | Group 10 | H | Group 5 | tBu | Group 4 |
| Me | Group 10 | H | Group 6 | tBu | Group 4 |
| Et | Group 10 | H | Group 6 | tBu | Group 4 |
| Me | Group 10 | H | Group 7b | tBu | Group 4 |
| Me | Group 10 | H | Group 8 | tBu | Group 4 |
| Et | Group 10 | H | Group 8 | tBu | Group 4 |

The compounds of formula

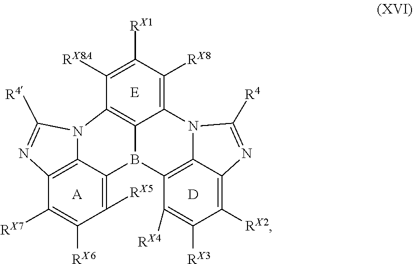

### Class 5

In the compounds of class 5 (formula (XVI)), the groups, residues and indices R⁴, R^{4′}, R^{X1}, R^{X2}, R^{X3}, R^{X4}, R^{X5}, R^{X6}, R^{X7}, R^{X8A} and R^{X8} are defined above.

In the case that R^{X6} and R^{X7}, R^{X5} and R^{X6}, R^{X1} and R^{X8A}, R^{X1} and R^{X8}, R^{X2} and R^{X3} and/or R^{X3} and R^{X4}, in the compounds of class 5 may form together an unsubstituted or substituted ring, the following rings (a) and (b) are formed:

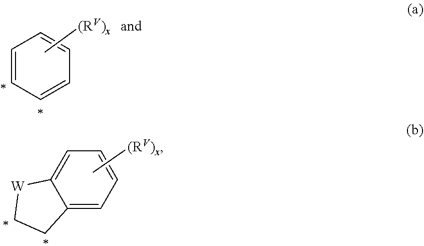

wherein

R^{V} represents H, a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group;

x represents 0, 1, 2, 3 or 4, preferably 0, 1 or 2, more preferably 0; and

W represents CR′″₂, O, S or NR^{IV};

R′″ represents a C₁-C₂₅ alkyl group, preferably a C₁-C₈ alkyl group, more preferably a C₁-C₄ alkyl group;

R^{IV} represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group; and

each * represents a point of attachment to ring A, D or E.

Most preferably, R⁴ and R^{4′} in the compounds of class 5 each independently represents methyl, tert-butyl, CF₃, unsubstituted phenyl, p-tert-butyl-phenyl, xylyl, or mesityl.

Most preferably, R^{X2}, R^{X3}, R^{X6} and R^{X7} in the compounds of class 5 each independently represents H, methyl, ethyl, iso-propyl, sec-propyl, n-butyl, tert-butyl, —C(Me)₂C₂H₅, unsubstituted phenyl, p-tert-butyl-phenyl, mesityl, xylyl, O-methyl-phenyl or unsubstituted or substituted biphenyl, preferably unsubstituted biphenyl.

Most preferably, R^{X1}, R^{X8} and R^{X8A} in the compounds of class 5 each independently represents H, methyl, ethyl, n-butyl, unsubstituted phenyl, —O-phenyl, —NPh₂, —N-carbazolyl or —N(CH₅^{t}Bu)₂; or

Most preferably, R^{X4} and R^{X5} in the compounds of class 5 are H, and further most preferably R^{X4}, R^{X5}, R^{X8} and R^{X8A} are H.

Examples for compounds of class 5 are compounds of the following formula (N), wherein the residues R⁴, R^{4′}, R^{X1}, R^{X2}, R^{X3}, R^{X6} and R^{X7} are defined above.

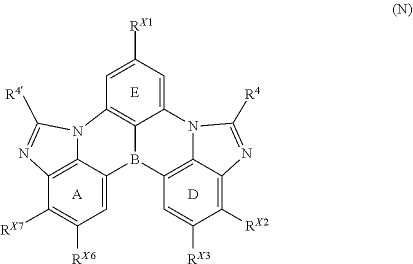

Preferred compounds (N) are mentioned in the following table, wherein Groups 1, 4 and 10 are defined as follows:

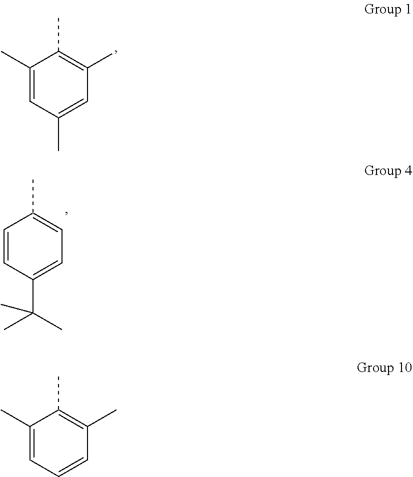

| R⁴ | R^{4′} | R^{X7} | R^{X6} | R^{X1} | R^{X3} | R^{X2} |
|---|---|---|---|---|---|---|
| Ph | Ph | H | H | H | H | H |
| tBu | tBu | H | H | H | H | H |
| Group 1 | Group1 | H | H | H | H | H |
| Group 4 | Group4 | H | H | H | H | H |
| CF3 | CF3 | H | H | H | H | H |
| Ph | Ph | H | H | Me | H | H |
| tBu | tBu | H | H | Me | H | H |
| Group 1 | Group1 | H | H | Me | H | H |
| Group 4 | Group4 | H | H | Me | H | H |
| CF3 | CF3 | H | H | Me | H | H |
| Ph | Ph | tBu | H | H | H | tBu |
| tBu | tBu | tBu | H | H | H | tBu |
| Group 1 | Group1 | tBu | H | H | H | tBu |
| Group 4 | Group4 | tBu | H | H | H | tBu |
| CF3 | CF3 | tBu | H | H | H | tBu |
| Ph | Ph | H | tBu | H | tBu | H |
| tBu | tBu | H | tBu | H | tBu | H |
| Group1 | Group1 | H | tBu | H | tBu | H |
| Group4 | Group4 | H | tBu | H | tBu | H |
| CF3 | CF3 | H | tBu | H | tBu | H |
| Ph | Ph | H | tBu | Me | tBu | H |
| tBu | tBu | H | tBu | Me | tBu | H |
| Group1 | Group1 | H | tBu | Me | tBu | H |
| Group4 | Group4 | H | tBu | Me | tBu | H |
| CF3 | CF3 | H | tBu | Me | tBu | H |
| Group10 | Group10 | H | H | H | H | H |
| Group10 | Group10 | H | H | Me | H | H |
| Group10 | Group10 | tBu | H | H | H | tBu |
| Group10 | Group10 | H | tBu | H | tBu | H |
| Group10 | Group10 | H | tBu | Me | tBu | H |
| Group10 | Group10 | Group10 | H | H | H | Group10 |
| Group1 | Group1 | Group1 | H | H | H | Group1 |
| Group10 | Group10 | Group1 | H | H | H | Group1 |
| Group1 | Group1 | Group10 | H | H | H | Group10 |
| Group10 | Group10 | Group10 | H | Me | H | Group10 |
| Group1 | Group1 | Group1 | H | Me | H | Group1 |
| Group10 | Group10 | Group1 | H | Me | H | Group1 |
| Group1 | Group1 | Group10 | H | Me | H | Group10 |
| Group10 | Group10 | Ph | H | H | H | Ph |
| Group1 | Group1 | Ph | H | H | H | Ph |
| Group10 | Group10 | Ph | H | Me | H | Ph |
| Group1 | Group1 | Ph | H | Me | H | Ph |

Examples for further compounds of formula

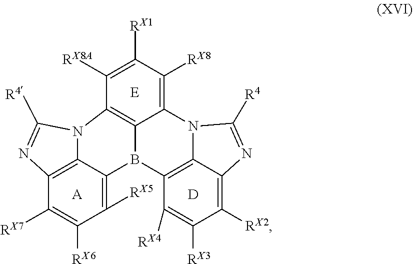

are the following compounds:

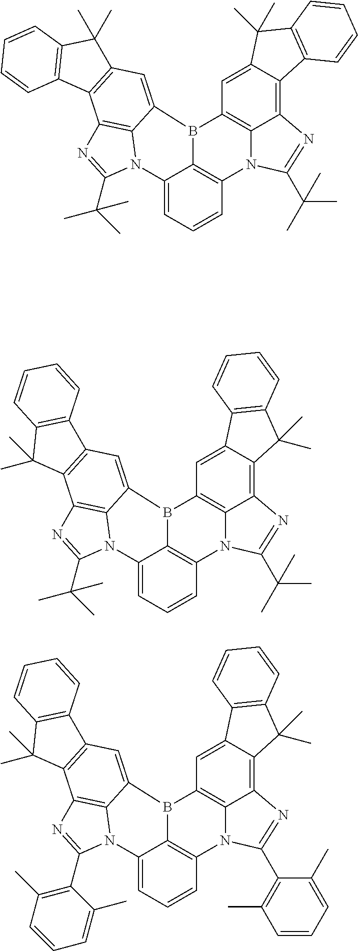

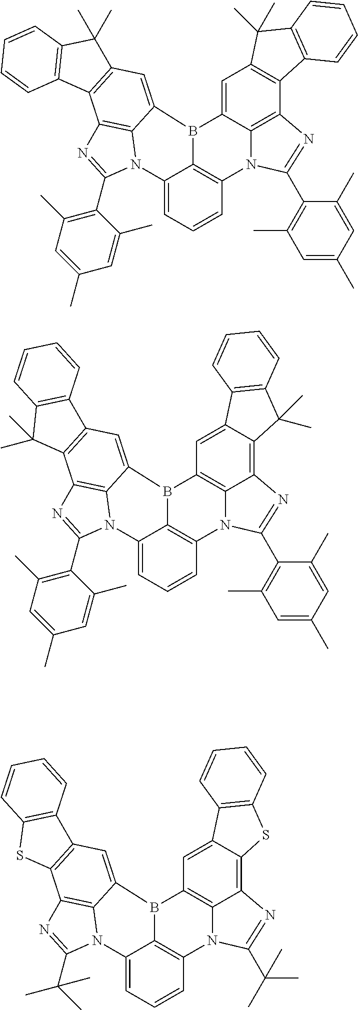

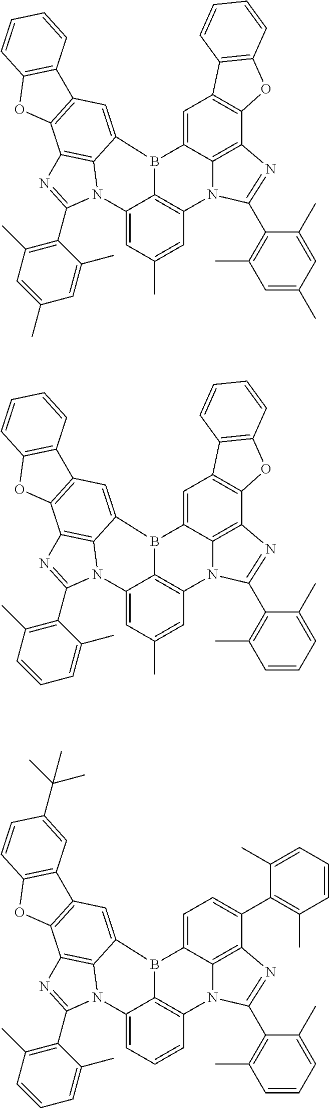 [SMILES: Cc1cc(C)c(-c2nc3c4oc5ccccc5c4cc4c3n2-c2cc(C)cc3c2B4c2cc4c5ccccc5oc4c4nc(-c5c(C)cc(C)cc5C)n-3c24)c(C)c1 ; Cc1cc2c3c(c1)-n1c(-c4c(C)cccc4C)nc4c5oc6ccccc6c5cc(c41)B3c1cc3c4ccccc4oc3c3nc(-c4c(C)cccc4C)n-2c13 ; Cc1cccc(C)c1-c1ccc2c3c1nc(-c1c(C)cccc1C)n3-c1cccc3c1B2c1cc2c4cc(C(C)(C)C)ccc4oc2c2nc(-c4c(C)cccc4C)n-3c12]

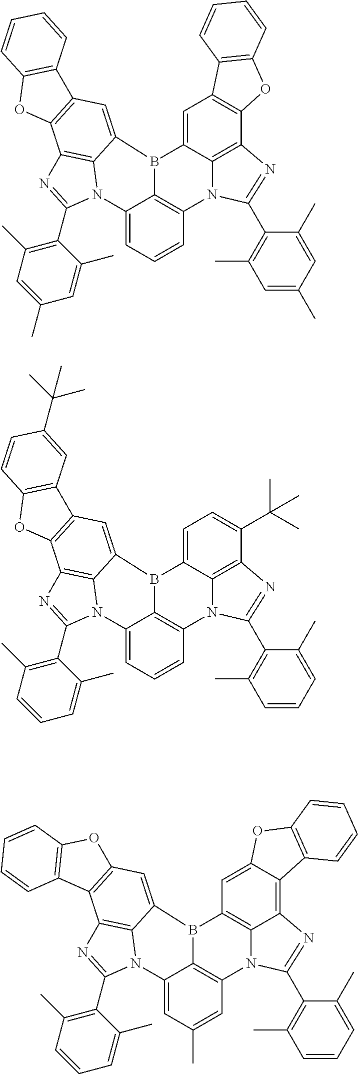

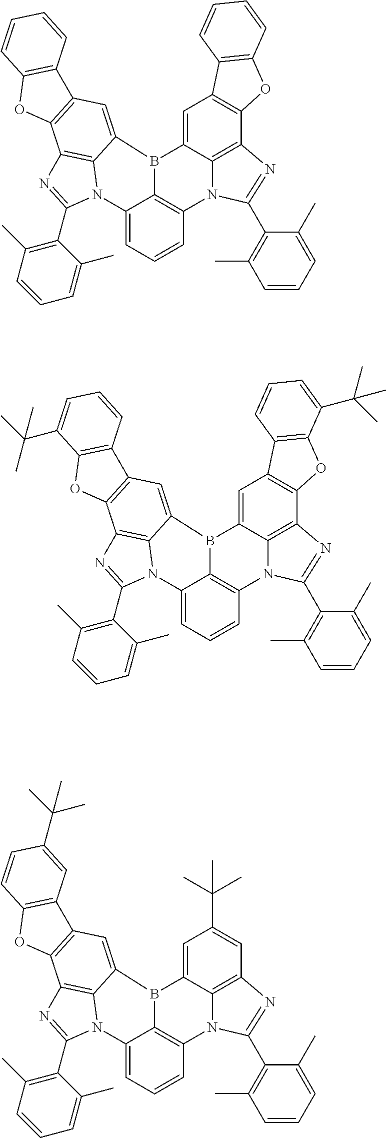

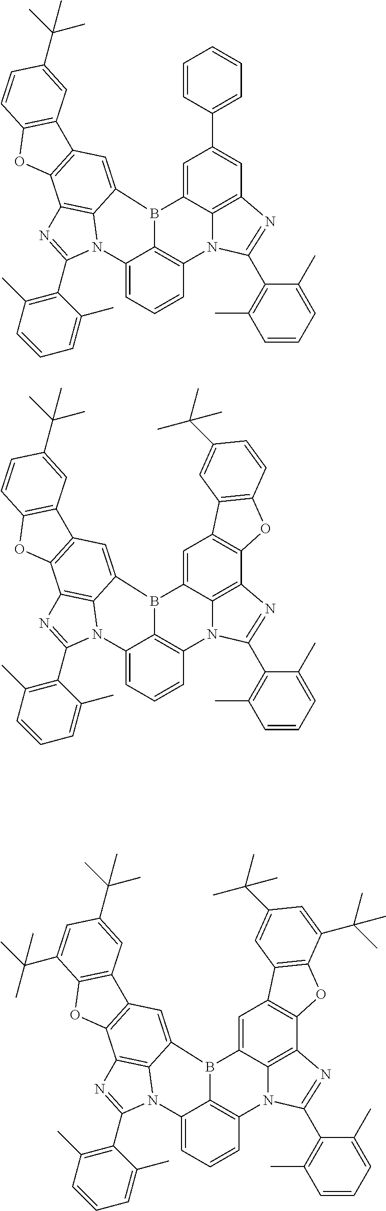

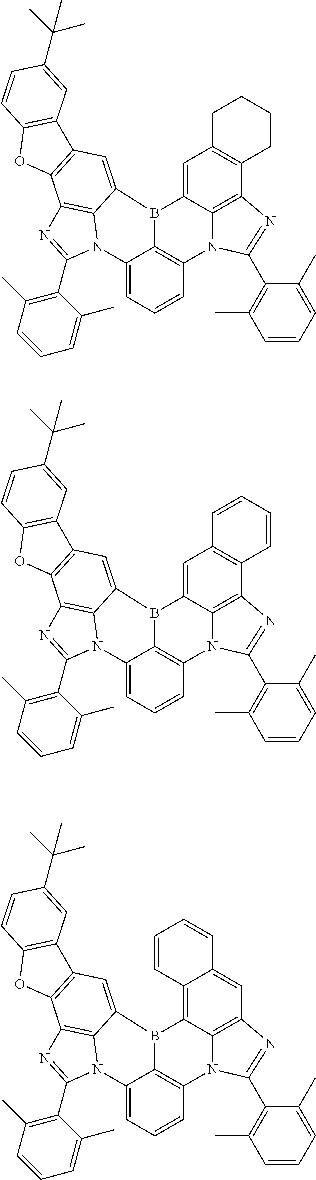 [SMILES: Cc1cccc(C)c1-c1nc2c3c(cc4c2n1-c1cccc2c1B4c1cc4c5cc(C(C)(C)C)ccc5oc4c4nc(-c5c(C)cccc5C)n-2c14)CCCC3 ; Cc1cccc(C)c1-c1nc2c3ccccc3cc3c2n1-c1cccc2c1B3c1cc3c4cc(C(C)(C)C)ccc4oc3c3nc(-c4c(C)cccc4C)n-2c13 ; Cc1cccc(C)c1-c1nc2cc3ccccc3c3c2n1-c1cccc2c1B3c1cc3c4cc(C(C)(C)C)ccc4oc3c3nc(-c4c(C)cccc4C)n-2c13]

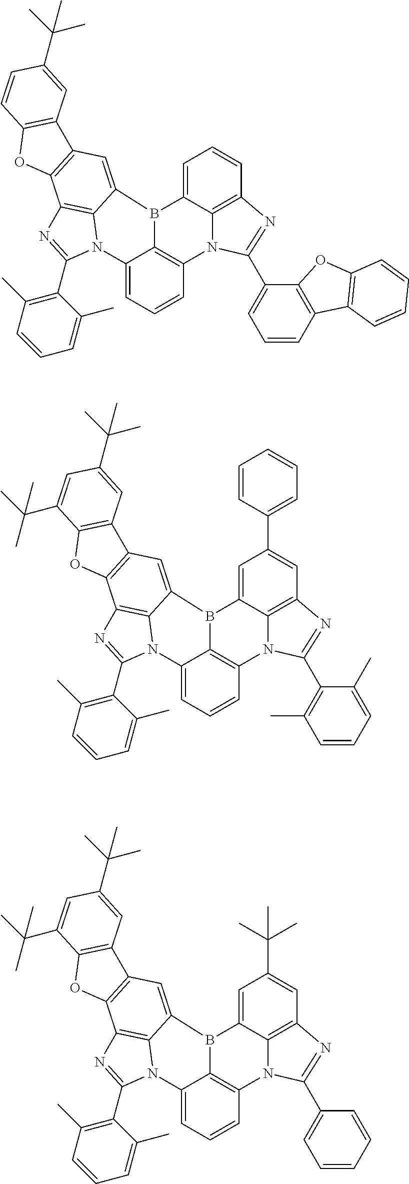

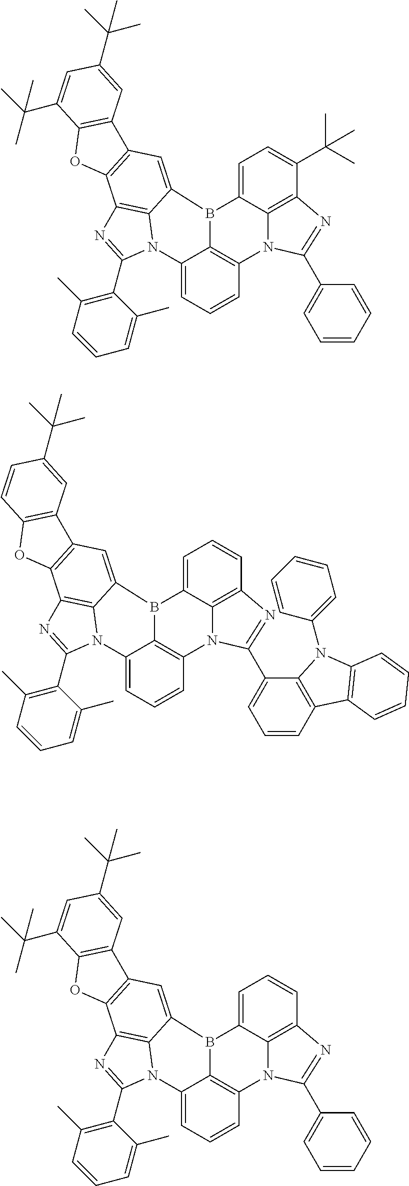 [SMILES: Cc1cccc(C)c1-c1nc2c3oc4c(C(C)(C)C)cc(C(C)(C)C)cc4c3cc3c2n1-c1cccc2c1B3c1ccc(C(C)(C)C)c3nc(-c4ccccc4)n-2c13 ; Cc1cccc(C)c1-c1nc2c3oc4ccc(C(C)(C)C)cc4c3cc3c2n1-c1cccc2c1B3c1cccc3nc(-c4cccc5c6ccccc6n(-c6ccccc6)c45)n-2c13 ; Cc1cccc(C)c1-c1nc2c3oc4c(C(C)(C)C)cc(C(C)(C)C)cc4c3cc3c2n1-c1cccc2c1B3c1cccc3nc(-c4ccccc4)n-2c13]

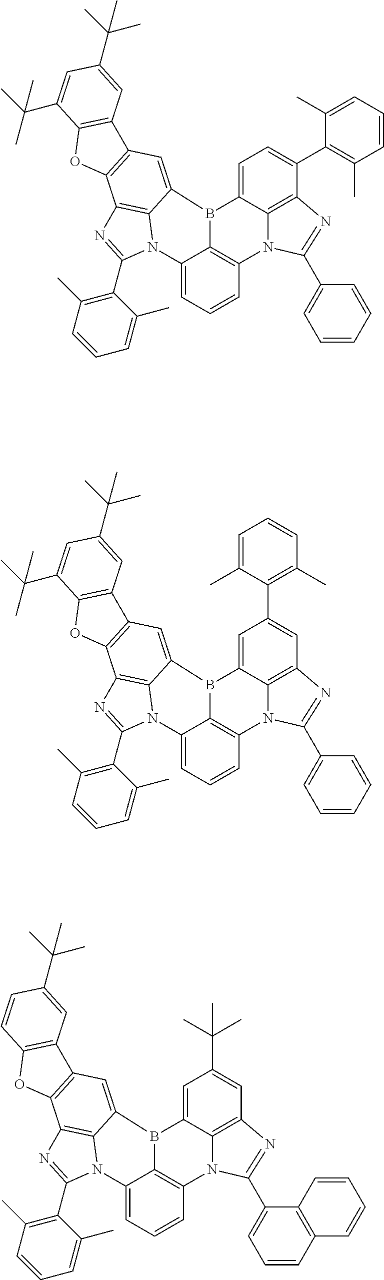 [SMILES: Cc1cccc(C)c1-c1ccc2c3c1nc(-c1ccccc1)n3-c1cccc3c1B2c1cc2c4cc(C(C)(C)C)cc(C(C)(C)C)c4oc2c2nc(-c4c(C)cccc4C)n-3c12 ; Cc1cccc(C)c1-c1cc2c3c(c1)nc(-c1ccccc1)n3-c1cccc3c1B2c1cc2c4cc(C(C)(C)C)cc(C(C)(C)C)c4oc2c2nc(-c4c(C)cccc4C)n-3c12 ; Cc1cccc(C)c1-c1nc2c3oc4ccc(C(C)(C)C)cc4c3cc3c2n1-c1cccc2c1B3c1cc(C(C)(C)C)cc3nc(-c4cccc5ccccc45)n-2c13]

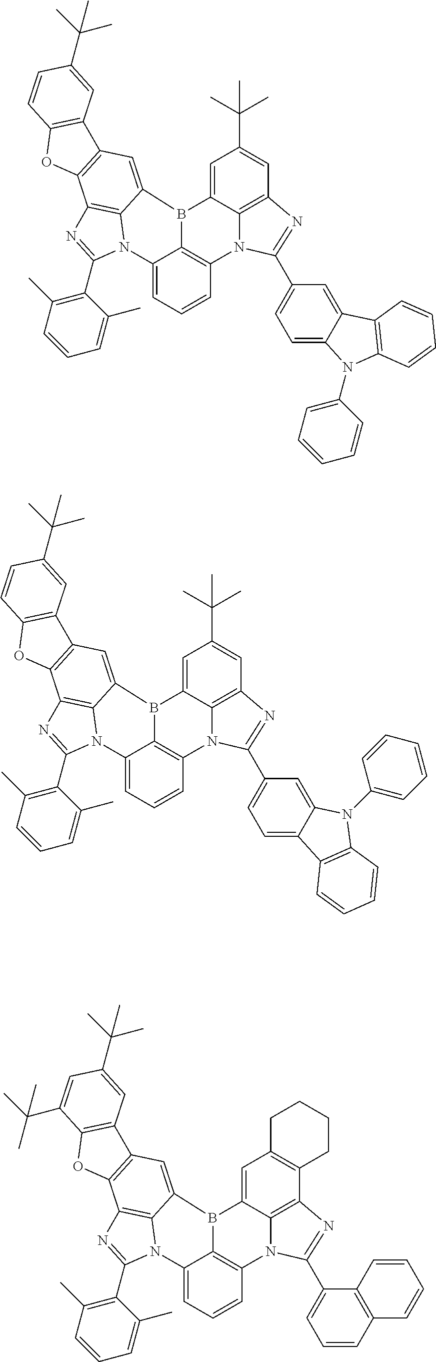 [SMILES: Cc1cccc(C)c1-c1nc2c3oc4ccc(C(C)(C)C)cc4c3cc3c2n1-c1cccc2c1B3c1cc(C(C)(C)C)cc3nc(-c4ccc5c(c4)c4ccccc4n5-c4ccccc4)n-2c13 ; Cc1cccc(C)c1-c1nc2c3oc4ccc(C(C)(C)C)cc4c3cc3c2n1-c1cccc2c1B3c1cc(C(C)(C)C)cc3nc(-c4ccc5c6ccccc6n(-c6ccccc6)c5c4)n-2c13 ; Cc1cccc(C)c1-c1nc2c3oc4c(C(C)(C)C)cc(C(C)(C)C)cc4c3cc3c2n1-c1cccc2c1B3c1cc3c(c4nc(-c5cccc6ccccc56)n-2c14)CCCC3]

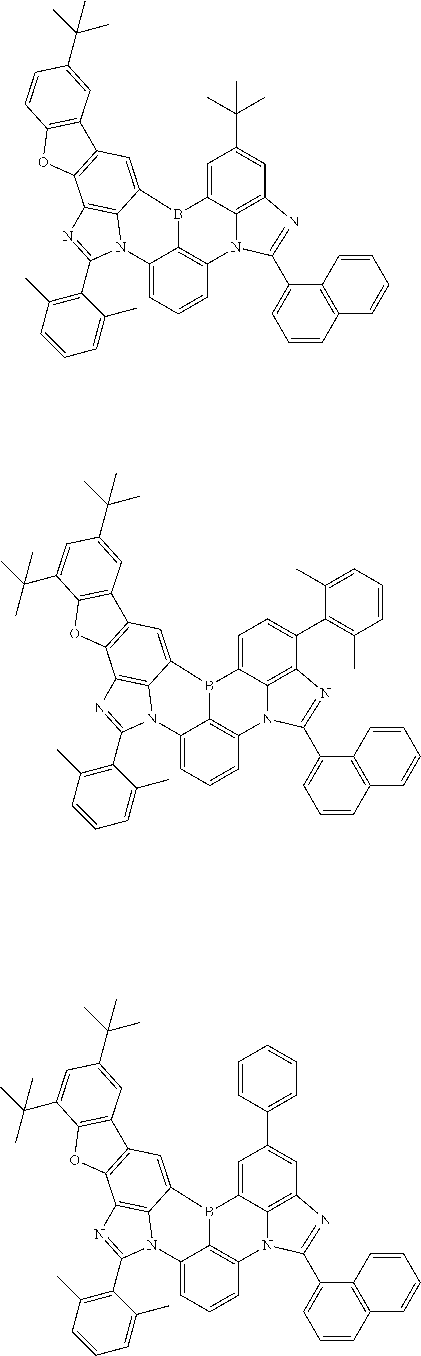 [SMILES: Cc1cccc(C)c1-c1nc2c3oc4ccc(C(C)(C)C)cc4c3cc3c2n1-c1cccc2c1B3c1cc(C(C)(C)C)cc3nc(-c4cccc5ccccc45)n-2c13 ; Cc1cccc(C)c1-c1ccc2c3c1nc(-c1cccc4ccccc14)n3-c1cccc3c1B2c1cc2c4cc(C(C)(C)C)cc(C(C)(C)C)c4oc2c2nc(-c4c(C)cccc4C)n-3c12 ; Cc1cccc(C)c1-c1nc2c3oc4c(C(C)(C)C)cc(C(C)(C)C)cc4c3cc3c2n1-c1cccc2c1B3c1cc(-c3ccccc3)cc3nc(-c4cccc5ccccc45)n-2c13]

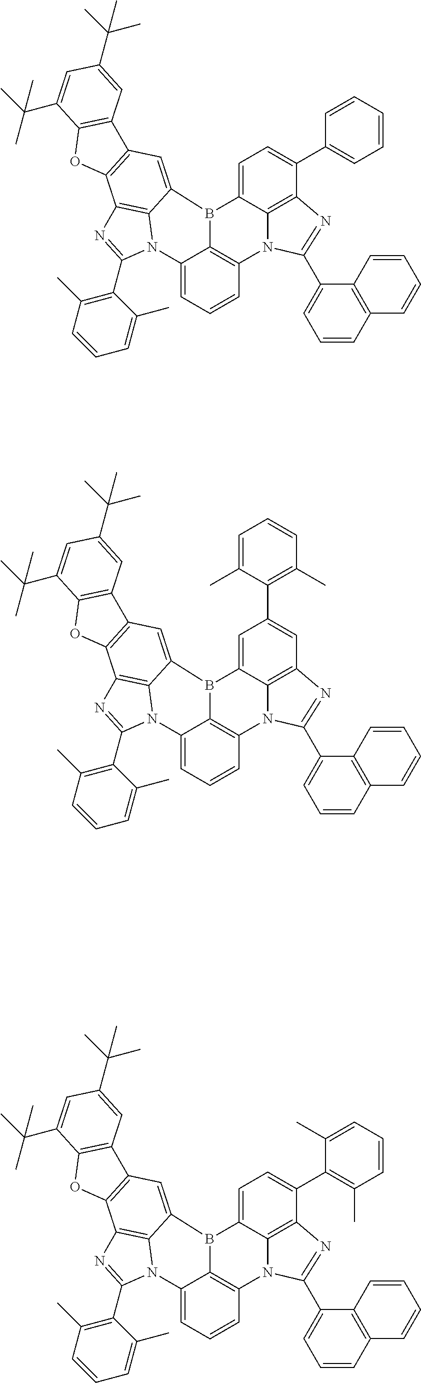 [SMILES: Cc1cccc(C)c1-c1nc2c3oc4c(C(C)(C)C)cc(C(C)(C)C)cc4c3cc3c2n1-c1cccc2c1B3c1ccc(-c3ccccc3)c3nc(-c4cccc5ccccc45)n-2c13 ; Cc1cccc(C)c1-c1cc2c3c(c1)nc(-c1cccc4ccccc14)n3-c1cccc3c1B2c1cc2c4cc(C(C)(C)C)cc(C(C)(C)C)c4oc2c2nc(-c4c(C)cccc4C)n-3c12 ; Cc1cccc(C)c1-c1ccc2c3c1nc(-c1cccc4ccccc14)n3-c1cccc3c1B2c1cc2c4cc(C(C)(C)C)cc(C(C)(C)C)c4oc2c2nc(-c4c(C)cccc4C)n-3c12]

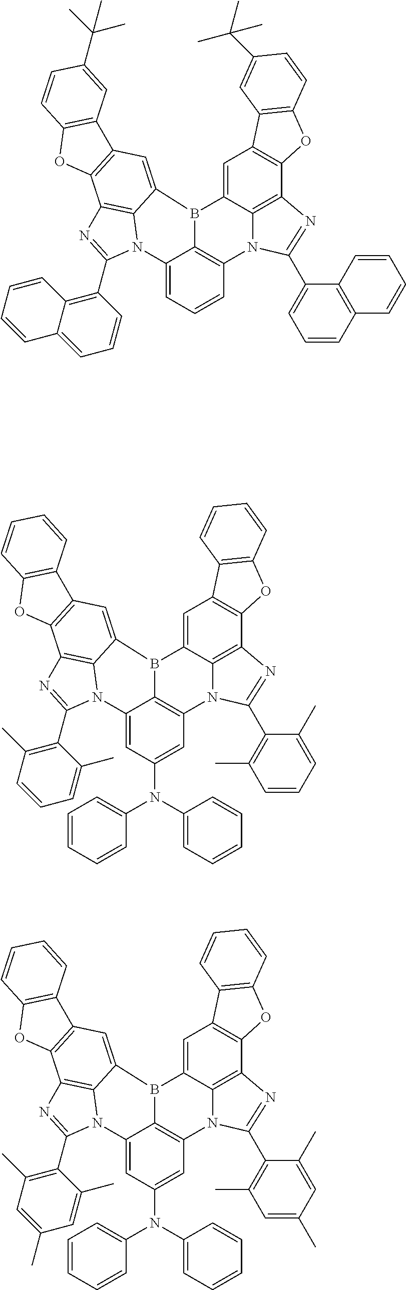 [SMILES: CC(C)(C)c1ccc2oc3c(cc4c5c3nc(-c3cccc6ccccc36)n5-c3cccc5c3B4c3cc4c6cc(C(C)(C)C)ccc6oc4c4nc(-c6cccc7ccccc67)n-5c34)c2c1 ; Cc1cccc(C)c1-c1nc2c3oc4ccccc4c3cc3c2n1-c1cc(N(c2ccccc2)c2ccccc2)cc2c1B3c1cc3c4ccccc4oc3c3nc(-c4c(C)cccc4C)n-2c13 ; Cc1cc(C)c(-c2nc3c4oc5ccccc5c4cc4c3n2-c2cc(N(c3ccccc3)c3ccccc3)cc3c2B4c2cc4c5ccccc5oc4c4nc(-c5c(C)cc(C)cc5C)n-3c24)c(C)c1]

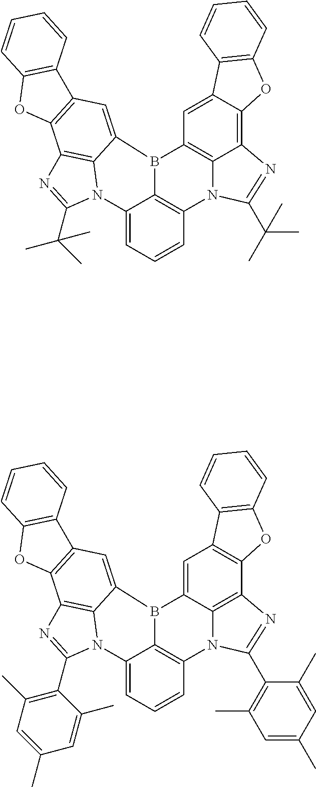 [SMILES: CC(C)(C)c1nc2c3oc4ccccc4c3cc3c2n1-c1cccc2c1B3c1cc3c4ccccc4oc3c3nc(C(C)(C)C)n-2c13 ; Cc1cc(C)c(-c2nc3c4oc5ccccc5c4cc4c3n2-c2cccc3c2B4c2cc4c5ccccc5oc4c4nc(-c5c(C)cc(C)cc5C)n-3c24)c(C)c1]

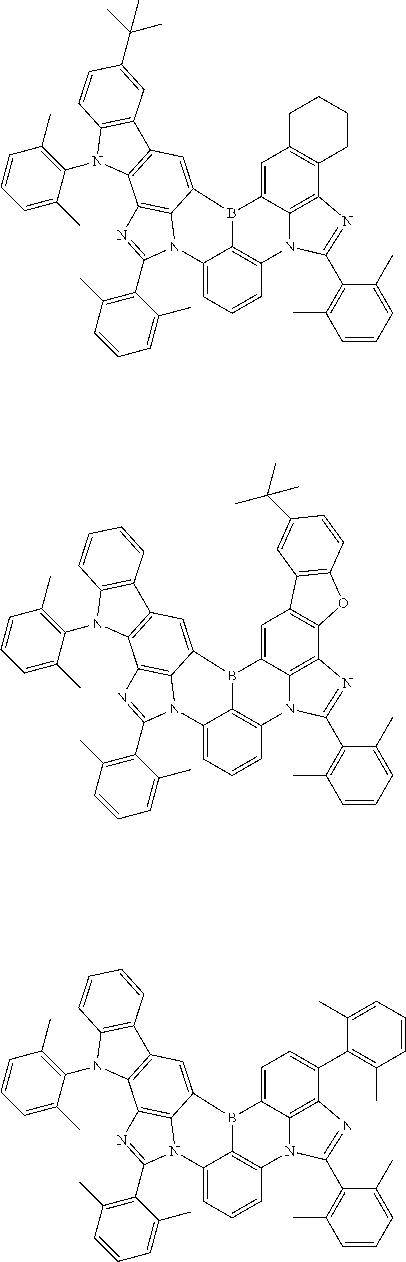 [SMILES: Cc1cccc(C)c1-c1nc2c3c(cc4c2n1-c1cccc2c1B4c1cc4c5cc(C(C)(C)C)ccc5n(-c5c(C)cccc5C)c4c4nc(-c5c(C)cccc5C)n-2c14)CCCC3 ; Cc1cccc(C)c1-c1nc2c3oc4ccc(C(C)(C)C)cc4c3cc3c2n1-c1cccc2c1B3c1cc3c4ccccc4n(-c4c(C)cccc4C)c3c3nc(-c4c(C)cccc4C)n-2c13 ; Cc1cccc(C)c1-c1ccc2c3c1nc(-c1c(C)cccc1C)n3-c1cccc3c1B2c1cc2c4ccccc4n(-c4c(C)cccc4C)c2c2nc(-c4c(C)cccc4C)n-3c12]

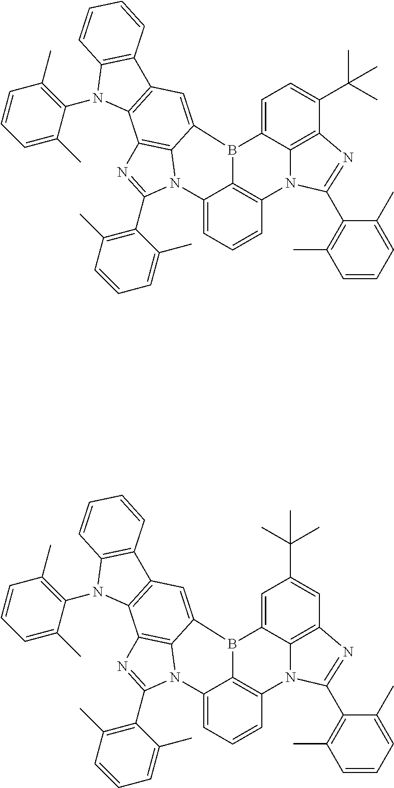 [SMILES: Cc1cccc(C)c1-c1nc2c(C(C)(C)C)ccc3c2n1-c1cccc2c1B3c1cc3c4ccccc4n(-c4c(C)cccc4C)c3c3nc(-c4c(C)cccc4C)n-2c13 ; Cc1cccc(C)c1-c1nc2cc(C(C)(C)C)cc3c2n1-c1cccc2c1B3c1cc3c4ccccc4n(-c4c(C)cccc4C)c3c3nc(-c4c(C)cccc4C)n-2c13]

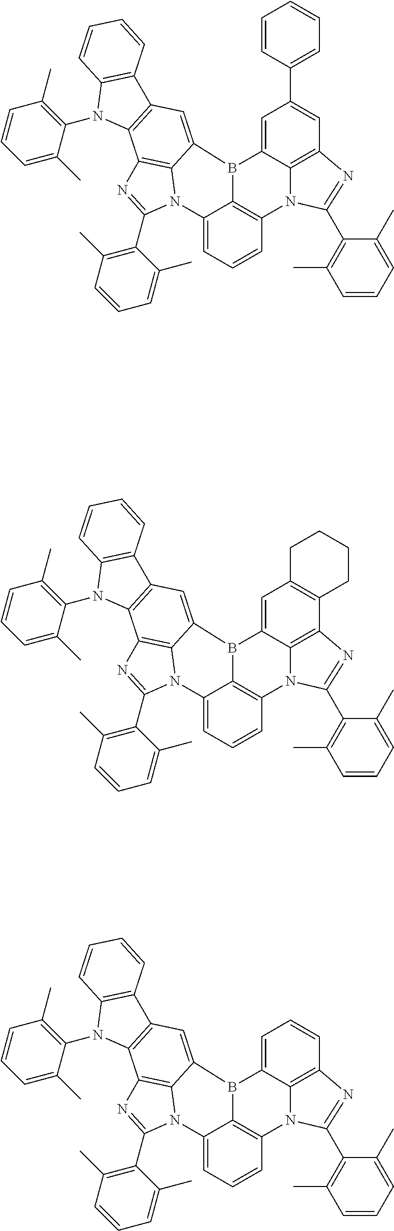 [SMILES: Cc1cccc(C)c1-c1nc2cc(-c3ccccc3)cc3c2n1-c1cccc2c1B3c1cc3c4ccccc4n(-c4c(C)cccc4C)c3c3nc(-c4c(C)cccc4C)n-2c13 ; Cc1cccc(C)c1-c1nc2c3c(cc4c2n1-c1cccc2c1B4c1cc4c5ccccc5n(-c5c(C)cccc5C)c4c4nc(-c5c(C)cccc5C)n-2c14)CCCC3 ; Cc1cccc(C)c1-c1nc2cccc3c2n1-c1cccc2c1B3c1cc3c4ccccc4n(-c4c(C)cccc4C)c3c3nc(-c4c(C)cccc4C)n-2c13]

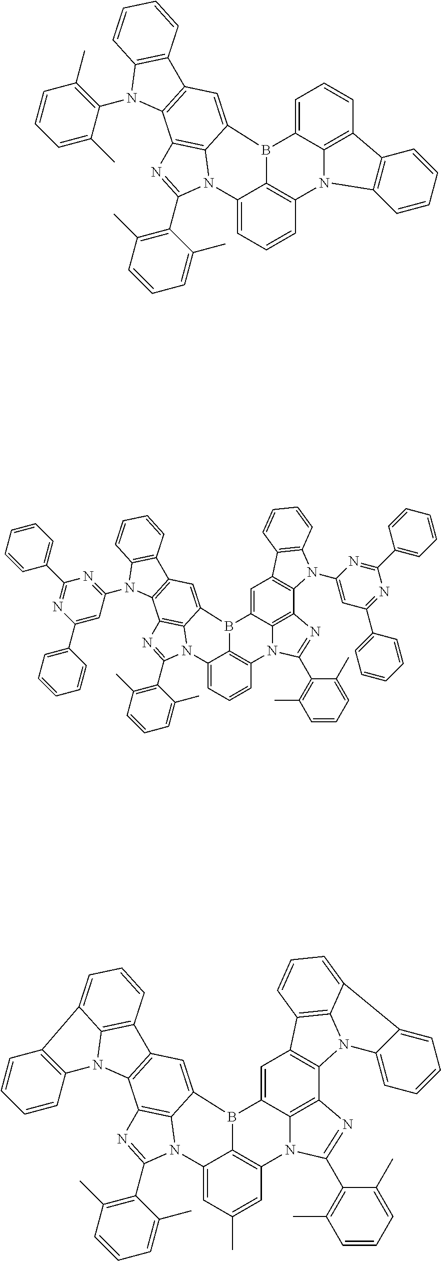 [SMILES: Cc1cccc(C)c1-c1nc2c3c(cc4c5ccccc5n(-c5c(C)cccc5C)c42)B2c4c(cccc4-n4c5ccccc5c5cccc2c54)-n13 ; Cc1cccc(C)c1-c1nc2c3c(cc4c5ccccc5n(-c5cc(-c6ccccc6)nc(-c6ccccc6)n5)c42)B2c4c(cccc4-n4c(-c5c(C)cccc5C)nc5c4c2cc2c4ccccc4n(-c4cc(-c6ccccc6)nc(-c6ccccc6)n4)c25)-n13 ; Cc1cc2c3c(c1)-n1c(-c4c(C)cccc4C)nc4c1c(cc1c5cccc6c7ccccc7n(c65)c14)B3c1cc3c4cccc5c6ccccc6n(c54)c3c3nc(-c4c(C)cccc4C)n-2c13]

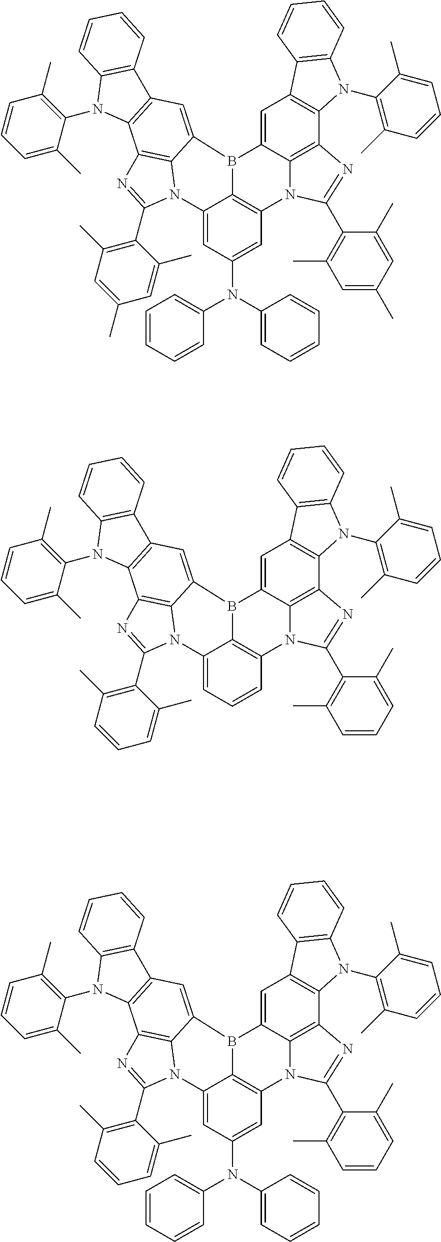 [SMILES: Cc1cc(C)c(-c2nc3c4c(cc5c6ccccc6n(-c6c(C)cccc6C)c53)B3c5c(cc(N(c6ccccc6)c6ccccc6)cc5-n5c(-c6c(C)cc(C)cc6C)nc6c5c3cc3c5ccccc5n(-c5c(C)cccc5C)c36)-n24)c(C)c1 ; Cc1cccc(C)c1-c1nc2c3c(cc4c5ccccc5n(-c5c(C)cccc5C)c42)B2c4c(cccc4-n4c(-c5c(C)cccc5C)nc5c4c2cc2c4ccccc4n(-c4c(C)cccc4C)c25)-n13 ; Cc1cccc(C)c1-c1nc2c3c(cc4c5ccccc5n(-c5c(C)cccc5C)c42)B2c4c(cc(N(c5ccccc5)c5ccccc5)cc4-n4c(-c5c(C)cccc5C)nc5c4c2cc2c4ccccc4n(-c4c(C)cccc4C)c25)-n13]

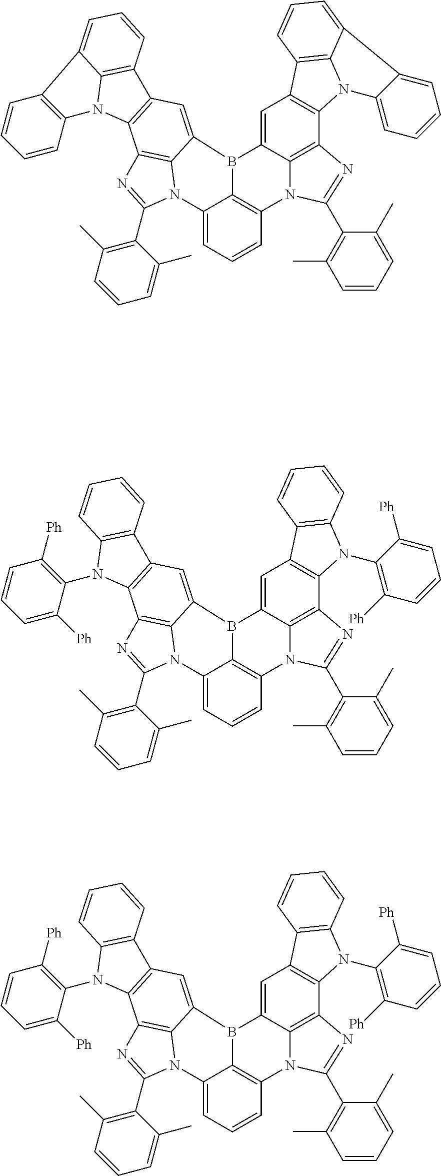 [SMILES: Cc1cccc(C)c1-c1nc2c3c(cc4c5cccc6c7ccccc7n(c65)c42)B2c4c(cccc4-n4c(-c5c(C)cccc5C)nc5c4c2cc2c4cccc6c7ccccc7n(c64)c25)-n13 ; Cc1cccc(C)c1-c1nc2c3c(cc4c5ccccc5n(-c5c(-c6ccccc6)cccc5-c5ccccc5)c42)B2c4c(cccc4-n4c(-c5c(C)cccc5C)nc5c4c2cc2c4ccccc4n(-c4c(-c6ccccc6)cccc4-c4ccccc4)c25)-n13 ; Cc1cccc(C)c1-c1nc2c3c(cc4c5ccccc5n(-c5c(-c6ccccc6)cccc5-c5ccccc5)c42)B2c4c(cccc4-n4c(-c5c(C)cccc5C)nc5c4c2cc2c4ccccc4n(-c4c(-c6ccccc6)cccc4-c4ccccc4)c25)-n13]

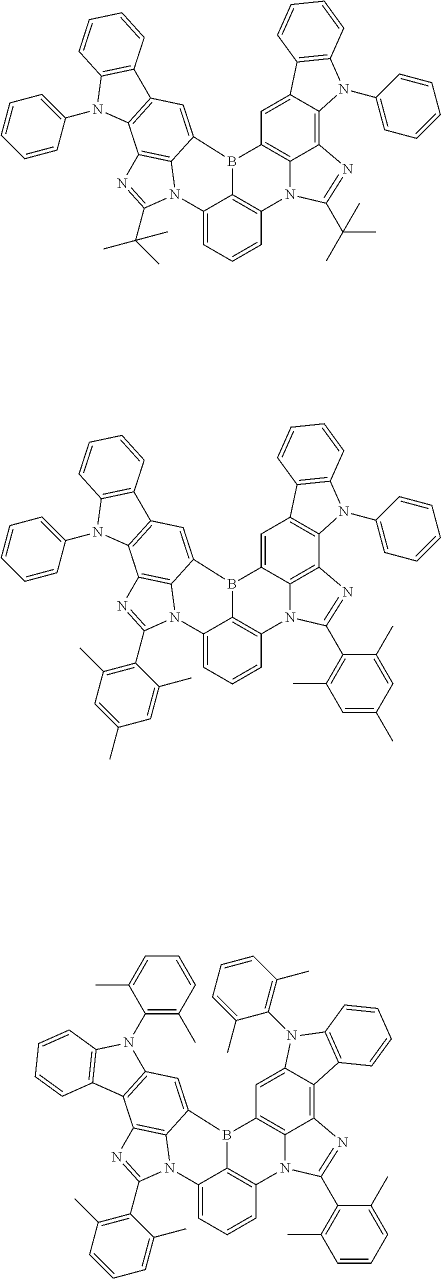 [SMILES: CC(C)(C)c1nc2c3c(cc4c5ccccc5n(-c5ccccc5)c42)B2c4c(cccc4-n4c(C(C)(C)C)nc5c4c2cc2c4ccccc4n(-c4ccccc4)c25)-n13 ; Cc1cc(C)c(-c2nc3c4c(cc5c6ccccc6n(-c6ccccc6)c53)B3c5c(cccc5-n5c(-c6c(C)cc(C)cc6C)nc6c5c3cc3c5ccccc5n(-c5ccccc5)c36)-n24)c(C)c1 ; Cc1cccc(C)c1-c1nc2c3c4ccccc4n(-c4c(C)cccc4C)c3cc3c2n1-c1cccc2c1B3c1cc3c(c4ccccc4n3-c3c(C)cccc3C)c3nc(-c4c(C)cccc4C)n-2c13]

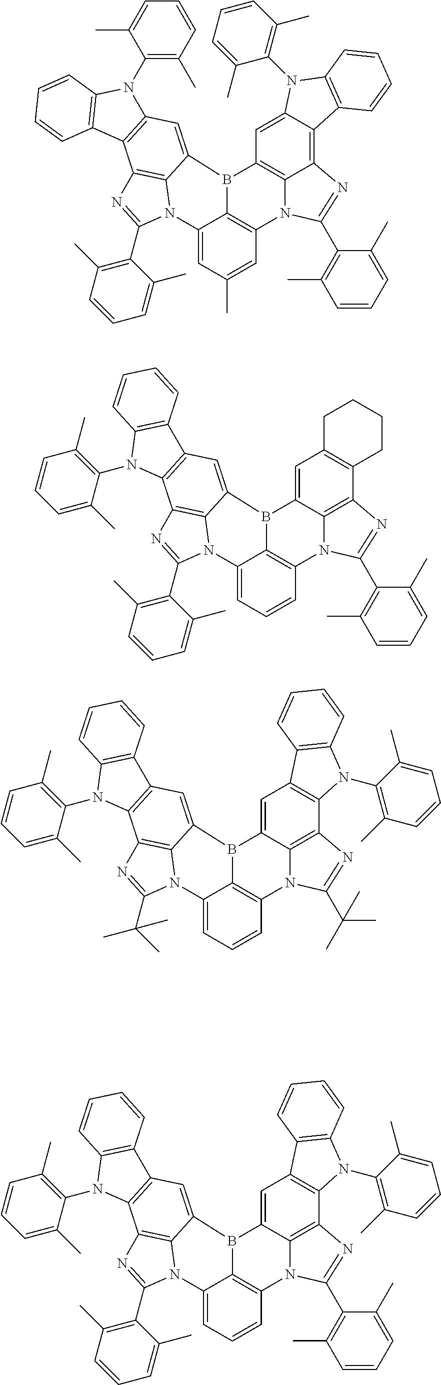 [SMILES: Cc1cc2c3c(c1)-n1c(-c4c(C)cccc4C)nc4c5c6ccccc6n(-c6c(C)cccc6C)c5cc(c41)B3c1cc3c(c4ccccc4n3-c3c(C)cccc3C)c3nc(-c4c(C)cccc4C)n-2c13 ; Cc1cccc(C)c1-c1nc2c3c(cc4c2n1-c1cccc2c1B4c1cc4c5ccccc5n(-c5c(C)cccc5C)c4c4nc(-c5c(C)cccc5C)n-2c14)CCCC3 ; Cc1cccc(C)c1-n1c2ccccc2c2cc3c4c(nc(C(C)(C)C)n4-c4cccc5c4B3c3cc4c6ccccc6n(-c6c(C)cccc6C)c4c4nc(C(C)(C)C)n-5c34)c21 ; Cc1cccc(C)c1-c1nc2c3c(cc4c5ccccc5n(-c5c(C)cccc5C)c42)B2c4c(cccc4-n4c(-c5c(C)cccc5C)nc5c4c2cc2c4ccccc4n(-c4c(C)cccc4C)c25)-n13]

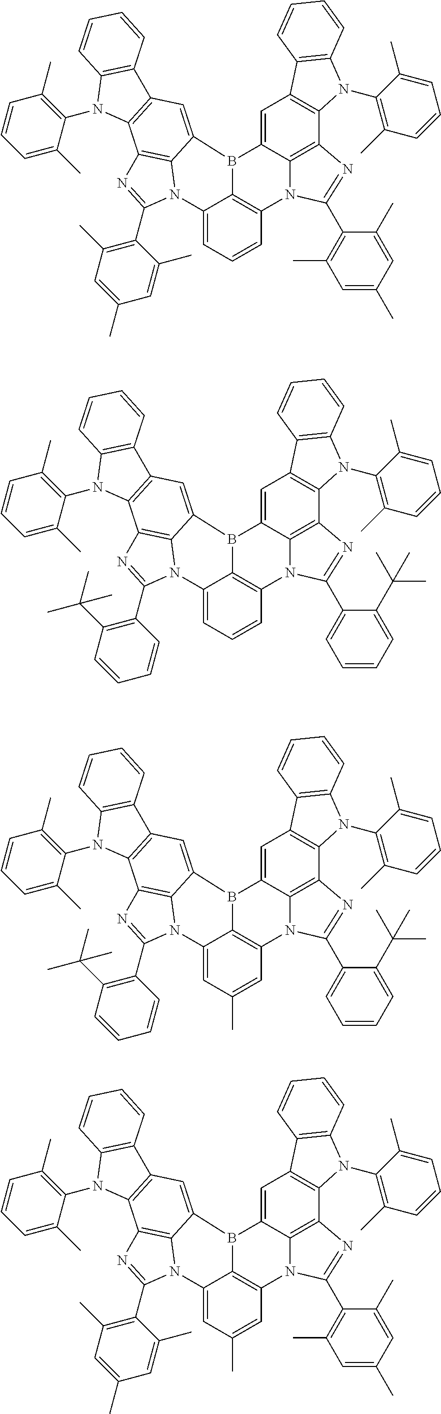

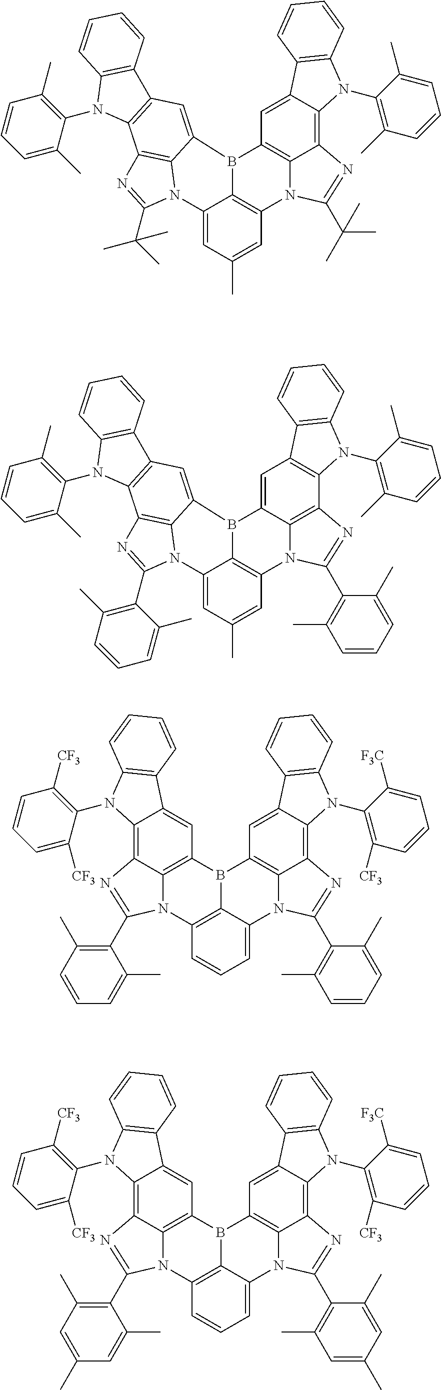

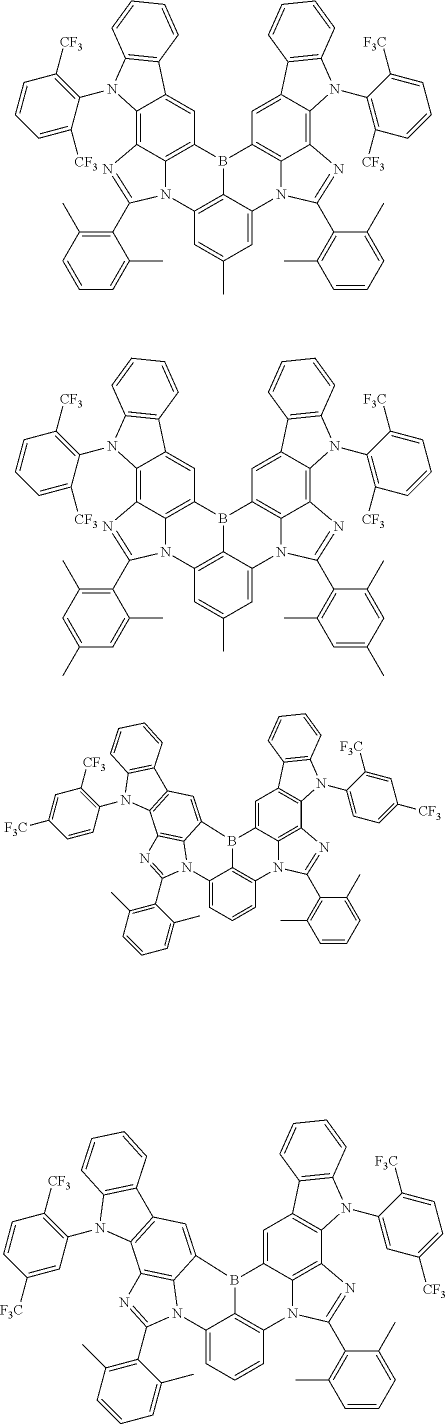

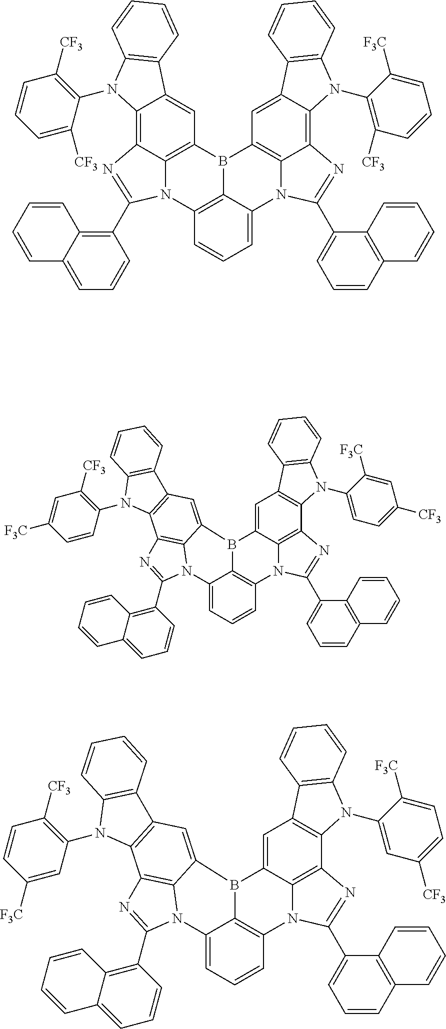 [SMILES: FC(F)(F)c1cccc(C(F)(F)F)c1-n1c2ccccc2c2cc3c4c(nc(-c5cccc6ccccc56)n4-c4cccc5c4B3c3cc4c6ccccc6n(-c6c(C(F)(F)F)cccc6C(F)(F)F)c4c4nc(-c6cccc7ccccc67)n-5c34)c21 ; FC(F)(F)c1ccc(-n2c3ccccc3c3cc4c5c(nc(-c6cccc7ccccc67)n5-c5cccc6c5B4c4cc5c7ccccc7n(-c7ccc(C(F)(F)F)cc7C(F)(F)F)c5c5nc(-c7cccc8ccccc78)n-6c45)c32)c(C(F)(F)F)c1 ; FC(F)(F)c1ccc(C(F)(F)F)c(-n2c3ccccc3c3cc4c5c(nc(-c6cccc7ccccc67)n5-c5cccc6c5B4c4cc5c7ccccc7n(-c7cc(C(F)(F)F)ccc7C(F)(F)F)c5c5nc(-c7cccc8ccccc78)n-6c45)c32)c1]

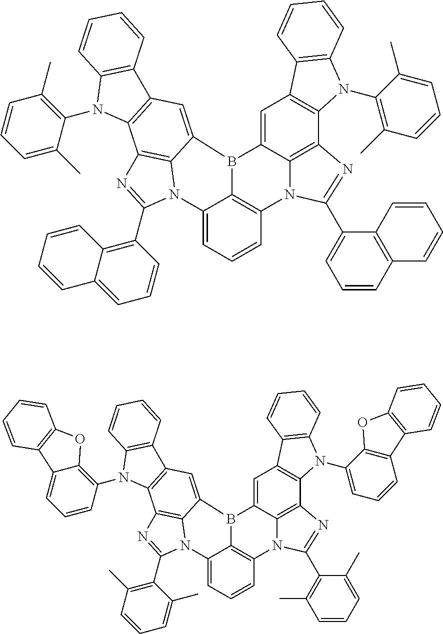 [SMILES: Cc1cccc(C)c1-n1c2ccccc2c2cc3c4c(nc(-c5cccc6ccccc56)n4-c4cccc5c4B3c3cc4c6ccccc6n(-c6c(C)cccc6C)c4c4nc(-c6cccc7ccccc67)n-5c34)c21 ; Cc1cccc(C)c1-c1nc2c3c(cc4c5ccccc5n(-c5cccc6c5oc5ccccc56)c42)B2c4c(cccc4-n4c(-c5c(C)cccc5C)nc5c4c2cc2c4ccccc4n(-c4cccc6c4oc4ccccc46)c25)-n13]

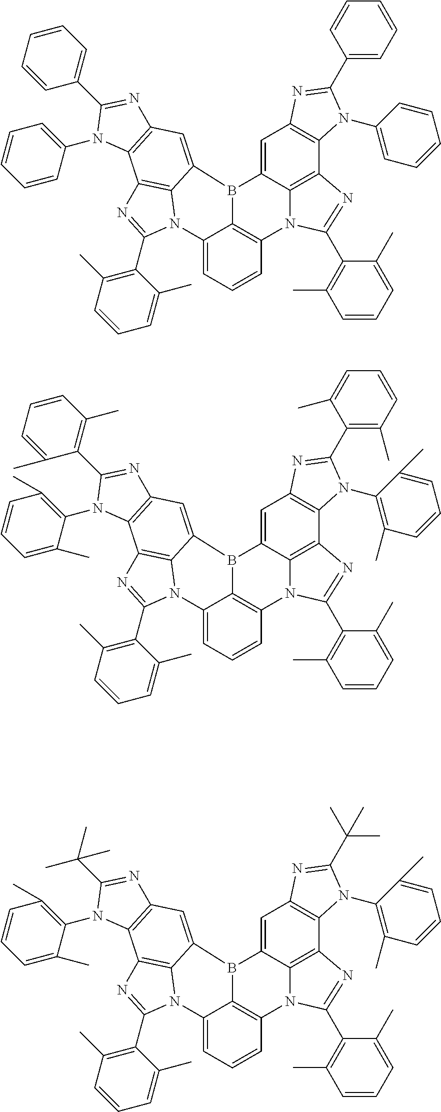 [SMILES: Cc1cccc(C)c1-c1nc2c3c(cc4nc(-c5ccccc5)n(-c5ccccc5)c42)B2c4c(cccc4-n4c(-c5c(C)cccc5C)nc5c4c2cc2nc(-c4ccccc4)n(-c4ccccc4)c25)-n13 ; Cc1cccc(C)c1-c1nc2c3c(cc4nc(-c5c(C)cccc5C)n(-c5c(C)cccc5C)c42)B2c4c(cccc4-n4c(-c5c(C)cccc5C)nc5c4c2cc2nc(-c4c(C)cccc4C)n(-c4c(C)cccc4C)c25)-n13 ; Cc1cccc(C)c1-c1nc2c3c(cc4nc(C(C)(C)C)n(-c5c(C)cccc5C)c42)B2c4c(cccc4-n4c(-c5c(C)cccc5C)nc5c4c2cc2nc(C(C)(C)C)n(-c4c(C)cccc4C)c25)-n13]

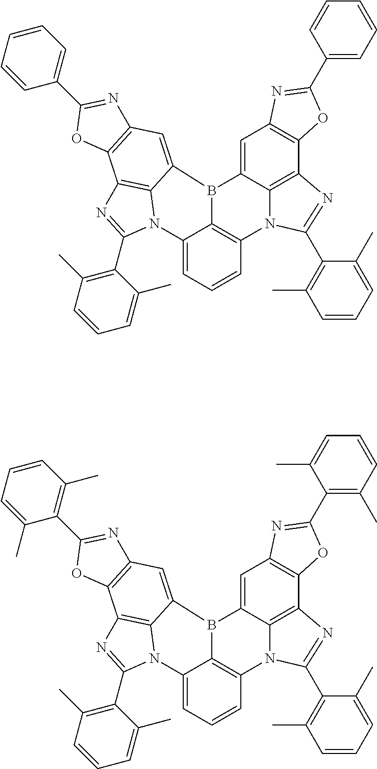 [SMILES: Cc1cccc(C)c1-c1nc2c3oc(-c4ccccc4)nc3cc3c2n1-c1cccc2c1B3c1cc3nc(-c4ccccc4)oc3c3nc(-c4c(C)cccc4C)n-2c13 ; Cc1cccc(C)c1-c1nc2cc3c4c(nc(-c5c(C)cccc5C)n4-c4cccc5c4B3c3cc4nc(-c6c(C)cccc6C)oc4c4nc(-c6c(C)cccc6C)n-5c34)c2o1]

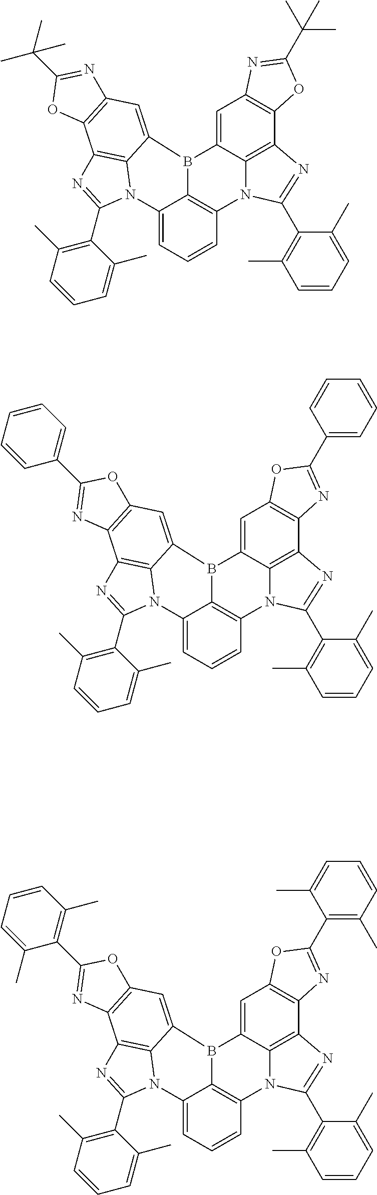 [SMILES: Cc1cccc(C)c1-c1nc2c3oc(C(C)(C)C)nc3cc3c2n1-c1cccc2c1B3c1cc3nc(C(C)(C)C)oc3c3nc(-c4c(C)cccc4C)n-2c13 ; Cc1cccc(C)c1-c1nc2c3nc(-c4ccccc4)oc3cc3c2n1-c1cccc2c1B3c1cc3oc(-c4ccccc4)nc3c3nc(-c4c(C)cccc4C)n-2c13 ; Cc1cccc(C)c1-c1nc2c(cc3c4c2nc(-c2c(C)cccc2C)n4-c2cccc4c2B3c2cc3oc(-c5c(C)cccc5C)nc3c3nc(-c5c(C)cccc5C)n-4c23)o1]

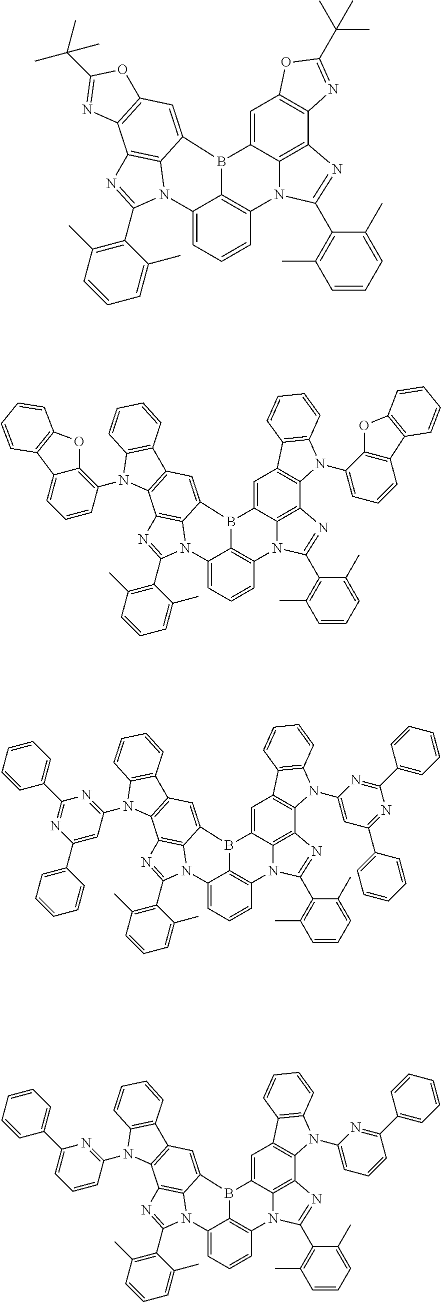

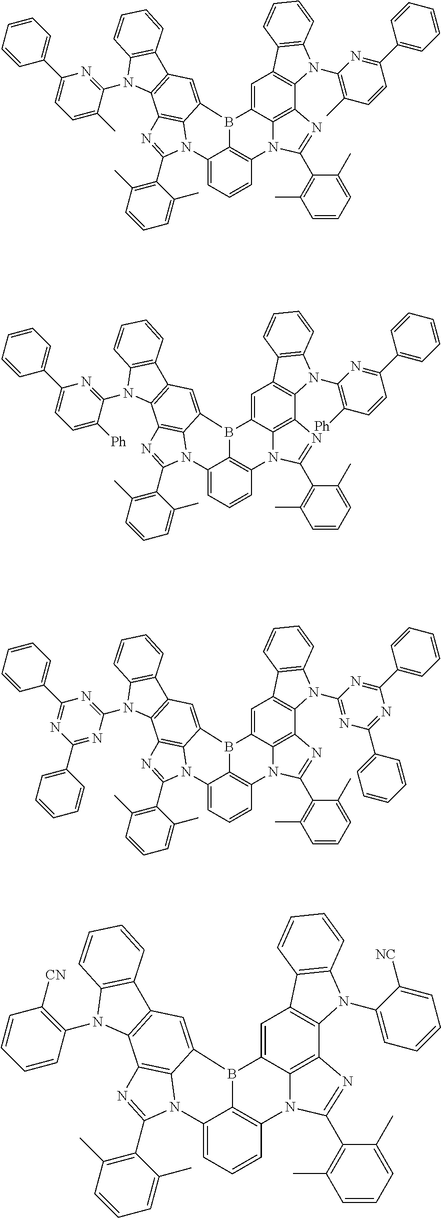

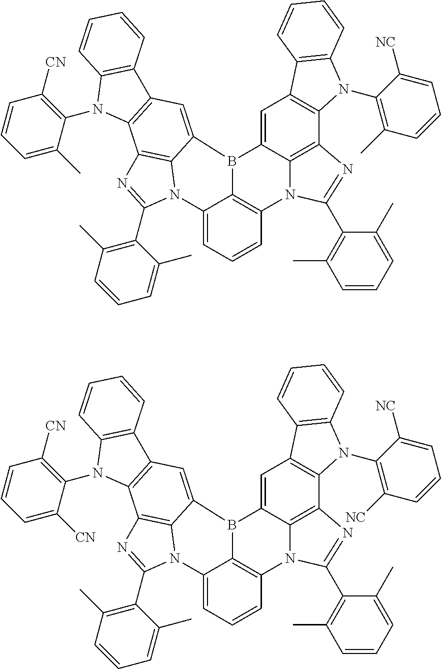 [SMILES: Cc1cccc(C)c1-c1nc2c3c(cc4c5ccccc5n(-c5c(C)cccc5C#N)c42)B2c4c(cccc4-n4c(-c5c(C)cccc5C)nc5c4c2cc2c4ccccc4n(-c4c(C)cccc4C#N)c25)-n13 ; Cc1cccc(C)c1-c1nc2c3c(cc4c5ccccc5n(-c5c(C#N)cccc5C#N)c42)B2c4c(cccc4-n4c(-c5c(C)cccc5C)nc5c4c2cc2c4ccccc4n(-c4c(C#N)cccc4C#N)c25)-n13]

The compounds of formula

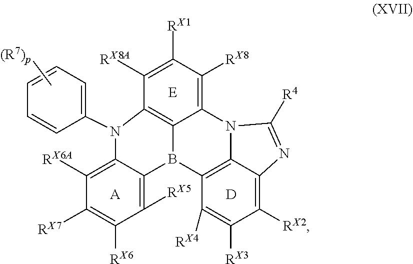

### Class 6

In the compounds of class 6 (formula (XVII)), the groups, residues and indices R⁴, R⁷, R^{X1}, R^{X2}, R^{X3}, R^{X4}, R^{X5}, R^{X6}, R^{X7}, R^{X8A}, R^{X6A}, R^{X8} and p are defined above.

In the case that R^{X6A} and R^{X7}, R^{X6} and R^{X7}, R^{X5} and R^{X6}, R^{X1} and R^{X8}, R^{X1} and R^{X8A}, R^{X2} and R^{X3}, R^{X3} and R^{X4} and/or two adjacent residues R⁷, in the compounds of class 6 may form together an unsubstituted or substituted ring, the following rings (a) and (b) are formed:

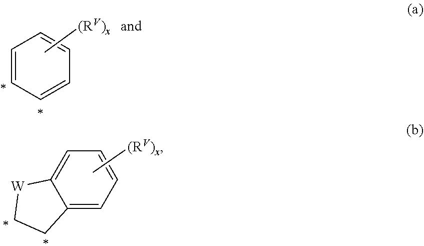

wherein

R^{V} represents H, a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group;

x represents 0, 1, 2, 3 or 4, preferably 0, 1 or 2, more preferably 0; and

W represents CR′″₂, O, S or NR^{IV};

R′″ represents a C₁-C₂₅ alkyl group, preferably a C₁-C₈ alkyl group, more preferably a C₁-C₄ alkyl group;

R^{IV} represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group; and

each * represents a point of attachment to ring A, D or E, or to the phenyl ring bearing the R⁷-substituent(s).

In the case that R⁴ is a substituted aryl group having 6 to 30 ring carbon atoms, preferably a substituted phenyl group, suitable substituents are a C₁-C₂₀ alkyl group, preferably a C₁-C₈ alkyl group, more preferably a C₁-C₄ alkyl group, or a unsubstituted or substituted C₆-C₃₀ aryl group, preferably a unsubstituted or substituted phenyl group; or

in the case that R⁴ in the compounds of class 6 is substituted phenyl, R⁴ may form a ring together with R^{X8}. An example for a compound of class 6, wherein R⁴ forms a ring together with R^{X8} is the following compound:

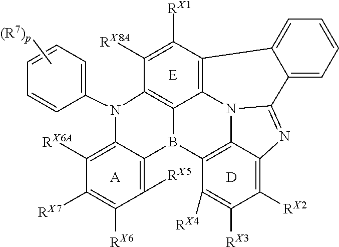

wherein the groups, residues and indices are as defined in formula (XVII).

Most preferably, R⁴ in the compounds of class 6 represents methyl, ethyl, iso-propyl, sec-propyl, n-butyl, —C(Me)₂C₂H₅, CF₃, unsubstituted phenyl, p-tert-butyl-phenyl, mesityl, xylyl, o-methyl-phenyl or unsubstituted or substituted biphenyl, preferably unsubstituted biphenyl.

Most preferably, R^{X2}, R^{X3} and R^{X4} and R^{X5} in the compounds of class 6 each independently represents H, methyl, ethyl, iso-propyl, sec-propyl, n-butyl, tert-butyl, —C(Me)₂C₂H₅, F, unsubstituted phenyl, p-tert-butyl-phenyl, mesityl, xylyl, O-methyl-phenyl, unsubstituted or substituted biphenyl, preferably unsubstituted biphenyl or 2,4-difluoro(2,4-difluorophenyl).

Most preferably, R^{X4} and R^{X5} in the compounds of class 6 are H.

Most preferably, R^{X6}, R^{X6A}, R^{X7} and R⁷ in the compounds of class 6 each independently represents H, methyl, ethyl, iso-propyl, sec-propyl, n-butyl, tert-butyl, —C(Me)₂C₂H₅, unsubstituted phenyl, p-tert-butyl-phenyl, mesityl, xylyl, O-methyl-phenyl, unsubstituted or substituted biphenyl, preferably unsubstituted biphenyl or 2,4-difluoro(2,4-difluorophenyl); or R^{X6A} and one of the residues R⁷ in ortho position to the nitrogen atom, may form together a ring, wherein the ring is formed via a single bond, via a C₁-C₃ alkyl group which is optionally substituted by a C₁-C₂₅ alkyl group, preferably by a C₁-C₈ alkyl group, more preferably by a C₁-C₄ alkyl group, via an unsubstituted or substituted C₆-C₃₀ aryl group, preferably unsubstituted or substituted C₆-C₁ aryl group, more preferably unsubstituted or substituted C₆ aryl group, most preferably unsubstituted C₆ aryl group, via an unsubstituted or substituted C₂ alkenyl group, via a group NR′, via O, via a group POOR′ or via a unsubstituted or substituted P—C₆-C₃₀ aryl group, preferably unsubstituted or substituted P—C₆-C₁₀ aryl group, more preferably unsubstituted or substituted P—C₆ aryl group, most preferably unsubstituted P—C₆ aryl group; preferably via a single bond, wherein

R′ represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group.

Examples for compounds of class 6, wherein R^{X6A} and one of the residues R⁷ in ortho position to the nitrogen atom, may form together a ring are the following compounds:

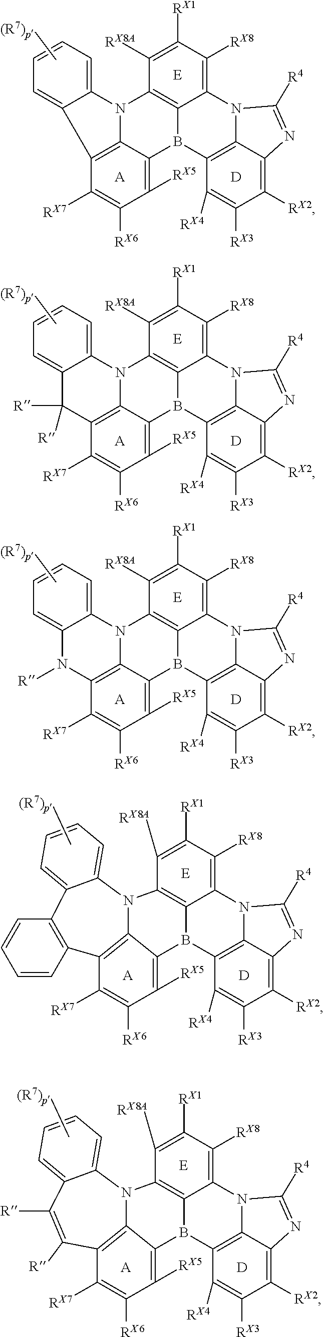

wherein

p′ is 0, 1, 2, 3 or 4, preferably 0, 1 or 2, more preferably 0 or 1;

R″ each independently represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C aryl group, most preferably unsubstituted C aryl group;

and the other groups, residues and indices are as defined in formula (XII).

Most preferably, R^{X1}, R^{X8} and R^{X8A} in the compounds of class 6 each independently represents H, methyl, ethyl, iso-propyl, sec-propyl, n-butyl, tert-butyl, —C(Me)₂C₂H₅, unsubstituted phenyl, p-tert-butyl-phenyl, mesityl, xylyl, O-methyl-phenyl, unsubstituted or substituted biphenyl, preferably unsubstituted biphenyl or 2,4-difluoro(2,4-difluorophenyl), OPh; NPh₂, N-carbazoyl, N(C₆H₅^{t}Bu)₂; or

R^{X8A} and one of the residues R⁷ in ortho position to the nitrogen atom, may form together a ring, wherein the ring is formed via a single bond, via a C₁-C₃ alkyl group which is optionally substituted by a C₁-C₂₅ alkyl group, preferably by a C₁-C₈ alkyl group, more preferably by a C₁-C₄ alkyl group, via an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group, via an unsubstituted or substituted C₂ alkenyl group, via a group NR′, via O, via a group POOR′ or via a unsubstituted or substituted P—C₆-C₃₀ aryl group, preferably unsubstituted or substituted P—C₆-C₁₀ aryl group, more preferably unsubstituted or substituted P—C₆ aryl group, most preferably unsubstituted P—C₆ aryl group; preferably via a single bond,

wherein

R′ represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group.

Examples for compounds of class 6, wherein R^{X8A} and one of the residues R⁷ in ortho position to the nitrogen atom, may form together a ring are the following compounds:

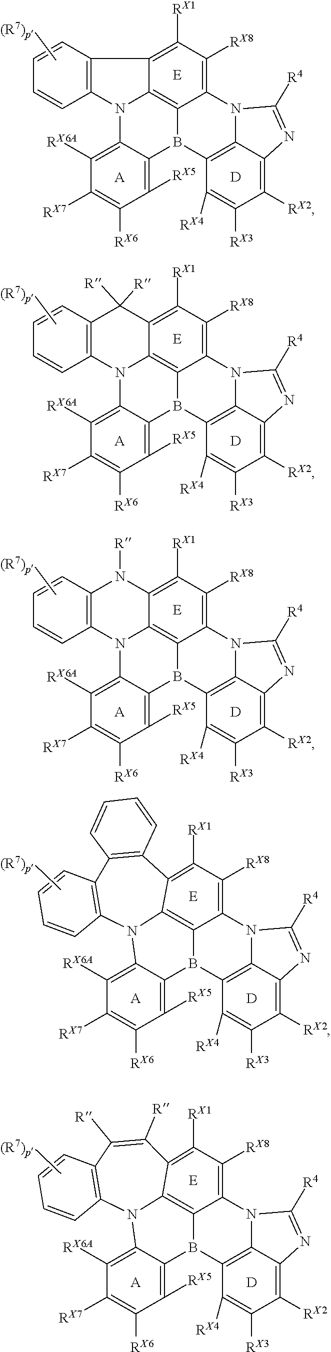

wherein

p′ is 0, 1, 2, 3 or 4, preferably 0, 1 or 2, more preferably 0 or 1;

R″ each independently represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group;

and the other groups, residues and indices are as defined in formula (XII).

Examples for compounds of class 6 are compounds of the following formula (O), wherein the residues R¹, R^{X1}, R^{X2}, R^{X3}, R^{X6} and R⁷ are defined above.

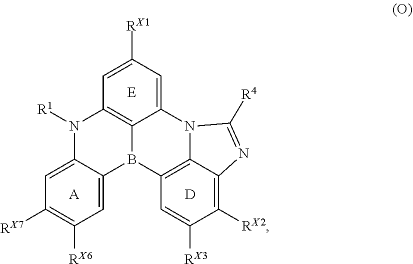

Preferred compounds (O) are mentioned in the following table, wherein Groups 1, 2, 4, 5, 6, 7, 8 and 10 are defined as follows:

| R⁴ | R^{X7} | R^{X6} | R¹ | R^{X1} | R^{X3} | R^{X2} |
|---|---|---|---|---|---|---|
| Ph | H | H | Ph | H | H | H |
| Group1 | H | H | Ph | H | H | H |
| Group4 | H | H | Ph | H | H | H |
| CF3 | H | H | Ph | H | H | H |
| tBu | H | H | Ph | H | H | H |
| Ph | H | tBu | Group1 | H | H | H |
| Group1 | H | tBu | Group1 | H | H | H |
| Group4 | H | tBu | Group1 | H | H | H |
| CF3 | H | tBu | Group1 | H | H | H |
| tBu | H | tBu | Group1 | H | H | H |
| Ph | H | H | Ph | H | H | F |
| Group1 | H | H | Ph | H | H | F |
| Group4 | H | H | Ph | H | H | F |
| CF3 | H | H | Ph | H | H | F |
| tBu | H | H | Ph | H | H | F |

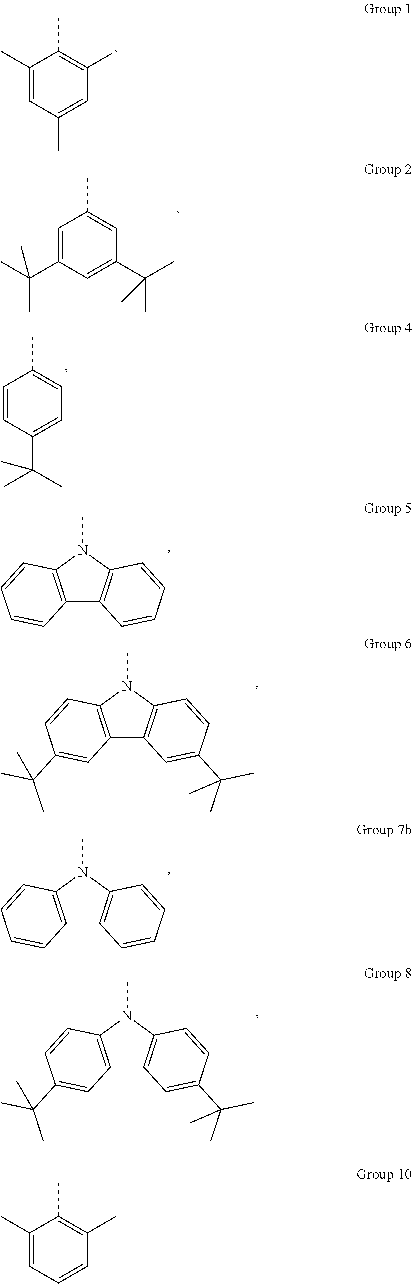

| R⁴ | R^{X7} | R^{X6} | R¹ | R^{X1} | R^{X3} | R^{X2} |
|---|---|---|---|---|---|---|
| Ph | H | H | Ph | Me | H | H |
| Group1 | H | H | Ph | Me | H | H |
| Group4 | H | H | Ph | Me | H | H |
| CF3 | H | H | Ph | Me | H | H |
| tBu | H | H | Ph | Me | H | H |
| Ph | H | tBu | Group1 | Me | H | H |
| Group1 | H | tBu | Group1 | Me | H | H |
| Group4 | H | tBu | Group1 | Me | H | H |
| CF3 | H | tBu | Group1 | Me | H | H |
| tBu | H | tBu | Group1 | Me | H | H |
| Ph | H | H | Ph | Me | H | F |
| Group1 | H | H | Ph | Me | H | F |
| Group4 | H | H | Ph | Me | H | F |
| CF3 | H | H | Ph | Me | H | F |
| tBu | H | H | Ph | Me | H | F |
| Ph | H | H | Ph | Group5 | H | H |
| Group1 | H | H | Ph | Group5 | H | H |
| Group4 | H | H | Ph | Group5 | H | H |
| CF3 | H | H | Ph | Group5 | H | H |
| tBu | H | H | Ph | Group5 | H | H |
| Ph | H | tBu | Group1 | Group5 | H | H |
| Group1 | H | tBu | Group1 | Group5 | H | H |
| Group4 | H | tBu | Group1 | Group5 | H | H |
| CF3 | H | tBu | Group1 | Group5 | H | H |
| tBu | H | tBu | Group1 | Group5 | H | H |
| Ph | H | H | Ph | Group5 | H | F |
| Group1 | H | H | Ph | Group5 | H | F |
| Group4 | H | H | Ph | Group5 | H | F |
| CF3 | H | H | Ph | Group5 | H | F |
| tBu | H | H | Ph | Group5 | H | F |
| Group 1 | Group 5 | H | Group4 | H | H | H |
| tBu | Ph | H | Group2 | H | H | H |
| tBu | Group 5 | H | Group4 | H | H | H |
| Group 10 | Group 5 | H | Group4 | H | H | H |
| tBu | Ph | H | Group4 | H | H | H |
| Group 1 | Ph | H | Group2 | H | H | H |
| Group 1 | Group 5 | H | Group4 | H | H | H |
| Group 10 | Ph | H | Group2 | H | H | H |
| Group 10 | Group 5 | H | Group4 | H | H | H |
| Ph | H | H | Ph | Group6 | H | H |
| Group1 | H | H | Ph | Group6 | H | H |
| Group4 | H | H | Ph | Group6 | H | H |
| CF3 | H | H | Ph | Group6 | H | H |
| tBu | H | H | Ph | Group6 | H | H |
| Ph | H | tBu | Group1 | Group6 | H | H |
| Group1 | H | tBu | Group1 | Group6 | H | H |
| Group4 | H | tBu | Group1 | Group6 | H | H |
| CF3 | H | tBu | Group1 | Group6 | H | H |
| tBu | H | tBu | Group1 | Group6 | H | H |
| Ph | H | H | Ph | Group6 | H | F |
| Group1 | H | H | Ph | Group6 | H | F |
| Group4 | H | H | Ph | Group6 | H | F |
| CF3 | H | H | Ph | Group6 | H | F |
| tBu | H | H | Ph | Group6 | H | F |
| Ph | H | H | Ph | Group7b | H | H |
| Group1 | H | H | Ph | Group7b | H | H |
| Group4 | H | H | Ph | Group7b | H | H |
| CF3 | H | H | Ph | Group7b | H | H |
| tBu | H | H | Ph | Group7b | H | H |
| Ph | H | tBu | Group1 | Group7b | H | H |
| Group1 | H | tBu | Group1 | Group7b | H | H |
| Group4 | H | tBu | Group1 | Group7b | H | H |
| CF3 | H | tBu | Group1 | Group7b | H | H |
| tBu | H | tBu | Group1 | Group7b | H | H |
| Ph | H | H | Ph | Group7b | H | F |
| Group1 | H | H | Ph | Group7b | H | F |
| Group4 | H | H | Ph | Group7b | H | F |
| CF3 | H | H | Ph | Group7b | H | F |
| tBu | H | H | Ph | Group7b | H | F |
| Ph | H | H | Ph | Group8 | H | H |
| Group1 | H | H | Ph | Group8 | H | H |
| Group4 | H | H | Ph | Group8 | H | H |
| CF3 | H | H | Ph | Group8 | H | H |
| tBu | H | H | Ph | Group8 | H | H |
| Ph | H | tBu | Group1 | Group8 | H | H |
| Group1 | H | tBu | Group1 | Group8 | H | H |
| Group4 | H | tBu | Group1 | Group8 | H | H |
| CF3 | H | tBu | Group1 | Group8 | H | H |
| tBu | H | tBu | Group1 | Group8 | H | H |
| Ph | H | H | Ph | Group8 | H | F |
| Group1 | H | H | Ph | Group8 | H | F |
| Group4 | H | H | Ph | Group8 | H | F |
| CF3 | H | H | Ph | Group8 | H | F |
| tBu | H | H | Ph | Group8 | H | F |
| Ph | H | H | Ph | F | H | H |
| Group1 | H | H | Ph | F | H | H |
| Group4 | H | H | Ph | F | H | H |
| CF3 | H | H | Ph | F | H | H |
| tBu | H | H | Ph | F | H | H |
| Ph | H | tBu | Group1 | F | H | H |
| Group1 | H | tBu | Group1 | F | H | H |
| Group4 | H | tBu | Group1 | F | H | H |
| CF3 | H | tBu | Group1 | F | H | H |
| tBu | H | tBu | Group1 | F | H | H |
| Ph | H | H | Ph | F | H | F |
| Group1 | H | H | Ph | F | H | F |
| Group4 | H | H | Ph | F | H | F |
| CF3 | H | H | Ph | F | H | F |
| tBu | H | H | Ph | F | H | F |
| Ph | Group5 | H | Ph | H | H | H |
| CF3 | Group5 | H | Ph | H | H | H |
| tBu | Group5 | H | Ph | H | H | H |
| Group1 | Group5 | H | Ph | H | H | H |
| Group4 | Group5 | H | Ph | H | H | H |
| Ph | Group6 | H | Ph | H | H | H |
| CF3 | Group6 | H | Ph | H | H | H |
| tBu | Group6 | H | Ph | H | H | H |
| Group1 | Group6 | H | Ph | H | H | H |
| Group4 | Group6 | H | Ph | H | H | H |
| Ph | Group7b | H | Ph | H | H | H |
| CF3 | Group7b | H | Ph | H | H | H |
| tBu | Group7b | H | Ph | H | H | H |
| Group1 | Group7b | H | Ph | H | H | H |
| Group4 | Group7b | H | Ph | H | H | H |
| Ph | Group8 | H | Ph | H | H | H |
| CF3 | Group8 | H | Ph | H | H | H |
| tBu | Group8 | H | Ph | H | H | H |
| Group1 | Group8 | H | Ph | H | H | H |
| Group4 | Group8 | H | Ph | H | H | H |
| Ph | H | Ph | m biphenyl | H | H | H |
| CF3 | H | Ph | m biphenyl | H | H | H |
| tBu | H | Ph | m biphenyl | H | H | H |
| Group1 | H | Ph | m biphenyl | H | H | H |
| Group4 | H | Ph | m biphenyl | H | H | H |
| Ph | Group5 | H | Ph | Me | H | H |
| CF3 | Group5 | H | Ph | Me | H | H |
| tBu | Group5 | H | Ph | Me | H | H |
| Group1 | Group5 | H | Ph | Me | H | H |
| Group4 | Group5 | H | Ph | Me | H | H |
| Ph | Group6 | H | Ph | Me | H | H |
| CF3 | Group6 | H | Ph | Me | H | H |
| tBu | Group6 | H | Ph | Me | H | H |
| Group1 | Group6 | H | Ph | Me | H | H |
| Group4 | Group6 | H | Ph | Me | H | H |
| Ph | Group7b | H | Ph | Me | H | H |
| CF3 | Group7b | H | Ph | Me | H | H |
| tBu | Group7b | H | Ph | Me | H | H |
| Group1 | Group7b | H | Ph | Me | H | H |
| Group4 | Group7b | H | Ph | Me | H | H |
| Ph | Group8 | H | Ph | Me | H | H |
| CF3 | Group8 | H | Ph | Me | H | H |
| tBu | Group8 | H | Ph | Me | H | H |
| Group1 | Group8 | H | Ph | Me | H | H |
| Group4 | Group8 | H | Ph | Me | H | H |
| Ph | H | Ph | m biphenyl | Me | H | H |
| CF3 | H | Ph | m biphenyl | Me | H | H |
| tBu | H | Ph | m biphenyl | Me | H | H |
| Group1 | H | Ph | m biphenyl | Me | H | H |
| Group4 | H | Ph | m biphenyl | Me | H | H |

Further preferred compounds (O) are mentioned in the following table:

| R⁴ | R^{X7} | R^{X6} | R¹ | R^{X1} | R^{X3} | R^{X2} |
|---|---|---|---|---|---|---|
| Group10 | H | H | Ph | H | H | H |
| Ph | H | tBu | Group10 | H | H | H |
| Group10 | H | tBu | Group10 | H | H | H |
| Group4 | H | tBu | Group10 | H | H | H |
| CF3 | H | tBu | Group10 | H | H | H |
| tBu | H | tBu | Group10 | H | H | H |
| Group10 | H | H | Ph | H | H | F |
| Group10 | H | H | Ph | Me | H | H |
| Ph | H | tBu | Group10 | Me | H | H |
| Group10 | H | tBu | Group10 | Me | H | H |
| Group4 | H | tBu | Group10 | Me | H | H |
| CF3 | H | tBu | Group10 | Me | H | H |
| tBu | H | tBu | Group10 | Me | H | H |
| Group10 | H | H | Ph | Me | H | F |
| Group10 | H | H | Ph | Group5 | H | H |
| Ph | H | tBu | Group10 | Group5 | H | H |
| Group10 | H | tBu | Group10 | Group5 | H | H |
| Group4 | H | tBu | Group10 | Group5 | H | H |
| CF3 | H | tBu | Group10 | Group5 | H | H |
| tBu | H | tBu | Group10 | Group5 | H | H |
| Group10 | H | H | Ph | Group5 | H | F |
| Group10 | H | H | Ph | Group6 | H | H |
| Ph | H | tBu | Group10 | Group6 | H | H |
| Group10 | H | tBu | Group10 | Group6 | H | H |
| Group4 | H | tBu | Group10 | Group6 | H | H |
| CF3 | H | tBu | Group10 | Group6 | H | H |
| tBu | H | tBu | Group10 | Group6 | H | H |
| Group10 | H | H | Ph | Group6 | H | F |
| Group10 | H | H | Ph | Group7b | H | H |
| Ph | H | tBu | Group10 | Group7b | H | H |
| Group10 | H | tBu | Group10 | Group7b | H | H |
| Group4 | H | tBu | Group10 | Group7b | H | H |
| CF3 | H | tBu | Group10 | Group7b | H | H |
| tBu | H | tBu | Group10 | Group7b | H | H |
| Group10 | H | H | Ph | Group7b | H | F |
| Group10 | H | H | Ph | Group8 | H | H |
| Ph | H | tBu | Group10 | Group8 | H | H |
| Group10 | H | tBu | Group10 | Group8 | H | H |
| Group4 | H | tBu | Group10 | Group8 | H | H |
| CF3 | H | tBu | Group10 | Group8 | H | H |
| tBu | H | tBu | Group10 | Group8 | H | H |
| Group10 | H | H | Ph | Group8 | H | F |
| Group10 | H | H | Ph | F | H | H |
| Ph | H | tBu | Group10 | F | H | H |
| Group10 | H | tBu | Group10 | F | H | H |
| Group4 | H | tBu | Group10 | F | H | H |
| CF3 | H | tBu | Group10 | F | H | H |
| tBu | H | tBu | Group10 | F | H | H |
| Group10 | H | H | Ph | F | H | F |
| Group10 | Group5 | H | Ph | H | H | H |
| Group10 | Group6 | H | Ph | H | H | H |
| Group10 | Group7b | H | Ph | H | H | H |
| Group10 | Group8 | H | Ph | H | H | H |
| Group10 | H | Ph | m biphenyl | H | H | H |
| Group10 | Group5 | H | Ph | Me | H | H |
| Group10 | Group6 | H | Ph | Me | H | H |
| Group10 | Group7b | H | Ph | Me | H | H |
| Group10 | Group8 | H | Ph | Me | H | H |
| Group10 | H | Ph | m biphenyl | Me | H | H |

Further examples for compounds of class 6 are compounds of the following formula (P), wherein the residues R⁴, R⁷, R^{X1}, R^{X2}, R^{X3} and R^{X6} are defined above.

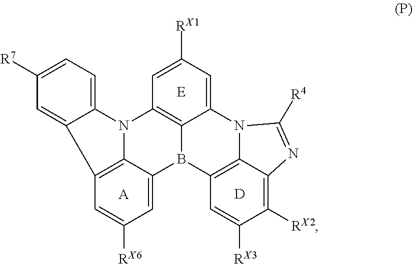

Preferred compounds (P) are mentioned in the following table, wherein Groups 1, 4 and 10 are defined as follows:

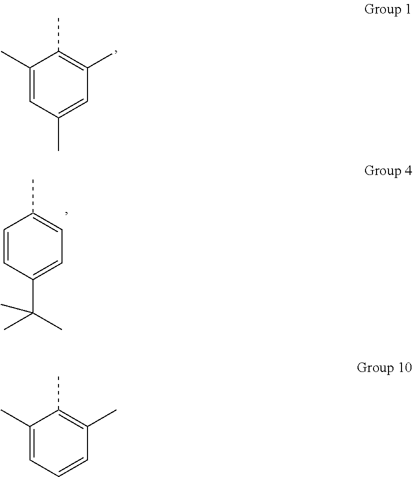

| R⁴ | R^{X6} | R⁷ | R^{X1} | R^{X3} | R^{X2} |
|---|---|---|---|---|---|
| Ph | H | H | H | H | H |
| Group1 | H | H | H | H | H |
| Group4 | H | H | H | H | H |
| CF3 | H | H | H | H | H |
| tBu | H | H | H | H | H |
| Ph | tBu | tBu | H | H | H |
| Group1 | tBu | tBu | H | H | H |
| Group4 | tBu | tBu | H | H | H |
| CF3 | tBu | tBu | H | H | H |
| tBu | tBu | tBu | H | H | H |
| Ph | H | H | H | H | F |
| Group1 | H | H | H | H | F |
| Group4 | H | H | H | H | F |
| CF3 | H | H | H | H | F |
| tBu | H | H | H | H | F |
| Ph | H | H | Me | H | H |
| Group1 | H | H | Me | H | H |
| Group4 | H | H | Me | H | H |
| CF3 | H | H | Me | H | H |
| tBu | H | H | Me | H | H |
| Ph | tBu | tBu | Me | H | H |
| Group1 | tBu | tBu | Me | H | H |
| Group4 | tBu | tBu | Me | H | H |
| CF3 | tBu | tBu | Me | H | H |
| tBu | tBu | tBu | Me | H | H |
| Ph | H | H | Me | H | F |
| Group1 | H | H | Me | H | F |
| Group4 | H | H | Me | H | F |
| CF3 | H | H | Me | H | F |
| tBu | H | H | Me | H | F |

Further preferred compounds (P) are mentioned in the following table:

| R⁴ | R^{X6} | R⁷ | R^{X1} | R^{X3} | R^{X2} |
|---|---|---|---|---|---|
| Ph | H | H | H | H | F |
| CF3 | H | H | H | H | F |
| tBu | H | H | H | H | F |
| Group1 | H | H | H | H | F |
| Group4 | H | H | H | H | F |
| Ph | tBu | H | H | H | F |
| CF3 | tBu | H | H | H | F |
| tBu | tBu | H | H | H | F |
| Group1 | tBu | H | H | H | F |
| Group4 | tBu | H | H | H | F |
| Ph | tBu | tBu | H | H | F |
| CF3 | tBu | tBu | H | H | F |
| tBu | tBu | tBu | H | H | F |
| Group1 | tBu | tBu | H | H | F |
| Group4 | tBu | tBu | H | H | F |

Further preferred compounds (P) are mentioned in the following table:

| R⁴ | R^{X6} | R⁷ | R^{X1} | R^{X3} | R^{X2} |
|---|---|---|---|---|---|
| Group10 | H | H | H | H | H |
| Group10 | tBu | tBu | H | H | H |
| Group10 | H | H | H | H | F |
| Group10 | H | H | Me | H | H |
| Group10 | tBu | tBu | Me | H | H |
| Group10 | H | H | Me | H | F |

Further examples for compounds of class 6 are compounds of the following formula (Q), wherein the residues R⁴, R⁷, R^{X1}, R^{X2}, R^{X3} and R^{X6} are defined above.

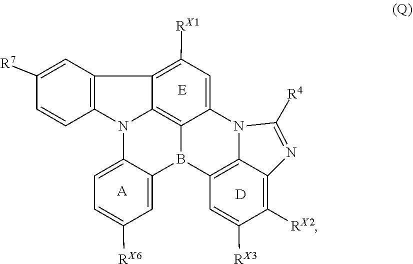

Preferred compounds (Q) are mentioned in the following table, wherein Groups 1, 4 and 10 are defined as follows:

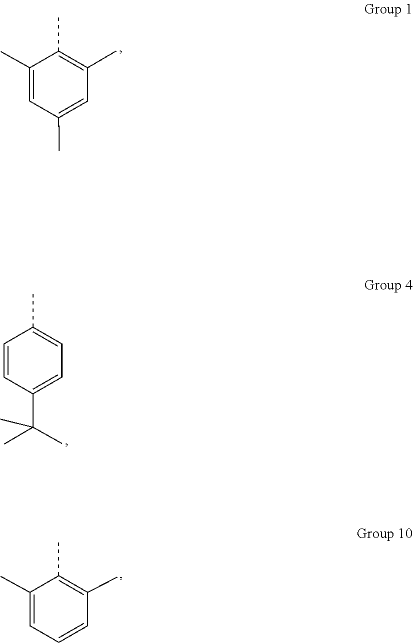

| R⁴ | R^{X6} | R⁷ | R^{X1} | R^{X3} | R^{X2} |
|---|---|---|---|---|---|
| Ph | H | H | H | H | H |
| CF3 | H | H | H | H | H |
| tBu | H | H | H | H | H |
| Group1 | H | H | H | H | H |
| Group4 | H | H | H | H | H |
| Ph | tBu | H | H | H | H |
| CF3 | tBu | H | H | H | H |
| tBu | tBu | H | H | H | H |
| Group1 | tBu | H | H | H | H |
| Group4 | tBu | H | H | H | H |
| Ph | tBu | tBu | H | H | H |
| CF3 | tBu | tBu | H | H | H |
| tBu | tBu | tBu | H | H | H |
| Group1 | tBu | tBu | H | H | H |
| Group4 | tBu | tBu | H | H | H |
| Ph | H | H | Me | H | H |
| CF3 | H | H | Me | H | H |
| tBu | H | H | Me | H | H |
| Group1 | H | H | Me | H | H |
| Group4 | H | H | Me | H | H |
| Ph | tBu | H | Me | H | H |
| CF3 | tBu | H | Me | H | H |
| tBu | tBu | H | Me | H | H |
| Group1 | tBu | H | Me | H | H |
| Group4 | tBu | H | Me | H | H |
| Ph | tBu | tBu | Me | H | H |
| CF3 | tBu | tBu | Me | H | H |
| tBu | tBu | tBu | Me | H | H |
| Group1 | tBu | tBu | Me | H | H |
| Group4 | tBu | tBu | Me | H | H |
| Ph | H | H | H | tBu | H |
| CF3 | H | H | H | tBu | H |
| tBu | H | H | H | tBu | H |
| Group1 | H | H | H | tBu | H |
| Group4 | H | H | H | tBu | H |
| Ph | tBu | H | H | tBu | H |
| CF3 | tBu | H | H | tBu | H |
| tBu | tBu | H | H | tBu | H |
| Group1 | tBu | H | H | tBu | H |
| Group4 | tBu | H | H | tBu | H |
| Ph | tBu | tBu | H | tBu | H |
| CF3 | tBu | tBu | H | tBu | H |
| tBu | tBu | tBu | H | tBu | H |
| Group1 | tBu | tBu | H | tBu | H |
| Group4 | tBu | tBu | H | tBu | H |

Further preferred compounds (Q) are mentioned in the following table:

| R⁴ | R^{X6} | R⁷ | R^{X1} | R^{X3} | R^{X2} |
|---|---|---|---|---|---|
| Group10 | H | H | H | H | H |
| Group10 | tBu | H | H | H | H |
| Group10 | tBu | tBu | H | H | H |
| Group10 | H | H | H | H | H |
| Group10 | tBu | H | H | H | H |
| Group10 | tBu | tBu | H | H | H |
| tBu | tBu | H | H | H | H |
| tBu | tBu | tBu | H | H | H |
| Group10 | H | H | Me | H | H |
| Group10 | tBu | H | Me | H | H |
| Group10 | tBu | tBu | Me | H | H |
| Group10 | H | H | H | tBu | H |
| Group10 | tBu | H | H | tBu | H |
| Group10 | tBu | tBu | H | tBu | H |
| Group10 | H | H | H | H | F |
| Group10 | tBu | H | H | H | F |
| Group10 | tBu | tBu | H | H | F |

Further examples for compounds of class 6 are compounds of the following formula (R), wherein the residues R⁴, R⁷, R^{X1}, R^{X2}, R^{X3} and R^{X6} are defined above.

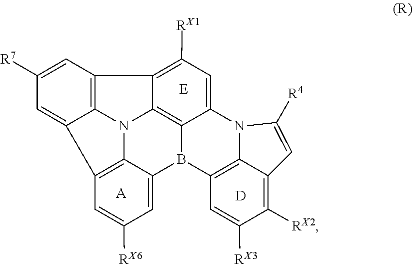

Preferred compounds (R) are mentioned in the following table, wherein Groups 1, 4 and 10 are defined as follows:

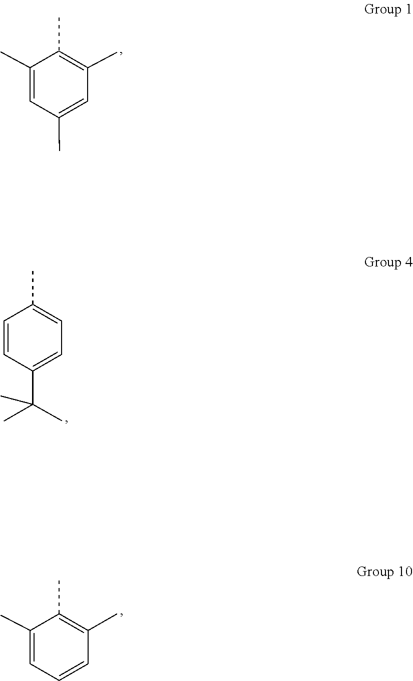

| R⁴ | R^{X6} | R⁷ | R^{X1} | R^{X3} | R^{X2} |
|---|---|---|---|---|---|
| Ph | H | H | H | H | H |
| CF3 | H | H | H | H | H |
| tBu | H | H | H | H | H |
| Group1 | H | H | H | H | H |
| Group4 | H | H | H | H | H |
| Ph | tBu | H | H | H | H |
| CF3 | tBu | H | H | H | H |
| tBu | tBu | H | H | H | H |
| Group1 | tBu | H | H | H | H |
| Group4 | tBu | H | H | H | H |
| Ph | tBu | tBu | H | H | H |
| CF3 | tBu | tBu | H | H | H |
| tBu | tBu | tBu | H | H | H |
| Group1 | tBu | tBu | H | H | H |
| Group4 | tBu | tBu | H | H | H |

Further preferred compounds (R) are mentioned in the following table:

| R⁴ | R^{X6} | R⁷ | R^{X1} | R^{X3} | R^{X2} |
|---|---|---|---|---|---|
| Group10 | H | H | H | H | H |
| Group10 | tBu | H | H | H | H |
| Group10 | tBu | tBu | H | H | H |

The compounds of formula

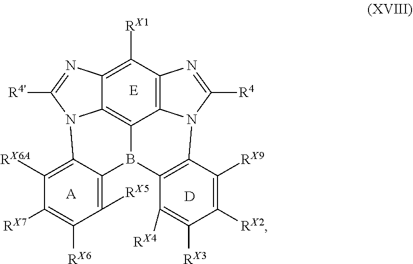

### Class 7

In the compounds of class 7 (formula (XVIII)), the groups, residues and indices R⁴, R^{4′}, R^{X1}, R^{X2}, R^{X3}, R^{X4}, R^{X5}, R^{X6}, R^{X7}, R^{X6A} and R^{X9} are defined above.

In the case that R^{X6A} and R^{X7}, R^{X6} and R^{X7}, R^{X5} and R^{X6}, R^{X2} and R^{X9}, R^{X2} and R^{X3} and/or R^{X3} and R^{X4}, in the compounds of class 7 may form together an unsubstituted or substituted ring, the following rings (a) and (b) are formed:

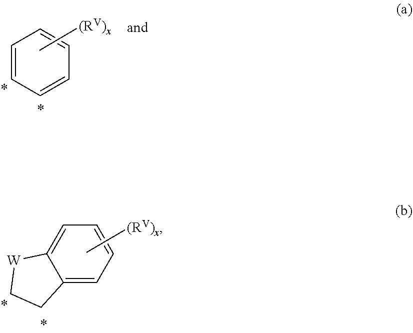

wherein

R^{V} represents H, a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group;

x represents 0, 1, 2, 3 or 4, preferably 0, 1 or 2, more preferably 0; and

W represents CR′″₂, O, S or NR^{IV};

R′″ represents a C₁-C₂₅ alkyl group, preferably a C₁-C₈ alkyl group, more preferably a C₁-C₄ alkyl group;

R^{IV} represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group; and

each * represents a point of attachment to ring A or D.

Most preferably, R^{X1}, R^{X4} and R^{X5} in the compounds of class 7 are H.

Most preferably, R⁴ and R^{4′} in the compounds of class 7 each independently represents tert-butyl, CF₃, unsubstituted phenyl, p-tert-butyl-phenyl, xylyl or mesityl.

Most preferably, R^{X6A}, R^{X7}, R^{X6}, R^{X3}, R^{X2} and R^{X9} each independently represents H, methyl, ethyl, iso-propyl, sec-propyl, n-butyl, tert-butyl, —C(Me)₂Et, unsubstituted phenyl, p-tert-butyl-phenyl, mesityl, xylyl, O-methyl-phenyl or unsubstituted or substituted biphenyl, preferably unsubstituted biphenyl.

Most preferably, R^{X4} and R^{X5} are H, further most preferably, R^{X4}, R^{X5}, R^{X6A} and R^{X9} are H.

Most preferably, R^{X1} in the compounds of class 7 is H, methyl, ethyl, n-butyl, unsubstituted phenyl, —O-phenyl, —NPh₂, N-carbazolyl, —N(C₆H₅^{t}bu)₂ or —OMe, most preferably H.

Examples for compounds of class 7 are compounds of the following formula (S), wherein the residues R⁴, R^{4′}, R^{X3} and R^{X6} are defined above.

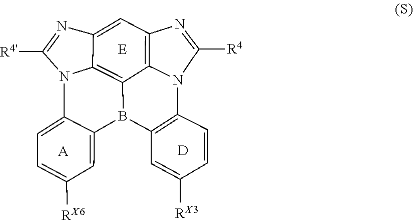

Preferred compounds (S) are mentioned in the following table, wherein Groups 1 and 4 are defined as follows:

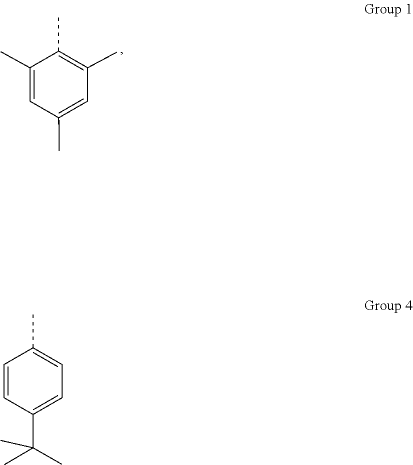

| R⁴ | R^{4′} | R^{X3} | R^{X6} |
|---|---|---|---|
| Ph | Ph | H | H |
| tBu | tBu | H | H |
| Group1 | Group1 | H | H |
| Group4 | Group4 | H | H |
| CF3 | CF3 | H | H |
| Ph | Ph | tBu | tBu |
| tBu | tBu | tBu | tBu |
| Group1 | Group1 | tBu | tBu |
| Group4 | Group4 | tBu | tBu |
| CF3 | CF3 | tBu | tBu |

Further examples for compounds of class 7 are compounds of the following formula (T), wherein the residues R⁴, R^{4′}, R^{X3} and R^{X6} are defined above.

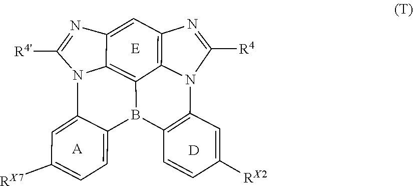

Preferred compounds (T) are mentioned in the following table, wherein Groups 1, 4, 5, 6, 7, 8 and 10 are defined as follows:

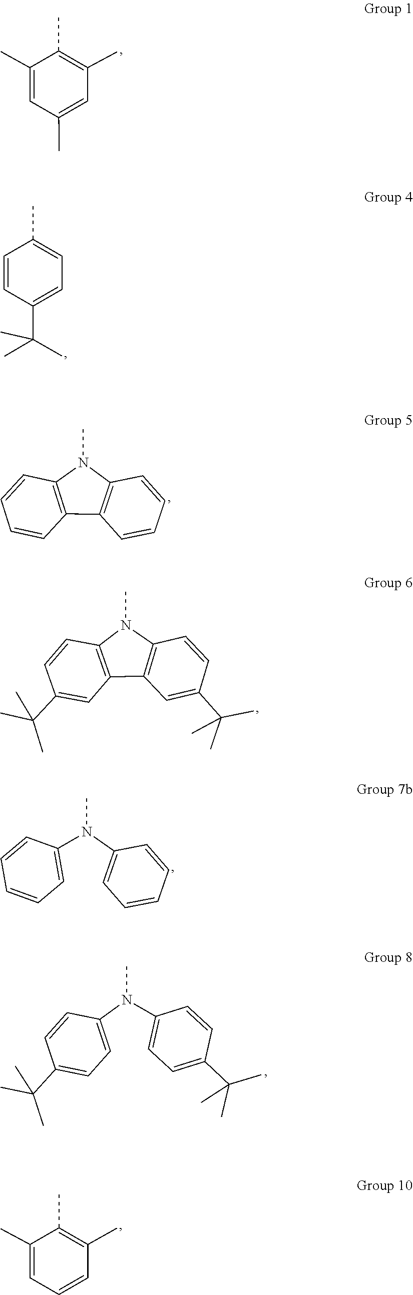

| R⁴ | R^{4′} | R^{X2} | R^{X7} |
|---|---|---|---|
| Ph | Ph | Group5 | Group5 |
| tBu | tBu | Group5 | Group5 |
| Group1 | Group1 | Group5 | Group5 |
| Group4 | Group4 | Group5 | Group5 |
| CF3 | CF3 | Group5 | Group5 |
| Ph | Ph | Group6 | Group6 |
| tBu | tBu | Group6 | Group6 |
| Group1 | Group1 | Group6 | Group6 |
| Group4 | Group4 | Group6 | Group6 |
| CF3 | CF3 | Group6 | Group6 |
| Ph | Ph | Group7b | Group7b |
| tBu | tBu | Group7b | Group7b |
| Group1 | Group1 | Group7b | Group7b |
| Group4 | Group4 | Group7b | Group7b |
| CF3 | CF3 | Group7b | Group7b |
| Ph | Ph | Group8 | Group8 |
| tBu | tBu | Group8 | Group8 |
| Group1 | Group1 | Group8 | Group8 |
| Group4 | Group4 | Group8 | Group8 |
| CF3 | CF3 | Group8 | Group8 |
| Group10 | Group10 | Group5 | Group5 |
| Group10 | Group10 | Group6 | Group6 |
| Group10 | Group10 | Group7b | Group7b |
| Group10 | Group10 | Group8 | Group8 |

The compounds of formula

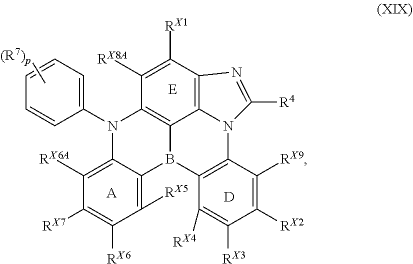

### Class 8

In the compounds of class 8 (formula (XIX)), the groups, residues and indices R⁴, R⁷, R^{X1}, R^{X2}, R^{X3}, R^{X4}, R^{X5}, R^{X6}, R^{X7}, R^{X6A}, R^{X9} and R^{X8A} are defined above.

In the case that R^{X6A} and R^{X7}, R^{X6} and R^{X7}, R^{X5} and R^{X6}, R^{X2} and R^{X3}, R^{X3} and R^{X4}, R^{X2} and R^{X9}, R^{X1} and R^{X8A} and/or two adjacent residues R⁷, in the compounds of class 8 may form together an unsubstituted or substituted ring, the following rings (a) and (b) are formed:

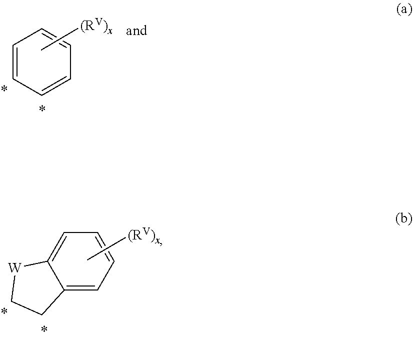

wherein

R^{V} represents H, a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group;

x represents 0, 1, 2, 3 or 4, preferably 0, 1 or 2, more preferably 0; and W represents CR′″₂, O, S or NR^{IV};

R′″ represents a C₁-C₂₅ alkyl group, preferably a C₁-C₈ alkyl group, more preferably a C₁-C₄ alkyl group;

R^{IV} represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group; and

each * represents a point of attachment to ring A, D or E, or to the phenyl ring bearing the R⁷-substituent(s).

Most preferably, R⁴ in the compounds of class 8 represents methyl, ethyl, iso-propyl, sec-propyl, n-butyl, t-butyl, CF₃, —C(Me)₂C₂H₅, unsubstituted phenyl, p-tert-butyl-phenyl, mesityl, xylyl, O-methyl-phenyl or unsubstituted or substituted biphenyl, preferably unsubstituted biphenyl.

Most preferably, R^{X2}, R^{X3}, R^{X4} and R^{X9} in the compounds of class 8 each independently represents H, methyl, ethyl, iso-propyl, sec-propyl, n-butyl, tert-butyl, —C(Me)₂C₂H₅, unsubstituted phenyl, p-tert-butyl-phenyl, mesityl, xylyl, O-methyl-phenyl, unsubstituted or substituted biphenyl, preferably unsubstituted biphenyl or 2,4-difluoro(2,4-difluorophenyl).

Most preferably, R^{X4} and R^{X5} in the compounds of class 8 are H. Further most preferably, R^{X4}, R^{X5} and R^{X8A} in the compounds of class 8 are H.

Most preferably, R^{X6}, R^{X6A}, R^{X7} and R⁷ in the compounds of class 8 each independently represents H, methyl, ethyl, iso-propyl, sec-propyl, n-butyl, tert-butyl, —C(Me)₂C₂H₅, unsubstituted phenyl, p-tert-butyl-phenyl, mesityl, xylyl, O-methyl-phenyl, unsubstituted or substituted biphenyl, preferably unsubstituted biphenyl or 2,4-difluoro(2,4-difluorophenyl); or R^{X6A} and one of the residues R⁷ in ortho position to the nitrogen atom, may form together a ring, wherein the ring is formed via a single bond, via a C₁-C₃ alkyl group which is optionally substituted by a C₁-C₂₅ alkyl group, preferably by a C₁-C₈ alkyl group, more preferably by a C₁-C₄ alkyl group, via an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group, via an unsubstituted or substituted C₂ alkenyl group, via a group NR′, via O, via a group POOR′ or via a unsubstituted or substituted P—C₆-C₃₀ aryl group, preferably unsubstituted or substituted P—C₆-C₁₀ aryl group, more preferably unsubstituted or substituted P—C₆ aryl group, most preferably unsubstituted P—C₆ aryl group; preferably via a single bond,

wherein

R′ represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group.

Examples for compounds of class 8, wherein R^{X6A} and one of the residues R⁷ in ortho position to the nitrogen atom, form together a ring are the following compounds:

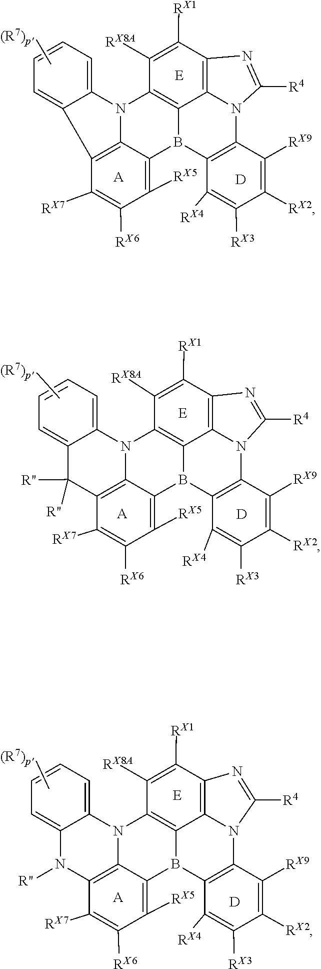

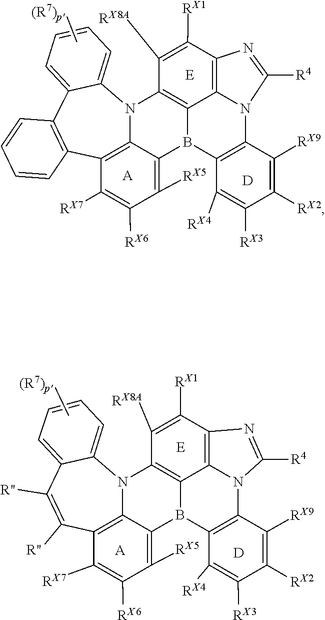

wherein

wherein p′ is 0, 1, 2, 3 or 4, preferably 0, 1 or 2, more preferably 0 or 1;

R″ each independently represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group;

and the other groups, residues and indices are as defined in formula (XV).

R^{X1} and R^{X8A} most preferably each independently represents H, methyl, ethyl, iso-propyl, sec-propyl, n-butyl, tert-butyl, —C(Me)₂Et, F, unsubstituted phenyl, —NPh₂, N(C₆H₅tBu)₂, N-carbazol, N-tBu-carbazol, —OMe or —OPh.

Most preferably, R^{X8A} is H.

Examples for compounds of class 8 are compounds of the following formula (U), wherein the residues R⁴, R^{X3}, R^{X6} and R^{X7} are defined above and R^{7a} and R^{7b} are each independently defined as R⁷ mentioned above.

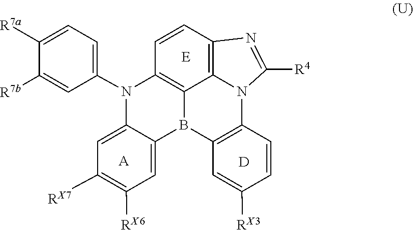

Preferred compounds (U) are mentioned in the following table, wherein Groups 1, 4 and 10 are defined as follows:

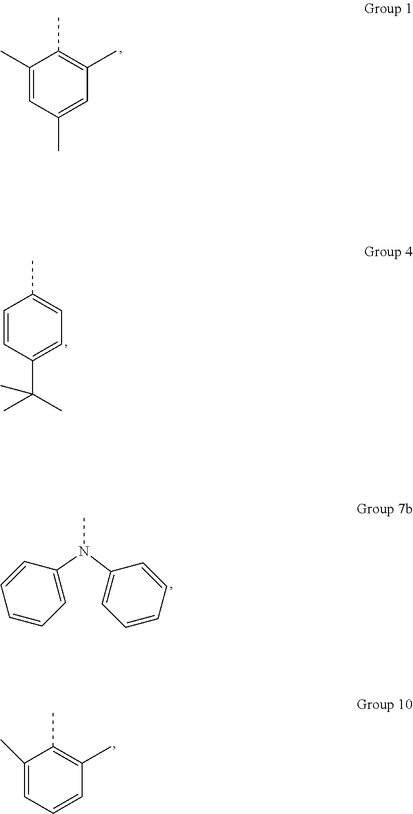

| R⁴ | R^{X3} | R^{X6} | R^{X7} | R^{7a} | R^{7b} |
|---|---|---|---|---|---|
| Ph | H | H | H | H | H |
| tBu | H | H | H | H | H |
| Group1 | H | H | H | H | H |
| Group4 | H | H | H | H | H |
| CF3 | H | H | H | H | H |
| Ph | tBu | H | H | H | H |
| tBu | tBu | H | H | H | H |
| Group1 | tBu | H | H | H | H |
| Group4 | tBu | H | H | H | H |
| CF3 | tBu | H | H | H | H |
| Ph | H | tBu | H | tBu | H |
| tBu | H | tBu | H | tBu | H |
| Group1 | H | tBu | H | tBu | H |
| Group4 | H | tBu | H | tBu | H |
| CF3 | H | tBu | H | tBu | H |
| Ph | H | H | Group7b | H | H |
| tBu | H | H | Group7b | H | H |
| Group1 | H | H | Group7b | H | H |
| Group4 | H | H | Group7b | H | H |
| CF3 | H | H | Group7b | H | H |
| Ph | H | H | Group7b | H | Group7b |
| tBu | H | H | Group7b | H | Group7b |
| Group1 | H | H | Group7b | H | Group7b |
| Group4 | H | H | Group7b | H | Group7b |
| CF3 | H | H | Group7b | H | Group7b |
| Ph | H | H | Ph | H | Ph |
| tBu | H | H | Ph | H | Ph |
| Group1 | H | H | Ph | H | Ph |
| Group4 | H | H | Ph | H | Ph |
| CF3 | H | H | Ph | H | Ph |
| Group10 | H | H | H | H | H |
| Group10 | tBu | H | H | H | H |
| Group10 | H | tBu | H | tBu | H |
| Group10 | H | H | Group7b | H | H |
| Group10 | H | H | Group7b | H | Group7b |
| Group10 | H | H | Ph | H | Ph |

The compounds of formula

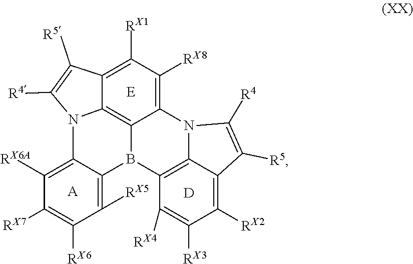

### Class 9

In the compounds of class 9 (formula (XX)), the groups, residues and indices R⁴, R⁵, R^{4′}, R^{5′}, R^{X1}, R^{X2}, R^{X3}, R^{X4}, R^{X5}, R^{X6}, R^{X7}, R^{X8} and R^{X6A} are defined above.

In the case that R^{X6A} and R^{X7}, R^{X6} and R^{X7}, R^{X5} and R^{X6}, R^{X2} and R^{X3}, R^{X3} and R^{X4} and/or R^{X1} and R^{X8}, in the compounds of class 9 may form together an unsubstituted or substituted ring, the following rings (a) and (b) are formed:

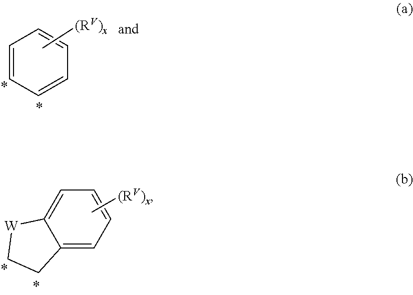

wherein

R^{V} represents H, a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group;

x represents 0, 1, 2, 3 or 4, preferably 0, 1 or 2, more preferably 0; and

W represents CR′″₂, O, S or NR^{IV};

R′″ represents a C₁-C₂₅ alkyl group, preferably a C₁-C₈ alkyl group, more preferably a C₁-C₄ alkyl group; R^{IV} represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C aryl group, most preferably unsubstituted C aryl group; and

each * represents a point of attachment to ring A, D or E.

Most preferably, R⁴, R⁵, R^{4′} and R^{5′} in the compounds of class 9 each independently represents methyl, ethyl, iso-propyl, sec-propyl, n-butyl, —C(Me)₂C₂H₅, tert-butyl, CF₃, SiPh₃, SiBuMe₂, unsubstituted phenyl, p-tert-butyl-phenyl, mesityl, xylyl, O-methyl-phenyl, or unsubstituted or substituted biphenyl, preferably unsubstituted biphenyl;

or

R⁴ and R⁵ may form together an unsubstituted or substituted cyclohexene ring, preferably an unsubstituted cyclohexene ring, and/or

R^{4′} and R^{5′} may form together an unsubstituted or substituted phenyl ring or an unsubstituted or substituted cyclohexene ring, preferably an unsubstituted phenyl ring or an unsubstituted cyclohexene ring.

An example for compounds of class 9, wherein R⁴ and R⁵ may form together an unsubstituted cyclohexene ring is the following compound:

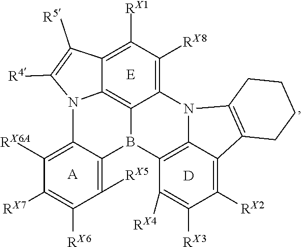

wherein the groups and residues are defined above and below.

Examples for compounds of class 9, wherein R^{4′} and R^{5′} may form together an unsubstituted phenyl ring or an unsubstituted cyclohexene ring are the following compounds:

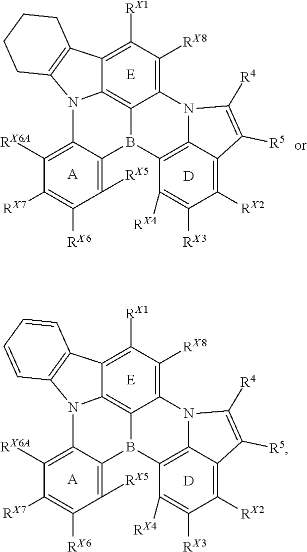

wherein the groups and residues are defined above and below.

Most preferably, R^{X2}, R^{X3} and R^{X4} in the compounds of class 9 each independently represents H, methyl, ethyl, iso-propyl, sec-propyl, n-butyl, tert-butyl, —C(Me)₂C₂H₅, unsubstituted phenyl, p-tert-butyl-phenyl, mesityl, xylyl, O-methyl-phenyl or unsubstituted or substituted biphenyl, preferably unsubstituted biphenyl.

Further most preferably, R^{X4} is H.

Most preferably, R^{X5}, R^{X6}, R^{X6A} and R^{X7} in the compounds of class 9 each independently represents H, methyl, ethyl, iso-propyl, sec-propyl, n-butyl, tert-butyl, —C(Me)₂C₂H₅, unsubstituted phenyl, p-tert-butyl-phenyl, mesityl, xylyl, O-methyl-phenyl or unsubstituted or substituted biphenyl, preferably unsubstituted biphenyl.

Further most preferably, R^{X5} and R^{X6A} are H.

Most preferably, R^{X1} and R^{X8} in the compounds of class 9 each independently represents H, methyl, ethyl, iso-propyl, sec-propyl, n-butyl, —C(Me)₂C₂H₅, F, unsubstituted phenyl, —O— phenyl, —O-methyl, —NPh₂, —N-carbazolyl or —N(C₆H₅^{t}Bu)₂.

Further most preferably, R^{X8} is H.

Further most preferably, R^{X1} is H.

Further most preferably, R^{X7} is H.

Even further most preferably, R^{X1} and R^{X7} are H.

Examples for compounds of class 9 are compounds of the following formula (V), wherein the residues R⁴, R⁵, R^{4′}, R^{5′}, R^{X1}, R^{X3} and R^{X6} are defined above.

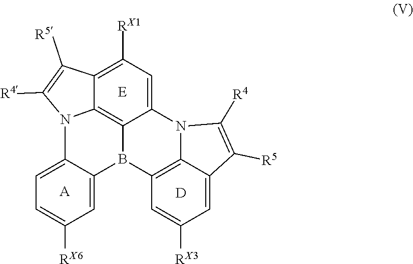

Preferred compounds (V) are mentioned in the following table, wherein Groups 1 and 10 defined as follows:

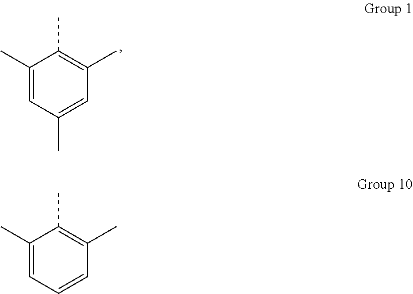

| R⁴ | R^{4′} | R⁵ | R^{5′} | R^{X3} | R^{X6} | R^{X1} |
|---|---|---|---|---|---|---|
| Me | Me | Me | Me | H | H | H |
| Ph | Ph | Ph | Ph | H | H | H |
| Me | Me | group1 | group1 | H | H | H |
| Ph | Ph | group1 | group1 | H | H | H |
| Me | Me | SiPh3 | SiPh3 | H | H | H |
| Ph | Ph | SiPh3 | SiPh3 | H | H | H |
| Me | Me | Me | Me | tBu | tBu | H |
| Ph | Ph | Ph | Ph | tBu | tBu | H |
| Me | Me | group1 | group1 | tBu | tBu | H |
| Ph | Ph | group1 | group1 | tBu | tBu | H |
| Me | Me | SiPh3 | SiPh3 | tBu | tBu | H |
| Ph | Ph | SiPh3 | SiPh3 | tBu | tBu | H |
| Me | Me | Me | Me | tBu | tBu | Me |
| Ph | Ph | Ph | Ph | tBu | tBu | Me |
| Me | Me | group10 | group1 | H | H | H |
| Ph | Ph | group10 | group1 | H | H | H |
| Me | Me | group10 | group1 | tBu | tBu | H |
| Ph | Ph | group10 | group1 | tBu | tBu | H |

Further examples for compounds of class 9 are compounds of the following formula (W), wherein the residues R⁴, R⁵, R^{X1}, R^{X3} and R^{X6} are defined above.

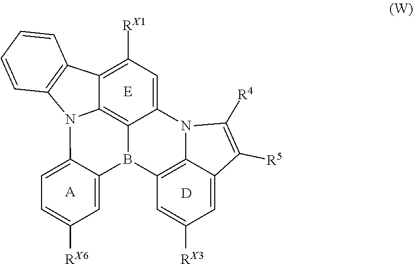

Preferred compounds (W) are mentioned in the following table, wherein Groups 1 and 10 are defined as follows:

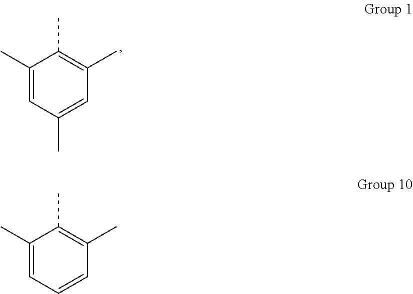

| R⁴ | R⁵ | R^{X3} | R^{X6} | R^{X1} |
|---|---|---|---|---|
| Me | Me | H | H | H |
| Ph | Ph | H | H | H |
| Me | group1 | H | H | H |
| Ph | group1 | H | H | H |
| Me | SiPh3 | H | H | H |
| Ph | SiPh3 | H | H | H |
| Me | Me | tBu | tBu | H |
| Ph | Ph | tBu | tBu | H |
| Me | group1 | tBu | tBu | H |
| Ph | group1 | tBu | tBu | H |
| Me | SiPh3 | tBu | tBu | H |
| Ph | SiPh3 | tBu | tBu | H |
| Me | Me | tBu | tBu | Me |
| Ph | Ph | tBu | tBu | Me |
| Me | group10 | H | H | H |
| Ph | group10 | H | H | H |
| Me | group10 | tBu | tBu | H |
| Ph | group10 | tBu | tBu | H |

The compounds of formula

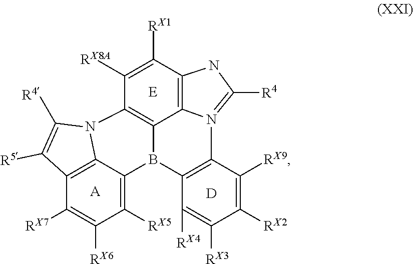

### Class 10

In the compounds of class 10 (formula (XXI)), the groups, residues and indices R⁴, R^{5′}, R^{4′}, R^{X1}, R^{X2}, R^{X3}, R^{X4}, R^{X5}, R^{X6}, R^{X7}, R^{X9} and R^{X8A} are defined above.

In the case that R^{X6} and R^{X7}, R^{X5} and R^{X6}, R^{X2} and R^{X3}, R^{X3} and R^{X4}, R^{X2} and R^{X9} and/or R^{X1} and R^{X8}, in the compounds of class 10 may form together an unsubstituted or substituted ring, the following rings (a) and (b) are formed:

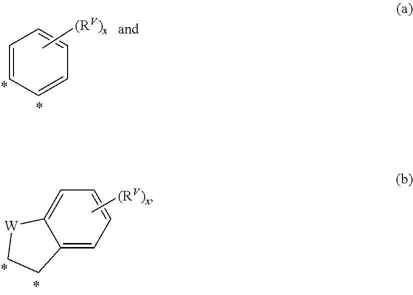

wherein

R^{V} represents H, a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group;

x represents 0, 1, 2, 3 or 4, preferably 0, 1 or 2, more preferably 0; and

W represents CR′″₂, O, S or NR^{IV};

R′″ represents a C₁-C₂₅ alkyl group, preferably a C₁-C₈ alkyl group, more preferably a C₁-C₄ alkyl group;

R^{IV} represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group; and

each * represents a point of attachment to ring A, D or E.

Most preferably, R⁴, R^{5′} and R^{4′} in the compounds of class 10 each independently represents methyl, ethyl, iso-propyl, sec-propyl, n-butyl, —C(Me)₂C₂H₅, tert-butyl, CF₃, SiPh₃, SiBuMe₂, unsubstituted phenyl, p-tert-butyl-phenyl, mesityl, xylyl, O-methyl-phenyl, or unsubstituted or substituted biphenyl, preferably unsubstituted biphenyl; or

R^{4′} and R^{5′} may form together an unsubstituted or substituted phenyl ring or an unsubstituted or substituted cyclohexene ring, preferably an unsubstituted phenyl ring or an unsubstituted cyclohexene ring.

Examples for compounds of class 10, wherein R^{4′} and R^{5′} may form together an unsubstituted phenyl ring or an unsubstituted cyclohexene ring are the following compounds:

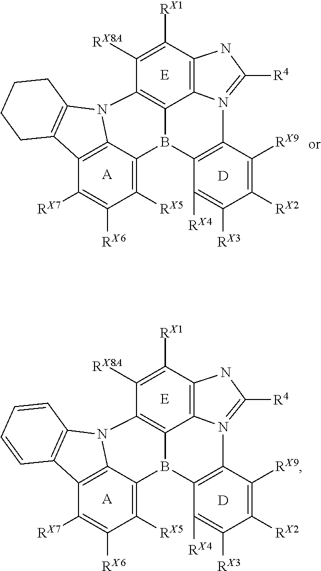

wherein the groups and residues are defined above and below.

Most preferably, R^{X5}, R^{X6}, R^{X7}, R⁹, R^{X2}, R^{X3} and R^{X4} in the compounds of class 10 each independently represents H, methyl, ethyl, iso-propyl, sec-propyl, n-butyl, tert-butyl, —C(Me)₂C₂H₅, unsubstituted phenyl, p-tert-butyl-phenyl, mesityl, xylyl, O-methyl-phenyl or unsubstituted or substituted biphenyl, preferably unsubstituted biphenyl.

Further most preferably, R^{X2}, R^{X5}, R^{X4}, R^{X7} and R^{X9} are H.

Most preferably, R^{X1} and R^{X8A} in the compounds of class 10 each independently represents H, methyl, ethyl, iso-propyl, sec-propyl, n-butyl, tert-butyl, —C(Me)₂C₂H₅, F, unsubstituted phenyl, mesityl, xylyl, —O-phenyl, —O-methyl, —NPh₂, —N-carbazolyl or —N(C₆H₅^{t}Bu)₂.

Further most preferably, R^{X8A} is H.

Examples for compounds of class 10 are compounds of the following formula (X), wherein the residues R⁴, R^{5′}, R^{4′}, R^{X1}, R^{X3} and R^{X6} are defined above.

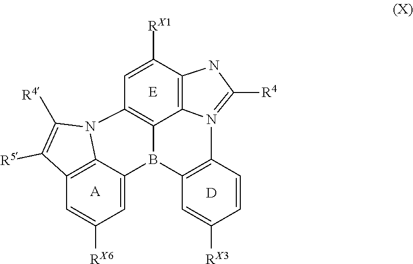

Preferred compounds (X) are mentioned in the following table, wherein Groups 1 and 10 defined as follows:

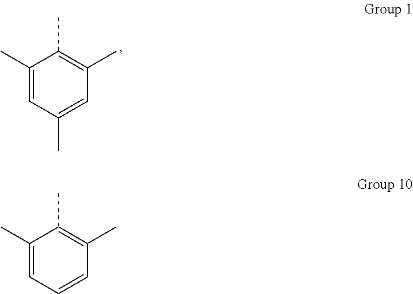

| R⁴ | R^{4′} | R^{5′} | R^{X3} | R^{X6} | R^{X1} |
|---|---|---|---|---|---|
| H | H | H | H | H | H |
| Ph | Ph | Ph | H | H | H |
| Me | Ph | Ph | H | H | H |
| Me | Ph | Group1 | H | H | H |
| Me | Ph | tBu | H | H | H |
| Me | Ph | SiPh3 | H | H | H |
| Me | Ph | Group10 | H | H | H |
| Me | Group1 | Group1 | H | H | H |
| Me | Group1 | tBu | H | H | H |
| Me | Group1 | SiPh3 | H | H | H |
| Me | Group1 | Group10 | H | H | H |
| Me | tBu | Group1 | H | H | H |
| Me | tBu | tBu | H | H | H |
| Me | tBu | SiPh3 | H | H | H |
| Me | tBu | Group10 | H | H | H |
| Ph | SiPh3 | Group1 | H | H | H |
| Ph | SiPh3 | tBu | H | H | H |
| Ph | SiPh3 | SiPh3 | H | H | H |
| Ph | SiPh3 | Group10 | H | H | H |
| Ph | Group10 | Group1 | H | H | H |
| Ph | Group10 | tBu | H | H | H |
| Ph | Group10 | SiPh3 | H | H | H |
| Ph | Group10 | Group10 | H | H | H |
| Ph | Ph | Ph | H | H | H |
| Ph | Ph | Group1 | H | H | H |
| Ph | Ph | tBu | H | H | H |
| Ph | Ph | SiPh3 | H | H | H |
| Ph | Ph | Group10 | H | H | H |
| Ph | Group1 | Group1 | H | H | H |
| Ph | Group1 | tBu | H | H | H |
| Ph | Group1 | SiPh3 | H | H | H |
| Ph | Group1 | Group10 | H | H | H |
| Ph | tBu | Group1 | H | H | H |
| Ph | tBu | tBu | H | H | H |
| Ph | tBu | SiPh3 | H | H | H |
| Ph | tBu | Group10 | H | H | H |
| Me | SiPh3 | Group1 | H | H | H |
| Me | SiPh3 | tBu | H | H | H |
| Me | SiPh3 | SiPh3 | H | H | H |
| Me | SiPh3 | Group10 | H | H | H |
| Me | Group10 | Group1 | H | H | H |
| Me | Group10 | tBu | H | H | H |
| Me | Group10 | SiPh3 | H | H | H |
| Me | Group10 | Group10 | H | H | H |
| Ph | Ph | Ph | H | H | tBu |
| Ph | Ph | Group1 | H | H | tBu |
| Ph | Ph | tBu | H | H | tBu |
| Ph | Ph | SiPh3 | H | H | tBu |
| Ph | Ph | Group10 | H | H | tBu |
| Ph | Group1 | Group1 | H | H | tBu |
| Ph | Group1 | tBu | H | H | tBu |
| Ph | Group1 | SiPh3 | H | H | tBu |
| Ph | Group1 | Group10 | H | H | tBu |
| Ph | tBu | Group1 | H | H | tBu |
| Ph | tBu | tBu | H | H | tBu |
| Ph | tBu | SiPh3 | H | H | tBu |
| Ph | tBu | Group10 | H | H | tBu |
| Me | SiPh3 | Group1 | H | H | tBu |
| Me | SiPh3 | tBu | H | H | tBu |
| Me | SiPh3 | SiPh3 | H | H | tBu |
| Me | SiPh3 | Group10 | H | H | tBu |
| Me | Group10 | Group1 | H | H | tBu |
| Me | Group10 | tBu | H | H | tBu |
| Me | Group10 | SiPh3 | H | H | tBu |
| Me | Group10 | Group10 | H | H | tBu |
| Ph | Ph | Ph | H | tBu | tBu |
| Ph | Ph | Group1 | H | tBu | tBu |
| Ph | Ph | tBu | H | tBu | tBu |
| Ph | Ph | SiPh3 | H | tBu | tBu |
| Ph | Ph | Group10 | H | tBu | tBu |
| Ph | Group1 | Group1 | H | tBu | tBu |
| Ph | Group1 | tBu | H | tBu | tBu |
| Ph | Group1 | SiPh3 | H | tBu | tBu |
| Ph | Group1 | Group10 | H | tBu | tBu |
| Ph | tBu | Group1 | H | tBu | tBu |
| Ph | tBu | tBu | H | tBu | tBu |
| Ph | tBu | SiPh3 | H | tBu | tBu |
| Ph | tBu | Group10 | H | tBu | tBu |
| Me | SiPh3 | Group1 | H | tBu | tBu |
| Me | SiPh3 | tBu | H | tBu | tBu |
| Me | SiPh3 | SiPh3 | H | tBu | tBu |
| Me | SiPh3 | Group10 | H | tBu | tBu |
| Me | Group10 | Group1 | H | tBu | tBu |
| Me | Group10 | tBu | H | tBu | tBu |
| Me | Group10 | SiPh3 | H | tBu | tBu |
| Me | Group10 | Group10 | H | tBu | tBu |
| Ph | Ph | Ph | Me | tBu | tBu |
| Ph | Ph | Group1 | Me | tBu | tBu |
| Ph | Ph | tBu | Me | tBu | tBu |
| Ph | Ph | SiPh3 | Me | tBu | tBu |
| Ph | Ph | Group10 | Me | tBu | tBu |
| Ph | Group1 | Group1 | Me | tBu | tBu |
| Ph | Group1 | tBu | Me | tBu | tBu |
| Ph | Group1 | SiPh3 | Me | tBu | tBu |
| Ph | Group1 | Group10 | Me | tBu | tBu |
| Ph | tBu | Group1 | Me | tBu | tBu |
| Ph | tBu | tBu | Me | tBu | tBu |
| Ph | tBu | SiPh3 | Me | tBu | tBu |
| Ph | tBu | Group10 | Me | tBu | tBu |
| Me | SiPh3 | Group1 | Me | tBu | tBu |
| Me | SiPh3 | tBu | Me | tBu | tBu |
| Me | SiPh3 | SiPh3 | Me | tBu | tBu |
| Me | SiPh3 | Group10 | Me | tBu | tBu |
| Me | Group10 | Group1 | Me | tBu | tBu |
| Me | Group10 | tBu | Me | tBu | tBu |
| Me | Group10 | SiPh3 | Me | tBu | tBu |
| Me | Group10 | Group10 | Me | tBu | tBu |

Further examples for compounds of class 10 are compounds of the following formula (Y), wherein the residues R⁴, R^{X1}, R^{X3} and R^{X6} are defined above.

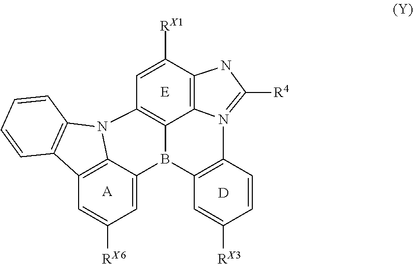

Preferred compounds (Y) are mentioned in the following table, wherein Groups 1 and 10 defined as follows:

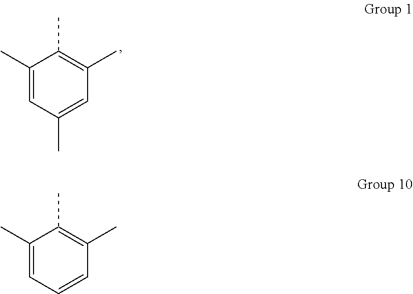

| R⁴ | R^{X3} | R^{X6} | R^{X1} |
|---|---|---|---|
| Me | H | H | H |
| Ph | H | H | H |
| group1 | H | H | H |
| group10 | H | H | H |
| SiPh3 | H | H | H |
| SiPh3 | H | H | H |
| Me | H | tBu | tBu |
| Ph | H | tBu | tBu |
| group1 | H | tBu | tBu |
| group10 | H | tBu | tBu |
| SiPh3 | H | tBu | tBu |
| SiPh3 | H | tBu | tBu |
| Me | Me | tBu | tBu |
| Ph | Me | tBu | tBu |

The compounds of formula

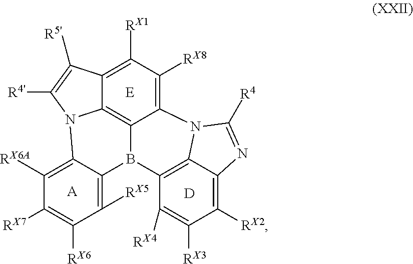

Class 11 In the compounds of class 11 (formula (XXII)), the groups, residues and indices R⁴, R^{5′}, R^{4′}, R^{X1}, R^{X2}, R^{X3}, R^{X4}, R^{X5}, R^{X6}, R^{X7}, R^{X8} and R^{X6A} are defined above.

In the case that R^{X6A} and R^{X7}, R^{X6} and R^{X7}, R^{X5} and R^{X6}, R^{X2} and R^{X3}, R^{X3} and R^{X4} and/or R^{X1} and R^{X8}, in the compounds of class 11 may form together an unsubstituted or substituted ring, the following rings (a) and (b) are formed:

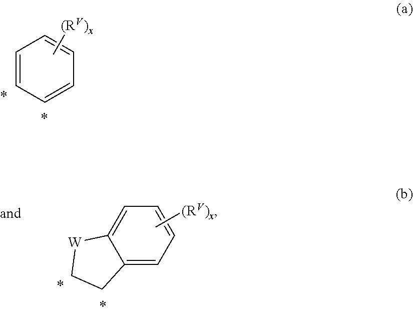

wherein

R^{V} represents H, a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group;

x represents 0, 1, 2, 3 or 4, preferably 0, 1 or 2, more preferably 0; and

W represents CR′″₂, O, S or NR^{IV};

R′″ represents a C₁-C₂₅ alkyl group, preferably a C₁-C₈ alkyl group, more preferably a C₁-C₄ alkyl group;

R^{IV} represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group; and

each * represents a point of attachment to ring A, D or E.

Most preferably, R⁴, R^{5′} and R^{4′} in the compounds of class 11 each independently represents methyl, ethyl, iso-propyl, sec-propyl, n-butyl, —C(Me)₂C₂H₅, tert-butyl, CF₃, SiPh₃, SiBuMe₂, unsubstituted phenyl, p-tert-butyl-phenyl, mesityl, xylyl, O-methyl-phenyl, or unsubstituted or substituted biphenyl, preferably unsubstituted biphenyl; or

R^{4′} and R^{5′} may form together an unsubstituted or substituted phenyl ring or an unsubstituted or substituted cyclohexene ring, preferably an unsubstituted phenyl ring or an unsubstituted cyclohexene ring.

Examples for compounds of class 11, wherein R^{4′} and R^{5′} may form together an unsubstituted phenyl ring or an unsubstituted cyclohexene ring are the following compounds:

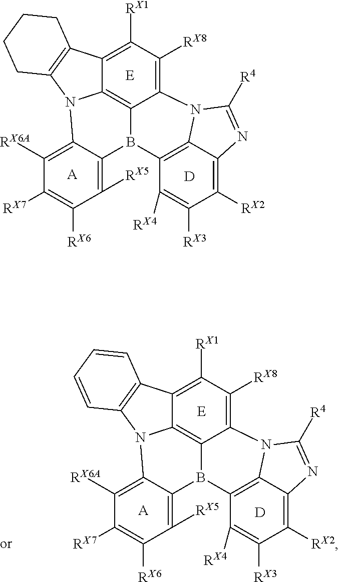

wherein the groups and residues are defined above and below.

Most preferably, R^{X5}, R^{X6}, R^{X6A} and R^{X7}, R^{X2}, R^{X3} and R^{X4} in the compounds of class 11 each independently represents H, methyl, ethyl, iso-propyl, sec-propyl, n-butyl, tert-butyl, —C(Me)₂C₂H₅, unsubstituted phenyl, p-tert-butyl-phenyl, mesityl, xylyl, O-methyl-phenyl or unsubstituted or substituted biphenyl, preferably unsubstituted biphenyl.

Most preferably, R^{X1} and R^{X8} in the compounds of class 11 each independently represents H, methyl, ethyl, iso-propyl, sec-propyl, n-butyl, tert-butyl, —C(Me)₂C₂H₅, F, unsubstituted phenyl, mesityl, xylyl, —O-phenyl, —O-methyl, —NPh₂, —N-carbazolyl, N-tert-butyl carbazol or —N(C₆H₅^{t}Bu)₂.

Further most preferably, R^{X8} is H.

Further most preferably, R^{X3}, R^{X4}, R^{X5}, R^{X6A} and R^{X7} are H.

Examples for compounds of class 11 are compounds of the following formula (Z1), wherein the residues R⁴, R^{5′}, R^{4′}, R^{X1}, R^{X2} and R^{X6} are defined above.

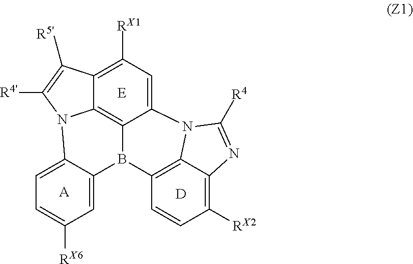

Preferred compounds (Z1) are mentioned in the following table, wherein Groups 1 and 10 defined as follows:

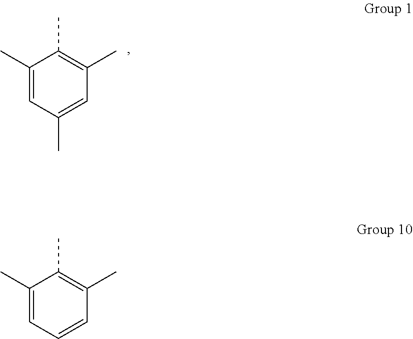

| R^{4′} | R⁴ | R^{5′} | R^{X2} | R^{X6} | R^{X1} |
|---|---|---|---|---|---|
| H | H | H | H | H | H |
| Ph | Ph | Ph | H | H | H |
| Me | Ph | Ph | H | H | H |
| Me | Ph | Group1 | H | H | H |
| Me | Ph | tBu | H | H | H |
| Me | Ph | SiPh3 | H | H | H |
| Me | Ph | Group10 | H | H | H |
| Me | Group1 | Group1 | H | H | H |
| Me | Group1 | tBu | H | H | H |
| Me | Group1 | SiPh3 | H | H | H |
| Me | Group1 | Group10 | H | H | H |
| Me | tBu | Group1 | H | H | H |
| Me | tBu | tBu | H | H | H |
| Me | tBu | SiPh3 | H | H | H |
| Me | tBu | Group10 | H | H | H |
| Ph | SiPh3 | Group1 | H | H | H |
| Ph | SiPh3 | tBu | H | H | H |
| Ph | SiPh3 | SiPh3 | H | H | H |
| Ph | SiPh3 | Group10 | H | H | H |
| Ph | Group10 | Group1 | H | H | H |
| Ph | Group10 | tBu | H | H | H |
| Ph | Group10 | SiPh3 | H | H | H |
| Ph | Group10 | Group10 | H | H | H |
| Ph | Ph | Ph | H | H | H |
| Ph | Ph | Group1 | H | H | H |
| Ph | Ph | tBu | H | H | H |
| Ph | Ph | SiPh3 | H | H | H |
| Ph | Ph | Group10 | H | H | H |
| Ph | Group1 | Group1 | H | H | H |
| Ph | Group1 | tBu | H | H | H |
| Ph | Group1 | SiPh3 | H | H | H |
| Ph | Group1 | Group10 | H | H | H |
| Ph | tBu | Group1 | H | H | H |
| Ph | tBu | tBu | H | H | H |
| Ph | tBu | SiPh3 | H | H | H |
| Ph | tBu | Group10 | H | H | H |
| Me | SiPh3 | Group1 | H | H | H |
| Me | SiPh3 | tBu | H | H | H |
| Me | SiPh3 | SiPh3 | H | H | H |
| Me | SiPh3 | Group10 | H | H | H |
| Me | Group10 | Group1 | H | H | H |
| Me | Group10 | tBu | H | H | H |
| Me | Group10 | SiPh3 | H | H | H |
| Me | Group10 | Group10 | H | H | H |
| Ph | Ph | Ph | tBu | H | H |
| Ph | Ph | Group1 | tBu | H | H |
| Ph | Ph | tBu | tBu | H | H |
| Ph | Ph | SiPh3 | tBu | H | H |
| Ph | Ph | Group10 | tBu | H | H |
| Ph | Group1 | Group1 | tBu | H | H |
| Ph | Group1 | tBu | tBu | H | H |
| Ph | Group1 | SiPh3 | tBu | H | H |
| Ph | Group1 | Group10 | tBu | H | H |
| Ph | tBu | Group1 | tBu | H | H |
| Ph | tBu | tBu | tBu | H | H |
| Ph | tBu | SiPh3 | tBu | H | H |
| Ph | tBu | Group10 | tBu | H | H |
| Me | SiPh3 | Group1 | tBu | H | H |
| Me | SiPh3 | tBu | tBu | H | H |
| Me | SiPh3 | SiPh3 | tBu | H | H |
| Me | SiPh3 | Group10 | tBu | H | H |
| Me | Group10 | Group1 | tBu | H | H |
| Me | Group10 | tBu | tBu | H | H |
| Me | Group10 | SiPh3 | tBu | H | H |
| Me | Group10 | Group10 | tBu | H | H |
| Ph | Ph | Ph | tBu | tBu | H |
| Ph | Ph | Group1 | tBu | tBu | H |
| Ph | Ph | tBu | tBu | tBu | H |
| Ph | Ph | SiPh3 | tBu | tBu | H |
| Ph | Ph | Group10 | tBu | tBu | H |
| Ph | Group1 | Group1 | tBu | tBu | H |
| Ph | Group1 | tBu | tBu | tBu | H |
| Ph | Group1 | SiPh3 | tBu | tBu | H |
| Ph | Group1 | Group10 | tBu | tBu | H |
| Ph | tBu | Group1 | tBu | tBu | H |
| Ph | tBu | tBu | tBu | tBu | H |
| Ph | tBu | SiPh3 | tBu | tBu | H |
| Ph | tBu | Group10 | tBu | tBu | H |
| Me | SiPh3 | Group1 | tBu | tBu | H |
| Me | SiPh3 | tBu | tBu | tBu | H |
| Me | SiPh3 | SiPh3 | tBu | tBu | H |
| Me | SiPh3 | Group10 | tBu | tBu | H |
| Me | Group10 | Group1 | tBu | tBu | H |
| Me | Group10 | tBu | tBu | tBu | H |
| Me | Group10 | SiPh3 | tBu | tBu | H |
| Me | Group10 | Group10 | tBu | tBu | H |
| Ph | Ph | Ph | tBu | tBu | Me |
| Ph | Ph | Group1 | tBu | tBu | Me |
| Ph | Ph | tBu | tBu | tBu | Me |
| Ph | Ph | SiPh3 | tBu | tBu | Me |
| Ph | Ph | Group10 | tBu | tBu | Me |
| Ph | Group1 | Group1 | tBu | tBu | Me |
| Ph | Group1 | tBu | tBu | tBu | Me |
| Ph | Group1 | SiPh3 | tBu | tBu | Me |
| Ph | Group1 | Group10 | tBu | tBu | Me |
| Ph | tBu | Group1 | tBu | tBu | Me |
| Ph | tBu | tBu | tBu | tBu | Me |
| Ph | tBu | SiPh3 | tBu | tBu | Me |
| Ph | tBu | Group10 | tBu | tBu | Me |
| Me | SiPh3 | Group1 | tBu | tBu | Me |
| Me | SiPh3 | tBu | tBu | tBu | Me |
| Me | SiPh3 | SiPh3 | tBu | tBu | Me |
| Me | SiPh3 | Group10 | tBu | tBu | Me |
| Me | Group10 | Group1 | tBu | tBu | Me |
| Me | Group10 | tBu | tBu | tBu | Me |
| Me | Group10 | SiPh3 | tBu | tBu | Me |
| Me | Group10 | Group10 | tBu | tBu | Me |

Further examples for compounds of class 11 are compounds of the following formula (Z2), wherein the residues R⁴, R^{X1}, R^{X2} and R^{X6} are defined above.

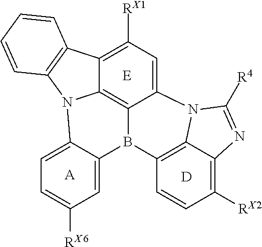

Preferred compounds (Z2) are mentioned in the following table, wherein Groups 1 and 10 defined as follows:

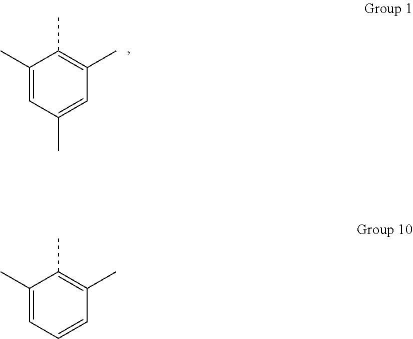

| R⁴ | R^{X2} | R^{X6} | R^{X1} |
|---|---|---|---|
| Me | H | H | H |
| Ph | H | H | H |
| group1 | H | H | H |
| group10 | H | H | H |
| SiPh3 | H | H | H |
| SiPh3 | H | H | H |
| Me | tBu | tBu | H |
| Ph | tBu | tBu | H |
| group1 | tBu | tBu | H |
| group10 | tBu | tBu | H |
| SiPh3 | tBu | tBu | H |
| SiPh3 | tBu | tBu | H |
| Me | tBu | tBu | Me |
| Ph | tBu | tBu | Me |

The compounds of formula

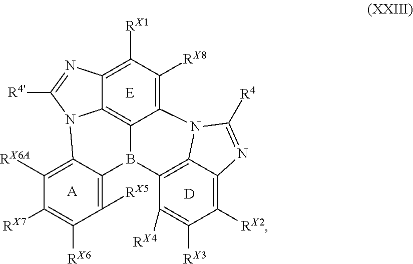

### Class 12

In the compounds of class 12 (formula (XXIII)), the groups, residues and indices R⁴, R^{4′}, R^{X1}, R^{X2}, R^{X3}, R^{X4}, R^{X5}, R^{X6}, R^{X7}, R^{X8} and R^{X6A} are defined above.

In the case that R^{X6A} and R^{X7}, R^{X6} and R^{X7}, R^{X5} and R^{X6}, R^{X2} and R^{X3}, R^{X3} and R^{X4} and/or R^{X1} and R^{X8}, in the compounds of class 12 may form together an unsubstituted or substituted ring, the following rings (a) and (b) are formed:

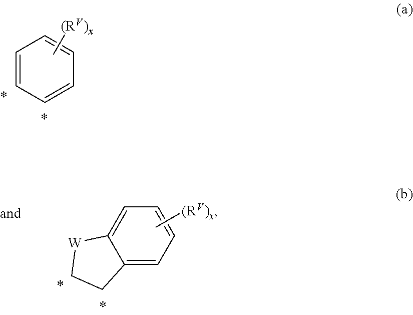

wherein

R^{V} represents H, a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group;

x represents 0, 1, 2, 3 or 4, preferably 0, 1 or 2, more preferably 0; and W represents CR′″₂, O, S or NR^{IV};

R′″ represents a C₁-C₂₅ alkyl group, preferably a C₁-C₈ alkyl group, more preferably a C₁-C₄ alkyl group;

R^{IV} represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group; and

each * represents a point of attachment to ring A, D or E.

Most preferably, R⁴ and R^{4′} in the compounds of class 12 each independently represents methyl, ethyl, iso-propyl, sec-propyl, n-butyl, —C(Me)₂C₂H₅, tert-butyl, CF₃, SiPh₃, SiBuMe₂, unsubstituted phenyl, p-tert-butyl-phenyl, mesityl, xylyl, O-methyl-phenyl, or unsubstituted or substituted biphenyl, preferably unsubstituted biphenyl.

Most preferably, R^{X5}, R^{X6}, R^{X6A} and R^{X7}, R^{X2}, R^{X3} and R^{X4} in the compounds of class 12 each independently represents H, methyl, ethyl, iso-propyl, sec-propyl, n-butyl, tert-butyl, —C(Me)₂C₂H₅, unsubstituted phenyl, p-tert-butyl-phenyl, mesityl, xylyl, O-methyl-phenyl or unsubstituted or substituted biphenyl, preferably unsubstituted biphenyl.

Most preferably, R^{X1} and R^{X8} in the compounds of class 12 each independently represents H, methyl, ethyl, iso-propyl, sec-propyl, n-butyl, tert-butyl, —C(Me)₂C₂H₅, F, unsubstituted phenyl, mesityl, xylyl, —O-phenyl, —O-methyl, —NPh₂, —N-carbazolyl, N-tert-butyl carbazol or —N(C₆H₅^{t}Bu)₂.

Further most preferably, R^{X8} is H.

Further most preferably, R^{X3}, R^{X4}, R^{X5}, R^{X6A} and R^{X7} are H.

Examples for compounds of class 12 are compounds of the following formula (Z3), wherein the residues R⁴, R^{4′}, R^{X1}, R^{X2} and R^{X6} are defined above.

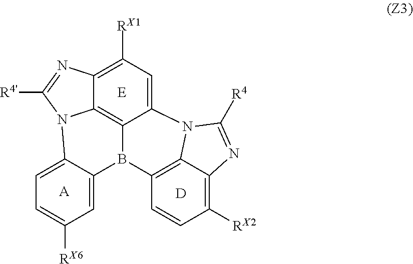

Preferred compounds (Z3) are mentioned in the following table, wherein Groups 1 and 10 defined as follows:

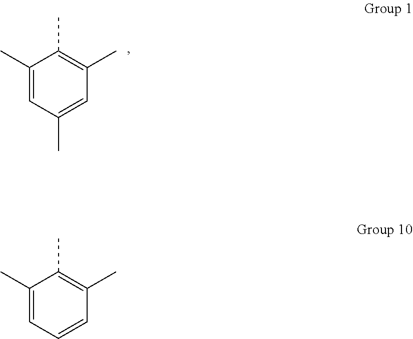

| R^{4′} | R⁴ | R^{X2} | R^{X6} | R^{X1} |
|---|---|---|---|---|
| H | H | H | H | H |
| Ph | Ph | H | H | H |
| Ph | Group1 | H | H | H |
| Ph | Group10 | H | H | H |
| Ph | SiPh3 | H | H | H |
| Ph | tBu | H | H | H |
| Group1 | Ph | H | H | H |
| Group1 | Group1 | H | H | H |
| Group1 | Group10 | H | H | H |
| Group1 | SiPh3 | H | H | H |
| Group1 | tBu | H | H | H |
| Group10 | Ph | H | H | H |
| Group10 | Group1 | H | H | H |
| Group10 | Group10 | H | H | H |
| Group10 | SiPh3 | H | H | H |
| Group10 | tBu | H | H | H |
| SiPh3 | Ph | H | H | H |
| SiPh3 | Group1 | H | H | H |
| SiPh3 | Group10 | H | H | H |
| SiPh3 | SiPh3 | H | H | H |
| SiPh3 | tBu | H | H | H |
| tBu | Ph | H | H | H |
| tBu | Group1 | H | H | H |
| tBu | Group10 | H | H | H |
| tBu | SiPh3 | H | H | H |
| tBu | tBu | H | H | H |
| H | H | tBu | H | H |
| Ph | Ph | tBu | H | H |
| Ph | Group1 | tBu | H | H |
| Ph | Group10 | tBu | H | H |
| Ph | SiPh3 | tBu | H | H |
| Ph | tBu | tBu | H | H |
| Group1 | Ph | tBu | H | H |
| Group1 | Group1 | tBu | H | H |
| Group1 | Group10 | tBu | H | H |
| Group1 | SiPh3 | tBu | H | H |
| Group1 | tBu | tBu | H | H |
| Group10 | Ph | tBu | H | H |
| Group10 | Group1 | tBu | H | H |
| Group10 | Group10 | tBu | H | H |
| Group10 | SiPh3 | tBu | H | H |
| Group10 | tBu | tBu | H | H |
| SiPh3 | Ph | tBu | H | H |
| SiPh3 | Group1 | tBu | H | H |
| SiPh3 | Group10 | tBu | H | H |
| SiPh3 | SiPh3 | tBu | H | H |
| SiPh3 | tBu | tBu | H | H |
| tBu | Ph | tBu | H | H |
| tBu | Group1 | tBu | H | H |
| tBu | Group10 | tBu | H | H |
| tBu | SiPh3 | tBu | H | H |
| tBu | tBu | tBu | H | H |
| H | H | Group1 | H | H |
| Ph | Ph | Group1 | H | H |
| Ph | Group1 | Group1 | H | H |
| Ph | Group10 | Group1 | H | H |
| Ph | SiPh3 | Group1 | H | H |
| Ph | tBu | Group1 | H | H |
| Group1 | Ph | Group1 | H | H |
| Group1 | Group1 | Group1 | H | H |
| Group1 | Group10 | Group1 | H | H |
| Group1 | SiPh3 | Group1 | H | H |
| Group1 | tBu | Group1 | H | H |
| Group10 | Ph | Group1 | H | H |
| Group10 | Group1 | Group1 | H | H |
| Group10 | Group10 | Group1 | H | H |
| Group10 | SiPh3 | Group1 | H | H |
| Group10 | tBu | Group1 | H | H |
| SiPh3 | Ph | Group1 | H | H |
| SiPh3 | Group1 | Group1 | H | H |
| SiPh3 | Group10 | Group1 | H | H |
| SiPh3 | SiPh3 | Group1 | H | H |
| SiPh3 | tBu | Group1 | H | H |
| tBu | Ph | Group1 | H | H |
| tBu | Group1 | Group1 | H | H |
| tBu | Group10 | Group1 | H | H |
| tBu | SiPh3 | Group1 | H | H |
| tBu | tBu | Group1 | H | H |
| H | H | Group1 | tBu | H |
| Ph | Group1 | Group1 | tBu | H |
| Ph | Group10 | Group1 | tBu | H |
| Ph | SiPh3 | Group1 | tBu | H |
| Ph | tBu | Group1 | tBu | H |
| Group1 | Ph | Group1 | tBu | H |
| Group1 | Group1 | Group1 | tBu | H |
| Group1 | Group10 | Group1 | tBu | H |
| Group1 | SiPh3 | Group1 | tBu | H |
| Group1 | tBu | Group1 | tBu | H |
| Group10 | Ph | Group1 | tBu | H |
| Group10 | Group1 | Group1 | tBu | H |
| Group10 | Group10 | Group1 | tBu | H |
| Group10 | SiPh3 | Group1 | tBu | H |
| Group10 | tBu | Group1 | tBu | H |
| SiPh3 | Ph | Group1 | tBu | H |
| SiPh3 | Group1 | Group1 | tBu | H |
| SiPh3 | Group10 | Group1 | tBu | H |
| SiPh3 | SiPh3 | Group1 | tBu | H |
| SiPh3 | tBu | Group1 | tBu | H |
| tBu | Ph | Group1 | tBu | H |
| tBu | Group1 | Group1 | tBu | H |
| tBu | Group10 | Group1 | tBu | H |
| tBu | SiPh3 | Group1 | tBu | H |
| tBu | tBu | Group1 | tBu | H |
| H | H | Group1 | tBu | Me |
| Ph | Ph | Group1 | tBu | Me |
| Ph | Group1 | Group1 | tBu | Me |
| Ph | Group10 | Group1 | tBu | Me |
| Ph | SiPh3 | Group1 | tBu | Me |
| Ph | tBu | Group1 | tBu | Me |
| Group1 | Ph | Group1 | tBu | Me |
| Group1 | Group1 | Group1 | tBu | Me |
| Group1 | Group10 | Group1 | tBu | Me |
| Group1 | SiPh3 | Group1 | tBu | Me |
| Group1 | tBu | Group1 | tBu | Me |
| Group10 | Ph | Group1 | tBu | Me |
| Group10 | Group1 | Group1 | tBu | Me |
| Group10 | Group10 | Group1 | tBu | Me |
| Group10 | SiPh3 | Group1 | tBu | Me |
| Group10 | tBu | Group1 | tBu | Me |
| SiPh3 | Ph | Group1 | tBu | Me |
| SiPh3 | Group1 | Group1 | tBu | Me |
| SiPh3 | Group10 | Group1 | tBu | Me |
| SiPh3 | SiPh3 | Group1 | tBu | Me |
| SiPh3 | tBu | Group1 | tBu | Me |
| tBu | Ph | Group1 | tBu | Me |
| tBu | Group1 | Group1 | tBu | Me |
| tBu | Group10 | Group1 | tBu | Me |
| tBu | SiPh3 | Group1 | tBu | Me |
| tBu | tBu | Group1 | tBu | Me |

The compounds of formula

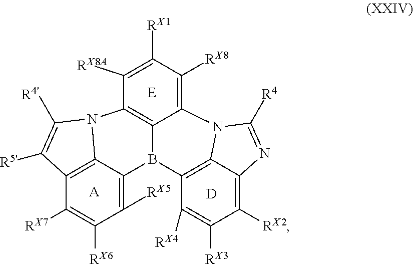

### Class 13

In the compounds of class 13 (formula (XXIV)), the groups, residues and indices R⁴, R^{4′}, R^{5′}, R^{X1}, R^{X2}, R^{X3}, R^{X4}, R^{X5}, R^{X6}, R^{X7}, R^{X8} and R^{X8A} are defined above.

In the case that R^{X6} and R^{X7}, R^{X5} and R^{X6}, R^{X2} and R^{X3}, R^{X3} and R^{X4}, R^{X8A} and R^{X1} and/or R^{X1} and R^{X8}, in the compounds of class 13 may form together an unsubstituted or substituted ring, the following rings (a) and (b) are formed:

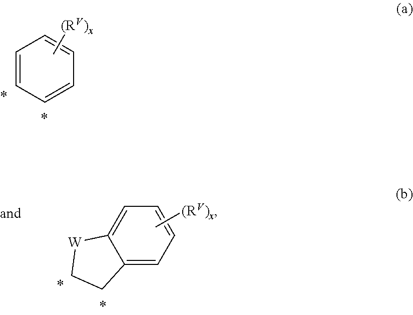

wherein

R^{V} represents H, a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group;

x represents 0, 1, 2, 3 or 4, preferably 0, 1 or 2, more preferably 0; and

W represents CR′″₂, O, S or NR^{IV};

R′″ represents a C₁-C₂₅ alkyl group, preferably a C₁-C₈ alkyl group, more preferably a C₁-C₄ alkyl group;

R^{IV} represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group; and

each * represents a point of attachment to ring A, D or E.

Most preferably, R⁴, R^{4′} and R^{5′} in the compounds of class 13 each independently represents methyl, ethyl, iso-propyl, sec-propyl, n-butyl, tert.-butyl, —C(Me)₂C₂H₅, CF₃, SiPh₃, SiBuMe₂, unsubstituted phenyl, p-tert-butyl-phenyl, mesityl, xylyl, O-methyl-phenyl, m-(tert-butyl)₂-phenyl or unsubstituted or substituted biphenyl, preferably unsubstituted biphenyl; or R^{4′} and R^{5′} may form together an unsubstituted or substituted phenyl ring or an unsubstituted or substituted cyclohexene ring, preferably an unsubstituted phenyl ring or an unsubstituted cyclohexene ring.

Examples for compounds of class 13, wherein R^{4′} and R^{5′} may form together an unsubstituted phenyl ring or an unsubstituted cyclohexene ring are the following compounds:

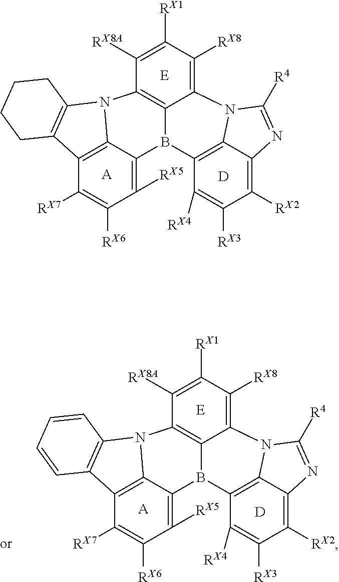

wherein the groups and residues are defined above and below.

Most preferably, R^{X5}, R^{X6} and R^{X7} and R^{X2}, R^{X3} and R^{X4} in the compounds of class 13 each independently represents H, methyl, ethyl, iso-propyl, sec-propyl, n-butyl, tert-butyl, —C(Me)₂C₂H₅, F, unsubstituted phenyl, p-tert-butyl-phenyl, mesityl, xylyl, O-methyl-phenyl or unsubstituted or substituted biphenyl, preferably unsubstituted biphenyl.

Most preferably, R^{X1}, R^{X8} and R^{X8A} in the compounds of class 13 each independently represents H, methyl, ethyl, iso-propyl, sec-propyl, n-butyl, tert-butyl, —C(Me)₂C₂H₅, F, unsubstituted phenyl, —O-phenyl, —O-methyl, —NPh₂, —N-carbazolyl, N-tert-butyl-carbazolyl or —N(C₆H₅^{t}Bu)₂.

Most preferably, R^{X4} and R^{X5} in the compounds of class 13 are H, and further most preferably R^{X4}, R^{X5}, R^{X7}, R^{X8} and R^{X8A} are H.

Examples for compounds of class 13 are compounds of the following formula (Z⁴), wherein the residues R⁴, R^{4′}, R^{5′}, R^{X1}, R^{X2}, R^{X3} and R^{X6} are defined above.

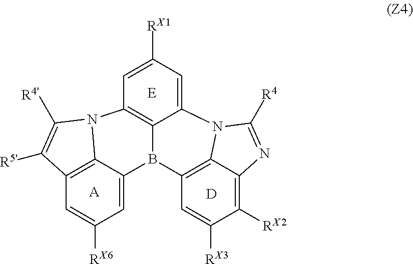

Preferred compounds (Z⁴) are mentioned in the following table, wherein Groups 1 and 10 defined as follows:

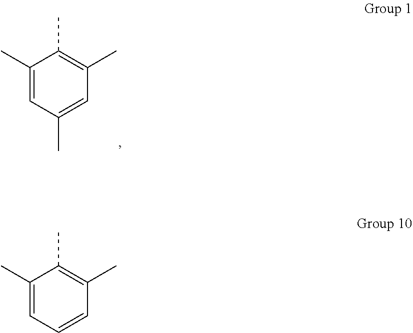

| R^{4′} | R⁴ | R^{5′} | R^{X6} | R^{X1} | R^{X2} | R^{X3} |
|---|---|---|---|---|---|---|
| Ph | Ph | Ph | H | H | H | H |
| Me | Me | Me | H | H | H | H |
| Me | Ph | Ph | H | H | H | H |
| Me | Ph | Group1 | H | H | H | H |
| Me | Ph | Group10 | H | H | H | H |
| Me | Ph | tBu | H | H | H | H |
| Me | Ph | SiPh3 | H | H | H | H |
| Ph | Ph | Ph | H | H | H | H |
| Ph | Ph | Group1 | H | H | H | H |
| Ph | Ph | Group10 | H | H | H | H |
| Ph | Ph | tBu | H | H | H | H |
| Ph | Ph | SiPh3 | H | H | H | H |
| Me | Group1 | Ph | H | H | H | H |
| Me | Group1 | Group1 | H | H | H | H |
| Me | Group1 | Group10 | H | H | H | H |
| Me | Group1 | tBu | H | H | H | H |
| Me | Group1 | SiPh3 | H | H | H | H |
| Ph | Group1 | Ph | H | H | H | H |
| Ph | Group1 | Group1 | H | H | H | H |
| Ph | Group1 | Group10 | H | H | H | H |
| Ph | Group1 | tBu | H | H | H | H |
| Ph | Group1 | SiPh3 | H | H | H | H |
| Ph | Group10 | Ph | H | H | H | H |
| Ph | Group10 | Group1 | H | H | H | H |
| Ph | Group10 | Group10 | H | H | H | H |
| Ph | Group10 | tBu | H | H | H | H |
| Ph | Group10 | SiPh3 | H | H | H | H |
| Me | Group10 | Ph | H | H | H | H |
| Me | Group10 | Group1 | H | H | H | H |
| Me | Group10 | Group10 | H | H | H | H |
| Me | Group10 | tBu | H | H | H | H |
| Me | Group10 | SiPh3 | H | H | H | H |
| Ph | Ph | Ph | tBu | H | H | H |
| Me | Me | Me | tBu | H | H | H |
| Me | Ph | Ph | tBu | H | H | H |
| Me | Ph | Group1 | tBu | H | H | H |
| Me | Ph | Group10 | tBu | H | H | H |
| Me | Ph | tBu | tBu | H | H | H |
| Me | Ph | SiPh3 | tBu | H | H | H |
| Ph | Ph | Ph | tBu | H | H | H |
| Ph | Ph | Group1 | tBu | H | H | H |
| Ph | Ph | Group10 | tBu | H | H | H |
| Ph | Ph | tBu | tBu | H | H | H |
| Ph | Ph | SiPh3 | tBu | H | H | H |
| Me | Group1 | Ph | tBu | H | H | H |
| Me | Group1 | Group1 | tBu | H | H | H |
| Me | Group1 | Group10 | tBu | H | H | H |
| Me | Group1 | tBu | tBu | H | H | H |
| Me | Group1 | SiPh3 | tBu | H | H | H |
| Ph | Group1 | Ph | tBu | H | H | H |
| Ph | Group1 | Group1 | tBu | H | H | H |
| Ph | Group1 | Group10 | tBu | H | H | H |
| Ph | Group1 | tBu | tBu | H | H | H |
| Ph | Group1 | SiPh3 | tBu | H | H | H |
| Ph | Group10 | Ph | tBu | H | H | H |
| Ph | Group10 | Group1 | tBu | H | H | H |
| Ph | Group10 | Group10 | tBu | H | H | H |
| Ph | Group10 | tBu | tBu | H | H | H |
| Ph | Group10 | SiPh3 | tBu | H | H | H |
| Me | Group10 | Ph | tBu | H | H | H |
| Me | Group10 | Group1 | tBu | H | H | H |
| Me | Group10 | Group10 | tBu | H | H | H |
| Me | Group10 | tBu | tBu | H | H | H |
| Me | Group10 | SiPh3 | tBu | H | H | H |
| Ph | Ph | Ph | tBu | H | Group1 | H |
| Me | Me | Me | tBu | H | Group1 | H |
| Me | Ph | Ph | tBu | H | Group1 | H |
| Me | Ph | Group1 | tBu | H | Group1 | H |
| Me | Ph | Group10 | tBu | H | Group1 | H |
| Me | Ph | tBu | tBu | H | Group1 | H |
| Me | Ph | SiPh3 | tBu | H | Group1 | H |
| Ph | Ph | Ph | tBu | H | Group1 | H |
| Ph | Ph | Group1 | tBu | H | Group1 | H |
| Ph | Ph | Group10 | tBu | H | Group1 | H |
| Ph | Ph | tBu | tBu | H | Group1 | H |
| Ph | Ph | SiPh3 | tBu | H | Group1 | H |
| Me | Group1 | Ph | tBu | H | Group1 | H |
| Me | Group1 | Group1 | tBu | H | Group1 | H |
| Me | Group1 | Group10 | tBu | H | Group1 | H |
| Me | Group1 | tBu | tBu | H | Group1 | H |
| Me | Group1 | SiPh3 | tBu | H | Group1 | H |
| Ph | Group1 | Ph | tBu | H | Group1 | H |
| Ph | Group1 | Group1 | tBu | H | Group1 | H |
| Ph | Group1 | Group10 | tBu | H | Group1 | H |
| Ph | Group1 | tBu | tBu | H | Group1 | H |
| Ph | Group1 | SiPh3 | tBu | H | Group1 | H |
| Ph | Group10 | Ph | tBu | H | Group1 | H |
| Ph | Group10 | Group1 | tBu | H | Group1 | H |
| Ph | Group10 | Group10 | tBu | H | Group1 | H |
| Ph | Group10 | tBu | tBu | H | Group1 | H |
| Ph | Group10 | SiPh3 | tBu | H | Group1 | H |
| Me | Group10 | Ph | tBu | H | Group1 | H |
| Me | Group10 | Group1 | tBu | H | Group1 | H |
| Me | Group10 | Group10 | tBu | H | Group1 | H |
| Me | Group10 | tBu | tBu | H | Group1 | H |
| Me | Group10 | SiPh3 | tBu | H | Group1 | H |
| Ph | Ph | Ph | tBu | H | Group10 | H |
| Me | Me | Me | tBu | H | Group10 | H |
| Me | Ph | Ph | tBu | H | Group10 | H |
| Me | Ph | Group1 | tBu | H | Group10 | H |
| Me | Ph | Group10 | tBu | H | Group10 | H |
| Me | Ph | tBu | tBu | H | Group10 | H |
| Me | Ph | SiPh3 | tBu | H | Group10 | H |
| Ph | Ph | Ph | tBu | H | Group10 | H |
| Ph | Ph | Group1 | tBu | H | Group10 | H |
| Ph | Ph | Group10 | tBu | H | Group10 | H |
| Ph | Ph | tBu | tBu | H | Group10 | H |
| Ph | Ph | SiPh3 | tBu | H | Group10 | H |
| Me | Group1 | Ph | tBu | H | Group10 | H |
| Me | Group1 | Group1 | tBu | H | Group10 | H |
| Me | Group1 | Group10 | tBu | H | Group10 | H |
| Me | Group1 | tBu | tBu | H | Group10 | H |
| Me | Group1 | SiPh3 | tBu | H | Group10 | H |
| Ph | Group1 | Ph | tBu | H | Group10 | H |
| Ph | Group1 | Group1 | tBu | H | Group10 | H |
| Ph | Group1 | Group10 | tBu | H | Group10 | H |
| Ph | Group1 | tBu | tBu | H | Group10 | H |
| Ph | Group1 | SiPh3 | tBu | H | Group10 | H |
| Ph | Group10 | Ph | tBu | H | Group10 | H |
| Ph | Group10 | Group1 | tBu | H | Group10 | H |
| Ph | Group10 | Group10 | tBu | H | Group10 | H |
| Ph | Group10 | tBu | tBu | H | Group10 | H |
| Ph | Group10 | SiPh3 | tBu | H | Group10 | H |
| Me | Group10 | Ph | tBu | H | Group10 | H |
| Me | Group10 | Group1 | tBu | H | Group10 | H |
| Me | Group10 | Group10 | tBu | H | Group10 | H |
| Me | Group10 | tBu | tBu | H | Group10 | H |
| Me | Group10 | SiPh3 | tBu | H | Group10 | H |
| Ph | Ph | Ph | tBu | H | H | tBu |
| Me | Me | Me | tBu | H | H | tBu |
| Me | Ph | Ph | tBu | H | H | tBu |
| Me | Ph | Group1 | tBu | H | H | tBu |
| Me | Ph | Group10 | tBu | H | H | tBu |
| Me | Ph | tBu | tBu | H | H | tBu |
| Me | Ph | SiPh3 | tBu | H | H | tBu |
| Ph | Ph | Ph | tBu | H | H | tBu |
| Ph | Ph | Group1 | tBu | H | H | tBu |
| Ph | Ph | Group10 | tBu | H | H | tBu |
| Ph | Ph | tBu | tBu | H | H | tBu |
| Ph | Ph | SiPh3 | tBu | H | H | tBu |
| Me | Group1 | Ph | tBu | H | H | tBu |
| Me | Group1 | Group1 | tBu | H | H | tBu |
| Me | Group1 | Group10 | tBu | H | H | tBu |
| Me | Group1 | tBu | tBu | H | H | tBu |
| Me | Group1 | SiPh3 | tBu | H | H | tBu |
| Ph | Group1 | Ph | tBu | H | H | tBu |
| Ph | Group1 | Group1 | tBu | H | H | tBu |
| Ph | Group1 | Group10 | tBu | H | H | tBu |
| Ph | Group1 | tBu | tBu | H | H | tBu |
| Ph | Group1 | SiPh3 | tBu | H | H | tBu |
| Ph | Group10 | Ph | tBu | H | H | tBu |
| Ph | Group10 | Group1 | tBu | H | H | tBu |
| Ph | Group10 | Group10 | tBu | H | H | tBu |
| Ph | Group10 | tBu | tBu | H | H | tBu |
| Ph | Group10 | SiPh3 | tBu | H | H | tBu |
| Me | Group10 | Ph | tBu | H | H | tBu |
| Me | Group10 | Group1 | tBu | H | H | tBu |
| Me | Group10 | Group10 | tBu | H | H | tBu |
| Me | Group10 | tBu | tBu | H | H | tBu |
| Me | Group10 | SiPh3 | tBu | H | H | tBu |
| Ph | Ph | Ph | tBu | Me | H | tBu |
| Me | Me | Me | tBu | Me | H | tBu |
| Me | Ph | Ph | tBu | Me | H | tBu |
| Me | Ph | Group1 | tBu | Me | H | tBu |
| Me | Ph | Group10 | tBu | Me | H | tBu |
| Me | Ph | tBu | tBu | Me | H | tBu |
| Me | Ph | SiPh3 | tBu | Me | H | tBu |
| Ph | Ph | Ph | tBu | Me | H | tBu |
| Ph | Ph | Group1 | tBu | Me | H | tBu |
| Ph | Ph | Group10 | tBu | Me | H | tBu |
| Ph | Ph | tBu | tBu | Me | H | tBu |
| Ph | Ph | SiPh3 | tBu | Me | H | tBu |
| Me | Group1 | Ph | tBu | Me | H | tBu |
| Me | Group1 | Group1 | tBu | Me | H | tBu |
| Me | Group1 | Group10 | tBu | Me | H | tBu |
| Me | Group1 | tBu | tBu | Me | H | tBu |
| Me | Group1 | SiPh3 | tBu | Me | H | tBu |
| Ph | Group1 | Ph | tBu | Me | H | tBu |
| Ph | Group1 | Group1 | tBu | Me | H | tBu |
| Ph | Group1 | Group10 | tBu | Me | H | tBu |
| Ph | Group1 | tBu | tBu | Me | H | tBu |
| Ph | Group1 | SiPh3 | tBu | Me | H | tBu |
| Ph | Group10 | Ph | tBu | Me | H | tBu |
| Ph | Group10 | Group1 | tBu | Me | H | tBu |
| Ph | Group10 | Group10 | tBu | Me | H | tBu |
| Ph | Group10 | tBu | tBu | Me | H | tBu |
| Ph | Group10 | SiPh3 | tBu | Me | H | tBu |
| Me | Group10 | Ph | tBu | Me | H | tBu |
| Me | Group10 | Group1 | tBu | Me | H | tBu |
| Me | Group10 | Group10 | tBu | Me | H | tBu |
| Me | Group10 | tBu | tBu | Me | H | tBu |
| Me | Group10 | SiPh3 | tBu | Me | H | tBu |

The compounds of formula

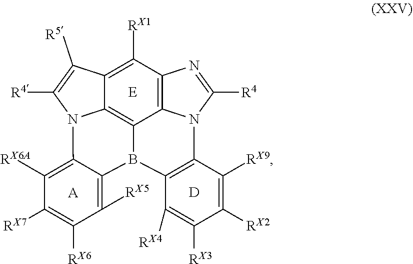

### Class 14

In the compounds of class 14 (formula (XXV)), the groups, residues and indices R⁴, R^{4′}, R^{5′}, R^{X1}, R^{X2}, R^{X3}, R^{X4}, R^{X5}, R^{X6}, R^{X7}, R^{X6A} and R^{X9} are defined above.

In the case that R^{X6A} and R^{X7}, R^{X6} and R^{X7}, R^{X5} and R^{X6}, R^{X2} and R^{X3}, R^{X3} and R^{X4} and/or R^{X2} and R^{X9}, in the compounds of class 14 may form together an unsubstituted or substituted ring, the following rings (a) and (b) are formed:

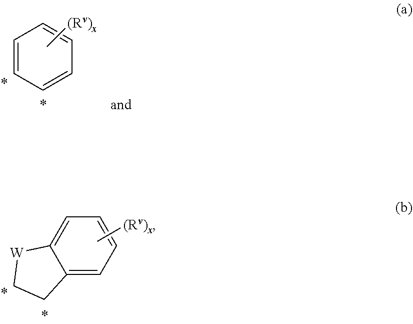

wherein

R^{V} represents H, a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group;

x represents 0, 1, 2, 3 or 4, preferably 0, 1 or 2, more preferably 0; and

W represents CR′″₂, O, S or NR^{IV};

R′″ represents a C₁-C₂₅ alkyl group, preferably a C₁-C₈ alkyl group, more preferably a C₁-C₄ alkyl group;

R^{IV} represents a C₁-C₂₅ alkyl group, preferably a C₁-C₈ alkyl group, more preferably a C₁-C₄ alkyl group, or an unsubstituted or substituted C₆-C₃₀ aryl group, preferably unsubstituted or substituted C₆-C₁₀ aryl group, more preferably unsubstituted or substituted C₆ aryl group, most preferably unsubstituted C₆ aryl group; and

each * represents a point of attachment to ring A or D.

Most preferably, R⁴, R^{4′} and R^{5′} in the compounds of class 14 each independently represents methyl, ethyl, iso-propyl, sec-propyl, n-butyl, tert.-butyl, —C(Me)₂C₂H₅, CF₃, SiPh₃, SiBuMe₂, unsubstituted phenyl, p-tert-butyl-phenyl, mesityl, xylyl, O-methyl-phenyl, m-(tert-butyl)₂-phenyl or unsubstituted or substituted biphenyl, preferably unsubstituted biphenyl; or

R^{4′} and R^{5′} may form together an unsubstituted or substituted phenyl ring or an unsubstituted or substituted cyclohexene ring, preferably an unsubstituted phenyl ring or an unsubstituted cyclohexene ring.

Examples for compounds of class 14, wherein R^{4′} and R^{5′} may form together an unsubstituted phenyl ring or an unsubstituted cyclohexene ring are the following compounds:

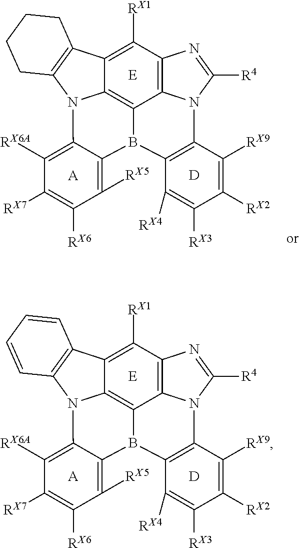

wherein the groups and residues are defined above and below.

Most preferably, R^{X5}, R^{X6}, R^{X6A} and R^{X7} and R^{X9}, R^{X2}, R^{X3} and R^{X4} in the compounds of class 14 each independently represents H, methyl, ethyl, iso-propyl, sec-propyl, n-butyl, tert-butyl, —C(Me)₂C₂H₅, F, unsubstituted phenyl, p-tert-butyl-phenyl, mesityl, xylyl, O-methyl-phenyl or unsubstituted or substituted biphenyl, preferably unsubstituted biphenyl.

Most preferably, R^{X1} in the compounds of class 14 represents H, methyl, ethyl, iso-propyl, sec-propyl, n-butyl, tert-butyl, —C(Me)₂C₂H₅, F, unsubstituted phenyl, —O-phenyl, —O-methyl, —NPh₂, SiPh₃, —N-carbazolyl, N-tert-butyl-carbazolyl or —N(C₆H₅^{t}Bu)₂.

Most preferably, R^{X4} and R^{X5} in the compounds of class 14 are H, and further most preferably R^{X4}, R^{X5}, R^{X6A} and R^{X9} are H.

Examples for compounds of class 14 are compounds of the following formula (Z5), wherein the residues R⁴, R^{4′}, R^{5′}, R^{X1}, R^{X2}, R^{X3} and R^{X6} are defined above.

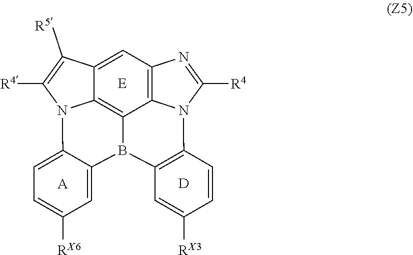

Preferred compounds (Z5) are mentioned in the following table, wherein Groups 1 and 10 defined as follows:

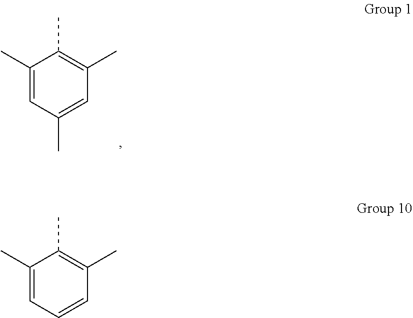

| R⁴ | R^{4′} | R^{5′} | R^{X6} | R^{X3} |
|---|---|---|---|---|
| Ph | Ph | Ph | H | H |
| Ph | Me | Group1 | H | H |
| Ph | Me | Group10 | H | H |
| Ph | Me | SiPh3 | H | H |
| Ph | Me | tBu | H | H |
| Ph | Ph | Ph | H | H |
| Ph | Ph | Group1 | H | H |
| Ph | Ph | Group10 | H | H |
| Ph | Ph | SiPh3 | H | H |
| Ph | Ph | tBu | H | H |
| Group1 | Me | Ph | H | H |
| Group1 | Me | Group1 | H | H |
| Group1 | Me | Group10 | H | H |
| Group1 | Me | SiPh3 | H | H |
| Group1 | Me | tBu | H | H |
| Group1 | Ph | Ph | H | H |
| Group1 | Ph | Group1 | H | H |
| Group1 | Ph | Group10 | H | H |
| Group1 | Ph | SiPh3 | H | H |
| Group1 | Ph | tBu | H | H |
| Group10 | Ph | Ph | H | H |
| Group10 | Ph | Group1 | H | H |
| Group10 | Ph | Group10 | H | H |
| Group10 | Ph | SiPh3 | H | H |
| Group10 | Ph | tBu | H | H |
| Group10 | Me | Ph | H | H |
| Group10 | Me | Group1 | H | H |
| Group10 | Me | Group10 | H | H |
| Group10 | Me | SiPh3 | H | H |
| Group10 | Me | tBu | H | H |
| tBu | Ph | Ph | H | H |
| tBu | Ph | Group1 | H | H |
| tBu | Ph | Group10 | H | H |
| tBu | Ph | SiPh3 | H | H |
| tBu | Ph | tBu | H | H |
| tBu | Me | Ph | H | H |
| tBu | Me | Group1 | H | H |
| tBu | Me | Group10 | H | H |
| tBu | Me | SiPh3 | H | H |
| tBu | Me | tBu | H | H |
| Ph | Ph | Ph | tBu | tBu |
| Ph | Me | Group1 | tBu | tBu |
| Ph | Me | Group10 | tBu | tBu |
| Ph | Me | SiPh3 | tBu | tBu |
| Ph | Me | tBu | tBu | tBu |
| Ph | Ph | Ph | tBu | tBu |
| Ph | Ph | Group1 | tBu | tBu |
| Ph | Ph | Group10 | tBu | tBu |
| Ph | Ph | SiPh3 | tBu | tBu |
| Ph | Ph | tBu | tBu | tBu |
| Group1 | Me | Ph | tBu | tBu |
| Group1 | Me | Group1 | tBu | tBu |
| Group1 | Me | Group10 | tBu | tBu |
| Group1 | Me | SiPh3 | tBu | tBu |
| Group1 | Me | tBu | tBu | tBu |
| Group1 | Ph | Ph | tBu | tBu |
| Group1 | Ph | Group1 | tBu | tBu |
| Group1 | Ph | Group10 | tBu | tBu |
| Group1 | Ph | SiPh3 | tBu | tBu |
| Group1 | Ph | tBu | tBu | tBu |
| Group10 | Ph | Ph | tBu | tBu |
| Group10 | Ph | Group1 | tBu | tBu |
| Group10 | Ph | Group10 | tBu | tBu |
| Group10 | Ph | SiPh3 | tBu | tBu |
| Group10 | Ph | tBu | tBu | tBu |
| Group10 | Me | Ph | tBu | tBu |
| Group10 | Me | Group1 | tBu | tBu |
| Group10 | Me | Group10 | tBu | tBu |
| Group10 | Me | SiPh3 | tBu | tBu |
| Group10 | Me | tBu | tBu | tBu |
| tBu | Ph | Ph | tBu | tBu |
| tBu | Ph | Group1 | tBu | tBu |
| tBu | Ph | Group10 | tBu | tBu |
| tBu | Ph | SiPh3 | tBu | tBu |
| tBu | Ph | tBu | tBu | tBu |
| tBu | Me | Ph | tBu | tBu |
| tBu | Me | Group1 | tBu | tBu |
| tBu | Me | Group10 | tBu | tBu |
| tBu | Me | SiPh3 | tBu | tBu |
| tBu | Me | tBu | tBu | tBu |

Preparation of the Compounds of Formula (I)

The compound represented by formula (I) can be synthesized in accordance with the reactions conducted in the Examples of the present application, and by using alternative reactions or raw materials suited to an intended product, in analogy to reactions and raw materials known in the art.

Examples for suitable preparation processes are mentioned below.

In one embodiment of the present invention, the compounds of formula (I) are prepared by a process comprising the step:

Borylation of a compound of formula (XXVI):

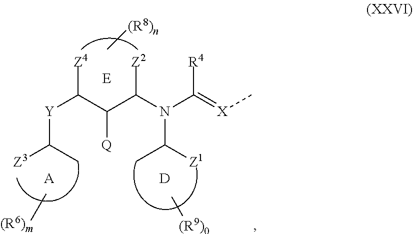

wherein

Q is halogen, or SiR¹⁴₃, preferably, Q is halogen, more preferably Cl or Br; and

R¹⁴ represents a substituted or unsubstituted alkyl group having 1 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, a substituted or unsubstituted aryloxy group having 6 to 30 ring carbon atoms;

and all other residues, symbols and indices mentioned in formula (XXVI) are defined above.

The compounds of formula (I) are according to one embodiment prepared by a process comprising the following step (i):

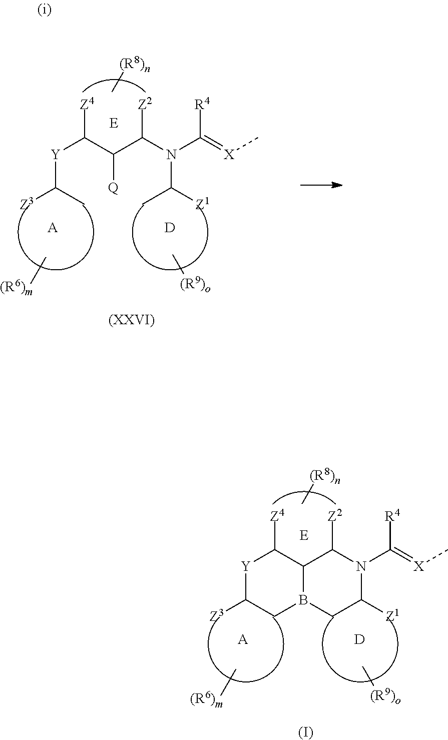

Reaction of a compound of formula (XXVI) with an alkyl lithium reagent, for example tert-butyl lithium, or sec-butyl lithium, or n-butyl lithium, in an organic solvent, for example tert-butyl benzene, xylene, or toluene, followed by reaction with a boron comprising Lewis acid like boron tribromide, boron trichloride, boron triiodide or boron trifluoride, preferably in the presence of an amine base, for example N-ethyl-N-isopropylpropan-2-amine, triethylamine, 2,6-lutidine, pyridine, 2,2,6,6-tetramethyl piperidine, or 2,4,6-tri-tert-butylpyridine. Suitable reaction conditions are mentioned in the examples.

According to a further embodiment, the compounds of formula (I) are prepared by a process comprising the following steps (ia) and (iia):

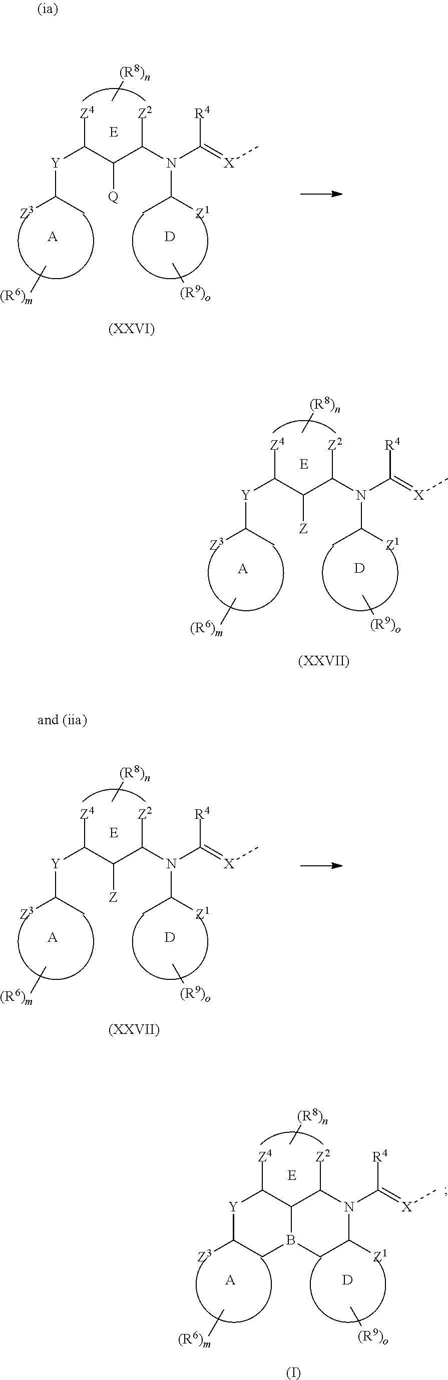

wherein

Z represents a B containing compound selected from the group consisting of B(R¹⁵)₂ and B(hal)₃⁻M⁺,

wherein

hal represents a halogen atom, preferably F;

M represents an alkali metal, preferably Na or K, and

R¹⁵ represents halogen, preferably F, Cl or Br, or OR¹⁶,

R¹⁶ represents H, an unsubstituted or substituted C₁ to C₁₈ alkyl group, preferably, methyl, ethyl, iso-propyl, or

two groups R¹⁶ may form together a ring, preferably a 6 or 5 membered ring, whereby preferably one of the following groups is formed

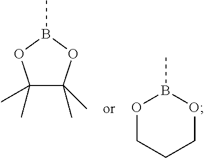

and all other residues, symbols and indices mentioned in formulae (XXVII) and (I) are defined above.

Step (ia):

Reaction of a compound of formula (XXVI) with an alkyl lithium reagent, for example tert-butyl lithium, sec-butyl lithium or n-butyl lithium, in an organic solvent, for example tert-butyl benzene, xylene, toluene, THF, dioxane, Et₂O, Bu₂O or MeOCH₂CH₂OMe, followed by reaction with a boron comprising compound, for example 4,4,5,5-tetramethyl-2-(1-methylethoxy)-1,3,2-dioxaborolane, whereby compound (XXVII) is obtained. Suitable reaction conditions are mentioned in the examples.

Step (iia)

Reaction of compound (XXVII) in an organic solvent, for example tert-butyl benzene, xylene, toluene, THF, dioxane, Et₂O, Bu₂O, MeOCH₂CH₂OMe, with a Lewis acid, for example BBr₃, BCl₃, BI₃, AlCl₃, AlBr₃, TiCl₄, ZrCl₄ or BF₃, preferably in the presence of an amine base, for example N-ethyl-N-isopropylpropan-2-amine, triethylamine, 2,6-lutidine, pyridine, 2,2,6,6-tetramethyl piperidine or 2,4,6-tri-tert-butylpyridine. Suitable reaction conditions are mentioned in the examples.

According to a further embodiment, the compounds of formula (I) are prepared by a process comprising the following steps (ib) and (iib):

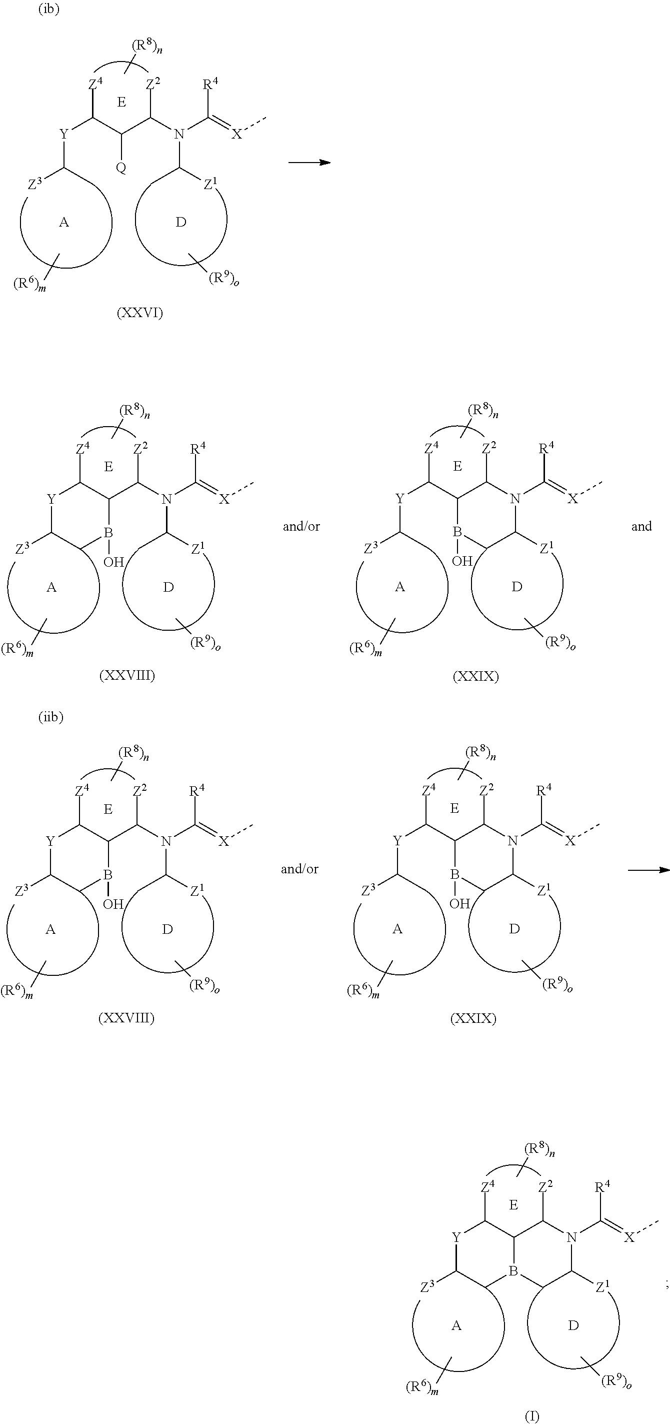

Step (ib)

Reaction of a compound of formula (XXVI) with an alkyl lithium reagent, for example tert-butyl lithium, sec-butyl lithium or n-butyl lithium, in an organic solvent, for example tert-butyl benzene, xylene, toluene, followed by reaction with a boron comprising Lewis acid, for example BBr₃, BCl₃, BI₃, or BF₃, preferably in the presence of an amine base, for example N-ethyl-N-isopropylpropan-2-amine, triethylamine, 2,6-lutidine, pyridine, 2,2,6,6-tetramethyl piperidine or 2,4,6-tri-tert-butylpyridine, whereby a compound of formula (XXVIII) and/or (XXIX) is obtained. Suitable reaction conditions are mentioned in the examples.

Step (iib)

Reaction of compound (XXVIII) and/or (XXIX) in an organic solvent, for example tert-butyl benzene, xylene, toluene, THF, dioxane, Et₂O, Bu₂O or MeOCH₂CH₂OMe, with a Lewis acid, for example, BBr₃, BCl₃, BI₃, AlCl₃, AlBr₃, TiCl₄, ZrCl₄ or BF₃, preferably in the presence of an amine base, for example N-ethyl-N-isopropylpropan-2-amine, triethylamine, 2,6-lutidine, pyridine, 2,2,6,6-tetramethyl piperidine or 2,4,6-tri-tert-butylpyridine. Suitable reaction conditions are mentioned in the examples.

According to a further embodiment, the compounds of formula (I) are prepared by a process comprising the following steps (ic), (iic) and (iiic):

ic) Transforming a compound of formula (XXVI) into a compound of formula (XXVII)

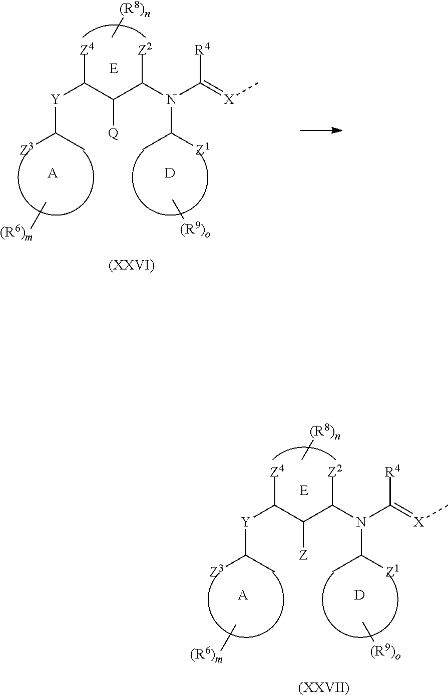

iic) Transforming a compound of formula (XXVII) into a compound of formula (XXVIII) and/or (XXIX)

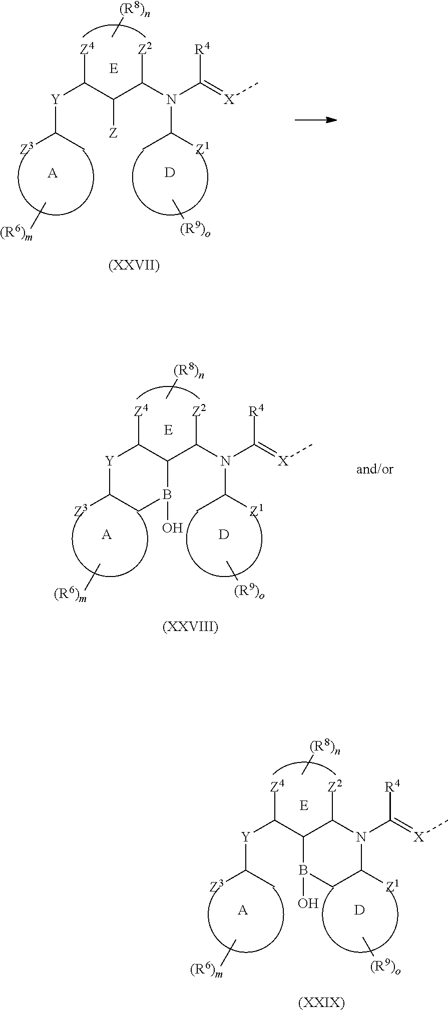

and

iiic) Transforming the compound of formula (XXVIII) and/or (XXIX) to a compound of formula (I)

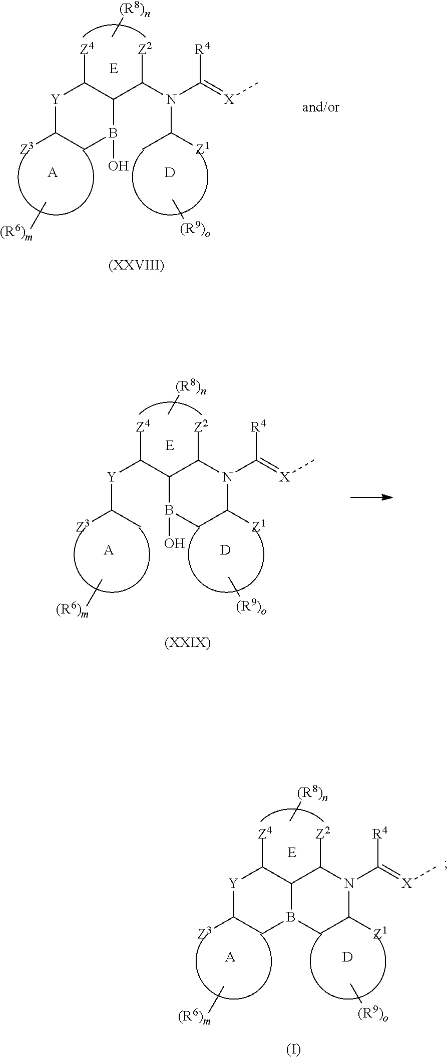

wherein the residues symbols and indices are mentioned above.

Step ic)

Reaction of a compound of formula (XXVI) with an alkyl lithium reagent, for example tert-butyl lithium, sec-butyl lithium or n-butyl lithium, in an organic solvent, for example tert-butyl benzene, xylene, toluene, THF, dioxane, Et₂O, Bu₂O or MeOCH₂CH₂OMe, followed by reaction with a boron comprising compound, for example 4,4,5,5-tetramethyl-2-(1-methylethoxy)-1,3,2-dioxaborolane, whereby compound (XXVII) is obtained. Suitable reaction conditions are mentioned in the examples.

Step iic)/iiic)

Reaction of compound (XXVII) in an organic solvent, for example tert-butyl benzene, xylene, toluene, THF, dioxane, Et₂O, Bu₂O or MeOCH₂CH₂OMe, with a Lewis acid, for example BBr₃, BCl₃, BI₃, AlCl₃, AlBr₃, TiCl₄, ZrCl₄ or BF₃, preferably in the presence of an amine base, for example N-ethyl-N-isopropylpropan-2-amine, triethylamine, 2,6-lutidine, pyridine, 2,2,6,6-tetramethyl piperidine or 2,4,6-tri-tert-butylpyridine via compound (XXVIII) and/or (XXIX) to compound (I). Suitable reaction conditions are mentioned in the examples.

A further subject of the present invention is a compound of formula (XXVI)

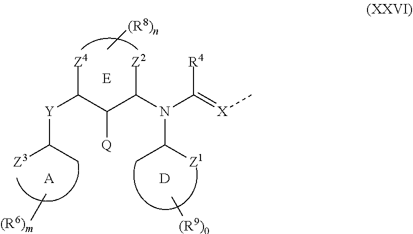

wherein

Q is halogen, or SiR¹⁴₃, preferably, Q is halogen, more preferably Cl or Br; and

R¹⁴ represents a substituted or unsubstituted alkyl group having 1 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, a substituted or unsubstituted aryloxy group having 6 to 30 ring carbon atoms;

and all other residues, symbols and indices mentioned in formula (XXVI) are defined above.

A further subject of the present invention is a compound of formula (X)XVII)

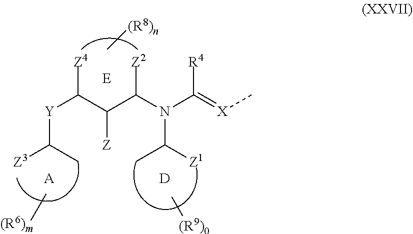

wherein

Z represents a B containing compound selected from the group consisting of B(R¹⁵)₂ and B(hal)₃⁻M⁺,

wherein

hal represents a halogen atom, preferably F;

M represents an alkali metal, preferably Na or K, and

R¹⁵ represents halogen, preferably F, Cl or Br, or OR¹⁶,

R¹⁶ represents H, an unsubstituted or substituted C₁ to C₁₈ alkyl group, preferably, methyl, ethyl, iso-propyl, or

two groups R¹⁶ may form together a ring, preferably a 6 or 5 membered ring, whereby preferably one of the following groups is formed

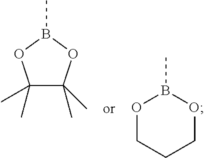

and all other residues, symbols and indices mentioned in formula (XXVII) are defined above.

A further subject of the present invention is a compound of formula (XXVIII) or (XXIX)

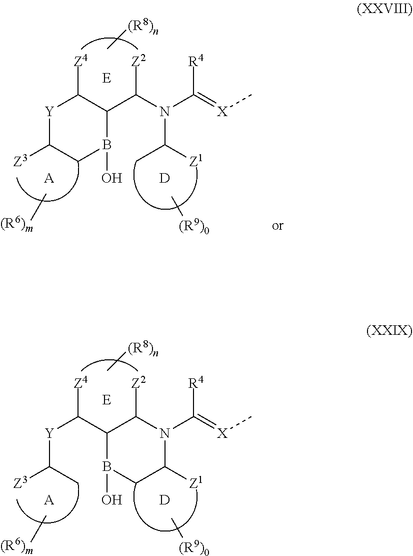

wherein and all residues, symbols and indices mentioned in formulae (XXVIII) and (XXIX) are defined above.

Organic Electroluminescence Device

According to one aspect of the present invention, a material for an organic electroluminescence device, comprising at least one compound of formula (I) is provided.

According to another aspect of the invention, the following organic electroluminescence device is provided: An organic electroluminescence device comprising a cathode, an anode, and one or more organic thin film layers comprising an emitting layer disposed between the cathode and the anode, wherein at least one layer of the organic thin film layers comprises at least one compound of formula (I).

According to another aspect of the invention, the use of a compound of formula (I) according to the present invention in an organic electroluminescence device is provided.

In the present specification, regarding “one or more organic thin film layers disposed between the cathode and the anode”, if only one organic layer is present between the cathode and the anode, it means the layer, and if plural organic layers are present between the cathode and the anode, it means at least one layer thereof.

In one embodiment, the organic EL device has a hole-transporting layer between the anode and the emitting layer.

In one embodiment, the organic EL device has an electron-transporting layer between the cathode and the emitting layer.

In the present specification, regarding the “one or more organic thin film layers between the emitting layer and the anode”, if only one organic layer is present between the emitting layer and the anode, it means that layer, and if plural organic layers are present, it means at least one layer thereof. For example, if two or more organic layers are present between the emitting layer and the anode, an organic layer nearer to the emitting layer is called the “hole-transporting layer”, and an organic layer nearer to the anode is called the “hole-injecting layer”. Each of the “hole-transporting layer” and the “hole-injecting layer” may be a single layer or may be formed of two or more layers. One of these layers may be a single layer and the other may be formed of two or more layers.

Similarly, regarding the “one or more organic thin film layers between the emitting layer and the cathode”, if only one organic layer is present between the emitting layer and the cathode, it means that layer, and if plural organic layers are present, it means at least one layer thereof. For example, if two or more organic layers are present between the emitting layer and the cathode, an organic layer nearer to the emitting layer is called the “electron-transporting layer”, and an organic layer nearer to the cathode is called the “electron-injecting layer”. Each of the “electron-transporting layer” and the “electron-injecting layer” may be a single layer or may be formed of two or more layers. One of these layers may be a single layer and the other may be formed of two or more layers.

The “one or more organic thin film layers comprising an emitting layer” mentioned above, preferably the emitting layer, comprises a compound represented by formula (I). The compound represented by formula (I) preferably functions as an emitting material, more preferably as a fluorescent emitting material, most preferably as a blue fluorescent emitting material. By the presence of a compound of formula (I) in the organic EL device, preferably in the emitting layer, the luminous efficiency of the organic EL device can be enhanced.

According to another aspect of the invention, an emitting layer of the organic electroluminescence device is provided which comprises least one compound of formula (I).

Preferably, the emitting layer comprises at least one emitting material (dopant material) and at least one host material, wherein the emitting material is at least one compound of formula (I).

Preferred host materials are substituted or unsubstituted polyaromatic hydrocarbon (PAH) compounds, substituted or unsubstituted polyheteroaromatic compounds, substituted or unsubstituted anthracene compounds, or substituted or unsubstituted pyrene compounds.

More preferably, the organic electroluminescence device according to the present invention comprises in the emitting layer least one compound of formula (I) as a dopant material and at least one host material selected from the group consisting of substituted or unsubstituted polyaromatic hydrocarbon (PAH) compounds, substituted or unsubstituted polyheteroaromatic compounds, substituted or unsubstituted anthracene compounds, and substituted or unsubstituted pyrene compounds. Preferably, the at least one host is at least one substituted or unsubstituted anthracene compound.

According to another aspect of the invention, an emitting layer of the organic electroluminescence device is provided which comprises least one compound of formula (I) as a dopant material and an anthracene compound as a host material.

Suitable anthracene compounds are represented by the following formula (10):

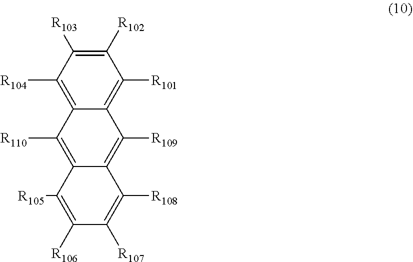

wherein

one or more pairs of two or more adjacent R₁₀₁ to R₁₁₀ may form a substituted or unsubstituted, saturated or unsaturated ring;

R₁₀₁ to R₁₁₀ that do not form the substituted or unsubstituted, saturated or unsaturated ring are independently a hydrogen atom, a substituted or unsubstituted alkyl group including 1 to 50 carbon atoms, a substituted or unsubstituted haloalkyl group including 1 to 50 carbon atoms, a substituted or unsubstituted alkenyl group including 2 to 50 carbon atoms, a substituted or unsubstituted alkynyl group including 2 to 50 carbon atoms, a substituted or unsubstituted cycloalkyl group including 3 to 50 ring carbon atoms, a substituted or unsubstituted alkoxy group including 1 to 50 carbon atoms, a substituted or unsubstituted alkylene group including 1 to 50 carbon atoms, a substituted or unsubstituted aryloxy group including 6 to 50 ring carbon atoms, a substituted or unsubstituted arylthio group including 6 to 50 ring carbon atoms, a substituted or unsubstituted aralkyl group including 7 to 50 carbon atoms, —Si(R₁₂₁)(R₁₂₂)(R₁₂₃), —C(═O)R₁₂₄, —COOR₁₂₅, —N(R₁₂₆)(R₁₂₇), a halogen atom, a cyano group, a nitro group, a substituted or unsubstituted aryl group including 6 to 50 ring carbon atoms, a substituted or unsubstituted monovalent heterocyclic group including 5 to 50 ring atoms, or a group represented by the following formula (31);

R₁₂₁ to R₁₂₇ are independently a hydrogen atom, a substituted or unsubstituted alkyl group including 1 to 50 carbon atoms, a substituted or unsubstituted cycloalkyl group including 3 to 50 ring carbon atoms, a substituted or unsubstituted aryl group including 6 to 50 ring carbon atoms or a substituted or unsubstituted monovalent heterocyclic group including 5 to 50 ring atoms; when each of R₁₂₁ to R₁₂₇ is present in plural, each of the plural R₁₂₁ to R₁₂₇ may be the same or different;

provided that at least one of R₁₀₁ to R₁₁₀ that do not form the substituted or unsubstituted, saturated or unsaturated ring is a group represented by the following formula (31). If two or more groups represented by the formula (31) are present, each of these groups may be the same or different;

-L₁₀₁-Ar₁₀₁ (31)

wherein in the formula (31),

L₁₀₁ is a single bond, a substituted or unsubstituted arylene group including 6 to 30 ring carbon atoms or a substituted or unsubstituted divalent heterocyclic group including 5 to 30 ring atoms;

Ar₁₀₁ is a substituted or unsubstituted aryl group including 6 to 50 ring carbon atoms or a substituted or unsubstituted monovalent heterocyclic group including 5 to 50 ring atoms.

Specific examples of each substituent, substituents for “substituted or unsubstituted” and the halogen atom in the compound (10) are the same as those mentioned above.

An explanation will be given on “one or more pairs of two or more adjacent R₁₀₁ to R₁₁₀ may form a substituted or unsubstituted, saturated or unsaturated ring”.

The “one pair of two or more adjacent R₁₀₁ to R₁₁₀” is a combination of R₁₀₁ and R₁₀₂, R₁₀₂ and R₁₀₃, R₁₀₃ and R₁₀₄, R₁₀₅ and R₁₀₆, R₁₀₆ and R₁₀₇, R₁₀₇ and R₁₀₈, R₁₀₈ and R₁₀₉, R₁₀₁ and R₁₀₂ and R₁₀₃ or the like, for example.

The substituent in “substituted” in the “substituted or unsubstituted” for the saturated or unsaturated ring is the same as those for “substituted or unsubstituted” mentioned in the formula (10).

The “saturated or unsaturated ring” means, when R₁₀₁ and R₁₀₂ form a ring, for example, a ring formed by a carbon atom with which R₁₀₁ is bonded, a carbon atom with which R₁₀₂ is bonded and one or more arbitrary elements. Specifically, when a ring is formed by R₁₀₁ and R₁₀₂, when an unsaturated ring is formed by a carbon atom with which R₁₀₁ is bonded, a carbon atom with R₁₀₂ is bonded and four carbon atoms, the ring formed by R₁₀₁ and R₁₀₂ is a benzene ring.

The “arbitrary element” is preferably a C element, a N element, an O element or a S element.

In the arbitrary element (C element or N element, for example), atomic bondings that do not form a ring may be terminated by a hydrogen atom, or the like.

The “one or more arbitrary element” is preferably 2 or more and 15 or less, more preferably 3 or more and 12 or less, and further preferably 3 or more and 5 or less arbitrary elements.

For example, R₁₀₁ and R₁₀₂ may form a ring, and simultaneously, R₁₀₅ and R₁₀₆ may form a ring. In this case, the compound represented by the formula (10) is a compound represented by the following formula (10A), for example:

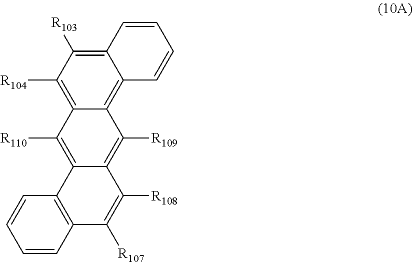

In one embodiment, R₁₀₁ to R₁₁₀ are independently a hydrogen atom, a substituted or unsubstituted alkyl group including 1 to 50 carbon atoms, a substituted or unsubstituted aryl group including 6 to 50 ring carbon atoms, a substituted or unsubstituted heterocyclic group including 5 to 50 ring atoms or a group represented by the formula (31).

Preferably, R₁₀₁ to R₁₁₀ are independently a hydrogen atom, a substituted or unsubstituted aryl group including 6 to 50 ring carbon atoms, a substituted or unsubstituted heterocyclic group including 5 to 50 ring atoms or a group represented by the formula (31).

More preferably, R₁₀₁ to R₁₁₀ are independently a hydrogen atom, a substituted or unsubstituted aryl group including 6 to 18 ring carbon atoms, a substituted or unsubstituted heterocyclic group including 5 to 18 ring atoms or a group represented by the formula (31).

Most preferably, at least one of R₁₀₉ and R₁₁₀ is a group represented by the formula (31).

Further most preferably, R₁₀₉ and R₁₁₀ are independently a group represented by the formula (31).

In one embodiment, the compound (10) is a compound represented by the following formula (10-1)

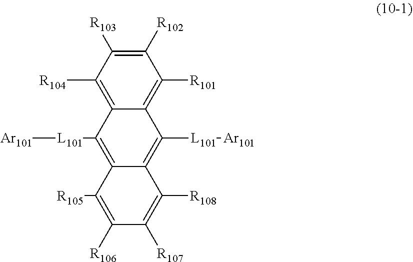

wherein in the formula (10-1), R₁₀₁ to R₁₀₃, L₁₀₁ and Ar₁₀₁ are as defined in the formula (10).

In one embodiment, the compound (10) is a compound represented by the following formula (10-2):

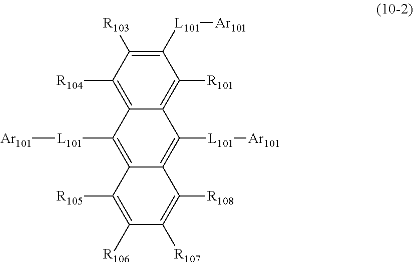

wherein in the formula (10-2), R₁₀₁, R₁₀₃ to R₁₀₈, L₁₀₁ and Ar₁₀₁ are as defined in the formula (10).

In one embodiment, the compound (10) is a compound represented by the following formula (10-3):

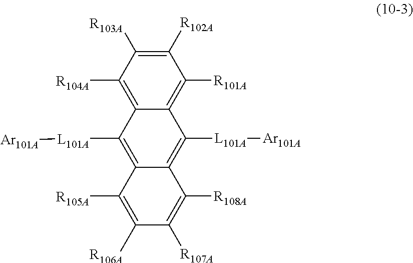

wherein in the formula (10-3),

R_{101A} to R_{108A} are independently a hydrogen atom or a substituted or unsubstituted aryl group including 6 to 50 ring carbon atoms;

L_{101A} is a single bond or a substituted or unsubstituted arylene group including 6 to 30 ring carbon atoms, and the two L_{101A}s may be the same or different;

Ar_{101A} is a substituted or unsubstituted aryl group including 6 to 50 ring carbon atoms, and the two Ar_{101A}s may be the same or different.

In one embodiment, the compound (10) is a compound represented by the following formula (10-4):

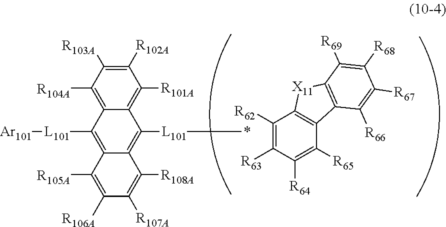

wherein in the formula (10-4),

L₁₀₁ and Ar₁₀₁ are as defined in the formula (10);

R_{101A} to R_{108A} are independently a hydrogen atom or a substituted or unsubstituted aryl group including 6 to 50 ring carbon atoms;

X₁₁ is O, S, or N(R₆₁);

R₆₁ is a hydrogen atom, a substituted or unsubstituted alkyl group including 1 to 50 carbon atoms or a substituted or unsubstituted aryl group including 6 to 50 ring carbon atoms: one of R₆₂ to R₆₉ is an atomic bonding that is bonded with L₁₀₁;

one or more pairs of adjacent R₆₂ to R₆₉ that are not bonded with L₁₀₁ may be bonded with each other to form a substituted or unsubstituted, saturated or unsaturated ring; and

R₆₂ to R₆₉ that are not bonded with L₁₀₁ and do not form the substituted or unsubstituted, saturated or unsaturated ring are independently a hydrogen atom, a substituted or unsubstituted alkyl group including 1 to 50 carbon atoms or a substituted or unsubstituted aryl group including 6 to 50 ring carbon atoms.

In one embodiment, the compound (10) is a compound represented by the following formula (10-4A):

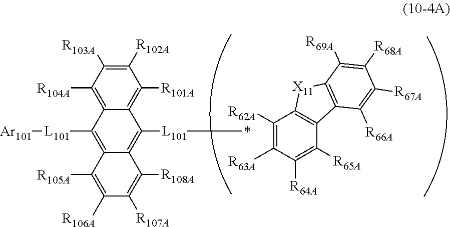

wherein in the formula (10-4A),

L₁₀₁ and Ar₁₀₁ are as defined in the formula (10); R_{101A} to R_{108A} are independently a hydrogen atom or a substituted or unsubstituted aryl group including 6 to 50 ring carbon atoms;

X₁₁ is O, S or N(R₆₁);

R₆₁ is a hydrogen atom, a substituted or unsubstituted alkyl group including 1 to 50 carbon atoms or a substituted or unsubstituted aryl group including 6 to 50 ring carbon atoms; one or more pairs of adjacent two or more of R_{62A} to R_{69A} may form a substituted or unsubstituted, saturated or unsaturated ring, and adjacent two of R_{62A} to R_{69A} form a ring represented by the following formula (10-4A-1); and

R_{62A} to R_{69A} that do not form a substituted or unsubstituted, saturated or unsaturated ring are independently a hydrogen atom, a substituted or unsubstituted alkyl group including 1 to 50 carbon atoms or a substituted or unsubstituted aryl group including 6 to 50 ring carbon atoms.

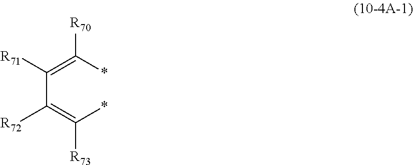

wherein in the formula (10-4A-1),

each of the two atomic bondings * is bonded with adjacent two of R_{62A} to R_{69A};

one of R₇₀ to R₇₃ is an atomic bonding that is bonded with L₁₀₁; and

R₇₀ to R₇₃ that are not bonded with L₁₀₁ are independently a hydrogen atom, a substituted or unsubstituted alkyl group including 1 to 50 carbon atoms or a substituted or unsubstituted aryl group including 6 to 50 ring carbon atoms.

In one embodiment, the compound (10) is a compound represented by the following formula (10-6):

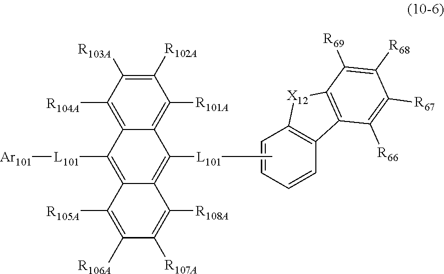

wherein in the formula (10-6),

L₁₀₁ and Ar₁₀₁ are as defined in the formula (10);

R_{101A} to R_{108A} are as defined in the formula (10-4);

R₆₆ to R₆₉ are as defined in the formula (10-4); and

X₁₂ is O or S.

In one embodiment, the compound represented by the formula (10-6) is a compound represented by the following formula (10-6H):

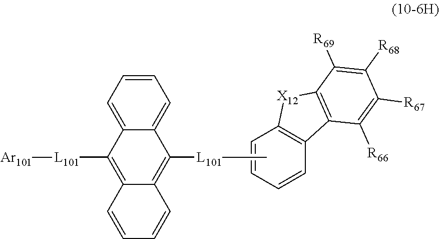

wherein in the formula (10-6H),

L₁₀₁ and Ar₁₀₁ are as defined in the formula (10);

R₆₆ to R₆₉ are as defined in the formula (10-4); and

X₁₂ is O or S.

In one embodiment, the compound represented by the formulas (10-6) and (10-6H) is a compound represented by the following formula (10-6Ha):

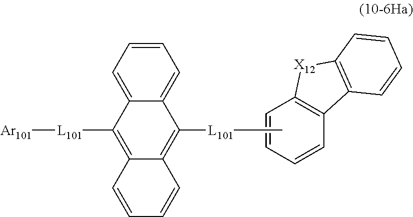

wherein in the formula (10-6Ha),

L₁₀₁ and Ar₁₀₁ are as defined in the formula (10); and

X₁₂ is O or S.

In one embodiment, the compound represented by the formulas (10-6), (10-6H) and (10-6Ha) is a compound represented by the following formula (10-6Ha-1) or (10-6Ha-2):

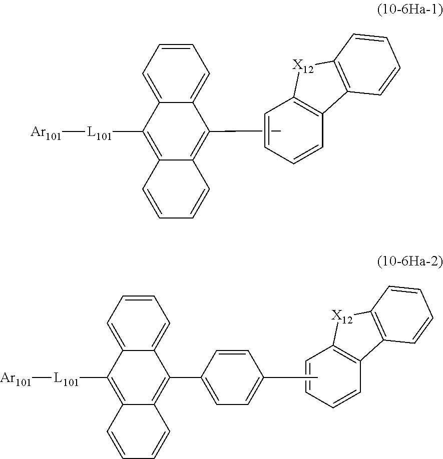

wherein in the formula (10-6Ha-1) and (10-6Ha-2),

L₁₀₁ and Ar₁₀₁ are as defined in the formula (10); and

X₁₂ is O or S.

In one embodiment, the compound (10) is a compound represented by the following formula (10-7):

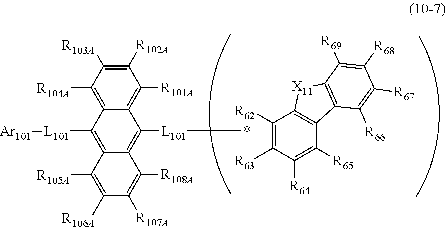

wherein in the formula (10-7),

L₁₀₁ and Ar₁₀₁ are as defined in the formula (10);

R_{101A} to R_{108A} are as defined in the formula (10-4);

X₁₁ is as defined in the formula (10-4); and

R₆₂ to R₆₉ are as defined in the formula (10-4), provided that any one pair of R₆₆ and R₆₇, R₆₇ and R₆₈, and R₆₈ and R₆₉ are bonded with each other to form a substituted or unsubstituted, saturated or unsaturated ring.

In one embodiment, the compound (10) is a compound represented by the following formula (10-7H):

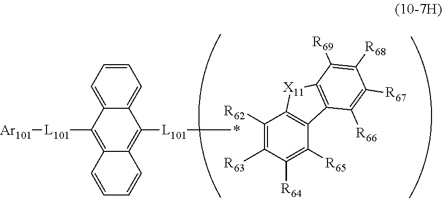

wherein in the formula (10-7H),

L₁₀₁ and Ar₁₀₁ are as defined in the formula (10);

X₁₁ is as defined in the formula (10-4); and

R₆₂ to R₆₉ are as defined in the formula (10-4), provided that any one pair of R₆₆ and R₆₇, R₆₇ and R₆₈, and R₆₈ and R₆₉ are bonded with each other to form a substituted or unsubstituted, saturated or unsaturated ring.

In one embodiment, the compound (10) is a compound represented by the following formula (10-8):

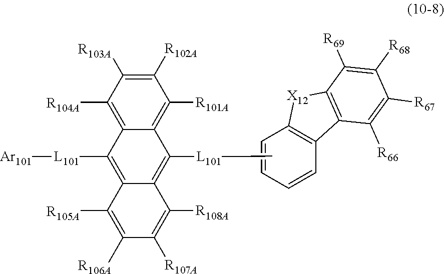

wherein in the formula (10-8),

L₁₀₁ and Ar₁₀₁ are as defined in the formula (10);

R_{101A} to R_{108A} are as defined in the formula (10-4);

X₁₂ is O or S; and

R₆₆ to R₆₉ are as defined in the formula (10-4), provided that any one pair of R₆₆ and R₆₇, R₆₇ and R₆₈, as well as R₆₈ and R₆₉ are bonded with each other to form a substituted or unsubstituted, saturated or unsaturated ring.

In one embodiment, the compound represented by the formula (10-8) is a compound represented by the following formula (10-8H):

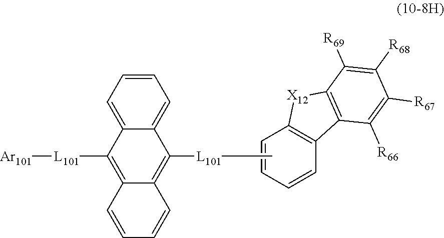

In the formula (10-8H), L₁₀₁ and Ar₁₀₁ are as defined in the formula (10).

R₆₆ to R₆₉ are as defined in the formula (10-4), provided that any one pair of R₆₆ and R₆₇, R₆₇ and R₆₈, as well as R₆₈ and R₆₉ are bonded with each other to form a substituted or unsubstituted, saturated or unsaturated ring. Any one pair of R₆₆ and R₆₇, R₆₇ and R₆₈, as well as R₆₈ and R₆₉ may preferably be bonded with each other to form an unsubstituted benzene ring; and

X₁₂ is O or S.

In one embodiment, as for the compound represented by the formula (10-7), (10-8) or (10-8H), any one pair of R₆₆ and R₆₇, R₆₇ and R₆₈, as well as R₆₈ and R₆₉ are bonded with each other to form a ring represented by the following formula (10-8-1) or (10-8-2), and R₆₆ to R₆₉ that do not form the ring represented by the formula (10-8-1) or (10-8-2) do not form a substituted or unsubstituted, saturated or unsaturated ring.

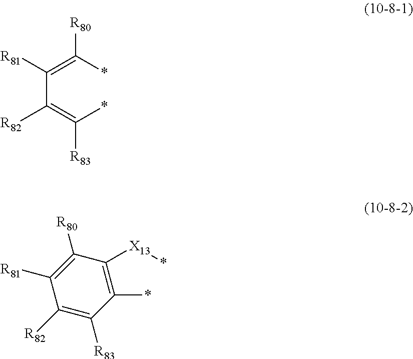

wherein in the formulas (10-8-1) and (10-8-2),

the two atomic bondings * are independently bonded with one pair of R₆₆ and R₆₇, R₆₇ and R₆₈, or R₆₈ and R₆₉;

R₈₀ to R₈₃ are independently a hydrogen atom, a substituted or unsubstituted alkyl group including 1 to 50 carbon atoms or a substituted or unsubstituted aryl group including 6 to 50 ring carbon atoms; and

X₁₃ is O or S.

In one embodiment, the compound (10) is a compound represented by the following formula (10-9):

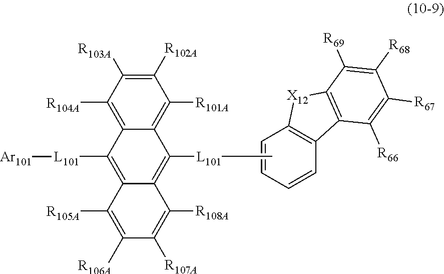

wherein in the formula (10-9),

L₁₀₁ and Ar₁₀₁ are as defined in the formula (10);

R_{101A} to R_{108A} are as defined in the formula (10-4);

R₆₆ to R₆₉ are as defined in the formula (10-4), provided that R₆₆ and R₆₇, R₆₇ and R₆₈, as well as R₆₈ and R₆₉ are not bonded with each other and do not form a substituted or unsubstituted, saturated or unsaturated ring; and

X₁₂ is O or S.

In one embodiment, the compound (10) is selected from the group consisting of compounds represented by the following formulas (10-10-1) to (10-10-4).

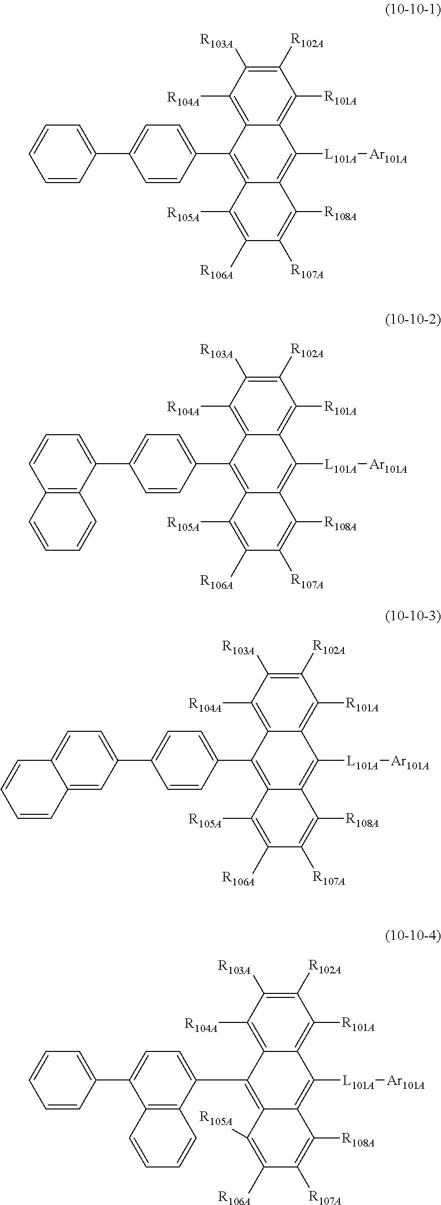

In the formulas (10-10-1H) to (10-10-4H), L_{101A} and Ar_{101A} are as defined in the formula (10-3).

As for the compound represented by the formula (10), the following compounds can be given as specific examples.

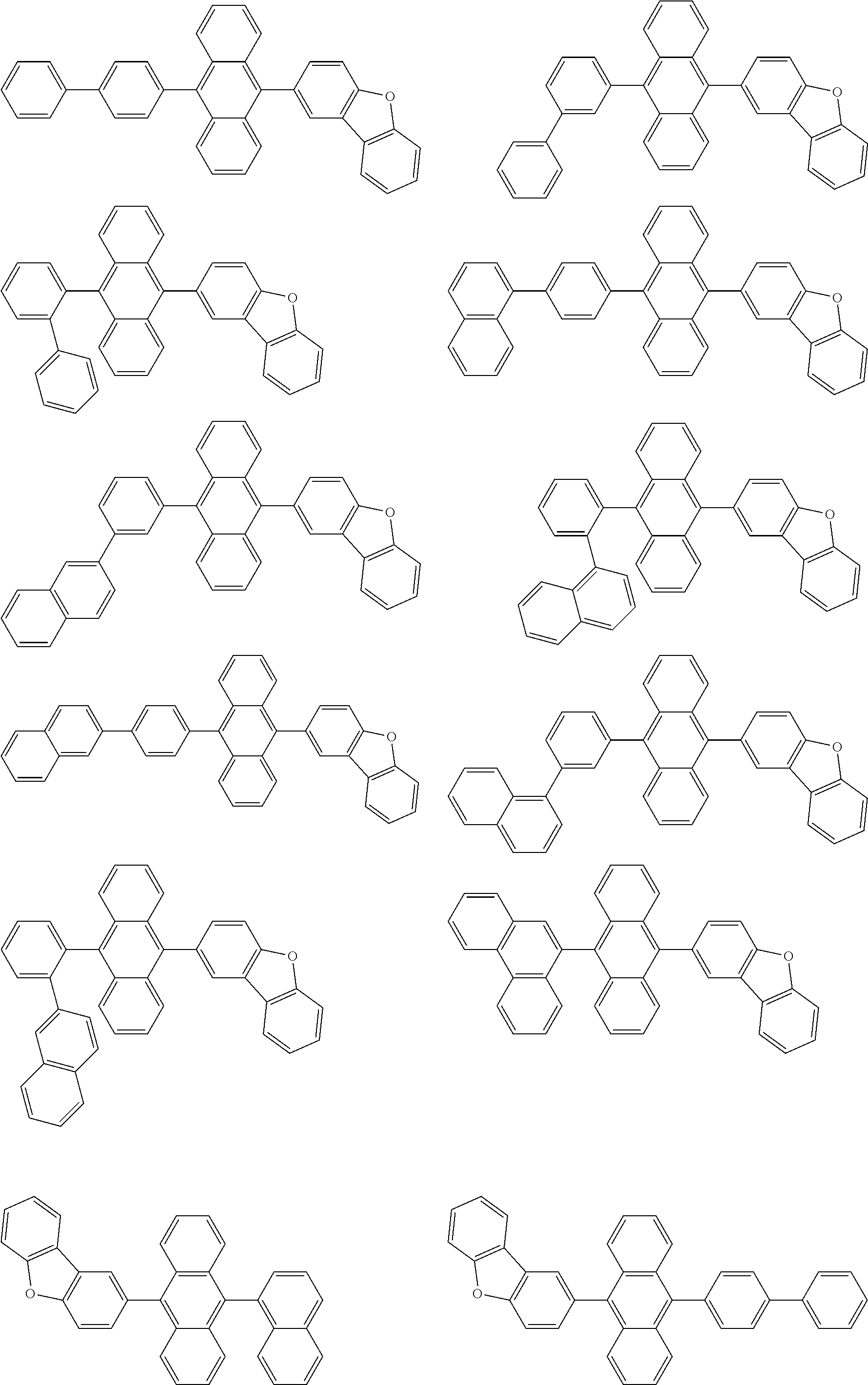

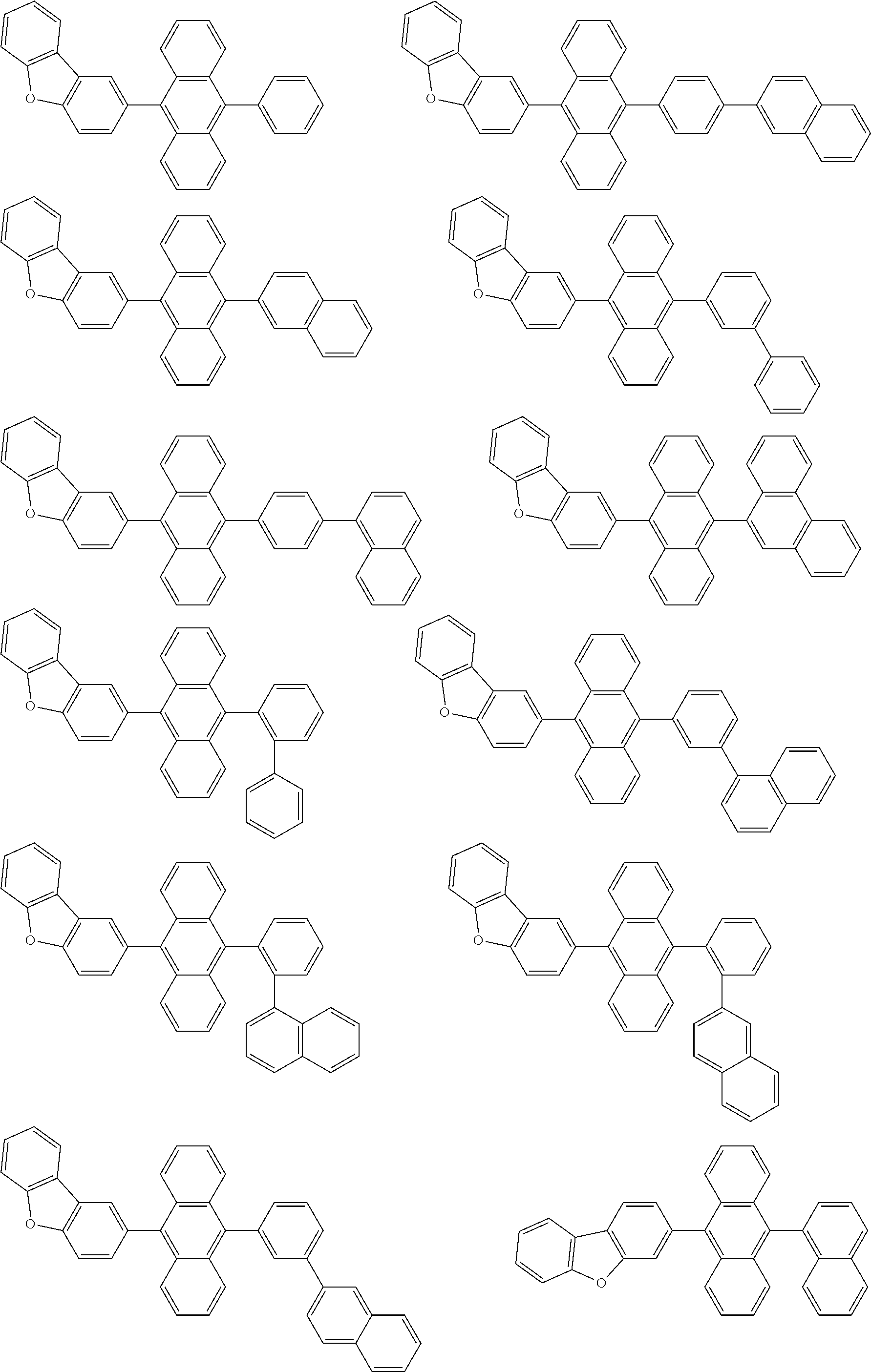

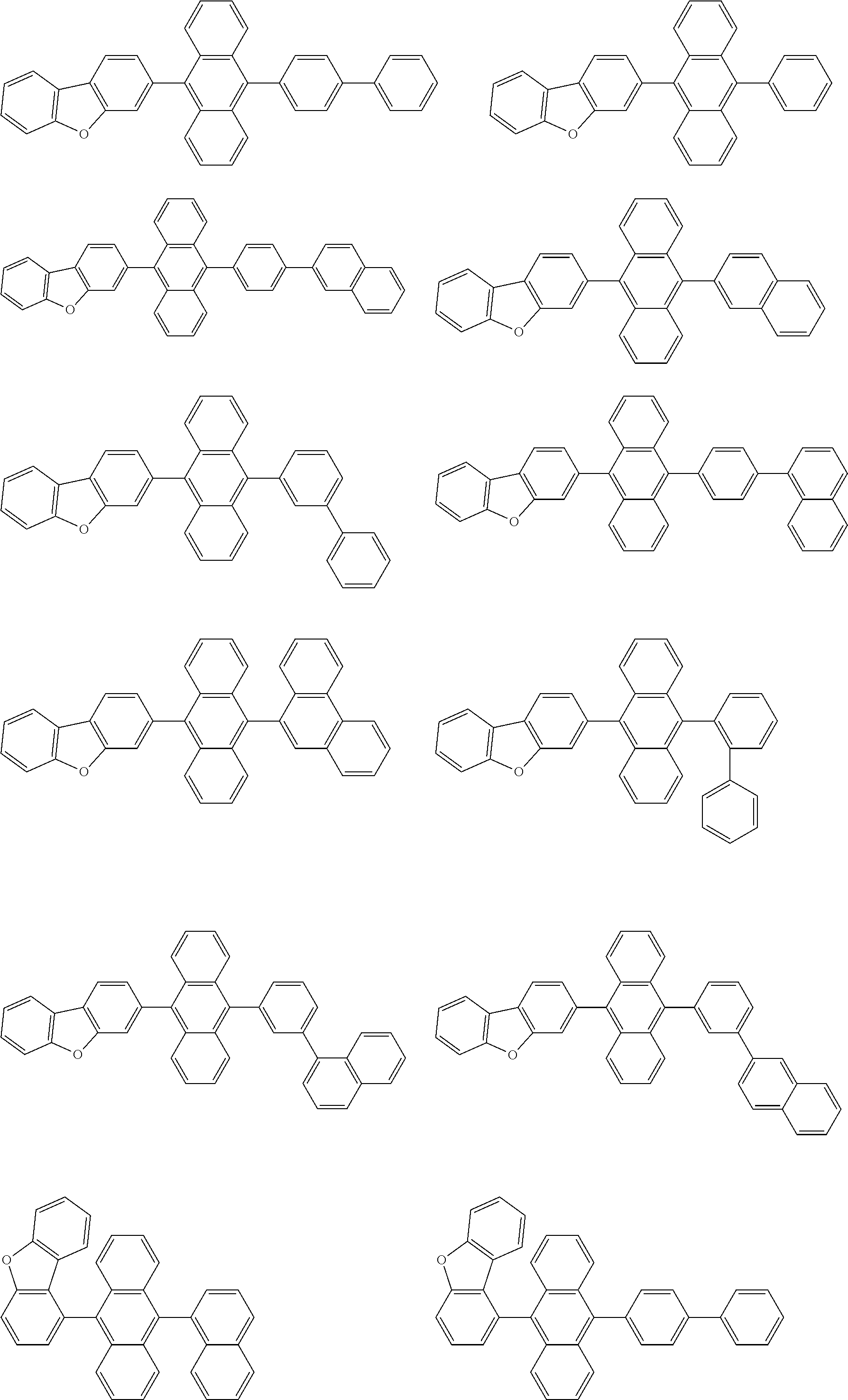

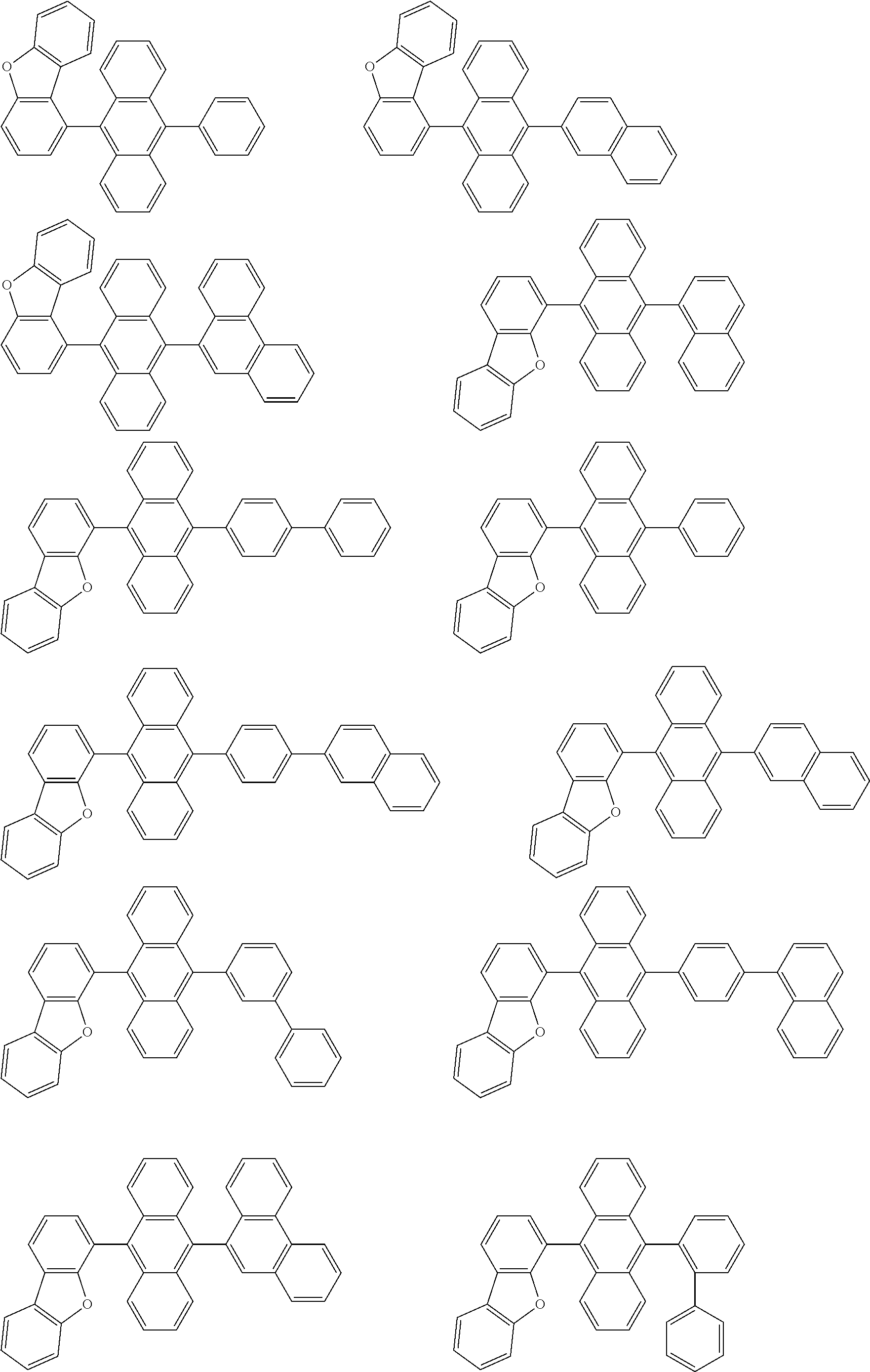

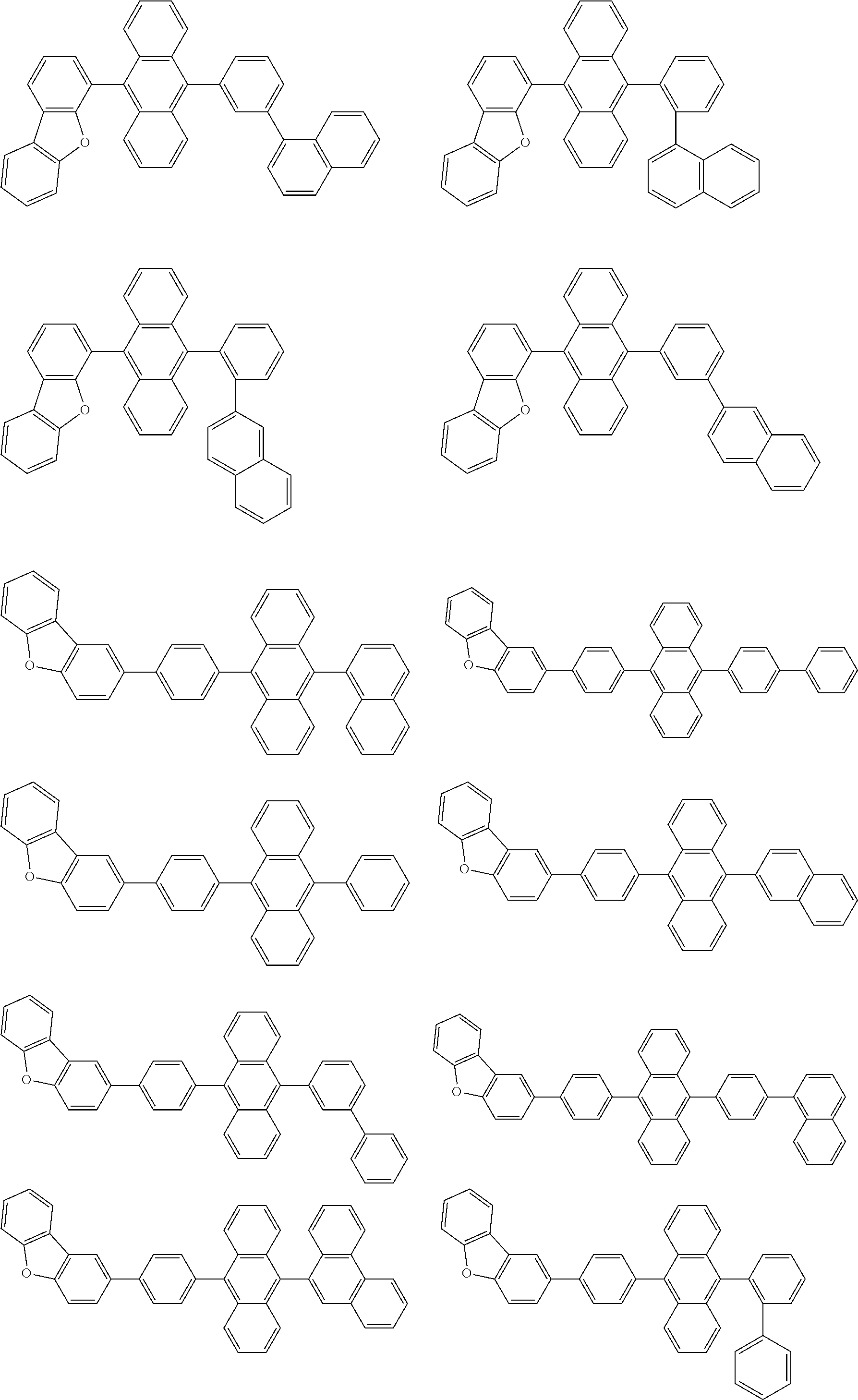

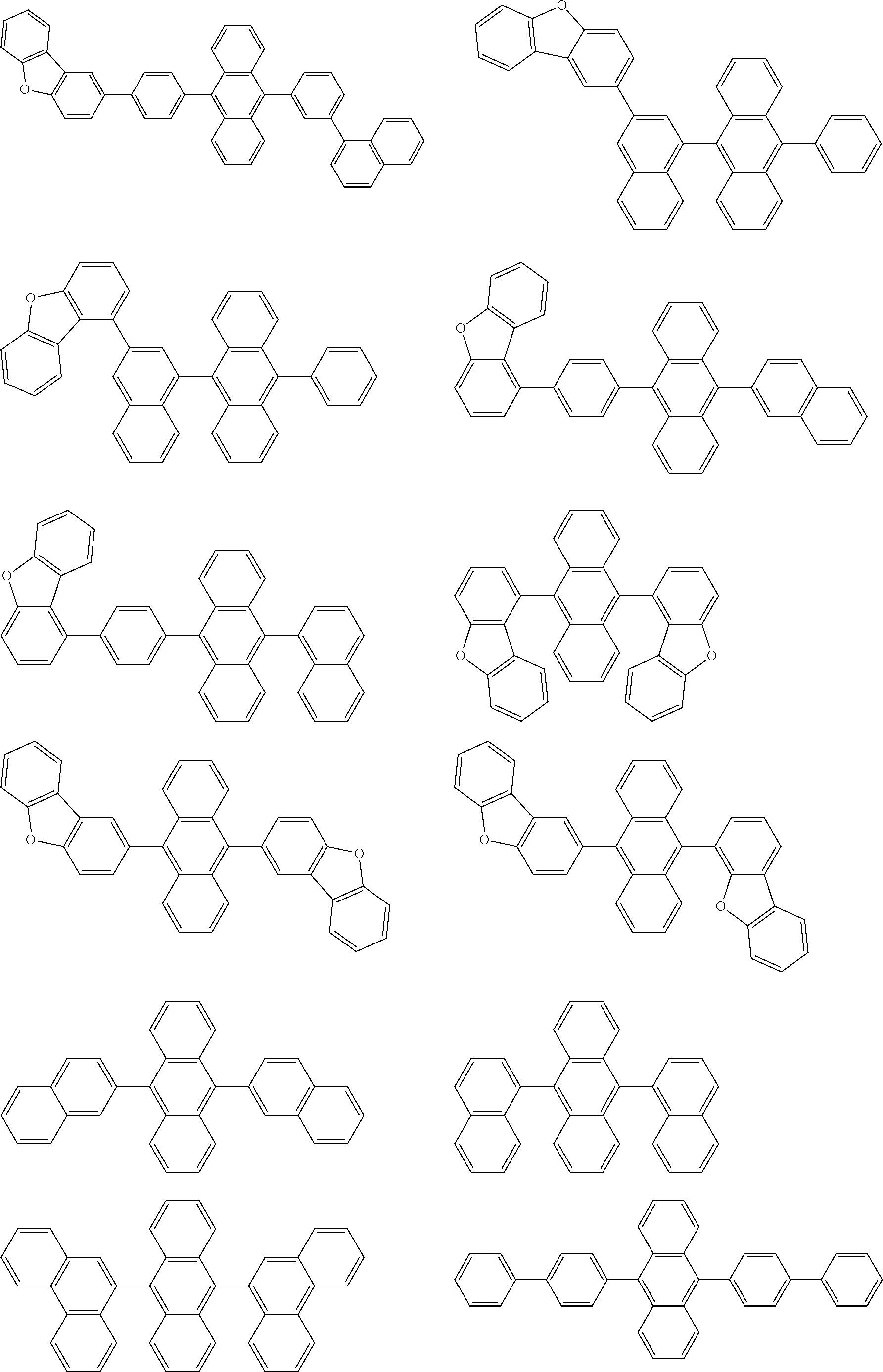

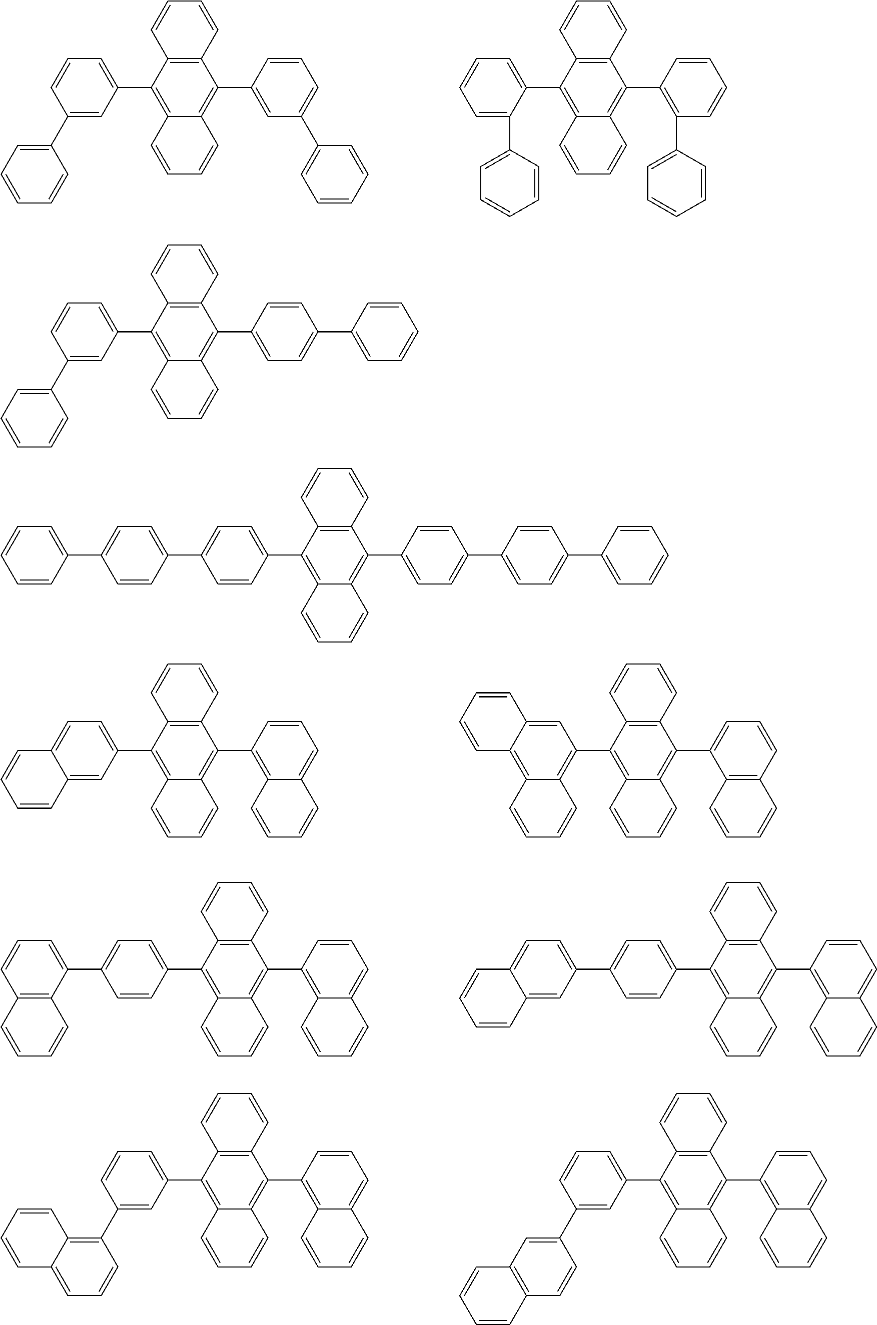

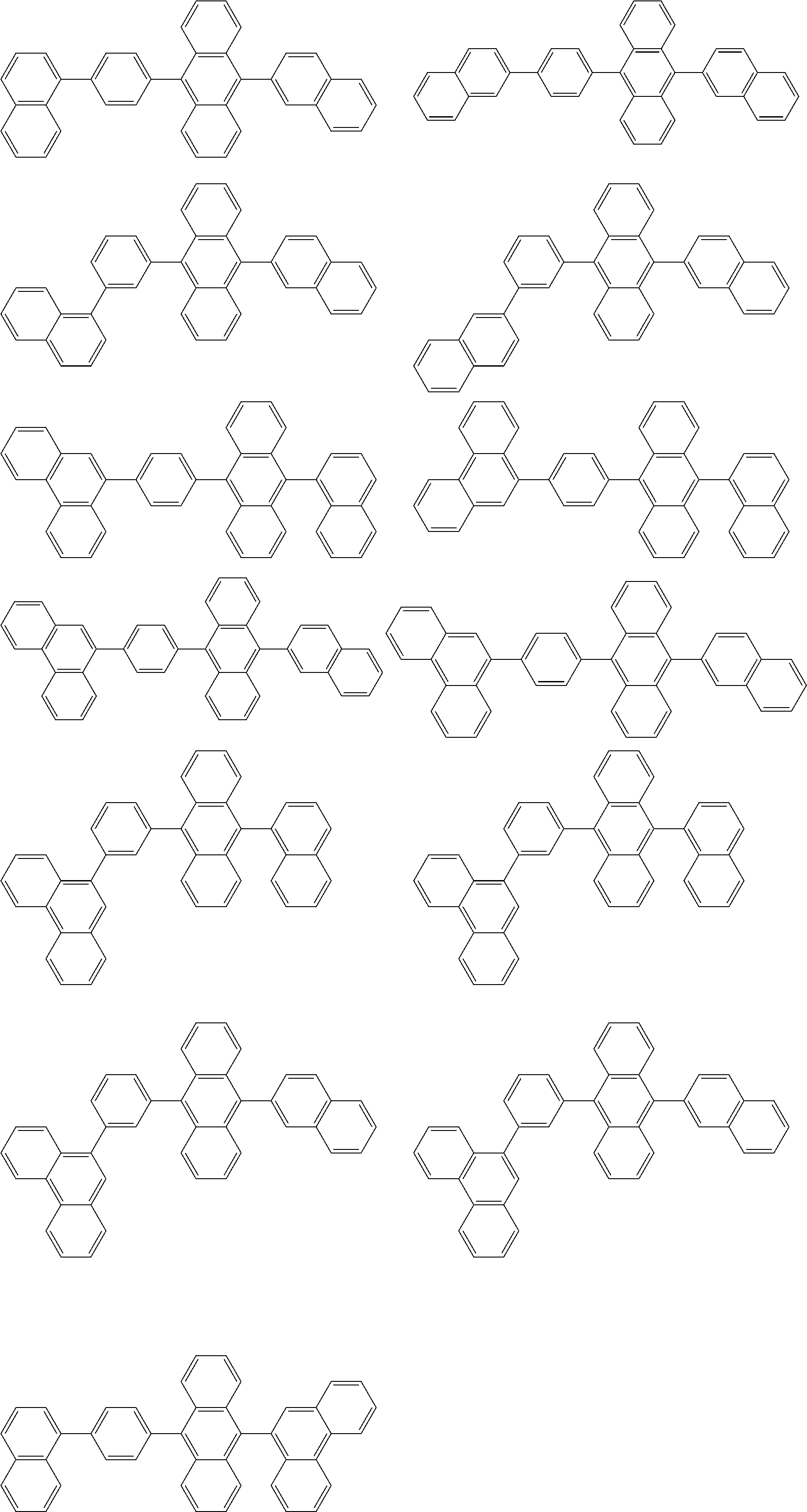

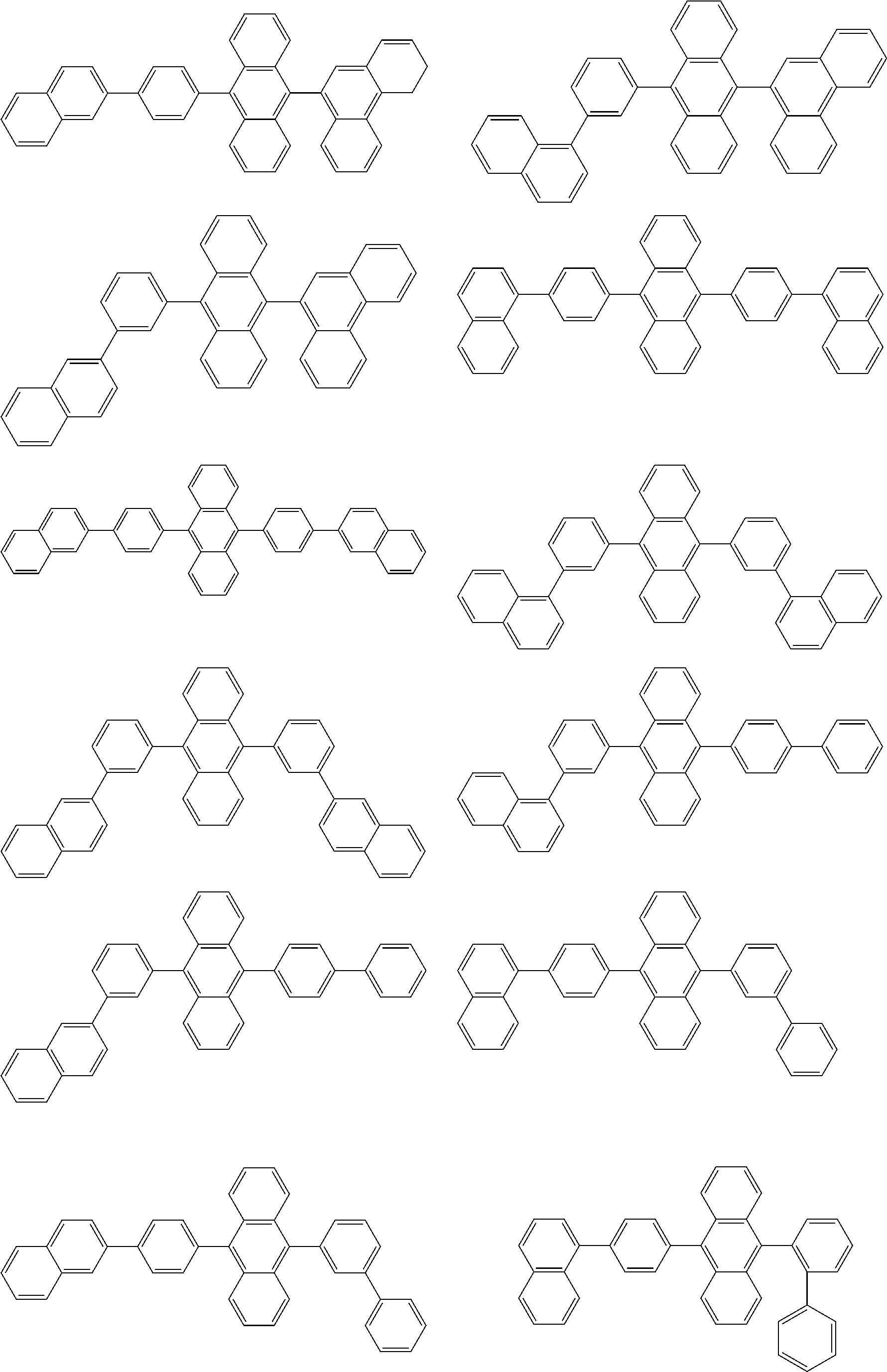

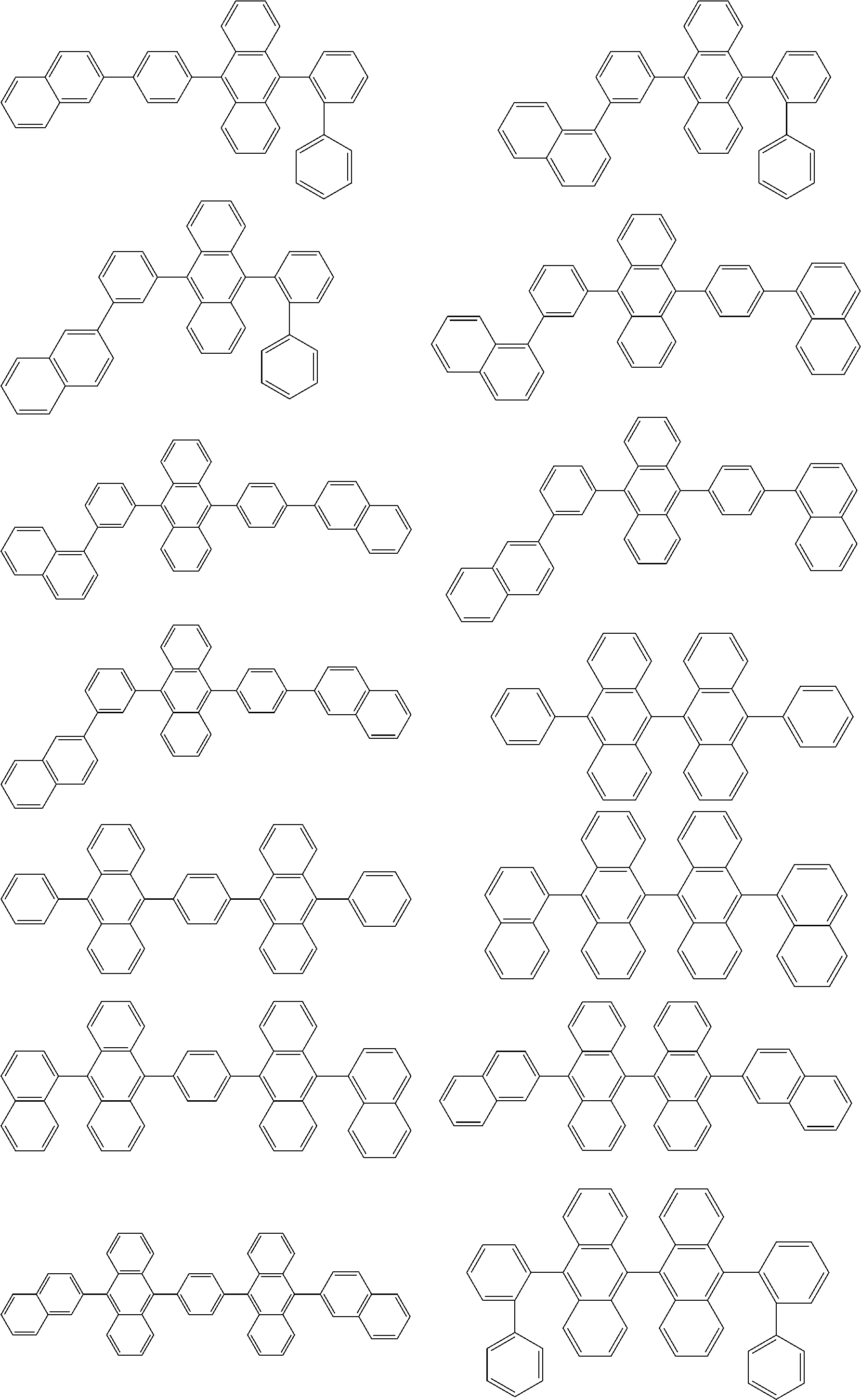

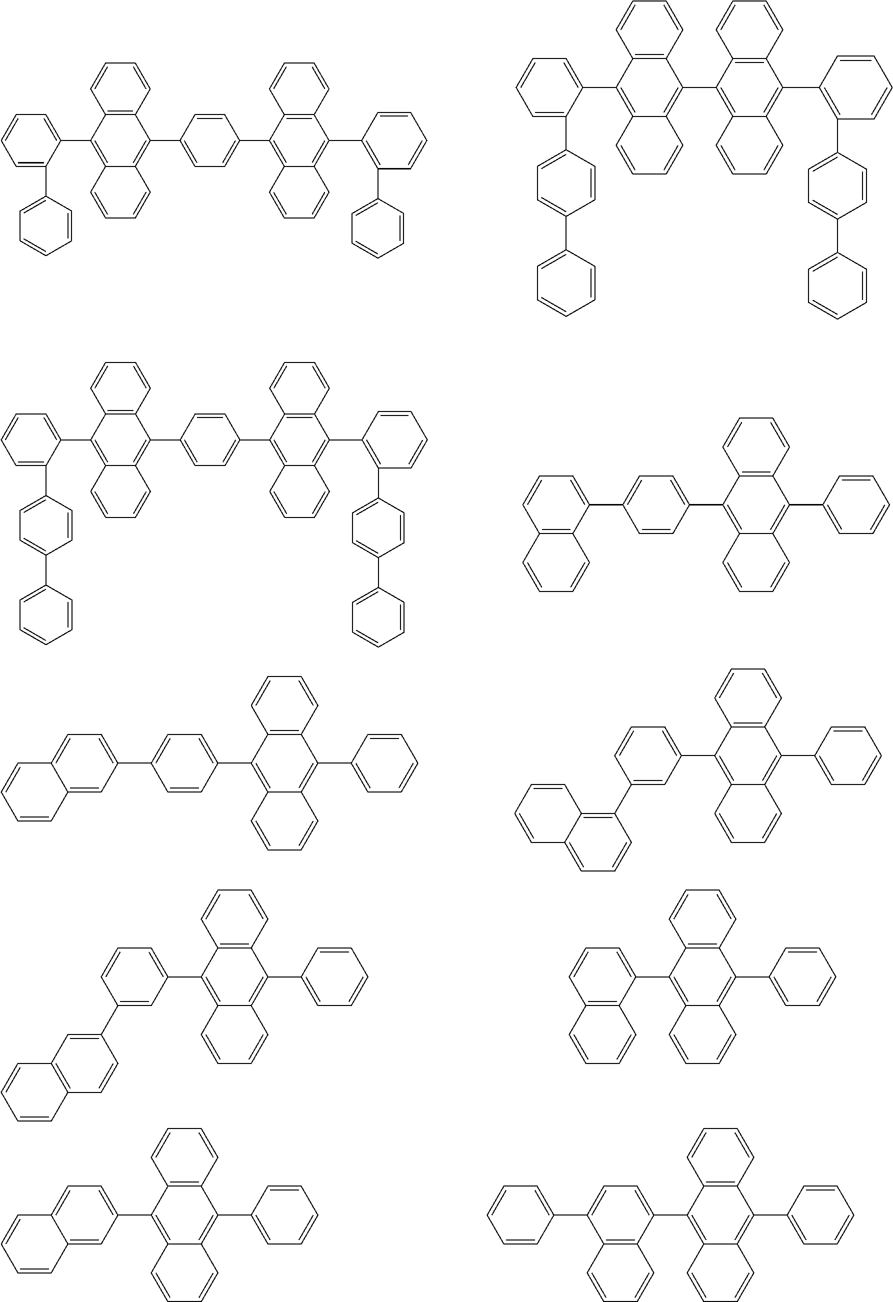

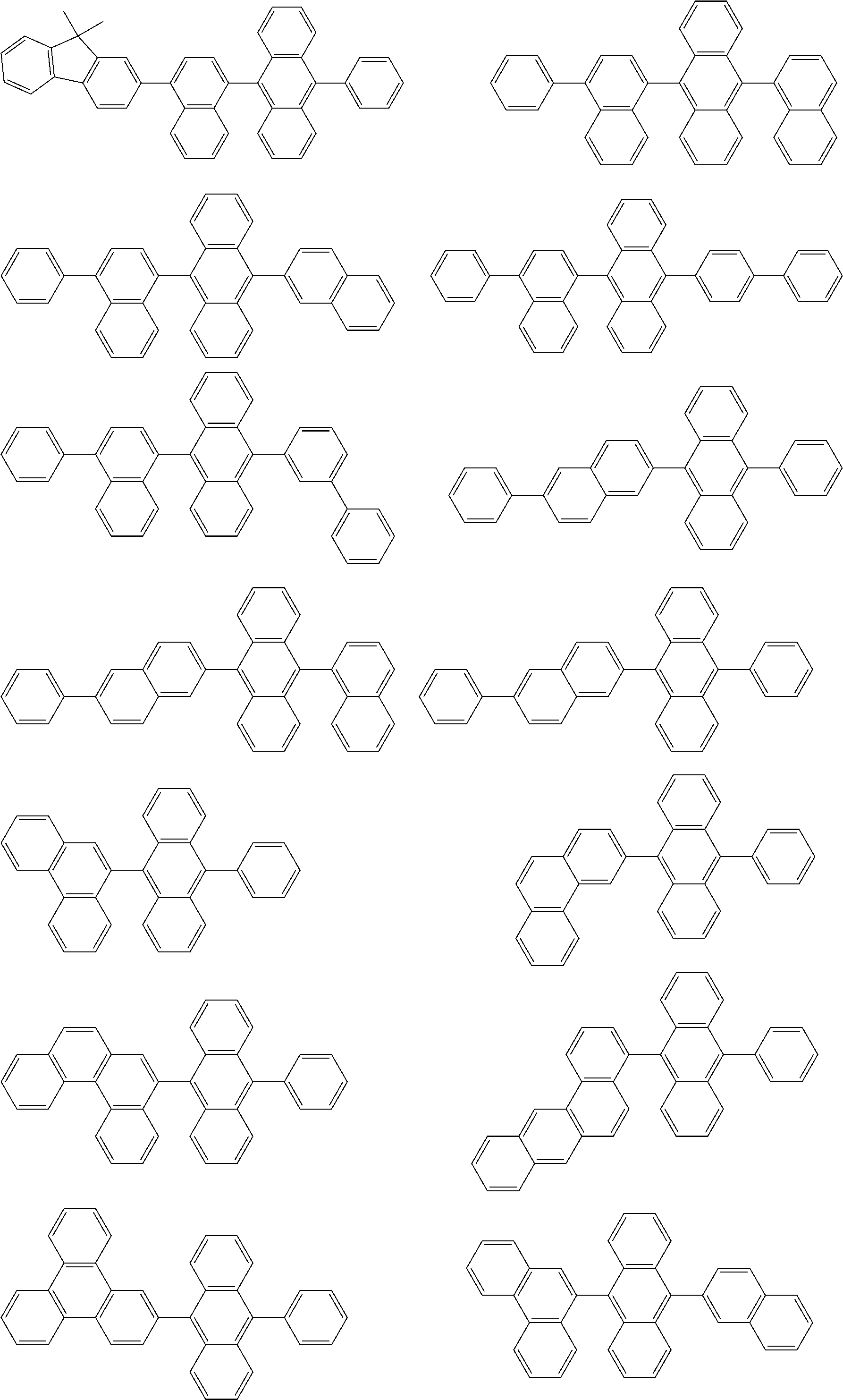

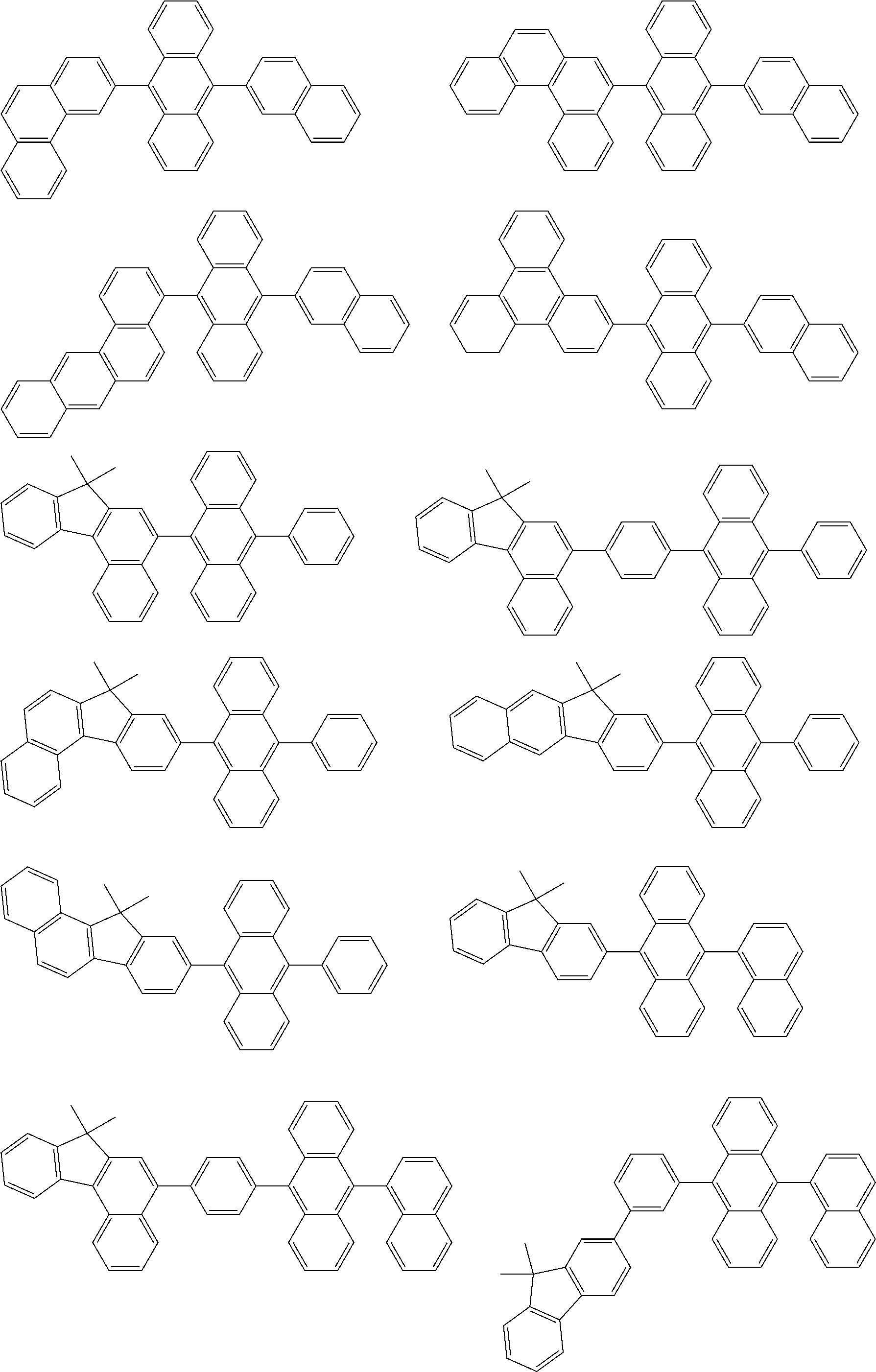

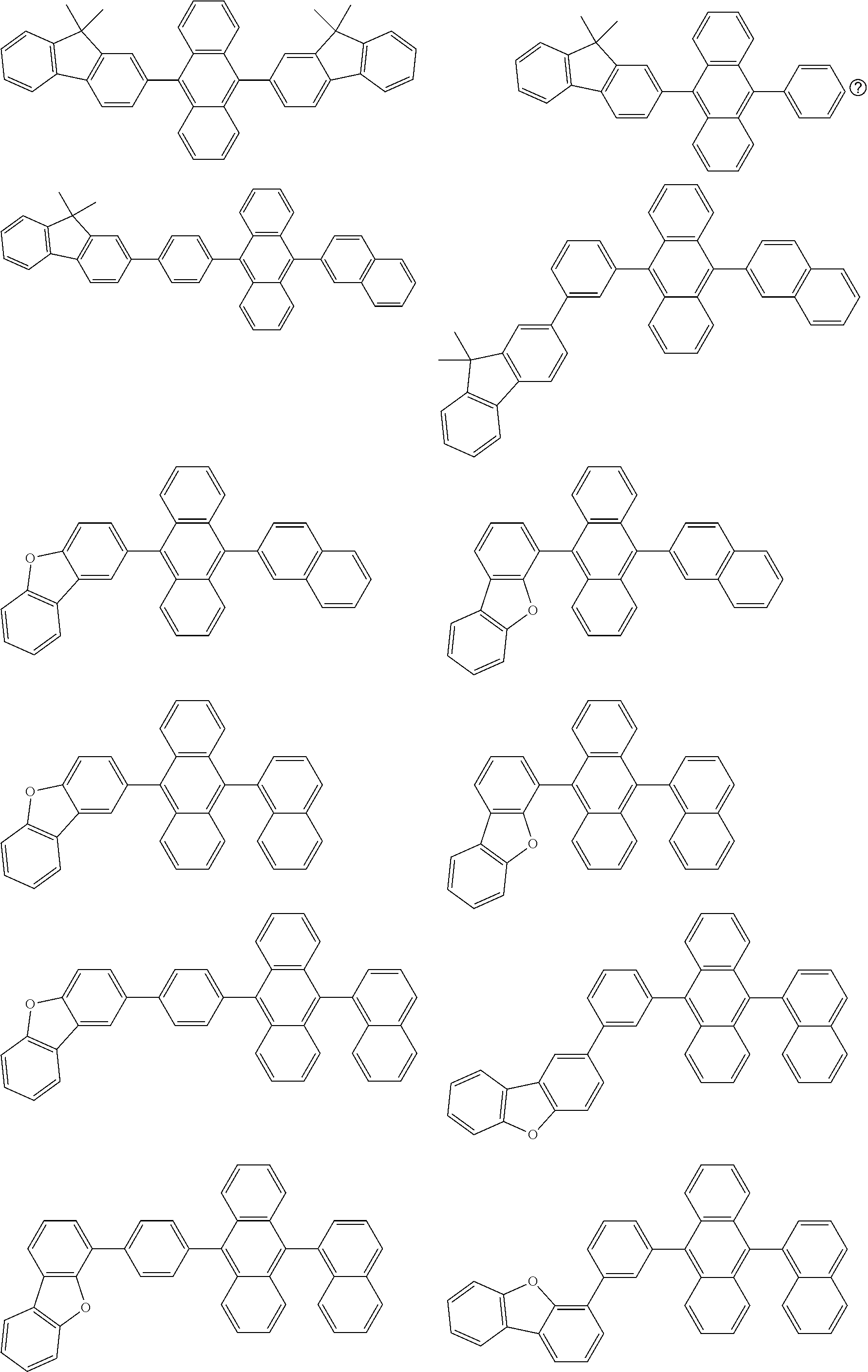

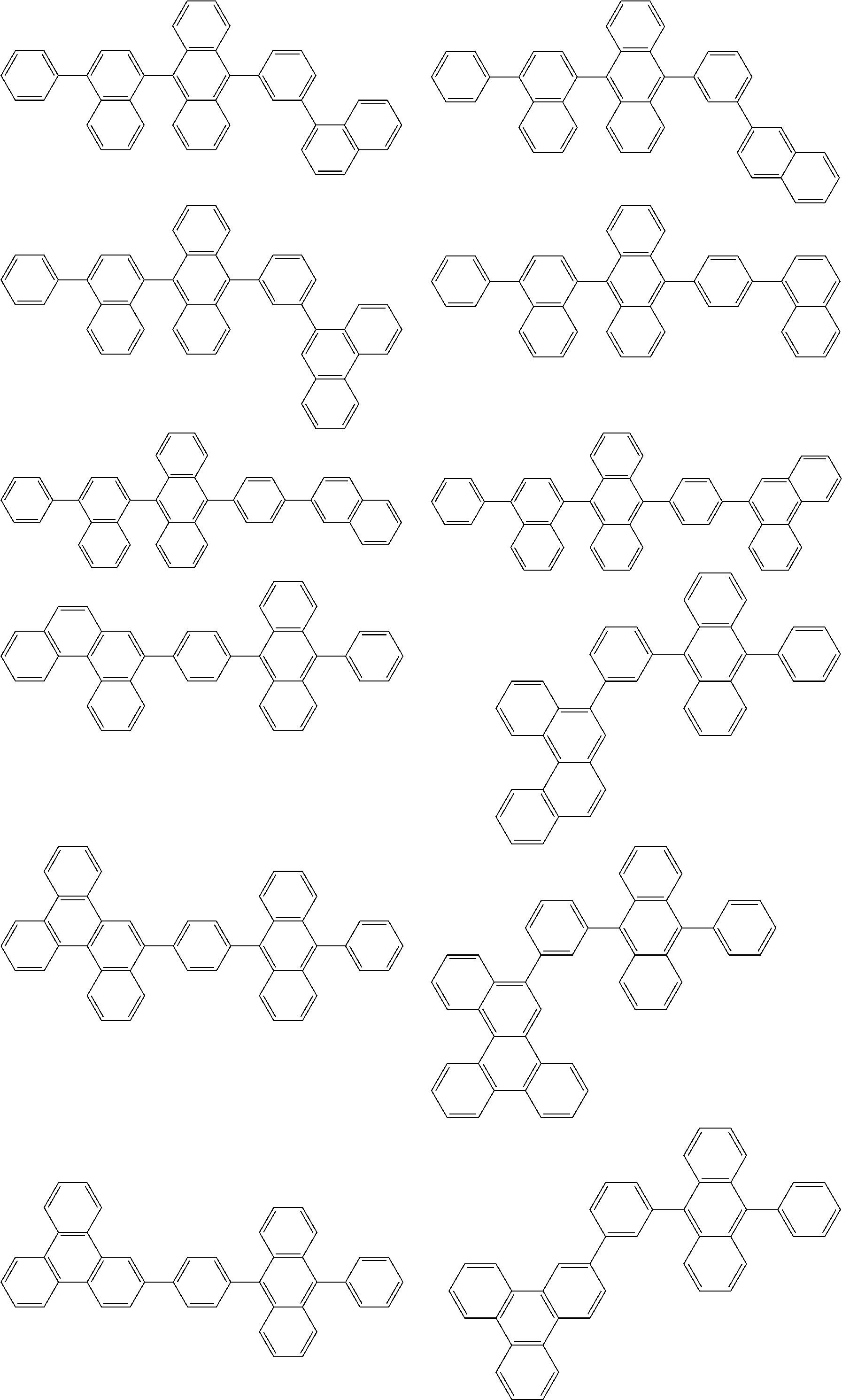

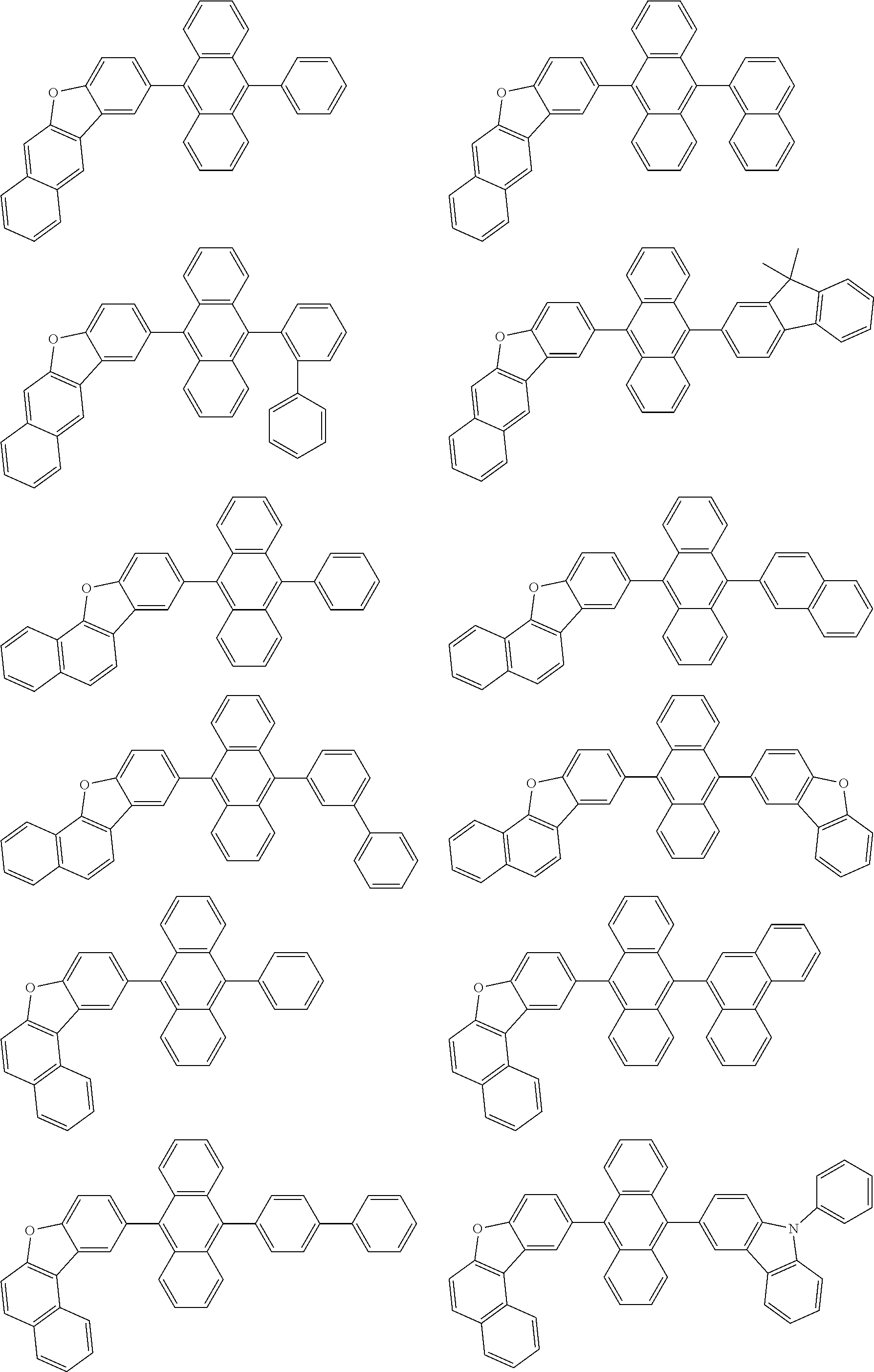

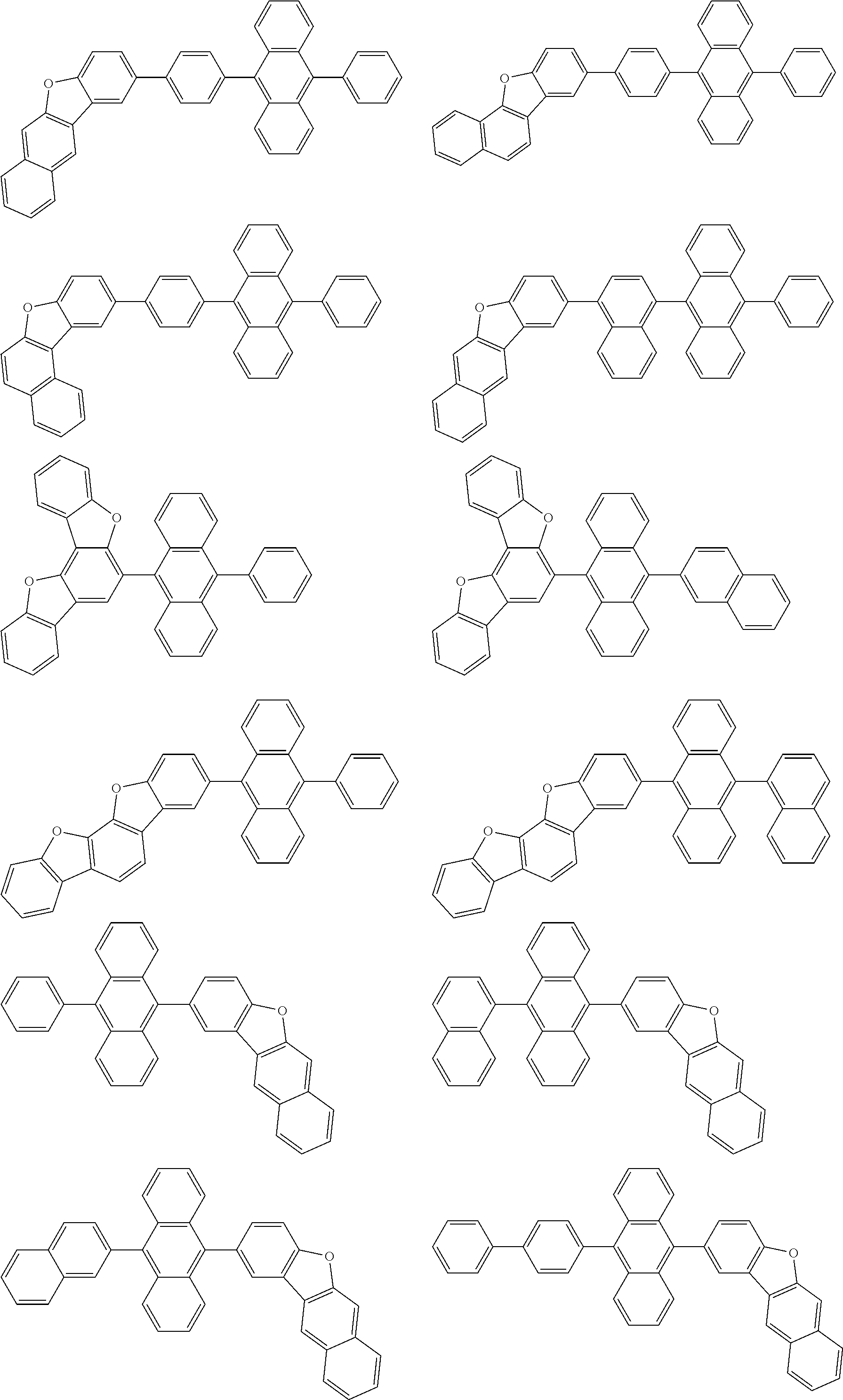

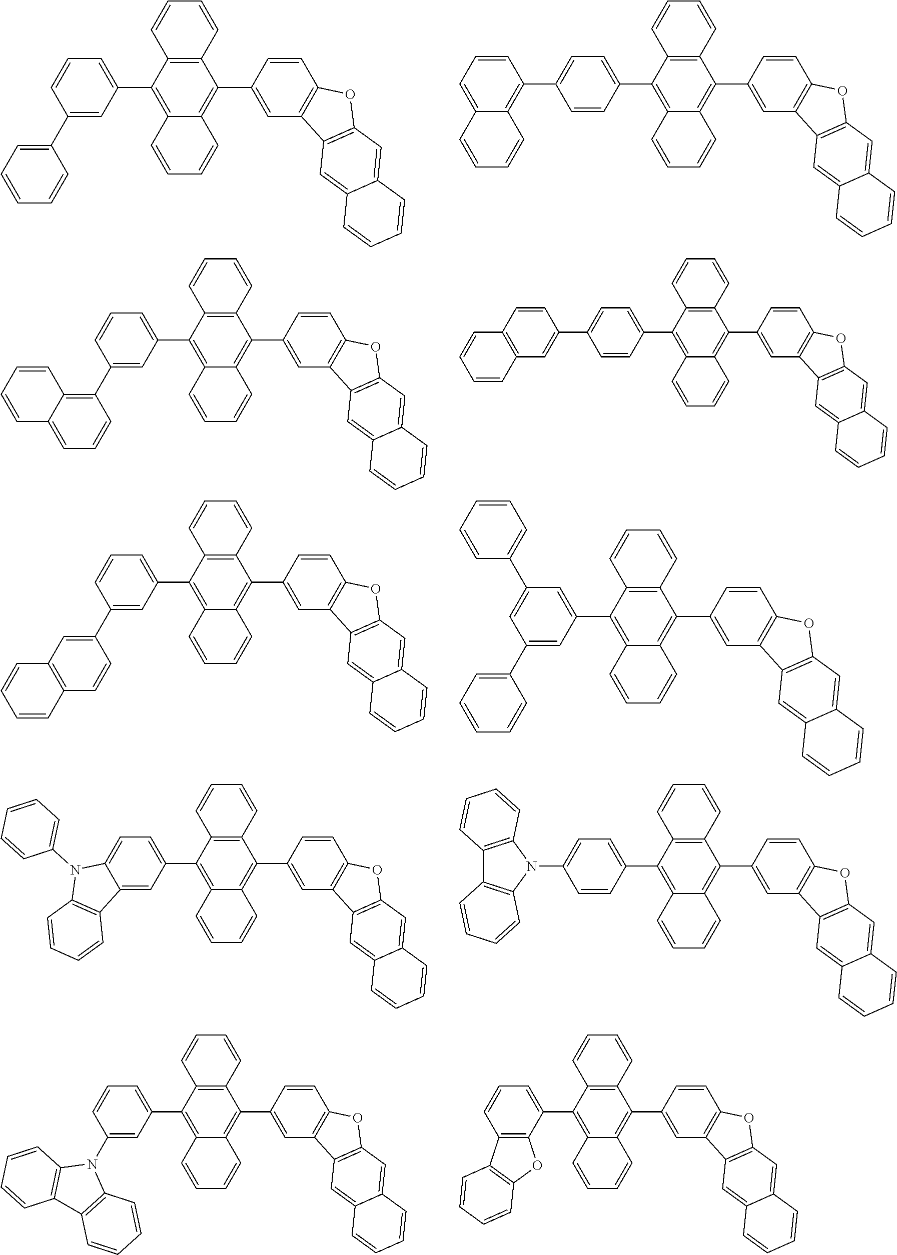

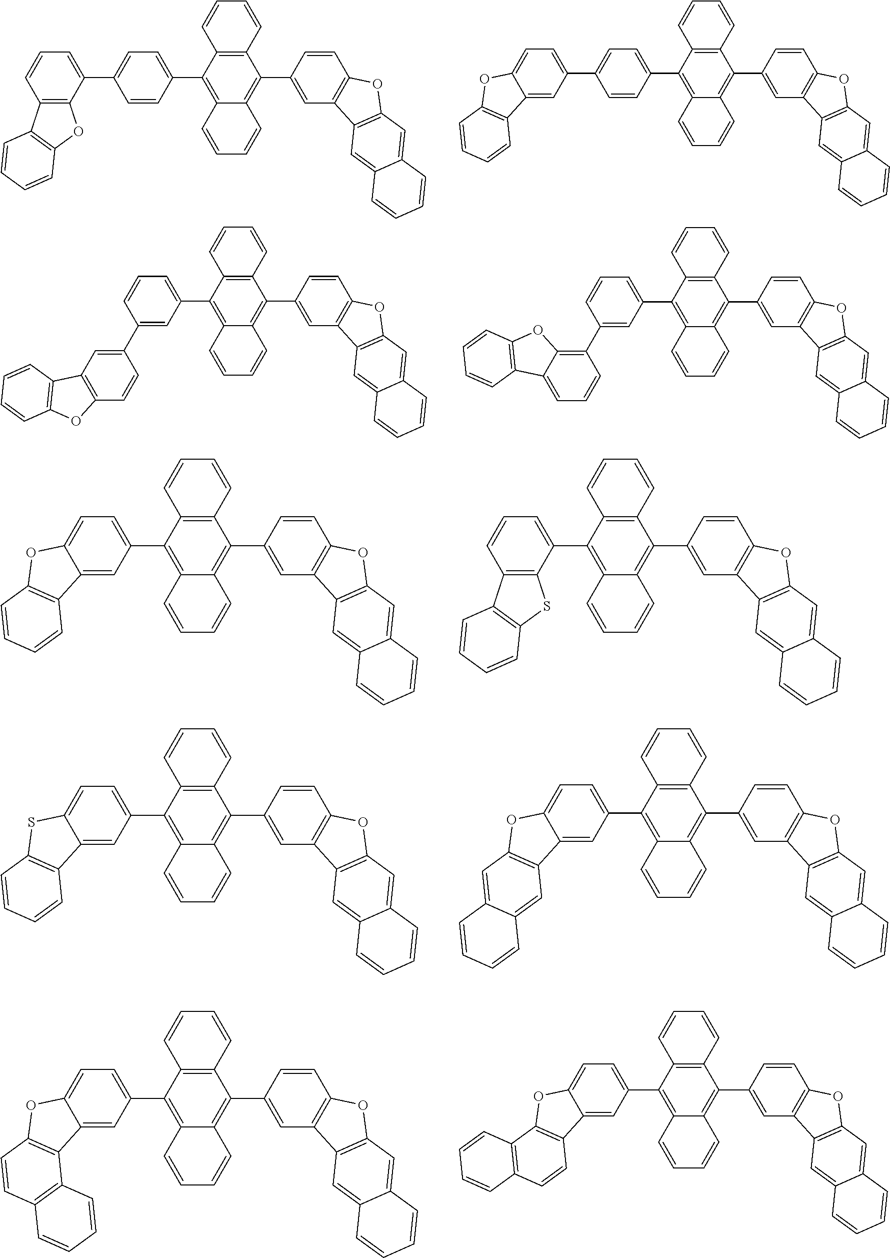

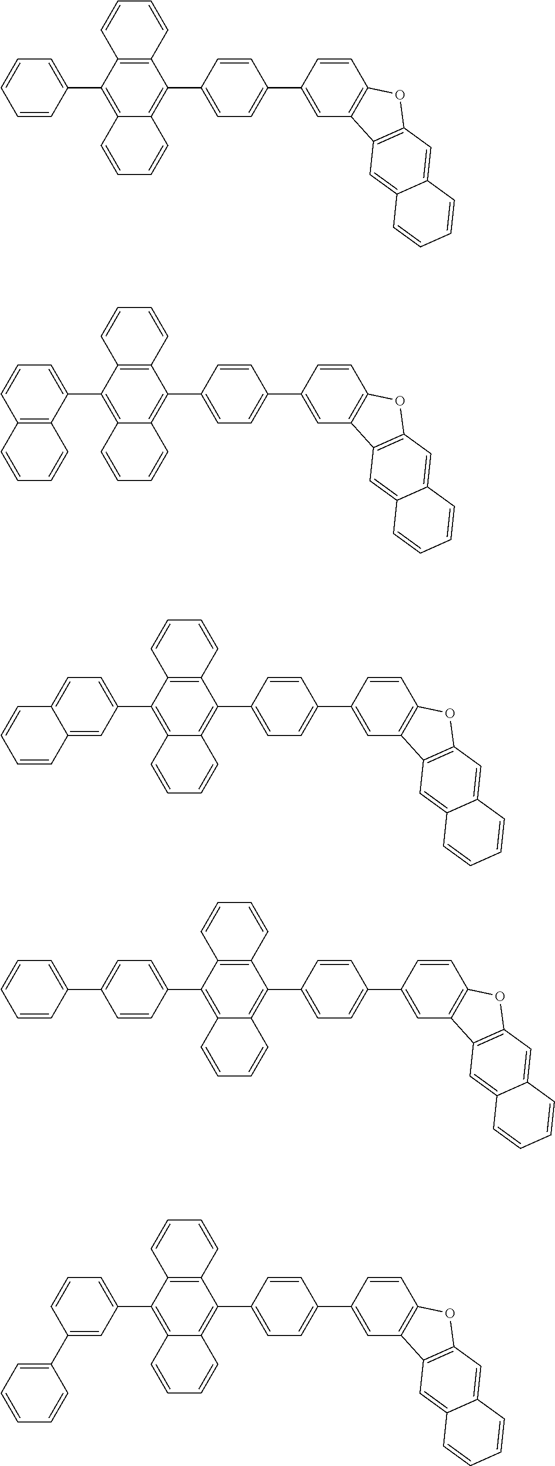 [SMILES: c1ccc(-c2c3ccccc3c(-c3ccc(-c4ccc5oc6cc7ccccc7cc6c5c4)cc3)c3ccccc23)cc1 ; c1ccc2cc3c(cc2c1)oc1ccc(-c2ccc(-c4c5ccccc5c(-c5cccc6ccccc56)c5ccccc45)cc2)cc13 ; c1ccc2cc(-c3c4ccccc4c(-c4ccc(-c5ccc6oc7cc8ccccc8cc7c6c5)cc4)c4ccccc34)ccc2c1 ; c1ccc(-c2ccc(-c3c4ccccc4c(-c4ccc(-c5ccc6oc7cc8ccccc8cc7c6c5)cc4)c4ccccc34)cc2)cc1 ; c1ccc(-c2cccc(-c3c4ccccc4c(-c4ccc(-c5ccc6oc7cc8ccccc8cc7c6c5)cc4)c4ccccc34)c2)cc1]

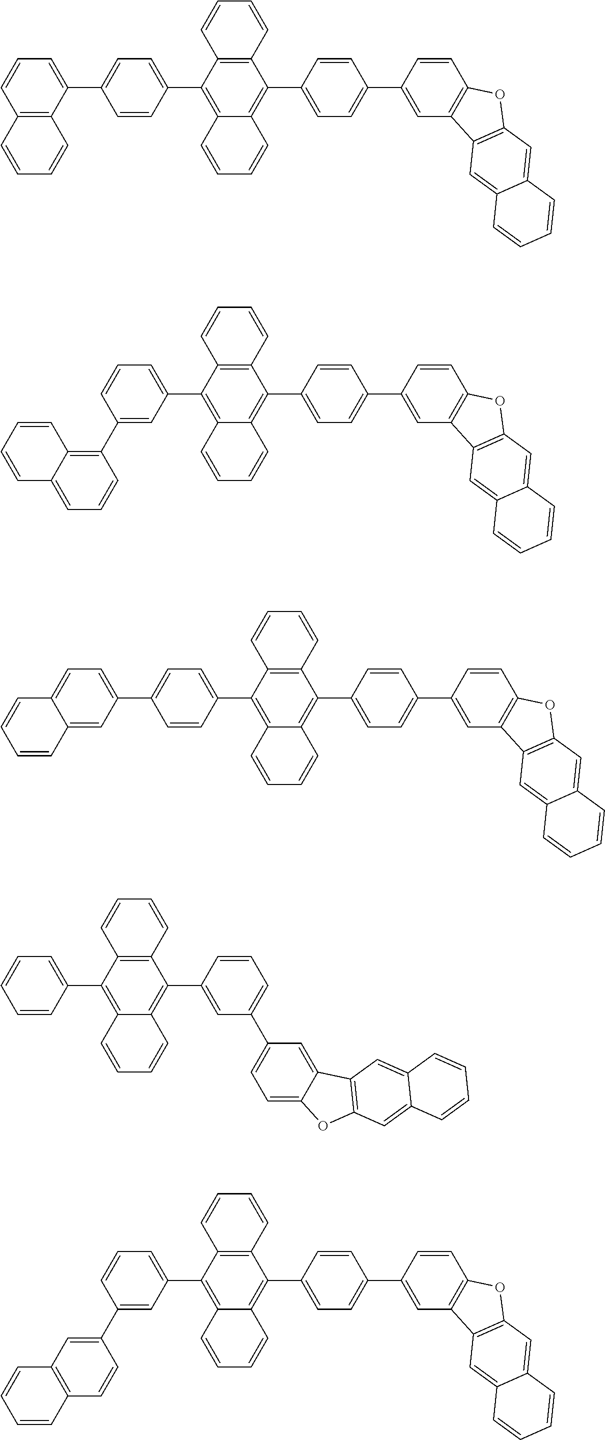 [SMILES: c1ccc2cc3c(cc2c1)oc1ccc(-c2ccc(-c4c5ccccc5c(-c5ccc(-c6cccc7ccccc67)cc5)c5ccccc45)cc2)cc13 ; c1cc(-c2cccc3ccccc23)cc(-c2c3ccccc3c(-c3ccc(-c4ccc5oc6cc7ccccc7cc6c5c4)cc3)c3ccccc23)c1 ; c1ccc2cc(-c3ccc(-c4c5ccccc5c(-c5ccc(-c6ccc7oc8cc9ccccc9cc8c7c6)cc5)c5ccccc45)cc3)ccc2c1 ; c1ccc(-c2c3ccccc3c(-c3cccc(-c4ccc5oc6cc7ccccc7cc6c5c4)c3)c3ccccc23)cc1 ; c1cc(-c2ccc3ccccc3c2)cc(-c2c3ccccc3c(-c3ccc(-c4ccc5oc6cc7ccccc7cc6c5c4)cc3)c3ccccc23)c1]

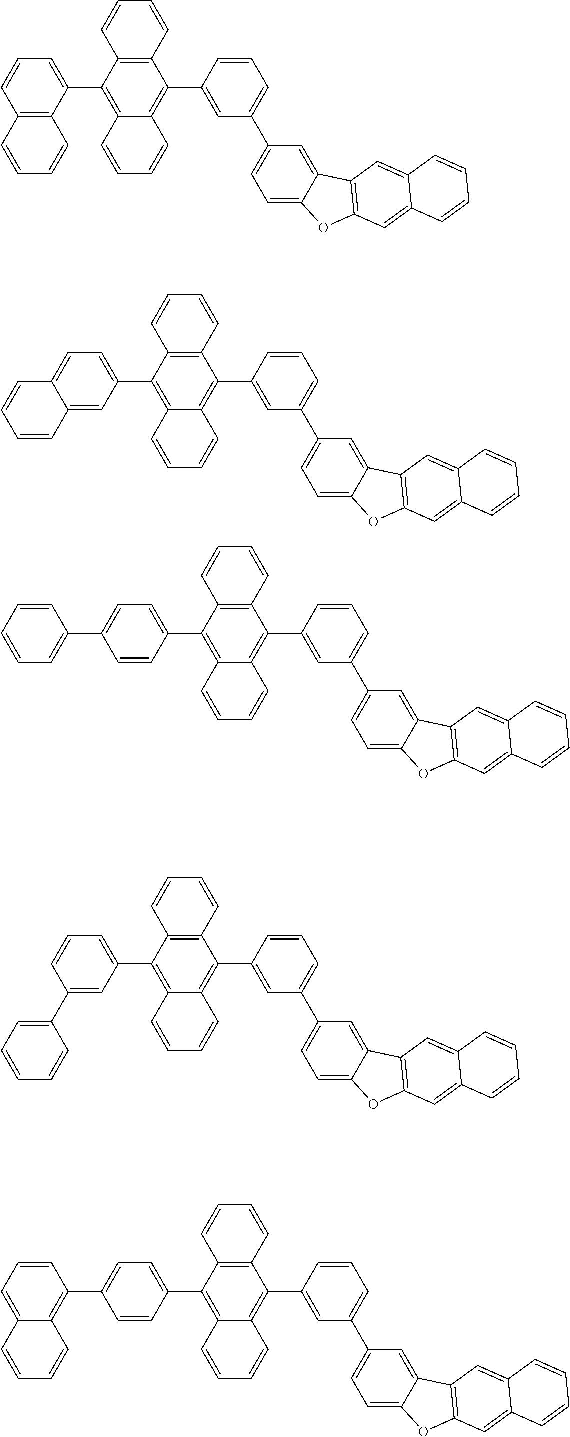 [SMILES: c1cc(-c2ccc3oc4cc5ccccc5cc4c3c2)cc(-c2c3ccccc3c(-c3cccc4ccccc34)c3ccccc23)c1 ; c1cc(-c2ccc3oc4cc5ccccc5cc4c3c2)cc(-c2c3ccccc3c(-c3ccc4ccccc4c3)c3ccccc23)c1 ; c1ccc(-c2ccc(-c3c4ccccc4c(-c4cccc(-c5ccc6oc7cc8ccccc8cc7c6c5)c4)c4ccccc34)cc2)cc1 ; c1ccc(-c2cccc(-c3c4ccccc4c(-c4cccc(-c5ccc6oc7cc8ccccc8cc7c6c5)c4)c4ccccc34)c2)cc1 ; c1cc(-c2ccc3oc4cc5ccccc5cc4c3c2)cc(-c2c3ccccc3c(-c3ccc(-c4cccc5ccccc45)cc3)c3ccccc23)c1]

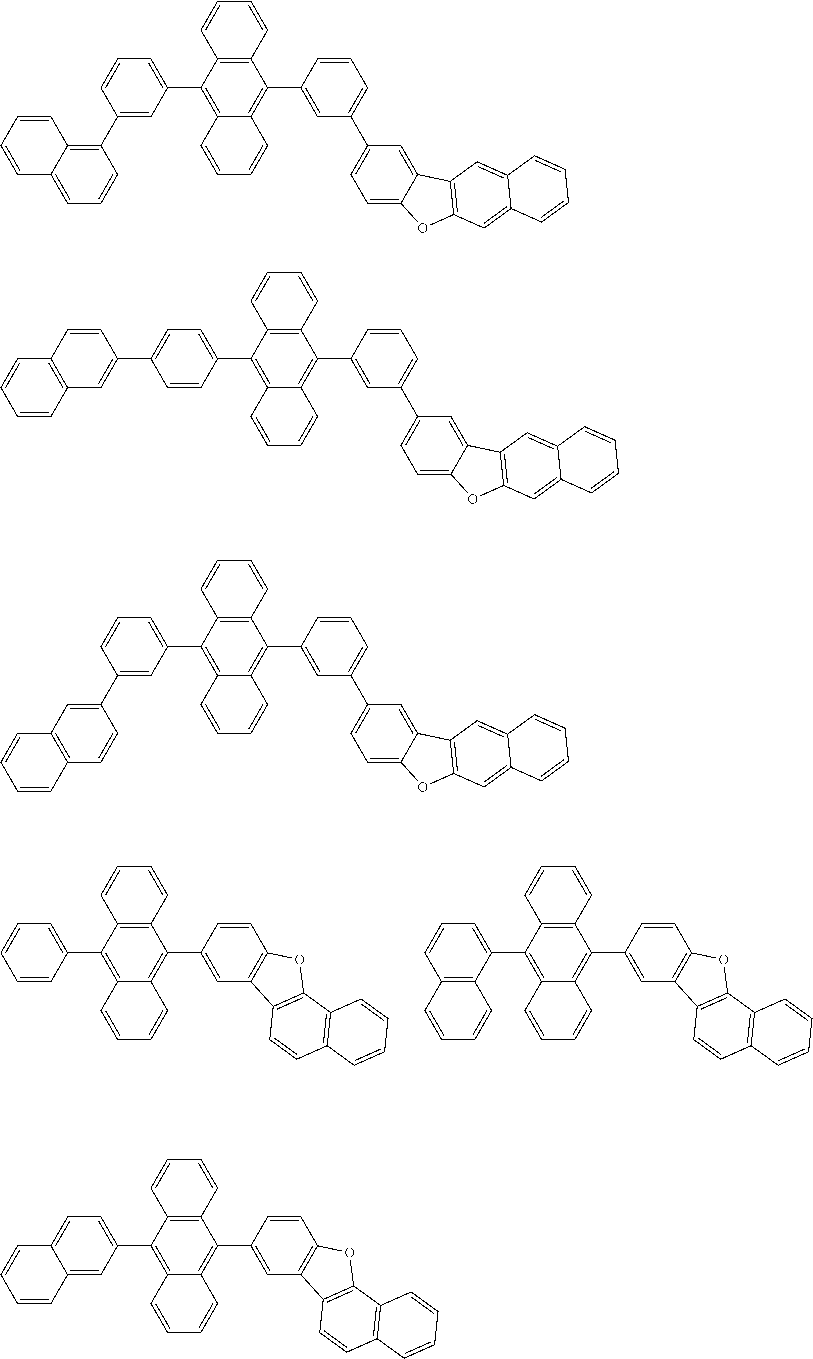

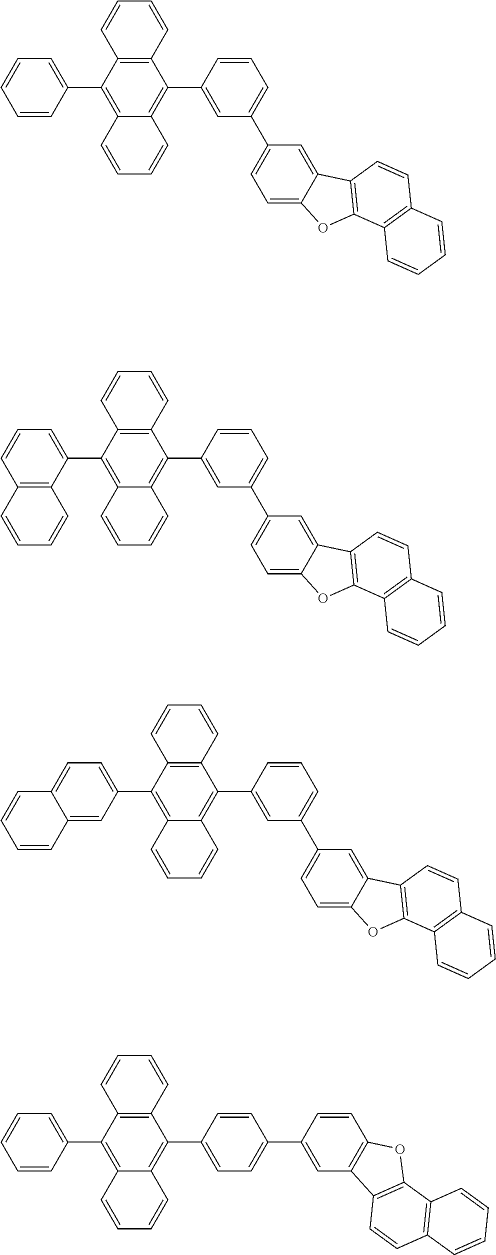 [SMILES: c1ccc(-c2c3ccccc3c(-c3cccc(-c4ccc5oc6c7ccccc7ccc6c5c4)c3)c3ccccc23)cc1 ; c1cc(-c2ccc3oc4c5ccccc5ccc4c3c2)cc(-c2c3ccccc3c(-c3cccc4ccccc34)c3ccccc23)c1 ; c1cc(-c2ccc3oc4c5ccccc5ccc4c3c2)cc(-c2c3ccccc3c(-c3ccc4ccccc4c3)c3ccccc23)c1 ; c1ccc(-c2c3ccccc3c(-c3ccc(-c4ccc5oc6c7ccccc7ccc6c5c4)cc3)c3ccccc23)cc1]

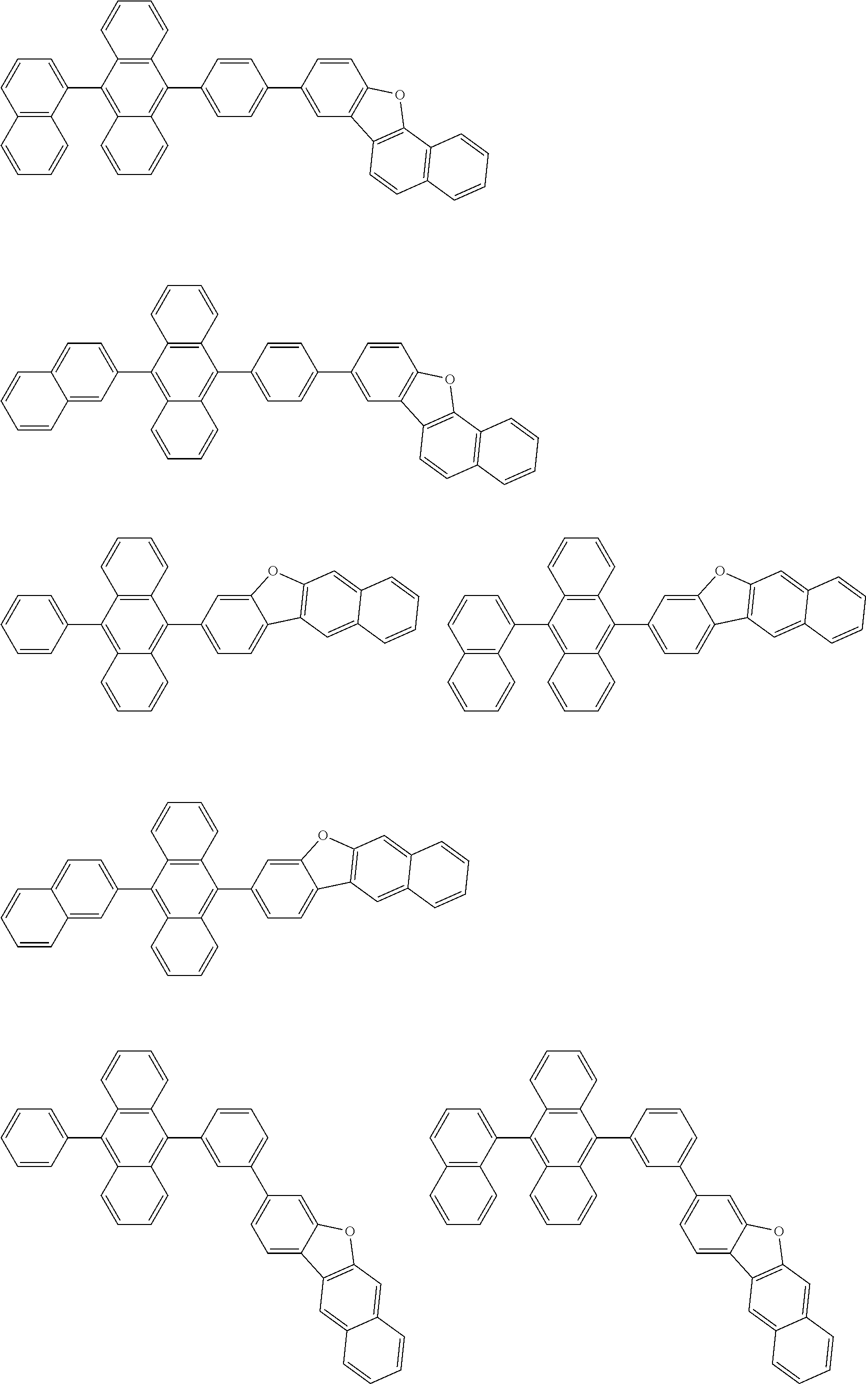

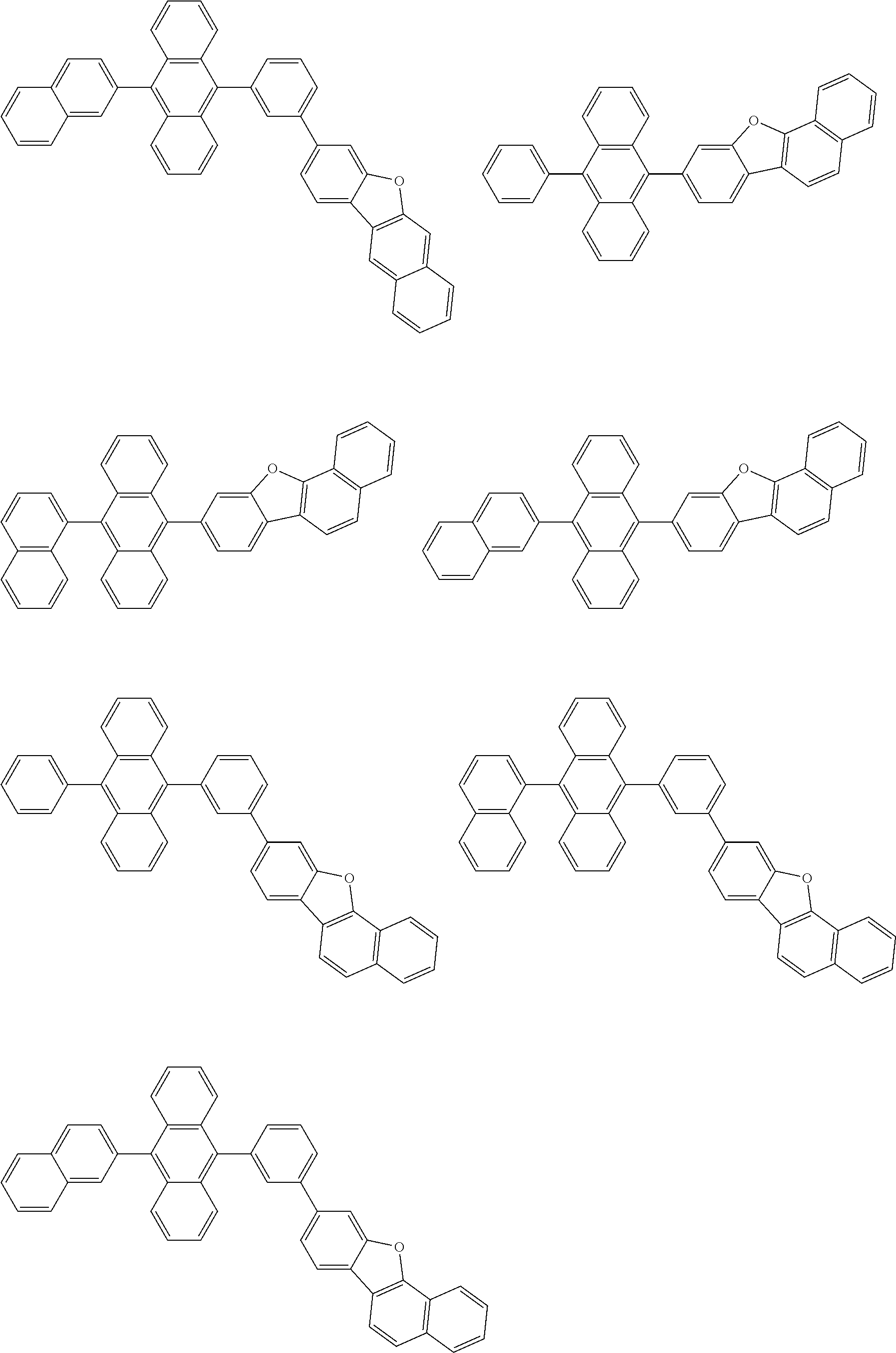

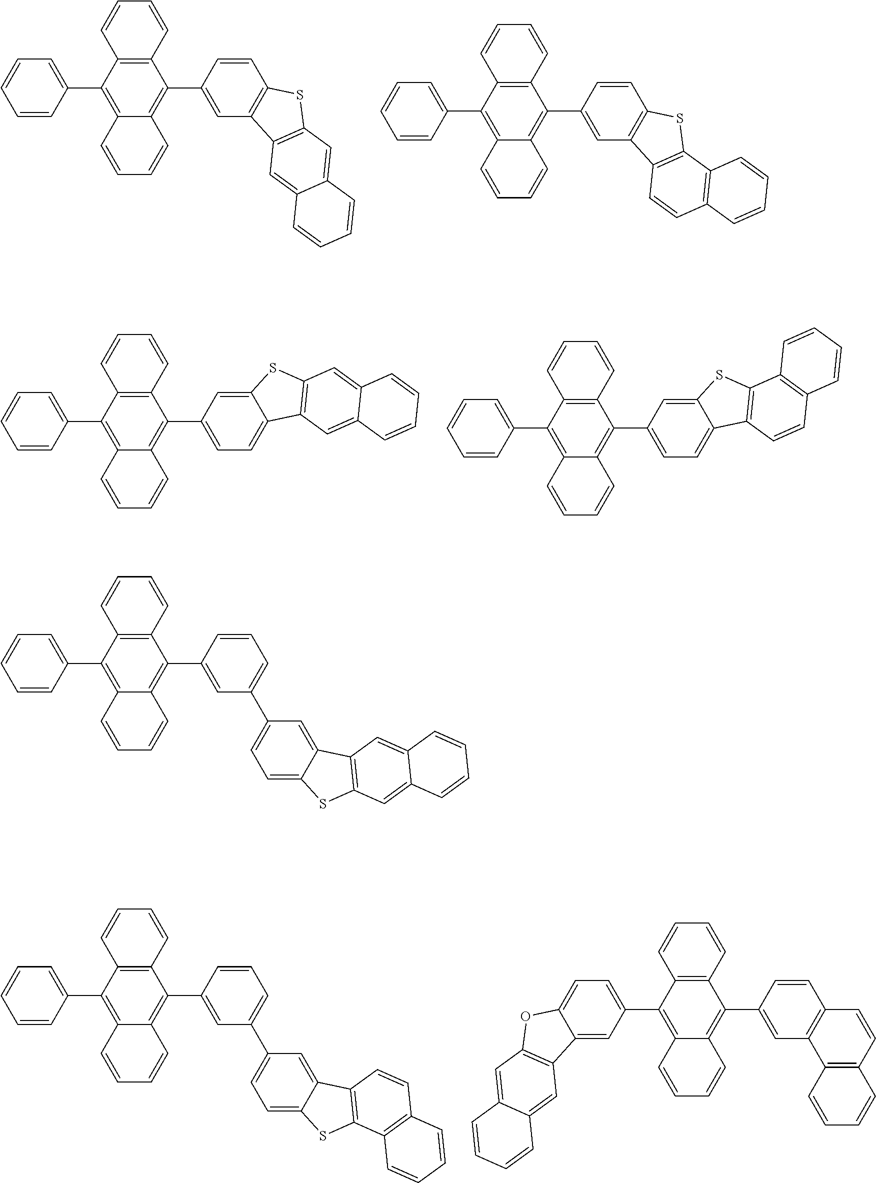

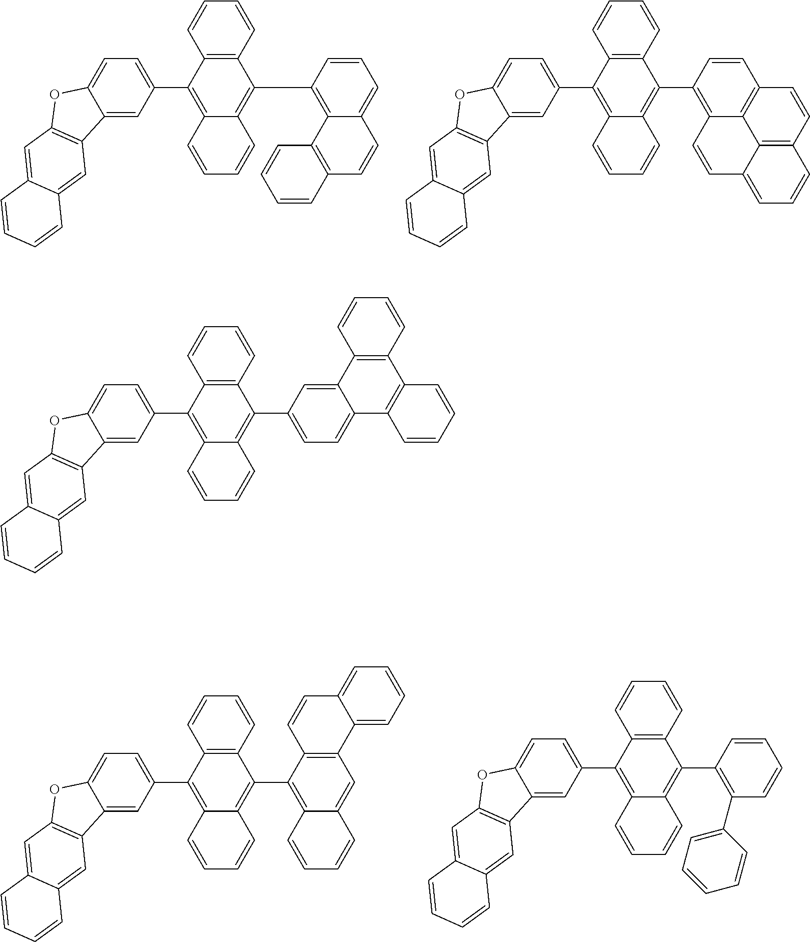 [SMILES: c1ccc2cc3c(cc2c1)oc1ccc(-c2c4ccccc4c(-c4cccc5ccc6ccccc6c45)c4ccccc24)cc13 ; c1ccc2cc3c(cc2c1)oc1ccc(-c2c4ccccc4c(-c4ccc5ccc6cccc7ccc4c5c67)c4ccccc24)cc13 ; c1ccc2cc3c(cc2c1)oc1ccc(-c2c4ccccc4c(-c4ccc5c6ccccc6c6ccccc6c5c4)c4ccccc24)cc13 ; c1ccc2cc3c(cc2c1)oc1ccc(-c2c4ccccc4c(-c4c5ccccc5cc5c4ccc4ccccc45)c4ccccc24)cc13 ; c1ccc(-c2ccccc2-c2c3ccccc3c(-c3ccc4oc5cc6ccccc6cc5c4c3)c3ccccc23)cc1]

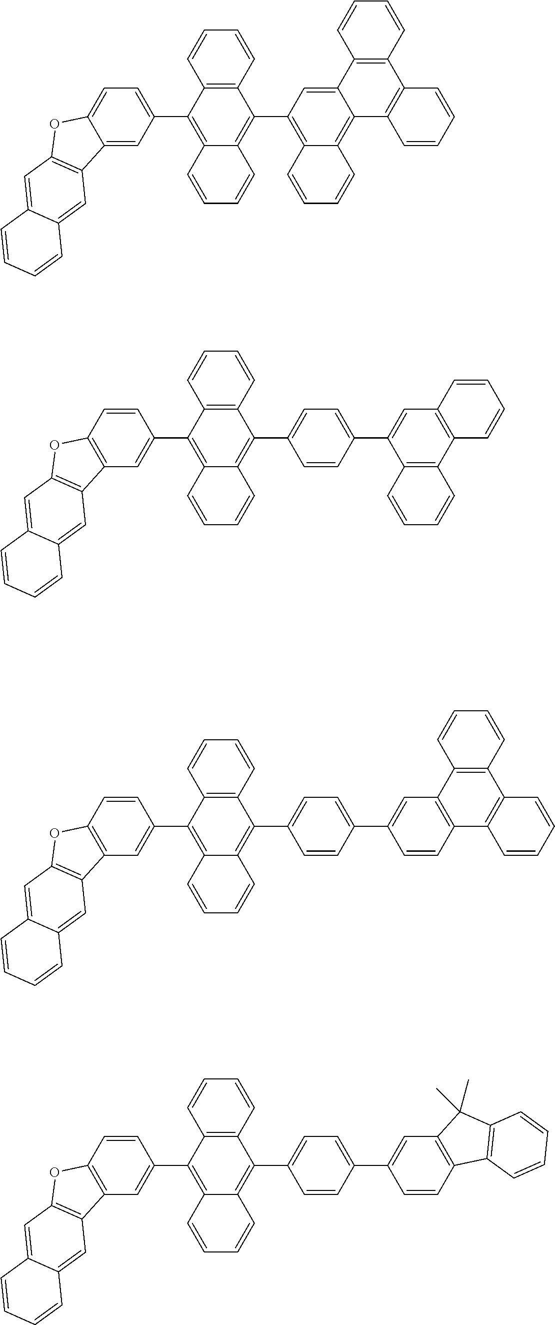 [SMILES: c1ccc2cc3c(cc2c1)oc1ccc(-c2c4ccccc4c(-c4cc5c6ccccc6c6ccccc6c5c5ccccc45)c4ccccc24)cc13 ; c1ccc2cc3c(cc2c1)oc1ccc(-c2c4ccccc4c(-c4ccc(-c5cc6ccccc6c6ccccc56)cc4)c4ccccc24)cc13 ; c1ccc2cc3c(cc2c1)oc1ccc(-c2c4ccccc4c(-c4ccc(-c5ccc6c7ccccc7c7ccccc7c6c5)cc4)c4ccccc24)cc13 ; CC1(C)c2ccccc2-c2ccc(-c3ccc(-c4c5ccccc5c(-c5ccc6oc7cc8ccccc8cc7c6c5)c5ccccc45)cc3)cc21]

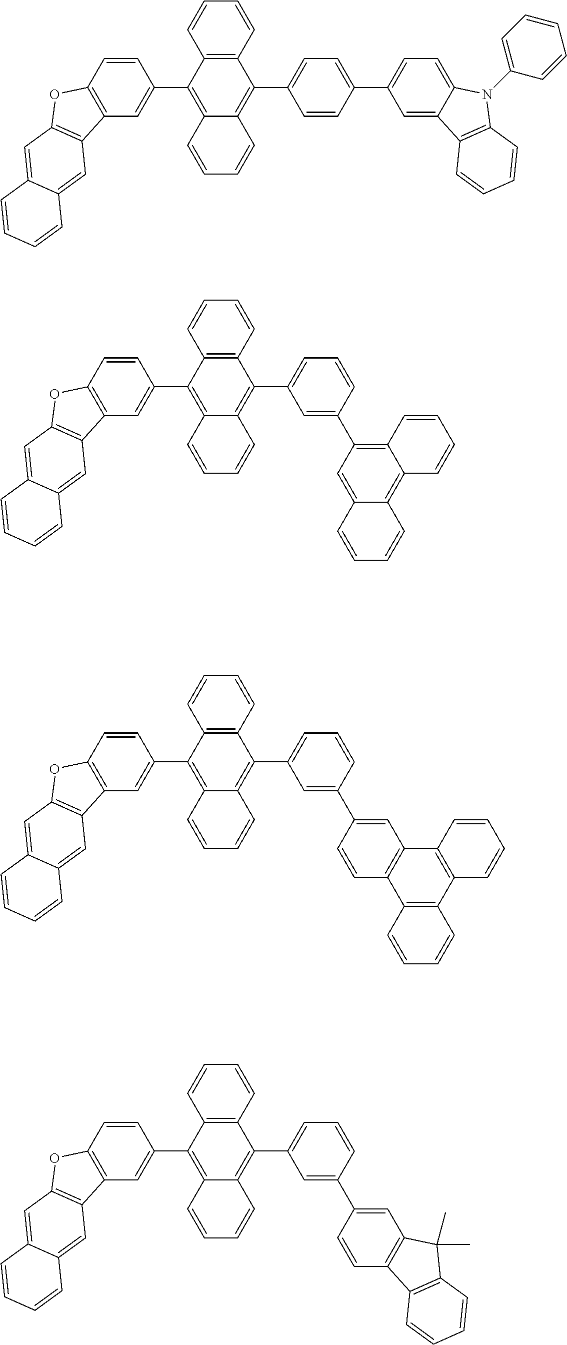 [SMILES: c1ccc(-n2c3ccccc3c3cc(-c4ccc(-c5c6ccccc6c(-c6ccc7oc8cc9ccccc9cc8c7c6)c6ccccc56)cc4)ccc32)cc1 ; c1cc(-c2c3ccccc3c(-c3ccc4oc5cc6ccccc6cc5c4c3)c3ccccc23)cc(-c2cc3ccccc3c3ccccc23)c1 ; c1cc(-c2ccc3c4ccccc4c4ccccc4c3c2)cc(-c2c3ccccc3c(-c3ccc4oc5cc6ccccc6cc5c4c3)c3ccccc23)c1 ; CC1(C)c2ccccc2-c2ccc(-c3cccc(-c4c5ccccc5c(-c5ccc6oc7cc8ccccc8cc7c6c5)c5ccccc45)c3)cc21]

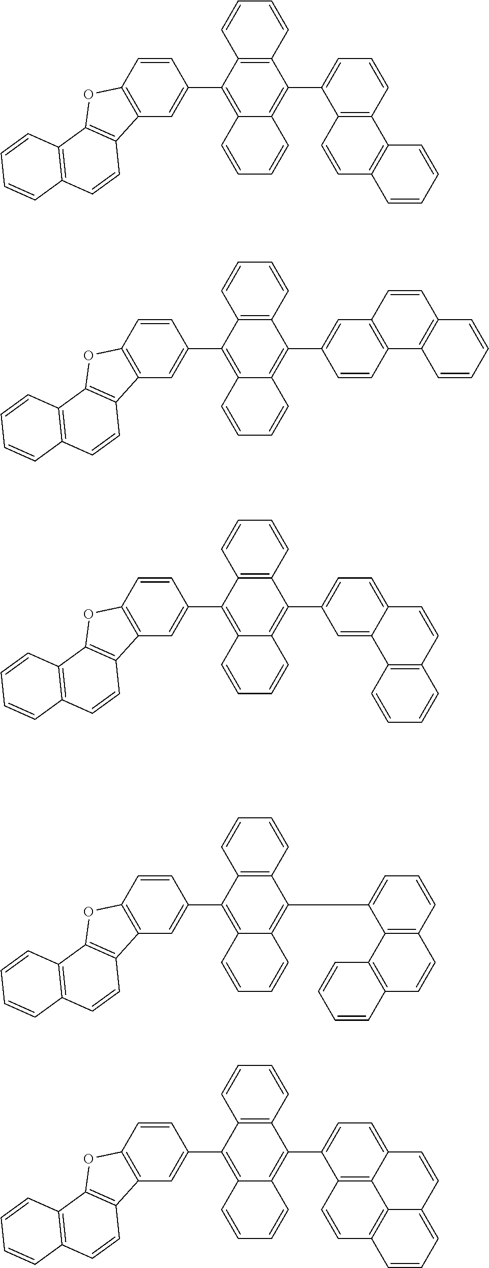 [SMILES: c1ccc2c(c1)ccc1c(-c3c4ccccc4c(-c4ccc5oc6c7ccccc7ccc6c5c4)c4ccccc34)cccc12 ; c1ccc2c(c1)ccc1cc(-c3c4ccccc4c(-c4ccc5oc6c7ccccc7ccc6c5c4)c4ccccc34)ccc12 ; c1ccc2c(c1)ccc1ccc(-c3c4ccccc4c(-c4ccc5oc6c7ccccc7ccc6c5c4)c4ccccc34)cc12 ; c1ccc2c(c1)ccc1c3cc(-c4c5ccccc5c(-c5cccc6ccc7ccccc7c56)c5ccccc45)ccc3oc21 ; c1ccc2c(c1)ccc1c3cc(-c4c5ccccc5c(-c5ccc6ccc7cccc8ccc5c6c78)c5ccccc45)ccc3oc21]

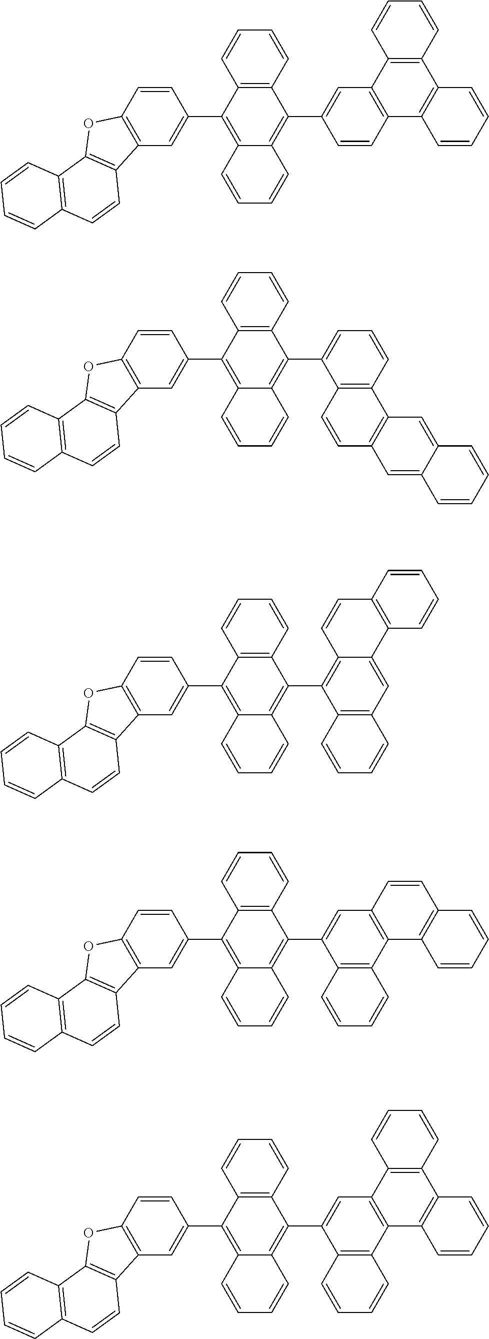 [SMILES: c1ccc2c(c1)ccc1c3cc(-c4c5ccccc5c(-c5ccc6c7ccccc7c7ccccc7c6c5)c5ccccc45)ccc3oc21 ; c1ccc2cc3c(ccc4c(-c5c6ccccc6c(-c6ccc7oc8c9ccccc9ccc8c7c6)c6ccccc56)cccc43)cc2c1 ; c1ccc2c(-c3c4ccccc4c(-c4ccc5oc6c7ccccc7ccc6c5c4)c4ccccc34)c3ccc4ccccc4c3cc2c1 ; c1ccc2c(c1)ccc1c3cc(-c4c5ccccc5c(-c5cc6ccc7ccccc7c6c6ccccc56)c5ccccc45)ccc3oc21 ; c1ccc2c(c1)ccc1c3cc(-c4c5ccccc5c(-c5cc6c7ccccc7c7ccccc7c6c6ccccc56)c5ccccc45)ccc3oc21]

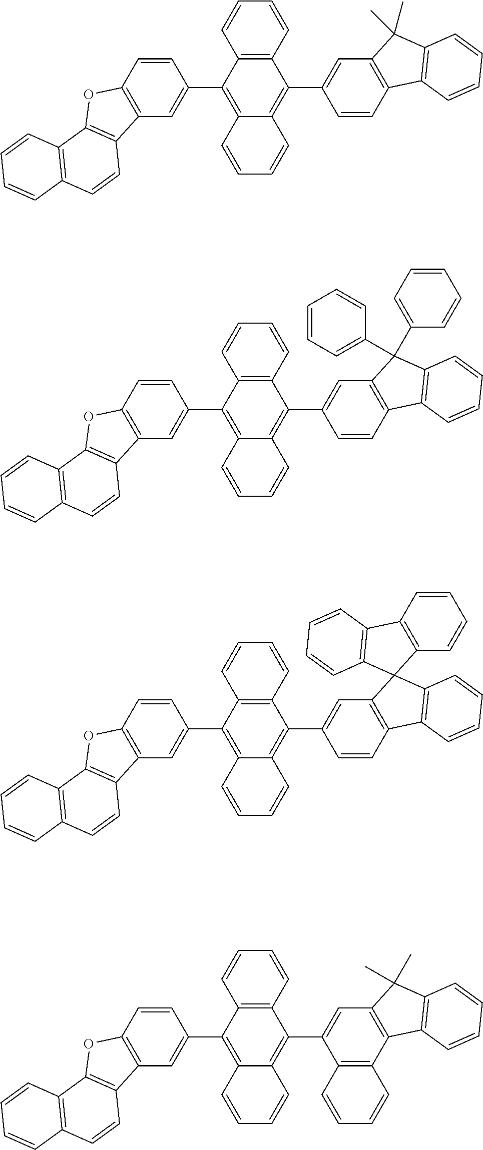 [SMILES: CC1(C)c2ccccc2-c2ccc(-c3c4ccccc4c(-c4ccc5oc6c7ccccc7ccc6c5c4)c4ccccc34)cc21 ; c1ccc(C2(c3ccccc3)c3ccccc3-c3ccc(-c4c5ccccc5c(-c5ccc6oc7c8ccccc8ccc7c6c5)c5ccccc45)cc32)cc1 ; c1ccc2c(c1)-c1ccccc1C21c2ccccc2-c2ccc(-c3c4ccccc4c(-c4ccc5oc6c7ccccc7ccc6c5c4)c4ccccc34)cc21 ; CC1(C)c2ccccc2-c2c1cc(-c1c3ccccc3c(-c3ccc4oc5c6ccccc6ccc5c4c3)c3ccccc13)c1ccccc21]

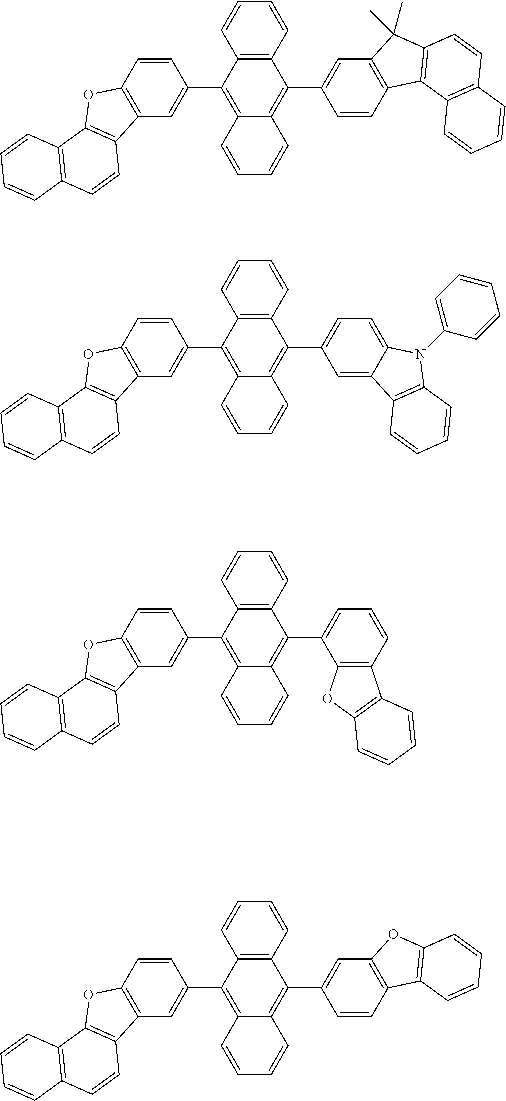 [SMILES: CC1(C)c2cc(-c3c4ccccc4c(-c4ccc5oc6c7ccccc7ccc6c5c4)c4ccccc34)ccc2-c2c1ccc1ccccc21 ; c1ccc(-n2c3ccccc3c3cc(-c4c5ccccc5c(-c5ccc6oc7c8ccccc8ccc7c6c5)c5ccccc45)ccc32)cc1 ; c1ccc2c(c1)ccc1c3cc(-c4c5ccccc5c(-c5cccc6c5oc5ccccc56)c5ccccc45)ccc3oc21 ; c1ccc2c(c1)ccc1c3cc(-c4c5ccccc5c(-c5ccc6c(c5)oc5ccccc56)c5ccccc45)ccc3oc21]

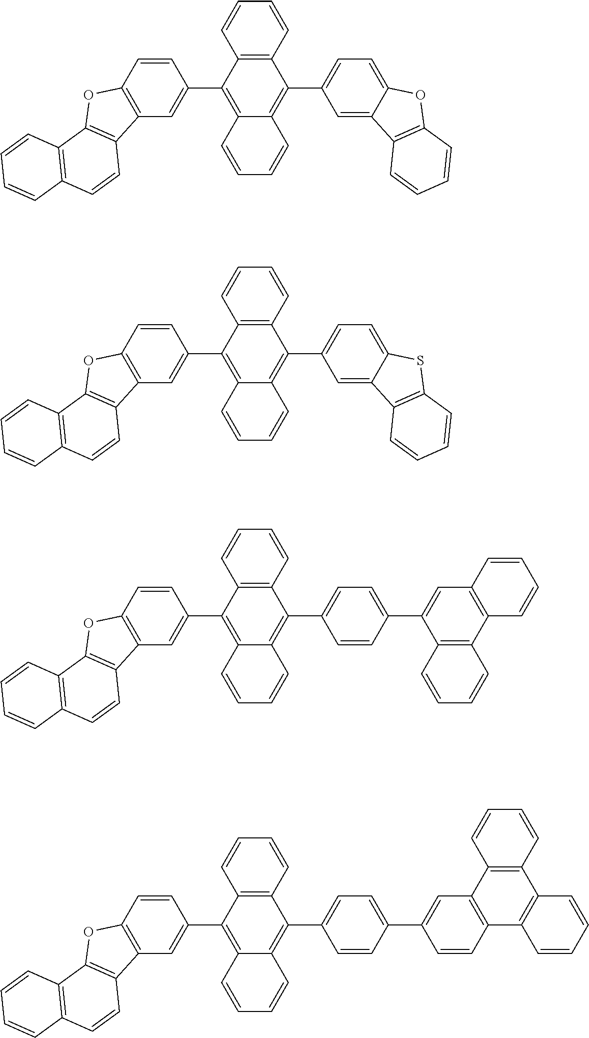 [SMILES: c1ccc2c(c1)ccc1c3cc(-c4c5ccccc5c(-c5ccc6oc7ccccc7c6c5)c5ccccc45)ccc3oc21 ; c1ccc2c(c1)ccc1c3cc(-c4c5ccccc5c(-c5ccc6sc7ccccc7c6c5)c5ccccc45)ccc3oc21 ; c1ccc2c(c1)cc(-c1ccc(-c3c4ccccc4c(-c4ccc5oc6c7ccccc7ccc6c5c4)c4ccccc34)cc1)c1ccccc12 ; c1ccc2c(c1)ccc1c3cc(-c4c5ccccc5c(-c5ccc(-c6ccc7c8ccccc8c8ccccc8c7c6)cc5)c5ccccc45)ccc3oc21]

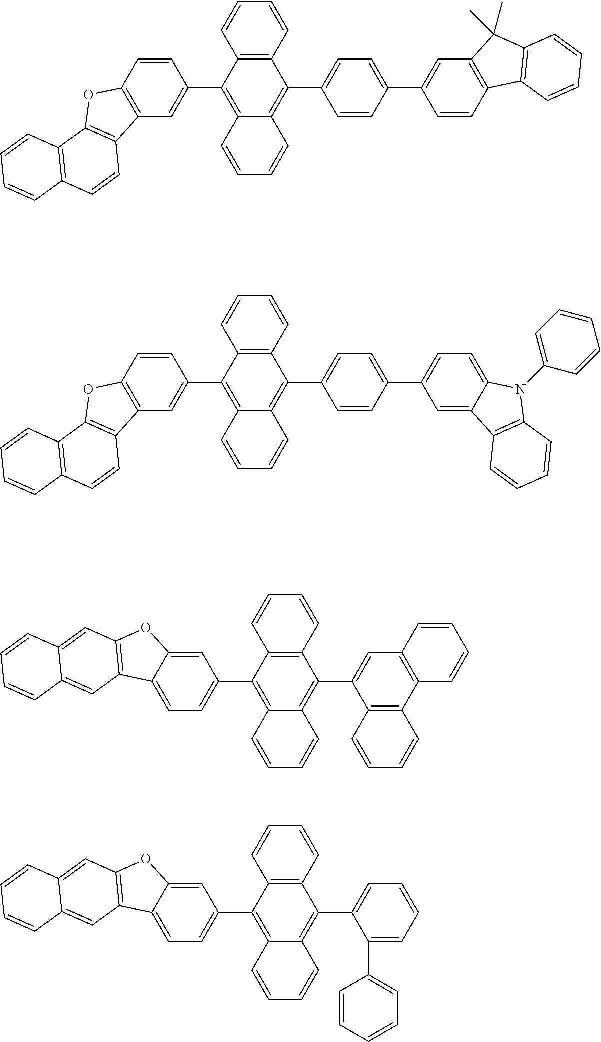 [SMILES: CC1(C)c2ccccc2-c2ccc(-c3ccc(-c4c5ccccc5c(-c5ccc6oc7c8ccccc8ccc7c6c5)c5ccccc45)cc3)cc21 ; c1ccc(-n2c3ccccc3c3cc(-c4ccc(-c5c6ccccc6c(-c6ccc7oc8c9ccccc9ccc8c7c6)c6ccccc56)cc4)ccc32)cc1 ; c1ccc2cc3c(cc2c1)oc1cc(-c2c4ccccc4c(-c4cc5ccccc5c5ccccc45)c4ccccc24)ccc13 ; c1ccc(-c2ccccc2-c2c3ccccc3c(-c3ccc4c(c3)oc3cc5ccccc5cc34)c3ccccc23)cc1]

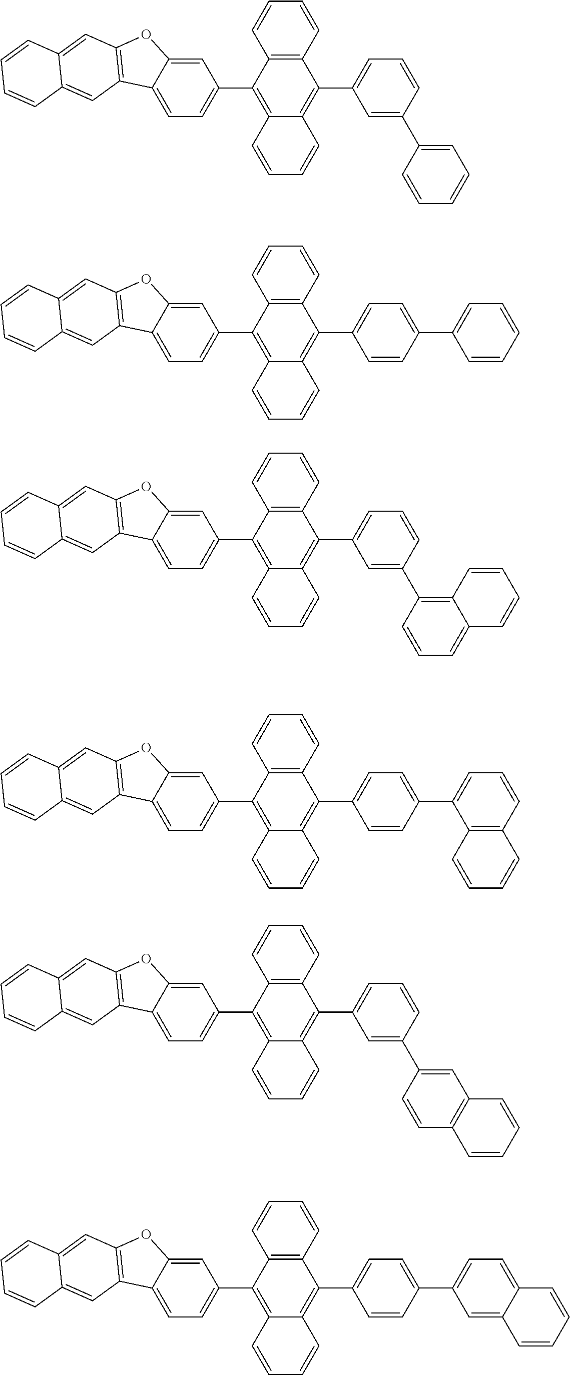

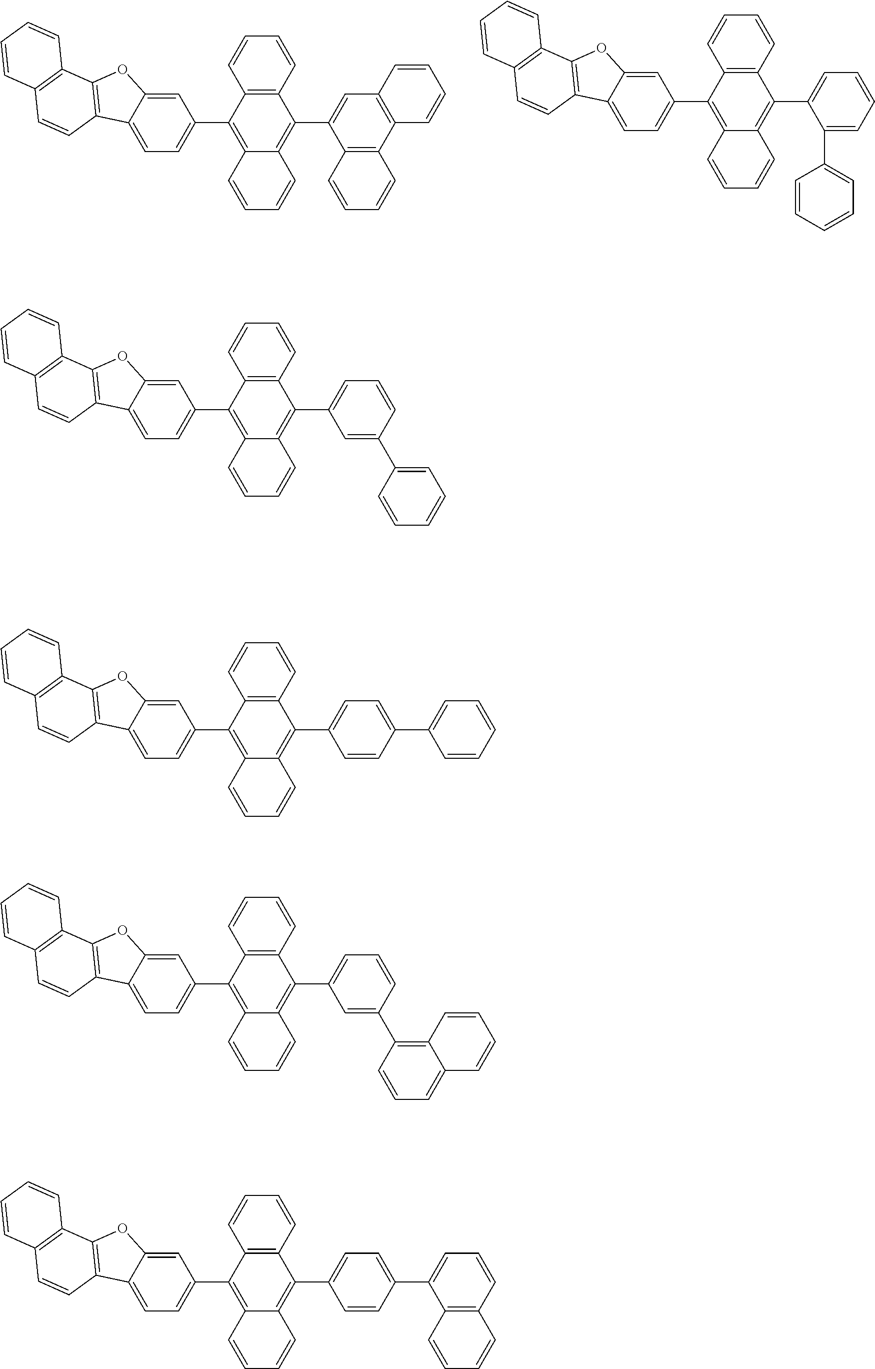 [SMILES: c1ccc2c(c1)cc(-c1c3ccccc3c(-c3ccc4c(c3)oc3c5ccccc5ccc43)c3ccccc13)c1ccccc12 ; c1ccc(-c2ccccc2-c2c3ccccc3c(-c3ccc4c(c3)oc3c5ccccc5ccc43)c3ccccc23)cc1 ; c1ccc(-c2cccc(-c3c4ccccc4c(-c4ccc5c(c4)oc4c6ccccc6ccc54)c4ccccc34)c2)cc1 ; c1ccc(-c2ccc(-c3c4ccccc4c(-c4ccc5c(c4)oc4c6ccccc6ccc54)c4ccccc34)cc2)cc1 ; c1cc(-c2cccc3ccccc23)cc(-c2c3ccccc3c(-c3ccc4c(c3)oc3c5ccccc5ccc43)c3ccccc23)c1 ; c1ccc2c(-c3ccc(-c4c5ccccc5c(-c5ccc6c(c5)oc5c7ccccc7ccc65)c5ccccc45)cc3)cccc2c1]

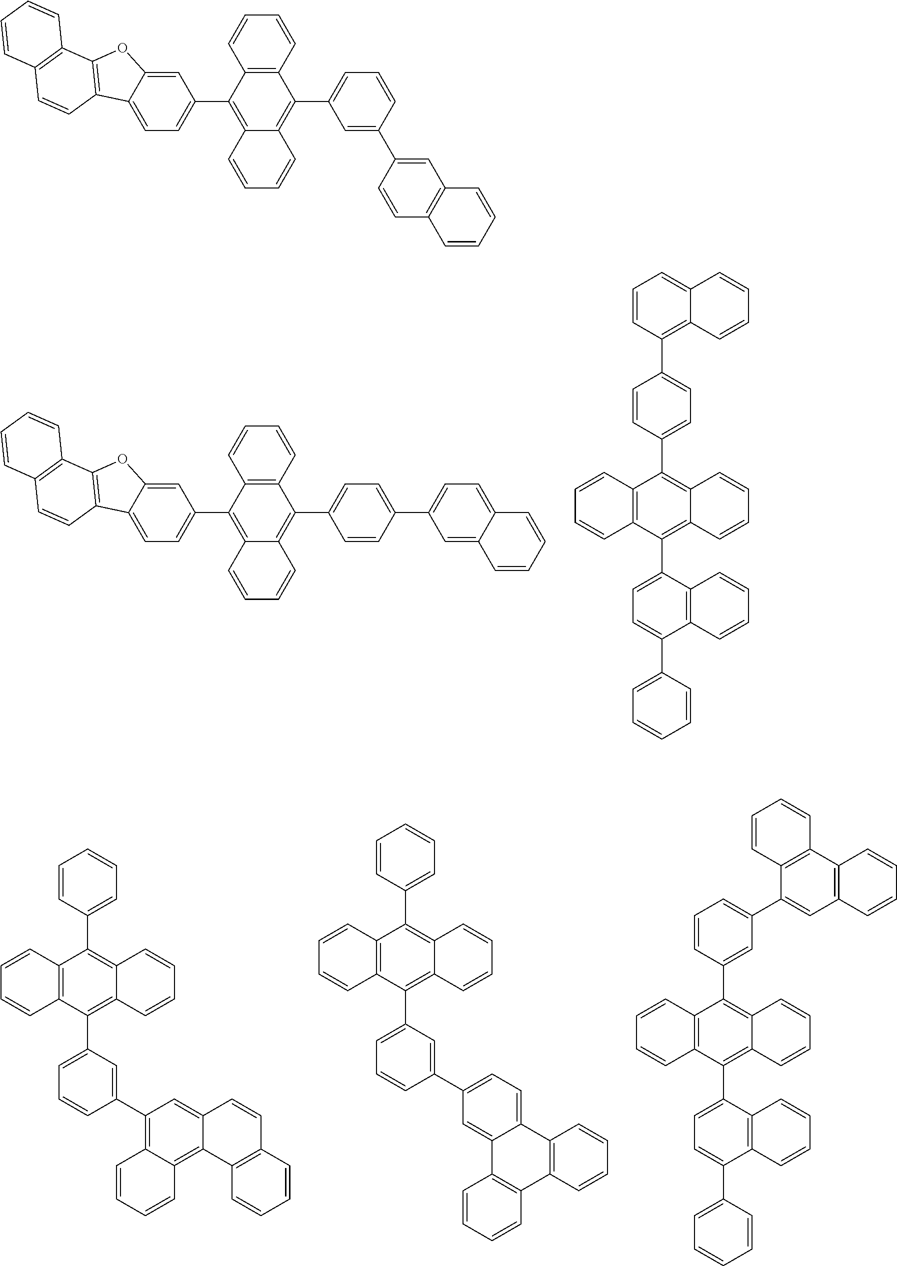

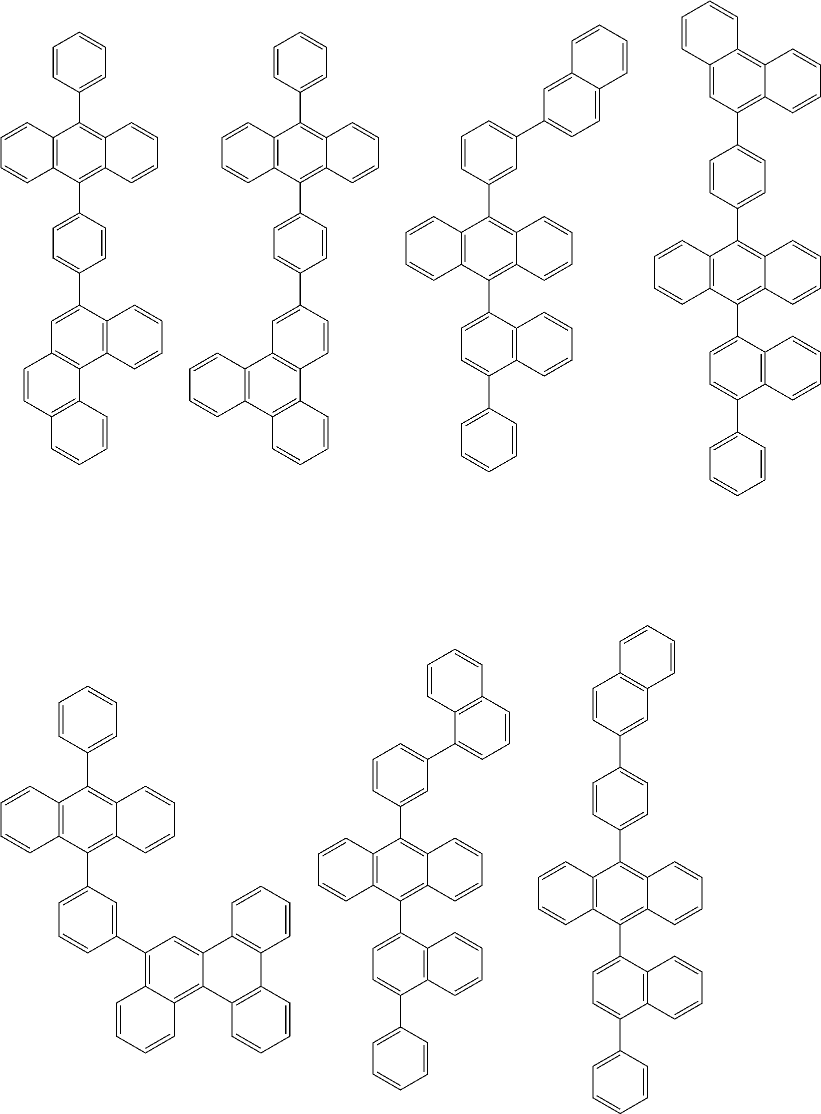

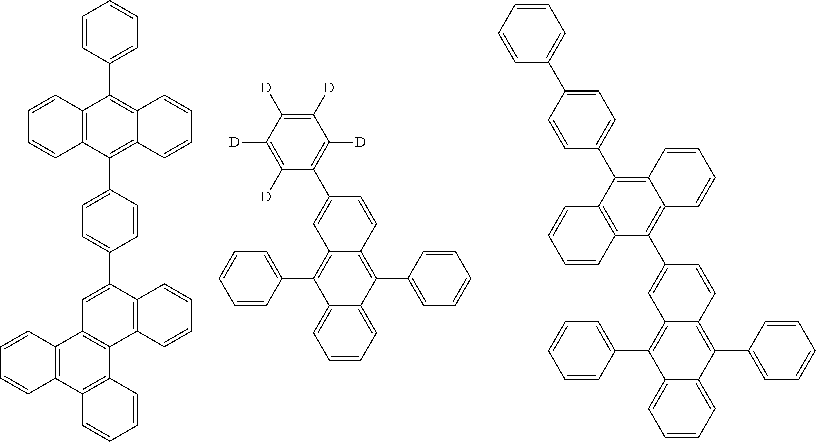

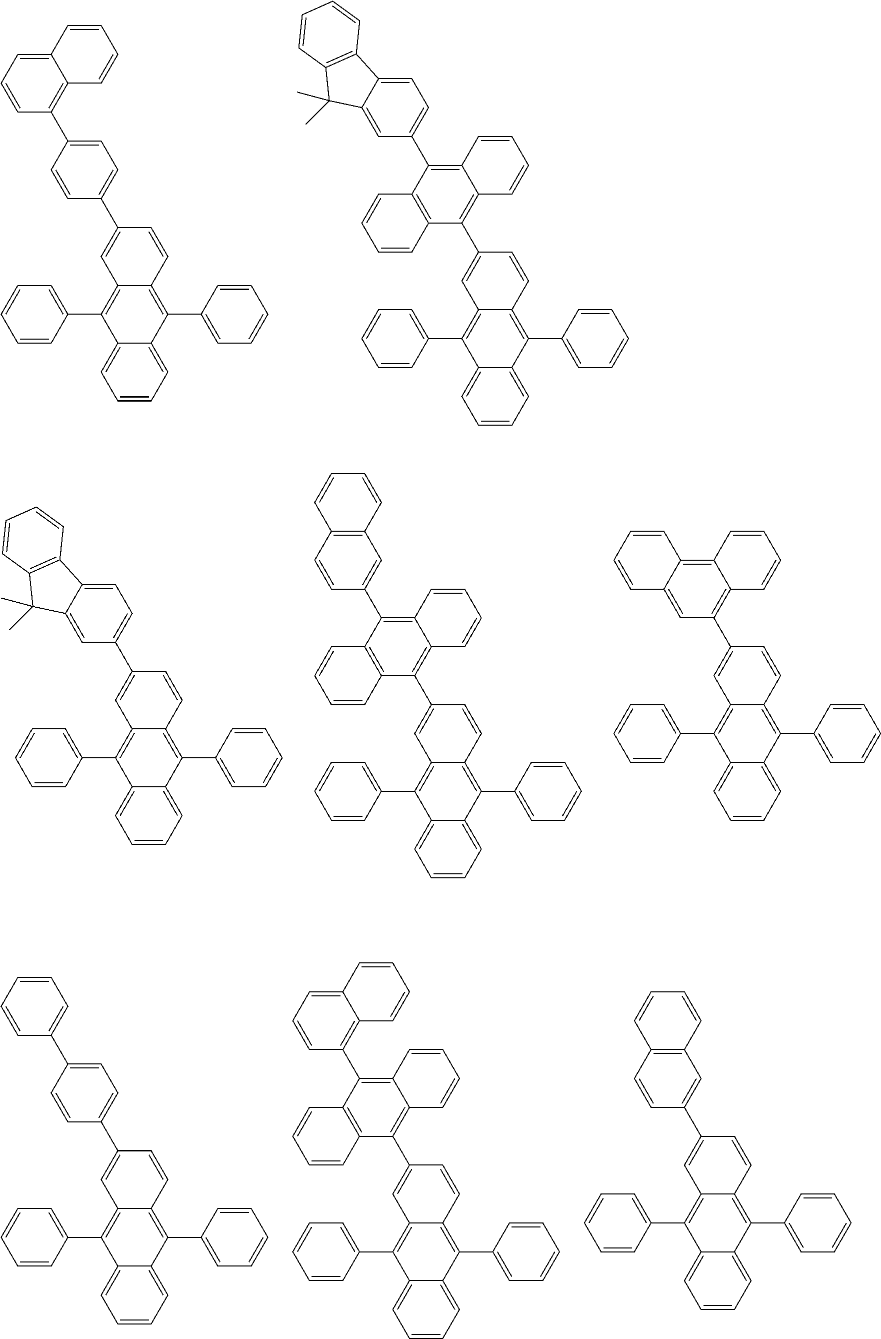

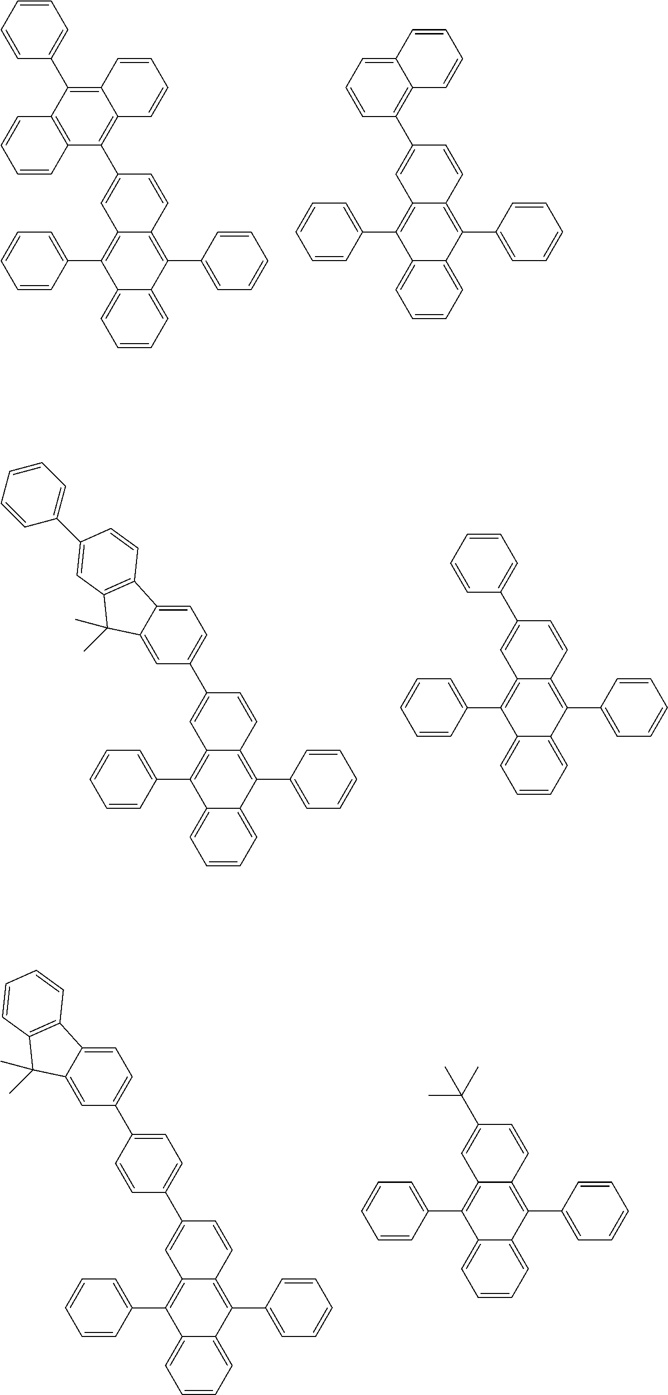

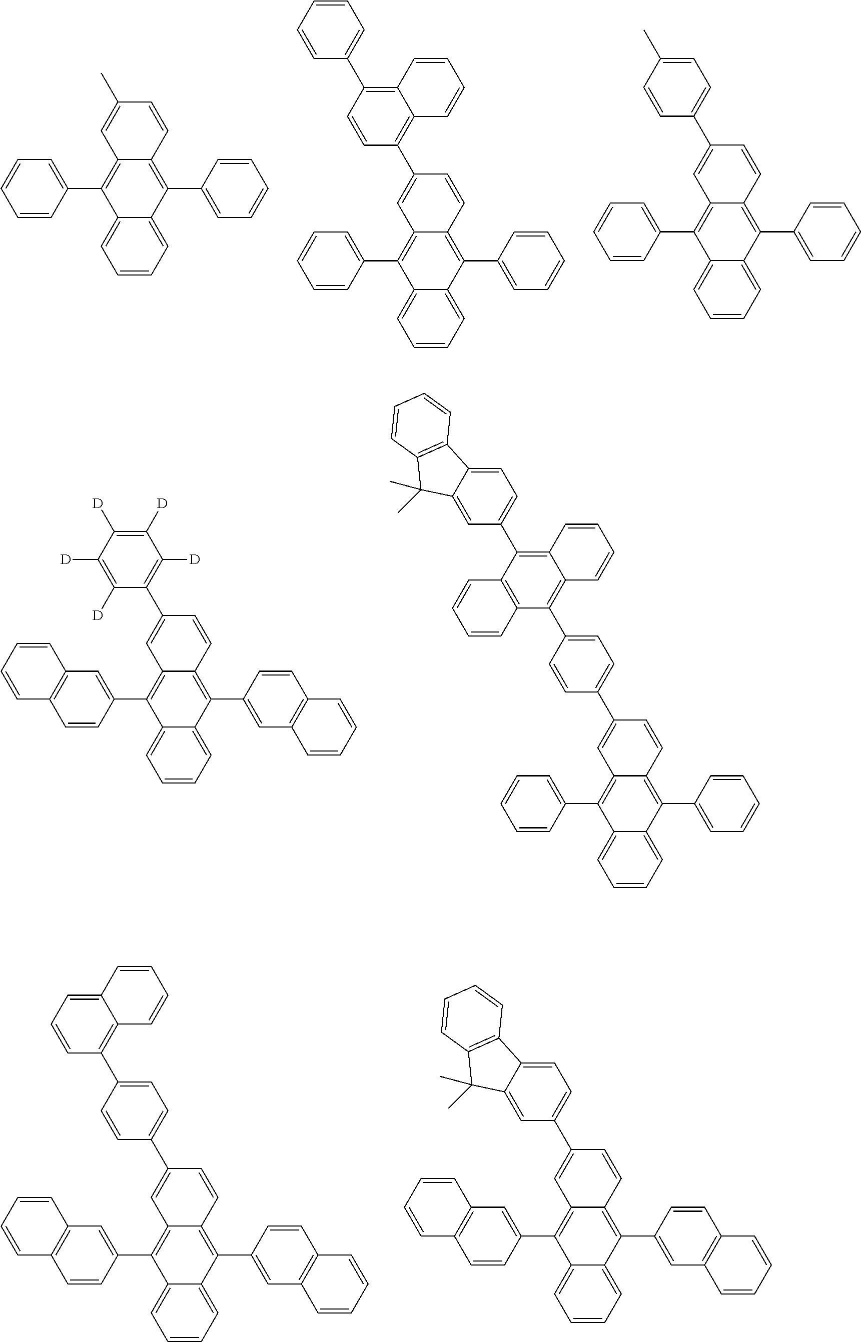

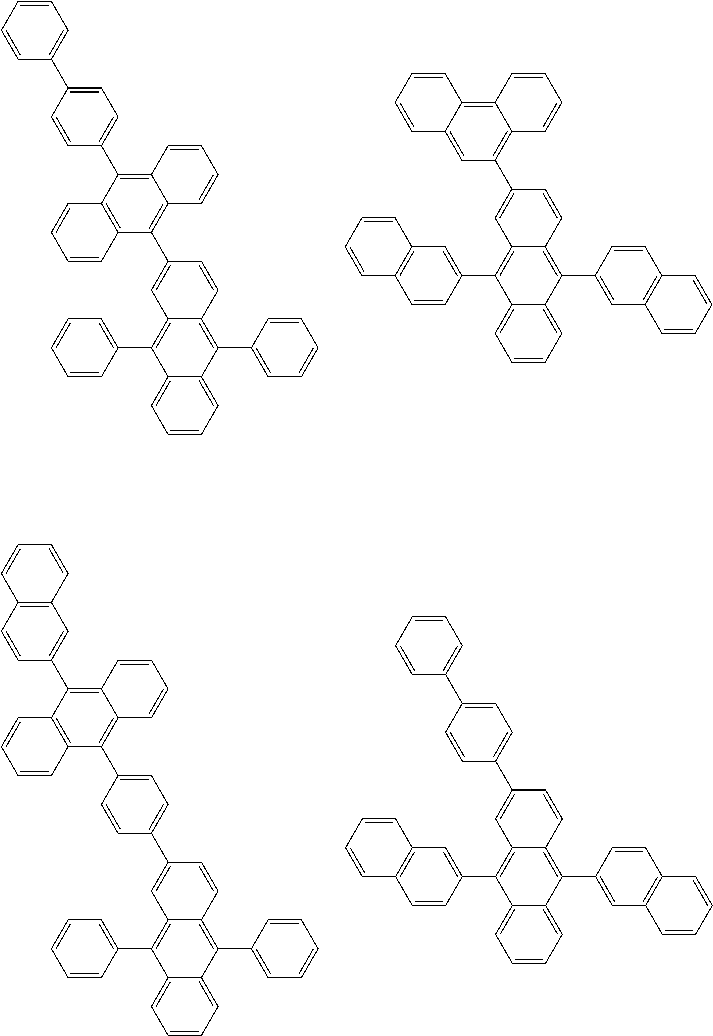

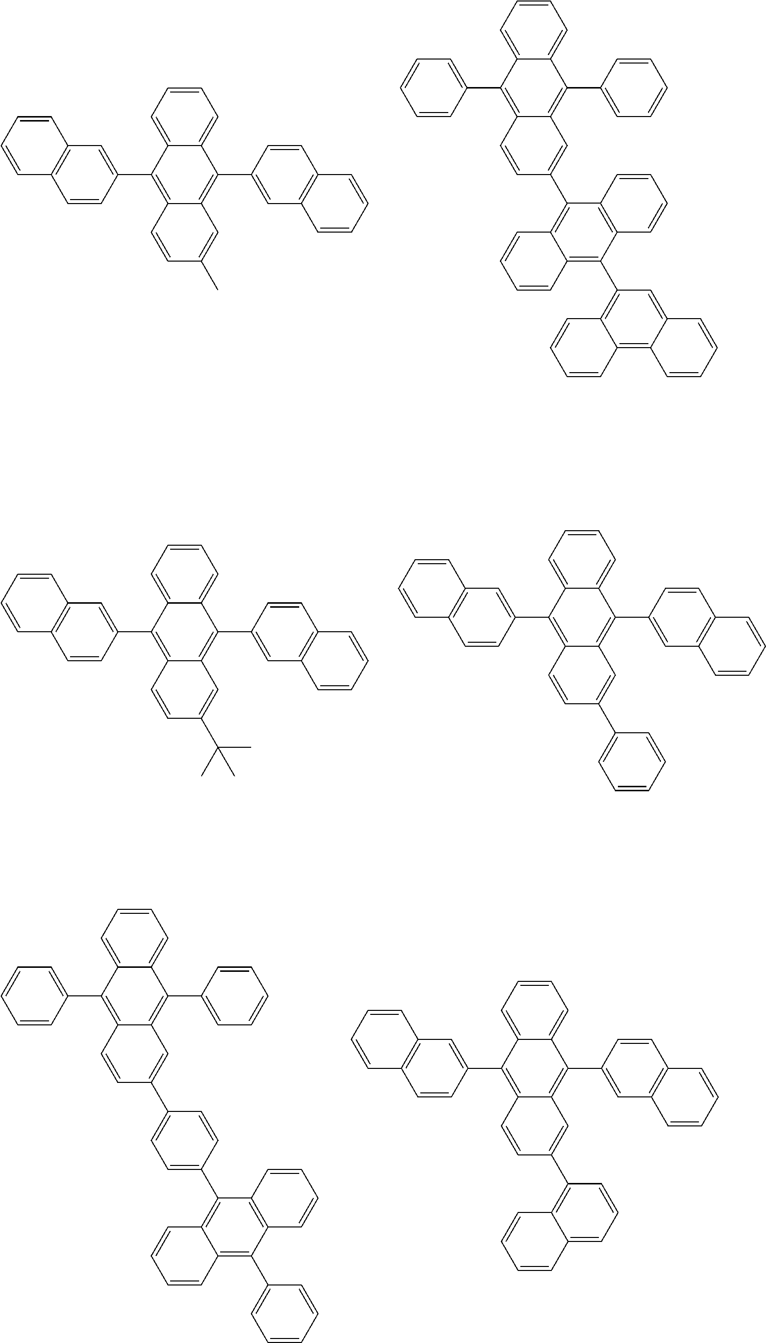

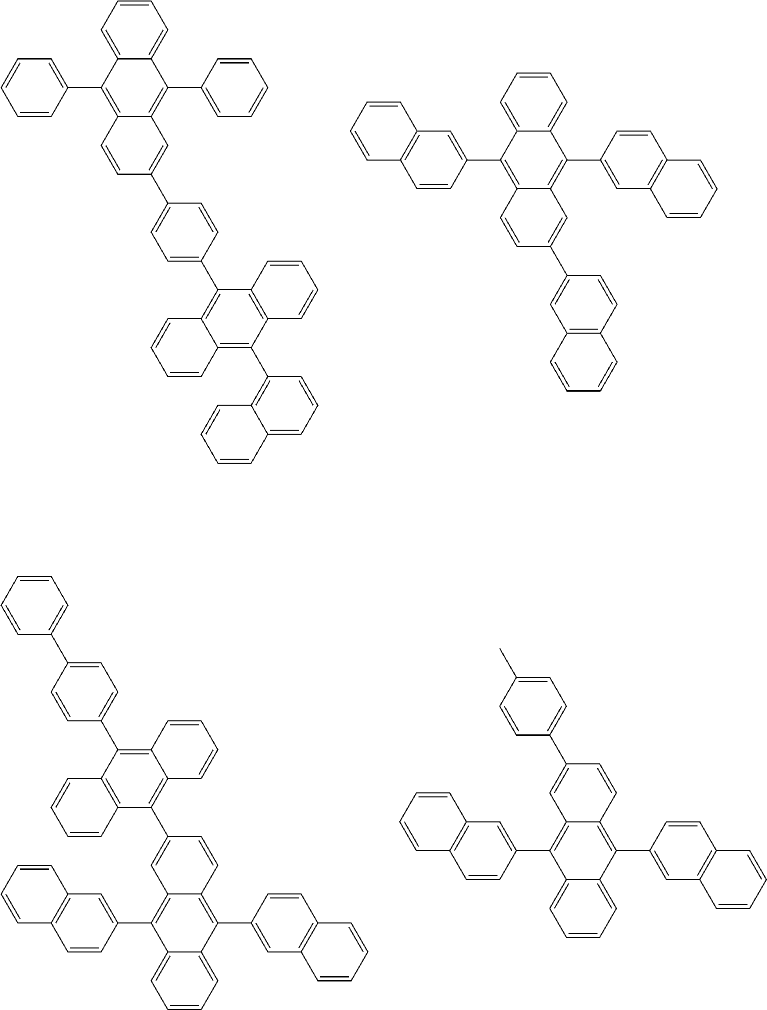

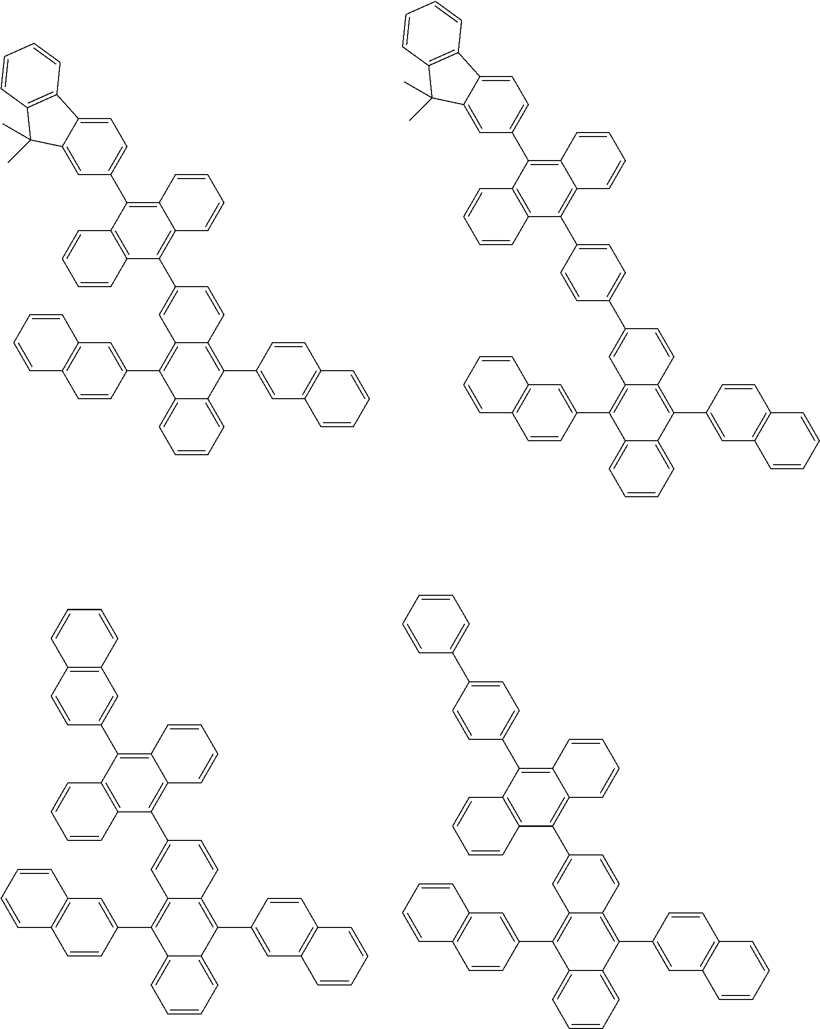

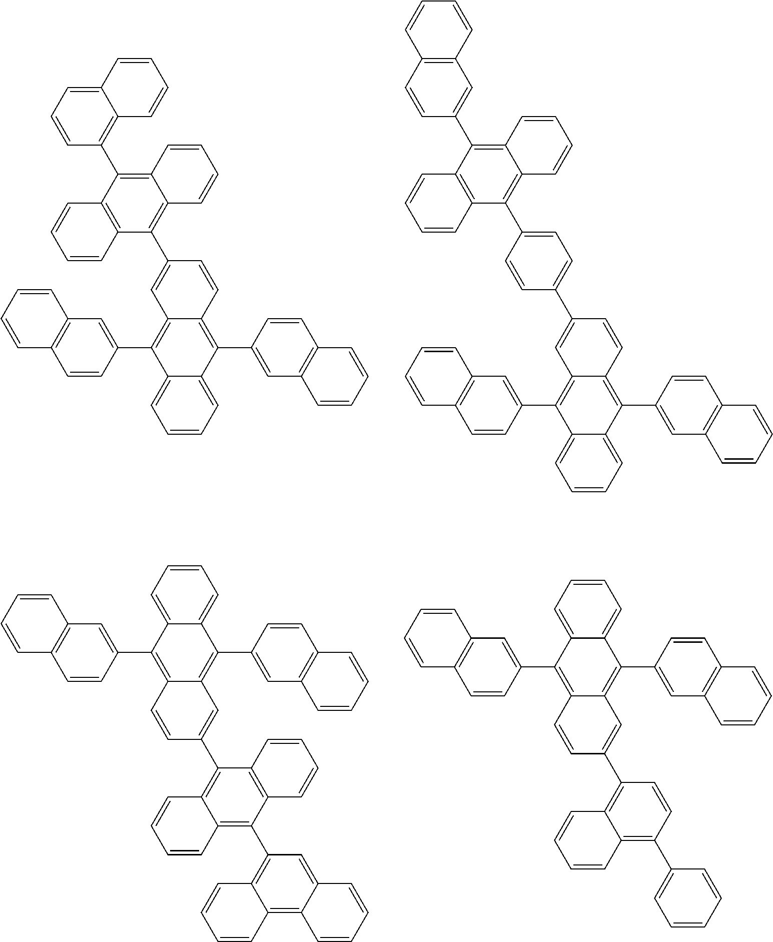

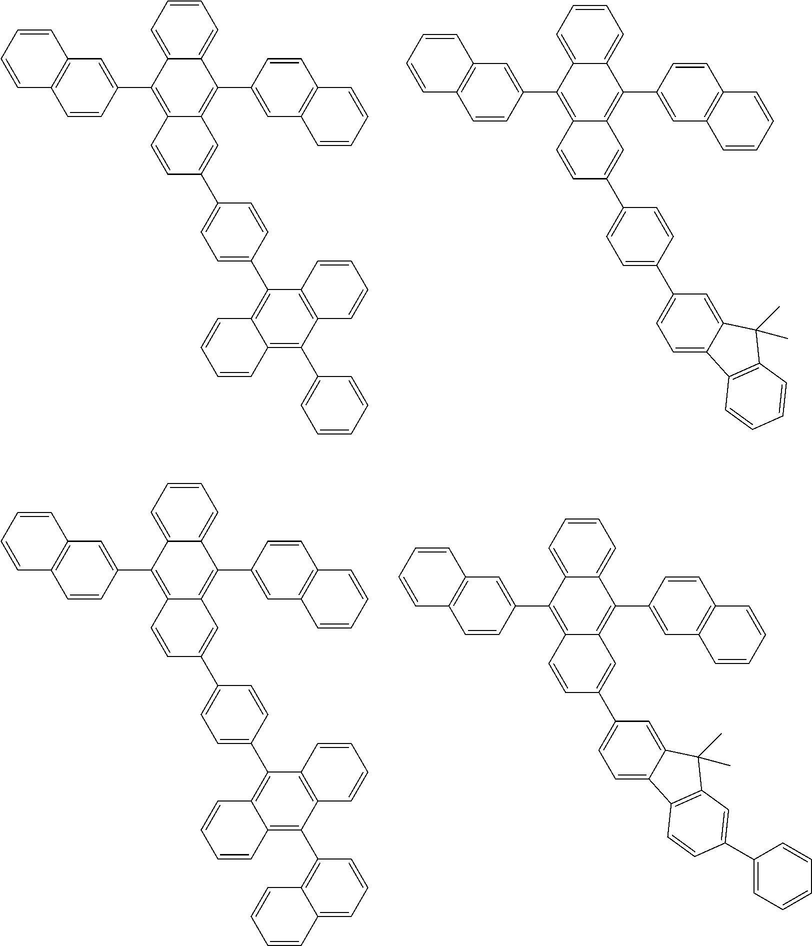

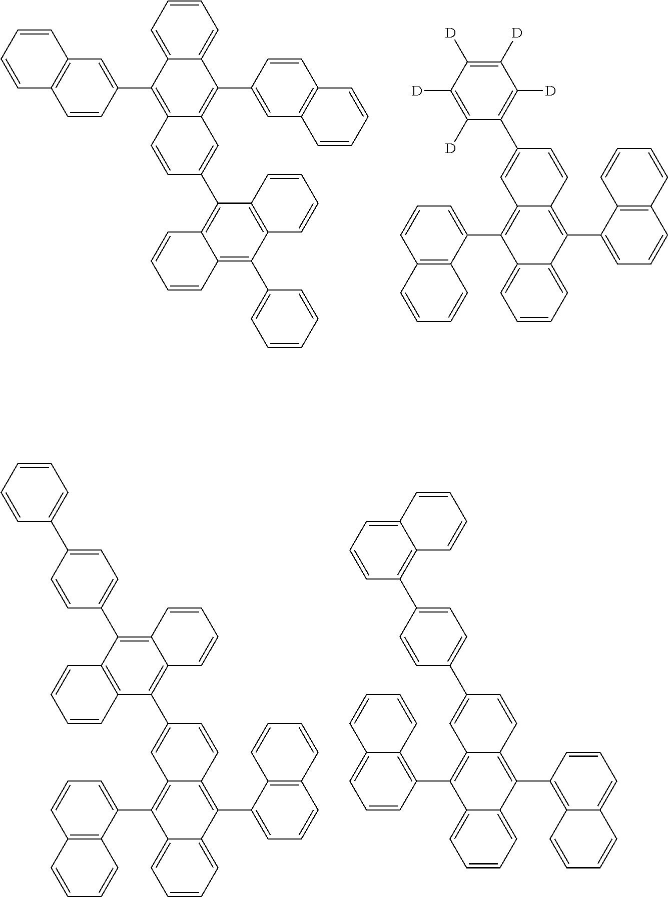

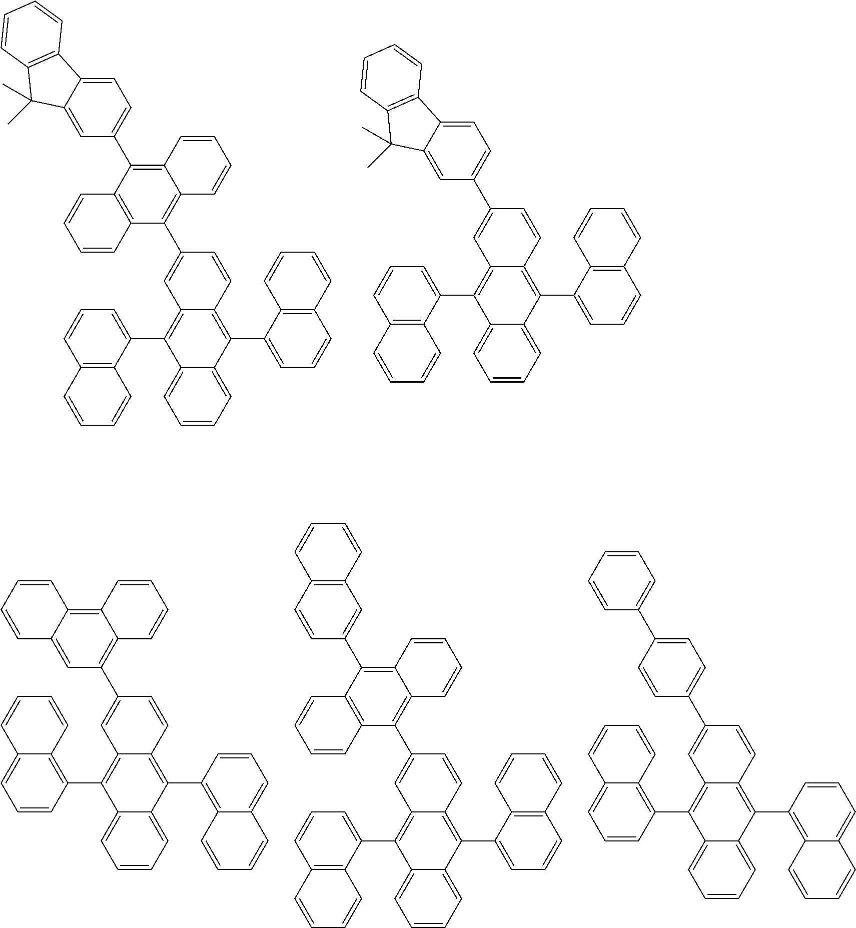

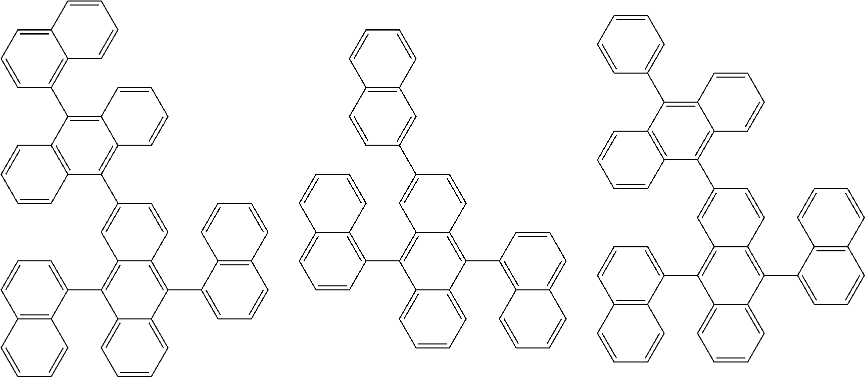

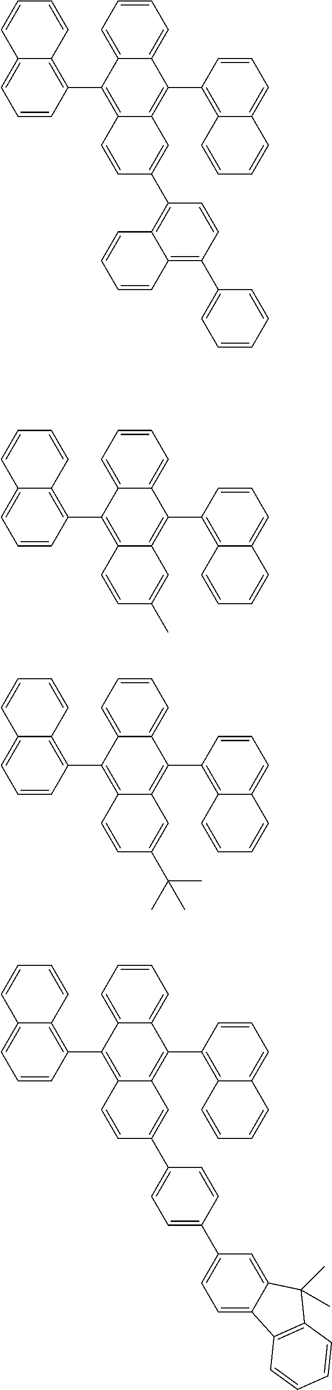

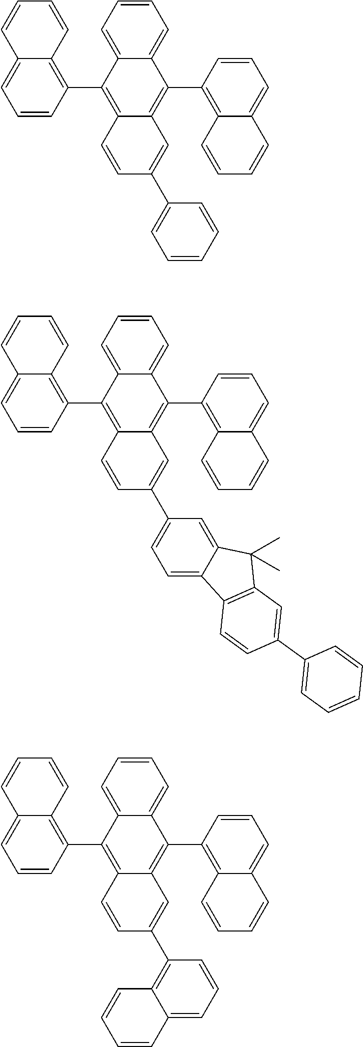

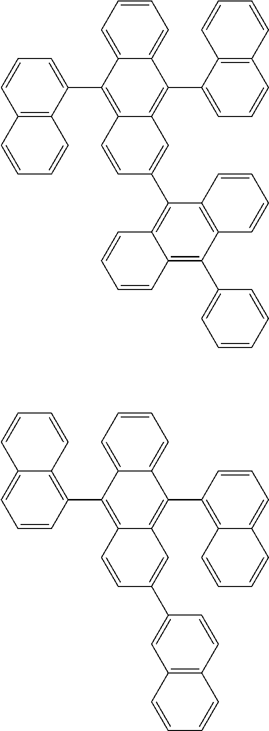

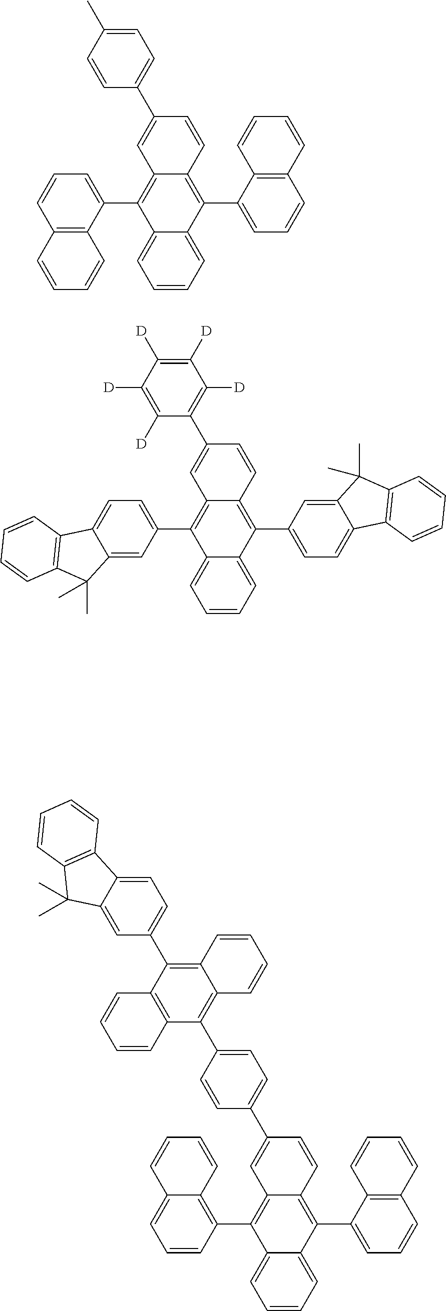

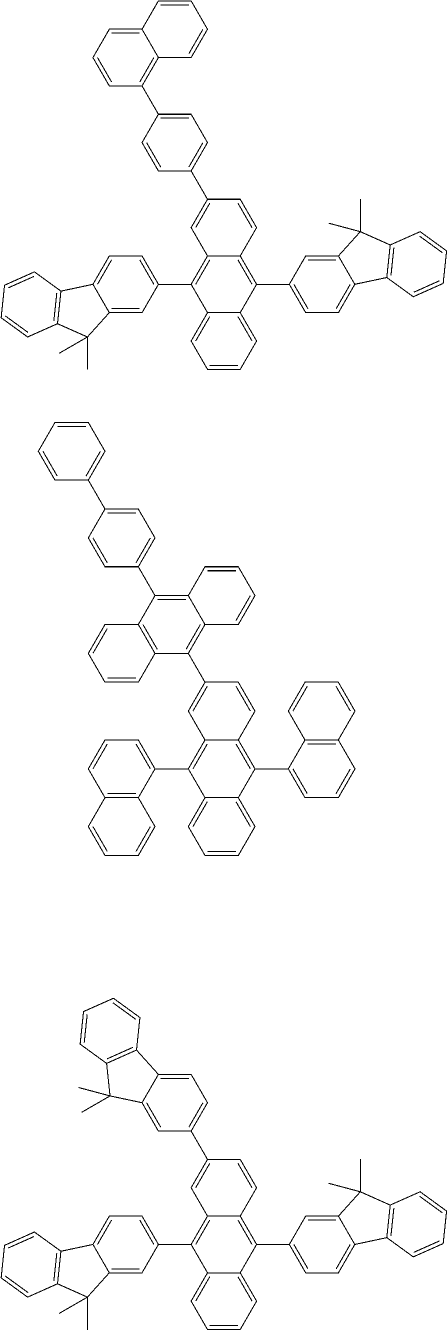

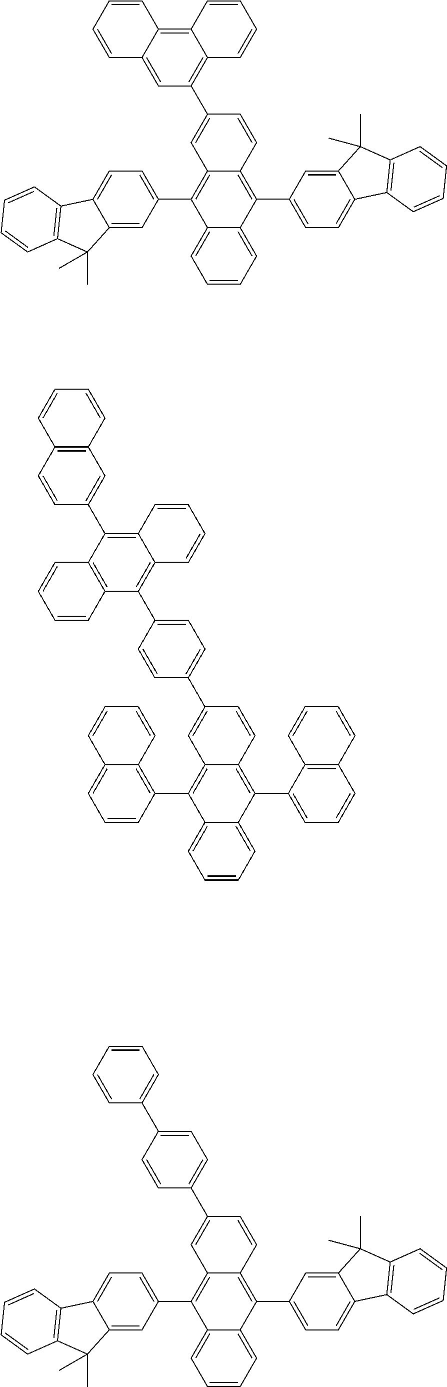

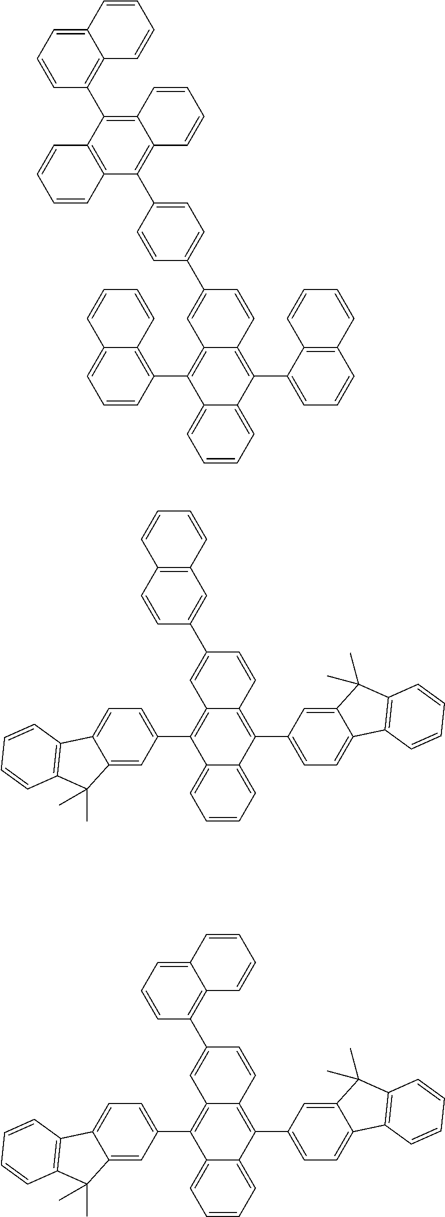

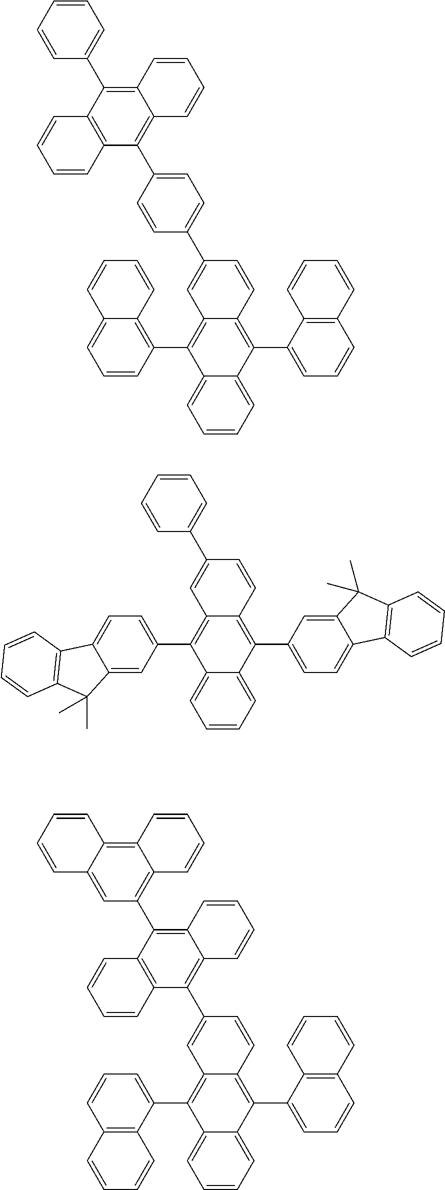

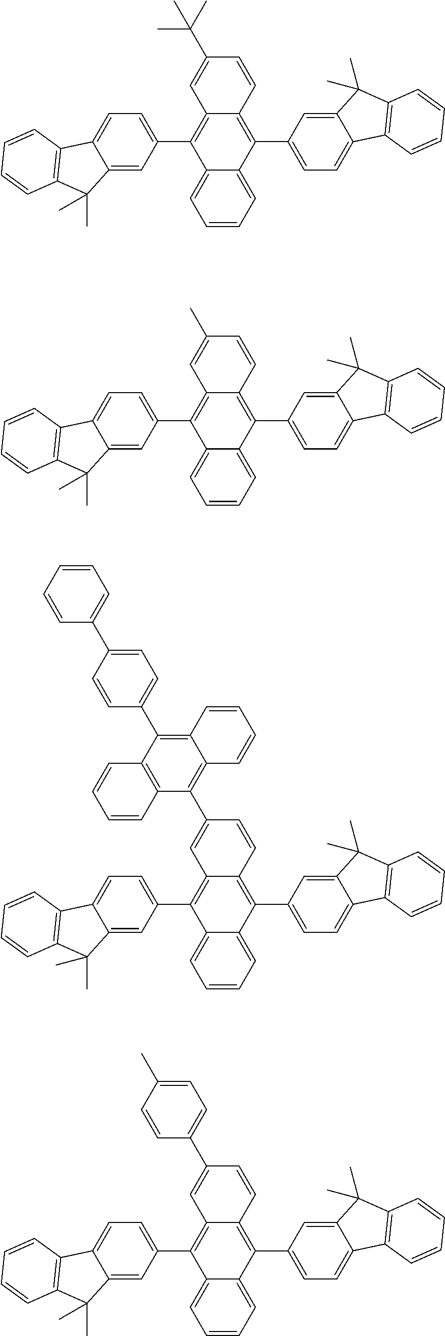

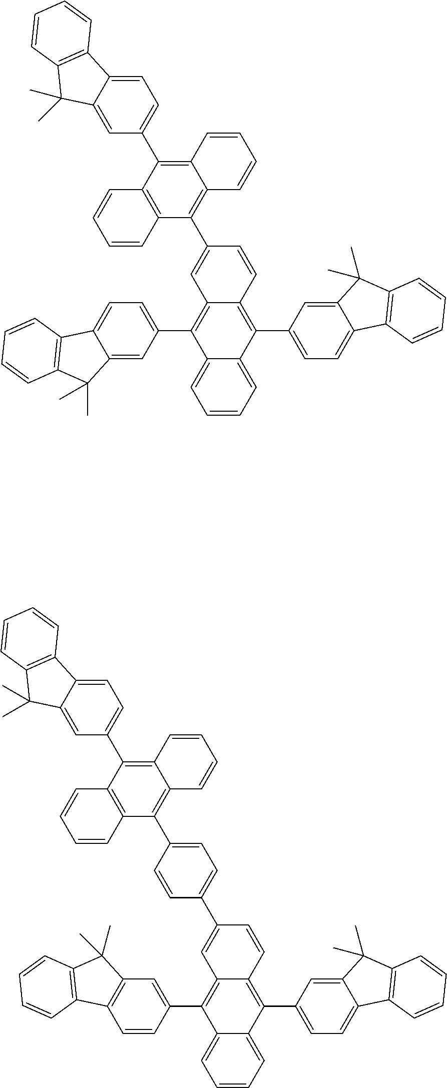

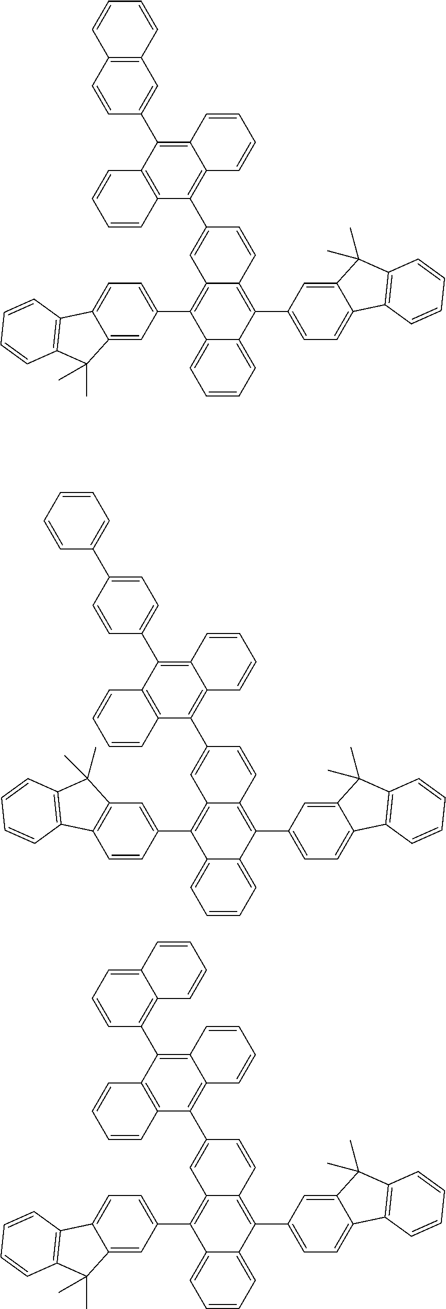

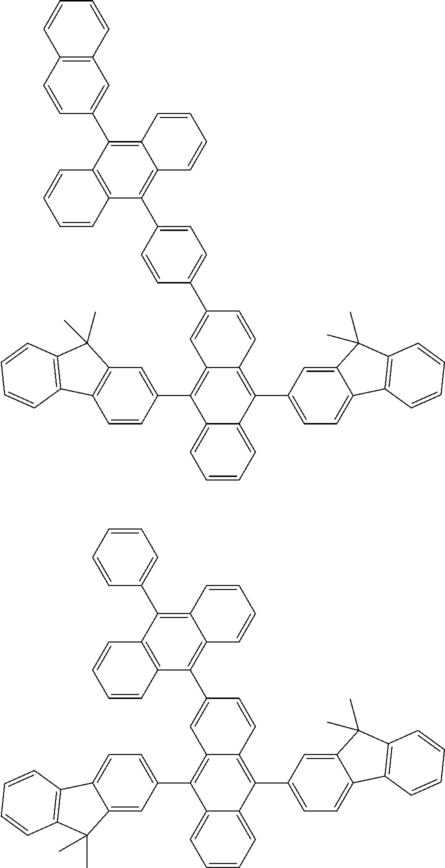

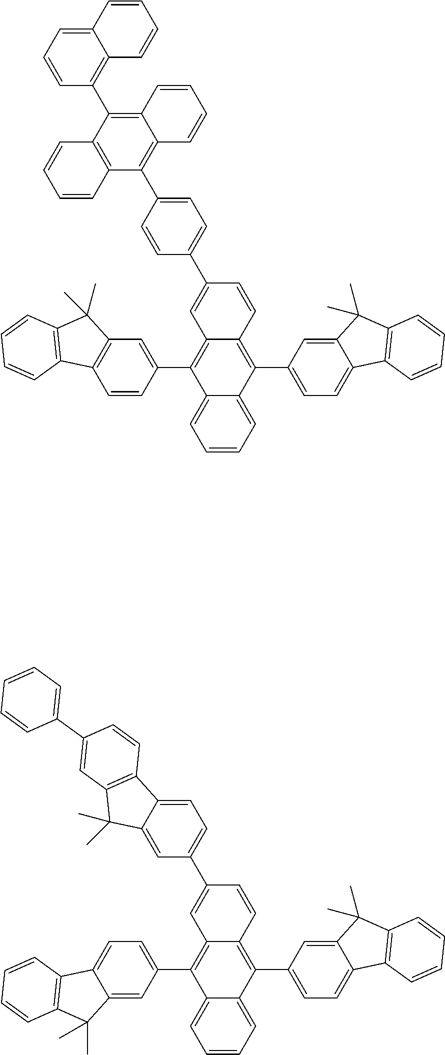

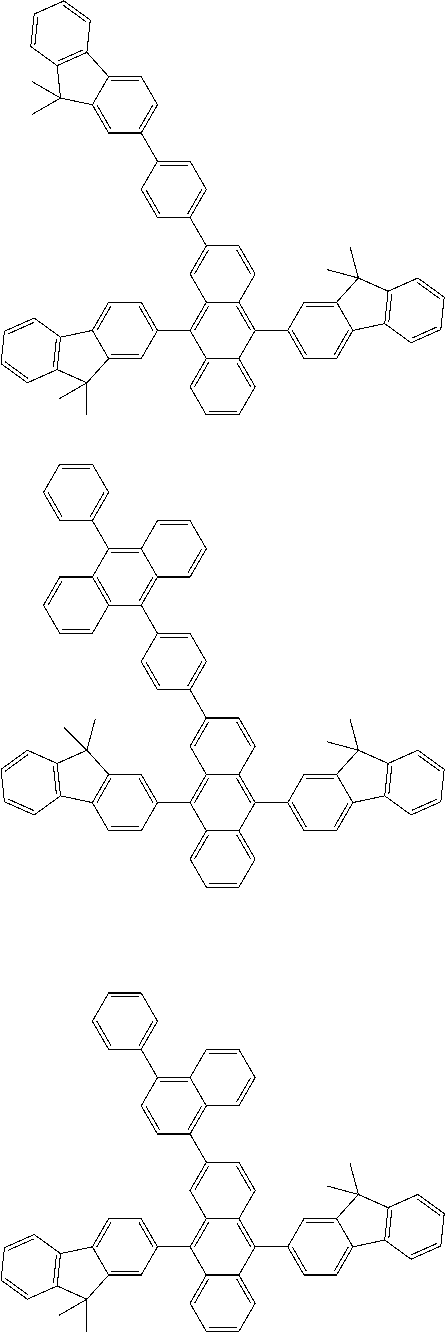

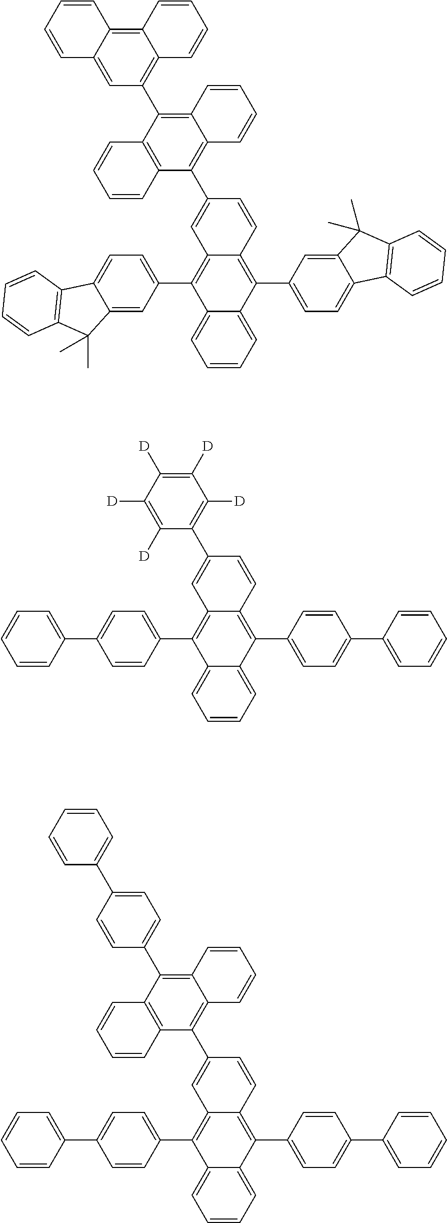

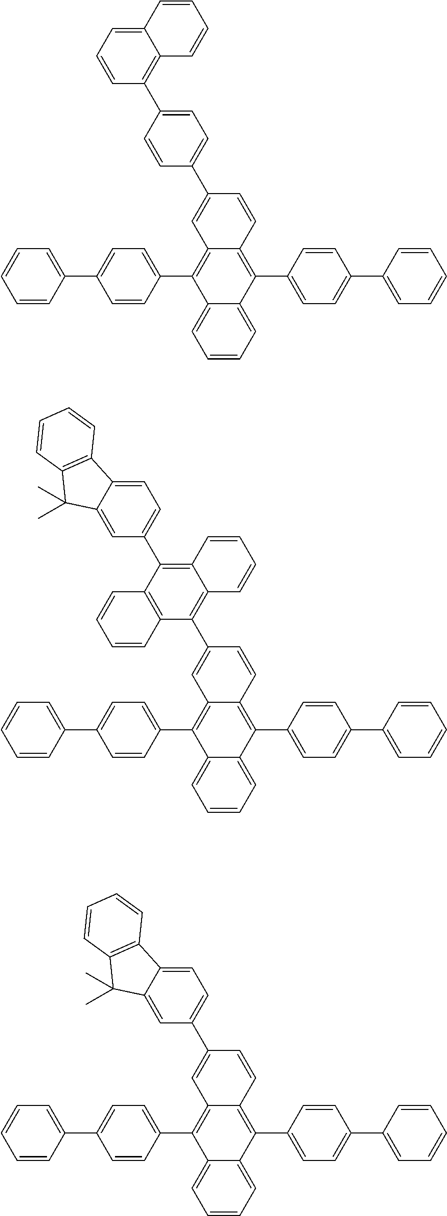

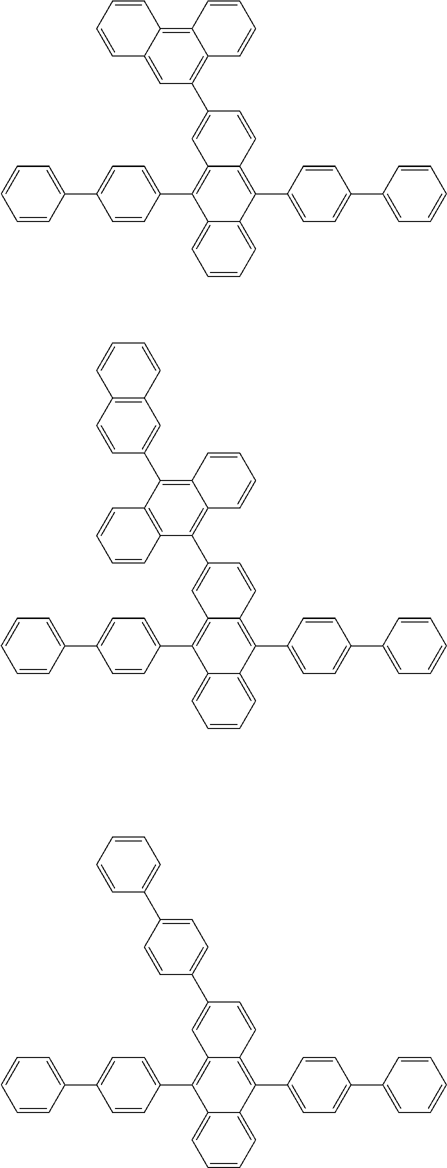

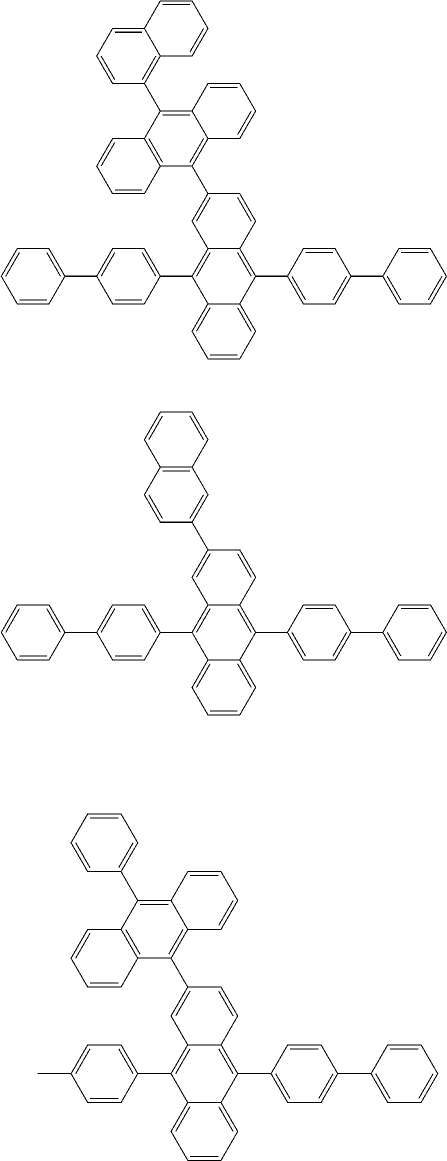

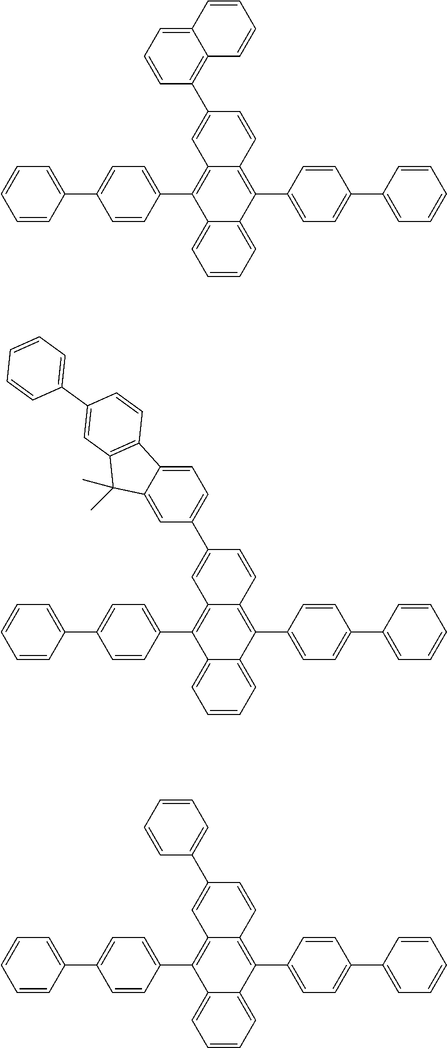

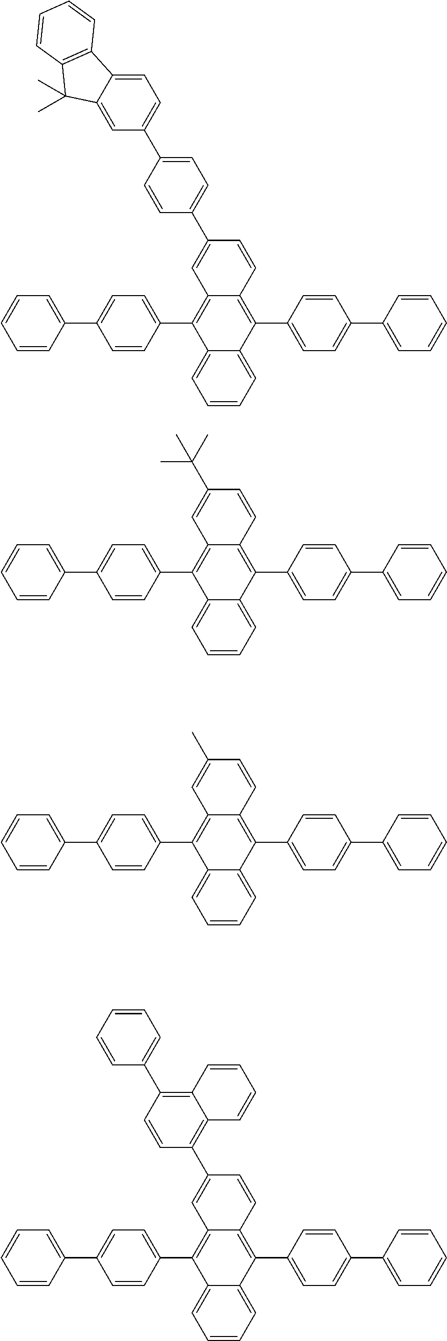

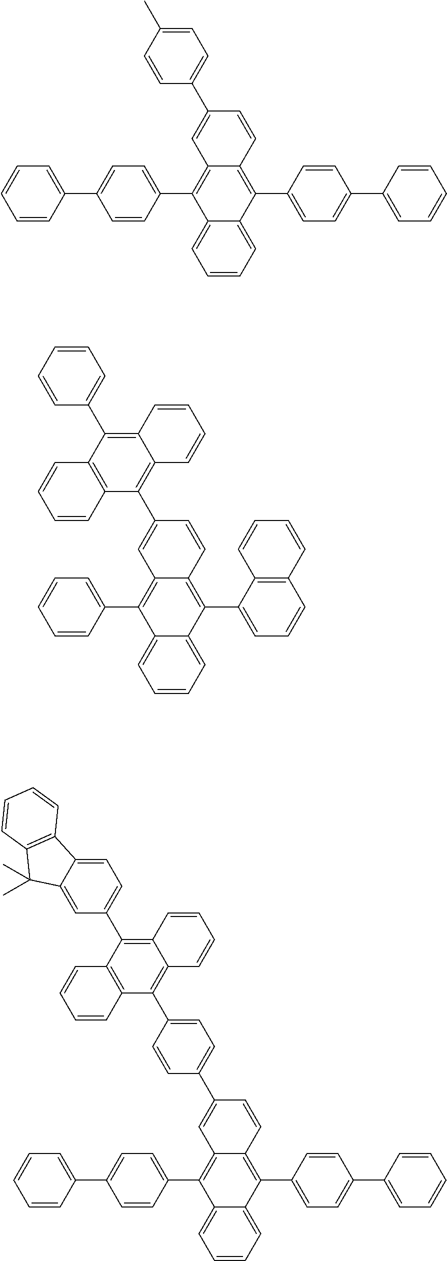

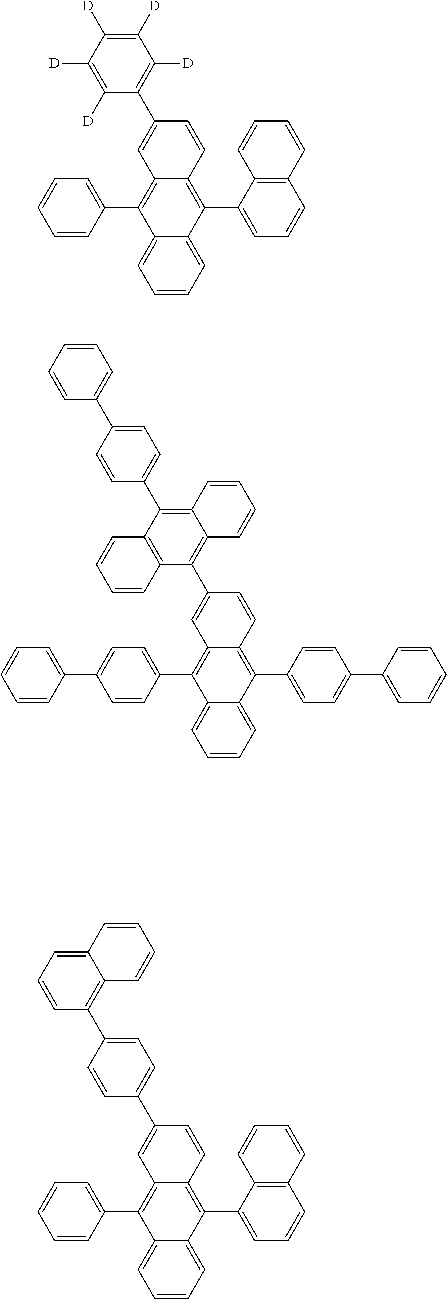

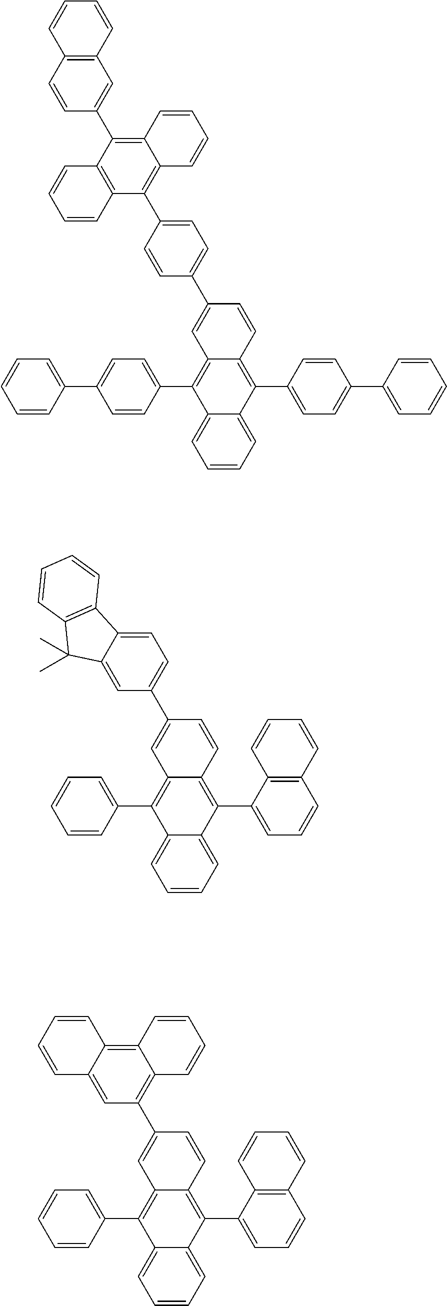

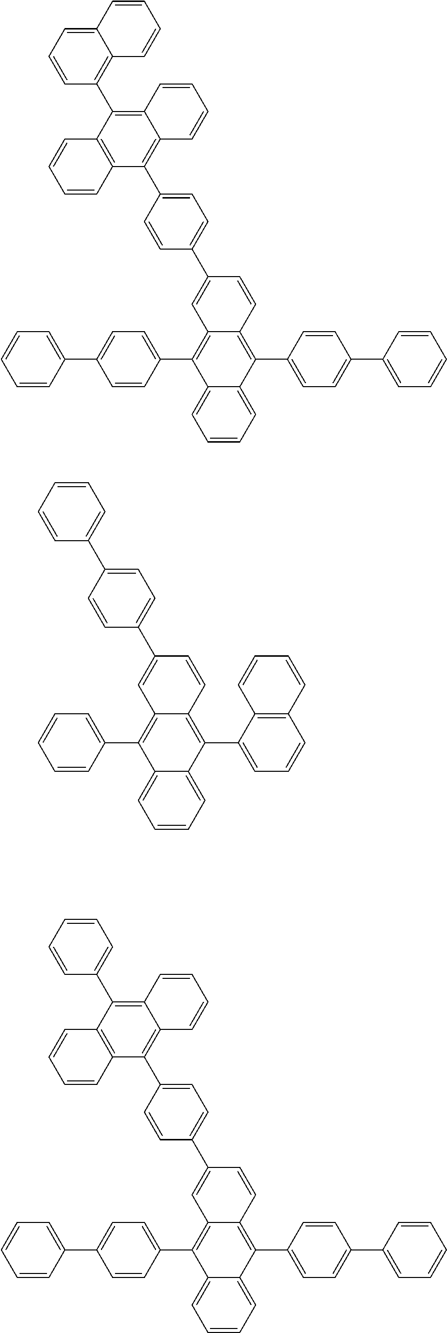

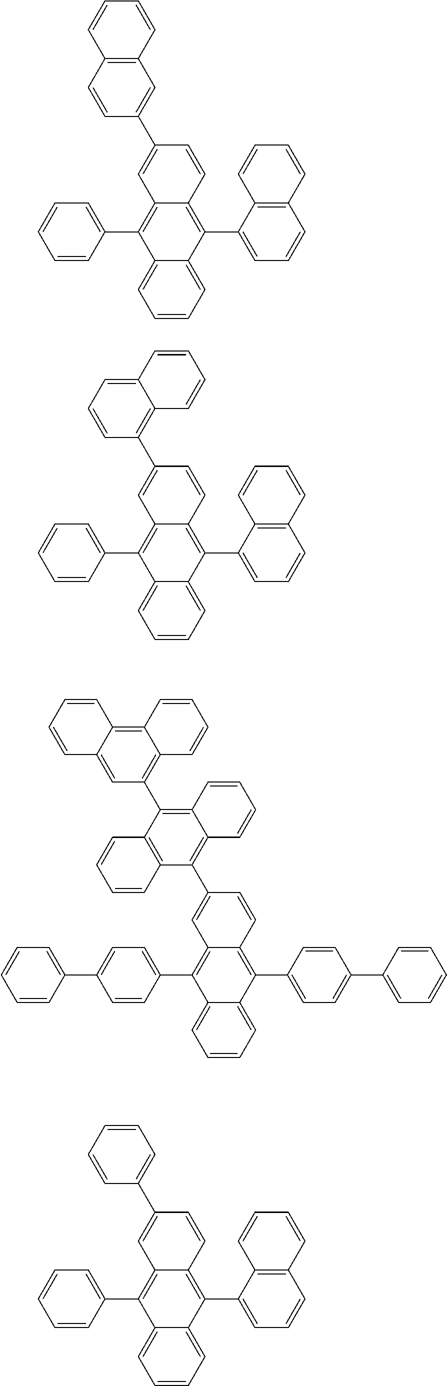

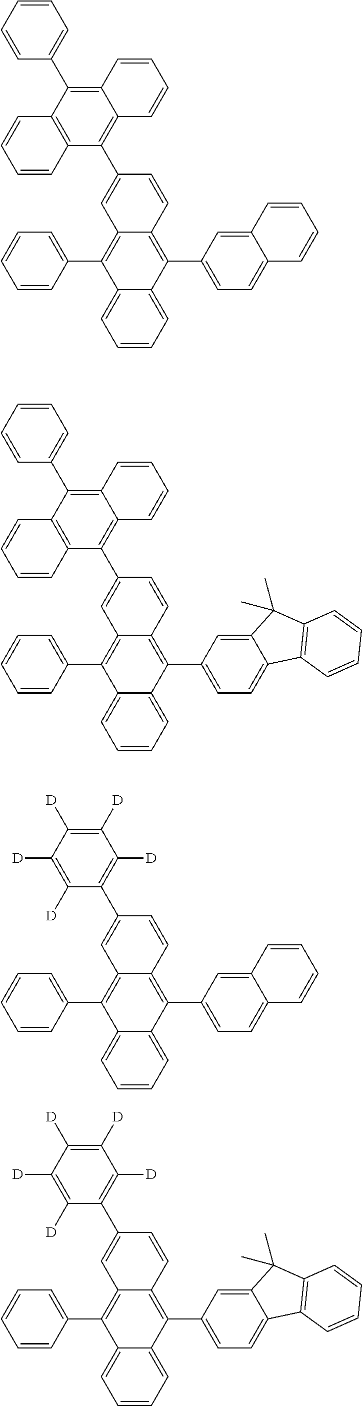

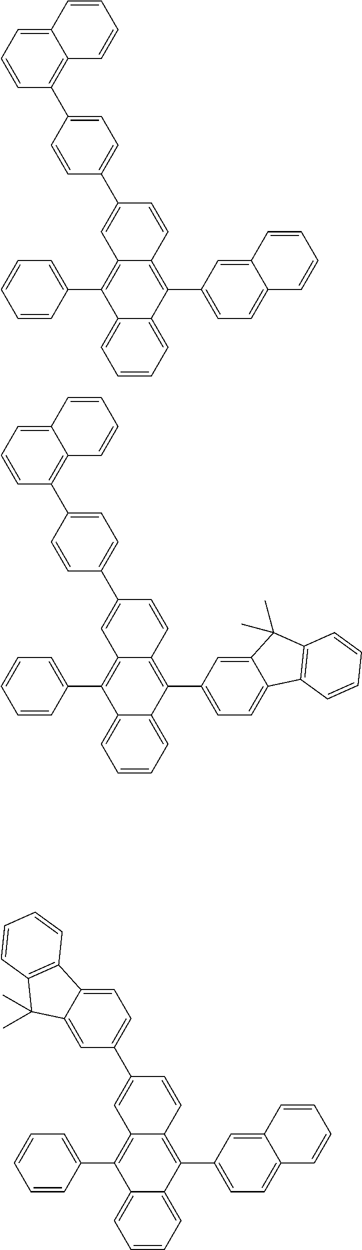

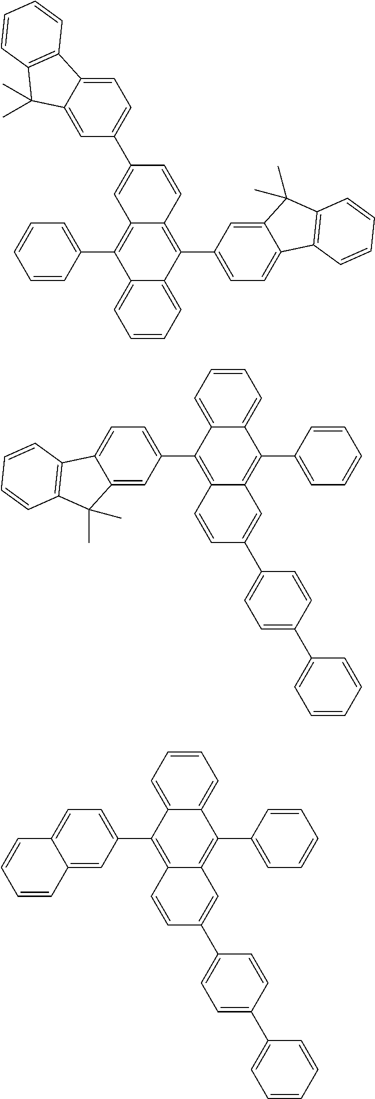

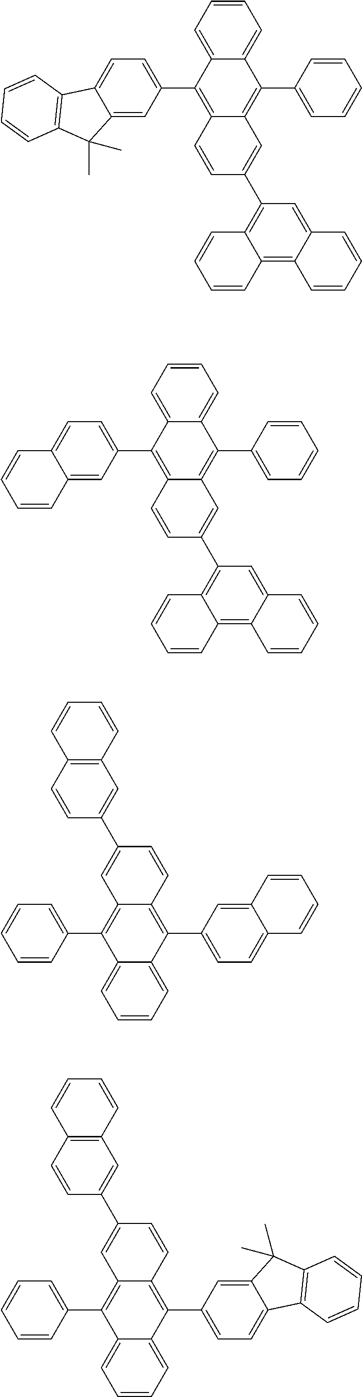

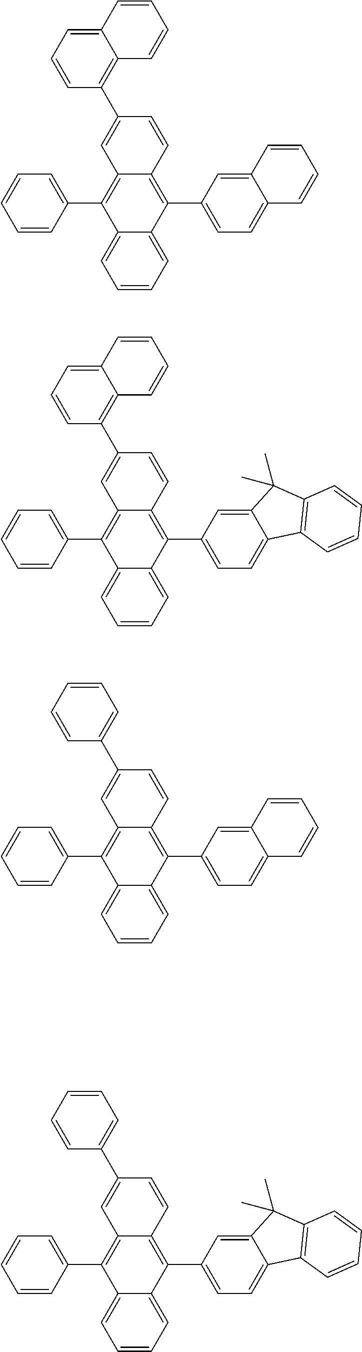

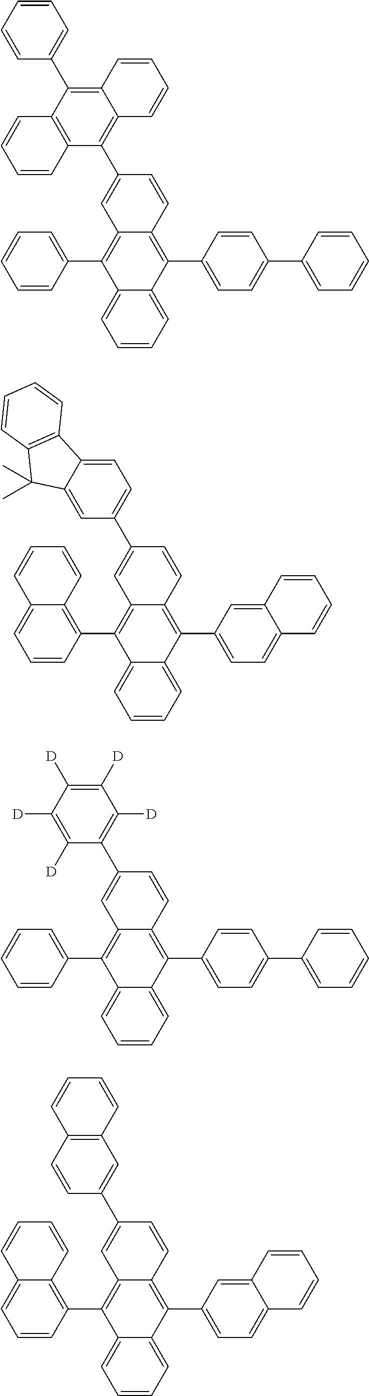

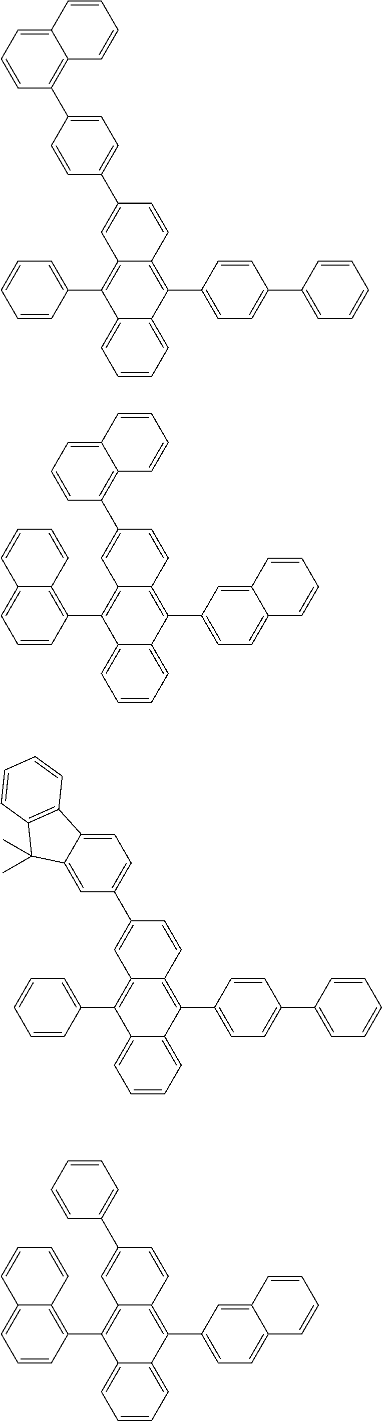

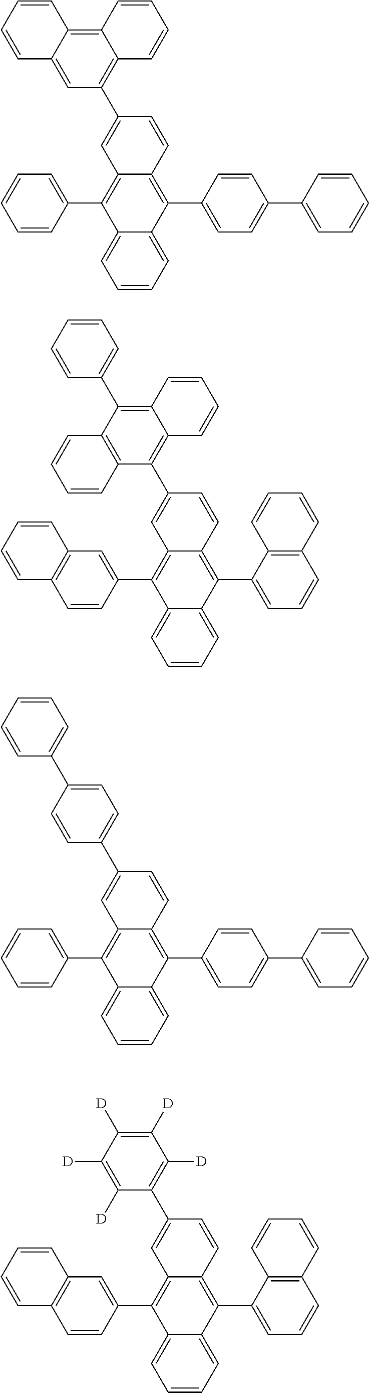

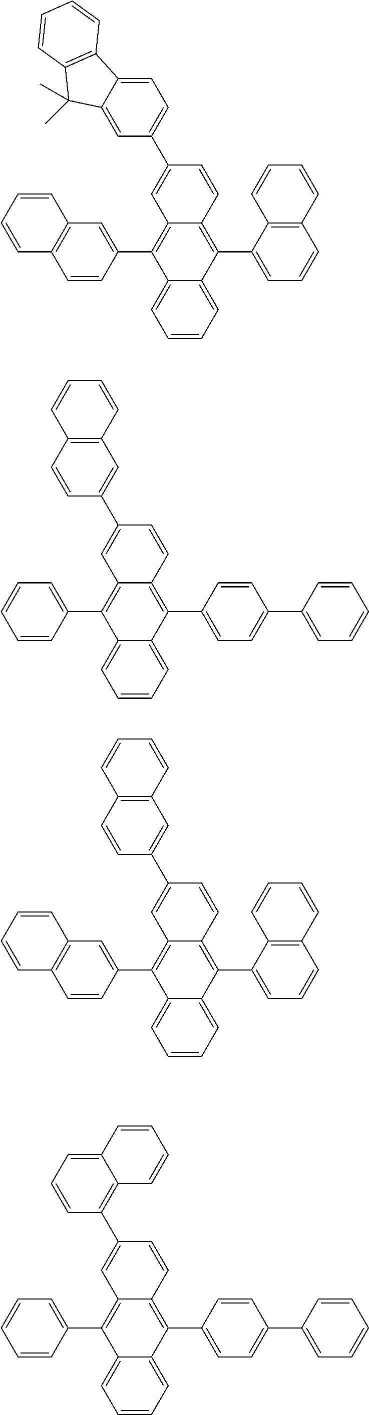

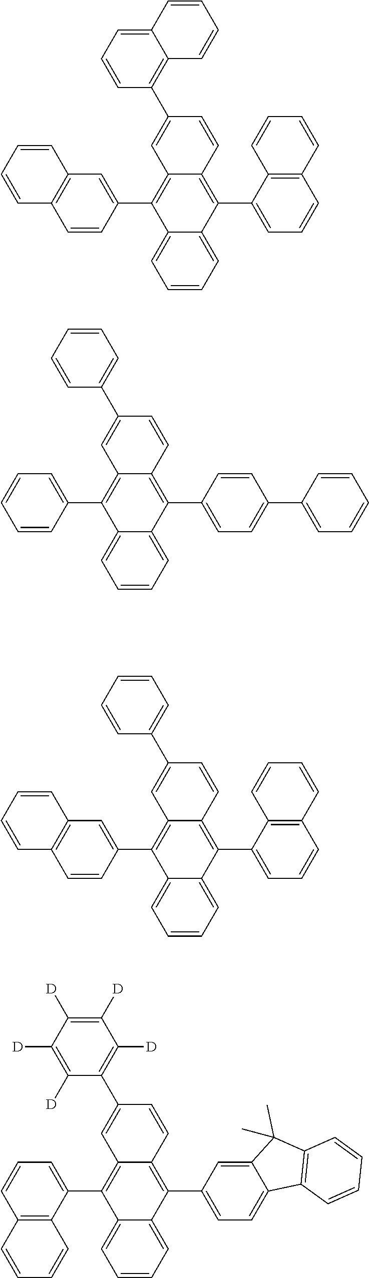

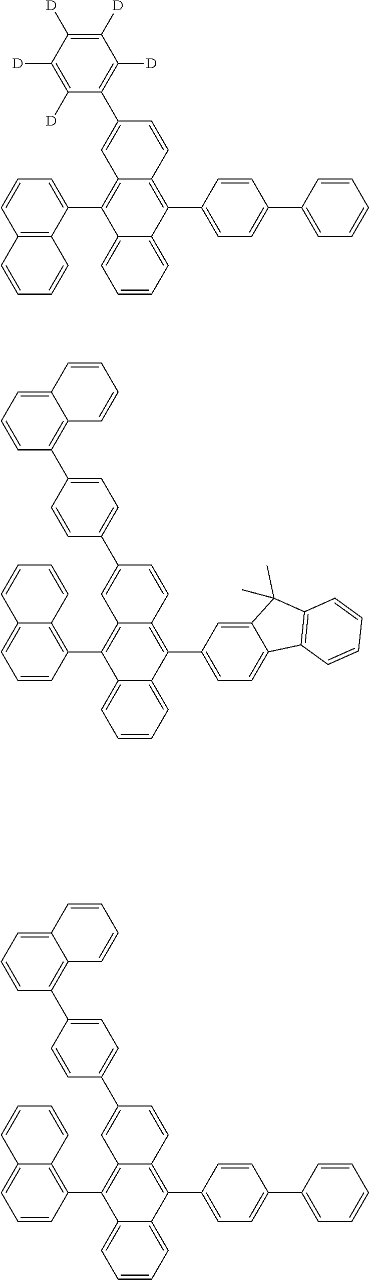

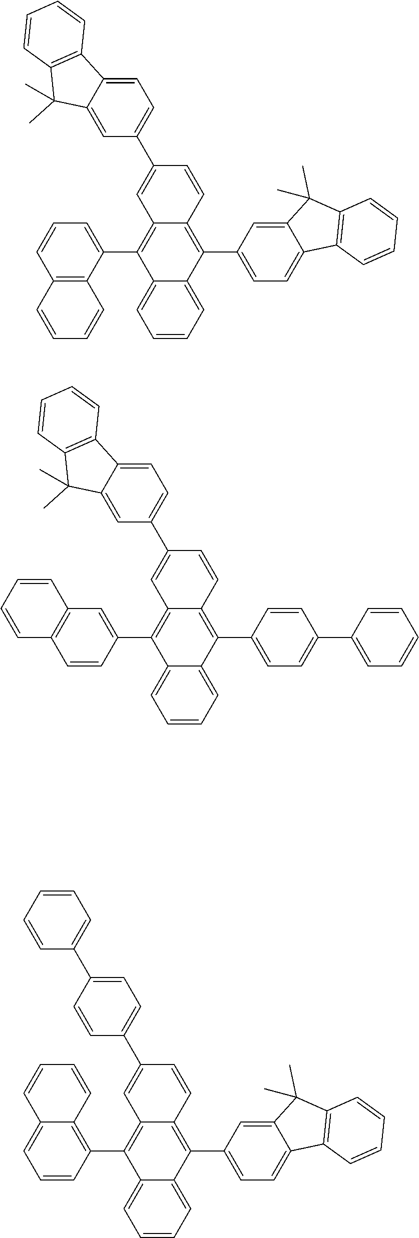

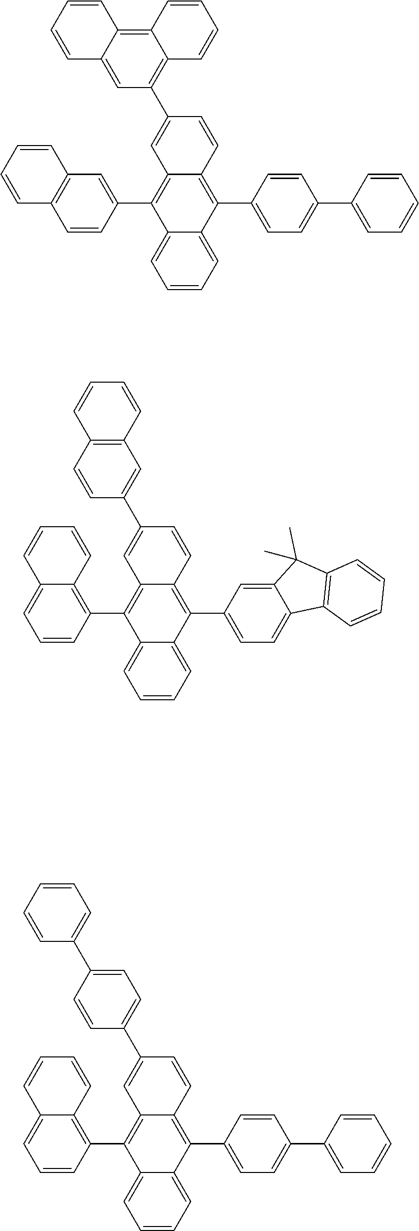

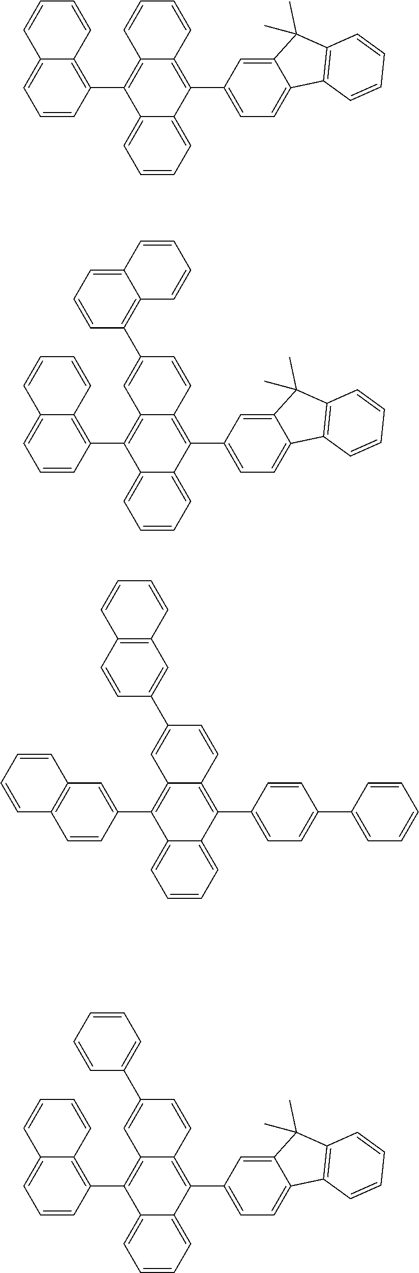

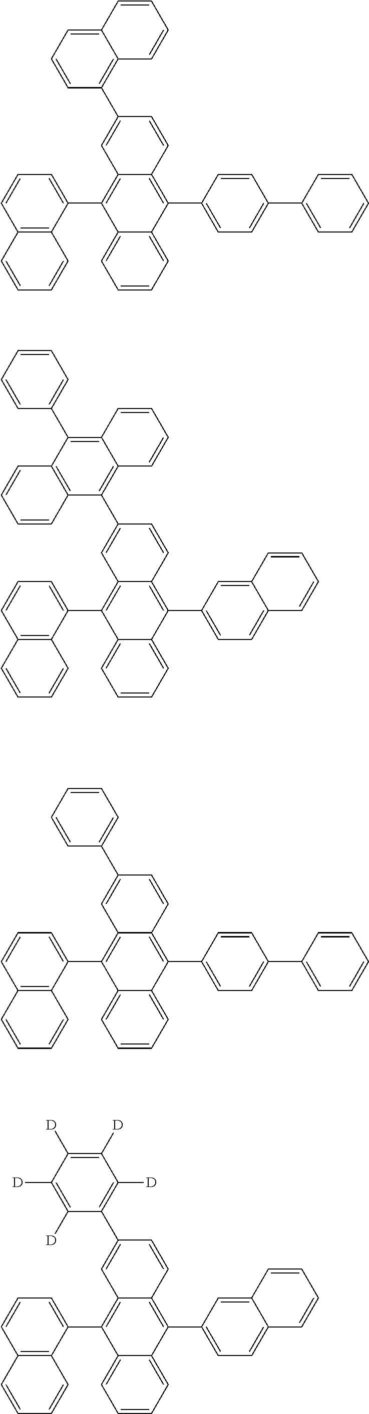

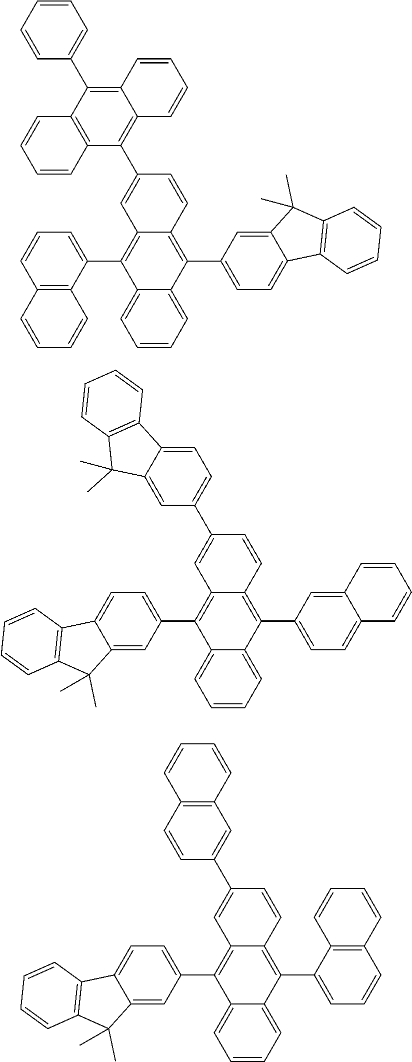

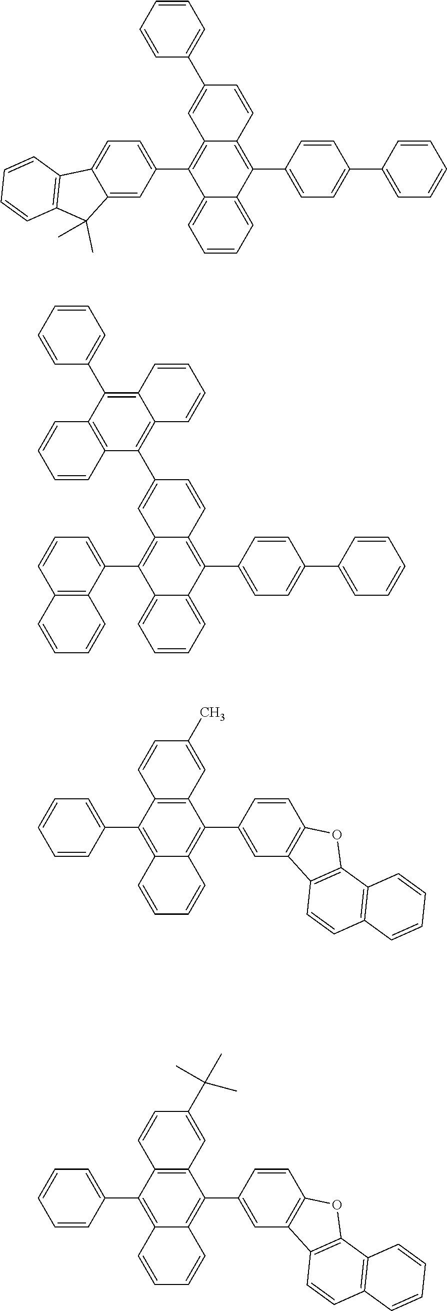

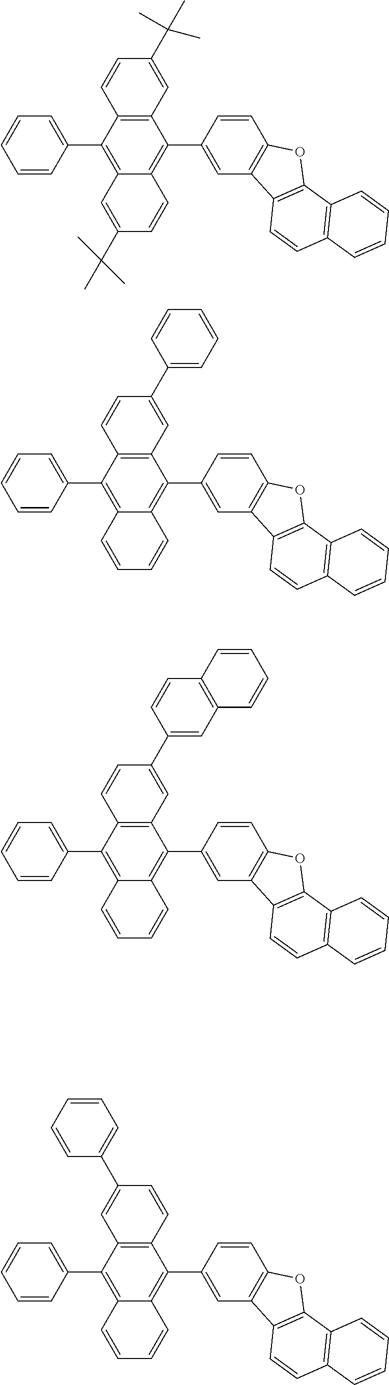 [SMILES: CC(C)(C)c1ccc2c(-c3ccc4oc5c6ccccc6ccc5c4c3)c3cc(C(C)(C)C)ccc3c(-c3ccccc3)c2c1 ; c1ccc(-c2ccc3c(-c4ccccc4)c4ccccc4c(-c4ccc5oc6c7ccccc7ccc6c5c4)c3c2)cc1 ; c1ccc(-c2c3ccccc3c(-c3ccc4oc5c6ccccc6ccc5c4c3)c3cc(-c4ccc5ccccc5c4)ccc23)cc1 ; c1ccc(-c2ccc3c(-c4ccc5oc6c7ccccc7ccc6c5c4)c4ccccc4c(-c4ccccc4)c3c2)cc1]

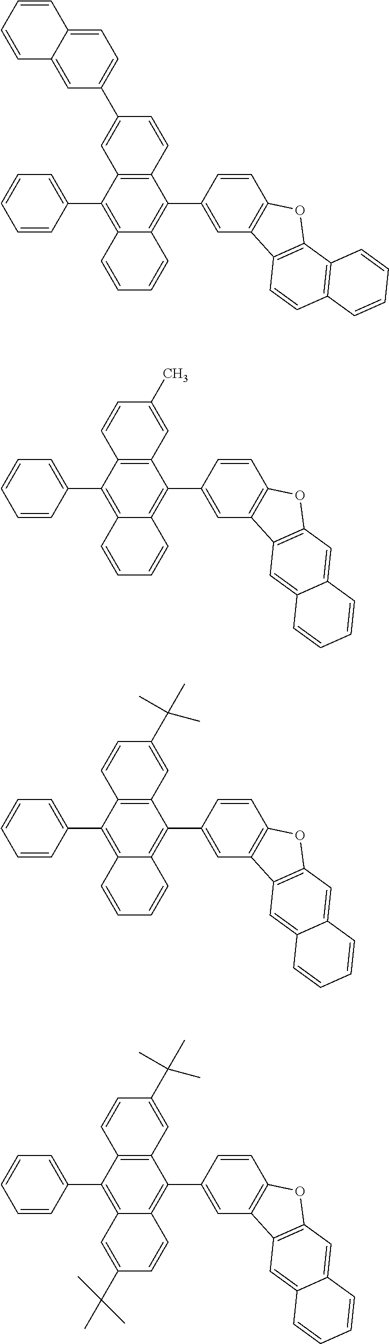 [SMILES: c1ccc(-c2c3ccccc3c(-c3ccc4oc5c6ccccc6ccc5c4c3)c3ccc(-c4ccc5ccccc5c4)cc23)cc1 ; Cc1ccc2c(-c3ccccc3)c3ccccc3c(-c3ccc4oc5cc6ccccc6cc5c4c3)c2c1 ; CC(C)(C)c1ccc2c(-c3ccccc3)c3ccccc3c(-c3ccc4oc5cc6ccccc6cc5c4c3)c2c1 ; CC(C)(C)c1ccc2c(-c3ccc4oc5cc6ccccc6cc5c4c3)c3cc(C(C)(C)C)ccc3c(-c3ccccc3)c2c1]

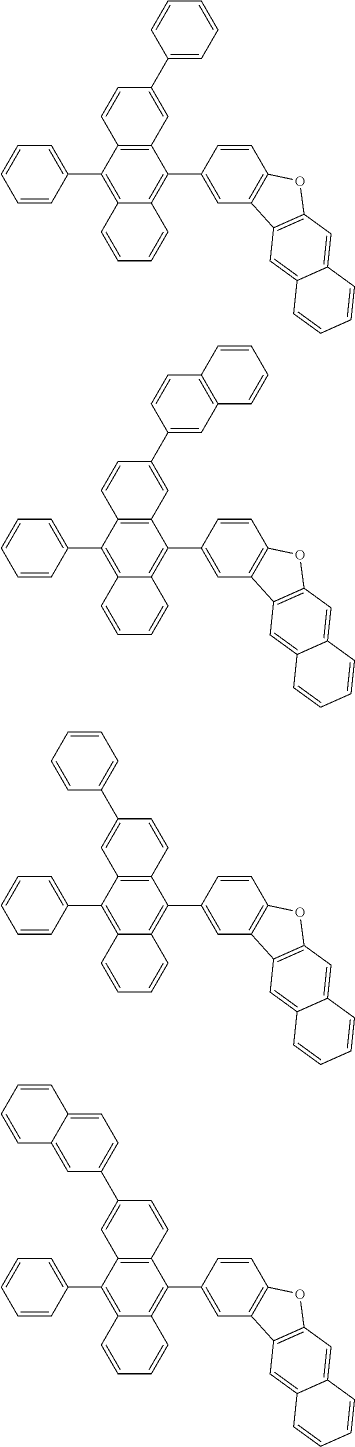 [SMILES: c1ccc(-c2ccc3c(-c4ccccc4)c4ccccc4c(-c4ccc5oc6cc7ccccc7cc6c5c4)c3c2)cc1 ; c1ccc(-c2c3ccccc3c(-c3ccc4oc5cc6ccccc6cc5c4c3)c3cc(-c4ccc5ccccc5c4)ccc23)cc1 ; c1ccc(-c2ccc3c(-c4ccc5oc6cc7ccccc7cc6c5c4)c4ccccc4c(-c4ccccc4)c3c2)cc1 ; c1ccc(-c2c3ccccc3c(-c3ccc4oc5cc6ccccc6cc5c4c3)c3ccc(-c4ccc5ccccc5c4)cc23)cc1]

In the case that the emitting layer comprises the compound represented by formula (I) as a dopant and at least one host, wherein preferred hosts are mentioned above, and the host is more preferably at least one compound represented by formula (10), the content of the at least one compound represented by formula (I) is preferably 1 mass % to 20 mass % relative to the entire mass of the emitting layer.

The content of the at least one host, wherein preferred hosts are mentioned above, preferably the at least one compound represented by formula (10) is preferably 80 mass % to 99 mass % relative to the entire mass of the emitting layer.

An explanation will be made on the layer configuration of the organic EL device according to one aspect of the invention.

An organic EL device according to one aspect of the invention comprises a cathode, an anode, and one or more organic thin film layers comprising an emitting layer disposed between the cathode and the anode. The organic layer comprises at least one layer composed of an organic compound. Alternatively, the organic layer is formed by laminating a plurality of layers composed of an organic compound. The organic layer may further comprise an inorganic compound in addition to the organic compound.

At least one of the organic layers is an emitting layer. The organic layer may be constituted, for example, as a single emitting layer, or may comprise other layers which can be adopted in the layer structure of the organic EL device. The layer that can be adopted in the layer structure of the organic EL device is not particularly limited, but examples thereof include a hole-transporting zone (a hole-transporting layer, a hole-injecting layer, an electron-blocking layer, an exciton-blocking layer, etc.), an emitting layer, a spacing layer, and an electron-transporting zone (electron-transporting layer, electron-injecting layer, hole-blocking layer, etc.) provided between the cathode and the emitting layer.

The organic EL device according to one aspect of the invention may be, for example, a fluorescent or phosphorescent monochromatic light emitting device or a fluorescent/phosphorescent hybrid white light emitting device. Preferably, the organic EL device is a fluorescent monochromatic light emitting device, more preferably a blue fluorescent monochromatic light emitting device or a fluorescent/phosphorescent hybrid white light emitting device. Blue fluorescence means a fluorescence at 400 to 500 nm (peak maximum), preferably at 430 nm to 490 nm (peak maximum).

Further, it may be a simple type device having a single emitting unit or a tandem type device having a plurality of emitting units.

The “emitting unit” in the specification is the smallest unit that comprises organic layers, in which at least one of the organic layers is an emitting layer and light is emitted by recombination of injected holes and electrons.

In addition, the “emitting layer” described in the present specification is an organic layer having an emitting function. The emitting layer is, for example, a phosphorescent emitting layer, a fluorescent emitting layer or the like, preferably a fluorescent emitting layer, more preferably a blue fluorescent emitting layer, and may be a single layer or a stack of a plurality of layers.

The emitting unit may be a stacked type unit having a plurality of phosphorescent emitting layers or fluorescent emitting layers. In this case, for example, a spacing layer for preventing excitons generated in the phosphorescent emitting layer from diffusing into the fluorescent emitting layer may be provided between the respective light-emitting layers.

As the simple type organic EL device, a device configuration such as anode/emitting unit/cathode can be given.

Examples for representative layer structures of the emitting unit are shown below. The layers in parentheses are provided arbitrarily.

(a) (Hole-injecting layer/) Hole-transporting layer/Fluorescent emitting layer (/Electron-transporting layer/Electron-injecting layer)

(b) (Hole-injecting layer/) Hole-transporting layer/Phosphorescent emitting layer (/Electron-transporting layer/Electron-injecting layer)

(c) (Hole-injecting layer/) Hole-transporting layer/First fluorescent emitting layer/Second fluorescent emitting layer (/Electron-transporting layer/Electron-injecting layer)

(d) (Hole-injecting layer/) Hole-transporting layer/First phosphorescent layer/Second phosphorescent layer (/Electron-transporting layer/Electron-injecting layer)

(e) (Hole-injecting layer/) Hole-transporting layer/Phosphorescent emitting layer/Spacing layer/Fluorescent emitting layer (/Electron-transporting layer/Electron-injecting layer)

(f) (Hole-injecting layer/) Hole-transporting layer/First phosphorescent emitting layer/Second phosphorescent emitting layer/Spacing layer/Fluorescent emitting layer (/Electron-transporting layer/Electron-injecting layer)

(g) (Hole-injecting layer/) Hole-transporting layer/First phosphorescent layer/Spacing layer/Second phosphorescent emitting layer/Spacing layer/Fluorescent emitting layer (/Electron-transporting layer/Electron-injecting layer)

(h) (Hole-injecting layer/) Hole-transporting layer/Phosphorescent emitting layer/Spacing layer/First fluorescent emitting layer/Second fluorescent emitting layer (/Electron-transporting Layer/Electron-injecting Layer)

(i) (Hole-injecting layer/) Hole-transporting layer/Electron-blocking layer/Fluorescent emitting layer (/Electron-transporting layer/Electron-injecting layer)

(j) (Hole-injecting layer/) Hole-transporting layer/Electron-blocking layer/Phosphorescent emitting layer (/Electron-transporting layer/Electron-injecting layer)

(k) (Hole-injecting layer/) Hole-transporting layer/Exciton-blocking layer/Fluorescent emitting layer (/Electron-transporting layer/Electron-injecting layer)

(l) (Hole-injecting layer/) Hole-transporting layer/Exciton-blocking layer/Phosphorescent emitting layer (/Electron-transporting layer/Electron-injecting layer)

(m) (Hole-injecting layer/) First hole-transporting Layer/Second hole-transporting Layer/Fluorescent emitting layer (/Electron-transporting layer/electron-injecting Layer)

(n) (Hole-injecting layer/) First hole-transporting layer/Second hole-transporting layer/Fluorescent emitting layer (/First electron-transporting layer/Second electron-transporting layer/Electron-injection layer)

(o) (Hole-injecting layer/) First hole-transporting layer/Second hole-transporting layer/Phosphorescent emitting layer (/Electron-transporting layer/Electron-injecting Layer)

(p) (Hole-injecting layer/) First hole-transporting layer/Second hole-transporting layer/Phosphorescent emitting layer (/First electron-transporting Layer/Second electron-transporting layer/Electron-injecting layer)

(q) (Hole-injecting layer/) Hole-transporting layer/Fluorescent emitting layer/Hole-blocking layer (/Electron-transporting layer/Electron-injecting layer)

(r) (Hole-injecting layer/) Hole-transporting layer/Phosphorescent emitting layer/Hole-blocking layer (/Electron-transport layer/Electron-injecting layer)

(s) (Hole-injecting layer/) Hole-transporting layer/Fluorescent emitting layer/Exciton-blocking layer (/Electron-transporting layer/Electron-injecting layer)

(t) (Hole-injecting layer/) Hole-transporting layer/Phosphorescent emitting layer/Exciton-blocking layer (/Electron-transporting layer/Electron-injecting layer)

The layer structure of the organic EL device according to one aspect of the invention is not limited to the examples mentioned above.

For example, when the organic EL device has a hole-injecting layer and a hole-transporting layer, it is preferred that a hole-injecting layer be provided between the hole-transporting layer and the anode. Further, when the organic EL device has an electron-injecting layer and an electron-transporting layer, it is preferred that an electron-injecting layer be provided between the electron-transporting layer and the cathode. Further, each of the hole-injecting layer, the hole-transporting layer, the electron-transporting layer and the electron-injecting layer may be formed of a single layer or be formed of a plurality of layers.

The plurality of phosphorescent emitting layer, and the plurality of the phosphorescent emitting layer and the fluorescent emitting layer may be emitting layers that emit mutually different colors. For example, the emitting unit (f) may include a hole-transporting layer/first phosphorescent layer (red light emission)/second phosphorescent emitting layer (green light emission)/spacing layer/fluorescent emitting layer (blue light emission)/electron-transporting layer.

An electron-blocking layer may be provided between each light emitting layer and the hole-transporting layer or the spacing layer. Further, a hole-blocking layer may be provided between each emitting layer and the electron-transporting layer. By providing the electron-blocking layer or the hole-blocking layer, it is possible to confine electrons or holes in the emitting layer, thereby to improve the recombination probability of carriers in the emitting layer, and to improve light emitting efficiency.

As a representative device configuration of a tandem type organic EL device, for example, a device configuration such as anode/first emitting unit/intermediate layer/second emitting unit/cathode can be given.

The first emitting unit and the second emitting unit are independently selected from the above-mentioned emitting units, for example.

The intermediate layer is also generally referred to as an intermediate electrode, an intermediate conductive layer, a charge generating layer, an electron withdrawing layer, a connecting layer, a connector layer, or an intermediate insulating layer. The intermediate layer is a layer that supplies electrons to the first emitting unit and holes to the second emitting unit, and can be formed from known materials.

FIG. 1 shows a schematic configuration of one example of the organic EL device of the invention. The organic EL device 1 comprises a substrate 2, an anode 3, a cathode 4 and an emitting unit 10 provided between the anode 3 and the cathode 4. The emitting unit 10 comprises an emitting layer 5 preferably comprising a host material and a dopant. A hole injecting and transporting layer 6 or the like may be provided between the emitting layer 5 and the anode 3 and an electron injecting layer 8 and an electron transporting layer 7 or the like (electron injecting and transporting unit 11) may be provided between the emitting layer 5 and the cathode 4. An electron-barrier layer may be provided on the anode 3 side of the emitting layer 5 and a hole-barrier layer may be provided on the cathode 4 side of the emitting layer 5. Due to such configuration, electrons or holes can be confined in the emitting layer 5, whereby possibility of generation of excitons in the emitting layer 5 can be improved.

Hereinbelow, an explanation will be made on function, materials, etc. of each layer constituting the organic EL device described in the present specification.

(Substrate)

The substrate is used as a support of the organic EL device. The substrate preferably has a light transmittance of 50% or more in the visible light region with a wavelength of 400 to 700 nm, and a smooth substrate is preferable. Examples of the material of the substrate include soda-lime glass, aluminosilicate glass, quartz glass, plastic and the like. As a substrate, a flexible substrate can be used. The flexible substrate means a substrate that can be bent (flexible), and examples thereof include a plastic substrate and the like. Specific examples of the material for forming the plastic substrate include polycarbonate, polyallylate, polyether sulfone, polypropylene, polyester, polyvinyl fluoride, polyvinyl chloride, polyimide, polyethylene naphthalate and the like. Also, an inorganic vapor deposited film can be used.

(Anode)

As the anode, for example, it is preferable to use a metal, an alloy, a conductive compound, a mixture thereof or the like and having a high work function (specifically, 4.0 eV or more). Specific examples of the material of the anode include indium oxide-tin oxide (ITO: Indium Tin Oxide), indium oxide-tin oxide containing silicon or silicon oxide, indium oxide-zinc oxide, indium oxide containing tungsten oxide or zinc oxide, graphene and the like. In addition, it is also possible to use gold, silver, platinum, nickel, tungsten, chromium, molybdenum, iron, cobalt, copper, palladium, titanium, and nitrides of these metals (e.g. titanium oxide).

The anode is normally formed by depositing these materials on the substrate by a sputtering method. For example, indium oxide-zinc oxide can be formed by a sputtering method by using a target in which 1 to 10 mass % zinc oxide is added relative to indium oxide. Further, indium oxide containing tungsten oxide or zinc oxide can be formed by a sputtering method by using a target in which 0.5 to 5 mass % of tungsten oxide or 0.1 to 1 mass % of zinc oxide is added relative to indium oxide.

As other methods for forming the anode, a vacuum deposition method, a coating method, an inkjet method, a spin coating method or the like can be given. When silver paste or the like is used, it is possible to use a coating method, an inkjet method or the like.

The hole-injecting layer formed in contact with the anode is formed by using a material that allows easy hole injection regardless of the work function of the anode. For this reason, in the anode, it is possible to use a common electrode material, e.g. a metal, an alloy, a conductive compound and a mixture thereof. Specifically, a material having a small work function such as alkaline metals such as lithium and cesium; alkaline earth metals such as calcium and strontium; alloys containing these metals (for example, magnesium-silver and aluminum-lithium); rare earth metals such as europium and ytterbium; and an alloy containing rare earth metals.

(Hole-Transporting Layer)/(Hole-Injecting Layer)

The hole-transporting layer is an organic layer that is formed between the emitting layer and the anode, and has a function of transporting holes from the anode to the emitting layer. If the hole-transporting layer is composed of plural layers, an organic layer that is nearer to the anode may often be defined as the hole-injecting layer. The hole-injecting layer has a function of injecting holes efficiently to the organic layer unit from the anode. Said hole injection layer is generally used for stabilizing hole injection from anode to hole transporting layer which is generally consist of organic materials. Organic material having good contact with anode or organic material with p-type doping is preferably used for the hole injection layer.

p-doping usually consists of one or more p-dopant materials and one or more matrix materials. Matrix materials preferably have shallower HOMO level and p-dopant preferably have deeper LUMO level to enhance the carrier density of the layer. Aryl or heteroaryl amine compounds are preferably used as the matrix materials. Specific examples for the matrix material are the same as that for hole transporting layer which is explained at the later part. Specific examples for p-dopant are the below mentioned acceptor materials, preferably the quinone compounds with one or more electron withdrawing groups, such as F₄TCNQ, 1,2,3-Tris[(cyano)(4-cyano-2,3,5,6-tetrafluorophenyl)methylene]cyclopropane.

Acceptor materials, or fused aromatic hydrocarbon materials or fused heterocycles which have high planarity, are preferably used as p-dopant materials for the hole injection layer. Specific examples for acceptor materials are, the quinone compounds with one or more electron withdrawing groups, such as F₄TCNQ(2,3,5,6-tetrafluoro-7,7,8,8-tetracyanoquinodimethane), and 1,2,3-tris[(cyano)(4-cyano-2,3,5,6-tetrafluorophenyl)methylene]cyclopropane; hexa-azatriphenylene compounds with one or more electron withdrawing groups, such as hexa-azatriphenylene-hexanitrile; aromatic hydrocarbon compounds with one or more electron withdrawing groups; and aryl boron compounds with one or more electron withdrawing groups.

The ratio of the p-type dopant is preferably less than 20% of molar ratio, more preferably less than 10%, such as 1%, 3%, or 5%, related to the matrix material.

The hole transporting layer is generally used for injecting and transporting holes efficiently, and aromatic or heterocyclic amine compounds are preferably used.

Specific examples for compounds for the hole transporting layer are represented by the general formula (H),

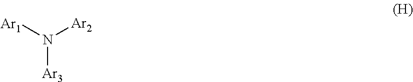

wherein

Ar₁ to Ar₃ each independently represents substituted or unsubstituted aryl group having 5 to 50 carbon atoms or substituted or unsubstituted heterocyclic group having 5 to 50 cyclic atoms, preferably phenyl group, biphenyl group, terphenyl group, naphthyl group, phenanthryl group, triphenylenyl group, fluorenyl group, spirobifluorenyl group, indenofluorenyl group, carbazolyl group, dibenzofuranyl group, dibenzothiophenyl group, carbazole substituted aryl group, dibenzofuran substituted aryl group or dibenzothiophene substituted aryl group; two or more substituents selected among Ar¹ to Ar³ may be bonded to each other to form a ring structure, such as a carbazole ring structure, or a acridane ring structure.

Preferably, at least one of Ar₁ to Ar₃ have additional one aryl or heterocyclic amine substituent, more preferably Ar₁ has an additional aryl amino substituent, at the case of that it is preferable that Ar₁ represents substituted or unsubstituted biphenylene group, substituted or unsubstituted fluorenylene group.

A second hole transporting layer is preferably inserted between the first hole transporting layer and the emitting layer to enhance device performance by blocking excess electrons or excitons.

Specific examples for second hole transporting layer are the same as for the the first hole transporting layer. It is preferred that second hole transporting layer has higher triplet energy to block triplet excitons, especially for phosphorescent green device, such as bicarbazole compounds, biphenylamine compounds, triphenylenyl amine compounds, fluorenyl amine compounds, carbazole substituted arylamine compounds, dibenzofuran substituted arylamine compounds, and dibenzothiophene substituted arylamine compounds.

(Emitting layer)

The emitting layer is a layer containing a substance having a high emitting property (emitter material or dopant material). As the dopant material, various materials can be used. For example, a fluorescent emitting compound (fluorescent dopant), a phosphorescent emitting compound (phosphorescent dopant) or the like can be used. A fluorescent emitting compound is a compound capable of emitting light from the singlet excited state, and an emitting layer containing a fluorescent emitting compound is called a fluorescent emitting layer. Further, a phosphorescent emitting compound is a compound capable of emitting light from the triplet excited state, and an emitting layer containing a phosphorescent emitting compound is called a phosphorescent emitting layer.

Preferably, the emitting layer in the organic EL device of the present application comprises a compound of formula (I) as a dopant material.

The emitting layer preferably comprises at least one dopant material and at least one host material that allows it to emit light efficiently. In some literatures, a dopant material is called a guest material, an emitter or an emitting material. In some literatures, a host material is called a matrix material.

A single emitting layer may comprise plural dopant materials and plural host materials. Further, plural emitting layers may be present.

In the present specification, a host material combined with the fluorescent dopant is referred to as a “fluorescent host” and a host material combined with the phosphorescent dopant is referred to as the “phosphorescent host”. Note that the fluorescent host and the phosphorescent host are not classified only by the molecular structure. The phosphorescent host is a material for forming a phosphorescent emitting layer containing a phosphorescent dopant, but does not mean that it cannot be used as a material for forming a fluorescent emitting layer. The same can be applied to the fluorescent host.

In one embodiment, it is preferred that the emitting layer comprise the compound represented by formula (I) according to the present invention (hereinafter, these compounds may be referred to as the “compound (I)”). More preferably, it is contained as a dopant material. Further, it is preferred that the compound (I) be contained in the emitting layer as a fluorescent dopant. Even further, it is preferred that the compound (I) be contained in the emitting layer as a blue fluorescent dopant.

In one embodiment, no specific restrictions are imposed on the content of the compound (I) as the dopant material in the emitting layer. In respect of sufficient emission and concentration quenching, the content is preferably 0.5 to 70 mass %, more preferably 0.8 to 30 mass %, further preferably 1 to 30 mass %, still further preferably 1 to 20 mass %, and particularly preferably 1 to 10 mass %, further particularly preferably 1 to 5 mass %, even further particularly preferably 2 to 4 mass %, related to the mass of the emitting layer.

(Fluorescent Dopant)

As a fluorescent dopant other than the compound (I), a fused polycyclic aromatic compound, a styrylamine compound, a fused ring amine compound, a boron-containing compound, a pyrrole compound, an indole compound, a carbazole compound can be given, for example. Among these, a fused ring amine compound, a boron-containing compound, carbazole compound is preferable.

As the fused ring amine compound, a diaminopyrene compound, a diaminochrysene compound, a diaminoanthracene compound, a diaminofluorene compound, a diaminofluorene compound with which one or more benzofuro skeletons are fused, or the like can be given.

As the boron-containing compound, a pyrromethene compound, a triphenylborane compound or the like can be given.

As a blue fluorescent dopant, pyrene compounds, styrylamine compounds, chrysene compounds, fluoranthene compounds, fluorene compounds, diamine compounds, triarylamine compounds and the like can be given, for example. Specifically, N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenyamine (abbreviation: YGAPA), 4-(10-phenyl-9-anthryl)-4′-(9-phenyl-9H-carbazole-3-yl)triphenylamine (abbreviation: PCBAPA) or the like can be given.

As a green fluorescent dopant, an aromatic amine compound or the like can be given, for example. Specifically, N-(9,10-diphenyl-2-anthryl)-N,9-diphenyl-9H-carbazole-3-amine (abbreviation: 2PCAPA), N-[9,10-bis(1,1′-biphenyl-2-yl)-2-anthryl]-N,9-diphenyl-9H-carbazole-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), N-[9,10-bis(1,1′-biphenyl-2-yl)]-N-[4-(9H-carbazole-9-yl)phenyl]-N-phenylanthracene-2-amine (abbreviation: 2YGABPhA), N,N,9-triphenylanthracene-9-amine (abbreviation: DPhAPhA) or the like can be given, for example.

As a red fluorescent dopant, a tetracene compound, a diamine compound or the like can be given. Specifically, N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD) or the like can be given.

(Phosphorescent Dopant)

As a phosphorescent dopant, a phosphorescent emitting heavy metal complex and a phosphorescent emitting rare earth metal complex can be given.

As the heavy metal complex, an iridium complex, an osmium complex, a platinum complex or the like can be given. The heavy metal complex is for example an ortho-metalated complex of a metal selected from iridium, osmium and platinum.

Examples of rare earth metal complexes include terbium complexes, europium complexes and the like. Specifically, tris(acetylacetonate)(monophenanthroline)terbium(III) (abbreviation: Tb(acac)₃(Phen)), tris(1,3-diphenyl-1,3-propandionate)(monophenanthroline)europium(III) (abbreviation: Eu(DBM)₃(Phen)), tris[1-(2-thenoyl)-3,3,3-trifluoroacetonate](monophenanthroline)europium(II) (abbreviation: Eu(TTA)₃(Phen)) or the like can be given. These rare earth metal complexes are preferable as phosphorescent dopants since rare earth metal ions emit light due to electronic transition between different multiplicity.

As a blue phosphorescent dopant, an iridium complex, an osmium complex, a platinum complex, or the like can be given, for example. Specifically, bis[2-(4′,6′-difluorophenyl)pyridinate-N,C2′]iridium(III) tetrakis(1-pyrazolyl)borate (abbreviation: Flr6), bis[2-(4′,6′-difluorophenyl) pyridinato-N,C2′]iridium(III) picolinate (abbreviation: Ir(CF₃ppy)₂(pic)), bis[2-(4′,6′-difluorophenyl)pyridinato-N,C2′]iridium(III) acetylacetonate (abbreviation: Flracac) or the like can be given.

As a green phosphorescent dopant, an iridium complex or the like can be given, for example. Specifically, tris(2-phenylpyridinato-N,C2′) iridium(III) (abbreviation: Ir(ppy)₃), bis(1,2-diphenyl-1H-benzimidazolato)iridium(III) acetylacetonate (abbreviation: Ir(pbi)₂(acac)), bis(benzo[h]quinolinato)iridium(II) acetylacetonate (abbreviation: Ir(bzq)₂(acac)) or the like can be given.

As a red phosphorescent dopant, an iridium complex, a platinum complex, a terbium complex, an europium complex or the like can be given. Specifically, bis[2-(2′-benzo[4,5-a]thienyl)pyridinato-N,C3′]iridium(III) acetylacetonate (abbreviation: Ir(btp)₂(acac)), bis(1-phenylisoquinolinato-N,C2′)iridium(III) acetylacetonate (abbreviation: Ir(piq)₂(acac)), (acetylacetonato)bis[2,3-bis(4-fluorophenyl)quinoxalinato]iridium(Ill) (abbreviation: Ir(Fdpq)₂(acac)), 2,3,7,8,12,13,17,18-octaethyl-21H,23H-porphyrin platinum(II) (abbreviation PtOEP) or the like can be given.

As mentioned above, the emitting layer preferably comprises at least one compound (I) as a dopant.

(Host Material)

As host material, metal complexes such as aluminum complexes, beryllium complexes and zinc complexes; heterocyclic compounds such as indole compounds, pyridine compounds, pyrimidine compounds, triazine compounds, quinoline compounds, isoquinoline compounds, quinazoline compounds, dibenzofuran compounds, dibenzothiophene compounds, oxadiazole compounds, benzimidazole compounds, phenanthroline compounds; fused polyaromatic hydrocarbon (PAH) compounds such as a naphthalene compound, a triphenylene compound, a carbazole compound, an anthracene compound, a phenanthrene compound, a pyrene compound, a chrysene compound, a naphthacene compound, a fluoranthene compound; and aromatic amine compound such as triarylamine compounds and fused polycyclic aromatic amine compounds can be given, for example. Plural types of host materials can be used in combination.

As a fluorescent host, a compound having a higher singlet energy level than a fluorescent dopant is preferable. For example, a heterocyclic compound, a fused aromatic compound or the like can be given. As a fused aromatic compound, an anthracene compound, a pyrene compound, a chrysene compound, a naphthacene compound or the like are preferable. An anthracene compound is preferentially used as blue fluorescent host.

As a phosphorescent host, a compound having a higher triplet energy level as compared with a phosphorescent dopant is preferable. For example, a metal complex, a heterocyclic compound, a fused aromatic compound or the like can be given. Among these, an indole compound, a carbazole compound, a pyridine compound, a pyrimidine compound, a triazine compound, a quinolone compound, an isoquinoline compound, a quinazoline compound, a dibenzofuran compound, a dibenzothiophene compound, a naphthalene compound, a triphenylene compound, a phenanthrene compound, a fluoranthene compound or the like can be given.

In the case that compound (I) is employed as at least one dopant material, preferred host materials are substituted or unsubstituted polyaromatic hydrocarbon (PAH) compounds, substituted or unsubstituted polyheteroaromatic compounds, substituted or unsubstituted anthracene compounds, or substituted or unsubstituted pyrene compounds, preferably substituted or unsubstituted anthracene compounds or substituted or unsubstituted pyrene compounds, more preferably substituted or unsubstituted anthracene compounds, most preferably anthracene compounds represented by formula (10), as mentioned above.

(Electron-Transporting Layer)/(Electron-Injecting Layer)

The electron-transporting layer is an organic layer that is formed between the emitting layer and the cathode and has a function of transporting electrons from the cathode to the emitting layer. When the electron-transporting layer is formed of plural layers, an organic layer or an inorganic layer that is nearer to the cathode is often defined as the electron injecting layer (see for example layer 8 in FIG. 1, wherein an electron injecting layer 8 and an electron transporting layer 7 form an electron injecting and transporting unit 11). The electron injecting layer has a function of injecting electrons from the cathode efficiently to the organic layer unit. Preferred electron injection materials are alkali metal, alkali metal compounds, alkali metal complexes, the alkaline earth metal complexes and the rare earth metal complexes. According to one embodiment, it is preferred that the electron-transporting layer further comprises one or more layer(s) like an electron injection layer to enhance efficiency and lifetime of the device, a hole blocking layer, an exciton blocking layer or a triplet blocking layer.

In one embodiment of the present invention, the compound of the formula (I) is present in the electron transporting layer, as an electron transporting material, an electron injecting material, a hole blocking material, a exciton blocking material and/or a triplet blocking material.

According to one embodiment, it is preferred that an electron-donating dopant be contained in the interfacial region between the cathode and the emitting unit. Due to such a configuration, the organic EL device can have an increased luminance or a long life. Here, the electron-donating dopant means one having a metal with a work function of 3.8 eV or less. As specific examples thereof, at least one selected from an alkali metal, an alkali metal complex, an alkali metal compound, an alkaline earth metal, an alkaline earth metal complex, an alkaline earth metal compound, a rare earth metal, a rare earth metal complex and a rare earth metal compound or the like can be mentioned.

As the alkali metal, Li (work function: 2.9 eV), Na (work function: 2.36 eV), K (work function: 2.28 eV), Rb (work function: 2.16 eV), Cs (work function: 1.95 eV) and the like can be given. One having a work function of 2.9 eV or less is particularly preferable. Among them, K, Rb and Cs are preferable. Rb or Cs is further preferable. Cs is most preferable. As the alkaline earth metal, Ca (work function: 2.9 eV), Sr (work function: 2.0 eV to 2.5 eV), Ba (work function: 2.52 eV) and the like can be given. One having a work function of 2.9 eV or less is particularly preferable. As the rare-earth metal, Sc, Y, Ce, Tb, Yb and the like can be given. One having a work function of 2.9 eV or less is particularly preferable.

Examples of the alkali metal compound include an alkali oxide such as Li₂O, Cs₂O or K₂O, and an alkali halide such as LiF, NaF, CsF and KF. Among them, LiF, Li₂O and NaF are preferable. Examples of the alkaline earth metal compound include BaO, SrO, CaO, and mixtures thereof such as BaₓSr₁₋ₓO (0<x<1) and BaₓCa₁₋ₓO (0<x<1). Among them, BaO, SrO and CaO are preferable. Examples of the rare earth metal compound include YbF₃, ScF₃, ScO₃, Y₂O₃, Ce₂O₃, GdF₃ and TbF₃. Among these, YbF₃, ScF₃ and TbF₃ are preferable.

The alkali metal complexes, the alkaline earth metal complexes and the rare earth metal complexes are not particularly limited as long as they contain, as a metal ion, at least one of alkali metal ions, alkaline earth metal ions, and rare earth metal ions. Meanwhile, preferred examples of the ligand include, but are not limited to, quinolinol, benzoquinolinol, acridinol, phenanthridinol, hydroxyphenyloxazole, hydroxyphenylthiazole, hydroxydiaryloxadiazole, hydroxydiarylthiadiazole, hydroxyphenylpyridine, hydroxyphenylbenzimidazole, hydroxybenzotriazole, hydroxyfluborane, bipyridyl, phenanthroline, phthalocyanine, porphyrin, cyclopentadiene, β-diketones, and azomethines.

Regarding the addition form of the electron-donating dopant, it is preferred that the electron-donating dopant be formed in a shape of a layer or an island in the interfacial region. A preferred method for the formation is a method in which an organic compound (a light emitting material or an electron-injecting material) for forming the interfacial region is deposited simultaneously with deposition of the electron-donating dopant by a resistant heating deposition method, thereby dispersing the electron-donating dopant in the organic compound.

In a case where the electron-donating dopant is formed into the shape of a layer, the light-emitting material or electron-injecting material which serves as an organic layer in the interface is formed into the shape of a layer. After that, a reductive dopant is solely deposited by the resistant heating deposition method to form a layer preferably having a thickness of from 0.1 nm to 15 nm. In a case where the electron-donating dopant is formed into the shape of an island, the emitting material or the electron-injecting material which serves as an organic layer in the interface is formed into the shape of an island. After that, the electron-donating dopant is solely deposited by the resistant heating deposition method to form an island preferably having a thickness of from 0.05 nm to 1 nm. As the electron-transporting material used in the electron-transporting layer other than a compound of the formula (I), an aromatic heterocyclic compound having one or more hetero atoms in the molecule may preferably be used. In particular, a nitrogen containing heterocyclic compound is preferable.

According to one embodiment, it is preferable that the electron-transporting layer comprises a nitrogen containing heterocyclic metal chelate.

According to the other embodiment, it is preferable that the electron-transporting layer comprises a substituted or unsubstituted nitrogen containing heterocyclic compound. Specific examples of preferred heterocyclic compounds for the electron-transporting layer are, 6-membered azine compounds; such as pyridine compounds, pyrimidine compounds, triazine compounds, pyrazine compounds, preferably pyrimidine compounds or triazine compounds; 6-membered fused azine compounds, such as quinolone compounds, isoquinoline compounds, quinoxaline compounds, quinazoline compounds, phenanthroline compounds, benzoquinoline compounds, benzoisoquinoline compounds, dibenzoquinoxaline compounds, preferably quinolone compounds, isoquinoline compounds, phenanthroline compounds; 5-membered heterocyclic compounds, such as imidazole compounds, oxazole compounds, oxadiazole compounds, triazole compounds, thiazole compounds, thiadiazole compounds; fused imidazole compounds, such as benzimidazole compounds, imidazopyridine compounds, naphthoimidazole compounds, benzimidazophenanthridine compounds, benzimidzobenzimidazole compounds, preferably benzimidazole compounds, imidazopyridine compounds or benzimidazophenanthridine compounds.

According to another embodiment, it is preferable the electron-transporting layer comprises a phosphine oxide compound represented as Arₚ₁Arₚ₂Arₚ₃P═O.

Arₚ₁ to Arₚ₃ are the substituents of phosphor atom and each independently represent substituted or unsubstituted above mentioned aryl group or substituted or unsubstituted above mentioned heterocyclic group.

According to another embodiment, it is preferable that the electron-transporting layer comprises aromatic hydrocarbon compounds. Specific examples of preferred aromatic hydrocarbon compounds for the electron-transporting layer are, oligo-phenylene compounds, naphthalene compounds, fluorene compounds, fluoranthenyl group, anthracene compounds, phenanthrene compounds, pyrene compounds, triphenylene compounds, benzanthracene compounds, chrysene compounds, benzphenanthrene compounds, naphthacene compounds, and benzochrysene compounds, preferably anthracene compounds, pyrene compounds and fluoranthene compounds.

(Cathode)

For the cathode, a metal, an alloy, an electrically conductive compound, and a mixture thereof, each having a small work function (specifically, a work function of 3.8 eV or less) are preferably used. Specific examples of a material for the cathode include an alkali metal such as lithium and cesium; an alkaline earth metal such as magnesium, calcium, and strontium; an alloy containing these metals (for example, magnesium-silver, aluminum-lithium); a rare earth metal such as europium and ytterbium; and an alloy containing a rare earth metal.

The cathode is usually formed by a vacuum vapor deposition or a sputtering method. Further, in the case of using a silver paste or the like, a coating method, an inkjet method, or the like can be employed.

Moreover, when the electron-injecting layer is provided, various electrically conductive materials such as aluminum, silver, ITO, graphene, indium oxide-tin oxide containing silicon or silicon oxide, selected independently from the work function, can be used to form a cathode. These electrically conductive materials are made into films using a sputtering method, an inkjet method, a spin coating method, or the like.

(Insulating layer)

In the organic EL device, pixel defects based on leakage or a short circuit are easily generated since an electric field is applied to a thin film. In order to prevent this, it is preferred to insert an insulating thin layer between a pair of electrodes. Examples of materials used in the insulating layer include aluminum oxide, lithium fluoride, lithium oxide, cesium fluoride, cesium oxide, magnesium oxide, magnesium fluoride, calcium oxide, calcium fluoride, aluminum nitride, titanium oxide, silicon oxide, germanium oxide, silicon nitride, boron nitride, molybdenum oxide, ruthenium oxide, and vanadium oxide. A mixture thereof may be used in the insulating layer, and a laminate of a plurality of layers that include these materials can be also used for the insulating layer.

(Spacing Layer)

A spacing layer is a layer provided between a fluorescent emitting layer and a phosphorescent emitting layer when a fluorescent emitting layer and a phosphorescent emitting layer are stacked in order to prevent diffusion of excitons generated in the phosphorescent emitting layer to the fluorescent emitting layer or in order to adjust the carrier balance. Further, the spacing layer can be provided between the plural phosphorescent emitting layers.

Since the spacing layer is provided between the emitting layers, the material used for the spacing layer is preferably a material having both electron-transporting capability and hole-transporting capability. In order to prevent diffusion of the triplet energy in adjacent phosphorescent emitting layers, it is preferred that the spacing layer have a triplet energy of 2.6 eV or more. As the material used for the spacing layer, the same materials as those used in the above-mentioned hole-transporting layer can be given.

(Electron-Blocking Layer, Hole-Blocking Layer, Exciton-Blocking Layer)

An electron-blocking layer, a hole-blocking layer, an exciton (triplet)-blocking layer, and the like may be provided in adjacent to the emitting layer.

The electron-blocking layer has a function of preventing leakage of electrons from the emitting layer to the hole-transporting layer. The hole-blocking layer has a function of preventing leakage of holes from the emitting layer to the electron-transporting layer. In order to improve hole blocking capability, a material having a deep HOMO level is preferably used. The exciton-blocking layer has a function of preventing diffusion of excitons generated in the emitting layer to the adjacent layers and confining the excitons within the emitting layer. In order to improve triplet block capability, a material having a high triplet level is preferably used.

(Method for Forming a Layer)

The method for forming each layer of the organic EL device of the invention is not particularly limited unless otherwise specified. A known film-forming method such as a dry film-forming method, a wet film-forming method or the like can be used. Specific examples of the dry film-forming method include a vacuum deposition method, a sputtering method, a plasma method, an ion plating method, and the like. Specific examples of the wet film-forming method include various coating methods such as a spin coating method, a dipping method, a flow coating method, an inkjet method, and the like.

(Film Thickness)

The film thickness of each layer of the organic EL device of the invention is not particularly limited unless otherwise specified. If the film thickness is too small, defects such as pinholes are likely to occur to make it difficult to obtain a sufficient luminance. If the film thickness is too large, a high driving voltage is required to be applied, leading to a lowering in efficiency. In this respect, the film thickness is preferably 5 nm to 10 μm, and more preferably 10 nm to 0.2 μm.

(Electronic Apparatus (Electronic Equipment))

The present invention further relates to an electronic equipment (electronic apparatus) comprising the organic electroluminescence device according to the present application. Examples of the electronic apparatus include display parts such as an organic EL panel module; display devices of television sets, mobile phones, smart phones, and personal computer, and the like; and emitting devices of a lighting device and a vehicle lighting device.

### EXAMPLES

Next, the invention will be explained in more detail in accordance with the following synthesis examples, examples, and comparative examples, which should not be construed as limiting the scope of the invention.

The percentages and ratios mentioned in the examples below—unless stated otherwise—are % by weight and weight ratios.

### I Synthesis Examples

All experiments are carried out in protective gas atmosphere.

### Compound 11

### Intermediate 11-1

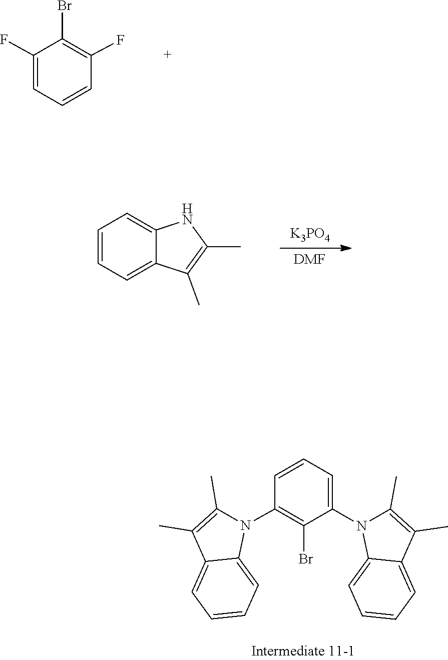

To 4.82 g (25.0 mmol) 1,3-difluoro-bromobenzene and 7.26 g(50 mmol) 2,3-dimethylindole in 50 ml water free DMF, 53.1 g (25 mmol) potassium phosphate tribasic was added. The reaction mixture was stirred at 150° C. for 18 h.

The reaction mixture was poured on water and the product was filtered off. The product was decocted in methanol, filtered with dichloromethane on silica gel and was decocted in iso-propanol. Yield 7.6 g (69%).

¹H-NMR (400 MHz, DMSO-d6) δ=7.78-7.84 (m, 1H), 7.68 (s, 2H), 7.48-7.53 (m, 2H), 7.03-7.11 (m, 4H), 6.85-6.89 (m, 1H), 6.77-6.81 (m, 1H), 2.28 (m, 6H), 2.18 (m, 6H).

### Compound 11

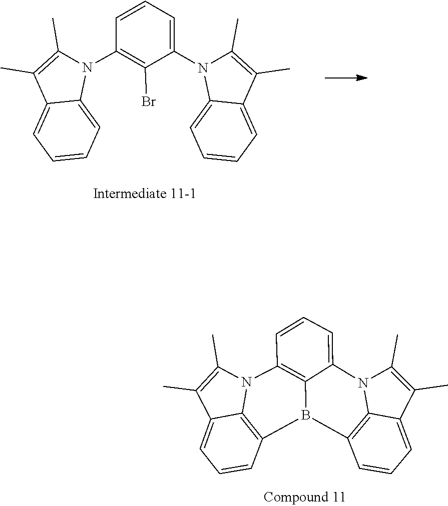

To 1.00 g (2.26 mmol) of Intermediate 11-1 in 10 ml water free tert-butyl benzene, 2.65 ml tert-butyl lithium in pentane (1.7 M solution in pentane) was added at 0° C. under argon. The reaction mixture was stirred at 60° C. for 3 h under argon. The reaction mixture was cooled to −50° C. and 1.13 g (4.51 mmol) bromo tribromide was added. The reaction mixture was stirred at 25° C. for 30 min. The reaction mixture was cooled again to 0° C. and 580 mg (4.47 mmol) N,N-di-isopropyl-ethylamine was added. The reaction was then heated at 120° C. for 12 h under argon.

The reaction mixture was poured on methanol and the product was filtered off. The product was filtered on silica gel with dichloromethane. Isopropanol was added and the dichloromethane was distilled off. The precipitated product was filtered off. Yield 130 mg (15 ¹H-NMR (400 MHz, CD₂Cl₂) δ=8.78 (dd, 2H), 7.97 (d, 2H), 7.88 (dd, 2H), 7.78 (dd, 1H), 7.56 (t, 2H), 2.97 (s, 6H), 2.44 (s, 6H).

### Compound 2

### Intermediate 2-1

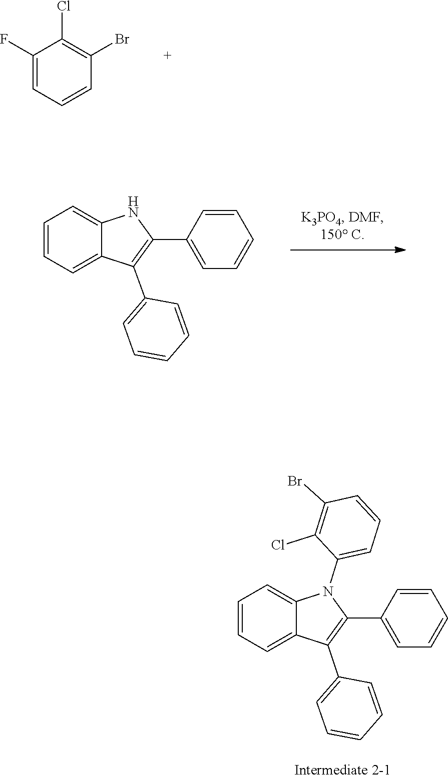 [SMILES: Fc1cccc(Br)c1Cl ; c1ccc(-c2[nH]c3ccccc3c2-c2ccccc2)cc1 ; Clc1c(Br)cccc1-n1c(-c2ccccc2)c(-c2ccccc2)c2ccccc21]

To 20.9 g (100 mmol) 1-bromo-2-chloro-3-fluoro-benzene and 13.5 g (50 mmol) 2,3-diphenyl-1H-indole, 53 g (250 mmol) potassium phosphate tribasic was added. 100 ml water free DMF was added. The reaction mixture was stirred at 150° C. under nitrogen for 18 h. The reaction mixture was poured on water and was extracted with ethyl acetate. The organic phase was dried with magnesium sulphate. The solvent was distilled off. The excess of 1-Bromo-2-chloro-3-fluoro-benzene was distilled off at 150° C. in high vacuum. The product was decocted in 100 ml methanol. Yield: 21.5 g (94%)

¹H-NMR (300 MHz, CD₂Cl₂) δ=7.81-7.87 (m, 1H), 7.74 (dd, 1H), 7.36-7.47 (m, 4H), 7.21-7.34 (m, 10H), 7.02-7.07 (m, 1H).

### Intermediate 2-2

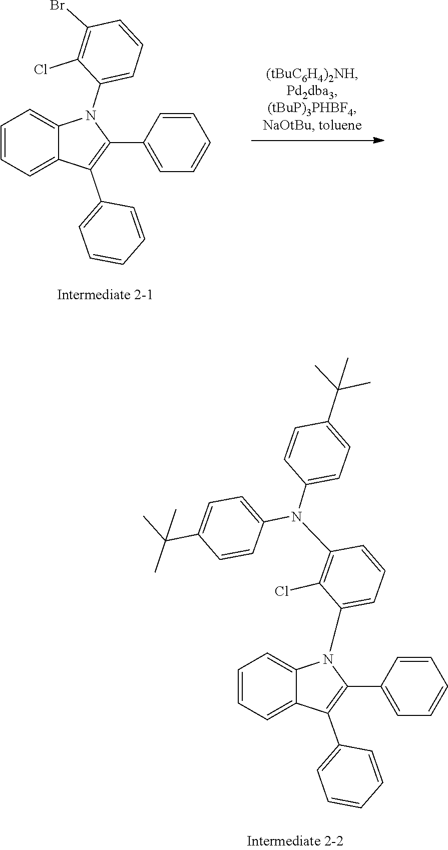 [SMILES: Clc1c(Br)cccc1-n1c(-c2ccccc2)c(-c2ccccc2)c2ccccc21 ; CC(C)(C)c1ccc(N(c2ccc(C(C)(C)C)cc2)c2cccc(-n3c(-c4ccccc4)c(-c4ccccc4)c4ccccc43)c2Cl)cc1]

8.80 g (19.2 mmol) Intermediate 2-1, 5.94 g (21.1 mmol) bis(4-(tert-butyl)phenyl)amine and 2.58 g (26.9 mmol) sodium tert-butoxide in 90 ml water free toluene were degassed with argon. 280 mg (0.480 mmol) (9,9-dimethyl-9H-xanthene-4,5-diyl)bis(diphenylphosphane) and 220 mg (0.240 mmol) Tris(dibenzylideneacetone)dipalladium(0) Pd₂(dba)₃ were added. The reaction mixture was degassed with argon. The reaction mixture was stirred for 18 h at 130° C. under argon.

300 mg sodium cyanide was added and the reaction mixture was stirred for 1 h at 90° C. Toluene was added and the organic phase was washed with water. The organic phase was dried with magnesium sulfate and the solvent was removed in vacuum.

The product was dissolved in dichloromethane, isopropanol was added and the dichloromethane was distilled off. Yield 8.6 g (68%).

¹H-NMR (300 MHz, CD₂Cl₂)⋅=7.81-7.85 (m, 1H), 7.12-7.44 (m, 21H), 6.76-6.77 (m, 3H), 1.33 (s, 18H).

### Compound 2

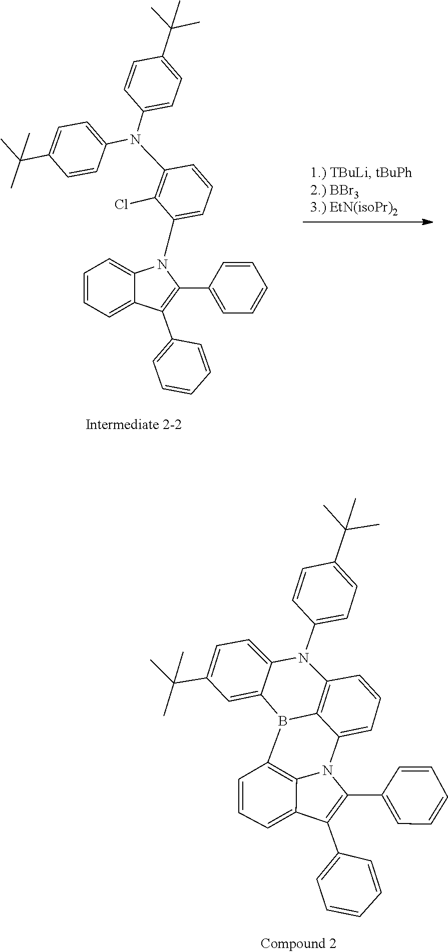 [SMILES: CC(C)(C)c1ccc(N(c2ccc(C(C)(C)C)cc2)c2cccc(-n3c(-c4ccccc4)c(-c4ccccc4)c4ccccc43)c2Cl)cc1 ; CC(C)(C)c1ccc(N2c3ccc(C(C)(C)C)cc3B3c4c2cccc4-n2c(-c4ccccc4)c(-c4ccccc4)c4cccc3c42)cc1]

To a solution of 10.0 g (15.2 mmol) Intermediate 2-2 in 200 ml water free tert-butyl benzene, 16.0 ml (30.3 mmol) tert-butyl lithium (1.9 M in pentane) were added at 0° C. under argon. The reaction mixture was stirred at 60° C. for 2.5 h under argon. To the reaction mixture 7.60 g (30.3 mmol) boron tribromide was added at 0° C. under argon. The reaction temperature was raised to 25° C. for 30 min. 3.92 g (30.3 mmol)N-ethyl-N-isopropyl propan-2-amine was added at 0° C. under argon. After 30 min the reaction mixture was stirred at 125° C. for 5 d under argon.

50 ml of a 10% sodium acetate solution was added and the precipitate was filtered off. The product was dissolved in toluene and was filtered on silica gel. The product was crystalized from 50 ml heptane and 5 ml toluene. Yield 450 mg (5%).

¹H-NMR (300 MHz, CD₂Cl₂) δ=9.08 (d, 1H), 8.84 (dd, 1H), 8.00 (dd, 1H), 7.72-7.78 (m, 2H), 7.70-7.68 (m, 2H), 7.45-7.56 (m, 5H), 7.28-7-41 (m, 7H), 7.13 (t, 1H), 6.81 (d, 1), 6.65 (d, 1H), 6.41 (d, 1H), 1.54 (s, 9H), 1.50 (s, 9H).

### Compound 4

### Intermediate 4-1

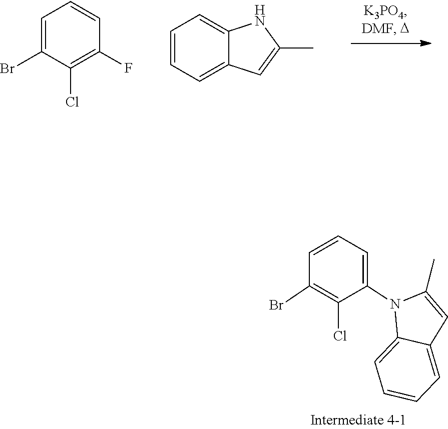

To 63.9 g (305 mmol) 1-bromo-2-chloro-3-fluorobenzene and 20 g (152 mml) 2-methyl-1H-indole in 200 ml water free DMF, 162 g (762 mmol) potassium phosphate tribasic were added under nitrogen. The reaction mixture was stirred at 175° C. for 18 h.

The solids were filtered off and the solvent was removed in vacuum. The product was crystallized from ethanol. The product filtered on silica gel with dichloromethane. Ethanol was added and the dichloromethane was distilled off. The crystallized product was filtered off.

Yield 38.5 g (79%).

¹H-NMR (300 MHz, CD₂Cl₂) δ=7.87 (dd, 1H), 7.56-7.62 (m, 1H), 7.36-7.45 (m, 2H), 7.08-7.17 (m, 2H), 6.82-6.88 (m, 1H), 6.46-6.47 (m, 1H), 2.25 (s, 3H).

### Intermediate 4-2

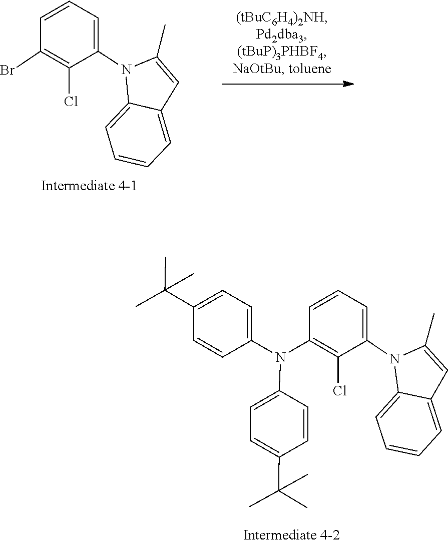 [SMILES: Cc1cc2ccccc2n1-c1cccc(Br)c1Cl ; Cc1cc2ccccc2n1-c1cccc(N(c2ccc(C(C)(C)C)cc2)c2ccc(C(C)(C)C)cc2)c1Cl]

To 35.0 g (109 mmol) of Intermediate 4-1 and 33.8 g (120 mmol) bis(4-(tert-butyl)phenyl)amine in 350 ml water free toluene, 14.7 g (153 mmol) sodium tert-butoxide were added. The reaction mixture was degassed with argon. 1.58 g (2.73 mmol) (9,9-dimethyl-9H-xanthene-4,5-diyl)bis(diphenylphosphane) and 1.25 g (1.37 mmol) tris(dibenzylideneacetone)dipalladium(0) Pd₂(dba)₃ were added. The reaction mixture was degassed with argon. The reaction mixture was stirred at 130° C. under argon for 2 h. 300 mg sodium cyanide in 100 ml water and 5 g charcoal were added and the reaction mixture was stirred for 1 h at 100° C. The organic phase was separated, washed with water and hydrochloric acid and dried with magnesium sulfate. The product was filtered on silica gel with ethyl acetate/dichloromethane 1/5. The product was crystallized from acetone. Yield 37.5 g (66%).

¹H-NMR (300 MHz, CD₂Cl₂) δ=7.54-7.60 (m, 1H), 7.41-7.50 (m, 2H), 7.30-7.35 (m, 5H), 7.09-7.15 (m, 2H), 6.91-7.00 (m, 5H), 6.44 (t, 1H), 2.26 (s, 3H), 1.34 (s, 18H).

### Intermediate 4-3

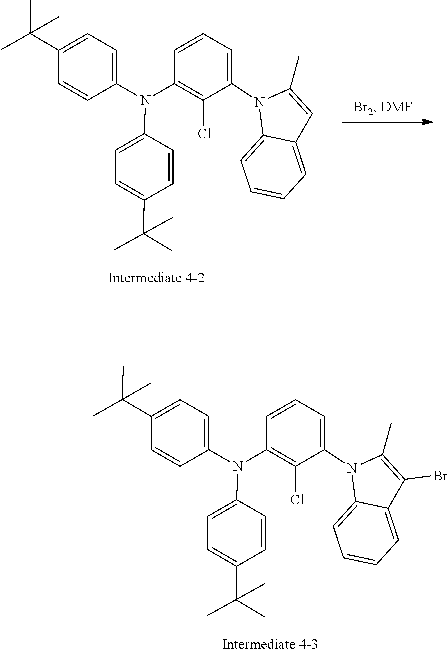 [SMILES: Cc1cc2ccccc2n1-c1cccc(N(c2ccc(C(C)(C)C)cc2)c2ccc(C(C)(C)C)cc2)c1Cl ; Cc1c(Br)c2ccccc2n1-c1cccc(N(c2ccc(C(C)(C)C)cc2)c2ccc(C(C)(C)C)cc2)c1Cl]

7.00 g (13.4 mmol) of Intermediate 4-2 were dissolved in dichloromethane under nitrogen. The reaction mixture was cooled to −50° C. and bromine 2.15 g (13.4 mmol) was added slowly. The reaction mixture was stirred for 1 h. 2 g sodium dithionite (Na₂S₂O₄) was added and the reaction mixture was stirred at 0° C. for 15 min. The solids were filtered off and washed with dichloromethane. The solvent was removed in vacuum. Column chromatography on silica gel with heptane, than heptane/ethyl acetate 1/1 gave the product.

Yield 5.8 g (72%).

¹H-NMR (300 MHz, CD₂Cl₂) δ=7.16-7.56 (m, 10H), 6.90-7.00 (m, 5H), 2.27 (s, 3H), 1.34 (s, 18H).

### Intermediate 4-4

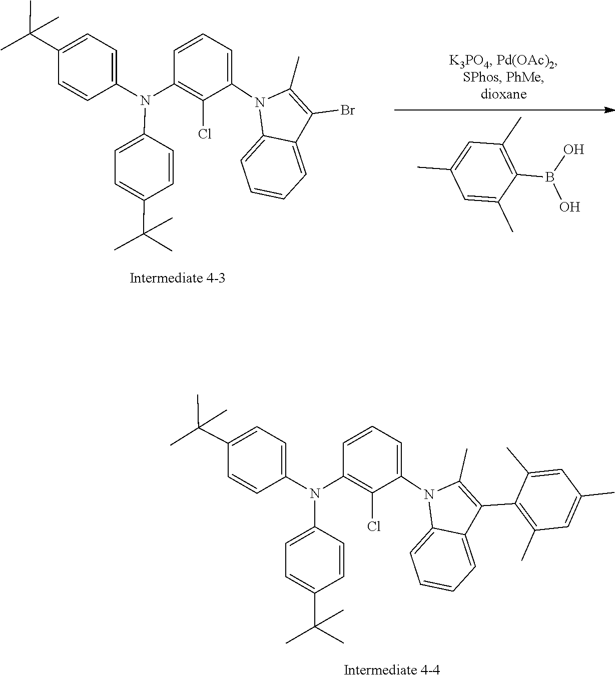 [SMILES: Cc1c(Br)c2ccccc2n1-c1cccc(N(c2ccc(C(C)(C)C)cc2)c2ccc(C(C)(C)C)cc2)c1Cl ; Cc1cc(C)c(B(O)O)c(C)c1 ; Cc1cc(C)c(-c2c(C)n(-c3cccc(N(c4ccc(C(C)(C)C)cc4)c4ccc(C(C)(C)C)cc4)c3Cl)c3ccccc23)c(C)c1]

7.57 g (12.6 mmol) of Intermediate 4-3, 4.14 g (25.2 mmol) mesityl boronic acid, 5.23 g (37.8 mmol) potassium carbonate and 0.203 g (0.631 mmol) tetra-butyl-ammonium bromide in 75 ml toluene, 30 ml ethanol and 15 ml water were degassed with argon. 0.113 g (0.505 mmol) diacetoxypalladium and 410 mg (1.01 mmol) dicyclohexyl(2′,6′-dimethoxy-[1,1′-biphenyl]-2-yl)phosphane were added. The reaction mixture was degassed with argon. The reaction mixture was stirred at 80° C. for 3 h. 200 mg sodium cyanide in 50 ml water were added and the reaction mixture was stirred for 1 h at 100° C.

The organic phase was separated and washed with water. The organic phase was dried with magnesium sulfate and the solvent was removed in vacuum. Column chromatography on silica gel with heptane and then heptane/ethyl acetate 1/1 gave the product. Yield 7.2 g (89

### Intermediate 4-5

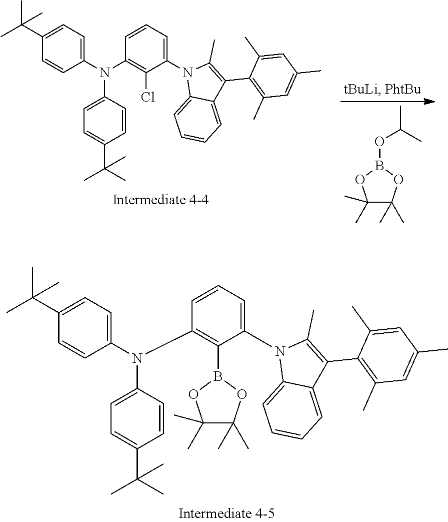

5.00 g (7.82 mmol) of Intermediate 4-4 were dissolved in water free tert-butyl benzene under nitrogen. The reaction mixture was cooled to 0° C. and 8.23 ml (15.6 mmol) tert-butyl lithium (1.9 M in pentane) were added. The reaction mixture was heated to 60° C. under nitrogen for 1 h. The reaction mixture was cooled to −20° C. and 2.91 g (15.6 mmol) 4,4,5,5-tetramethyl-2-(1-methylethoxy)-1,3,2-dioxaborolane were added. The reaction temperature was raised to 25° C. The reaction mixture was stirred at 25° C. for 90 min.

The reaction mixture was poured on a sodium acetate solution and was extracted with toluene. The solvent was removed in vacuum.

The product was dissolved in dichloromethane, 100 ml methanol was added and the dichloromethane was distilled off. The product was filtered off. Yield 2.5 g (44%).

### Compound 4

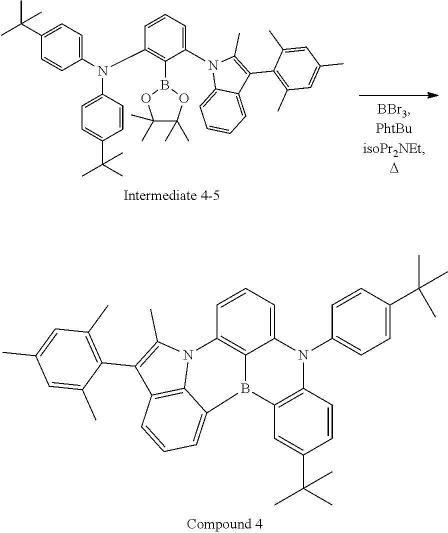

2.47 g (3.38 mmol) of Intermediate 4-5 were dissolved in 25 ml water free tert-butyl benzene. 960 ml (10.1 mmol) tribromo borane were added under nitrogen at 25° C. 1.80 ml (10.1 mml) N-ethyl-N,N-diisopropyl amine were added at 25° C. under nitrogen. After 10 min the reaction mixture was stirred at 125° C. The reaction mixture was stirred for 30 min, then the reaction temperature was increased to 165° C. The reaction mixture was stirred for 18 h at 165° C. under nitrogen.

The reaction mixture was poured on a sodium acetate solution and was extracted with toluene. The solvent was removed in vacuum. Column chromatography on silica gel with heptane, later heptane/toluene 1/1.

The product was dissolved in dichloromethane, 30 ml methanol were added and the dichloromethane was distilled off. The product was filtered off. The product was dissolved again in dichloromethane and 20 ml acetonitrile was added. The dichloromethane was distilled off and the product was filtered off. Yield 450 mg (21%).

¹H-NMR (300 MHz, CD₂Cl₂) δ=9.10 (d, 1H), 8.77 (d, 1H), 7.77-7.85 (m, 3H), 7.33-7.64 (m, 6H), 7.10 (s, 2H), 6.82 (d, 1H), 6.53 (d, 1H), 2.77 (s, 3H), 2.44 (s, 3H), 2.11 (s, 6H), 1.53 (s, 9H), 1.52 (s, 9H).

### Compound 1

### Intermediate 1-1

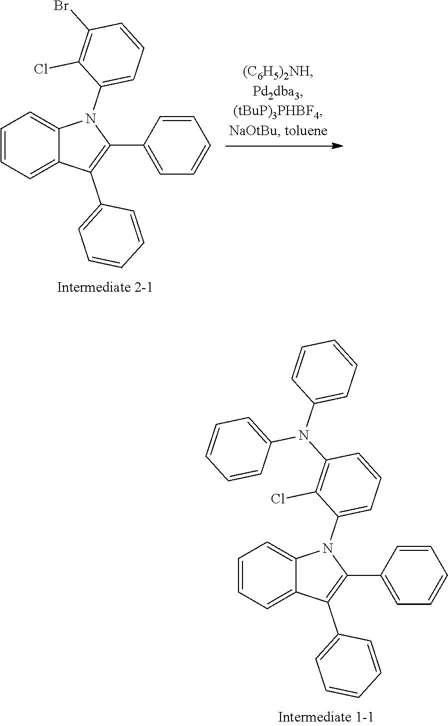 [SMILES: Clc1c(Br)cccc1-n1c(-c2ccccc2)c(-c2ccccc2)c2ccccc21 ; Clc1c(N(c2ccccc2)c2ccccc2)cccc1-n1c(-c2ccccc2)c(-c2ccccc2)c2ccccc21]

5.00 g (10.9 mmol) of Intermediate 2-1, 2.03 g (12.0 mmol) diphenylamine and 1.47 g (15.3 mmol) sodium tert-butoxide in 50 ml water free xylene were degassed with argon. 148 mg (0.272 mmol) (9,9-dimethyl-9H-xanthene-4,5-diyl)bis(diphenylphosphane) and 125 mg (0.136 mmol) Tris(dibenzylideneacetone)dipalladium(0) Pd₂(dba)₃ were added. The reaction mixture was degassed with argon. The reaction mixture was stirred for 18 h at 120° C. under argon. 300 mg sodium cyanide in 20 ml water were added and the reaction mixture was stirred for 1 h at 90° C. Toluene was added and the organic phase was washed with water. The organic phase was dried with magnesium sulfate and the solvent was removed in vacuum. Yield 4.38 g (67%).

¹H-NMR (400 MHz, CD₂Cl₂) δ=7.80-7.82 (m, 1H), 7.18-7.45 (m, 19H), 7.12-7.15 (m, 1H), 7.00-7.05 (m, 2H), 6.87-6.90 (m, 4H).

### Intermediate 1-2

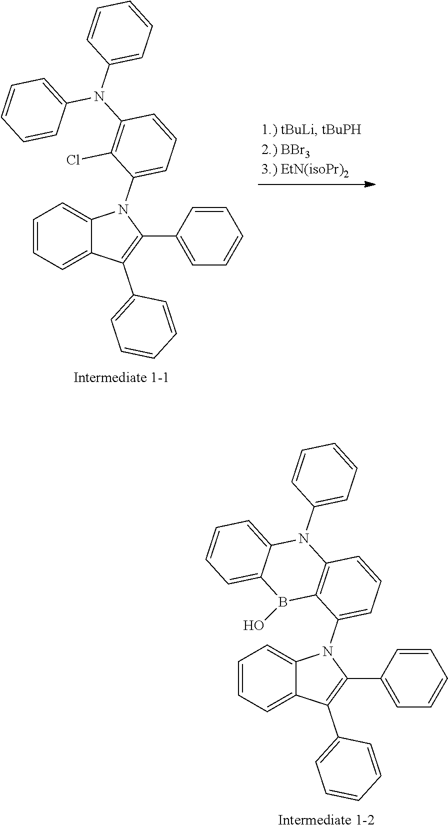 [SMILES: Clc1c(N(c2ccccc2)c2ccccc2)cccc1-n1c(-c2ccccc2)c(-c2ccccc2)c2ccccc21 ; OB1c2ccccc2N(c2ccccc2)c2cccc(-n3c(-c4ccccc4)c(-c4ccccc4)c4ccccc43)c21]

To a solution of 3.0 g (5.48 mmol) of Intermediate 1-1 in 150 ml water free tert-butyl benzene, 6.45 ml (11.0 mmol) tert-butyl lithium (1.7 M in pentane) were added at 0° C. under argon. The reaction mixture was stirred at 60° C. for 1.5 h under argon. To the reaction mixture 1.1 ml (11.0 mmol) boron tribromide were added at −30° C. under argon. The reaction temperature was raised to 25° C. for 10 min. 1.9 ml (11.0 mmol)N-ethyl-N-isopropyl propan-2-amine were added at 0° C. under argon. After 30 min the reaction mixture was stirred at 125° C. for 18 h under argon.

The reaction mixture was poured on a 10% sodium acetate solution and the organic phase was separated. The organic phase was washed with water and dried with magnesium sulfate. The solvent was removed in vacuum. Column chromatography on silica gel with heptane and then heptane ethyl acetate 1/1 gave the product. Yield 1.6 g (54%).

### Compound 1

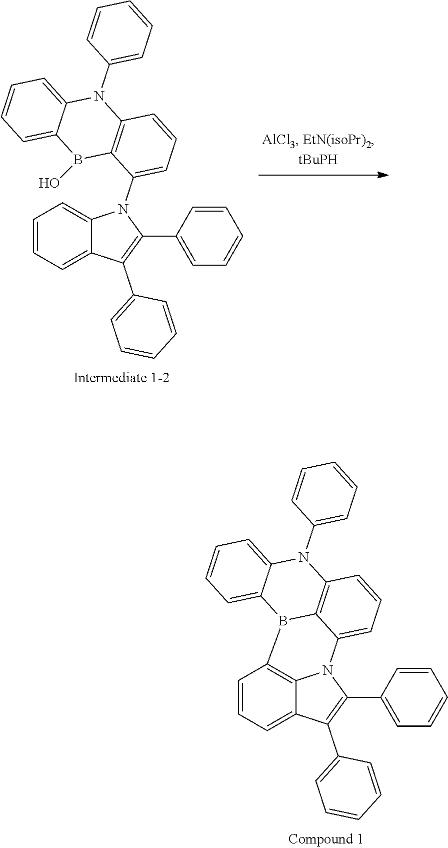 [SMILES: OB1c2ccccc2N(c2ccccc2)c2cccc(-n3c(-c4ccccc4)c(-c4ccccc4)c4ccccc43)c21 ; c1ccc(-c2c(-c3ccccc3)n3c4c(cccc24)B2c4ccccc4N(c4ccccc4)c4cccc-3c42)cc1]

To 500 mg (0.929 mmol) of Intermediate 1-2 in 10 ml water free tert-butyl benzene, 1.24 g (9.29 mmol) aluminum trichloride and 600 mg (4.64 mmol)N-ethyl-N-isopropyl propan-2-amine were added. The reaction mixture was stirred at 80° C. for 2 h under argon. The reaction mixture was poured on water and was extracted with toluene. The organic phase was washed with water and the organic phase was dried with magnesium sulfate.

The product was dissolved in dichloromethane and ethanol was added. The dichloromethane was removed in vacuum. The product was filtered off. This procedure was repeated once.

The product was dissolved in dichloromethane and acetonitrile was added. The dichloromethane was removed in vacuum. The product was filtered off.

The product was dissolved in dichloromethane and heptane was added. The dichloromethane was removed in vacuum. The product was filtered off. Yield: 160 mg (33

¹H-NMR (400 MHz, CD₂Cl₂) δ=9.09 (dd, 1H), 8.85 (dd, 1H), 8.00 (d, 1H), 7.73-7.80 (m, 2H), 7.63-7.70 (m, 2H), 7.27-7.56 (m, 14H), 7.15 (t, 1H), 6.83 (dd, 1H), 6.67 (d, 1H), 6.37 (d, 1H).

### Compound 3

### Intermediate 3-1

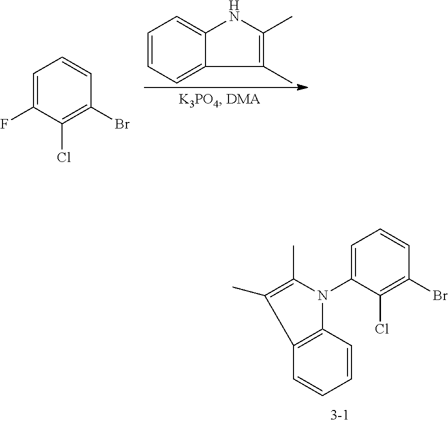 [SMILES: Fc1cccc(Br)c1Cl ; Cc1[nH]c2ccccc2c1C ; Cc1c(C)n(-c2cccc(Br)c2Cl)c2ccccc12]

9.56 g (65.8 mmol) of 2,3-dimethyl-1H-indole, 27.6 g (132 mmol) of 1-bromo-2-chloro-3-fluorobenzene, and 69.9 g (329 mmol) of potassium phosphate were suspended in 440 ml of N,N-dimethylacetamide, followed by heating at 140° C. during 9 hours. The suspension was filtered, the solid washed with toluene, and the collected eluents concentrated under vacuum. The resulting oil was diluted with 150 ml of ethanol and 100 ml of water, and the resulting suspension filtered. The solid was suspended in 190 ml of ethanol and heated under reflux overnight. The suspension was filtered, washed with a small amount of ethanol. The solid was further recrystallized from 170 ml of 2-propanol, giving Intermediate 3-1 as a white solid (yield: 15.9 g (72%)).

¹H-NMR (400 MHz, CDCl₃): δ=7.80 (dd, 1H), 7.57 (m, 1H), 7.37-7.29 (m, 2H), 7.19-7.08 (m, 2H), 6.84 (m, 1H), 2.34 (s, 3H), 2.16 (s, 3H).

### Intermediate 3-2

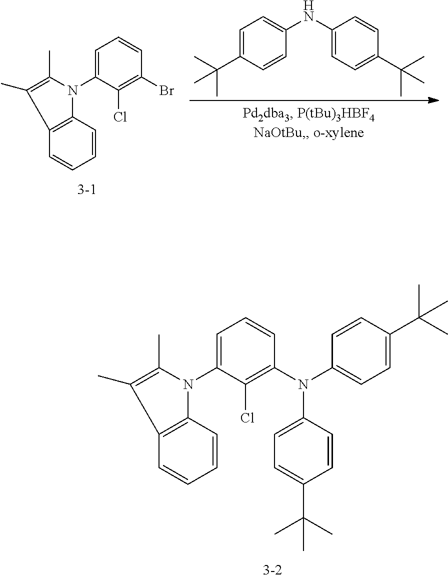

14.3 g (42.6 mmol) of Intermediate 3-1, 13.2 g (46.9 mmol) of bis(4-(tert-butyl)phenyl)amine, 0.78 g (0.85 mmol) of tris(dibenzylideneacetone)dipalladium(0), 0.99 g (3.41 mmol) of tri-tert-butylphosphonium tetrafluoroborate, and 1.40 g (59.7 mmol) of sodium tert-butoxide were suspended in 170 ml of o-xylene. The suspension was three times evacuated and backfilled with argon and heated at 110° C. during 2.5 hours. The dark suspension was cooled down and 70 g of silica gel were added. The suspension was concentrated under vacuum, the solid further purified by chromatography (silica gel, heptane/toluene 9:1), and the product fractions concentrated under vacuum. The white foam was dissolved in the minimum amount of dichloromethane, and diluted with acetonitrile. The solution was concentrated under vacuum until a suspension was formed. The suspension was filtered and the solid washed with acetonitrile, giving Intermediate 3-2 as a white solid (yield: 14.9 g (65%)).

¹H-NMR (400 MHz, DMSO-d₆): δ=7.59 (t, 1H), 7.52-7.46 (m, 1H), 7.46-7.38 (m, 2H), 7.35-7.28 (m, 4H), 7.09-7.02 (m, 2H), 6.91-6.84 (m, 4H), 6.83-6.77 (m, 1H), 2.26 (s, 3H), 2.11 (s, 3H), 1.27 (s, 18H).

### Intermediate 3-3

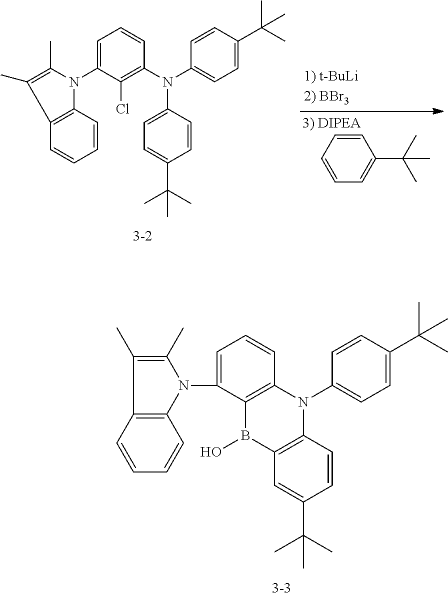

5.10 g (9.55 mmol) of Intermediate 3-2 were dissolved in 76 ml of water-free tert-butyl benzene. 10.0 ml of tert-butyl lithium (1.9 M in pentane) were slowly added at −6° C. The yellow solution was heated up to 70° C. and pentane distilled off. The light brown solution was cooled down to −70° C. and 1.8 ml (19 mmol) of tribromoborane were slowly added. The reaction mixture was stirred at room temperature during 15 minutes and cooled down to 0° C. 3.3 ml (19 mmol) of N,N-diisopropylethylamine were added and the reaction mixture heated up to 145° C. during 6 hours. The brown suspension was poured into a mixture of 10% aqueous sodium acetate solution and toluene, and the organic layer separated. The aqueous layer was extracted twice with toluene. The combined organic layers were washed three times with water and once with brine, dried over sodium sulfate, and concentrated under vacuum. The brown oil was purified by MPLC with the CombiFlash Companion (silica gel, heptane/0-80% gradient of toluene), giving Intermediate 3-3 as a yellow solid (yield: 1.85 g (37%)).

¹H-NMR (400 MHz, CDCl₃): δ=8.14 (d, 1H), 7.76-7.69 (m, 2H), 7.63 (d, 1H), 7.54-7.43 (m, 2H), 7.35-7.29 (m, 2H), 7.21-7.15 (m, 1H), 7.13-7.07 (m, 1H), 6,96 (d, 1H), 6.91-6.84 (m, 2H), 6.61 (d, 1H), 4.78 (s, 1H), 2.42 (s, 3H), 2.21 (s, 3H), 1.50 (s, 9H), 1.33 (s, 9H).

### Compound 3

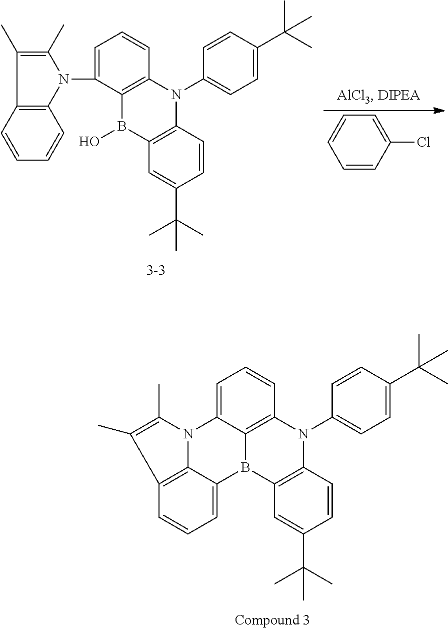

2.35 g (4.46 mmol) of Intermediate 3-3 were dissolved in 22 ml of chlorobenzene. 5.95 g (44.6 mmol) of aluminium chloride and 3.9 ml (22.3 mmol) of N,N-diisopropylethylamine were slowly added, followed by heating at 120° C. during 12 hours. The reaction mixture was cooled down to room temperature and poured into an ice-water mixture, followed by extraction with toluene (three times). The combined organic layers were washed with brine, dried over sodium sulfate, and concentrated under vacuum. The brown resin was purified by MPLC with the CombiFlash Companion (silica gel, heptane/0-45% gradient of toluene). The orange solid was dissolved in dichloromethane and diluted with a 1:1 mixture of 15 ml n-butyl acetate and 2-propanol. The solution was concentrated under vacuum until a suspension formed. The suspension was filtered and the solid dissolved in dichloromethane and diluted with acetonitrile. The solution was concentrated under vacuum until a suspension formed, giving Compound 3 as a yellow solid (yield: 783 mg (34%)).

¹H-NMR (400 MHz, CD₂Cl₂): δ=9.06 (d, 1H), 8.74 (d, 1H), 7.87 (d, 1H), 7.82-7.73 (m, 3H), 7.64-7.51 (m, 3H), 7.37-7.29 (m, 2H), 6.78 (d, 1H), 6.48 (d, 1H), 2.98 (s, 3H), 2.46 (s, 3H), 1.52 (s, 9H), 1.51 (s, 9H).

### Compound 5

### Intermediate 5-1

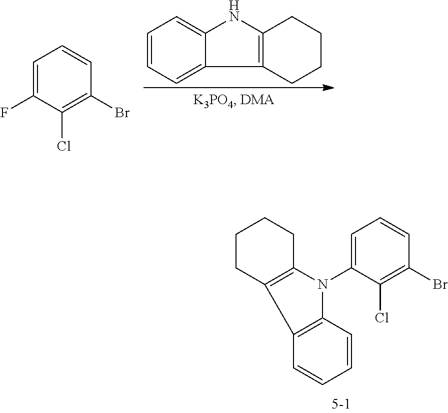 [SMILES: Fc1cccc(Br)c1Cl ; c1ccc2c3c([nH]c2c1)CCCC3 ; Clc1c(Br)cccc1-n1c2c(c3ccccc31)CCCC2]

15.9 g (92.6 mmol) of 2,3,4,9-tetrahydro-1H-carbazole, 38.8 g (185 mmol) of 1-bromo-2-chloro-3-fluorobenzene, and 78.6 g (370 mmol) of potassium phosphate were suspended in 460 ml of N,N-dimethylacetamide, followed by heating at 130° C. during 19 hours. The suspension was filtered, the solid washed with toluene, and the collected eluents concentrated under vacuum. The resulting oil was further purified by chromatography (silica gel, heptane), giving Intermediate 5-1 as a colorless oil (yield: 31.5 g (94%)).

¹H-NMR (400 MHz, CDCl₃): δ=7.78 (dd, 1H), 7.58-7.53 (m, 1H), 7.35 (dd, 1H), 7.31 (d, 1H), 7.19-7.09 (m, 2H), 6.94-6.87 (m, 1H), 2.91-2.75 (m, 2H), 2.56-2.39 (m, 2H), 2.03-1.83 (m, 4H).

### Intermediate 5-2

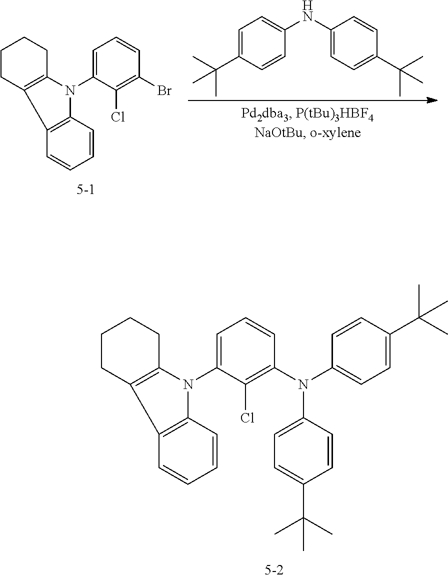

31.5 g (87.3 mmol) of Intermediate 5-1, 27.0 g (96.1 mmol) of bis(4-(tert-butyl)phenyl)amine, 1.60 g (1.75 mmol) of tris(dibenzylideneacetone)dipalladium(0), 2.03 g (6.99 mmol) of tri-tert-butylphosphonium tetrafluoroborate, and 11.8 g (122 mmol) of sodium tert-butoxide were suspended in 290 ml of o-xylene. The suspension was three times evacuated and backfilled with argon and heated at 115° C. during 2 hours. The reaction mixture was cooled down and 70 g of silica gel were added. The suspension was concentrated under vacuum, the solid further purified by chromatography (silica gel, heptane/toluene 9:1), and the product fractions concentrated under vacuum. The solid was dissolved in dichloromethane and diluted with ethanol. The solution was concentrated under vacuum until a suspension formed. The suspension was filtered and the solid washed with a small amount of ethanol. The solid was dissolved in dichloromethane and diluted with acetonitrile. The solution was concentrated under vacuum until a suspension formed, giving Intermediate 5-2 as a white solid (yield: 29.1 g (59%)).

¹H-NMR (400 MHz, DMSO-d₆): δ=7.57 (t, 1H), 7.49-7.39 (m, 3H), 7.36-7.29 (m, 4H), 7.09-7.02 (m, 2H), 6.90-6.80 (m, 5H), 2.76-2.65 (m, 2H), 2.41-2.27 (m, 2H), 1.93-1.76 (m, 4H), 1.27 (s, 18H).

### Compound 5

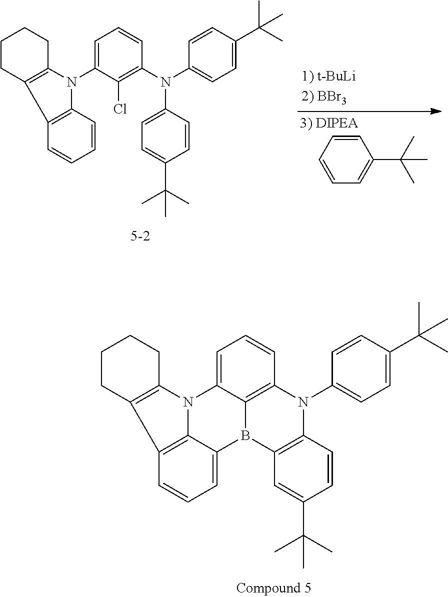

4.80 g (8.55 mmol) of Intermediate 5-2 were dissolved in 68 ml of water-free tert-butyl benzene. 9.0 ml of tert-butyl lithium (1.9 M in pentane) were slowly added at −6° C. The yellow solution was heated up to 70° C. and pentane distilled off. The light brown solution was cooled down to −70° C. and 1.6 ml (17.1 mmol) of tribromoborane were slowly added. The reaction mixture was stirred at room temperature during 15 minutes and cooled down to 0° C. 3.0 ml (17.1 mmol) of N,N-diisopropylethylamine were added and the reaction mixture heated up to 145° C. during 48 hours. The brown suspension was poured into a mixture of 10% aqueous sodium acetate solution and toluene. The aqueous layer was separated and extracted twice with toluene. The combined organic layers were washed three times with water and once with brine, then dried over sodium sulfate, and concentrated under vacuum. The brown oil was purified by MPLC with the CombiFlash Companion (silica gel, heptane/0-25% gradient of toluene). The resulting solid was dissolved in dichloromethane and diluted with heptane. The solution was concentrated under vacuum until a suspension formed. The suspension was filtered and the solid dissolved in dichloromethane and diluted with acetonitrile. The solution was concentrated until a suspension formed. The suspension was filtered and precipitation was repeated three times by using combinations of dichloromethane with 2-propanol or heptane, giving Compound 5 as a yellow solid (yield: 1.09 g (24%)).

¹H-NMR (400 MHz, DMSO-d₆): δ=8.93 (d, 1H), 8.63 (dd, 1H), 7.89-7.77 (m, 3H), 7.72-7.52 (m, 4H), 7.41-7.33 (m, 2H), 6.65 (d, 1H), 6.34 (dd, 1H), 3.45-3.35 (m, 2H), 2.93-2.82 (m, 2H), 2.07-1.87 (m, 4H), 1.45 (s, 9H), 1.44 (s, 9H).

### Compound 9

### Intermediate 9-1

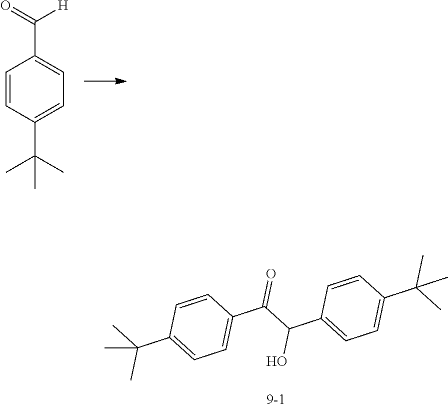 [SMILES: CC(C)(C)c1ccc(C=O)cc1 ; CC(C)(C)c1ccc(C(=O)C(O)c2ccc(C(C)(C)C)cc2)cc1]

To 250 g (1.54 mol) of 4-tert-butyl-benzaldehyde dissolved in 500 ml of ethanol, 12.5 g (193 mmol) of potassium cyanide in 50 ml of water were added. The reaction mixture was stirred at 100° C. under nitrogen. The reaction was completed after 5 hours. The product was filtered off at room temperature and was washed with water. The reaction mixture was dried. Yield: 170 g (68%) of Intermediate 9-1.

¹H-NMR (400 MHz, CDCl₃): δ=7.89-7.93 (m, 2H), 7.42-7.46 (m, 2H), 7.35-7.38 (m, 2H), 7.27-7.31 (m, 2H), (5.93 d, 1H), 4.53 (d, 1H), 1.32 (s, 9H), 1.29 (s, 9H).

### Intermediate 9-2

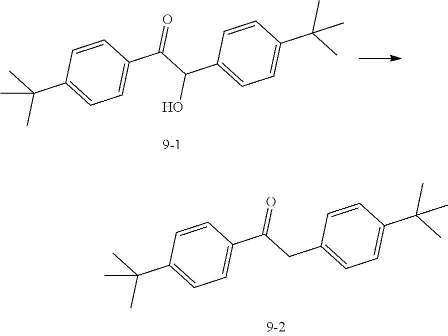

To 20.0 g (61.6 mmol) of Intermediate 9-1 in 40 ml acetic acid (99%) at 100° C., 8.06 g (123 mmol) zinc powder were added. The reaction was stirred at 100° C. for 19 hours under nitrogen. 100 ml of ethyl acetate were added and the solids were filtered off. 50 ml of water were added and ethyl acetate was distilled off. The product was filtered off and washed with water and methanol. Yield: 14.0 g (73%) of Intermediate 9-2.

¹H-NMR (400 MHz, CDCl₃): δ=7.97-8.01 (m, 2H), 7.48-7.51 (m, 2H), 7.35-7.38 (m, 2H), 7.21-7.25 (m, 2H), 4.26 (s, 2H), 1.26 (s, 9H), 1.33 (s, 9H).

### Intermediate 9-3

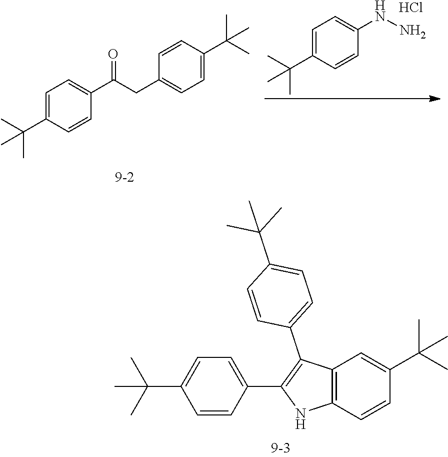

To 30.0 g (97.3 mmol) of Intermediate 9-2 and 20.5 g (102 mmol) of 4-tert-butylphenyihydrazine mono-hydrochloride in 200 ml of ethanol, 19.8 g (19.4 mmol) of sulfuric acid (95-97%) were added. The reaction mixture was stirred at 100° C. for 4 hours under nitrogen. The reaction mixture was cooled to 25° C. and the product was filtered off. The product was washed with water and ethanol. Column chromatography on silica gel with heptane and then heptane/toluene 1/1 gave Intermediate 9-3. Yield: 22.5 g (53%).

¹H-NMR (400 MHz, CDCl₃): δ=11.3 (s, 1H (NH)), 7.41-7.45 (m, 3H), 7.34-7.38 (m, 5H), 7.29-7.31 (m, 2H), 7.22 (dd, 1H), 1.34 (s, 9H), 1.31 (s, 9H), 1.29 (s, 9H).

### Intermediate 9-4

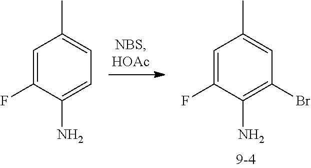

50.0 g (0.40 mol) of 2-fluoro-4-methylaniline were dissolved in 250 ml of acetic acid. 78.0 g (0.44 mol) of N-bromosuccinimide were added in small portions at a maximum temperature of 16° C. during 45 minutes, controlling the temperature with an ice-bath. The ice-bath was removed and stirring continued during 90 minutes. 400 ml of water and 500 ml of heptane were added and the resulting mixture washed with water (3×400 ml) and 10% aqueous sodium carbonate solution (200 ml). The organic phased was washed with water (2×300 ml), dried over sodium sulfate and concentrated under vacuum, giving Intermediate 9-4 as a light brown solid (yield: 76.1 g (93%)).

¹H-NMR (400 MHz, CD₂Cl₂): δ=7.10-7.06 (m, 1H), 6.87-6.81 (m, 1H), 4.11 (br. s, 2H), 2.26 (s, 3H).

### Intermediate 9-5

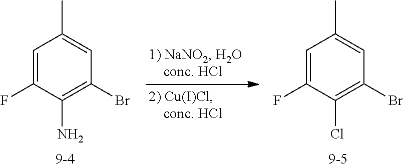

30.0 g (147 mmol) of Intermediate 9-4 were suspended in 250 ml of 37% hydrochloric acid solution. The suspension was dropwise treated with a solution of 12.2 g (176 mmol) of sodium nitrite in 70 ml of water at 3° C. during 30 minutes. The resulting cloudy solution was dropwise treated with a solution of 21.8 g (221 mmol) of copper(I) chloride in 100 ml of 37% hydrochloric acid at 3° C. during 40 minutes. The ice-bath was removed and the reaction mixture stirred during two hours, followed by the addition of 600 ml of water. The brown suspension was extracted with ethyl acetate and the organic phase washed with water (3×200 ml), followed by stirring with 150 ml of 10% aqueous ammonia solution during 10 minutes. The organic phase was separated and washed with water (2×200 ml), dried over sodium sulfate, and concentrated under vacuum. The product was further purified by MPLC with the CombiFlash Companion (silica gel, heptane/0-7% gradient of ethyl acetate), giving Intermediate 9-5 as an oil (yield: 18.5 g (56%)).

¹H-NMR (400 MHz, CD₂Cl₂): δ=7.32-7.28 (m, 1H), 7.01-6.95 (m, 1H), 2.35 (s, 3H).

### Intermediate 9-6

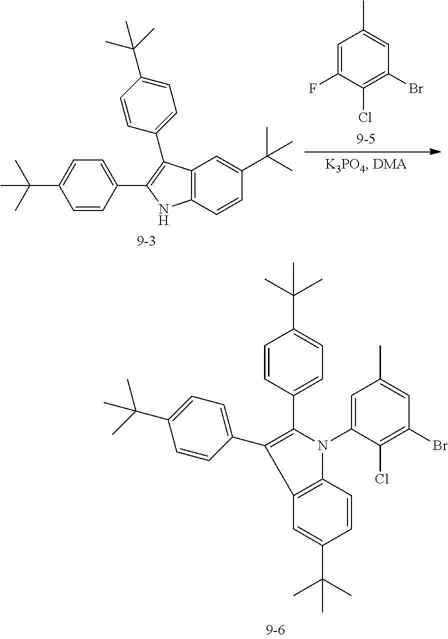

7.15 g (32.0 mmol) of Intermediate 9-5, 7.00 g (16.0 mmol) of Intermediate 9-3, and 17.0 g (80.0 mmol) of potassium phosphate were suspended in 80 ml of N,N-dimethylacetamide, followed by heating at 152° C. during 12 hours. The yellow suspension was poured into 200 ml of water, the suspension filtered, and the solid washed with 200 ml of water. The resulting solid was further suspended in 100 ml of ethanol, followed by filtration and additional washing with ethanol, giving Intermediate 9-6 as a white solid (yield: 8.10 g (79%)).

¹H-NMR (400 MHz, CD₂Cl₂): δ=7.77 (dd, 1H), 7.54 (dd, 1H), 7.45-7.30 (m, 5H), 7.26-7.21 (m, 2H), 7.15-7.10 (m, 2H), 7.07-7.03 (m, 1H), 6.93 (dd, 1H), 2.28 (s, 3H), 1.42 (s, 9H), 1.39 (s, 9H), 1.29 (s, 9H).

### Intermediate 9-7

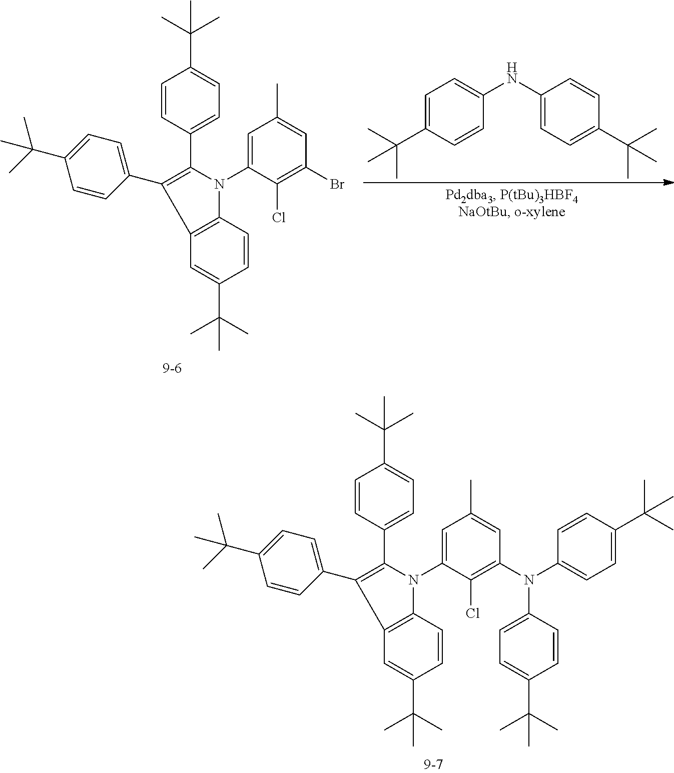

4.80 g (7.49 mmol) of Intermediate 9-6, 2.21 g (7.86 mmol) of bis(4-(tert-butyl)phenyl)amine, 137 mg (0.15 mmol) of tris(dibenzylideneacetone)dipalladium(0), 174 mg (0.60 mmol) of tri-tert-butylphosphonium tetrafluoroborate, and 1.80 g (18.7 mmol) of sodium tert-butoxide were suspended in 60 ml of o-xylene. The suspension was three times evacuated and backfilled with argon and heated at 123° C. during 90 minutes. The dark suspension was filtered through a 3 cm layer of silica gel followed by rinsing the silica gel layer with 100 ml of toluene. The collected toluene fraction was dried over sodium sulfate and concentrated under vacuum. The crude product was dissolved in 50 ml of dichloromethane followed by addition of 100 ml of ethanol. The solution was concentrated until as suspension formed. The suspension was filtered and the solid rinsed with ethanol, giving Intermediate 9-7 as a white solid (yield: 5.24 g (83%)).

¹H-NMR (400 MHz, CD₂Cl₂): δ=7.79-7.75 (m, 1H), 7.44-7.32 (m, 5H), 7.32-7.24 (m, 6H), 7.20-7.14 (m, 2H), 7.10 (dd, 1H), 7.06 (dd, 1H), 7.00 (d, 1H), 6.85-6.78 (m, 4H), 2.27 (s, 3H), 1.43 (s, 9H), 1.39 (s, 9H), 1.37 (s, 9H), 1.34 (s, 18H).

### Compound 9

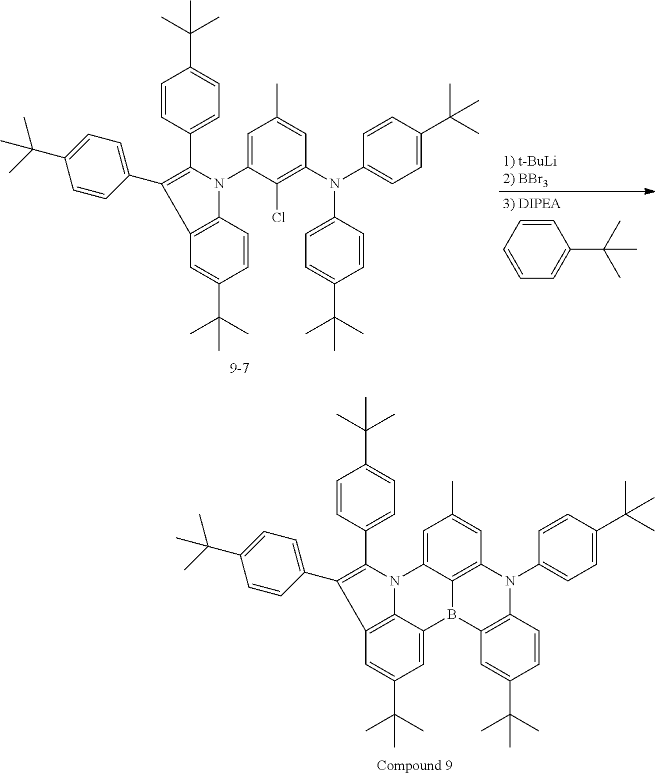

5.00 g (5.94 mmol) of Intermediate 9-7 were dissolved in 73 ml of water-free tert-butyl benzene. 6.3 ml of tert-butyl lithium (1.9 M in pentane) were slowly added at −6° C. The solution was heated up to 70° C. and pentane distilled off. Heating was continued at 65° C. during 20 minutes. The slightly grey suspension was cooled down to −52° C. and 1.15 ml (11.9 mmol mmol) of tribromoborane were slowly added. The reaction mixture was stirred at room temperature during 15 minutes and cooled down to 0° C. 2.1 ml (11.9 mmol) of N,N-diiso-propylethylamine were added and the reaction mixture heated at 165° C. during 45 hours. The dark solution was treated with 50 ml of 10% aqueous sodium acetate solution and extracted with 100 ml of toluene. The organic layer was separated, washed with water (3×50 ml), then dried over sodium sulfate, and concentrated under vacuum. The dark resin was dissolved in 50 ml of dichloromethane and 80 ml of acetonitrile. The solution was concentrated until a suspension was formed. The suspension was filtered and the solid washed with acetonitrile. The product was further purified by MPLC with the CombiFlash Companion (silica gel, heptane/0-20% gradient of ethyl acetate), followed by a second MPLC purification using the CombiFlash Companion (silica gel, cyclohexane/0-5% gradient of ethyl acetate). The solid was dissolved in 30 ml of heptane and stirred during three minutes. The resulting suspension was heated to reflux temperature and further stirred at room temperature during 1 hour. The suspension was filtered and the solid washed with heptane. The solid was purified by MPLC with the CombiFlash Companion (silica gel, heptane/0-5% toluene), giving Compound 9 as a yellow solid (yield: 405 mg (8%)).

¹H-NMR (400 MHz, CD₂Cl₂): δ=9.13 (s, 1H), 8.97 (d, 1H), 8.06 (br. s, 1H), 7.75 (d, 2H), 7.64-7.51 (m, 3H), 7.51-7.39 (m, 6H), 7.29 (d, 2H), 6.73 (br. s, 1H), 6.33-6.05 (br. s, 2H), 1.94 (br. s, 3H), 1.62 (s, 9H), 1.53 (s, 9H), 1.51 (s, 9H), 1.44 (s, 9H), 1.40 (s, 9H).

### Compound 13

### Intermediate 13-1

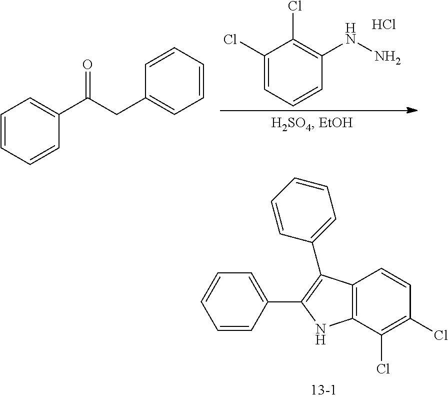

10.0 g (51.0 mmol) of 1,2-diphenylethan-1-one, and 11.4 g (53.5 mmol) of (2,3-dichlorophenyl)hydrazine hydrochloride in 100 ml of ethanol were treated with 10.0 g (102 mmol) of concentrated sulfuric acid. The yellowish suspension was heated at 82° C. during 5 hours. The resulting yellow-brown solution was poured into water and extracted with ethyl acetate. The organic phase was dried over sodium sulfate and concentrated under vacuum. The resulting solid was dissolved in hot ethanol and cooled down. The suspension was filtered and the solid rinsed with cold ethanol. The filtrate was concentrated under vacuum and further purified by MPLC with the CombiFlash Companion (silica gel, heptane). The solid was recrystallized from 50 ml heptane, giving Intermediate 13-1 (yield: 5.4 g (31%)).

¹H-NMR (400 MHz, CD₂Cl₂): δ=8.62 (s, 1H), 7.55-7.47 (m, 3H), 7.46-7.33 (m, 8H), 7.26 (d, 1H).

### Intermediate 13-2

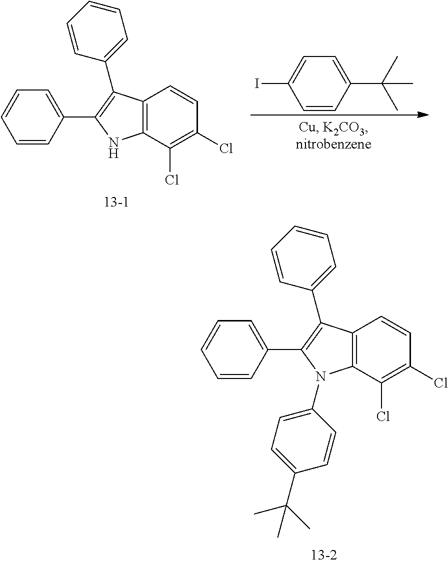 [SMILES: Clc1ccc2c(-c3ccccc3)c(-c3ccccc3)[nH]c2c1Cl ; CC(C)(C)c1ccc(I)cc1 ; CC(C)(C)c1ccc(-n2c(-c3ccccc3)c(-c3ccccc3)c3ccc(Cl)c(Cl)c32)cc1]

5.00 g (14.8 mmol) of Intermediate 13-1, 4.61 g (17.7 mmol) of 1-(tert-butyl)-4-iodobenzene, 0.94 g (14.8 mmol) of copper and 10.2 g of potassium carbonate were suspended in 50 ml of nitrobenzene. The brown suspension was heated to 208° C. during 18 hours, and cooled down to room temperature. 50 ml of toluene were added followed by filtration through a layer of celite and washing of the celite layer with 50 ml of toluene. The collected eluents were evaporated under vacuum and the resulting dark resin diluted with 20 ml of dichloromethane and 50 ml of ethanol. The resulting suspension was stirred during 1 hour, then filtered and washed with 100 ml of ethanol. The grey solid was diluted with 20 ml of dichloromethane and 50 ml of ethanol and the suspension stirred during 1 hour, followed by filtration. The solid was washed with 100 ml of ethanol giving Intermediate 13-2 as a grey solid (yield: 4.80 g (69%)).

¹H-NMR (400 MHz, CD₂Cl₂): δ=7.64 (d, 1H), 7.40-7.12 (m, 15H), 1.35 (s, 9H).

### Intermediate 13-3

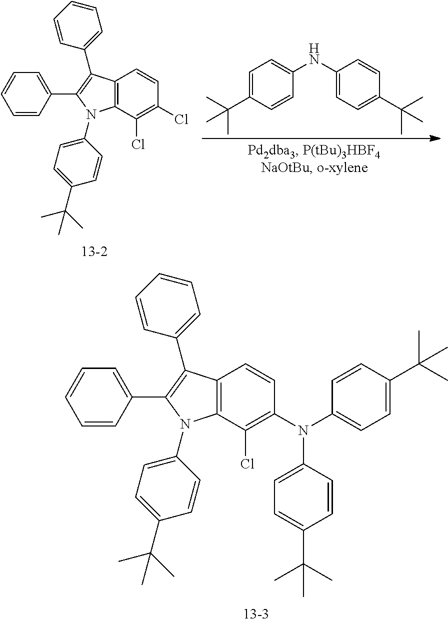

4.80 g (10.2 mmol) of Intermediate 13-2, 3.02 g (10.7 mmol) of bis(4-(tert-butyl)phenyl)amine, 187 mg (0.20 mmol) of tris(dibenzylideneacetone)dipalladium(0), 237 mg (0.81 mmol) of tri-tert-butylphosphonium tetrafluoroborate, and 2.45 g (25.5 mmol) of sodium tert-butoxide were suspended in 40 ml of o-xylene. The suspension was three times evacuated and backfilled with argon and heated at 123° C. during 20 hours. The dark suspension was filtered through a 3 cm layer of silica gel followed by rinsing the silica gel layer with 100 ml of toluene. After 10 minutes a solid precipitated from the toluene eluents. The suspension was treated with 100 ml of ethanol. The suspension was filtered, and the solid washed with ethanol, giving Intermediate 13-3 as a white solid (yield: 2.73 g (37%)).

¹H-NMR (400 MHz, CD₂Cl₂): δ=7.72 (d, 1H), 7.40-7.12 (m, 19H), 6.96-6.90 (m, 4H), 1.33 (s, 18H), 1.31 (s, 9H).

### Compound 13

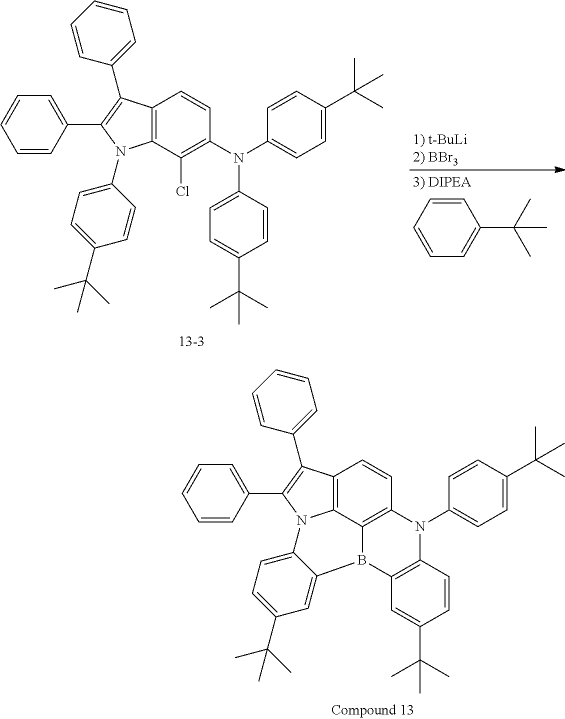

2.60 g (7.27 mmol) of Intermediate 13-3 were dissolved in 45 ml of water-free tert-butyl benzene. 3.83 ml of tert-butyl lithium (1.9 M in pentane) were slowly added at −6° C. The solution was heated up to 70° C. and pentane distilled off. Heating was continued up to 82° C. during 90 minutes. The light brown solution was cooled down to −51° C. and 0.7 ml (7.3 mmol) of tribromoborane were slowly added. The reaction mixture was stirred at room temperature during 15 minutes and cooled down to 0° C. 1.3 ml (7.3 mmol) of N,N-diisopropylethylamine were added and the reaction mixture heated up to 147° C. during 5 hours. The orange solution was treated with 50 ml of 10% aqueous sodium acetate solution and extracted with 100 ml of toluene. The organic layer was separated, washed with water (three times with 50 ml), then dried over sodium sulfate, and concentrated under vacuum. The yellow solid was suspended in 30 ml of heptane, then filtered and washed with heptane. The solid was diluted with 50 ml of dichloromethane and 50 ml of heptane were added. The solution was concentrated under vacuum until a suspension formed. The suspension was filtered and the solid washed with heptane. The solid was purified by MPLC with the CombiFlash Companion (silica gel, heptane/50% dichloromethane), giving Compound 13 as a yellow solid (yield: 485 mg (19%)).

¹H-NMR (400 MHz, CD₂Cl₂): δ=9.37 (s, 1H), 9.32 (d, 1H), 8.05 (d, 1H), 7.87-7.78 (m, 2H), 7.73 (dd, 1H), 7.66-7.51 (m, 5H), 7.47 (d, 2H), 7.45-7.23 (3 m, 7H), 7.17 (d, 1H), 6.85 (d, 1H), 1.58 (s, 9H), 1.54 (s, 18H).

### Compound 14

### Intermediate 14-1

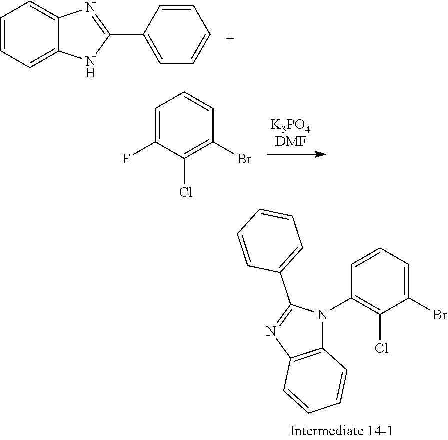

3.0 g (15.45 mmol) 2-phenyl-1H-benzo[d]imidazole and 6.47 g (30.9 mmol) 1-bromo-2-chloro-3-fluorobenzene were dissolved in 50 ml of DMF. Then 8.20 g (38.6 mmol) potassium phosphate tribasic were added. The reaction mixture was heated to 160° C. for 12 hours, then cooled to room temperature, filtrated and the solvent was removed on the rotavap. The crude product was purified by column chromatography (120 g silica, heptane/ethylacetate=6/1) to yield 4.1 g (69%) of Intermediate 14-1.

¹H NMR (400 MHz, DMSO-d₆) δ 8.03 (dd, J=8.1, 1.4 Hz, 1H), 7.84-7.79 (m, 2H), 7.55 (ddd, J=8.0, 5.0, 3.2 Hz, 3H), 7.46-7.26 (m, 5H), 7.06 (d, J=7.9 Hz, 1H).

### Intermediate 14-2

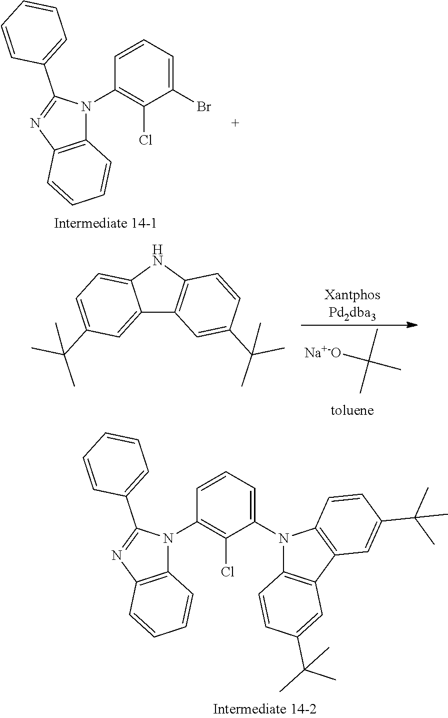

8.0 g (20.85 mmol) of Intermediate 14-1, 6.75 g (22.94 mmol) 3,6-di-tert-butyl-9H-carbazole and 5.01 g (52.1 mmol) sodium 2-methylpropan-2-olate were suspended in 240 ml of toluene. The mixture was evacuated and backfilled with argon 4 times, then argon was bubbled through for 15 minutes. 0.746 g (1.25 mmol) xantphos and 0.764 g (0.626 mmol) Pd₂(dba)₃ were added and the reaction mixture was heated to 130° C. and refluxed for 4 days. After cooling to room temperature, 100 ml of water were added, the reaction mixture was stirred for 5 minutes and then transferred to a separation funnel. The phases were separated. The water phase was extracted twice with toluene and twice with ethylacetate, the combined organic phases were washed with water and brine, dried with MgSO₄, filtered and the solvent was evaporated on the rotavap. The crude product was purified by column chromatography (heptane/dichloromethane with a gradient from 0:100 to 100:0) to yield 8.7 g (70%) of Intermediate 14-2.

1H NMR (300 MHz, CD₂Cl₂) δ 8.13 (ddd, J=5.5, 1.9, 0.6 Hz, 2H), 7.89-7.83 (m, 1H), 7.76-7.64 (m, 5H), 7.58-7.42 (m, 4H), 7.42-7.24 (m, 4H), 7.15 (dd, J=8.6, 0.7 Hz, 1H), 6.46 (dd, J=8.6, 0.7 Hz, 1H), 1.45 (d, J=1.5 Hz, 18H).

### Intermediate 14-3

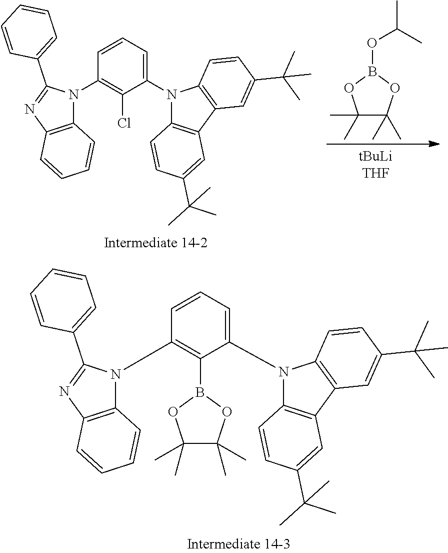

3.60 g (6.18 mmol) of Intermediate 14-2 were dissolved in 60 ml of THE and cooled to −78° C. 6.51 ml (12.37 mmol) tBuLi in pentane (1.9M) were added drop by drop (−78° C. to −65° C.). The reaction mixture was stirred at −78° C. for 45 minutes, then 5.05 ml (24.73 mmol) 2-isopropoxy-4,4,5,5-tetramethyl-1,3,2-dioxaborolane were added drop by drop at −78° C. The reaction mixture was warmed to room temperature and stirred for 24 hours. Then 10% NH₄Cl sol. was added and the reaction mixture was extracted twice with ethylacetate to give 5.5 g (99%) of Intermediate 14-3 as a yellow solid.

1H NMR (300 MHz, CD₂Cl₂) δ 8.27-8.03 (m, 2H), 7.83-7.76 (m, 3H), 7.76-7.70 (m, 1H), 7.67 (dd, J=8.0, 1.4 Hz, 1H), 7.62-7.51 (m, 1H), 7.44 (qd, J=4.3, 1.8 Hz, 3H), 7.37-7.32 (m, 2H), 7.31-7.24 (m, 2H), 7.21 (dd, J=7.1, 1.3 Hz, 1H), 7.18-7.11 (m, 1H), 6.94 (dd, J=8.6, 0.6 Hz, 1H), 1.48 (s, 6H), 1.43 (s, 6H), 1.20 (s, 18H).

### Compound 14

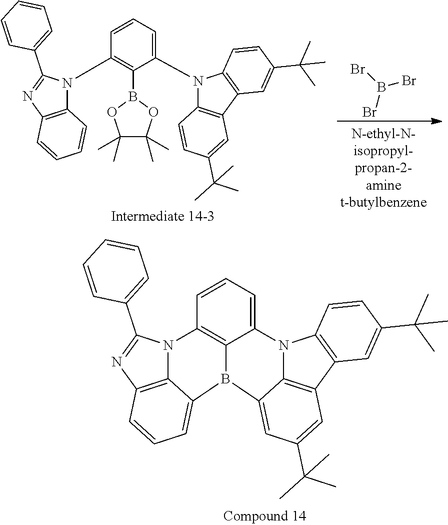

1.350 g (1.50 mmol) of Intermediate 14-3 were dissolved in 12.7 ml of t-butylbenzene and cooled to 0° C. 6.01 ml (6.01 mmol) of tribromoborane in heptane (1M) were added, then 1.050 ml (6.01 mmol)N-ethyl-N-isopropylpropan-2-amine were added within 2 minutes. The yellow solution was heated to 150° C. for 14 hours. Then it was cooled to room temperature and poured into water (ice cooling), diluted with dichlorometane and transferred to a separation funnel. The phases were separated. The water phase was extracted twice with dichloromethane, the combined organic phases were washed with water and brine, dried with MgSO₄, filtered and the solvent was evaporated on the rotavap. The crude product was purified by column chromatography (heptane/ethylacetate with a gradient from 0:100 to 100:0). Pure fractions were combined and the solvent was evaporated on the rotavap. The residue was taken in heptane and the yellow precipitate was filtered off and dried to give 115 mg (HPLC: 99%) of Compound 14.

1H NMR (300 MHz, CD₂Cl₂) δ 9.06 (d, J=1.9 Hz, 1H), 8.83 (dd, J=7.4, 1.0 Hz, 1H), 8.57 (d, J=1.9 Hz, 1H), 8.37 (d, J=8.2 Hz, 2H), 8.34-8.30 (m, 1H), 8.11 (dd, J=7.9, 1.0 Hz, 1H), 7.85-7.80 (m, 2H), 7.74 (d, J=7.6 Hz, 1H), 7.71-7.59 (m, 5H), 7.25 (d, J=8.2 Hz, 1H), 1.67 (s, 9H), 1.52 (s, 9H).

### Compound 16

### Intermediate 16-1

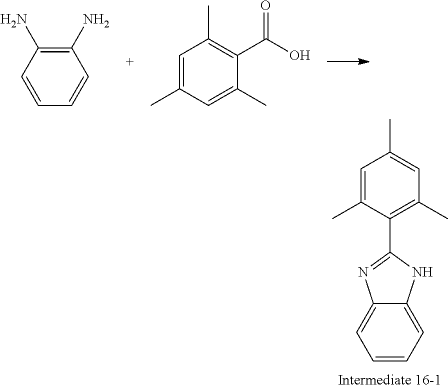

15.14 g (140.00 mmol) of benzene-1,2-diamine were mixed with 400 g (1184 mmol) of polyphosphoric acid and heated to 120° C. Then 21.95 g (134 mmol) of 2,4,6-trimethylbenzoic acid were added and the mixture was heated at 150° C. overnight (i.e. 16 h). The temperature was decreased to 60° C., the reaction mixture was added to 11 of ice water and stirred. The purple precipitate was filtered and washed with ethyl acetate. The pH of the aqueous phase was increased to 3 by adding 10N sodium hydroxide solution, the precipitate was filtered and washed with ethyl acetate. The precipitates were united to give 20.5 g (62% yield) of Intermediate 16-1.

1H NMR (300 MHz, DMSO-d6) δ 12.42 (s, 1H), 7.73-7.61 (m, 1H), 7.56-7.43 (m, 1H), 7.28-7.12 (m, 2H), 7.00 (s, 2H), 2.31 (s, 3H), 2.06 (s, 6H).

### Intermediate 16-2

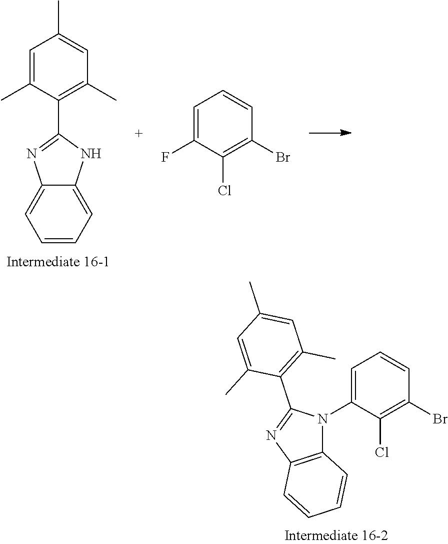

7.0 g (29.6 mmol) of Intermediate 16-1 and 12.41 g (59.2 mmol) of 1-bromo-2-chloro-3-fluorobenzene were dissolved in 300 ml of N,N-dimethylformamide. 25.20 g (118 mmol) of potassium phosphate tribasic were added. The reaction mixture was heated to 150° C. (outside temperature) for 2 hours, then cooled to room temperature and filtered. The solvent of the filtrate was removed at reduced pressure. The residue was taken in chloroform, washed several times with water, dried over magnesium sulfate, filtered and concentrated under vacuum to give 13.1 g (99% yield) of Intermediate 16-2.

1H NMR (300 MHz, Methylene Chloride-d2) δ 7.90-7.82 (m, 1H), 7.69 (dd, J=6.8, 2.8 Hz, 1H), 7.41-7.25 (m, 2H), 7.20-7.03 (m, 3H), 6.91 (s, 1H), 6.80 (d, J=1.8 Hz, 1H), 2.23 (d, J=23.7 Hz, 6H), 2.00 (s, 3H).

### Intermediate 16-3

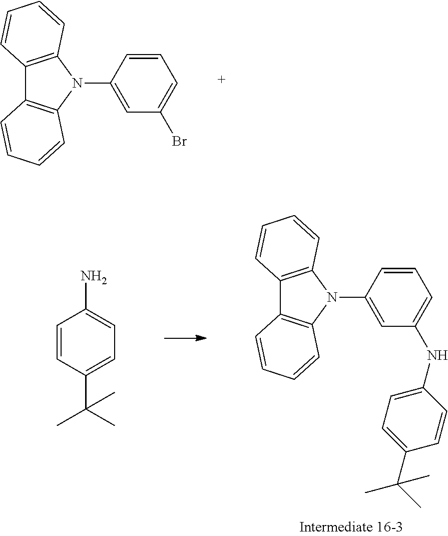 [SMILES: Brc1cccc(-n2c3ccccc3c3ccccc32)c1 ; CC(C)(C)c1ccc(N)cc1 ; CC(C)(C)c1ccc(Nc2cccc(-n3c4ccccc4c4ccccc43)c2)cc1]

25.5 g (171 mmol) of 4-(tert-butyl)aniline, 50 g (155 mmol) of 9-(3-bromophenyl)-9H-carbazole and 44.7 g (466 mmol) of sodium tert-butoxide were suspended in 300 ml of toluene. The mixture was evacuated and backfilled with argon 4 times, then 3.87 g (6.21 mmol) of 2,2′-bis(diphenylphosphino)-1,1′-binaphthyl (BINAP) and 2.84 g (3.10 mmol) of tris(dibenzylideneacetone)dipalladium(0) (Pd₂(dba)₃) were added. The reaction mixture was heated at 80° C. for 7 hours, then cooled to room temperature. 100 ml of water were added and stirred. The phases were separated, the water phase was extracted with toluene, the combined organic phases were washed with water and brine, dried over sodium sulfate, filtered and concentrated under vacuum. The crude product was recrystallized from EtOH to give 47.3 g (78% yield) of Intermediate 16-3.

¹H NMR (300 MHz, Methylene Chloride-d₂) δ 8.19 (dt, J=7.7, 1.0 Hz, 2H), 7.57-7.42 (m, 5H), 7.42-7.28 (m, 4H), 7.24 (t, J=2.1 Hz, 1H), 7.20-7.12 (m, 3H), 7.09 (ddd, J=7.7, 2.0, 0.9 Hz, 1H), 5.95 (s, 1H), 1.35 (s, 9H).

### Intermediate 16-4

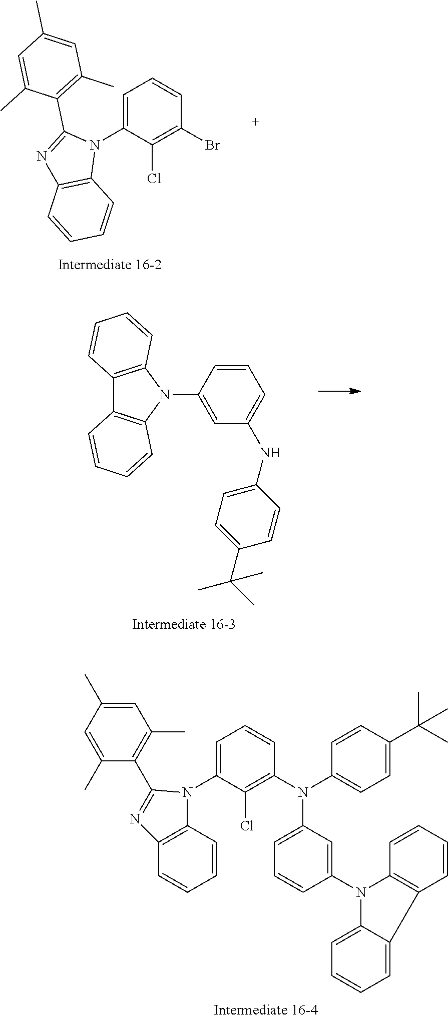 [SMILES: Cc1cc(C)c(-c2nc3ccccc3n2-c2cccc(Br)c2Cl)c(C)c1 ; CC(C)(C)c1ccc(Nc2cccc(-n3c4ccccc4c4ccccc43)c2)cc1 ; Cc1cc(C)c(-c2nc3ccccc3n2-c2cccc(N(c3ccc(C(C)(C)C)cc3)c3cccc(-n4c5ccccc5c5ccccc54)c3)c2Cl)c(C)c1]

12.4 g (24.76 mmol) of Intermediate 16-2, 9.67 g (24.76 mmol) of Intermediate 16-3 and 5.95 g (61.9 mmol) of sodium tert-butoxide were suspended in 150 ml of toluene. The mixture was evacuated and backfilled with argon 4 times. 0.859 g (1.48 mmol) Xantphos and 0.907 g (0.743 mmol) Pd₂(dba)₃ were added and the reaction mixture was heated at 110° C. for 2 hours, then cooled to room temperature.

100 ml of water were added and stirred. The phases were separated, the water phase was extracted with ethyl acetate, the combined organic phases were washed with water and brine, dried over sodium sulfate, filtered and concentrated under vacuum. The crude product was purified by column chromatography (heptane/ethyl acetate) to give 13.53 g (74% yield) of Intermediate 16-4.

¹H NMR (300 MHz, Methylene Chloride-d₂) δ 8.13 (dt, J=7.7, 1.1 Hz, 2H), 7.81 (dt, J=8.0, 1.0 Hz, 1H), 7.53-7.10 (m, 14H), 7.07-6.96 (m, 5H), 6.93 (ddd, J=8.3, 2.3, 1.0 Hz, 1H), 6.83 (s, 1H), 6.57-6.49 (m, 1H), 2.20 (s, 3H), 2.13 (s, 3H), 1.81 (s, 3H), 1.30 (s, 9H).

### Intermediate 16-5

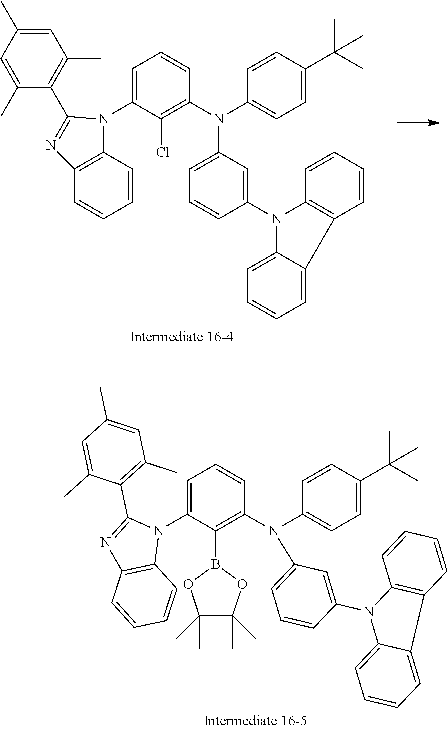

0.50 g (0.680 mmol) of Intermediate 16-4 were suspended in 25 ml of water-free tert-butylbenzene and cooled to −10° C. 0.716 ml (1.360 mmol) of tert-butyllithium (1.9M in pentane) were slowly added. The reaction mixture was warmed up to room temperature and stirred overnight. After cooling to −10° C., 0.597 ml (2.72 mmol) of 2-isopropoxy-4,4,5,5-tetramethyl-1,3,2-dioxaborolane were slowly added. The reaction mixture was warmed to room temperature and stirred for 2 hours, then 10% of aqueous ammonium chloride solution was added. The phases were separated, the water phase was extracted with ethylaceteate, the combined organic phases were washed with water and brine, dried over sodium sulfate, filtered and concentrated under vacuum to give 0.556 g (99% yield) of Intermediate 16-5 as a yellow solid.

LC-MS: 825.4 [M−H]⁻

### Compound 16

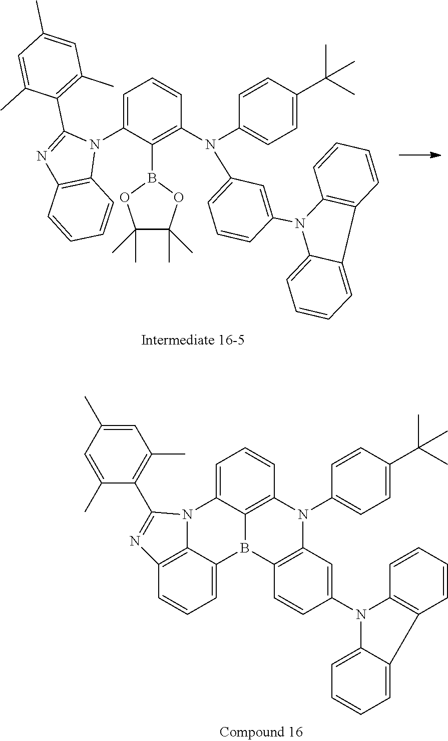 [SMILES: Cc1cc(C)c(-c2nc3ccccc3n2-c2cccc(N(c3ccc(C(C)(C)C)cc3)c3cccc(-n4c5ccccc5c5ccccc54)c3)c2B2OC(C)(C)C(C)(C)O2)c(C)c1 ; Cc1cc(C)c(-c2nc3cccc4c3n2-c2cccc3c2B4c2ccc(-n4c5ccccc5c5ccccc54)cc2N3c2ccc(C(C)(C)C)cc2)c(C)c1]

0.73 g (0.680 mmol) of Intermediate 16-5 were dissolved in 20 ml of water free tert-butylbenzene and cooled to 0° C. 2.72 ml (2.72 mmol) of tribromoborane (1 M in heptane) were added, then 0.475 ml (2.72 mmol) of N-ethyl-N-isopropylpropan-2-amine were added within 2 minutes. The dark yellow solution was heated at 150° C. for 14 hours. Then it was cooled to room temperature and poured into ice-water, diluted with ethyl acetate and the phases were separated. The water phase was extracted with ethyl acetate, the combined organic phases were washed with water and brine, dried over magnesium sulfate, filtered and concentrated under vacuum. The crude product was purified by column chromatography (heptane/ethyl acetate) to give 62 mg (13% yield) of Compound 16.

### Compound 18

### Intermediate 18-1

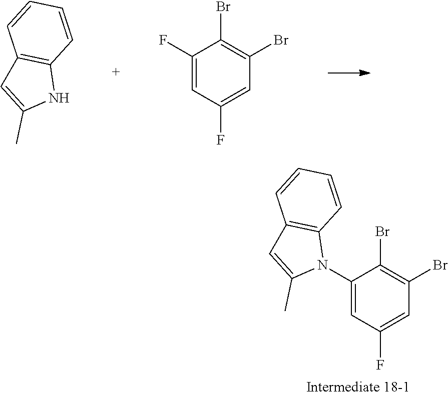

To 10.4 g (38.1 mmol) of 1,2-dibromo-3,5-difluoro benzene and 5.00 g (38.1 mmol) of 2-methyl-1H-indole in 100 ml of water free DMF, 40.5 g (191 mmol) of potassium phosphate tribasic were added under nitrogen. The reaction mixture was stirred at 50° C. for 18 h under nitrogen. The solids were filtered off and the solvent was removed in vacuum. Column chromatography on silica gel with heptane and then heptane/ethyl acetate 1/1 gave 3.88 g of the Intermediate 18-1 (27% yield).

MS (ESI) m/z=384 (M+1)

### Intermediate 18-2

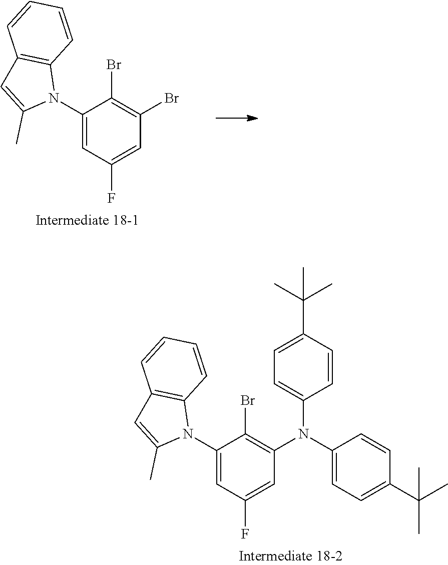 [SMILES: Cc1cc2ccccc2n1-c1cc(F)cc(Br)c1Br ; Cc1cc2ccccc2n1-c1cc(F)cc(N(c2ccc(C(C)(C)C)cc2)c2ccc(C(C)(C)C)cc2)c1Br]

To 3.88 g (10.1 mmol) of the Intermediate 18-1 and 3.14 g (11.1 mmol) of bis(tert-butyl-phenyl) amine in 50 ml of water free tert-butyl-benzene, 1.36 g (14.2 mmol) of sodium tert-butoxide were added under argon. The reaction mixture was degassed with argon. 590 mg (1.00 mmol) of 4,5-bis(diphenylphosphino)-9,9-dimethylxanthene (Xantphos) and 460 mg (0.510 mmol) of tris(dibenzylideneacetone)dipalladium(0) were added. The reaction mixture was degassed with argon. The reaction mixture was stirred at 150° C. under argon for 1 h. 200 mg of sodium cyanide in 25 ml of water were added and the reaction mixture was stirred at 100° C. for 30 min. 100 ml of water and 100 ml of toluene were added and the organic phase was separated. The organic phase was washed with water and dried with magnesium sulfate. The solvent removed in vacuum. Column chromatography on silica gel with heptane and then ethyl acetate gave 2.8 g of the Intermediate 18-2 (47% yield).

MS (ESI) m/z=583 (M+1)

### Intermediate 18-3

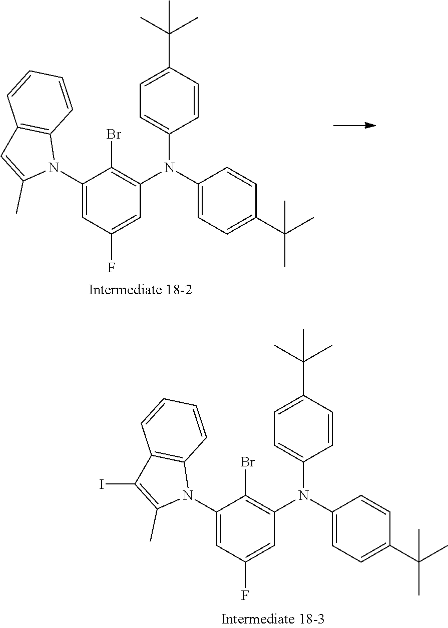 [SMILES: Cc1cc2ccccc2n1-c1cc(F)cc(N(c2ccc(C(C)(C)C)cc2)c2ccc(C(C)(C)C)cc2)c1Br ; Cc1c(I)c2ccccc2n1-c1cc(F)cc(N(c2ccc(C(C)(C)C)cc2)c2ccc(C(C)(C)C)cc2)c1Br]

To 11.0 g (18.9 mmol) of the Intermediate 18-2 and 4.47 g (56.5 mmol) of pyridine in 100 ml of chloroform, 5.26 g (20.7 mmol) of iodine were added under nitrogen. The reaction mixture was stirred for 30 min and additional 1.20 g (4.71 mmol) of iodine were added. The reaction mixture was filtered on silica gel. 300 ml of methanol and sodium metabisulfite (Na₂S₂O₅) were added. The precipitated product was filtered off. The solids were dissolved in chloroform and the solids were filtered off. The solvent was removed in vacuum. The product was crystallized from toluene/isopropanol 1/9 to give 7.2 g of the Intermediate 18-3 (50% yield).

¹H-NMR (400 MHz, CD₂Cl₂)⋅=7.44 (m, 1H), 7.35 (d, 4H), 7.24 (m, 2H), 7.17 (dd, 1H), 7.04 (dd, 1H), 6.98 (m, 5H), 2.34 (s, 3H), 1.36 (s, 18H).

### Intermediate 18-4

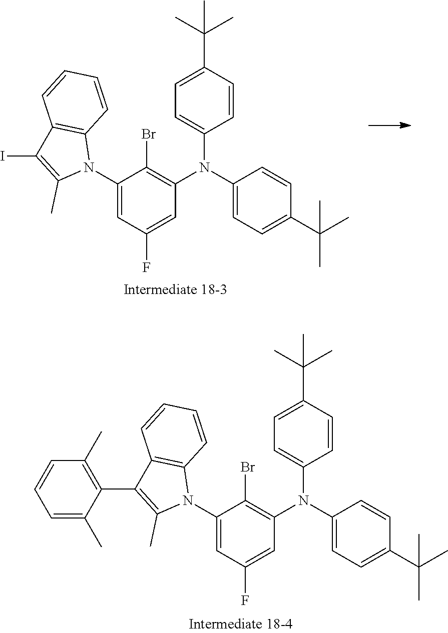 [SMILES: Cc1c(I)c2ccccc2n1-c1cc(F)cc(N(c2ccc(C(C)(C)C)cc2)c2ccc(C(C)(C)C)cc2)c1Br ; Cc1cccc(C)c1-c1c(C)n(-c2cc(F)cc(N(c3ccc(C(C)(C)C)cc3)c3ccc(C(C)(C)C)cc3)c2Br)c2ccccc12]

To 7.20 g (10.2 mmol) of the Intermediate 18-3, 654 mg (2.03 mmol) of tetrabutylammonium bromide and 3.04 g (20.3 mmol) of (2,6-dimethylphenyl)boronic acid in a mixture of 150 ml of toluene, 60 ml of ethanol and 30 ml of water, 4.21 g (30.4 mmol) of potassium phosphate were added under Argon. The reaction mixture was degassed with argon. 833 mg (2.03 mmol) of dicyclohexyl(2′,6′-dimethoxy-[1,1′-biphenyl]-2-yl)phosphine and 228 mg (1.02 mmol) of palladium (II) acetate were added and the reaction mixture was degassed with argon. The reaction mixture was stirred for 3 h at 90° C. 500 mg of sodium cyanide were added and the reaction mixture was stirred for 20 min at 90° C. The organic phase was separated and was washed with water. The organic phase was dried with magnesium sulfate and the solvent was removed in vacuum. Column chromatography on silica gel with heptane and then toluene/heptane 55/45 gave 7.56 g of the Intermediate 18-4 (25% yield).

¹H-NMR (300 MHz, CD₂Cl₂)⋅=7.34 (m, 4H), 7.15 (m, 9H), 6.98 (m, 4H), 2.14 (s, 3H), 2.06 (s, 3H), 2.01 (s, 3H), 1.35 (18H).

### Compound 18

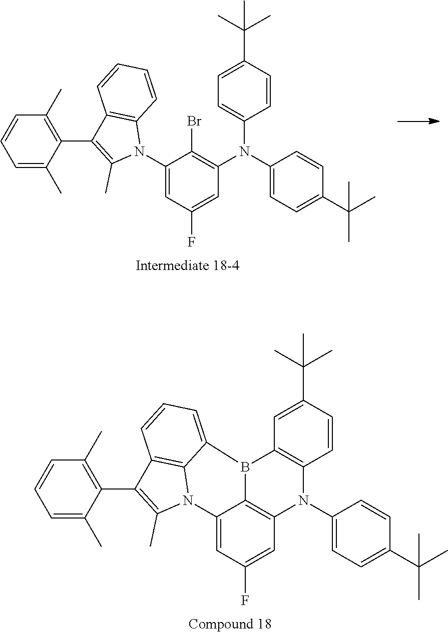 [SMILES: Cc1cccc(C)c1-c1c(C)n(-c2cc(F)cc(N(c3ccc(C(C)(C)C)cc3)c3ccc(C(C)(C)C)cc3)c2Br)c2ccccc12 ; Cc1cccc(C)c1-c1c(C)n2c3c(cccc13)B1c3cc(C(C)(C)C)ccc3N(c3ccc(C(C)(C)C)cc3)c3cc(F)cc-2c31]

To 4.00 g (5.82 mmol) of the Intermediate 18-4 in 60 ml of water free tert-butylbenzene, 6.84 ml (11.6 mmol) of tert-butyllithium 1.7 M in pentane were added at 0° C. under argon. The reaction mixture was stirred for 60 min at 60° C. under argon. The reaction mixture was cooled to 0° C. and 1.35 ml (11.6 mmol) of tribromoborane were added under argon. The reaction mixture was stirred for 10 min at 0° C. and was then warmed up to 25° C. After 10 min the reaction mixture was cooled to 0° C. and 2.03 ml (11.6 mmol) of di-isopropyl-ethylamine were added. After 15 min the reaction mixture was stirred at 150° C. for 24 h. The reaction mixture was cooled to 25° C. 20 ml of ammonium hydrogen chloride solution and ethyl acetate were added and the organic phase was separated. The organic phase was dried with sodium sulfate and the solvent was removed in vacuum. The product was purified first by column chromatography on silica gel with heptane/ethyl acetate 97/3, then with a column chromatography on silica gel with heptane and then toluene to give 260 mg of the Compound 18 (7% yield).

¹H-NMR (400 MHz, CDCl₃)⋅=9.00 (s, 1H), 8.67 (d, 1H), 7.64 (m, 2H), 7.44 (m, 4H), 7.19 (m, 5H), 6.68 (d, 1H), 6.08 (s, 1H), 3.25 (s, 3H), 2.05 (s, 6H), 1.41 (s, 18H).

MS (ESI) m/z=617 (M+1)

### Compound 19

### Intermediate 19-1

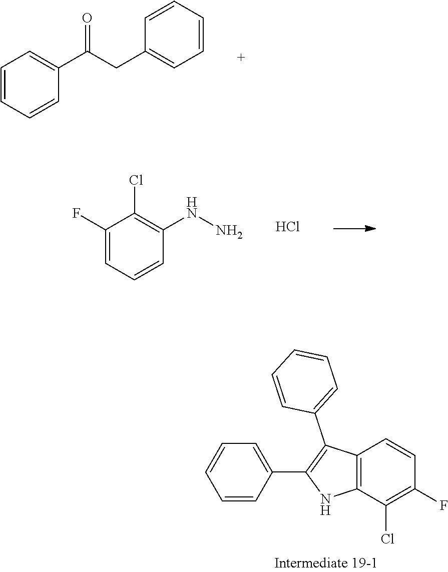 [SMILES: O=C(Cc1ccccc1)c1ccccc1 ; NNc1cccc(F)c1Cl ; Cl ; Fc1ccc2c(-c3ccccc3)c(-c3ccccc3)[nH]c2c1Cl]

22.6 g (115 mmol) of 1,2-diphenylethan-1-one and 23.8 g (121 mmol) of (2-chloro-3-fluorophenyl)hydrazine hydrochloride in 222 mL of ethanol were treated with 22.6 g (230 mmol) of concentrated sulfuric acid. The suspension was heated at 100° C. during 8 h. The resulting dark solution was poured into water and extracted with ethyl acetate. The organic phase was dried over sodium sulfate and concentrated under vacuum. The crude product was purified via combi-flash chromatography eluting with a mixed solvent of ethyl acetate and heptane to give a brown oil, which was further purified by reverse combi-flash chromatography eluting with a mixed solvent of MeOH and H₂O to give 15.4 g (40% yield) of Intermediate 19-1 as a yellow solid.

LC-MS: 320.0 [M−H]⁻

### Intermediate 19-2

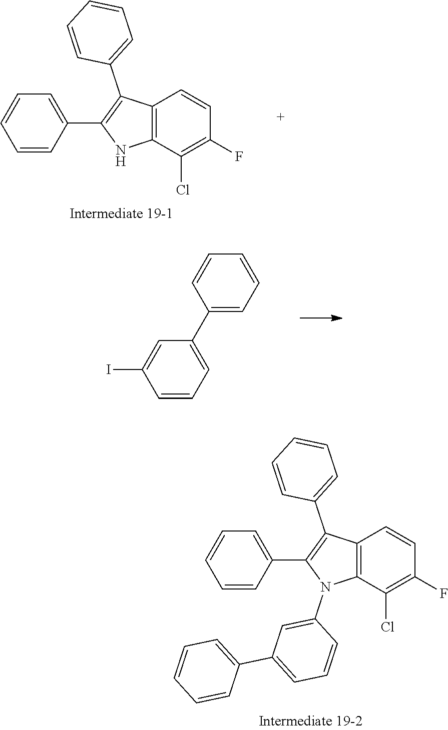 [SMILES: Fc1ccc2c(-c3ccccc3)c(-c3ccccc3)[nH]c2c1Cl ; Ic1cccc(-c2ccccc2)c1 ; Fc1ccc2c(-c3ccccc3)c(-c3ccccc3)n(-c3cccc(-c4ccccc4)c3)c2c1Cl]

13.3 g (40 mmol) of Intermediate 19-1, 17.5 g (60 mmol) of 3-iodo-1,1′-biphenyl, 2.5 g (40 mmol) of copper and 27.6 g (200 mmol) of potassium carbonate were suspended in 160 mL of nitrobenzene. The brown suspension was heated to 215° C. during 66 h, then cooled down to room temperature. 50 mL of toluene were added followed by filtration through a layer of celite and washing of the celite layer with toluene. The collected eluents were evaporated under vacuum and the crude black product was purified via combi-flash chromatography eluting with a mixed solvent of toluene and heptane. The resulting solid was further purified by successive recrystallizations from MeOH and 1-methoxy-2-propanol to give 13.05 g (69% yield) of Intermediate 19-2 as a yellow solid.

LC-MS: 474.2 [M+H]⁺

### Intermediate 19-3

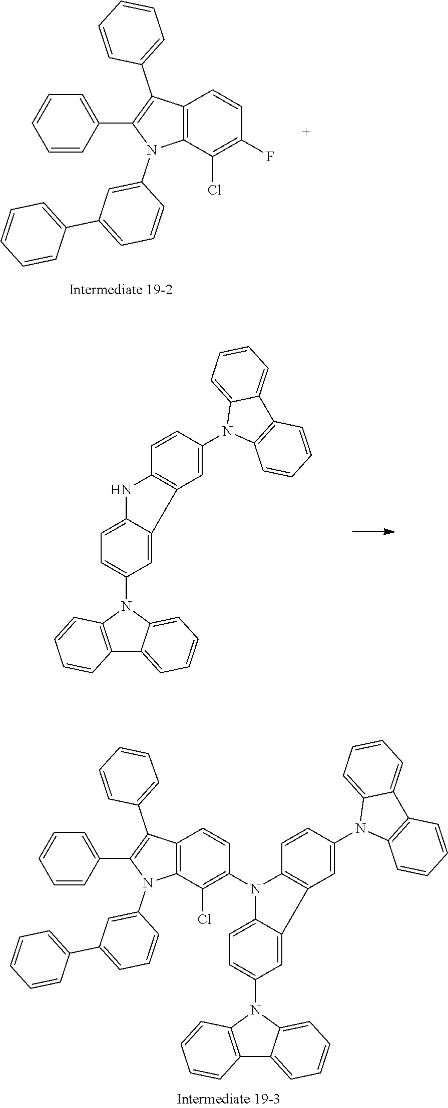 [SMILES: Fc1ccc2c(-c3ccccc3)c(-c3ccccc3)n(-c3cccc(-c4ccccc4)c3)c2c1Cl ; c1ccc2c(c1)c1ccccc1n2-c1ccc2[nH]c3ccc(-n4c5ccccc5c5ccccc54)cc3c2c1 ; Clc1c(-n2c3ccc(-n4c5ccccc5c5ccccc54)cc3c3cc(-n4c5ccccc5c5ccccc54)ccc32)ccc2c(-c3ccccc3)c(-c3ccccc3)n(-c3cccc(-c4ccccc4)c3)c12]

4.0 g (8.45 mmol) of Intermediate 19-2, 4.20 g (10.7 mmol) of 9′H-9,3′:6′,9″-tercarbazole (prepared according to Albrecht, K. et al, Angew. Chem. nt. Ed. 2015, 54, 5677) and 5.8 g (42.3 mmol) of potassium carbonate were suspended in 210 mL of DMSO. The suspension was stirred at 150° C. during 47 h. Then the reaction was cooled to room temperature and the DMSO was removed by distillation. The resulting solid was washed with MeOH, then purified via combi-flash chromatography eluting a mixed solvent of toluene and heptane to give 2.55 g (30% yield) of Intermediate 19-3 as an off-white solid.

LC-MS: 951.6 [M+H]⁺

### Compound 19

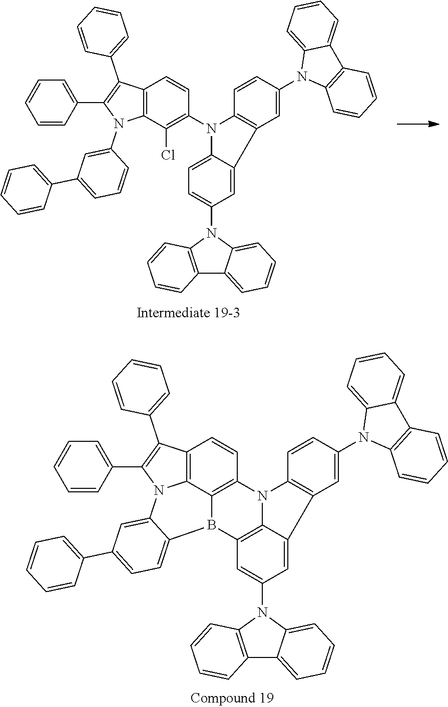 [SMILES: Clc1c(-n2c3ccc(-n4c5ccccc5c5ccccc54)cc3c3cc(-n4c5ccccc5c5ccccc54)ccc32)ccc2c(-c3ccccc3)c(-c3ccccc3)n(-c3cccc(-c4ccccc4)c3)c12 ; c1ccc(-c2ccc3c(c2)-n2c(-c4ccccc4)c(-c4ccccc4)c4ccc5c(c42)B3c2cc(-n3c4ccccc4c4ccccc43)cc3c4cc(-n6c7ccccc7c7ccccc76)ccc4n-5c23)cc1]

2.52 g (2.65 mmol) of Intermediate 19-3 were dissolved in 32 mL of water-free tert-butyl benzene. 4.20 mL of tert-butyl lithium (1.9 M in pentane) were slowly added at −10° C. The solution was heated up to 70° C. and pentane distilled off. Heating was continued up to 80° C. during 2 h. The resulting solution was cooled down to −55° C. and 0.5 mL (5.3 mmol) of tribromoborane were slowly added. The reaction mixture was allowed to reach room temperature by removing the cooling bath, then cooled down to 0° C. 0.93 mL (5.3 mmol) of N,N-diisopropylethylamine were added and the reaction mixture heated up to 160° C. during 20 hours. After cooling to room temperature, the reaction mixture was diluted with toluene and treated with 50 mL of 10% aqueous sodium acetate solution. The organic layer was separated, washed twice with water, then dried over magnesium sulfate, and concentrated under vacuum. The crude product was purified via combi-flash chromatography (silica gel, 20-60% toluene/heptane), and the resulting solid was further purified by recrystallization from ethyl acetate to give 137 mg (6% yield) of Compound 19 as a yellow solid.

LC-MS: 925.5 [M+H]⁺

### Compound 20

### Intermediate 20-1

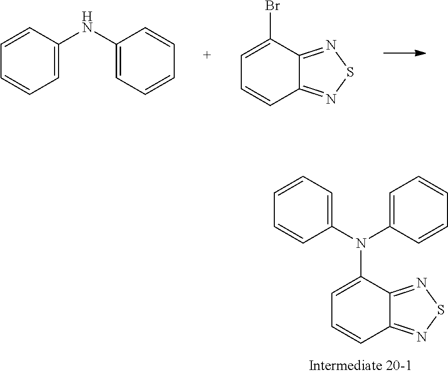 [SMILES: c1ccc(Nc2ccccc2)cc1 ; Brc1cccc2nsnc12 ; c1ccc(N(c2ccccc2)c2cccc3nsnc23)cc1]

6.42 g (38.0 mmol) of diphenylamine and 8.57 g (39.8 mmol) of 4-bromobenzo[c][1,2,5]thiadiazole were suspended in 127 mL of toluene. The mixture was evacuated and backfilled with argon 3 times. 0.881 g (3.03 mmol) of tri-tert-butylphosphonium tetrafluoroborate and 0.695 g (0.759 mmol) of Pd₂(dba)₃ were added and the reaction mixture was heated to 100° C. and refluxed for 2.5 h, then cooled to room temperature. The reaction mixture was filtered off and filtrated over silica gel to give 11.4 g (99.1%) of intermediate 20-1 as an orange solid.

LC-MS: 304.0 [M+H]⁺

### Intermediate 20-2

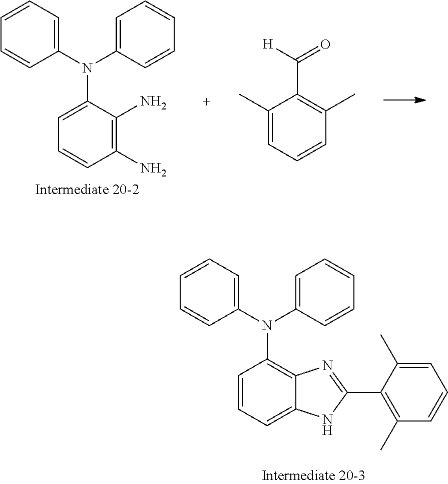

9.00 g (29.7 mmol) of Intermediate 20-1 were dissolved in 100 mL of THE and diluted with 222 mL of EtOH. 247 mg (1.04 mmol) of cobalt(II) chloride hexahydrate were added and then 22.45 g (593 mmol) of sodium borohydride. The reaction mixture was heated to 150° C. for 2 h, then cooled to room temperature and the reaction mixture was filtered through a pad of Celite and washed with TH F. The filtrate was concentrated. The residue was taken in tert-butyl methylether, the solution was washed several times with water, dried over magnesium sulfate. After the solvent was removed under reduced pressure, the crude product was purified by column chromatography eluting with a mixed solvent of heptane and ethyl acetate to give 4.99 g (61% yield) of intermediate 20-2 as a brown solid.

LC-MS: 276.0 [M+H]⁺

### Intermediate 20-3

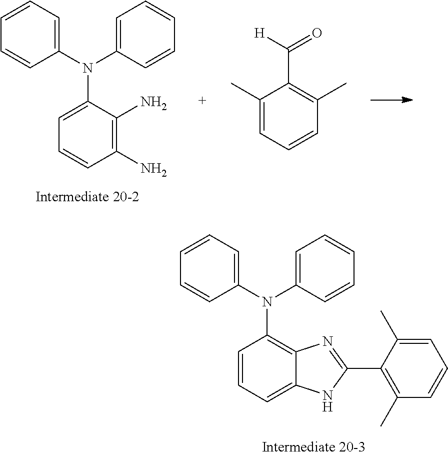

4.99 g (18.1 mmol) of intermediate 20-2 and 2.26 g (21.7 mmol) of sodium hydrogen sulfite were suspended in 25 mL of dimethyl acetamide. Then 2.55 g (19.0 mmol) of 2,6-dimethylbenzaldehyde dissolved in 11 mL of dimethyl acetamide were added via a syringe, and the mixture was stirred at 100° C. for 16 h. After the reaction mixture was cooled at room temperature, it was poured into 110 mL of water. The solid was filtered off, washed with water and dried in vacuum at 70° C. to give 6.90 g (97.7% yield) of Intermediate 20-3 as a beige solid. This product was used for the next reaction without purification.

LC-MS: 390.3 [M+H]⁺

### Intermediate 20-4

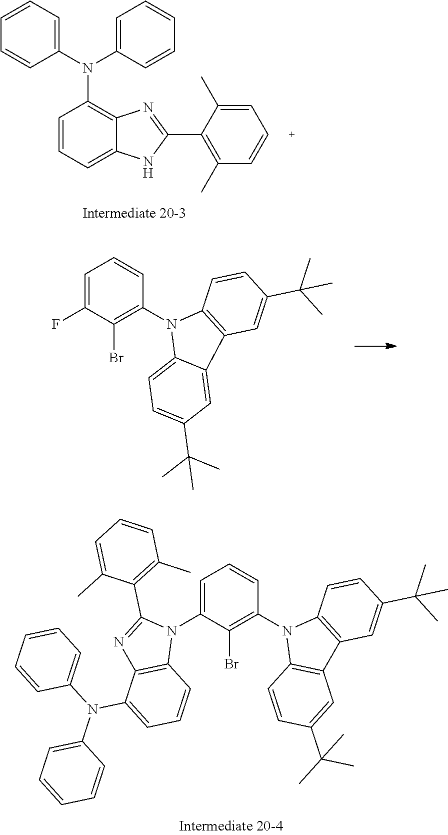 [SMILES: Cc1cccc(C)c1-c1nc2c(N(c3ccccc3)c3ccccc3)cccc2[nH]1 ; CC(C)(C)c1ccc2c(c1)c1cc(C(C)(C)C)ccc1n2-c1cccc(F)c1Br ; Cc1cccc(C)c1-c1nc2c(N(c3ccccc3)c3ccccc3)cccc2n1-c1cccc(-n2c3ccc(C(C)(C)C)cc3c3cc(C(C)(C)C)ccc32)c1Br]

1.97 g (5.06 mmol) of Intermediate 20-3, 2.29 g (5.06 mmol) of 9-(2-bromo-3-fluorophenyl)-3,6-di-tert-butyl-9H-carbazole and 4.29 g (20.2 mmol) of potassium phosphate were suspended in 17 mL of dimethyl formamide. The mixture was stirred at 140° C. for 14.5 h. the reaction mixture was diluted with 51 mL of water. The solid was filtered off, washed with water and dried in vacuum at 70° C. The crude product was purified by column chromatography eluting with a mixed solvent of ethylacetate and toluene to give 3.35 g (81% yield) of Intermediate 20-4 as a beige solid.

LC-MS: 821 [M+H]⁺

### Intermediate 20-5

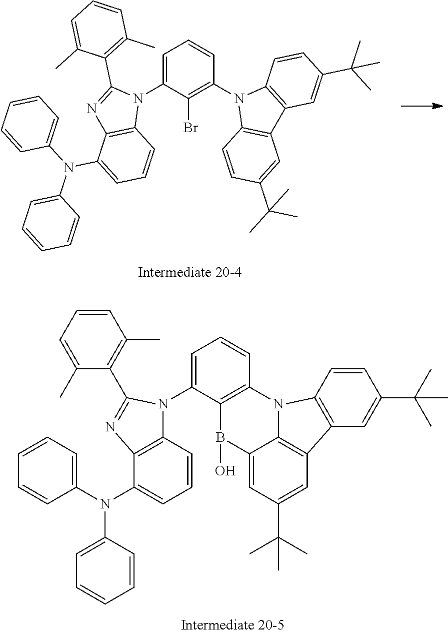 [SMILES: Cc1cccc(C)c1-c1nc2c(N(c3ccccc3)c3ccccc3)cccc2n1-c1cccc(-n2c3ccc(C(C)(C)C)cc3c3cc(C(C)(C)C)ccc32)c1Br ; Cc1cccc(C)c1-c1nc2c(N(c3ccccc3)c3ccccc3)cccc2n1-c1cccc2c1B(O)c1cc(C(C)(C)C)cc3c4cc(C(C)(C)C)ccc4n-2c13]

1.00 g (1.22 mmol) of Intermediate 20-4 were dissolved in 10 mL of tert-butylbenzene and the solution was cooled at −5° C. 1.28 mL (2.43 mmol) of tert-butyl lithium were added dropwise to the solution at −5° C. After 10 min, the reaction mixture was cooled at −78° C., and then 0.24 mL (2.50 mmol) of boron tribromide were added. After 10 min, the reaction mixture warmed to −10° C., and 1.09 mL (6.27 mmol) of N,N-diisopropyethylamine were added. The reaction mixture was stirred at 145° C. for 2.5 h. The reaction mixture was cooled at room temperature and diluted with toluene and potassium acetate aqueous solution. The aqueous layer was extracted with toluene and the organic layer was washed with brine and dried over sodium sulphate. After removal of the solvent at the reduced pressure, the crude product was purified by column chromatography eluting with a mixed solvent of dichloromethane and toluene to give 303 mg (32% yield) of Intermediate 20-5 as a brown resin.

LC-MS: 769.4 [M+H]⁺

### Compound 20

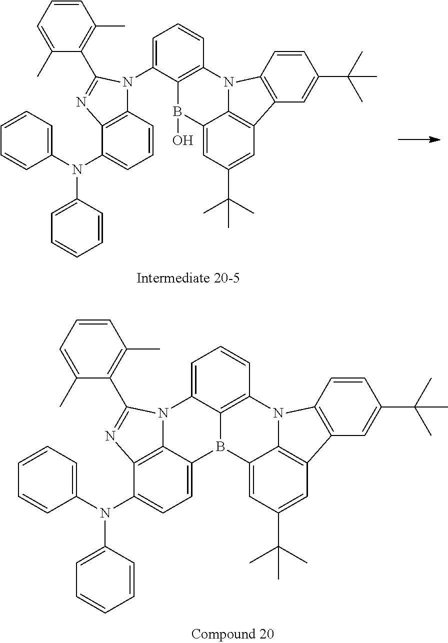 [SMILES: Cc1cccc(C)c1-c1nc2c(N(c3ccccc3)c3ccccc3)cccc2n1-c1cccc2c1B(O)c1cc(C(C)(C)C)cc3c4cc(C(C)(C)C)ccc4n-2c13 ; Cc1cccc(C)c1-c1nc2c(N(c3ccccc3)c3ccccc3)ccc3c2n1-c1cccc2c1B3c1cc(C(C)(C)C)cc3c4cc(C(C)(C)C)ccc4n-2c13]

244 mg (0.32 mmol) of Intermediate 20-5 were dissolved in 2 mL of 1,2-dichlorobenzene, and the solution was cooled at −5° C. Then 0.95 mL (0.24 mmol) of boron tribromide and 0.17 mL (0.12 mmol) of N,N-diisopropylethylamine were added, and the mixture was stirred at 145° C. for 1 h. The reaction mixture was cooled at room temperature and the solvent was removed at the reduced pressure to give the Compound 20.

LC-MS: 795 [M+H]⁺

### Compound 21

### Intermediate 21-1

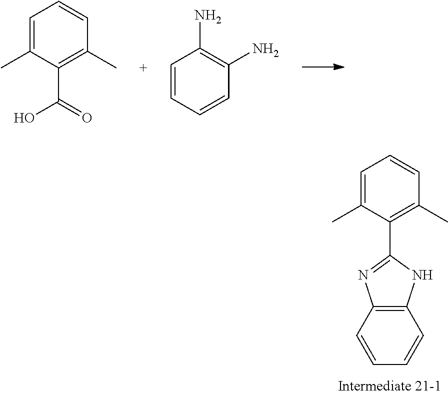

5.68 g (52.5 mmol) of benzene-1,2-diamine were mixed with 152 g (450 mmol) of polyphosphoric acid and heated to 120° C. Then 7.51 g (50 mmol) of 2,6-dimethylbenzoic acid were added and the mixture was heated at 150° C. for 8 hours. The temperature was decreased to 30° C., the reaction mixture was added to 200 ml of ice water and stirred. The pH of the aqueous phase was increased to 5 by adding 160 ml of a 30% sodium hydroxide solution. The precipitate was filtered, washed with water and ethyl acetate and dried to give 10 g (90% yield) of Intermediate 21-1.

¹H NMR (400 MHz, DMSO-d₆) δ 12.54 (s, 1H), 7.66 (t, J=6.8 Hz, 2H), 7.51 (d, J=7.5 Hz, 1H), 7.18 (q, J=8.2, 7.4 Hz, 4H), 2.59 (s, 3H), 2.34 (s, 3H).

### Intermediate 21-2

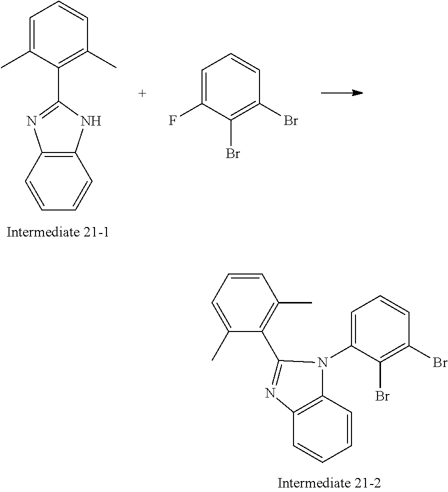

9.83 g (44.2 mmol) of Intermediate 21-1 and 22.46 g (88 mmol) of 1,2-dibromo-3-fluorobenzene were dissolved in 120 ml of N,N-dimethylformamide. 46.90 g (121 mmol) of potassium phosphate tribasic were added. The reaction mixture was heated at 150° C. (outside temperature) for 2 hours, then cooled to room temperature and filtered. The solvent of the filtrate was removed at reduced pressure. The residue was taken in ethyl acetate, washed several times with water, dried over magnesium sulfate, filtered and concentrated under vacuum. Precipitation in heptane gave 15.0 g (72% yield) of Intermediate 21-2.

1H NMR (300 MHz, Methylene Chloride-d2) δ 7.88-7.82 (m, 1H), 7.68 (dd, J=7.9, 1.7 Hz, 1H), 7.33 (dtd, J=14.4, 7.3, 1.4 Hz, 2H), 7.23-7.15 (m, 1H), 7.15-7.07 (m, 3H), 7.05 (dd, J=7.9, 1.7 Hz, 1H), 6.99-6.94 (m, 1H), 2.21 (s, 3H), 2.09 (s, 3H).

### Intermediate 21-3

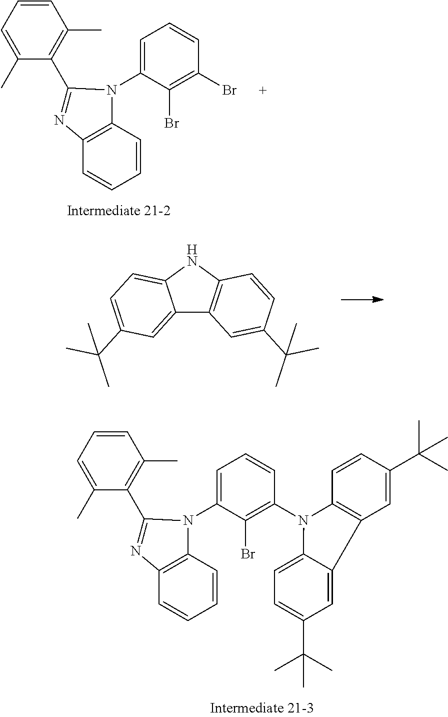 [SMILES: Cc1cccc(C)c1-c1nc2ccccc2n1-c1cccc(Br)c1Br ; CC(C)(C)c1ccc2[nH]c3ccc(C(C)(C)C)cc3c2c1 ; Cc1cccc(C)c1-c1nc2ccccc2n1-c1cccc(-n2c3ccc(C(C)(C)C)cc3c3cc(C(C)(C)C)ccc32)c1Br]

15.1 g (33.1 mmol) of Intermediate 21-2, 9.25 g (33.1 mmol) of 3,6-di-tert-butyl-9H-carbazole and 7.95 g (83 mmol) of sodium tert-butoxide were suspended in 250 ml of xylene. The mixture was evacuated and backfilled with argon 4 times. 1.173 g (1.986 mmol) of Xantphos and 0.928 g (0.993 mmol) of Pd₂(dba)₃ were added and the reaction mixture was heated at 135° C. for 27 hours. After cooling to room temperature, 350 ml of water were added and the reaction mixture was stirred. The phases were separated, the water phase was extracted with ethyl acetate, the combined organic phases were washed with water and brine, dried over magnesium sulfate, filtered and concentrated under vacuum. The crude product was purified by column chromatography (heptane/ethyl acetate) to give 13.0 g (60% yield) of Intermediate 21-3.

1H NMR (300 MHz, Chloroform-d) δ 8.17-8.12 (m, 2H), 7.98 (d, J=6.8 Hz, 1H), 7.55-7.35 (m, 6H), 7.33-7.27 (m, 3H), 7.16-7.07 (m, 3H), 6.58 (d, J=8.7 Hz, 1H), 2.28 (s, 3H), 2.23 (s, 3H), 1.48 (s, 9H), 1.47 (s, 9H).

### Compound 21

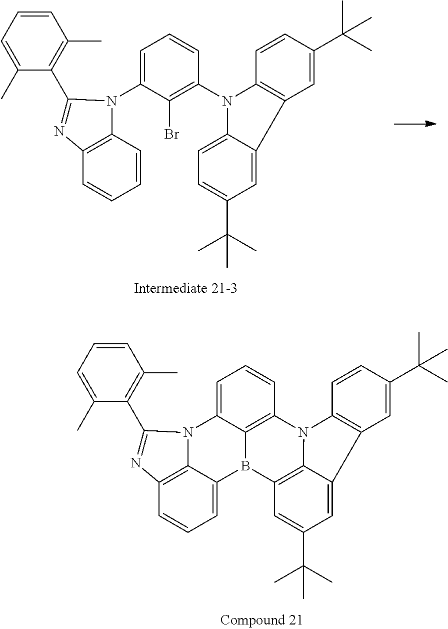 [SMILES: Cc1cccc(C)c1-c1nc2ccccc2n1-c1cccc(-n2c3ccc(C(C)(C)C)cc3c3cc(C(C)(C)C)ccc32)c1Br ; Cc1cccc(C)c1-c1nc2cccc3c2n1-c1cccc2c1B3c1cc(C(C)(C)C)cc3c4cc(C(C)(C)C)ccc4n-2c13]

4.80 g (7.33 mmol) of Intermediate 21-3 were dissolved in 130 ml of water-free tert-butylbenzene. 3.0 ml (8.10 mmol) of n-butyllithium (2.7M in pentane) were slowly added at −5° C. and then heated up to room temperature within 30 minutes. The yellowish solution was cooled down to −34° C. and 2.80 ml (29.2 mmol) of tribromoborane were added. The reaction mixture was warmed up to room temperature within 30 minutes and then cooled to 0° C. 4.12 ml (29.3 mmol) of N,N-diisopropylethylamine were added and the reaction mixture was heated up to 160° C. and stirred at this temperature for 20 hours. The yellow suspension was cooled down and treated with 30 ml of 10% aqueous sodium acetate solution and extracted with 100 ml of ethyl acetate. The organic layer was separated, washed with water, dried over sodium sulfate and concentrated under vacuum. The isolated product was precipitated in heptane and then purified by column chromatography (ethyl acetate/methanol) to give 2.67 g (62% yield) of Compound 21.

1H NMR (300 MHz, Methylene Chloride-d2) δ 9.10 (d, J=1.9 Hz, 1H), 8.87 (dd, J=7.5, 1.0 Hz, 1H), 8.57 (d, J=1.9 Hz, 1H), 8.43-8.29 (m, 3H), 8.16 (dd, J=7.8, 0.9 Hz, 1H), 7.76 (t, J=7.6 Hz, 1H), 7.67 (dd, J=9.0, 2.0 Hz, 1H), 7.61 (d, J=8.4 Hz, 1H), 7.49 (dd, J=8.2, 7.1 Hz, 1H), 7.36-7.29 (m, 2H), 7.01-6.94 (m, 1H), 2.14 (s, 6H), 1.67 (s, 9H), 1.52 (s, 9H).

### Compound 22

### Intermediate 22-1

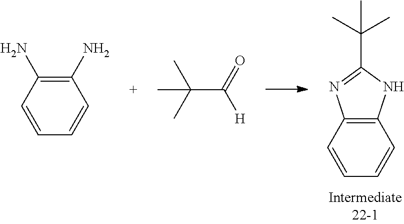

30.0 g (0.28 mol) of benzene-1,2-diamine and 28.9 g (0.28 mol) of sodium bisulfite in 200 ml of N,N-dimethylacetamide were heated to 100° C. 23.9 g (0.28 mol) of pivalaldehyde in 50 ml of N,N-dimethylacetamide were dropwise added during 10 minutes at 100° C., and heating continued during 20 minutes. The reaction mixture was poured into 1000 ml of water, and the resulting suspension filtered and washed with 1000 ml of water to give 46.8 g (97% yield) of Intermediate 22-1.

¹H NMR (400 MHz, DMSO-d₆) δ 12.06 (s, 1H), 7.53 (d, 1H), 7.41 (d, 1H), 7.17-7.05 (m, 2H), 1.40 (s, 9H).

### Intermediate 22-2

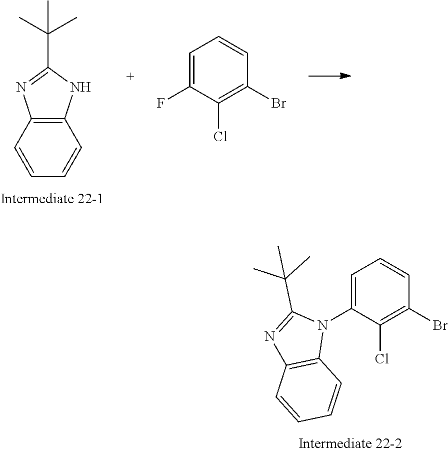 [SMILES: CC(C)(C)c1nc2ccccc2[nH]1 ; Fc1cccc(Br)c1Cl ; CC(C)(C)c1nc2ccccc2n1-c1cccc(Br)c1Cl]

20.0 g (115 mmol) of Intermediate 22-1, 48.1 g (230 mmol) of 1-bromo-2-chloro-3-fluoro-benzene, and 97.0 g (459 mmol) of potassium phosphate were suspended in 124 ml of 1,3-dimethylimidazolidin-2-one, followed by heating at 172° C. during 16 hours. The reaction mixture was cooled down to room temperature and treated with 1000 ml of water and 300 ml of heptane. The resulting suspension was filtered and the solid washed with 1000 ml of water and 300 ml of heptane. The solid was dissolved in 100 ml of dichloromethane and filtered through a 3 cm layer of silica gel and the silica gel layer washed with 200 ml of a 1:1 mixture of dichloromethane and ethyl acetate. The collected eluents were concentrated under vacuum to give 23.1 g (55% yield) of Intermediate 22-2.

¹H NMR (400 MHz, CD₂Cl₂) δ 7.92 (dd, 1H), 7.74 (d, 1H), 7.54 (dd, 1H), 7.42 (t, 1H), 7.29 (ddd, 1H), 7.20 (ddd, 1H), 6.76 (d, 1H), 1.38 (s, 9H).

### Intermediate 22-3

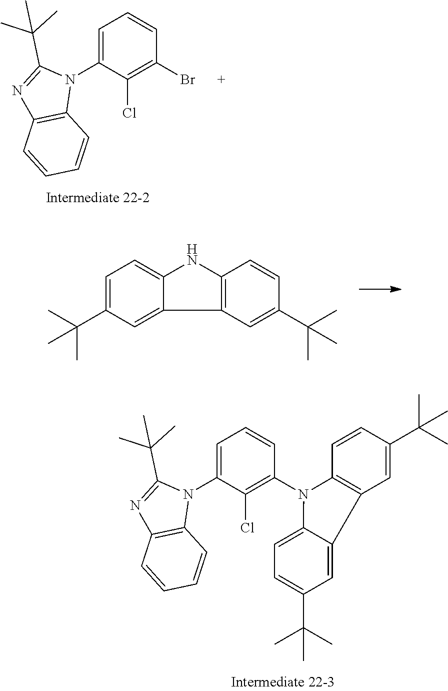

22.0 g (60.5 mmol) of Intermediate 22-2, 16.9 g (60.5 mmol) of 3,6-di-tert-butyl-9H-carbazole, 1.66 g (1.82 mmol) of tris(dibenzylideneacetone)dipalladium(0), 2.10 g (3.63 mmol) of 4,5-bis(diphenylphosphino)-9,9-dimethylxanthene (Xantphos), and 14.5 g (151 mmol) of sodium tert-butoxide were suspended in 250 ml of o-xylene. The suspension was three times evacuated and backfilled with argon and heated at 144° C. during 17 hours. The dark suspension was filtered through a 3 cm layer of silica gel followed by rinsing the silica gel layer with 100 ml of toluene. The eluent was concentrated under vacuum and the residue suspended in 200 ml of dichloromethane and 200 ml of ethanol. The suspension was concentrated to a volume of 300 ml, followed by filtration to give 27.5 g (81% yield) of Intermediate 22-3 as a white solid.

¹H NMR (400 MHz, CD₂Cl₂) δ 8.27-8.17 (d, 2H), 7.83-7.74 (m, 4H), 7.54 (ddd, 2H), 7.35-7.22 (m, 2H), 7.12 (d, 1H), 7.02 (d, 1H), 6.94-6.90 (m, 1H), 1.49 (s, 27H).

### Compound 22

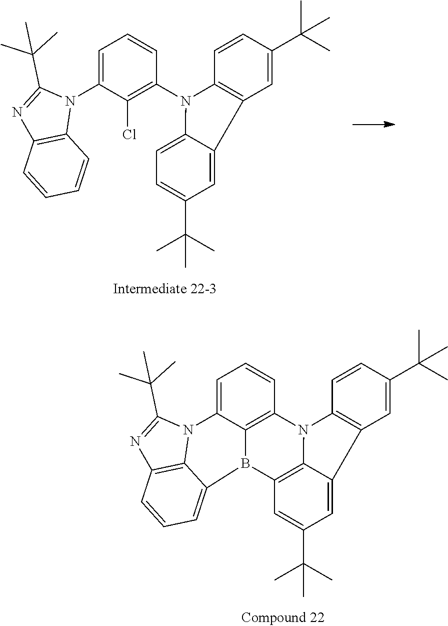

5.00 g (8.89 mmol) of Intermediate 22-3 were dissolved in 45 ml of water-free tert-butylbenzene. 9.36 ml of tert-butyllithium (1.9 M in pentane) were slowly added at −8° C. The solution was heated up to 68° C. and pentane distilled off. Heating was continued up to 72° C. during 10 minutes. The light yellow solution was cooled down to −56° C. and 1.71 ml (17.8 mmol) of tribromoborane were slowly added. The reaction mixture was stirred at room temperature during 15 minutes and cooled down to −3° C. 3.11 ml (17.8 mmol) of N,N-diiso-propylethylamine were added and the reaction mixture heated up to 154° C. during 53 hours. The dark suspension was treated with 20 ml of 10% aqueous sodium acetate solution and extracted with 30 ml of toluene. The organic layer was separated, washed with water (3×50 ml), then dried over sodium sulfate, and concentrated under vacuum. The resin was purified by MPLC with the CombiFlash Companion (silica gel, heptane/dichloromethane) to give 82 mg (2% yield) of Compound 22 as a yellow solid.

¹H NMR (400 MHz, CD₂Cl₂) δ 9.07 (d, 1H), 8.82 (dd, 1H), 8.59 (d, 1H), 8.53 (d, 1H), 8.42 (d, 1H), 8.35 (d, 1H), 8.25 (d, 1H), 8.10-8.02 (m, 2H), 7.76-7.66 (m, 2H), 1.93 (s, 9H), 1.69 (s, 9H), 1.57 (s, 9H).

### Compound 23

### Intermediate 23-1

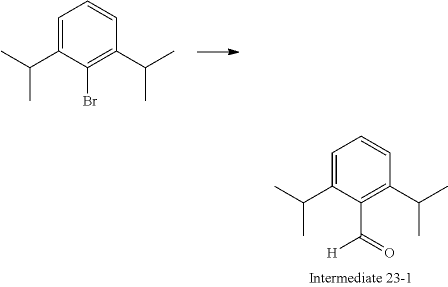

25.0 g (104 mmol) of 2-bromo-1,3-diisopropylbenzene were dissolved in 100 ml of tetrahydrofuran and cooled down to −78° C. 42.2 ml of n-butyllithium (2.7 M in heptane) were slowly added, followed by slow addition of 8.83 ml (114 mmol) of N,N-dimethylformamide at a maximum temperature of −65° C. The solution was warmed up to −16° C. during 45 minutes, then slowly treated with 20 ml of water and extracted with 50 ml of hexane. The organic phase was separated, dried over sodium sulfated and further concentrated under vacuum.

The product was purified by MPLC with the CombiFlash Companion (silica gel, heptane/ethyl acetate) to give 17.3 g (88% yield) of Intermediate 23-1.

¹H NMR (300 MHz, CDCl₃) δ 10.74 (s, 1H), 7.46 (dd, 1H), 7.28 (d, 2H), 3.63-3.46 (m, 2H), 1.30 (d, 12H).

### Intermediate 23-2

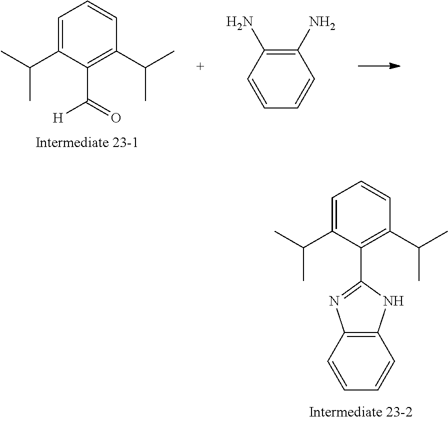 [SMILES: CC(C)c1cccc(C(C)C)c1C=O ; Nc1ccccc1N ; CC(C)c1cccc(C(C)C)c1-c1nc2ccccc2[nH]1]

7.39 g (68.3 mmol) of benzene-1,2-diamine and 7.11 g (68.3 mmol) of sodium bisulfite in 70 ml of N,N-dimethylacetamide were heated to 97° C. 13.0 g (68.3 mmol) of Intermediate 23-1 in 30 ml of N,N-dimethylacetamide were dropwise added during 15 minutes at 103° C., and heating continued during 4 hours. The reaction mixture was poured into 150 ml of water, and the resulting yellow suspension filtered and the solid washed with 100 ml of water. The solid was suspended in 100 ml of heptane and washed with 100 ml of heptane to give 16.6 g (87% yield) of Intermediate 23-2.

¹H NMR (400 MHz, DMSO-d₆) δ 12.53 (s, 1H), 7.77-7.38 (m, 3H), 7.30 (d, 2H), 7.26-7.15 (m, 2H), 2.48-2.35 (m, 2H), 1.10 (d, 12H).

### Intermediate 23-3

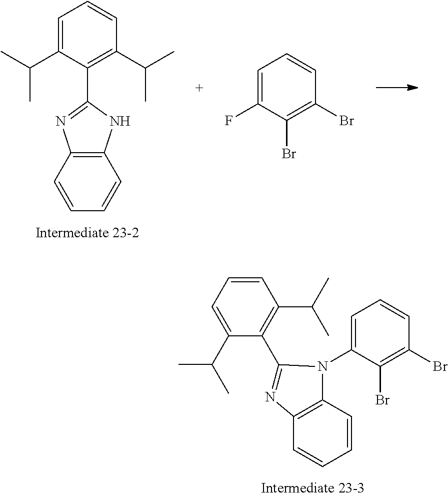 [SMILES: CC(C)c1cccc(C(C)C)c1-c1nc2ccccc2[nH]1 ; Fc1cccc(Br)c1Br ; CC(C)c1cccc(C(C)C)c1-c1nc2ccccc2n1-c1cccc(Br)c1Br]

6.70 g (24.1 mmol) of Intermediate 23-2, 6.72 g (26.2 mmol) of 2,3-dibromofluorobenzene, and 20.4 g (96 mmol) of potassium phosphate were suspended in 100 ml of N,N-dimethylformamide, followed by heating at 142° C. during 3 hours. The reaction mixture was cooled down to room temperature and poured into 200 ml of water. The resulting suspension was filtered and the solid washed with water. The solid was further dissolved in a mixture of 100 ml of 2-propanol and 50 ml of dichloromethane. The solution was concentrated under vacuum until a suspension formed. The suspension was filtered and the solid washed with 2-propanol to give 7.76 g (63% yield) of Intermediate 23-3.

¹H NMR (300 MHz, CD₂Cl₂) δ 7.94-7.86 (m, 1H), 7.71 (dd, 1H), 7.46-7.31 (m, 3H), 7.26 (dd, 1H), 7.21-7.01 (m, 4H), 2.82-2.64 (hept, 1H), 2.62-2.44 (hept, 1H), 1.32 (dd, 3H), 1.28 (dd, 3H), 1.09 (d, 3H), 0.89 (d, 3H).

### Intermediate 23-4

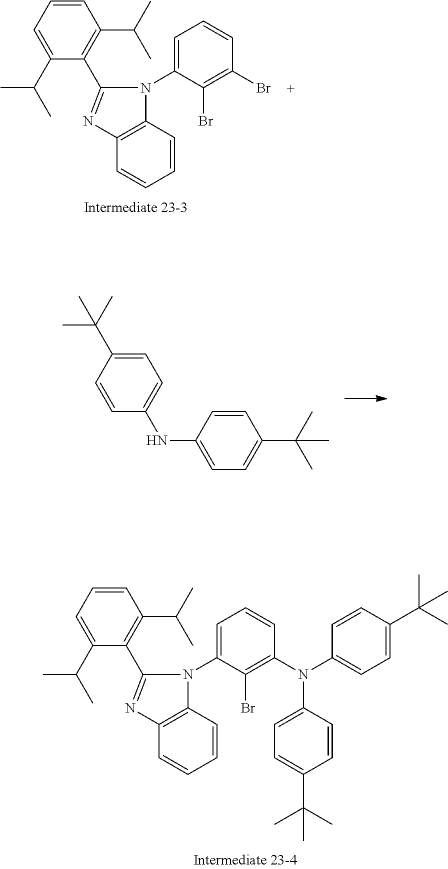 [SMILES: CC(C)c1cccc(C(C)C)c1-c1nc2ccccc2n1-c1cccc(Br)c1Br ; CC(C)(C)c1ccc(Nc2ccc(C(C)(C)C)cc2)cc1 ; CC(C)c1cccc(C(C)C)c1-c1nc2ccccc2n1-c1cccc(N(c2ccc(C(C)(C)C)cc2)c2ccc(C(C)(C)C)cc2)c1Br]

7.70 g (15.0 mmol) of Intermediate 23-3, 4.44 g (15.8 mmol) of bis(4-(tert-butyl)phenyl)amine, 275 mg (0.30 mmol) of tris(dibenzylideneacetone)dipalladium(0), 349 mg (1.20 mmol) of tri-tert-butylphosphonium tetrafluoroborate, and 3.61 g (37.6 mmol) of sodium tert-butoxide were suspended in 100 ml of o-xylene. The suspension was three times evacuated and backfilled with argon and heated at 124° C. during 30 minutes. The dark suspension was cooled down to room temperature and filtered through a 3 cm layer of silica gel followed by rinsing the silica gel layer with 100 ml of toluene. The collected eluent was concentrated under vacuum and purified by MPLC with the CombiFlash Companion (silica gel, dichloromethane). The resulting yellow solid was suspended in 50 ml of methanol and stirred during 1 hour. The suspension was filtered and the solid washed with 50 ml of methanol to give 5.70 g (53% yield) of Intermediate 23-4.

¹H NMR (400 MHz, CD₂Cl₂) δ 7.88 (d, 1H), 7.45 (t, 1H), 7.40-7.24 (m, 9H), 7.17 (d, 1H), 7.10 (d, 1H), 7.02 (m, 1H), 6.87 (d, 4H), 2.83-2.68 (m, 1H), 2.64-2.50 (m, 1H), 1.34 (s, 18H), 1.33 (d, 3H), 1.28 (d, 3H), 1.07 (d, 3H), 0.87 (d, 3H).

### Compound 23

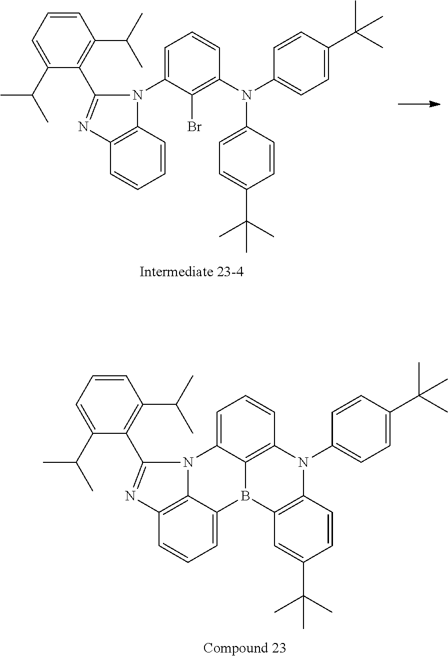

2.00 g (2.81 mmol) of Intermediate 23-4 were dissolved in 40 ml of water-free tert-butylbenzene. 1.48 ml of tert-butyllithium (1.9 M in pentane) were slowly added at −3° C. and stirred at −1° C. during 45 minutes. 1.48 ml of tert-butyllithium (1.9 M in pentane) were slowly added at 0° C. and stirring continued at 0° C. during 30 minutes. The yellowish solution was cooled down to −42° C. and 0.53 ml (5.61 mmol) of tribromoborane were added. The reaction mixture was warmed up to room temperature during 10 minutes and cooled down to 0° C. 0.98 ml (5.61 mmol) of N,N-diisopropylethylamine were added and the reaction mixture heated up to 157° C. during 22 hours. The yellowish reaction mixture was treated with 20 ml of water and 100 ml of heptane and stirred during 30 minutes over an ice bath. The suspension was filtered and the solid washed with heptane and the solid further purified by MPLC with the CombiFlash Companion (silica gel, dichloromethane/methanol) to give 270 mg of Compound 23 as a yellow solid.

¹H NMR (400 MHz, CD₂Cl₂) δ 9.13 (d, 1H), 8.85 (dd, 1H), 8.14 (d, 1H), 7.83-7.71 (m, 3H), 7.71-7.60 (m, 2H), 7.44 (d, 2H), 7.36-7.28 (m, 2H), 7.21 (t, 1H), 6.83 (d, 1H), 6.69 (d, 1H), 6.55 (d, 1H), 2.68-2.54 (m, 2H), 1.50 (s, 18H), 1.20 (d, 6H), 1.02 (d, 6H).

### Compound 24

### Intermediate 24-1

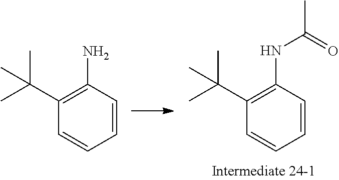

74.6 g (0.50 mol) of 2-(tert-butyl)aniline were dissolved in 650 ml of dichloromethane and dropwise treated with 61.3 g (0.60 mol) of acetic anhydride at a maximum temperature of 25° C. The solution was stirred during 3 hours at room temperature and treated with 800 ml of 10% aqueous sodium carbonate solution. The aqueous layer was separated and extracted with 200 ml of dichloromethane. The combined organic layers were washed with 500 ml of water, dried over magnesium sulfate and concentrated under vacuum. The product was heated in 500 ml of heptane under reflux during 15 minutes. The resulting suspension was filtered and the solid washed with 50 ml of heptane to give 94.4 g (98% yield) of Intermediate 24-1.

¹H NMR (400 MHz, CDCl₃) δ 7.57-7.37 (m, 2H), 7.36-7.03 (m, 3H), 2.22 (s, 3H), 1.43 (s, 9H).

### Intermediate 24-2

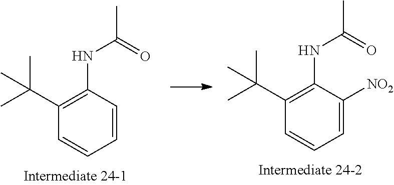 [SMILES: CC(=O)Nc1ccccc1C(C)(C)C ; CC(=O)Nc1c([N+](=O)[O-])cccc1C(C)(C)C]

4.78 g (25 mmol) of Intermediate 24-1, 17.5 ml of acetic acid and 17.5 ml of acetic anhydride were heated up to 40° C. 2.42 g of 65% nitric acid were dropwise added during 5 minutes and the solution stirred at 50° C. during 90 minutes. The reaction mixture was poured into 250 ml water, followed by the addition of 200 g of ice and 200 ml of tert-butyl methyl ether. 64 g of sodium carbonate were slowly added. The aqueous phase was separated and extracted with 100 ml of tert-butyl methyl ether. The combined organic phases were washed with saturated aqueous sodium chloride solution, dried over magnesium sulfate and concentrated under vacuum. The product was further purified by MPLC with the CombiFlash Companion (silica gel, heptane/20-40% gradient of ethyl acetate) to give 1.20 g (20% yield) of Intermediate 24-2 as a beige solid.

¹H NMR (300 MHz, DMSO-d₆) δ 9.63 (s, 1H), 7.78-7.71 (m, 2H), 7.48 (t, 1H), 2.01 (s, 3H), 1.36 (s, 9H).

### Intermediate 24-3

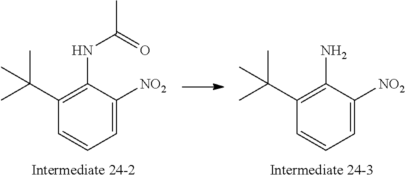 [SMILES: CC(=O)Nc1c([N+](=O)[O-])cccc1C(C)(C)C ; CC(C)(C)c1cccc([N+](=O)[O-])c1N]

A solution of 19.4 g (82 mmol) of Intermediate 24-2, 88 g (172 mmol) of potassium hydroxide, 63 ml of water and 250 ml of methanol was heated at 100° C. during 44 hours. The reaction mixture was cooled down and poured into 220 ml of water, followed by stirring at room temperature during 5 minutes. The suspension was filtered, and the solid washed with water (3×25 ml) giving 11.5 g of an orange solid. The filtrate was extracted with ethyl acetate (2×150 ml) and the combined organic phases washed with 200 ml of saturated aqueous sodium chloride solution, dried over magnesium sulfated and concentrated under vacuum, giving 4.8 g of a solid. The two solid products were combined and further purified by MPLC with the CombiFlash Companion (silica gel, heptane/5-15% gradient of ethyl acetate) to give 11.8 g (73% yield) of Intermediate 24-3 as an orange solid.

¹H NMR (400 MHz, DMSO-d₆) δ 7.93 (dd, 1H), 7.46 (dd, 1H), 7.13 (s, 2H), 6.63 (dd, 1H), 1.40 (s, 9H).

### Intermediate 24-4

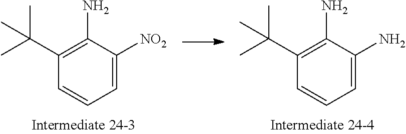

11.3 g (58 mmol) of Intermediate 24-3 and 1.16 g (29 mmol) of sodium hydroxide in 70 ml of ethanol were heated up to reflux temperature. The red suspension was treated in ten portions with 15.2 g (0.23 mol) of zinc dust during 10 minutes, and stirring continued at reflux temperature during three hours. The reaction mixture was filtered and the solid washed with ethanol (3×15 ml). The filtrate was concentrated under vacuum and 100 ml of ethyl acetate and 100 ml of water were added. The organic phase was separated and washed with water (2×100 ml), dried over magnesium sulfate and concentrated under vacuum to give 9.11 g (95%) of Intermediate 24-4 as an oil.

¹H NMR (400 MHz, DMSO-d₆) δ 6.58-6.49 (m, 2H), 6.40 (t, 1H), 4.32 (br. s, 2H), 4.06 (br. s, 2H), 1.36 (s, 9H).

### Intermediate 24-5

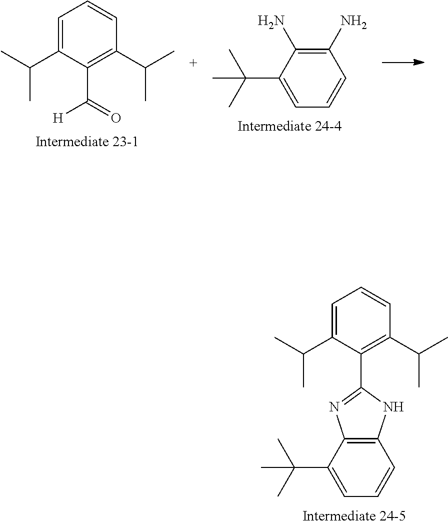 [SMILES: CC(C)c1cccc(C(C)C)c1C=O ; CC(C)(C)c1cccc(N)c1N ; CC(C)c1cccc(C(C)C)c1-c1nc2c(C(C)(C)C)cccc2[nH]1]

To 3.74 g (22.8 mmol) of Intermediate 24-4 in 40 ml of N,N-dimethylacetamide, 2.68 g (25.8 mmol) sodium bisulfite were added and heated to 100° C. 3.50 g (18.39 mmol) of Intermediate 23-1 in 25 ml of N,N-dimethylacetamide were added drop by drop at 100° C., then it was stirred at this temperature for 4 hours. The reaction mixture was cooled down to room temperature and poured on 100 ml of water and stirred. The suspension was filtrated and washed with 100 ml of water. The crude product was suspended in 100 ml of heptane, washed with 100 ml of heptane, filtrated and dried to give 5.05 g (82% yield) of Intermediate 24-5.

¹H NMR (300 MHz, DMSO-d₆) δ 12.35 (d, 1H), 7.53-7.45 (m, 1H), 7.35-7.26 (m, 3H), 7.16-7.07 (m, 1H), 7.03 (dd, 1H), 2.47-2.36 (m, 2H), 1.67 and 1.54 (2 s, 9H), 1.22-1.00 (br. m, 12H).

### Intermediate 24-6

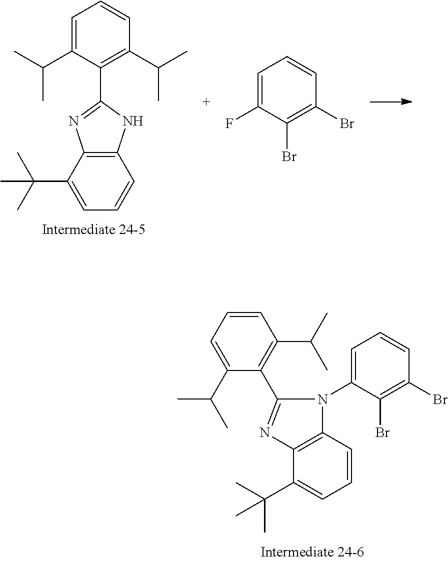 [SMILES: CC(C)c1cccc(C(C)C)c1-c1nc2c(C(C)(C)C)cccc2[nH]1 ; Fc1cccc(Br)c1Br ; CC(C)c1cccc(C(C)C)c1-c1nc2c(C(C)(C)C)cccc2n1-c1cccc(Br)c1Br]

5.55 g (16.61 mmol) of Intermediate 24-5 and 4.80 g (18.91 mmol) of 1,2-dibromo-3-fluorobenzene were dissolved in 80 ml of N,N-dimethylformamide, then 17.62 g (83 mmol) of potassium phosphate tribasic were added. The reaction mixture was heated to 150° C. (outside temperature) for 2 hours, then cooled down to room temperature and the salt was filtered off. The solvent of the filtrate was removed at reduced pressure. The isolated solid was dissolved in 50 ml of dichloromethane and 30 ml of heptane were added. The solution was concentrated under vacuum to a volume of 30 ml and the resulting suspension was filtered and the solid washed with heptane. The crude product was purified by column chromatography (heptane/ethylacetate) to give 7.2 g (85% yield) of Intermediate 24-6.

¹H NMR (300 MHz, CD₂Cl₂) δ 7.67 (dd, 1H), 7.36 (t, 1H), 7.25-7.14 (m, 3H), 7.13-7.02 (m, 3H), 6.97 (dd, 1H), 2.73 (p, 1H), 2.54 (p, 1H), 1.64 (s, 9H), 1.26 (dd, 6H), 1.05 (d, 3H), 0.89 (d, 3H).

### Intermediate 24-7

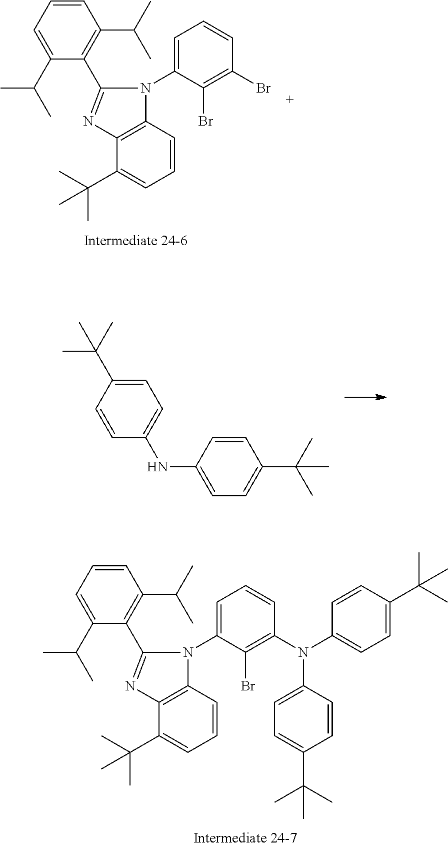 [SMILES: CC(C)c1cccc(C(C)C)c1-c1nc2c(C(C)(C)C)cccc2n1-c1cccc(Br)c1Br ; CC(C)(C)c1ccc(Nc2ccc(C(C)(C)C)cc2)cc1 ; CC(C)c1cccc(C(C)C)c1-c1nc2c(C(C)(C)C)cccc2n1-c1cccc(N(c2ccc(C(C)(C)C)cc2)c2ccc(C(C)(C)C)cc2)c1Br]

8.00 g (14.1 mmol) of Intermediate 24-6, 4.16 g (14.8 mmol) of bis(4-(tert-butyl)phenyl)amine, 258 mg (0.28 mmol) of tris(dibenzylideneacetone)dipalladium(0), 327 mg (1.13 mmol) of tri-tert-butylphosphonium tetrafluoroborate, and 3.38 g (35.2 mmol) of sodium tert-butoxide were suspended in 100 ml of o-xylene. The suspension was 3 times evacuated and backfilled with argon and heated at 124° C. during 30 minutes. The dark suspension was cooled down to room temperature and filtered through a 3 cm layer of silica gel followed by rinsing the silica gel layer with 200 ml of toluene. The collected eluent was concentrated under vacuum and purified by MPLC with the CombiFlash Companion (silica gel, dichloromethane). The isolated solid was dissolved in 20 ml of dichloromethane and diluted with 100 ml of ethanol. Dichloromethane was removed under vacuum and the suspension stirred during 2 hours. The suspension was filtered and the solid washed with 50 ml of ethanol. The precipitation was repeated with 30 ml of dichloromethane and 100 ml of ethanol and the resulting solid several times washed with ethanol to give 3.16 g (29% yield) of Intermediate 24-7.

¹H NMR (400 MHz, CD₂Cl₂) δ 7.45 (t, 1H), 7.33-7.22 (m, 9H), 7.17 (dd, 1H), 7.08-7.01 (m, 1H), 6.96 (dd, 1H), 6.90-6.83 (m, 4H), 2.79 (hept, 1H), 2.60 (hept, 1H), 1.67 (s, 9H), 1.34 (s, 18H), 1.33 (d, 3H), 1.28 (d, 3H), 1.07 (d, 3H), 0.87 (d, 3H).

### Compound 24

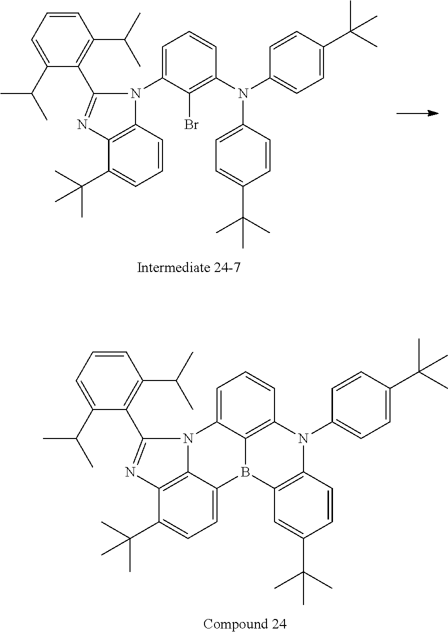

4.00 g (5.20 mmol) of Intermediate 24-7 were dissolved in 50 ml of water-free tert-butylbenzene. 5.48 ml of tert-butyllithium (1.9 M in pentane) were slowly added at −6° C. and heated up to 51° C. during 35 minutes. The yellowish solution was cooled down to −51° C. and 0.98 ml (10.4 mmol) of tribromoborane were added. The reaction mixture was warmed up to room temperature during 10 minutes and cooled down to −2° C. 1.81 ml (10.4 mmol) of N,N-diisopropylethylamine were added and the reaction mixture heated up to 152° C. during 22 hours. The yellow suspension was cooled down and treated with 30 ml of 10% aqueous sodium acetate solution and extracted with 100 ml of heptane. The organic layer was separated, washed with water (3×50 ml), then dried over sodium sulfate, and concentrated under vacuum. The isolated product was purified by MPLC with the CombiFlash Companion (silica gel, heptane/dichloromethane). The isolated solid was dissolved in 50 ml of dichloromethane and 100 ml of ethanol were added. The solution was concentrated under vacuum to a volume of 100 ml and the resulting suspension filtered and the solid washed with ethanol. The solid was further suspended in a mixture of 30 ml of acetonitrile and 10 ml of dichloromethane during 30 minutes. The suspension was filtered and the solid washed with 20 ml of acetonitrile. The solid was dissolved in 20 ml of dichloromethane and mixed with 50 ml of 2-propanol. The solution was concentrated under vacuum to a volume of 50 ml. The resulting suspension was filtered and the solid washed with 2-propanol to give 1.28 g (35% yield) of Compound 24 as a yellow solid.

¹H NMR (400 MHz, CD₂Cl₂) δ 9.11 (d, 1H), 8.78 (d, 1H), 7.79-7.72 (m, 2H), 7.72-7.61 (m, 3H), 7.44 (d, 2H), 7.33-7.27 (m, 2H), 7.21 (t, 1H), 6.81 (dd, 2H), 6.54 (d, 1H), 2.65-2.51 (m, 2H), 1.77 (s, 9H), 1.53 (s, 9H), 1.50 (s, 9H), 1.21 (d, 6H), 1.05 (d, 6H).

### Compound 25

### Intermediate 25-1

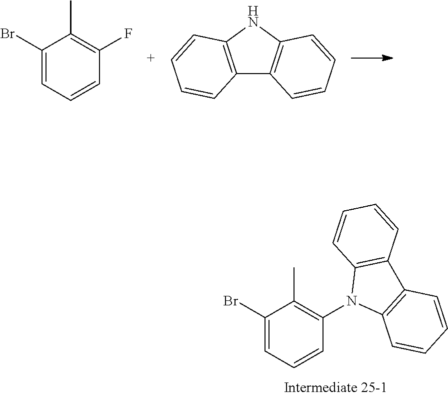

15.0 g (90 mmol) of 9H-carbazole and 51.0 g (270 mmol) of 1-bromo-3-fluoro-2-methylbenzene were dissolved in 350 ml of N,N-dimethylformamide. 66.60 g (314 mmol) of potassium phosphate tribasic were added. The reaction mixture was heated at 150° C. (outside temperature) for 40 hours, then cooled to room temperature and filtered. The solvent of the filtrate was removed at reduced pressure. The residue was taken in ethyl acetate, washed several times with water, dried over magnesium sulfate, filtered and concentrated under vacuum. The crude product was suspended in methanol and stirred for 1 hour, then filtered, washed with methanol and heptane and dried to give 28.7 g (95% yield) of Intermediate 25-1.

¹H NMR (300 MHz, Chloroform-d) δ 8.17 (dt, J=7.7, 1.0 Hz, 2H), 7.76 (dd, J=7.9, 1.5 Hz, 1H), 7.45-7.27 (m, 6H), 7.04 (dt, J=8.1, 0.9 Hz, 2H), 2.04 (s, 3H).

### Intermediate 25-2

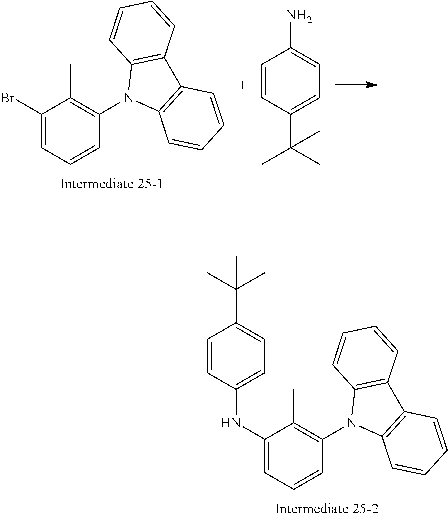 [SMILES: Cc1c(Br)cccc1-n1c2ccccc2c2ccccc21 ; CC(C)(C)c1ccc(N)cc1 ; Cc1c(Nc2ccc(C(C)(C)C)cc2)cccc1-n1c2ccccc2c2ccccc21]

8.01 g (53.7 mmol) of 4-(tert-butyl)aniline, 16.4 g (48.8 mmol) of Intermediate 25-1 and 14.06 g (146 mmol) of sodium tert-butoxide were suspended in 400 ml of toluene. The mixture was evacuated and backfilled with argon 4 times, then 1.240 g (1.951 mmol) of BINAP and 1.191 g (0.976 mmol) of Pd₂(dba)₃ were added. The reaction mixture was heated at 110° C. for 3 hours, then cooled to room temperature. 100 ml of water were added and stirred. The phases were separated, the water phase was extracted with ethyl acetate, the combined organic phases were washed with water and brine, dried over magnesium sulfate, filtered and concentrated under vacuum. The crude product was suspended in methanol and stirred at 60° C. for 1 hour, then the precipitate was filtered and washed with heptane. The product was further purified by column chromatography (heptane/ethyl acetate) to give 19.87 g (99% yield) of Intermediate 25-2.

¹H NMR (300 MHz, Methylene Chloride-d2) δ 8.17 (dt, J=7.7, 1.0 Hz, 2H), 7.46-7.33 (m, 5H), 7.33-7.23 (m, 3H), 7.15-7.04 (m, 4H), 6.98 (dd, J=7.6, 1.4 Hz, 1H), 5.58 (s, 1H), 1.81 (s, 3H), 1.34 (s, 9H).

### Intermediate 25-3

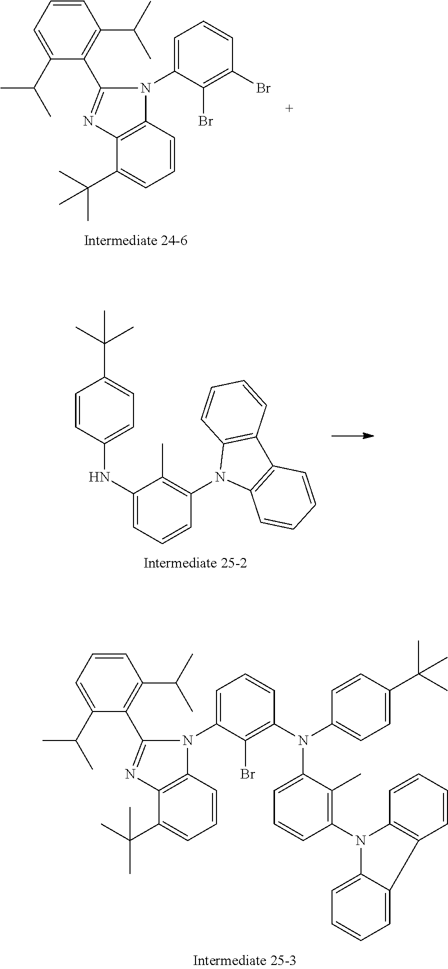 [SMILES: CC(C)c1cccc(C(C)C)c1-c1nc2c(C(C)(C)C)cccc2n1-c1cccc(Br)c1Br ; Cc1c(Nc2ccc(C(C)(C)C)cc2)cccc1-n1c2ccccc2c2ccccc21 ; Cc1c(N(c2ccc(C(C)(C)C)cc2)c2cccc(-n3c(-c4c(C(C)C)cccc4C(C)C)nc4c(C(C)(C)C)cccc43)c2Br)cccc1-n1c2ccccc2c2ccccc21]

6.0 g (8.97 mmol) of Intermediate 24-6, 3.63 g (8.97 mmol) of Intermediate 25-2 and 2.15 g (22.43 mmol) of sodium tert-butoxide were suspended in 110 ml of xylene. The mixture was evacuated and backfilled with argon 4 times. 0.213 g (0.718 mmol) of tri-tert-butylphosphonium tetrafluoroborate and 0.164 g (0.179 mmol) of Pd₂(dba)₃ were added and the reaction mixture was heated at 115° C. for 1.5 hour, then cooled to room temperature. 30 ml of water were added and stirred. The phases were separated, the water phase was extracted with ethyl acetate, the combined organic phases were washed with water and brine, dried over magnesium sulfate, filtered and the solvent was evaporated under reduced pressure. The crude product was purified by column chromatography (heptane/dichloromethane) to give 3.96 g (47% yield) of Intermediate 25-3.

¹H NMR (300 MHz, Methylene Chloride-d2) δ 8.18 (dq, J=7.8, 0.9 Hz, 2H), 7.47-7.35 (m, 4H), 7.34-7.21 (m, 10H), 7.21-7.13 (m, 2H), 7.10-6.99 (m, 4H), 6.91 (dd, J=7.4, 1.7 Hz, 1H), 6.78 (d, J=8.3 Hz, 2H), 2.83 (p, J=6.7 Hz, 1H), 2.62 (p, J=6.8 Hz, 1H), 1.67 (s, 9H), 1.64 (d, J=3.2 Hz, 3H), 1.34 (d, J=1.4 Hz, 11H), 1.26 (d, J=6.7 Hz, 3H), 1.08 (d, J=6.7 Hz, 3H), 0.90 (d, J=6.8 Hz, 3H).

### Compound 25

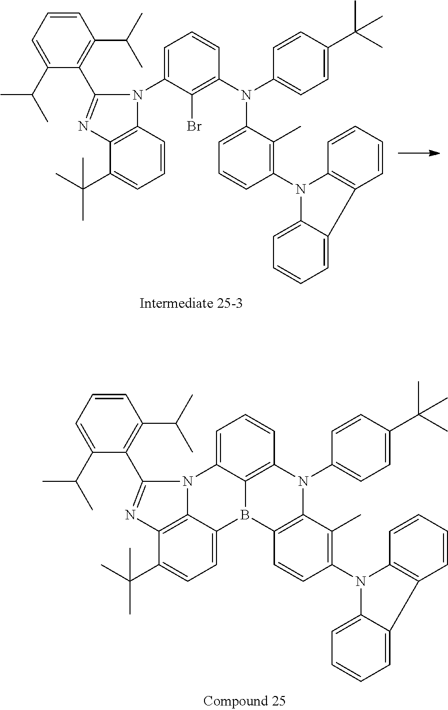 [SMILES: Cc1c(N(c2ccc(C(C)(C)C)cc2)c2cccc(-n3c(-c4c(C(C)C)cccc4C(C)C)nc4c(C(C)(C)C)cccc43)c2Br)cccc1-n1c2ccccc2c2ccccc21 ; Cc1c(-n2c3ccccc3c3ccccc32)ccc2c1N(c1ccc(C(C)(C)C)cc1)c1cccc3c1B2c1ccc(C(C)(C)C)c2nc(-c4c(C(C)C)cccc4C(C)C)n-3c12]

2.72 g (3.0 mmol) of Intermediate 25-3 were dissolved in 80 ml of water-free tert-butylbenzene. 2.50 ml (4.75 mmol) of tert-butyllithium (1.9 M in pentane) were slowly added at −15° C., then it was heated up to room temperature and stirred for 4 hours. The yellowish solution was cooled down to −34° C. and 1.15 ml (12.2 mmol) of tribromoborane were added. The reaction mixture was warmed up to room temperature, stirred for 1 hour and then cooled down to 0° C. 2.15 ml (12.3 mmol) of N,N-diisopropylethylamine were added and the reaction mixture was heated at 152° C. for 20 hours. The yellow suspension was cooled down and treated with 30 ml of 10% aqueous sodium acetate solution and extracted with 100 ml of ethyl acetate. The organic layer was separated, washed with water, then dried over sodium sulfate, filtered and concentrated under vacuum. The isolated product was purified by column chromatography (heptane/ethyl acetate). The product was dissolved in 40 ml of dichloromethane and 80 ml of 2-propanol were added. The solution was concentrated under vacuum to a volume of 80 ml and the resulting suspension was filtered and the solid washed with 2-propanol. This procedure was repeated twice. The solid was further suspended in a mixture of 30 ml of acetonitrile and 10 ml of dichloromethane and stirred for 30 minutes. The suspension was filtered, the solid washed with acetonitrile and dried to give 0.48 g (19% yield) of Compound 25 as a yellow solid.

¹H NMR (300 MHz, ) δ 9.13 (d, J=2.4 Hz, 1H), 8.74 (d, J=8.0 Hz, 1H), 8.17 (ddt, J=7.8, 2.2, 1.0 Hz, 2H), 7.81-7.58 (m, 5H), 7.54-7.38 (m, 5H), 7.35-7.25 (m, 3H), 7.19 (ddt, J=14.1, 8.1, 0.9 Hz, 2H), 6.92-6.79 (m, 2H), 6.58 (dd, J=8.6, 0.7 Hz, 1H), 2.56 (pd, J=6.8, 2.2 Hz, 2H), 1.73 (s, 9H), 1.53 (s, 9H), 1.50 (s, 3H), 1.16 (dd, J=6.8, 4.4 Hz, 6H), 1.02 (dd, J=11.1, 6.9 Hz, 6H).

### Compound 26

### Intermediate 26-1

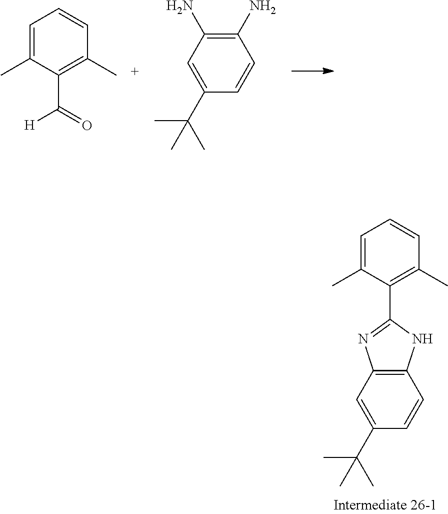

To 44.5 g (263 mmol) of 4-(tert-butyl)benzene-1,2-diamine in 250 ml of N,N-dimethylacetamide 30.8 g (296 mmol) of sodium bisulfite were added and heated to 100° C. 30.0 g (219 mmol) of 2,6-dimethylbenzaldehyde in 150 ml of N,N-dimethylacetamide were added dropwise at 100° C., then it was stirred at this temperature for 1 hour. The reaction mixture was cooled to room temperature and poured on 750 ml of water and stirred. The suspension was filtered and washed with 750 ml of water and 500 ml of heptane and dried. The isolated solid was suspended in 1l of dichloromethane and sonicated for 1 hour. The precipitate was filtered, washed with dichloromethane and dried to give 59 g (97% yield) of Intermediate 26-1.

¹H NMR (300 MHz, DMSO-d6) δ 12.33 (s, 1H), 7.67-7.55 (m, 1H), 7.43-7.38 (m, 1H), 7.34-7.25 (m, 2H), 7.18 (d, J=7.6 Hz, 2H), 2.09 (s, 6H), 1.36 (d, J=1.5 Hz, 9H).

### Intermediate 26-2

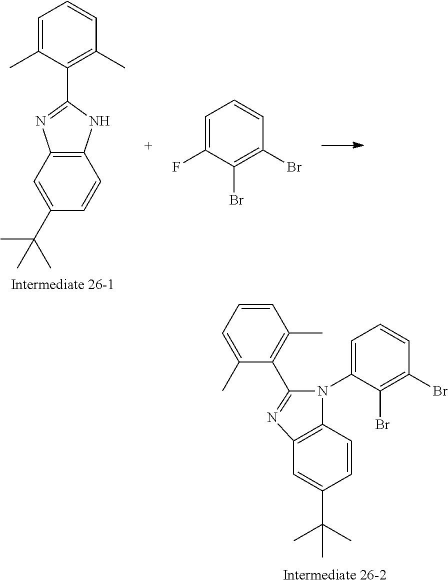 [SMILES: Cc1cccc(C)c1-c1nc2cc(C(C)(C)C)ccc2[nH]1 ; Fc1cccc(Br)c1Br ; Cc1cccc(C)c1-c1nc2cc(C(C)(C)C)ccc2n1-c1cccc(Br)c1Br]

32.36 g (116 mmol) of Intermediate 26-1 and 35.4 g (139 mmol) of 1,2-dibromo-3-fluorobenzene were dissolved in 300 ml of N,N-dimethylformamide. 123 g (581 mmol) of potassium phosphate tribasic were added. The reaction mixture was heated at 150° C. (outside temperature) for 1.5 hours, then cooled to room temperature and filtered. The filtrate was concentrated under vacuum and the isolated solid was dissolved in 100 ml of dichloromethane, then 80 ml of heptane were added. The solution was concentrated under vacuum to a volume of 80 ml, the resulting suspension was filtered, the solid washed with heptane and dried. The isomers were separated by column chromatography (dichloromethane) to give 16.06 g (28% yield) of Intermediate 26-2.

¹H NMR (300 MHz, Methylene Chloride-d2) δ 7.88 (dd, J=1.9, 0.7 Hz, 1H), 7.67 (dd, J=8.0, 1.6 Hz, 1H), 7.40 (dd, J=8.6, 1.9 Hz, 1H), 7.22-7.04 (m, 4H), 7.02 (dd, J=8.0, 1.6 Hz, 1H), 6.97 (dq, J=7.6, 1.4, 0.7 Hz, 1H), 2.22 (s, 3H), 2.09 (s, 3H), 1.44 (s, 9H).

### Intermediate 26-3

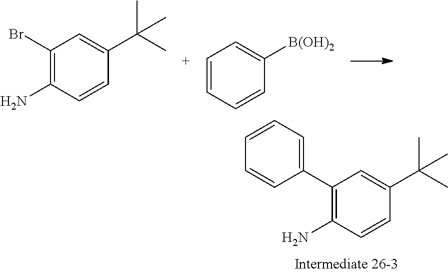

40 g (175 mmol) of 2-bromo-4-(tert-butyl)aniline, 26.2 g (210 mmol) of phenylboronic acid and 72.7 g (526 mmol) of potassium carbonate were suspended in 835 ml of toluene, 417 ml of tetrahydrofuran and 209 ml of water. The mixture was evacuated and backfilled with argon 4 times. 10.13 g (8.77 mmol) Pd(PPh₃)₄ were added and the reaction mixture was heated at 90° C. for 19 hours. The cooled reaction mixture was diluted with 250 ml of ethyl acetate, the phases were separated and the water phase extracted with ethyl acetate. The combined organic phases were washed with water and brine, dried over magnesium sulfate, filtered and concentrated under vacuum. The isolated product was purified by column chromatography (heptane/ethyl acetate) to give 25 g (63% yield) of Intermediate 26-3.

LC-MS: 224.1 [M−H]⁻

### Intermediate 26-4

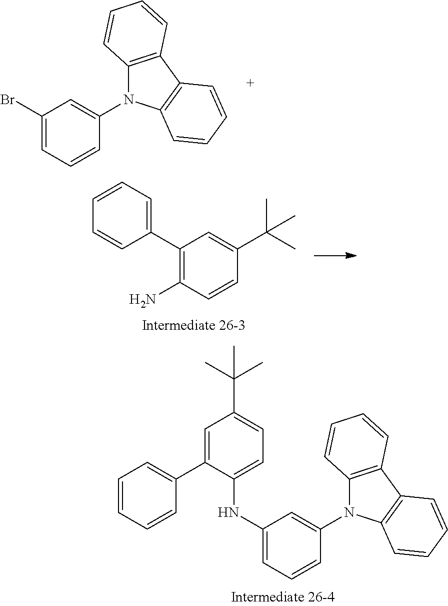

10.0 g (44.4 mmol) of Intermediate 26-3, 13.02 g (40.4 mmol) of 9-(3-bromophenyl)-9H-carbazole and 11.65 g (121 mmol) of sodium tert-butoxide were suspended in 150 ml of toluene. The mixture was evacuated and backfilled with argon 4 times, then 1.027 g (1.616 mmol) of BINAP and 0.987 g (0.808 mmol) Pd₂(dba)₃ were added. The reaction mixture was heated at 110° C. for 7 hours, then cooled to room temperature. 50 ml of water were added and stirred. The phases were separated, the water phase was extracted with ethyl acetate, the combined organic phases were washed with water and brine, dried over sodium sulfate, filtered and concentrated under vacuum. The crude product was purified by column chromatography (heptane/ethyl acetate) to give 15.3 g (81% yield) of Intermediate 26-4.

LC-MS: 465.2 [M−H]⁻

### Intermediate 26-5

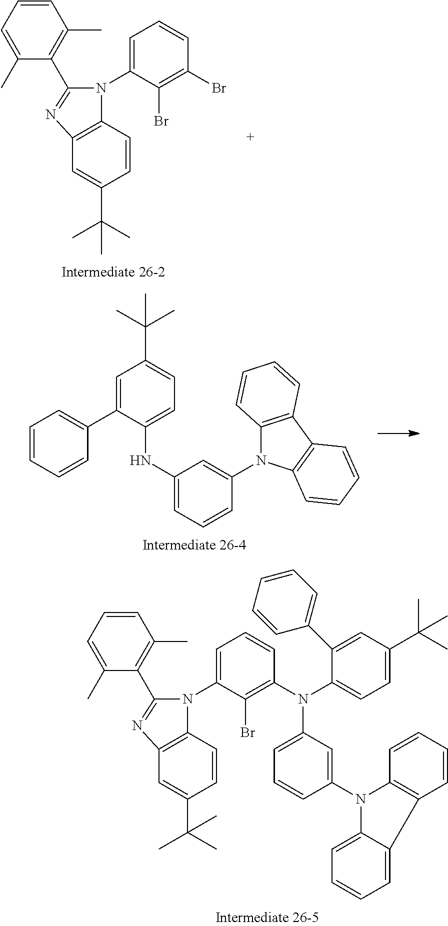 [SMILES: Cc1cccc(C)c1-c1nc2cc(C(C)(C)C)ccc2n1-c1cccc(Br)c1Br ; CC(C)(C)c1ccc(Nc2cccc(-n3c4ccccc4c4ccccc43)c2)c(-c2ccccc2)c1 ; Cc1cccc(C)c1-c1nc2cc(C(C)(C)C)ccc2n1-c1cccc(N(c2cccc(-n3c4ccccc4c4ccccc43)c2)c2ccc(C(C)(C)C)cc2-c2ccccc2)c1Br]

12.0 g (23.42 mmol) of Intermediate 26-2, 11.48 g (24.60 mmol) of Intermediate 26-4 and 5.63 g (58.6 mmol) of sodium tert-butoxide were suspended in 385 ml of toluene. The mixture was evacuated and backfilled with argon 4 times. 0.725 g (1.874 mmol) of tri-tert-butylphosphonium tetrafluoroborate and 0.438 g (0.468 mmol) of Pd₂(dba)₃ were added and the reaction mixture was heated at 85° C. for 2 hours, then cooled to room temperature. 80 ml of water were added and stirred. The phases were separated, the water phase was extracted with ethyl acetate, the combined organic phases were washed with water and brine, dried over magnesium sulfate, filtered and reduced under vacuum. The crude product was purified by column chromatography (heptane/ethyl acetate) to give 16.6 g (79% yield) of Intermediate 26-5.

¹H NMR (300 MHz, Methylene Chloride-d2) δ 8.14 (dt, J=7.7, 1.1 Hz, 2H), 7.78 (d, J=1.8 Hz, 1H), 7.45-7.16 (m, 19H), 7.13-7.05 (m, 1H), 7.04-6.87 (m, 4H), 6.68 (d, J=8.2 Hz, 2H), 2.10 (s, 6H), 1.42 (s, 9H), 1.35 (s, 9H).

### Compound 26

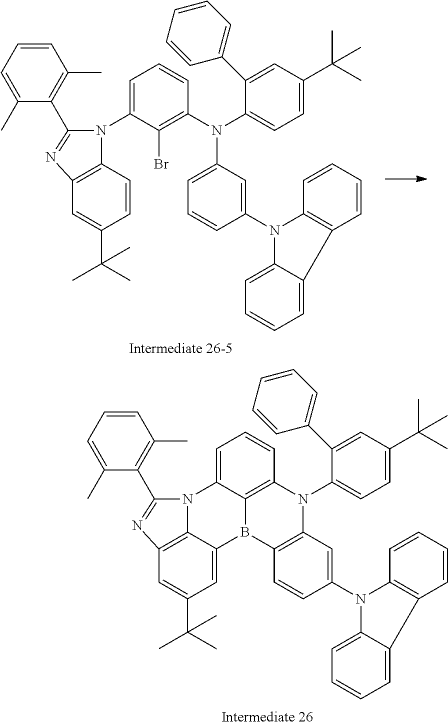

10.0 g (11.14 mmol) of Intermediate 26-5 were dissolved in 300 ml of water-free tert-butylbenzene. 13.10 ml (22.27 mmol) of tert-butyllithium (1.9 M in pentane) were slowly added at 0° C. and it was stirred at this temperature for 30 minutes. The yellowish solution was cooled down to −50° C. and 2.105 ml (22.27 mmol) of tribromoborane were added. The reaction mixture was warmed up to room temperature, stirred for 30 minutes and then cooled down to 0° C. 9.72 ml (55.7 mmol) of N,N-diisopropylethylamine were added and the reaction mixture was heated at 165° C. for 16 hours. 9.72 ml (55.7 mmol) of N,N-diisopropylethylamine were added again and the reaction mixture was heated at 165° C. for 7 hours, then 9.72 ml (55.7 mmol) of N,N-diisopropylethylamine were added and the reaction mixture was heated at 165° C. for another 16 hours. The yellow suspension was cooled down and treated with 120 ml of 10% aqueous sodium acetate solution and extracted with 400 ml of ethyl acetate. The organic layer was washed with water, then dried over magnesium sulfate, filtered and concentrated under vacuum. The isolated solid was purified by column chromatography (dichloromethane). The product was dissolved in 40 ml of dichloromethane, then 50 ml of methanol were added. The solution was concentrated under vacuum to a volume of 50 ml, the resulting suspension filtered and the solid washed with methanol and dried to give 1.60 g (17.3% yield) of Compound 26 as a yellow solid.

¹H NMR (300 MHz, Methylene Chloride-d2) δ 9.22 (d, J=8.3 Hz, 1H), 8.87 (d, J=1.7 Hz, 1H), 8.19 (d, J=1.6 Hz, 1H), 8.12 (dt, J=7.7, 1.1 Hz, 2H), 7.69-7.63 (m, 2H), 7.54 (dd, J=8.3, 1.9 Hz, 1H), 7.49-7.22 (m, 12H), 7.14-7.01 (m, 5H), 6.71-6.63 (m, 2H), 2.16 (s, 3H), 2.10 (s, 3H), 1.62 (s, 9H), 1.37 (s, 9H).

### Compound 27

### Intermediate 27-1

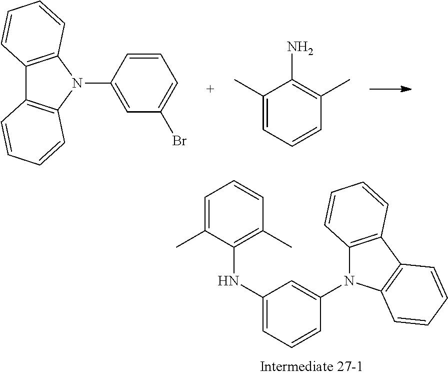

6.78 g (56.0 mmol) 2,6-dimethylaniline, 16.40 g (50.9 mmol) of 9-(3-bromophenyl)-9H-carbazole and 14.67 g (153 mmol) of sodium tert-butoxide were suspended in 400 ml of toluene. The mixture was evacuated and backfilled with argon 4 times, then 1.294 g (2.036 mmol) of BINAP and 1.243 g (1.018 mmol) of Pd₂(dba)₃ were added. The reaction mixture was heated at 110° C. for 1 hour, then cooled to room temperature. 100 ml of water were added and stirred. The phases were separated, the water phase was extracted with ethyl acetate, the combined organic phases were washed with water and brine, dried over magnesium sulfate, filtered and concentrated under vacuum. The crude product was purified by column chromatography (heptane/ethyl acetate). The product was suspended in ethanol, sonicated for 1 hour, filtered, washed with ethanol and dried to give 13.08 g (71% yield) of Intermediate 27-1.

¹H NMR (300 MHz, Methylene Chloride-d₂) δ 8.11 (dt, J=7.8, 1.1 Hz, 2H), 7.49-7.33 (m, 5H), 7.25 (ddd, J=8.0, 5.9, 2.2 Hz, 2H), 7.17-7.01 (m, 3H), 6.91 (ddd, J=7.7, 2.0, 1.0 Hz, 1H), 6.71-6.55 (m, 2H), 5.47 (s, 1H), 2.29 (s, 6H).

### Intermediate 27-2

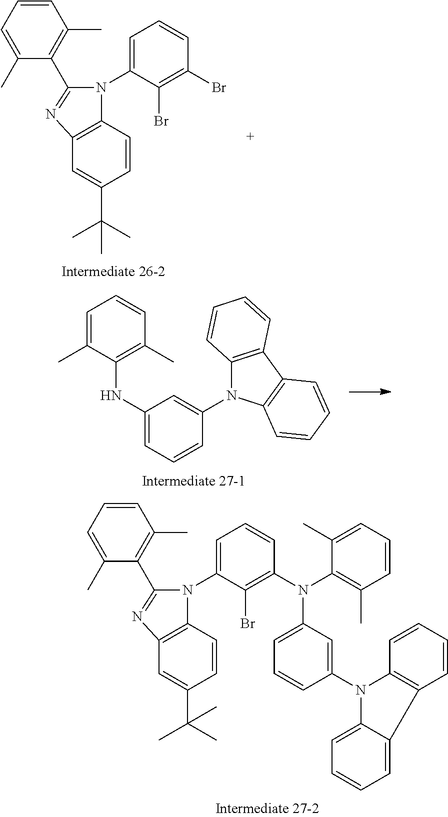

6.95 g (13.58 mmol) of Intermediate 26-2, 5.22 g (14.26 mmol) of Intermediate 27-1 and 3.91 g (40.7 mmol) of sodium tert-butoxide were suspended in 185 ml of toluene. The mixture was evacuated and backfilled with argon 4 times. 0.325 g (1.086 mmol) of tri-tert-butylphosphonium tetrafluoroborate and 0.332 g (0.272 mmol) of Pd₂(dba)₃ were added and the reaction mixture was heated at 100° C. for 2.5 hours, then cooled to room temperature. 60 ml of water were added and stirred. The phases were separated, the water phase was extracted with ethylaceteate, the combined organic phases were washed with water and brine, dried over magnesium sulfate, filtered and reduced under vacuum. The crude product was purified by column chromatography (heptane/ethyl acetate) to give 6.65 g (60% yield) of Intermediate 27-2.

¹H NMR (300 MHz, Methylene Chloride-d2) δ 8.18-8.09 (m, 2H), 7.82 (dd, J=7.7, 1.8 Hz, 1H), 7.52-7.02 (m, 13H), 6.92 (ddd, J=10.8, 8.2, 1.6 Hz, 2H), 6.85-6.75 (m, 4H), 6.62 (t, J=2.1 Hz, 1H), 6.50 (d, J=8.3 Hz, 1H), 2.25-2.04 (m, 12H), 1.42 (d, J=10.6 Hz, 9H).

### Compound 27

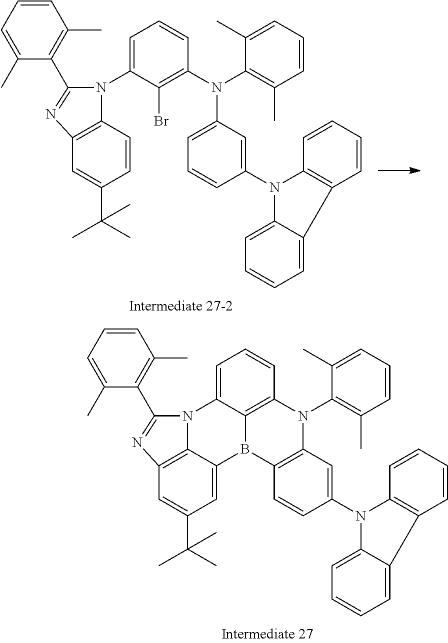 [SMILES: Cc1cccc(C)c1-c1nc2cc(C(C)(C)C)ccc2n1-c1cccc(N(c2cccc(-n3c4ccccc4c4ccccc43)c2)c2c(C)cccc2C)c1Br ; Cc1cccc(C)c1-c1nc2cc(C(C)(C)C)cc3c2n1-c1cccc2c1B3c1ccc(-n3c4ccccc4c4ccccc43)cc1N2c1c(C)cccc1C]

5.85 g (7.37 mmol) of Intermediate 27-2 were dissolved in 100 ml of water-free tert-butylbenzene. 7.76 ml (14.74 mmol) of tert-butyllithium (1.9 M in pentane) were slowly added at −5° C. and it was stirred at this temperature for 45 minutes. The yellowish solution was cooled down to −50° C. and 1.40 ml (14.81 mmol) of tribromoborane were added. The reaction mixture was warmed up to room temperature, stirred for 40 minutes and then cooled down to 0° C. 2.60 ml (14.89 mmol) of N,N-diisopropylethylamine were added and the reaction mixture was heated at 165° C. for 19 hours. The yellow suspension was cooled down and treated with 60 ml of 10% aqueous sodium acetate solution and extracted with 200 ml of ethyl acetate. The organic layer was separated, washed with water, then dried over magnesium sulfate, filtered and concentrated under vacuum. The isolated product was purified by column chromatography (heptane/ethyl acetate). The product was dissolved in 30 ml of dichloromethane and 60 ml of petroleum ether were added. The solution was concentrated under vacuum to a volume of 60 ml and the resulting suspension was filtered, washed with petroleum ether and dried to give 0.421 g (8% yield) of Compound 27.

¹H NMR (300 MHz, Methylene Chloride-d2) δ 9.36 (d, J=8.3 Hz, 1H), 8.96 (d, J=1.7 Hz, 1H), 8.22 (d, J=1.6 Hz, 1H), 8.12 (dt, J=7.5, 1.0 Hz, 2H), 7.66 (dd, J=8.2, 2.0 Hz, 1H), 7.52-7.36 (m, 5H), 7.31 (d, J=3.5 Hz, 5H), 7.29-7.23 (m, 3H), 6.94 (d, J=1.9 Hz, 1H), 6.72 (dd, J=8.2, 0.7 Hz, 1H), 6.47 (dd, J=8.6, 0.7 Hz, 1H), 2.15 (s, 6H), 1.96 (s, 6H), 1.65 (s, 9H).

### Compound 28

### Intermediate 28-1

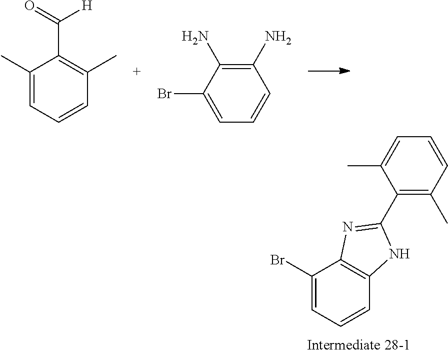

To 20.91 g (112 mmol) of 3-bromobenzene-1,2-diamine in 60 ml of N,N-dimethylacetamide 14.98 g (145 mmol) of sodium bisulfite were added and heated to 100° C. 15.0 g (112 mmol) of 2,6-dimethylbenzaldehyde in 30 ml of N,N-dimethylacetamide were added dropwise at 100° C., then it was stirred at this temperature for 20 hours. The fraction mixture was cooled to room temperature and poured on 300 ml of water and stirred. The suspension was filtered and washed with 250 ml of water. The filter cake was dissolved in 100 ml of ethyl acetate, washed with water and brine, dried over magnesium sulfate, filtered and concentrated under vacuum. The isolated product was purified by column chromatography (heptane/ethyl acetate). The product was suspended in 100 ml of cyclohexane, sonicated in the ultrasonic bath for 2 hours, filtered, washed with cyclohexane and dried to give 21.5 g (64% yield) of Intermediate 28-1.

¹H NMR (300 MHz, DMSO-d₆) δ 12.89 (s, 1H), 7.60 (ddd, J=49.8, 8.0, 0.9 Hz, 1H), 7.43 (ddd, J=7.8, 1.7, 0.9 Hz, 1H), 7.34 (ddd, J=9.3, 6.8, 2.4 Hz, 1H), 7.26-7.08 (m, 3H), 2.10 (d, J=3.1 Hz, 6H).

### Intermediate 28-2

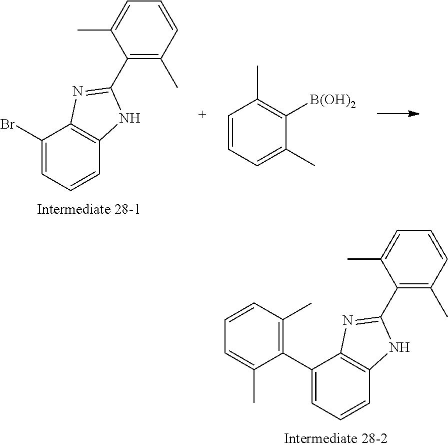 [SMILES: Cc1cccc(C)c1-c1nc2c(Br)cccc2[nH]1 ; Cc1cccc(C)c1B(O)O ; Cc1cccc(C)c1-c1nc2c(-c3c(C)cccc3C)cccc2[nH]1]

9.0 g (29.9 mmol) of Intermediate 28-1, 6.72 g (44.8 mmol) of (2,6-dimethylphenyl)boronic acid and 6.34 g (29.9 mmol) of potassium phosphate were suspended in 93 ml of toluene, 18 ml of ethanol and 38 ml of water. The mixture was evacuated and backfilled with argon 4 times. 1.64 g (4.18 mmol) of DavePhos and 0.469 g (2.09 mmol) of potassium acetate were added and the reaction mixture was heated at 90° C. for 24 hours. The cooled reaction mixture was diluted with 50 ml of ethyl acetate, the phases were separated and the water phase extracted with ethyl acetate. The combined organic phases were washed with water and brine, dried over magnesium sulfate, filtered and concentrated under vacuum. The isolated product was purified by column chromatography (heptane/ethyl acetate) to give 6.28 g (64% yield) of Intermediate 28-2.

LC-MS: 325.1 [M−H]⁻

### Intermediate 28-3

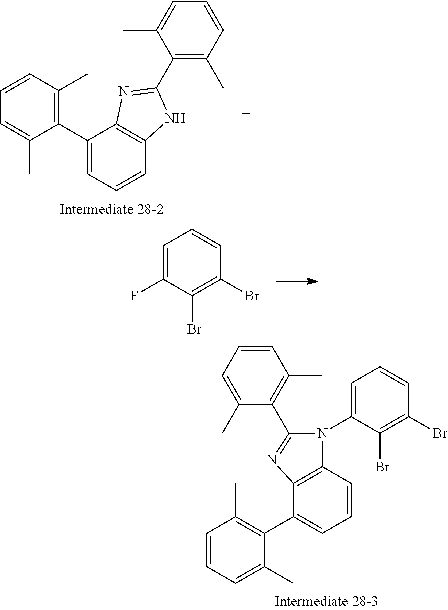 [SMILES: Cc1cccc(C)c1-c1nc2c(-c3c(C)cccc3C)cccc2[nH]1 ; Fc1cccc(Br)c1Br ; Cc1cccc(C)c1-c1cccc2c1nc(-c1c(C)cccc1C)n2-c1cccc(Br)c1Br]

5.0 g (15.32 mmol) of Intermediate 28-2 and 8.56 g (33.7 mmol) of 1,2-dibromo-3-fluorobenzene were dissolved in 80 ml of N,N-dimethylformamide. 16.26 g (77 mmol) of potassium phosphate tribasic were added. The reaction mixture was heated at 130° C. (outside temperature) for 24 hours, then cooled to room temperature and filtered. The solvent of the filtrate was removed at reduced pressure and the isolated solid was dissolved in 50 ml of ethyl acetate, then 50 ml of water were added and the phases were separated. The water phase was extracted with ethyl acetate, the combined organic phases were washed with water and brine, dried over magnesium sulfate, filtered and concentrated under vacuum. The isolated product was purified by column chromatography (heptane/ethyl acetate) to give 6.7 g (78% yield) of Intermediate 28-3.

¹H NMR (300 MHz, Methylene Chloride-d₂) δ 7.69 (dd, J=7.6, 2.0 Hz, 1H), 7.42-7.30 (m, 1H), 7.20-7.08 (m, 8H), 6.98 (dd, J=33.5, 7.6 Hz, 2H), 2.24-1.99 (m, 12H).

### Intermediate 28-4

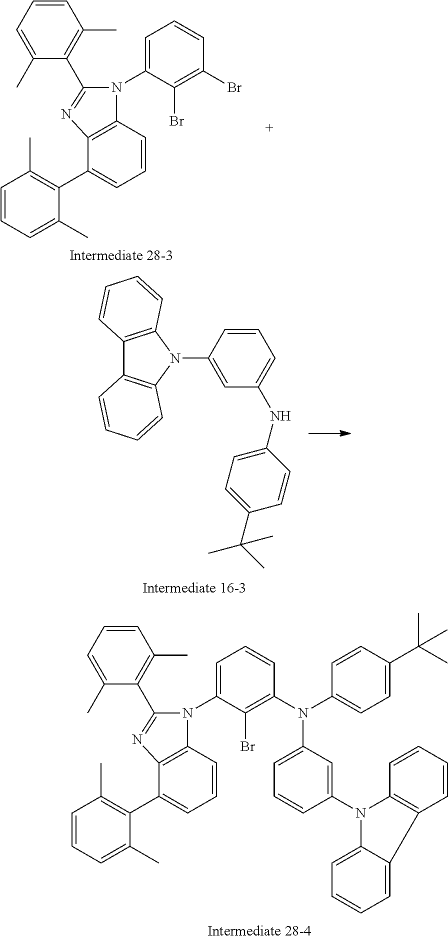 [SMILES: Cc1cccc(C)c1-c1cccc2c1nc(-c1c(C)cccc1C)n2-c1cccc(Br)c1Br ; CC(C)(C)c1ccc(Nc2cccc(-n3c4ccccc4c4ccccc43)c2)cc1 ; Cc1cccc(C)c1-c1cccc2c1nc(-c1c(C)cccc1C)n2-c1cccc(N(c2ccc(C(C)(C)C)cc2)c2cccc(-n3c4ccccc4c4ccccc43)c2)c1Br]

9.4 g (16.78 mmol) of Intermediate 28-3, 7.21 g (18.45 mmol) of Intermediate 16-3 and 4.03 g (41.9 mmol) of sodium tert-butoxide were suspended in 100 ml of toluene. The mixture was evacuated and backfilled with argon 4 times. 0.779 g (2.68 mmol) of tri-tert-butylphosphonium tetrafluoroborate and 0.614 g (0.671 mmol) of Pd₂(dba)₃ were added and the reaction mixture was heated at 80° C. for 5 hours, then cooled to room temperature. 60 ml of water were added and stirred. The phases were separated, the water phase was extracted with ethyl acetate, the combined organic phases were washed with water and brine, dried over magnesium sulfate, filtered and reduced under vacuum. The crude product was purified by column chromatography (heptane/ethyl acetate) to give 12.73 g (87% yield) of Intermediate 28-4.

¹H NMR (300 MHz, Methylene Chloride-d₂) δ 8.13 (dt, J=7.7, 1.0 Hz, 2H), 7.48-6.91 (m, 26H), 2.14 (s, 3H), 2.08 (s, 3H), 2.04 (s, 3H), 1.90 (s, 3H), 1.31 (s, 9H).

### Compound 28

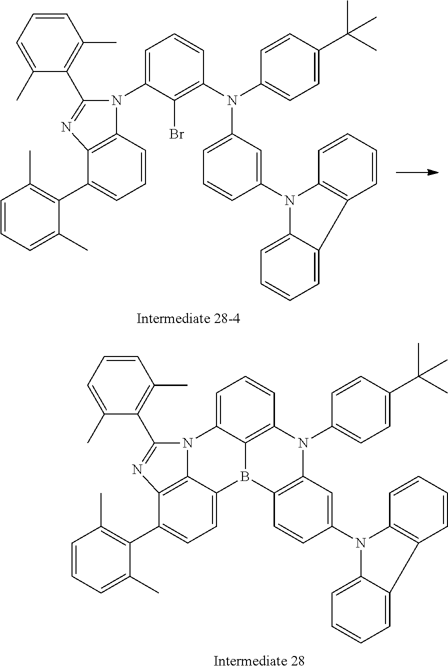 [SMILES: Cc1cccc(C)c1-c1cccc2c1nc(-c1c(C)cccc1C)n2-c1cccc(N(c2ccc(C(C)(C)C)cc2)c2cccc(-n3c4ccccc4c4ccccc43)c2)c1Br ; Cc1cccc(C)c1-c1ccc2c3c1nc(-c1c(C)cccc1C)n3-c1cccc3c1B2c1ccc(-n2c4ccccc4c4ccccc42)cc1N3c1ccc(C(C)(C)C)cc1]

13.86 g (15.93 mmol) of Intermediate 28-4 were dissolved in 250 ml of water-free tert-butylbenzene. 18.74 ml (31.9 mmol) of tert-butyllithium (1.9 M in pentane) were slowly added at 0° C. and it was stirred at this temperature for 50 minutes. The yellowish solution was cooled down to −50° C. and 6.02 ml (63.7 mmol) of tribromoborane were added. The reaction mixture was warmed up to room temperature, stirred for 1 hour and then cooled down to 0° C. 22.26 ml (127 mmol) of N,N-diisopropylethylamine were added and the reaction mixture was heated at 165° C. for 18 hours. 27.8 ml (159 mmol) of N,N-diisopropylethylamine were added again and the reaction mixture was heated at 165° C. for 6 hours. The yellow suspension was cooled down and treated with 120 ml of 10% aqueous sodium acetate solution and extracted with 400 ml of ethyl acetate. The organic layer was separated, washed with water, dried over magnesium sulfate, filtered and concentrated under vacuum. The isolated product was purified by column chromatography (dichloromethane) to give 294 mg (2.7% yield) of Compound 28.

¹H NMR (300 MHz, Methylene Chloride-od) δ 9.35 (d, J=8.3 Hz, 1H), 8.91 (d, J=7.6 Hz, 1H), 8.11 (dt, J=7.7, 1.1 Hz, 2H), 7.72-7.67 (m, 2H), 7.62 (dd, J=8.2, 1.9 Hz, 1H), 7.55-7.50 (m, 3H), 7.43-7.35 (m, 5H), 7.30-7.18 (m, 8H), 7.03 (d, J=1.9 Hz, 1H), 6.79-6.74 (m, 1H), 6.65-6.58 (m, 1H), 2.17 (s, 6H), 2.12 (s, 6H), 1.34 (s, 9H).

### Compound 29

### Intermediate 29-1

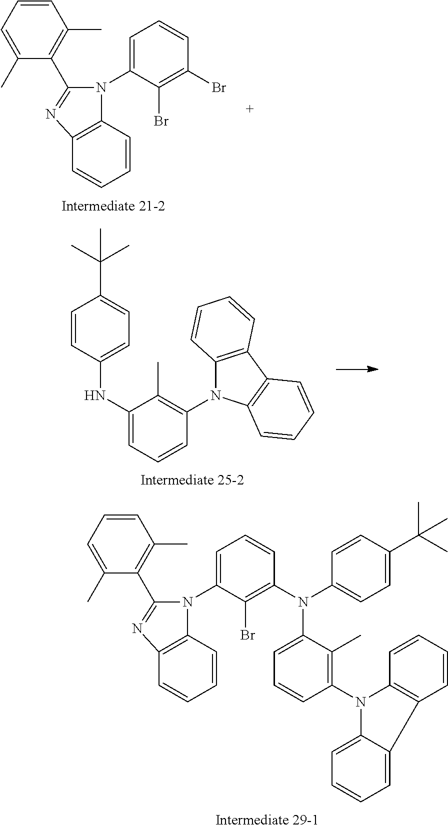

6.50 g (14.25 mmol) of Intermediate 21-2, 5.76 g (14.25 mmol) of Intermediate 25-2 and 3.42 g (35.6 mmol) of sodium tert-butoxide were suspended in 200 ml of o-xylene. The mixture was evacuated and backfilled with argon 4 times. 0.331 g (1.140 mmol) of tri-tert-butylphosphonium tetrafluoroborate and 0.266 g (0.285 mmol) of Pd₂(dba)₃ were added and the reaction mixture was heated at 115° C. for 2 hours, then cooled to room temperature. 200 ml of water were added and stirred. The phases were separated, the water phase was extracted with ethylaceteate, the combined organic phases were washed with water and brine, dried over magnesium sulfate, filtered and reduced under vacuum. The crude product was purified by column chromatography (heptane/ethyl acetate) to give 7.1 g (64% yield) of Intermediate 29-1.

¹H NMR (300 MHz, Methylene Chloride-d₂) δ 8.18-8.10 (m, 2H), 7.86-7.80 (m, 1H), 7.40-7.17 (m, 13H), 7.13-6.92 (m, 7H), 6.74 (d, J=8.2 Hz, 2H), 2.20 (s, 3H), 2.03 (s, 3H), 1.57 (s, 3H), 1.31 (s, 9H).

### Compound 29

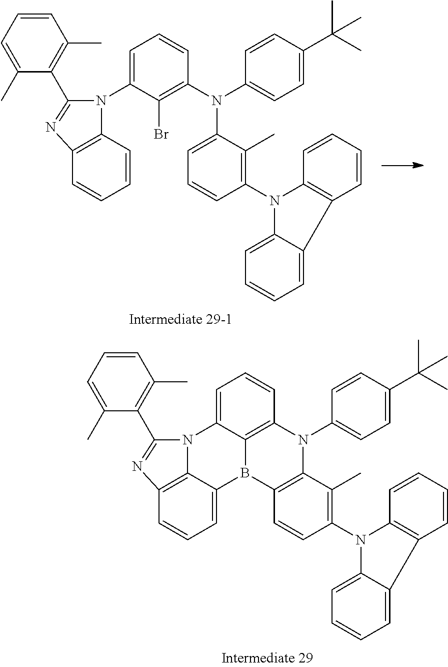 [SMILES: Cc1cccc(C)c1-c1nc2ccccc2n1-c1cccc(N(c2ccc(C(C)(C)C)cc2)c2cccc(-n3c4ccccc4c4ccccc43)c2C)c1Br ; Cc1cccc(C)c1-c1nc2cccc3c2n1-c1cccc2c1B3c1ccc(-n3c4ccccc4c4ccccc43)c(C)c1N2c1ccc(C(C)(C)C)cc1]

1.028 g (1.318 mmol) of Intermediate 29-1 were dissolved in 20 ml of water-free tert-butylbenzene. 0.70 ml (1.318 mmol) of tert-butyllithium (1.9M in pentane) were slowly added at −20° C., then it was heated up to room temperature and stirred for 30 minutes. The yellowish solution was cooled down to −35° C. and 0.50 ml (5.21 mmol) of tribromoborane were added. The reaction mixture was warmed up to room temperature, stirred for 1 hour and then cooled down to 0° C. 0.75 ml (5.34 mmol) of N,N-diisopropylethylamine were added and the reaction mixture was heated at 160° C. for 14 hours. The yellow suspension was cooled to room temperature, treated with 20 ml of 10% aqueous sodium acetate solution and extracted with 60 ml of ethyl acetate. The organic layer was separated, washed with water, dried over magnesium sulfate, filtered and concentrated under vacuum. The isolated product was purified by column chromatography (heptane/ethyl acetate) to give 0.08 g (8.5% yield) of Compound 29.

LC-MS: 707.3 [M−H]⁻

### Compound 30

### Intermediate 30-1

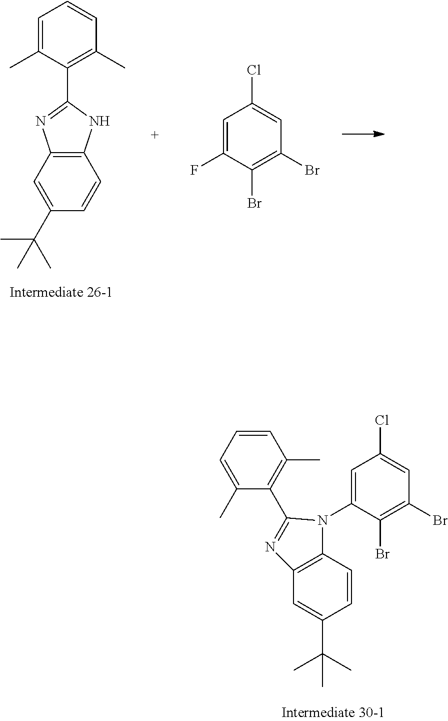 [SMILES: Cc1cccc(C)c1-c1nc2cc(C(C)(C)C)ccc2[nH]1 ; Fc1cc(Cl)cc(Br)c1Br ; Cc1cccc(C)c1-c1nc2cc(C(C)(C)C)ccc2n1-c1cc(Cl)cc(Br)c1Br]

30.0 g (108 mmol) of Intermediate 26-1, 34.2 g (119 mmol) of 1,2-dibromo-5-chloro-3-fluorobenzene and 91 g (0.43 mol) of potassium phosphate were suspended in 275 ml of N,N-dimethylformamide, followed by heating at 80° C. during 4 hours. The reaction mixture was cooled down to room temperature and poured into 600 ml of water. The resulting suspension was filtered and the solid washed with 400 ml of water. The solid was stirred in 300 ml of acetone during 30 minutes, then filtered and washed with 200 ml of acetone, giving 60.1 g of a light grey solid. The solid was purified by MPLC in three portions with the CombiFlash Companion (silica gel, dichloromethane/heptane). The product fractions were combined and concentrated under vacuum to give 26.4 g (45% yield) of Intermediate 30-1 as a white solid.

¹H NMR (300 MHz, CD₂Cl₂) δ 7.93 (dd, 1H), 7.73 (d, 1H), 7.46 (dd, 1H), 7.27 (t, 1H), 7.21-6.99 (m, 4H), 2.27 (s, 3H), 2.13 (s, 3H), 1.48 (s, 9H).

### Intermediate 30-2

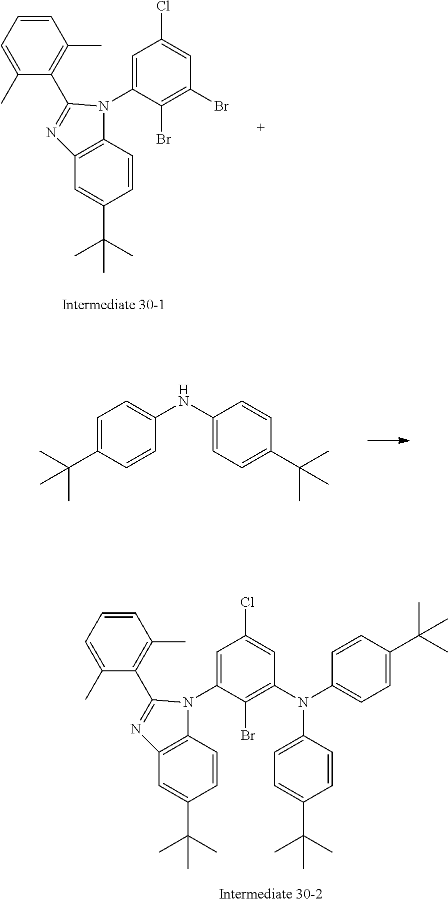 [SMILES: Cc1cccc(C)c1-c1nc2cc(C(C)(C)C)ccc2n1-c1cc(Cl)cc(Br)c1Br ; CC(C)(C)c1ccc(Nc2ccc(C(C)(C)C)cc2)cc1 ; Cc1cccc(C)c1-c1nc2cc(C(C)(C)C)ccc2n1-c1cc(Cl)cc(N(c2ccc(C(C)(C)C)cc2)c2ccc(C(C)(C)C)cc2)c1Br]

26.0 g (47.6 mmol) of Intermediate 30-1, 16.7 g (59.4 mmol) of bis(4-(tert-butyl)phenyl)amine, 871 mg (0.95 mmol) of tris(dibenzylideneacetone)dipalladium(0), 1.10 g (3.80 mmol) of tri-tert-butylphosphonium tetrafluoroborate, and 11.4 g (119 mmol) of sodium tert-butoxide were suspended in 300 ml of toluene. The dark suspension was 3 times evacuated and backfilled with argon and heated at 72° C. during 21 hours. 100 ml of water and 1.0 g of sodium cyanide were added and the reaction mixture stirred during 30 minutes without heating. The reaction mixture was extracted with water (2×100 ml), dried over sodium sulfate and concentrated under vacuum. The solid was recrystallized from 100 ml of acetonitrile and washed with 30 ml of cold acetonitrile to give 30.5 g (86% yield) of Intermediate 30-2.

¹H NMR (300 MHz, CD₂Cl₂) δ 7.92 (s, 1H), 7.46 (dd, 1H), 7.39-7.23 (m, 5H), 7.24-7.05 (m, 4H), 7.01 (d, 1H), 6.88-6.74 (m, 4H), 2.23 (s, 3H), 2.11 (s, 3H), 1.47 (s, 9H), 1.36 (s, 18H).

### Intermediate 30-3

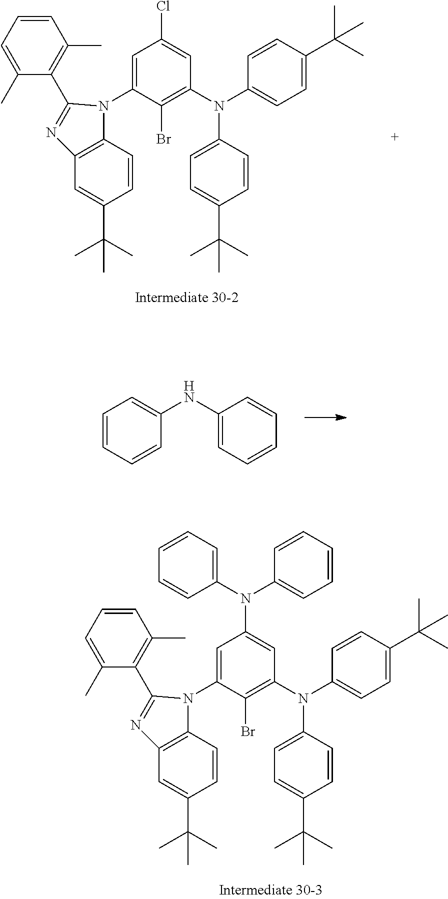 [SMILES: Cc1cccc(C)c1-c1nc2cc(C(C)(C)C)ccc2n1-c1cc(Cl)cc(N(c2ccc(C(C)(C)C)cc2)c2ccc(C(C)(C)C)cc2)c1Br ; c1ccc(Nc2ccccc2)cc1 ; Cc1cccc(C)c1-c1nc2cc(C(C)(C)C)ccc2n1-c1cc(N(c2ccccc2)c2ccccc2)cc(N(c2ccc(C(C)(C)C)cc2)c2ccc(C(C)(C)C)cc2)c1Br]

10.0 g (13.4 mmol) of Intermediate 30-2, 3.4 g (20.0 mmol) of diphenylamine, 245 mg (0.27 mmol) of tris(dibenzylideneacetone)dipalladium(0), 311 mg (1.07 mmol) of tri-tert-butylphosphonium tetrafluoroborate, and 3.22 g (33.5 mmol) of sodium tert-butoxide were suspended in 150 ml of toluene. The dark suspension was 3 times evacuated and backfilled with argon and heated at 108° C. during 23 hours. 245 mg (0.27 mmol) of tris(dibenzylidene-acetone)dipalladium(0) and 311 mg (1.07 mmol) of tri-tert-butylphosphonium tetrafluoroborate were added and heating continued at 108° C. during 24 hours. The dark suspension was cooled down, followed by the addition of 100 ml of water and 0.5 g of sodium cyanide. The suspension was stirred during 30 minutes and extracted with water (2×100 ml). The organic layer was separated, then dried over sodium sulfate, and concentrated under vacuum. The dark resin was purified by MPLC with the CombiFlash Companion (silica gel, heptane/0-15% gradient of ethyl acetate) to give 8.1 g (69% yield) of Intermediate 30-3.

¹H NMR (300 MHz, CD₂Cl₂) δ 7.85 (s, 1H), 7.44 (d, 1H), 7.37 (d, 1H), 7.33-7.17 (m, 9H), 7.17-7.03 (m, 4H), 6.94-6.80 (m, 9H), 6.44 (d, 1H), 2.08 (s, 3H), 1.77 (s, 3H), 1.45 (s, 9H), 1.36 (s, 18H).

### Compound 30

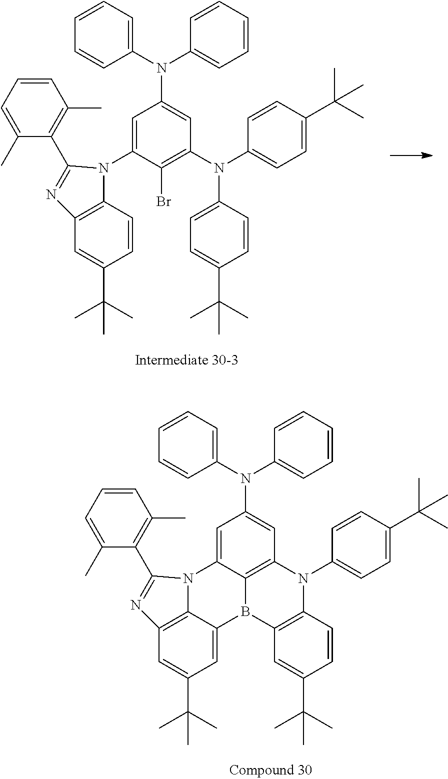 [SMILES: Cc1cccc(C)c1-c1nc2cc(C(C)(C)C)ccc2n1-c1cc(N(c2ccccc2)c2ccccc2)cc(N(c2ccc(C(C)(C)C)cc2)c2ccc(C(C)(C)C)cc2)c1Br ; Cc1cccc(C)c1-c1nc2cc(C(C)(C)C)cc3c2n1-c1cc(N(c2ccccc2)c2ccccc2)cc2c1B3c1cc(C(C)(C)C)ccc1N2c1ccc(C(C)(C)C)cc1]

8.00 g (9.09 mmol) of Intermediate 30-3 were dissolved in 111 ml of water-free tert-butylbenzene. 9.57 ml of tert-butyllithium (1.9 M in pentane) were slowly added at −24° C. and stirred at −3° C. during 20 minutes. The orange solution was cooled down to −54° C. and 1.72 ml (18.2 mmol) of tribromoborane were added. The brown suspension was warmed up to 6° C. during 15 minutes and cooled down to −1° C. 3.18 ml (18.2 mmol) of N,N-diisopropylethyl-amine were slowly added and the reaction mixture heated up to 132° C. during 1 hour. The temperature was increased to 152° C. and 3.18 ml (18.2 mmol) of N,N-diisopropylethyl-amine were slowly added. Heating was continued at 153° C. and the same amount (3.18 ml) of N,N-diisopropylethylamine was added twice each time after a period of additional 2 hours reaction time. Heating was continued for 17 hours. The orange suspension was cooled down and treated with 50 ml of 10% aqueous sodium acetate solution and 300 ml of heptane. The suspension was stirred at room temperature during 1 hour, then filtered and the solid washed with 50 ml of heptane. The isolated product was further purified by MPLC with the CombiFlash Companion (silica gel, dichloromethane/0-10% gradient of ethyl acetate) and the product fractions diluted with 50 ml of heptane and concentrated under vacuum until a suspension formed. The suspension was filtered and the solid washed with heptane. The MPLC purification was repeated to give 1.01 g (14% yield) of Compound 30 as a yellow solid.

¹H NMR (300 MHz, CD₂Cl₂) δ 9.13 (d, 1H), 8.95 (d, 1H), 8.14 (d, 1H), 7.58 (dd, 1H), 7.54-7.48 (m, 2H), 7.28-7.10 (m, 6H), 7.09-6.93 (m, 3H), 6.92-6.73 (m, 7H), 6.36 (d, 1H), 5.95 (d, 1H), 2.02 (s, 6H), 1.66 (s, 9H), 1.54 (s, 9H), 1.37 (s, 9H).

### Compound 31

### Intermediate 31-1

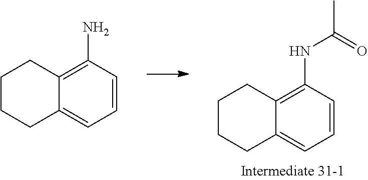

132 g (0.90 mol) of 5,6,7,8-tetrahydro-1-naphthylamine were dissolved in 750 ml of dichloromethane. 110 g (1.08 mol) of acetic anhydride were dropwise added during 15 minutes at a maximum temperature of 30° C., and stirring continued during 1 hour. The reaction mixture was poured into 1400 ml of 10% aqueous sodium carbonate solution, followed by the addition of 700 ml of dichloromethane. The mixture was stirred during 10 minutes. The aqueous layer was separated and washed with 300 ml of dichloromethane. The combined organic layers were washed with 1000 ml of water, dried over magnesium sulfate and concentrated under vacuum. The solid was stirred in 1000 ml of heptane and stirred during 10 minutes. The suspension was filtered and the solid washed with 300 ml of heptane to give 161 g of Intermediate 31-1 as a solid. The filtrate was concentrated under vacuum to a volume of 400 ml. The suspension was filtered, the solid washed with heptane (3×80 ml) to give another 8.97 g of Intermediate 31-1 for a total yield of 99%.

¹H NMR (400 MHz, CDCl₃) δ 7.55 (d, 1H), 7.20-6.81 (m, 3H), 2.79 (t, 2H), 2.60 (t, 2H), 2.19 (s, 3H), 1.90-1.66 (m, 4H).

### Intermediate 31-2

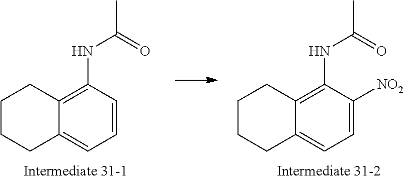 [SMILES: CC(=O)Nc1cccc2c1CCCC2 ; CC(=O)Nc1c([N+](=O)[O-])ccc2c1CCCC2]

165 g (0.87 mol) of Intermediate 31-1 were suspended in 550 ml of acetic acid and 500 ml of acetic anhydride. 82 ml (1.18 mol) of 65% nitric acid were dropwise added during 25 minutes at a maximum temperature range of 30° C. by cooling with an ice-bath. The orange-brown solution was stirred at room temperature during 2 hours. The suspension was cooled down to 0° C., filtered, and the solid washed twice with acetic acid (2×80 ml) and heptane (3×130 ml) to give 106 g (52% yield) of Intermediate 31-2 as a white solid.

¹H NMR (400 MHz, CDCl₃) δ 8.27 (s, 1H), 7.74 (d, 1H), 7.10 (d, 1H), 2.86 (t, 2H), 2.67 (t, 2H), 2.21 (s, 3H), 1.88-1.69 (m, 4H).

### Intermediate 31-3

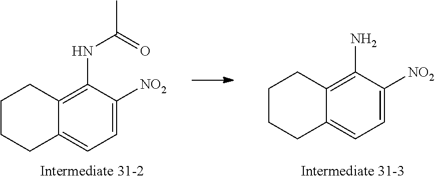 [SMILES: CC(=O)Nc1c([N+](=O)[O-])ccc2c1CCCC2 ; Nc1c([N+](=O)[O-])ccc2c1CCCC2]

141 g (0.60 mol) of Intermediate 31-2 were suspended in 900 ml of ethanol and slowly treated with 1200 ml of concentrated hydrogen chloride solution. The orange solution was stirred at room temperature during 14 hours. The reaction mixture was poured into two liters of an ice-water mixture, followed by careful addition of 580 g of sodium carbonate. The orange suspension was diluted with two liters of water and cooled with 1 kg ice to 10° C. and stirred during 10 minutes. The suspension was filtered and the solid washed with water (4×400 ml) to give 114 g (99% yield) of Intermediate 31-3.

¹H NMR (400 MHz, CDCl₃) δ 7.93 (d, 1H), 6.46 (d, 1H), 6.28 (br. s, 2H), 2.75 (t, 2H), 2.46 (t, 2H), 1.97-1.85 (m, 2H), 1.83-1.73 (m, 2H).

### Intermediate 31-4

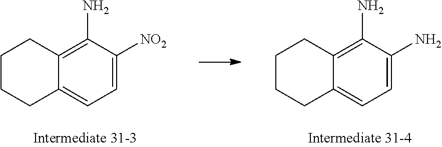 [SMILES: Nc1c([N+](=O)[O-])ccc2c1CCCC2 ; Nc1ccc2c(c1N)CCCC2]

114 g (0.60 mol) of Intermediate 31-3 and 59.4 g (0.30 mol) of sodium hydroxide in 400 ml of ethanol were heated up to reflux temperature. The oil bath was removed and 155 g (2.4 mol) of zinc dust were added in 5 g portions over a period of 45 minutes, and stirring continued during 1 hour. The reaction mixture was cooled down to 40° C., then filtered, and the solid washed with ethanol (3×100 ml). The solid was dissolved in 600 ml of ethyl acetate and the solution treated with 25 g of activated charcoal, followed by heating under reflux during 20 minutes. The mixture was filtered, and the solid residue washed with hot ethyl acetate (4×125 ml). The filtrate was concentrated under vacuum to give 94.5 g (98%) of Intermediate 31-4 as a brown solid.

¹H NMR (400 MHz, CDCl₃) δ 6.60 (d, 1H), 6.52 (d, 1H), 3.46 (s, 2H), 3.20 (s, 2H), 2.74 (t, 2H), 2.54 (t, 2H), 1.95-1.84 (m, 2H), 1.83-1.71 (m, 2H).

### Intermediate 31-5

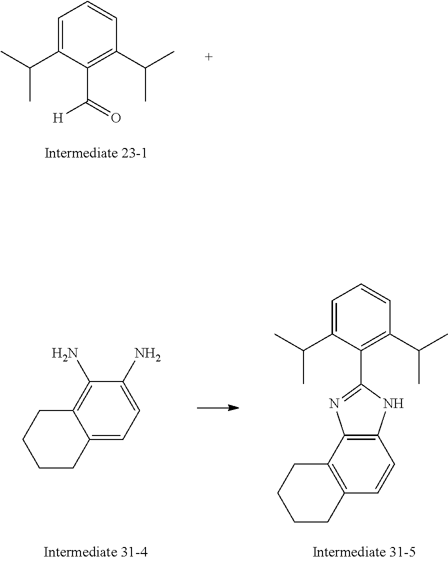 [SMILES: CC(C)c1cccc(C(C)C)c1C=O ; Nc1ccc2c(c1N)CCCC2 ; CC(C)c1cccc(C(C)C)c1-c1nc2c3c(ccc2[nH]1)CCCC3]

10.2 g (63.1 mmol) of Intermediate 31-4 and 6.56 g (63.1 mmol) of sodium bisulfite in 60 ml of N,N-dimethylacetamide were heated to 92° C. 12.0 g (63.1 mmol) of Intermediate 23-1 in 20 ml of N,N-dimethylacetamide were dropwise added during 15 minutes, and heating continued at 98° C. during 3 hours. The reaction mixture was poured into 150 ml of water, and the resulting yellow suspension filtered and the solid washed with 100 ml of water. The solid was suspended in 100 ml of heptane and washed with 100 ml of heptane to give 19.1 g (91% yield) of Intermediate 31-5 as a solid.

ESI-MS (positive, m/z): exact mass of C₂₃H₂₈N₂=332.23; found 333.2 [M+1]⁺.

### Intermediate 31-6

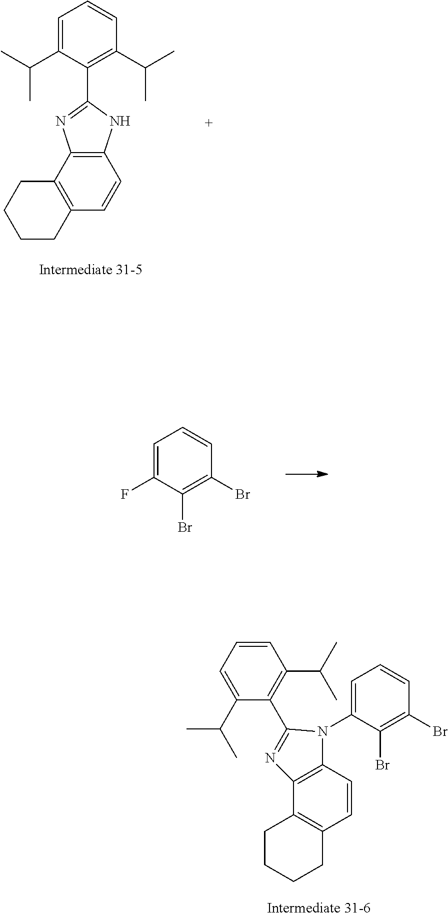 [SMILES: CC(C)c1cccc(C(C)C)c1-c1nc2c3c(ccc2[nH]1)CCCC3 ; Fc1cccc(Br)c1Br ; CC(C)c1cccc(C(C)C)c1-c1nc2c3c(ccc2n1-c1cccc(Br)c1Br)CCCC3]

19.0 g (57.1 mmol) of Intermediate 31-5, 16.0 g (62.9 mmol) of 2,3-dibromofluorobenzene, and 48.5 g (229 mmol) of potassium phosphate were suspended in 200 ml of N,N-dimethyl-formamide, followed by heating at 138° C. during 5 hours. The reaction mixture was cooled down to room temperature and poured into 800 ml of water. The resulting suspension was filtered and the solid washed with water. The solid was suspended in 300 ml of acetone, the suspension filtered, and the solid washed with 100 ml of acetone to give 28.1 g (87% yield) of Intermediate 31-6.

¹H NMR (400 MHz, CD₂Cl₂) δ 7.69 (dd, 1H), 7.40 (m, 1H), 7.33-7.20 (dd, 1H), 7.18-6.99 (m, 4H), 6.92 (d, 1H), 3.24 (m, 2H), 3.04-2.87 (m, 2H), 2.82-2.66 (m, 1H), 2.58-2.43 (m, 1H), 2.09-1.87 (m, 4H), 1.34-1.26 (2 d, 6H), 1.10 (d, 3H), 0.85 (d, 3H).

### Intermediate 31-7

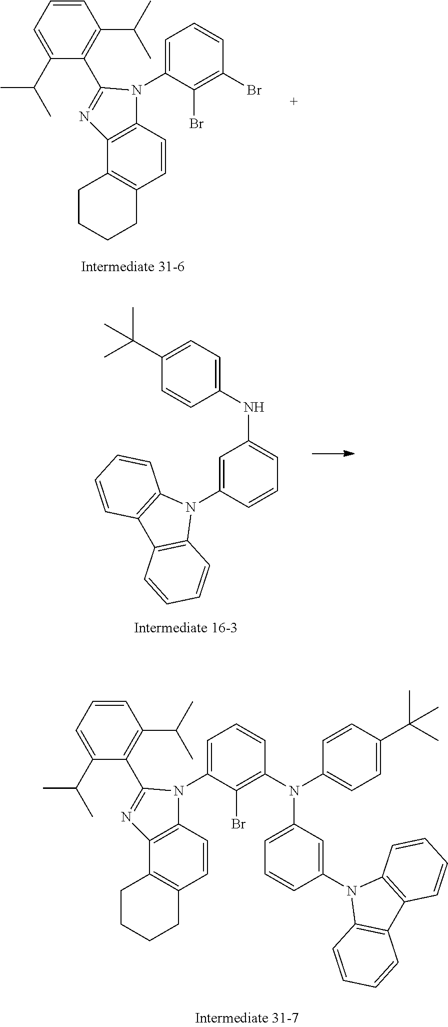 [SMILES: CC(C)c1cccc(C(C)C)c1-c1nc2c3c(ccc2n1-c1cccc(Br)c1Br)CCCC3 ; CC(C)(C)c1ccc(Nc2cccc(-n3c4ccccc4c4ccccc43)c2)cc1 ; CC(C)c1cccc(C(C)C)c1-c1nc2c3c(ccc2n1-c1cccc(N(c2ccc(C(C)(C)C)cc2)c2cccc(-n4c5ccccc5c5ccccc54)c2)c1Br)CCCC3]

14.0 g (24.7 mmol) of Intermediate 31-6, 9.65 g (24.7 mmol) of Intermediate 16-3, 453 mg (0.49 mmol) of tris(dibenzylideneacetone)dipalladium(0), 574 mg (1.98 mmol) of tri-tert-butylphosphonium tetrafluoroborate, and 5.94 g (61.8 mmol) of sodium tert-butoxide were suspended in 200 ml of o-xylene. The dark suspension was 3 times evacuated and backfilled with argon and heated at 122° C. during 5 hours. The reaction mixture was cooled down and filtered through a 3 cm layer of silica gel followed by rinsing the silica gel layer with 100 ml of toluene. The collected eluents were concentrated under vacuum and the product purified by MPLC with the CombiFlash Companion (silica gel, dichloromethane). The isolated product was further purified by MPLC with the CombiFlash Companion (silica gel, heptane/0-10% gradient of ethyl acetate) to give 14.4 g (67% yield) of Intermediate 31-7.

ESI-MS (positive, m/z): exact mass of C₅₇H₅₅BrN₄=874.36; found 875.6 [M+1]⁺.

### Compound 31

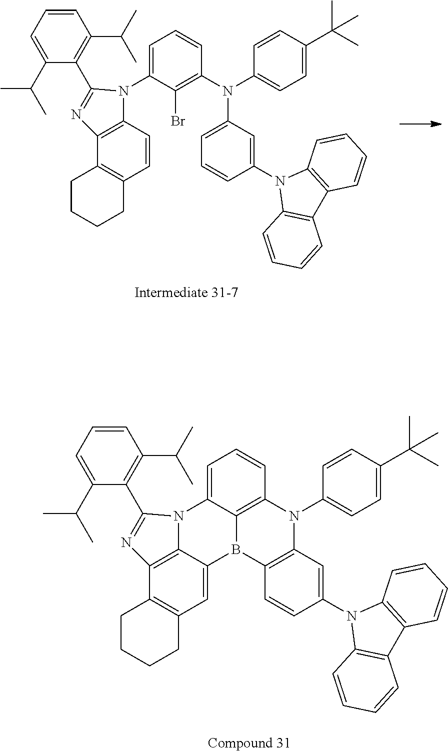 [SMILES: CC(C)c1cccc(C(C)C)c1-c1nc2c3c(ccc2n1-c1cccc(N(c2ccc(C(C)(C)C)cc2)c2cccc(-n4c5ccccc5c5ccccc54)c2)c1Br)CCCC3 ; CC(C)c1cccc(C(C)C)c1-c1nc2c3c(cc4c2n1-c1cccc2c1B4c1ccc(-n4c5ccccc5c5ccccc54)cc1N2c1ccc(C(C)(C)C)cc1)CCCC3]

8.00 g (9.13 mmol) of Intermediate 31-7 were dissolved in 120 ml of water-free tert-butylbenzene. 9.61 ml of tert-butyllithium (1.9 M in pentane) were slowly added at −4° C. and stirred at 3° C. during 15 minutes. The dark solution was cooled down to −52° C. and 1.73 ml (18.2 mmol) of tribromoborane were added. The brown suspension was warmed up to 6° C. during 15 minutes and cooled down to −2° C. 3.19 ml (18.3 mmol) of N,N-diisopropylethyl-amine were slowly added and the reaction mixture heated at 123° C. during 2 hours first, followed by heating at 148° C. during 1 hour. The orange suspension was cooled down and treated with 50 ml of 10% aqueous sodium acetate solution and 200 ml of heptane. The mixture was filtered, the organic phase separated and extracted with water (2×100 ml), dried over sodium sulfate and concentrated under vacuum. The yellow resin was further purified by MPLC with the CombiFlash Companion (silica gel, dichloromethane), and the collected product fractions combined and concentrated under vacuum. The resulting solid was dissolved in 50 ml of dichloromethane and 50 ml of ethyl acetate and concentrated under vacuum to a volume of 40 ml until a suspension formed. The suspension was filtered and washed with ethyl acetate to give 1.04 g (14% yield) of Compound 31 as a yellow solid.

¹H NMR (300 MHz, CD₂Cl₂) δ 9.35 (d, 1H), 8.64 (s, 1H), 8.15 (d, 2H), 7.77-7.60 (m, 4H), 7.55 (d, 2H), 7.50-7.36 (m, 6H), 7.35-7.21 (m, 3H), 7.04 (d, 1H), 6.79 (d, 1H), 6.63 (s, 1H), 3.47 (br. s, 2H), 3.26 (br. s, 2H), 2.72-2.50 (m, 2H), 2.18-1.99 (m, 4H), 1.37 (s, 9H), 1.24 (d, 6H), 1.04 (d, 6H).

### Compound 32

### Intermediate 32-1

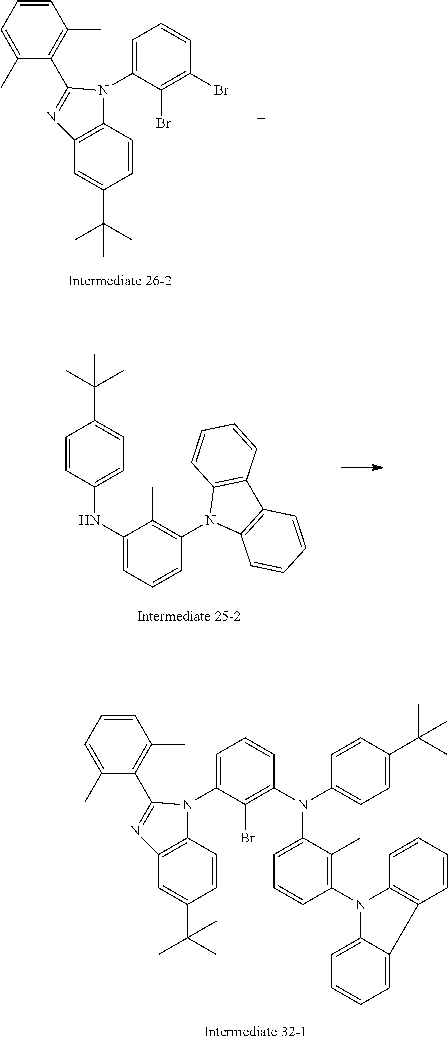 [SMILES: Cc1cccc(C)c1-c1nc2cc(C(C)(C)C)ccc2n1-c1cccc(Br)c1Br ; Cc1c(Nc2ccc(C(C)(C)C)cc2)cccc1-n1c2ccccc2c2ccccc21 ; Cc1cccc(C)c1-c1nc2cc(C(C)(C)C)ccc2n1-c1cccc(N(c2ccc(C(C)(C)C)cc2)c2cccc(-n3c4ccccc4c4ccccc43)c2C)c1Br]

2.13 g (4.16 mmol) of Intermediate 26-2, 1.766 g (4.37 mmol) of Intermediate 25-2 and 0.799 g (8.23 mmol) of sodium tert-butoxide were suspended in 50 ml of o-xylene. The mixture was evacuated and backfilled with argon 4 times. 0.097 g (0.333 mmol) of tri-tert-butylphosphonium tetrafluoroborate and 0.076 g (0.083 mmol) of Pd₂(dba)₃ were added and the reaction mixture was heated at 115° C. for 1 hour, then cooled to room temperature. 50 ml of water were added and stirred. The phases were separated, the water phase was extracted with ethyl acetate, the combined organic phases were washed with water and brine, dried over magnesium sulfate, filtered and reduced under vacuum. The crude product was purified by column chromatography (heptane/dichloromethane) to give 2.17 g (59% yield) of Intermediate 32-1.

¹H NMR (300 MHz, Methylene Chloride-d₂) δ 8.17-8.12 (m, 2H), 7.86 (dd, J=1.7, 0.6 Hz, 1H), 7.40-7.16 (m, 12H), 7.10-6.92 (m, 7H), 6.74 (d, J=8.2 Hz, 2H), 2.20 (s, 3H), 2.03 (s, 3H), 1.57 (s, 3H), 1.42 (s, 9H), 1.31 (s, 9H).

### Compound 32

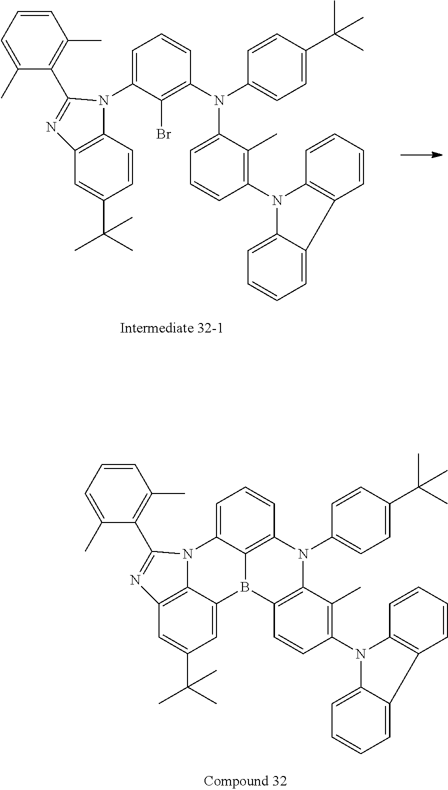 [SMILES: Cc1cccc(C)c1-c1nc2cc(C(C)(C)C)ccc2n1-c1cccc(N(c2ccc(C(C)(C)C)cc2)c2cccc(-n3c4ccccc4c4ccccc43)c2C)c1Br ; Cc1cccc(C)c1-c1nc2cc(C(C)(C)C)cc3c2n1-c1cccc2c1B3c1ccc(-n3c4ccccc4c4ccccc43)c(C)c1N2c1ccc(C(C)(C)C)cc1]

2.16 g (2.58 mmol) of Intermediate 32-1 were dissolved in 40 ml of water-free tert-butylbenzene. 2.72 ml (5.17 mmol) of tert-butyllithium (1.9M in pentane) were slowly added at 0° C. and it was stirred at this temperature for 50 minutes. The yellowish solution was cooled down to −50° C. and 0.489 ml (5.17 mmol) of tribromoborane were added. The reaction mixture was warmed up to room temperature, stirred for 1 hour and then cooled down to 0° C. 0.903 ml (5.17 mmol) of N,N-diisopropylethylamine were added and the reaction mixture was heated at 165° C. for 20 hours. 0.90 ml (5.17 mmol) of N,N-diisopropylethylamine were added again and the reaction mixture was heated at 165° C. for 23 hours. The yellow suspension was cooled down and treated with 40 ml of 10% aqueous sodium acetate solution and extracted with 200 ml of ethyl acetate. The organic layer was separated, washed with water, dried over magnesium sulfate, filtered and concentrated under vacuum. The isolated product was purified by column chromatography (dichloromethane) to give 0.292 g (14.7% yield) of Compound 32.

¹H NMR (300 MHz, Methylene Chloride-d2) δ 9.20 (d, J=2.5 Hz, 1H), 8.97 (d, J=1.7 Hz, 1H), 8.23-8.13 (m, 3H), 7.83-7.68 (m, 3H), 7.47 (dddd, J=16.6, 15.6, 7.9, 1.5 Hz, 3H), 7.38-7.10 (m, 8H), 6.90 (d, J=9.0 Hz, 1H), 6.68 (d, J=8.1 Hz, 1H), 6.61 (d, J=8.4 Hz, 1H), 2.11 (d, J=6.4 Hz, 6H), 1.62 (s, 9H), 1.54 (s, 9H), 1.49 (s, 3H).

### Compound 33

### Intermediate 33-1

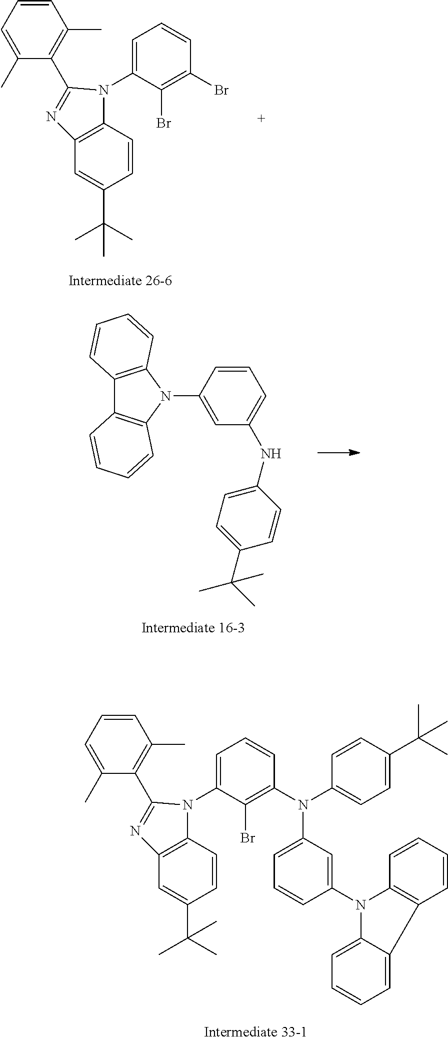 [SMILES: Cc1cccc(C)c1-c1nc2cc(C(C)(C)C)ccc2n1-c1cccc(Br)c1Br ; CC(C)(C)c1ccc(Nc2cccc(-n3c4ccccc4c4ccccc43)c2)cc1 ; Cc1cccc(C)c1-c1nc2cc(C(C)(C)C)ccc2n1-c1cccc(N(c2ccc(C(C)(C)C)cc2)c2cccc(-n3c4ccccc4c4ccccc43)c2)c1Br]

15.03 g (29.3 mmol) of Intermediate 26-2, 12.43 g (32.7 mmol) of Intermediate 16-3 and 7.05 g (73.3 mmol) of sodium tert-butoxide were suspended in 500 ml of toluene. The mixture was evacuated and backfilled with argon 4 times. 0.695 g (2.347 mmol) of tri-tert-butylphosphonium tetrafluoroborate and 0.537 g (0.587 mmol) of Pd₂(dba)₃ were added and the reaction mixture was heated at 85° C. for 5 hours, then cooled to room temperature. 200 ml of water were added and stirred. The phases were separated, the water phase was extracted with ethyl acetate, the combined organic phases were washed with water and brine, dried over magnesium sulfate, filtered and reduced under vacuum. The crude product was purified by column chromatography (heptane/ethyl acetate) to give 22.4 g (93% yield) of Intermediate 33-1.

¹H NMR (300 MHz, Methylene Chloride-d2) δ 8.14 (dt, J=7.7, 1.0 Hz, 2H), 7.84 (dd, J=1.9, 0.6 Hz, 1H), 7.49-7.37 (m, 5H), 7.36-7.21 (m, 7H), 7.15 (ddd, J=7.8, 2.0, 0.9 Hz, 1H), 7.12-6.90 (m, 8H), 6.72 (d, J=7.4 Hz, 1H), 2.18 (s, 3H), 1.93 (s, 3H), 1.42 (s, 9H), 1.31 (s, 9H).

### Compound 33

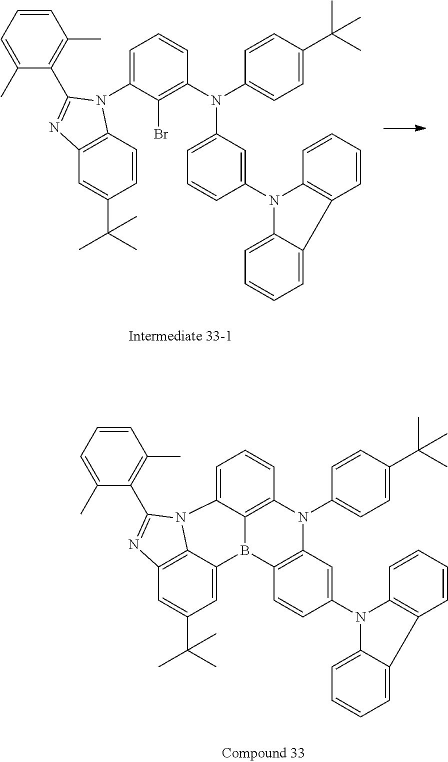 [SMILES: Cc1cccc(C)c1-c1nc2cc(C(C)(C)C)ccc2n1-c1cccc(N(c2ccc(C(C)(C)C)cc2)c2cccc(-n3c4ccccc4c4ccccc43)c2)c1Br ; Cc1cccc(C)c1-c1nc2cc(C(C)(C)C)cc3c2n1-c1cccc2c1B3c1ccc(-n3c4ccccc4c4ccccc43)cc1N2c1ccc(C(C)(C)C)cc1]

14.5 g (17.64 mmol) of Intermediate 33-1 were dissolved in 216 ml of water-free tert-butylbenzene. 18.57 ml (35.3 mmol) of tert-butyllithium (1.9M in pentane) were slowly added at −15° C. and stirred for 15 minutes. The yellowish solution was cooled down to −50° C. and 3.34 ml (35.3 mmol) of tribromoborane were added. The reaction mixture was warmed up to room temperature, stirred for 15 minutes and then cooled down to 0° C. 36.96 ml (211.8 mmol) of N,N-diisopropylethylamine were added in 6 portions within 30 hours while heating at 165° C. After a total reaction time of 32 hours, the yellow suspension was cooled down to room temperature and treated with 50 ml of 10% aqueous sodium acetate solution and stirred overnight. 200 ml of heptane were added to the suspension and stirred for 1 hour, then it was filtered and the residue washed with water, heptane and ethanol. The isolated solid was purified by column chromatography (dichloromethane). The product was dissolved in 100 ml of dichloromethane and 50 ml of heptane were added. The solution was concentrated under vacuum to a volume of 50 ml and the resulting suspension filtered and washed with heptane. The product was again dissolved in 50 ml of dichloromethane and 50 ml of ethyl acetate were added. The solution was concentrated under vacuum to a volume of 50 ml and the resulting suspension filtered, washed with ethyl acetate and dried to give 1.75 g (13.2% yield) of Compound 33.

¹H NMR (300 MHz, Methylene Chloride-d2) δ 9.30 (d, J=8.4 Hz, 1H), 8.94 (d, J=1.7 Hz, 1H), 8.21 (d, J=1.6 Hz, 1H), 8.15-8.08 (m, 2H), 7.71-7.66 (m, 2H), 7.64 (dd, J=8.3, 1.9 Hz, 1H), 7.55-7.50 (m, 2H), 7.48-7.21 (m, 10H), 7.02 (d, J=1.9 Hz, 1H), 6.69 (d, J=8.0 Hz, 1H), 6.60 (d, J=8.5 Hz, 1H), 2.14 (s, 6H), 1.65 (s, 9H), 1.34 (s, 9H).

### Compound 34

### Intermediate 34-1

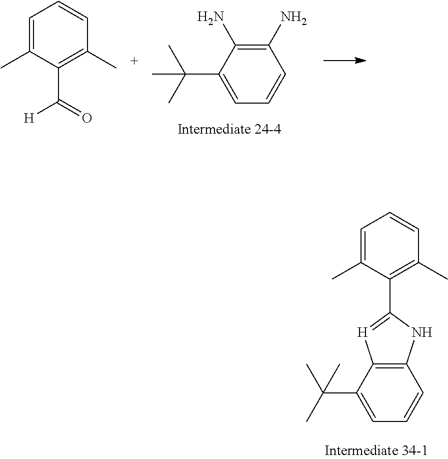

To 12.50 g (72.5 mmol) of Intermediate 24-4 in 150 ml of N,N-dimethylacetamide, 7.55 g (72.5 mmol) of sodium bisulfite were added and heated to 100° C. 9.93 g (72.5 mmol) of 2,6-dimethylbenzaldehyde in 60 ml of N,N-dimethylacetamide were added dropwise at 100° C., then it was stirred at this temperature for 5 hours. The reaction mixture was cooled to room temperature and poured on 500 ml of water and stirred. The suspension was filtered and washed with 300 ml of water and 400 ml of heptane. The crude product was suspended in 100 ml of heptane, filtered, washed with 100 ml of heptane and dried to give 12.75 g (63% yield) of Intermediate 34-1.

¹H NMR (300 MHz, DMSO-d6) δ 12.32 (d, J=45.8 Hz, 1H), 7.37-7.28 (m, 2H), 7.22-7.00 (m, 4H), 2.12 (d, J=13.6 Hz, 6H), 1.49 (d, J=33.3 Hz, 9H).

### Intermediate 34-2

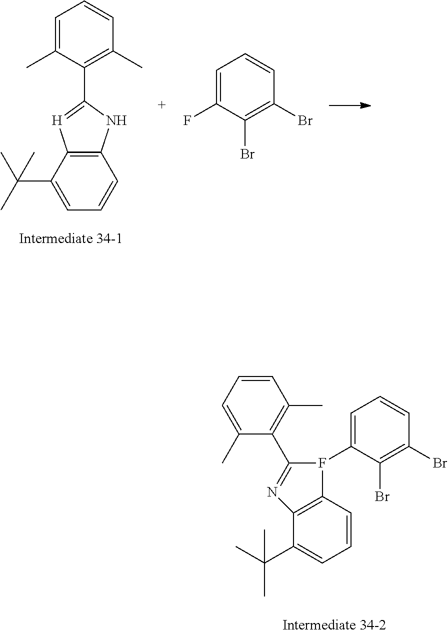

11.9 g (42.7 mmol) of Intermediate 34-1 and 10.85 g (42.7 mmol) of 1,2-dibromo-3-fluorobenzene were dissolved in 250 ml of N,N-dimethylformamide. 45.4 g (214 mmol) of potassium phosphate tribasic were added. The reaction mixture was heated at 150° C. (outside temperature) for 1.5 hours, then cooled to room temperature and filtered. The filtrate was concentrated under vacuum and the isolated solid was dissolved in 100 ml of ethyl acetate, then 60 ml of water were added. The phases were separated, the water phase was extracted with ethylaceteate, the combined organic phases were washed with water and brine, dried over magnesium sulfate, filtered and the solvent was evaporated under reduced pressure. The isolated solid was purified by column chromatography (heptane:ethyl acetate) to give 10.8 g (48% yield) of Intermediate 34-2.

¹H NMR (300 MHz, Methylene Chloride-d2) δ 7.68 (dd, J=8.0, 1.6 Hz, 1H), 7.27-7.07 (m, 5H), 7.05-6.94 (m, 3H), 2.22 (s, 3H), 2.11 (s, 3H), 1.65 (s, 9H).

### Intermediate 34-3

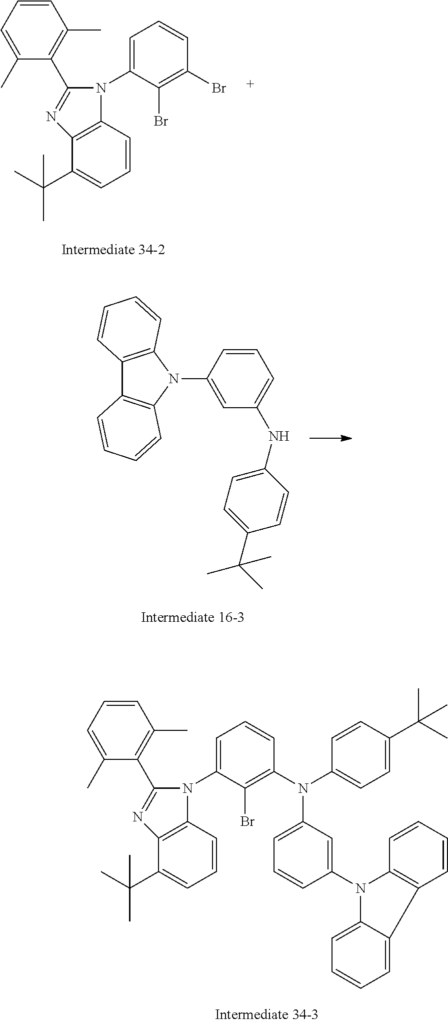 [SMILES: Cc1cccc(C)c1-c1nc2c(C(C)(C)C)cccc2n1-c1cccc(Br)c1Br ; CC(C)(C)c1ccc(Nc2cccc(-n3c4ccccc4c4ccccc43)c2)cc1 ; Cc1cccc(C)c1-c1nc2c(C(C)(C)C)cccc2n1-c1cccc(N(c2ccc(C(C)(C)C)cc2)c2cccc(-n3c4ccccc4c4ccccc43)c2)c1Br]

6.0 g (11.71 mmol) of Intermediate 34-2, 4.88 g (12.30 mmol) of Intermediate 16-3 and 2.81 g (29.3 mmol) of sodium tert-butoxide were suspended in 220 ml of toluene. The mixture was evacuated and backfilled with argon 4 times. 0.277 g (0.937 mmol) of tri-tert-butylphosphonium tetrafluoroborate and 0.215 g (0.234 mmol) of Pd₂(dba)₃ were added and the reaction mixture was heated at 110° C. for 1 hour, then cooled to room temperature. 100 ml of water were added and stirred. The phases were separated, the water phase was extracted with ethyl acetate, the combined organic phases were washed with water and brine, dried over magnesium sulfate, filtered and the solvent was evaporated under reduced pressure. The crude product was purified by column chromatography (heptane/dichloromethane) to give 7.05 g (72% yield) of Intermediate 34-3.

¹H NMR (300 MHz, Methylene Chloride-d2) δ 8.14 (dt, J=7.7, 1.1 Hz, 2H), 7.49-7.23 (m, 11H), 7.23-6.87 (m, 11H), 6.74 (d, J=7.4 Hz, 1H), 2.17 (s, 3H), 1.95 (s, 3H), 1.63 (s, 9H), 1.31 (s, 9H).

### Compound 34

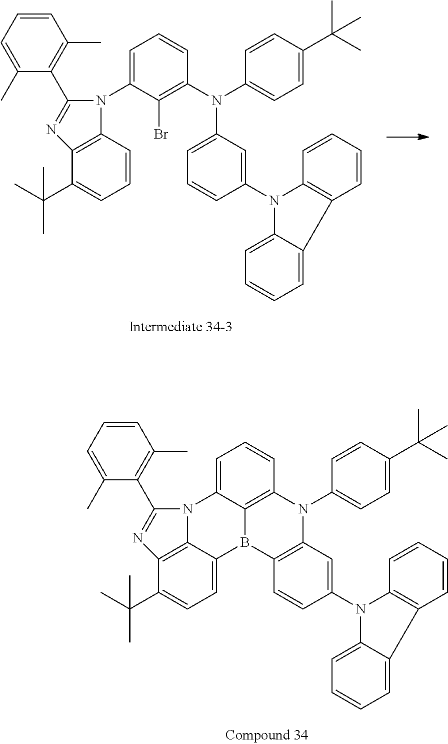 [SMILES: Cc1cccc(C)c1-c1nc2c(C(C)(C)C)cccc2n1-c1cccc(N(c2ccc(C(C)(C)C)cc2)c2cccc(-n3c4ccccc4c4ccccc43)c2)c1Br ; Cc1cccc(C)c1-c1nc2c(C(C)(C)C)ccc3c2n1-c1cccc2c1B3c1ccc(-n3c4ccccc4c4ccccc43)cc1N2c1ccc(C(C)(C)C)cc1]

7.02 g (8.54 mmol) of Intermediate 34-3 were dissolved in 290 ml of water-free tert-butylbenzene. 8.99 ml (17.08 mmol) of tert-butyllithium (1.9M in pentane) were slowly added at 0° C. and stirred for 1 hour. The yellowish solution was cooled down to −35° C. and 1.61 ml (17.08 mmol) of tribromoborane were added. The reaction mixture was warmed up to room temperature, stirred for 1 hour and then cooled down to 0° C. 6.0 ml (34.4 mmol) of N,N-diiso-propylethylamine were added and the reaction mixture was heated at 165° C. for 3 hours. 0.60 ml (34.4 mmol) of N,N-diisopropylethylamine were added again and the reaction mixture was heated at 165° C. for 16 hours. The yellow suspension was cooled down to room temperature and treated with 50 ml of 10% aqueous sodium acetate solution, stirred and extracted with 100 ml of ethyl acetate. The organic layer was separated, washed with water, dried over magnesium sulfate, filtered and concentrated under vacuum. The isolated product was purified by column chromatography (heptane/dichloromethane) to give 0.631 g (10% yield) of Compound 34.

LC-MS: 749.4 [M−H]⁻

### Compound 35

### Intermediate 35-1

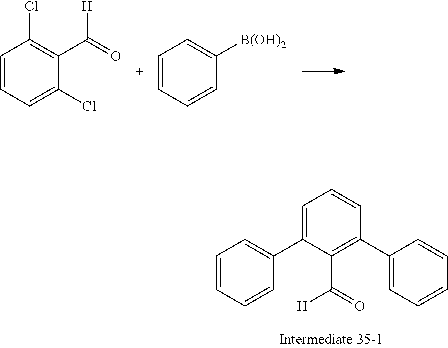

20.0 g (112 mmol) of 2,6-dichlorobenzaldehyde, 57.36 g (470 mmol) of phenylboronic acid and 124 g (381 mmol) of cesium carbonate were suspended in 285 ml of dioxane. The mixture was evacuated and backfilled with argon 4 times. 5.03 g (17.92 mmol) of tricyclohexylphosphine in 25 ml of toluene and 6.56 g (7.17 mmol) of Pd₂(dba)₃ were added and the reaction mixture was heated at 100° C. for 18 hours. The cooled reaction mixture was diluted with 100 ml of water and 100 ml of ethyl acetate, the phases were separated and the water phase extracted with ethyl acetate. The combined organic phases were washed with water and brine, dried over magnesium sulfate, filtered and concentrated under vacuum. The isolated product was purified by column chromatography (heptane/ethyl acetate) to give 25.92 g (88% yield) of Intermediate 35-1.

¹H NMR (300 MHz, Methylene Chloride-d2) δ 9.93 (s, 1H), 7.61 (dd, J=8.1, 7.2 Hz, 1H), 7.48-7.38 (m, 8H), 7.37-7.32 (m, 4H).

### Intermediate 35-2

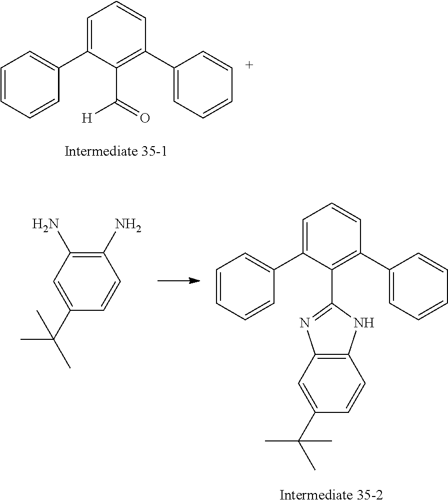 [SMILES: O=Cc1c(-c2ccccc2)cccc1-c1ccccc1 ; CC(C)(C)c1ccc(N)c(N)c1 ; CC(C)(C)c1ccc2[nH]c(-c3c(-c4ccccc4)cccc3-c3ccccc3)nc2c1]

To 18.75 g (114 mmol) of 4-(tert-butyl)benzene-1,2-diamine in 455 ml of N,N-dimethylacetamide, 13.77 g (132 mmol) of sodium bisulfite were added and heated to 100° C. 25.91 g (98 mmol) of Intermediate 35-1 in 270 ml of N,N-dimethylacetamide were added dropwise at 100° C., then it was stirred at this temperature for 18 hours. The reaction mixture was cooled to room temperature and poured on 1.75 l of water and stirred. The suspension was filtered and washed with 750 ml of water and 500 ml of heptane and dried. The isolated solid was suspended in 600 ml of dichloromethane and sonicated in the ultrasonic bath for 1 hour. The precipitate was filtered and washed with dichloromethane and dried to give 36.5 g (93% yield) of Intermediate 35-2.

¹H NMR (300 MHz, DMSO-d6) δ 12.01 (d, J=13.6 Hz, 1H), 7.68 (ddd, J=8.2, 7.3, 1.0 Hz, 1H), 7.47 (dd, J=7.5, 1.1 Hz, 2H), 7.40-7.32 (m, 1H), 7.24-7.08 (m, 12H), 1.27 (d, J=4.0 Hz, 9H).

### Intermediate 35-3

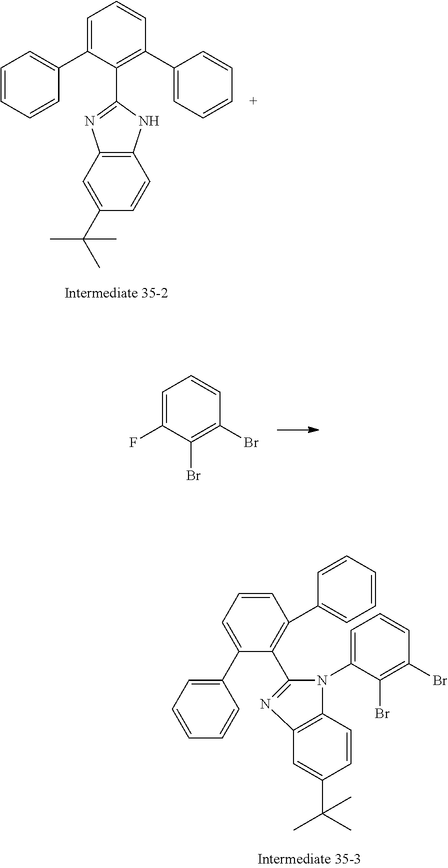 [SMILES: CC(C)(C)c1ccc2[nH]c(-c3c(-c4ccccc4)cccc3-c3ccccc3)nc2c1 ; Fc1cccc(Br)c1Br ; CC(C)(C)c1ccc2c(c1)nc(-c1c(-c3ccccc3)cccc1-c1ccccc1)n2-c1cccc(Br)c1Br]

15.25 g (37.6 mmol) of Intermediate 35-2 and 14.31 g (56.4 mmol) of 1,2-dibromo-3-fluorobenzene were suspended in 450 ml of N,N-dimethylformamide. 39.9 g (188 mmol) of potassium phosphate tribasic were added. The reaction mixture was heated at 150° C. (outside temperature) for 6 hours, then cooled to room temperature and filtered. The filtrate was concentrated under vacuum and the isolated solid was suspended in 200 ml of dichloromethane, filtered and dried. The isomers were separated by column chromatography (dichloromethane) to give 12.5 g (52% yield) of Intermediate 35-3.

¹H NMR (300 MHz, Methylene Chloride-d₂) δ 7.81 (dd, J=1.9, 0.6 Hz, 1H), 7.59-7.54 (m, 1H), 7.52 (d, J=7.7 Hz, 1H), 7.49-7.43 (m, 2H), 7.40 (dd, J=7.8, 1.4 Hz, 1H), 7.37-7.31 (m, 2H), 7.28-7.19 (m, 8H), 6.93 (t, J=8.0 Hz, 1H), 6.68 (dd, J=8.5, 0.7 Hz, 1H), 6.33 (dd, J=7.9, 1.5 Hz, 1H), 1.40 (s, 9H).

### Intermediate 35-4

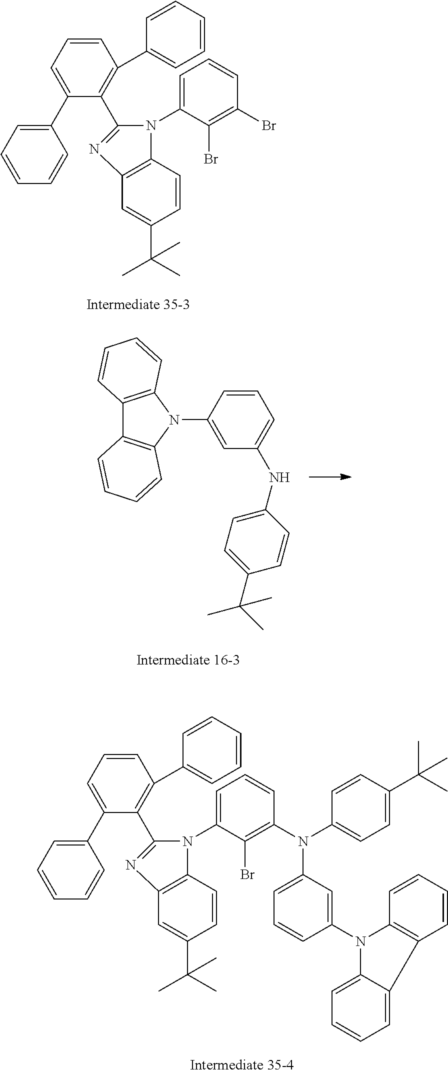 [SMILES: CC(C)(C)c1ccc2c(c1)nc(-c1c(-c3ccccc3)cccc1-c1ccccc1)n2-c1cccc(Br)c1Br ; CC(C)(C)c1ccc(Nc2cccc(-n3c4ccccc4c4ccccc43)c2)cc1 ; CC(C)(C)c1ccc(N(c2cccc(-n3c4ccccc4c4ccccc43)c2)c2cccc(-n3c(-c4c(-c5ccccc5)cccc4-c4ccccc4)nc4cc(C(C)(C)C)ccc43)c2Br)cc1]

0.359 g (0.564 mmol) of Intermediate 35-3, 0.236 g (0.592 mmol) of Intermediate 16-3 and 0.136 g (1.41 mmol) of sodium tert-butoxide were suspended in 10 ml of toluene. The mixture was evacuated and backfilled with argon 4 times. 0.013 g (0.045 mmol) of tri-tert-butylphosphonium tetrafluoroborate and 0.010 g (0.011 mmol) of Pd₂(dba)₃ were added and the reaction mixture was heated at 80° C. for 5 hours, then cooled to room temperature. 10 ml of water were added and stirred. The phases were separated, the water phase was extracted with ethyl acetate, the combined organic phases were washed with water and brine, dried over magnesium sulfate, filtered and the solvent was evaporated under vacuum. The crude product was purified by column chromatography (heptane/ethyl acetate) to give 0.347 g (65% yield) of Intermediate 35-4.

¹H NMR (300 MHz, Methylene Chloride-od) δ 8.13 (dt, J=7.6, 1.1 Hz, 2H), 7.80 (d, J=1.7 Hz, 1H), 7.49-7.20 (m, 19H), 7.18-6.92 (m, 10H), 6.86 (d, J=7.6 Hz, 1H), 6.52 (d, J=8.6 Hz, 1H), 6.22 (dd, J=7.9, 1.6 Hz, 1H), 1.39 (s, 9H), 1.33 (s, 9H).

### Compound 35

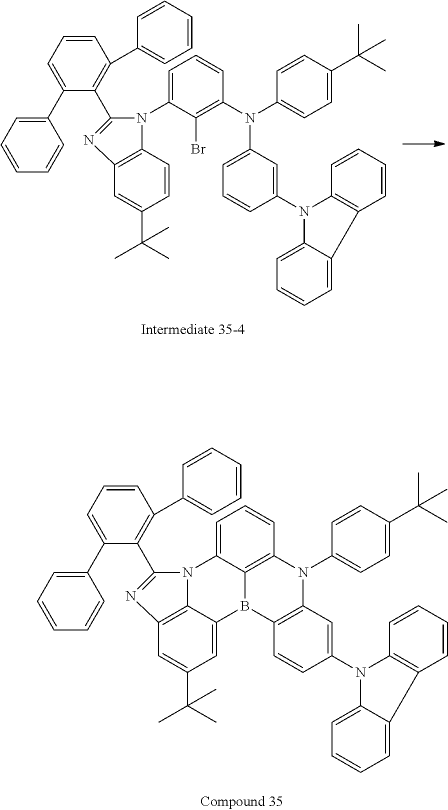 [SMILES: CC(C)(C)c1ccc(N(c2cccc(-n3c4ccccc4c4ccccc43)c2)c2cccc(-n3c(-c4c(-c5ccccc5)cccc4-c4ccccc4)nc4cc(C(C)(C)C)ccc43)c2Br)cc1 ; CC(C)(C)c1ccc(N2c3cc(-n4c5ccccc5c5ccccc54)ccc3B3c4c2cccc4-n2c(-c4c(-c5ccccc5)cccc4-c4ccccc4)nc4cc(C(C)(C)C)cc3c42)cc1]

8.22 g (8.69 mmol) of Intermediate 35-4 were dissolved in 175 ml of water-free tert-butylbenzene. 10.22 ml (17.38 mmol) of tert-butyllithium (1.7M in pentane) were slowly added at 0° C. and stirred for 20 minutes. The yellowish solution was cooled down to −50° C. and 1.64 ml (17.38 mmol) of tribromoborane were added. The reaction mixture was warmed up to room temperature, stirred for 30 minutes and then cooled down to 0° C. 7.59 ml (43.4 mmol) of N,N-diisopropylethylamine were added in 3 portions within 18 hours while heating at 165° C. After a total reaction time of 24 hours, the yellow suspension was cooled down to room temperature and treated with 20 ml of 10% aqueous sodium acetate solution and stirred overnight. The phases were separated and the water phase was extracted with ethyl acetate. The combined organic phases were washed with water and brine, dried over magnesium sulfate, filtered and concentrated under vacuum. The isolated product was precipitated in heptane and then purified by column chromatography (dichloromethane) to give 3.45 g (44%) of Compound 35.

¹H NMR (300 MHz, Methylene Chloride-d2) δ 9.18 (d, J=8.3 Hz, 1H), 8.78 (d, J=1.7 Hz, 1H), 8.10 (dt, J=7.6, 1.1 Hz, 2H), 8.00 (d, J=1.6 Hz, 1H), 7.79 (dd, J=8.3, 7.1 Hz, 1H), 7.72-7.66 (m, 2H), 7.64-7.47 (m, 5H), 7.42-7.23 (m, 7H), 7.22-7.15 (m, 4H), 7.06-6.92 (m, 8H), 6.56 (dd, J=8.6, 0.7 Hz, 1H), 1.57 (s, 9H), 1.34 (s, 9H).

### Compound 36

### Intermediate 36-1

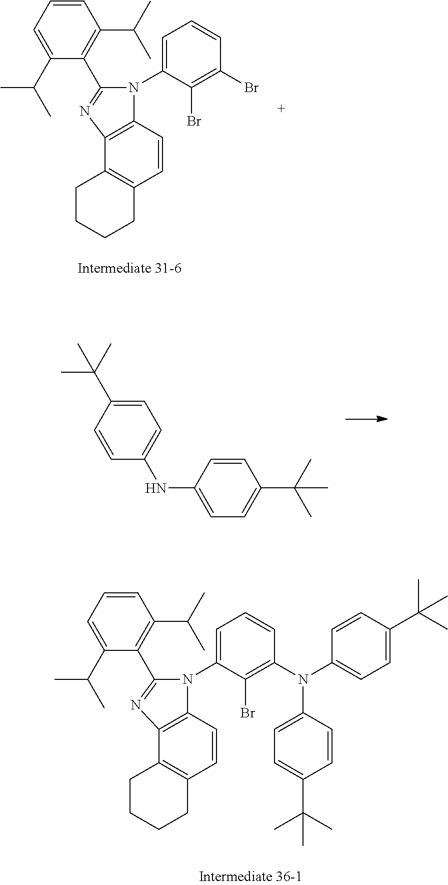 [SMILES: CC(C)c1cccc(C(C)C)c1-c1nc2c3c(ccc2n1-c1cccc(Br)c1Br)CCCC3 ; CC(C)(C)c1ccc(Nc2ccc(C(C)(C)C)cc2)cc1 ; CC(C)c1cccc(C(C)C)c1-c1nc2c3c(ccc2n1-c1cccc(N(c2ccc(C(C)(C)C)cc2)c2ccc(C(C)(C)C)cc2)c1Br)CCCC3]

8.0 g (14.1 mmol) of Intermediate 31-6, 4.17 g (14.8 mmol) of bis(4-(tert-butyl)phenyl)amine, 259 mg (0.28 mmol) of tris(dibenzylideneacetone)dipalladium(0), 328 mg (1.13 mmol) of tri-tert-butylphosphonium tetrafluoroborate, and 3.39 g (35.3 mmol) of sodium tert-butoxide were suspended in 100 ml of o-xylene. The dark suspension was 3 times evacuated and backfilled with argon and heated at 124° C. during 2 hours. The reaction mixture was cooled down and filtered through a 3 cm layer of silica gel followed by rinsing the silica gel layer with 200 ml of toluene. The collected eluents were concentrated under vacuum and the product purified by MPLC with the CombiFlash Companion (silica gel, dichloromethane). The isolated product was diluted with 20 ml of dichloromethane and 100 ml of methanol and concentrated under vacuum until a precipitate formed. The suspension was further stirred at room temperature during 2 hours. The fine suspension was filtered and the solid washed with 50 ml of methanol to give 6.10 g (56% yield) of Intermediate 36-1 as a white solid.

¹H NMR (300 MHz, CD₂Cl₂) δ 7.44 (t, 1H), 7.34-7.23 (m, 7H), 7.15 (dd, 1H), 7.07-6.94 (m, 2H), 6.92-6.79 (m, 5H), 3.22 (broad signal, 2H), 2.94 (broad signal, 2H), 2.86-2.69 (m, 1H), 2.64-2.45 (m, 1H), 2.07-1.86 (m, 4H), 1.38-1.25 (2 d, 6H), 1.35 (s, 18H), 1.07 (d, 3H), 0.84 (d, 3H).

### Compound 36

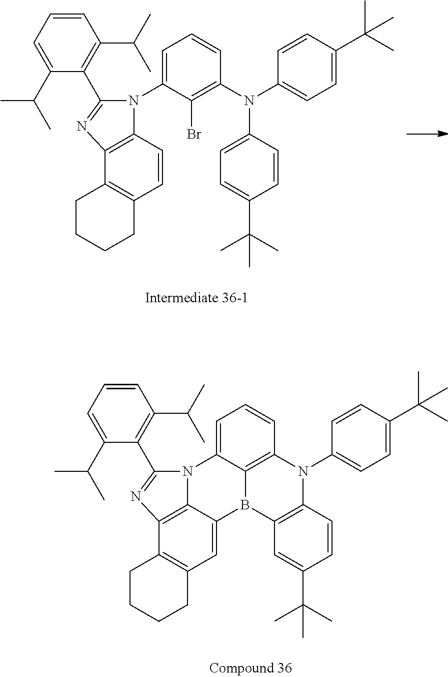

3.00 g (3.91 mmol) of Intermediate 36-1 were dissolved in 40 ml of water-free tert-butylbenzene. 4.12 ml of tert-butyllithium (1.9 M in pentane) were slowly added at −3° C. and stirred up to a temperature of 16° C. during 25 minutes. The brown solution was cooled down to −49° C. and 0.74 ml (7.82 mmol) of tribromoborane were added. The brown suspension was warmed up to 19° C. during 1 hour and cooled down to −2° C. 1.37 ml (7.82 mmol) of N,N-diisopropylethylamine were slowly added and the reaction mixture heated at 157° C. during 22 hours. The orange suspension was cooled down and treated with 50 ml of water and 50 ml of ethyl acetate. The organic phase was separated and extracted with water (3×40 ml), dried over sodium sulfate and concentrated under vacuum. The solid was suspended in 40 ml ethyl acetate and stirred at room temperature during 30 minutes, then filtered and the solid washed with 20 ml of ethyl acetate and 30 ml of heptane. The solid was suspended in 40 ml of dichloromethane and 40 ml of ethyl acetate and concentrated under vacuum with heating to a volume of 30 ml. The suspension was filtered and the solid washed with 30 ml of ethyl acetate. The solid was purified by MPLC with the CombiFlash Companion (silica gel, dichloromethane) to give 0.43 g (16% yield) of Compound 36 as a yellow solid.

¹H NMR (400 MHz, CD₂Cl₂) δ 9.11 (d, 1H), 8.56 (s, 1H), 7.78-7.72 (m, 2H), 7.67-7.59 (m, 2H), 7.42 (d, 2H), 7.32-7.26 (m, 2H), 7.19 (t, 1H), 6.79 (d, 1H), 6.71 (dd, 1H), 6.51 (d, 1H), 3.43 (broad signal, 2H), 3.24 (broad signal, 2H), 2.60 (hept, 2H), 2.14-2.01 (m, 4H), 1.53 (s, 9H), 1.49 (s, 9H), 1.20 (d, 6H), 1.01 (d, 6H).

### Compound 37

### Intermediate 37-1

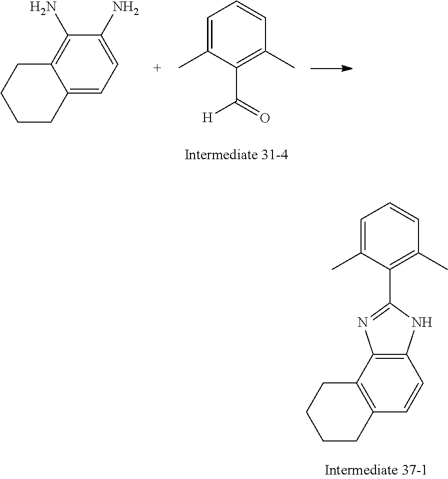

60.0 g (0.37 mol) of Intermediate 31-4 and 38.5 g (0.37 mol) of sodium bisulfite in 250 ml of N,N-dimethylacetamide were heated to 100° C. 49.6 g (0.37 mol) of 2,6-dimethylbenzaldehyde in 50 ml of N,N-dimethylacetamide were dropwise added during 15 minutes, and heating continued at 102° C. during 90 minutes. The reaction mixture was poured into 1000 ml of water, and the resulting suspension filtered and the solid washed with 500 ml of water. The solid was suspended in 300 ml of dichloromethane and 600 ml of heptane and the suspension stirred during 30 minutes. The suspension was filtered and the solid washed with 200 ml of heptane to give 94.6 g (93% yield) of Intermediate 37-1 as a solid.

¹H NMR (300 MHz, DMSO-d₆) δ 12.31 (br. signal, 1H), 7.43-7.26 (m, 2H), 7.26-7.13 (m, 2H), 6.95-6.85 (m, 1H), 2.93-2.76 (m, 4H), 2.10 (s, 6H), 1.91-1.74 (m, 4H).

### Intermediate 37-2

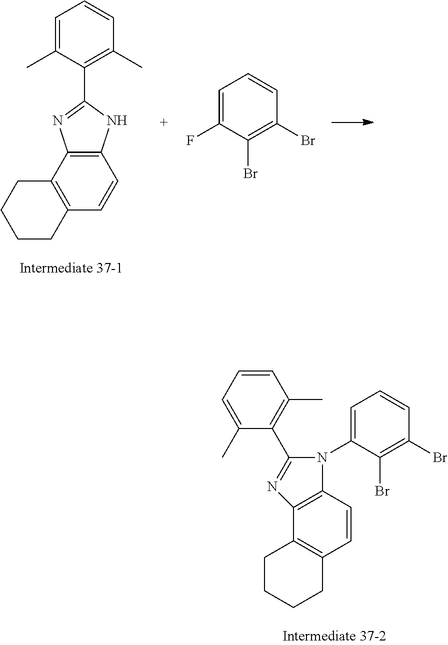 [SMILES: Cc1cccc(C)c1-c1nc2c3c(ccc2[nH]1)CCCC3 ; Fc1cccc(Br)c1Br ; Cc1cccc(C)c1-c1nc2c3c(ccc2n1-c1cccc(Br)c1Br)CCCC3]

21.6 g (78 mmol) of Intermediate 37-1, 21.8 g (86 mmol) of 2,3-dibromofluorobenzene, and 66.4 g (313 mmol) of potassium phosphate were suspended in 200 ml of N,N-dimethyl-formamide, followed by heating at 143° C. during 4 hours. The reaction mixture was cooled down to room temperature and poured into 200 ml of water. The resulting suspension was filtered and the solid washed with water. The solid was suspended in 200 ml of acetone and cooled to 0° C. The suspension was filtered, and the solid washed with ice-cold acetone to give 25.6 g (64% yield) of Intermediate 37-2.

¹H NMR (400 MHz, CD₂Cl₂) δ 7.70 (dd, 1H), 7.22 (t, 1H), 7.19-7.09 (m, 2H), 7.10-7.03 (m, 2H), 7.00 (d, 1H), 6.90 (d, 1H), 3.30-3.20 (m, 2H), 3.02-2.88 (m, 2H), 2.26 (s, 3H), 2.13 (s, 3H), 2.04-1.88 (m, 4H).

### Intermediate 37-3

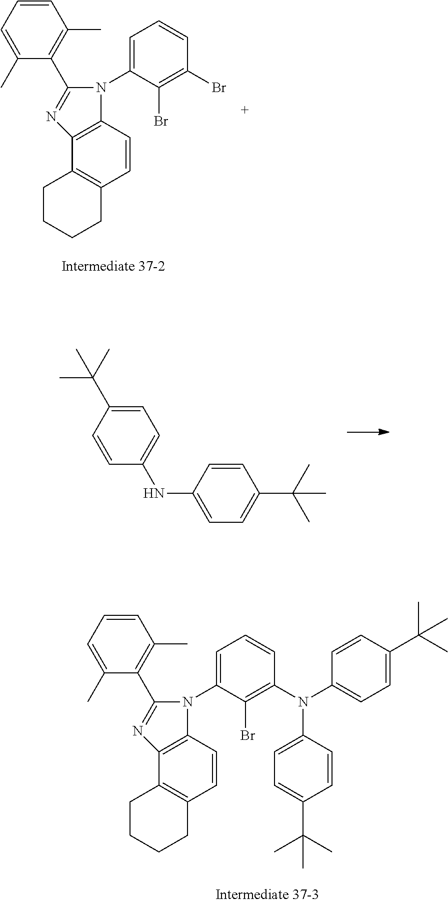 [SMILES: Cc1cccc(C)c1-c1nc2c3c(ccc2n1-c1cccc(Br)c1Br)CCCC3 ; CC(C)(C)c1ccc(Nc2ccc(C(C)(C)C)cc2)cc1 ; Cc1cccc(C)c1-c1nc2c3c(ccc2n1-c1cccc(N(c2ccc(C(C)(C)C)cc2)c2ccc(C(C)(C)C)cc2)c1Br)CCCC3]

12.0 g (23.5 mmol) of Intermediate 37-2, 6.95 g (24.7 mmol) of bis(4-(tert-butyl)phenyl)amine, 431 mg (0.47 mmol) of tris(dibenzylideneacetone)dipalladium(0), 546 mg (1.88 mmol) of tri-tert-butylphosphonium tetrafluoroborate, and 5.65 g (58.8 mmol) of sodium tert-butoxide were suspended in 150 ml of toluene. The dark suspension was three times evacuated and backfilled with argon and heated at 107° C. during 1 hour. The reaction mixture was cooled down and filtered through a 3 cm layer of silica gel followed by rinsing the silica gel layer with 100 ml of toluene. The collected eluents were treated with 50 ml of 5% aqueous sodium cyanide solution and the mixture vigorously stirred at 50° C. during 1 hour. The organic phase was separated and extracted with water (2×50 ml), then dried over sodium sulfate and concentrated under vacuum. The product was further purified by MPLC with the CombiFlash Companion (silica gel, dichloromethane). The isolated product was recrystallized from 60 ml of methanol to give 11.6 g (69% yield) of Intermediate 37-3 as a white solid.

¹H NMR (400 MHz, CD₂Cl₂) δ 7.34-7.22 (m, 7H), 7.14 (dd, 1H), 7.08-6.99 (m, 3H), 6.92 (d, 1H), 6.89-6.82 (m, 4H), 3.31-3.19 (m, 2H), 3.01-2.89 (m, 2H), 2.24 (s, 3H), 2.10 (s, 3H), 2.04-1.86 (m, 4H), 1.36 (s, 18H).

### Compound 37

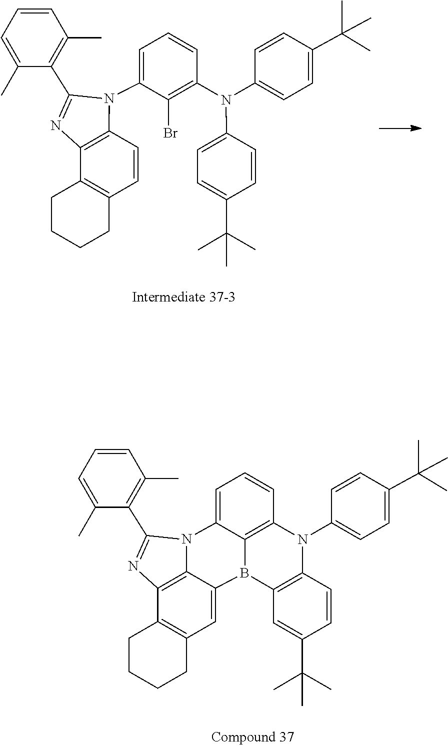 [SMILES: Cc1cccc(C)c1-c1nc2c3c(ccc2n1-c1cccc(N(c2ccc(C(C)(C)C)cc2)c2ccc(C(C)(C)C)cc2)c1Br)CCCC3 ; Cc1cccc(C)c1-c1nc2c3c(cc4c2n1-c1cccc2c1B4c1cc(C(C)(C)C)ccc1N2c1ccc(C(C)(C)C)cc1)CCCC3]

5.00 g (7.03 mmol) of Intermediate 37-3 were dissolved in 80 ml of water-free tert-butylbenzene. 7.4 ml of tert-butyllithium (1.9 M in pentane) were slowly added at −3° C. and stirred up to a temperature of 3° C. during 15 minutes. The brown solution was cooled down to −49° C. and 1.33 ml (14.1 mmol) of tribromoborane were added. The brown suspension was warmed up to 4° C. during 10 minutes and cooled down to −2° C. 2.45 ml (14.1 mmol) of N,N-diisopropylethylamine were slowly added and the yellow suspension heated at 146° C. during 21 hours. The orange suspension was cooled down and treated with 50 ml of water and 50 ml of ethyl acetate. The organic phase was separated and extracted with water (3×40 ml), dried over sodium sulfate and concentrated under vacuum. The yellow solid was further purified by MPLC with the CombiFlash Companion (silica gel, dichloromethane). The product was dissolved in 30 ml of dichloromethane and mixed with 50 ml of MeOH, followed by stirring for 15 minutes. The suspension was filtered and the solid washed with 30 ml of methanol to give 0.91 g (20% yield) of Compound 37 as a yellow solid.

ESI-MS (positive, m/z): exact mass of C₄₅H₄₆BN₃=639.38; found 640.6 [M+1]⁺.

### Compound 38

### Intermediate 38-1

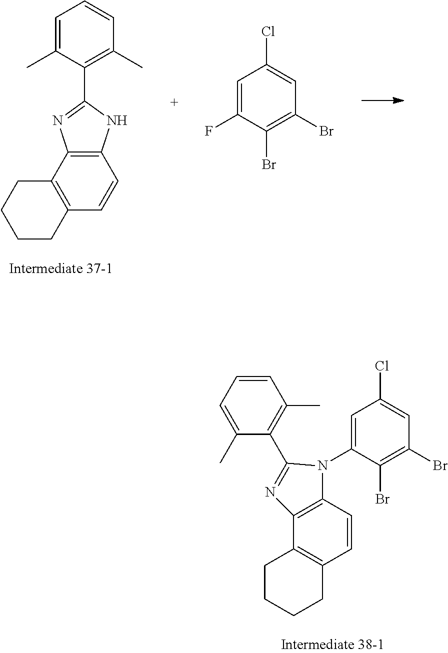 [SMILES: Cc1cccc(C)c1-c1nc2c3c(ccc2[nH]1)CCCC3 ; Fc1cc(Cl)cc(Br)c1Br ; Cc1cccc(C)c1-c1nc2c3c(ccc2n1-c1cc(Cl)cc(Br)c1Br)CCCC3]

10.8 g (38.9 mmol) of Intermediate 37-1, 22.4 g (78.0 mmol) of 1,2-dibromo-5-chloro-3-fluorobenzene and 41.3 g (0.19 mol) of potassium phosphate were suspended in 300 ml of N,N-dimethylformamide, followed by heating at 74° C. during 19 hours. The reaction mixture was cooled down to room temperature and poured into 900 ml of water, and extracted two times with 300 ml of dichloromethane. The organic phase was washed first with water (2×300 ml), then with 300 ml of saturated aqueous sodium chloride solution, followed by drying over sodium sulfate, and concentration under vacuum. The product was further purified by MPLC with the CombiFlash Companion (silica gel, heptane/0-20% gradient of ethyl acetate). The isolated product dissolved in 100 ml of dichloromethane and 200 ml of heptane. The solution was concentrated under vacuum until a suspension formed. The suspension was stirred at room temperature overnight and filtered to give 16.1 g (76% yield) of Intermediate 38-1 as a white solid.

¹H NMR (300 MHz, CDCl₃) δ 7.67 (d, 1H), 7.21 (t, 1H), 7.10 (t, 2H), 7.04-6.95 (m, 2H), 6.90 (d, 1H), 3.38-3.22 (m, 2H), 3.03-2.86 (m, 2H), 2.27 (s, 3H), 2.12 (s, 3H), 2.05-1.86 (m, 4H).

### Intermediate 38-2

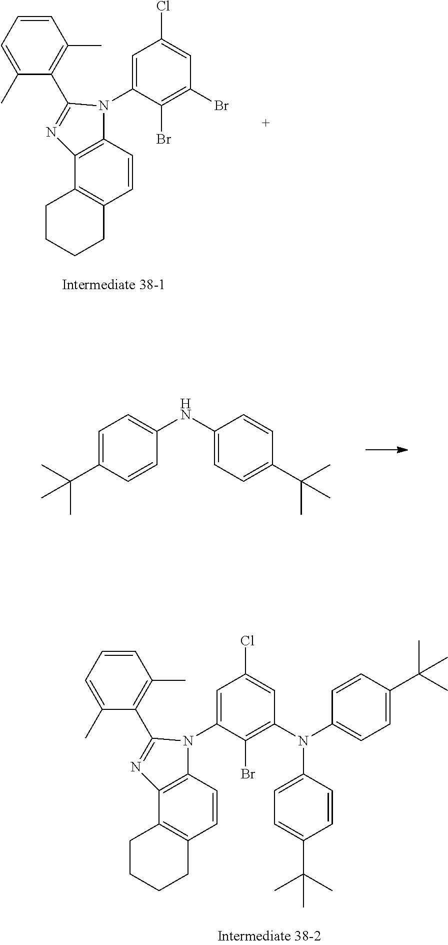 [SMILES: Cc1cccc(C)c1-c1nc2c3c(ccc2n1-c1cc(Cl)cc(Br)c1Br)CCCC3 ; CC(C)(C)c1ccc(Nc2ccc(C(C)(C)C)cc2)cc1 ; Cc1cccc(C)c1-c1nc2c3c(ccc2n1-c1cc(Cl)cc(N(c2ccc(C(C)(C)C)cc2)c2ccc(C(C)(C)C)cc2)c1Br)CCCC3]

12.0 g (22.0 mmol) of Intermediate 38-1, 6.51 g (23.1 mmol) of bis(4-(tert-butyl)phenyl)amine, 0.41 g (0.44 mmol) of tris(dibenzylideneacetone)dipalladium(0), 0.52 g (1.8 mmol) of tri-tert-butylphosphonium tetrafluoroborate, and 5.29 g (55.1 mmol) of sodium tert-butoxide were suspended in 400 ml of toluene. The dark suspension was three times evacuated and backfilled with argon and heated at 73° C. during 3 hours. 0.2 g of tris(dibenzylideneacetone)dipalladium(0), 0.25 g of tri-tert-butylphosphonium tetrafluoroborate were added and heating continued at 73° C. during 1 hour. 100 ml of water and 0.3 g of sodium cyanide were added and stirring continued at 73° C. during 15 minutes. The reaction mixture was cooled down to room temperature and the organic phase extracted with water (2×100 ml) and 100 ml of saturated aqueous sodium chloride solution. The solution was concentrated under vacuum and the product further purified by MPLC with the CombiFlash Companion (silica gel, heptane/10% ethyl acetate). The isolated product was dissolved in 100 ml of hot 2-propanol and concentrated under vacuum until a suspension formed. The suspension was left stirring overnight. The suspension was filtered and the solid washed with a small amount of cold 2-propanol to give 10.8 g (66% yield) of Intermediate 38-2 as a white solid.

¹H NMR (300 MHz, CD₂Cl₂) δ 7.36-7.26 (m, 5H), 7.21 (d, 1H), 7.17 (d, 1H), 7.12-7.03 (m, 2H), 7.00 (d, 1H), 6.92 (d, 1H), 6.87-6.78 (m, 4H), 3.31-3.18 (m, 2H), 3.03-2.85 (m, 2H), 2.23 (s, 3H), 2.10 (s, 3H), 2.02-1.84 (m, 4H), 1.36 (s, 18H).

### Intermediate 38-3

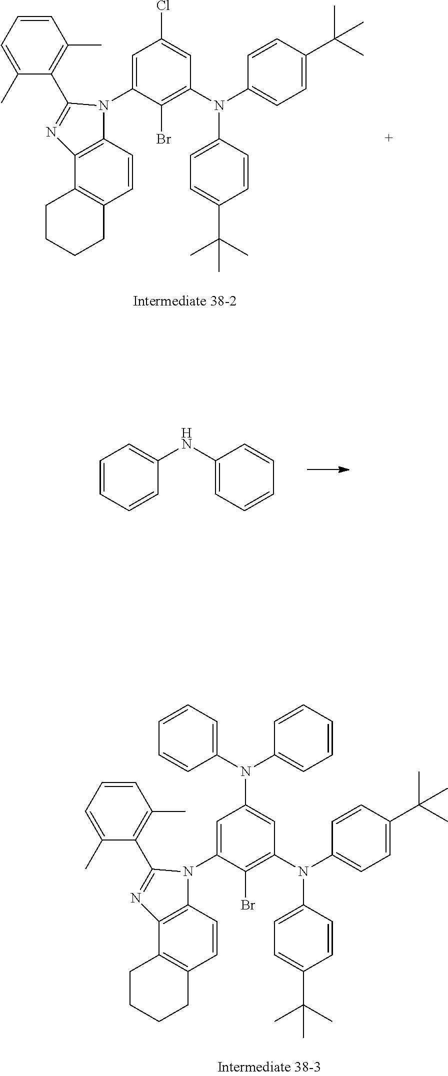 [SMILES: Cc1cccc(C)c1-c1nc2c3c(ccc2n1-c1cc(Cl)cc(N(c2ccc(C(C)(C)C)cc2)c2ccc(C(C)(C)C)cc2)c1Br)CCCC3 ; c1ccc(Nc2ccccc2)cc1 ; Cc1cccc(C)c1-c1nc2c3c(ccc2n1-c1cc(N(c2ccccc2)c2ccccc2)cc(N(c2ccc(C(C)(C)C)cc2)c2ccc(C(C)(C)C)cc2)c1Br)CCCC3]

24.6 g (33.0 mmol) of Intermediate 38-2, 6.30 g (37.2 mmol) of diphenylamine, 0.75 g (0.83 mmol) of tris(dibenzylideneacetone)dipalladium(0), 0.96 g (1.65 mmol) of tri-tert-butyl-phosphonium tetrafluoroborate, and 7.90 g (82.2 mmol) of sodium tert-butoxide were suspended in 250 ml of toluene. The suspension was three times evacuated and backfilled with argon and heated up to 99° C. during 7 hours. 100 ml of water and 0.2 g of sodium cyanide were added and stirring continued at 73° C. during 10 minutes. The reaction mixture was cooled down to room temperature and the organic phase extracted with 200 ml of water and 100 ml of saturated aqueous sodium chloride solution. The organic phase was filtered over a 7 cm layer of silica gel and the silica gel layer rinsed with toluene. The combined eluents were concentrated under vacuum. The product was mixed with 300 ml of ethanol and heated until a suspension formed. The suspension was stirred for 30 minutes at room temperature and the solid separated and washed with a small amount of ethanol. The grey powder was dissolved in 200 ml of dichloromethane and stirred at room temperature during 15 minutes with 200 ml of water, 50 ml of ethanol and 0.6 g of L-cysteine. The organic phase was separated, dried over magnesium sulfate and concentrated under vacuum. The isolated product was dissolved in dichloromethane and ethanol was added. The solution was concentrated under vacuum until a suspension was formed. The suspension was filtered to give 17.6 g (61% yield) of Intermediate 38-3 as a white solid.

¹H NMR (300 MHz, CD₂Cl₂) δ 7.40-7.18 (m, 10H), 7.18-6.98 (m, 6H), 6.94-6.82 (m, 9H), 3.29-3.12 (m, 2H), 3.04-2.84 (m, 2H), 2.09 (s, 3H), 2.03-1.85 (m, 4H), 1.78 (s, 3H), 1.37 (s, 18H).

### Compound 38

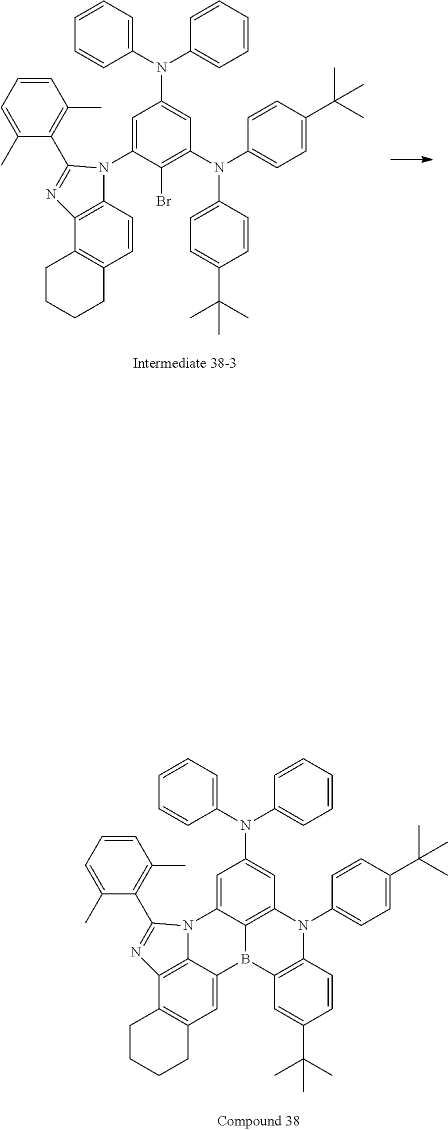 [SMILES: Cc1cccc(C)c1-c1nc2c3c(ccc2n1-c1cc(N(c2ccccc2)c2ccccc2)cc(N(c2ccc(C(C)(C)C)cc2)c2ccc(C(C)(C)C)cc2)c1Br)CCCC3 ; Cc1cccc(C)c1-c1nc2c3c(cc4c2n1-c1cc(N(c2ccccc2)c2ccccc2)cc2c1B4c1cc(C(C)(C)C)ccc1N2c1ccc(C(C)(C)C)cc1)CCCC3]

9.34 g (10.6 mmol) of Intermediate 38-3 were dissolved in 150 ml of water-free tert-butylbenzene. 11.2 ml of tert-butyllithium (1.9 M in pentane) were slowly added at −20° C. and stirred up to room temperature during 1 hour. The orange solution was cooled down to −30° C. and 2.0 ml (21.1 mmol) of tribromoborane were added. The orange suspension was warmed up to room temperature during 30 minutes and cooled down to −10° C. 3.7 ml (21.1 mmol) of N,N-diisopropylethylamine were slowly added and the yellow suspension heated at 152° C. during 21 hours. The orange-yellow suspension was cooled down and treated with 100 ml of 10% aqueous sodium acetate solution. The suspension was heated at 90° C. during 50 minutes and cooled down to room temperature. The suspension was filtered and the solid washed with 50 ml of water, 5 ml of tert-butylbenzene, and twice with 10 ml of ethanol. The yellow solid was further purified by MPLC with the CombiFlash Companion (silica gel, dichloromethane). The isolated product was dissolved in a minimum amount of dichloromethane and ethanol was added. The solution was concentrated under vacuum until a suspension formed. The suspension was filtered and the solid washed twice with 5 ml of ethanol to give 1.60 g (19% yield) of Compound 38 as a yellow solid.

¹H NMR (300 MHz, CD₂Cl₂) δ 9.03 (d, 1H), 8.51 (s, 1H), 7.63-7.44 (m, 3H), 7.26-6.95 (m, 9H), 6.93-6.72 (m, 7H), 6.40 (s, 1H), 5.95 (s, 1H), 3.51-3.29 (m, 2H), 3.29-3.13 (m, 2H), 2.19-1.92 (m, 7H), 1.52 (s, 9H), 1.47 (s, 3H), 1.36 (s, 9H).

### Compound 39

### Intermediate 39-1

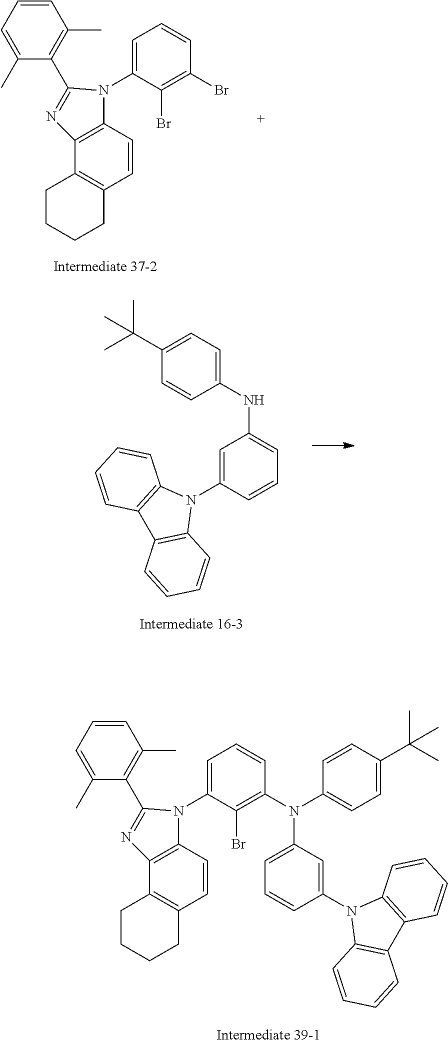 [SMILES: Cc1cccc(C)c1-c1nc2c3c(ccc2n1-c1cccc(Br)c1Br)CCCC3 ; CC(C)(C)c1ccc(Nc2cccc(-n3c4ccccc4c4ccccc43)c2)cc1 ; Cc1cccc(C)c1-c1nc2c3c(ccc2n1-c1cccc(N(c2ccc(C(C)(C)C)cc2)c2cccc(-n4c5ccccc5c5ccccc54)c2)c1Br)CCCC3]

12.5 g (24.5 mmol) of Intermediate 37-2, 9.57 g (24.5 mmol) of Intermediate 16-3, 449 mg (0.49 mmol) of tris(dibenzylideneacetone)dipalladium(0), 569 mg (1.96 mmol) of tri-tert-butylphosphonium tetrafluoroborate, and 5.89 g (61.2 mmol) of sodium tert-butoxide were suspended in 200 ml of o-xylene. The dark suspension was three times evacuated and backfilled with argon and heated at 122° C. during 45 minutes. The reaction mixture was cooled down and filtered through a 3 cm layer of silica gel followed by rinsing the silica gel layer with 100 ml of toluene. The collected eluents were concentrated under vacuum and the product purified by MPLC with the CombiFlash Companion (silica gel, dichloromethane). The isolated product was further purified by MPLC with the CombiFlash Companion (silica gel, heptane/0-20% gradient of ethyl acetate). The isolated product was recrystallized from 100 ml of ethanol to give 7.80 g (39% yield) of Intermediate 39-1 as a white solid.

¹H NMR (400 MHz, CD₂Cl₂) δ 8.17 (dt, 2H), 7.54-7.25 (m, 11H), 7.19 (ddd, 1H), 7.12 (t, 1H), 7.09-7.02 (m, 4H), 7.02-6.88 (m, 3H), 6.82 (d, 1H), 6.76 (d, 1H), 3.32-3.15 (m, 2H), 3.01-2.84 (q, 2H), 2.21 (s, 3H), 2.04-1.85 (d, 7H), 1.34 (s, 9H).

### Compound 39

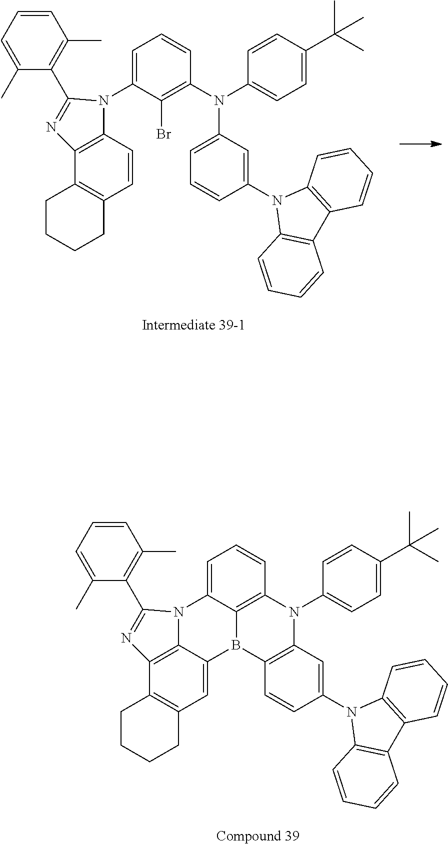 [SMILES: Cc1cccc(C)c1-c1nc2c3c(ccc2n1-c1cccc(N(c2ccc(C(C)(C)C)cc2)c2cccc(-n4c5ccccc5c5ccccc54)c2)c1Br)CCCC3 ; Cc1cccc(C)c1-c1nc2c3c(cc4c2n1-c1cccc2c1B4c1ccc(-n4c5ccccc5c5ccccc54)cc1N2c1ccc(C(C)(C)C)cc1)CCCC3]

6.50 g (7.93 mmol) of Intermediate 39-1 were dissolved in 80 ml of water-free tert-butylbenzene. 8.35 ml of tert-butyllithium (1.9 M in pentane) were slowly added at −3° C. and stirred up to a temperature of 3° C. during 25 minutes. The solution was cooled down to −49° C. and 1.50 ml (15.9 mmol) of tribromoborane were added. The yellow suspension was warmed up to 15° C. during 15 minutes and cooled down to −2° C. 2.77 ml (15.9 mmol) of N,N-diisopropylethylamine were slowly added and the yellow suspension heated at 156° C. during 21 hours. The orange suspension was cooled down and treated with 50 ml of 10% aqueous sodium acetate solution and 50 ml of heptane. The suspension was filtered and the solid washed with 100 ml of water and 100 ml of heptane. The yellow solid was further purified by MPLC with the CombiFlash Companion (silica gel, dichloromethane/0-70% gradient of ethyl acetate). The isolated product was suspended in 30 ml of ethanol, filtered and washed with ethanol. The solid was dissolved in 40 ml of dichloromethane and mixed with 40 ml of ethanol. The solution was concentrated under vacuum to a volume of 40 ml until a suspension formed. The suspension was filtered and the solid washed with ethanol to give 1.01 g (17% yield) of Compound 39 as a yellow solid.

ESI-MS (positive, m/z): exact mass of C₅₃H₄₅BN₄=748.37; found 749.7 [M+1]⁺.

### Compound 40

### Intermediate 40-1

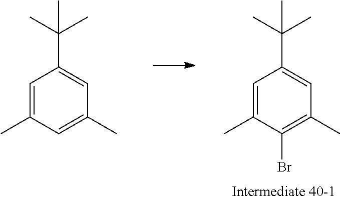

100 g (0.62 mol) of 1-(tert-butyl)-3,5-dimethylbenzene were dissolved in 700 ml of acetic acid. 89.0 g (0.56 mol) of bromine in 300 ml of acetic acid were slowly added at a maximum temperature of 10° C. and stirring continued up to a temperature of 17° C. during 3 hours. Stirring was continued at room temperature during 17 hours. 300 ml of 3% aqueous solution of sodium thiosulfate were dropwise added and the resulting reaction mixture stirred during 30 minutes, followed by stirring at 5° C. The reaction mixture was filtered and the solid dissolved in 400 ml of dichloromethane. The solution was treated with 200 ml of water and 200 ml of saturated aqueous sodium bicarbonate solution, followed by stirring during 5 minutes. The organic phase was washed with 300 ml of water and 200 ml of saturated aqueous sodium chloride solution. The organic phase was dried over magnesium sulfate and concentrated under vacuum. The product was heated in 500 ml of methanol until a clear solution formed. The solution was cooled down to room temperature and stirred until a suspension formed. The suspension was filtered to give 67.0 g of a white solid. An additional amount of solid was isolated from the filtrated by repeating the same crystallization procedure, giving a total of 101 g (75% yield) of Intermediate 40-1 as a white solid.

¹H NMR (300 MHz, CD₂Cl₂) δ 7.15 (s, 2H), 2.44 (s, 6H), 1.33 (s, 9H).

### Intermediate 40-2

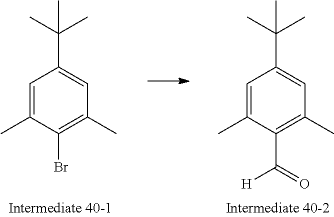

55.5 g (0.23 mol) of Intermediate 40-1 were dissolved in 500 ml of tetrahydrofuran and cooled down to −78° C. 100 ml of n-butyllithium (2.5 M in hexanes) were slowly added, followed by slow addition of 21 ml (0.27 mol) of N,N-dimethylformamide at a maximum temperature of −50° C. The solution was warmed up to −5° C. during 30 minutes and treated with 30 ml of water. The reaction mixture was diluted with 1000 ml of water and 300 ml of ethyl acetate and stirred during 10 minutes. The organic phase was removed and the aqueous phase washed with ethyl acetate (2×200 ml). The combined organic phases were washed with 300 ml of saturated aqueous sodium chloride solution, dried over sodium sulfate and concentrated under vacuum. The product was further purified by fractional distillation to give 37.3 g (86% yield) of Intermediate 40-2 as a white solid.

¹H NMR (300 MHz, CD₂Cl₂) δ 10.61 (s, 1H), 7.16 (s, 2H), 2.64 (s, 6H), 1.36 (s, 9H).

### Intermediate 40-3

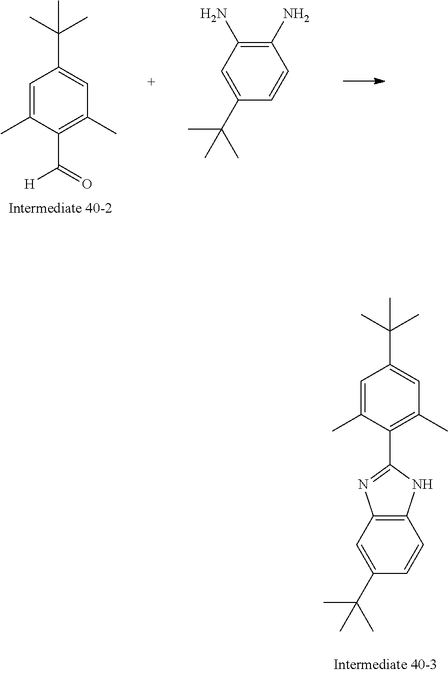

12.9 g (79 mmol) of 4-(tert-butyl)benzene-1,2-diamine and 8.30 g (79 mmol) of sodium bisulfite in 150 ml of N,N-dimethylacetamide were heated to 114° C. 15.2 g (80 mmol) of Intermediate 40-2 were added and heating continued at 114° C. during 3 hours. The reaction mixture was poured into 800 ml of water, and the resulting suspension filtered. The solid was dissolved in 1000 ml of dichloromethane and washed with 100 ml of water and 100 ml of saturated aqueous sodium chloride solution. The organic phase was dried over sodium sulfate and filtered over a 12 cm layer of silica gel. The silica gel was rinsed with plenty of dichloromethane and ethyl acetate. The collected eluents were concentrated under vacuum, and the solid dissolved in 800 ml of ethyl acetate. The solution was concentrated to a volume of 350 ml and stirred at room temperature until a suspension formed. The suspension was further stirred during 3 hours, then filtered and the solid washed with a small amount of ethyl acetate to give 17.3 g (65% yield) of Intermediate 40-3 as a solid.

¹H NMR (300 MHz, DMSO-d₆) δ 12.29 (s, 1H), 7.72-7.23 (m, 3H), 7.20 (s, 2H), 2.10 (s, 6H), 1.37 (s, 9H), 1.32 (s, 9H).

### Intermediate 40-4

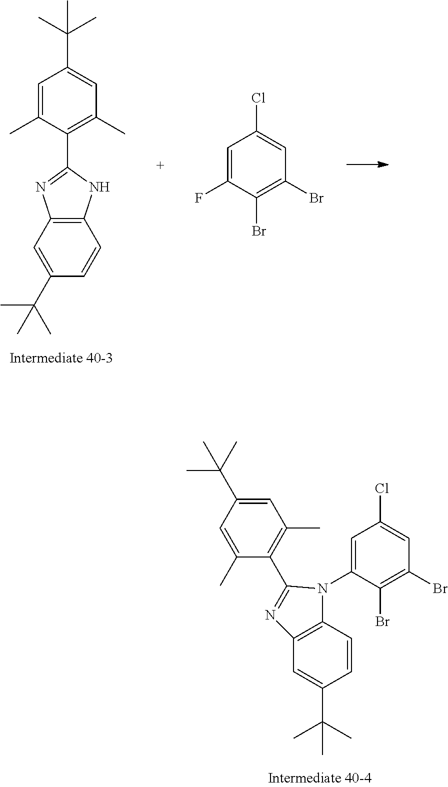 [SMILES: Cc1cc(C(C)(C)C)cc(C)c1-c1nc2cc(C(C)(C)C)ccc2[nH]1 ; Fc1cc(Cl)cc(Br)c1Br ; Cc1cc(C(C)(C)C)cc(C)c1-c1nc2cc(C(C)(C)C)ccc2n1-c1cc(Cl)cc(Br)c1Br]

44.0 g (132 mmol) of Intermediate 40-3 and 42.0 g (145 mmol) of 1,2-dibromo-5-chloro-3-fluorobenzene and 140 g (0.66 mol) of potassium phosphate were suspended in 500 ml of N,N-dimethylformamide, followed by heating at 98° C. during 1 hour. The reaction mixture was cooled down to room temperature and poured into 1500 ml of water. The suspension was filtered and the solid washed with water. The solid was dissolved in heptane and the solution dried over sodium sulfate, filtered and eluted over a small layer of silica gel. The solution was concentrated under vacuum. The product was purified by MPLC in with the CombiFlash Companion (silica gel, heptane/tert-butyl methyl ether). The product fractions eluting first were concentrated under vacuum. The product was mixed with hot 2-propanol and stirred until precipitation started. The suspension was further stirred at room temperature and filtered, The solid was washed with cold 2-propanol to give 16.5 g (21% yield) of Intermediate 40-4 as a white solid.

¹H NMR (300 MHz, CD₂Cl₂) δ 7.92 (d, 1H), 7.72 (d, 1H), 7.45 (dd, 1H), 7.18 (br. s, 1H), 7.10 (dd, 1H), 7.07-7.01 (m, 2H), 2.26 (s, 3H), 2.10 (s, 3H), 1.48 (s, 9H), 1.33 (s, 9H).

### Intermediate 40-5

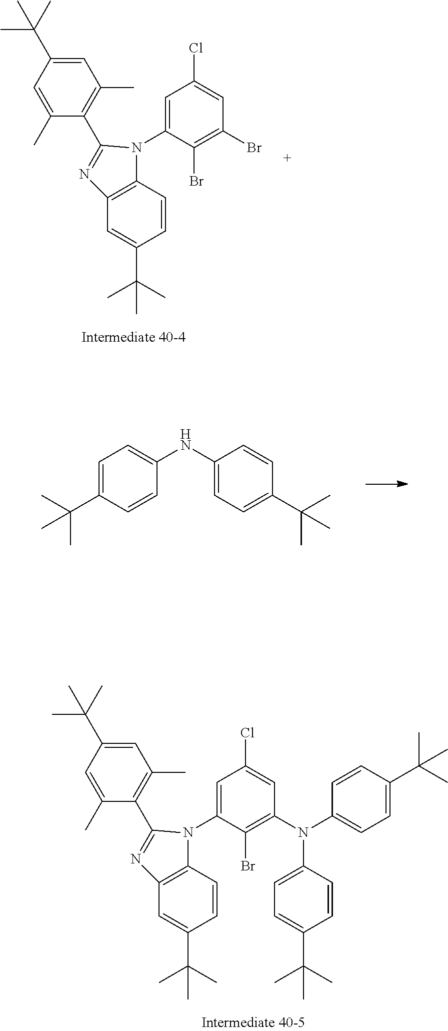

16.1 g (26.7 mmol) of Intermediate 40-4, 9.40 g (33.2 mmol) of bis(4-(tert-butyl)phenyl)amine, 0.49 g (0.53 mmol) of tris(dibenzylideneacetone)dipalladium(0), 0.62 g (2.12 mmol) of tri-tert-butylphosphonium tetrafluoroborate, and 6.50 g (67.6 mmol) of sodium tert-butoxide were suspended in 160 ml of toluene. The dark suspension was three times evacuated and backfilled with argon and heated at 75° C. during 19 hours. The reaction mixture was diluted with 100 ml of toluene and 50 ml of water. The organic phase was separated and washed with 100 ml of water and 100 ml of saturated aqueous sodium chloride solution, followed by drying over sodium sulfate, and concentration under vacuum. The product was purified by MPLC with the CombiFlash Companion (silica gel, toluene/ethyl acetate). The isolated product was dissolved in 100 ml of hot ethanol and stirred at room temperature until a suspension formed. Stirring was continued at ice bath temperature. The suspension was filtered and the solid washed with a small amount of methanol to give 14.8 g (69% yield) of Intermediate 40-5 as a white solid.

¹H NMR (300 MHz, CD₂Cl₂) δ 7.90 (d, 1H), 7.43 (dd, 1H), 7.39-7.27 (m, 4H), 7.26-7.03 (m, 4H), 6.98 (d, 1H), 6.93-6.76 (m, 4H), 2.26 (s, 3H), 2.08 (s, 3H), 1.47 (s, 9H), 1.39 (s, 9H), 1.37 (s, 18H).

### Intermediate 40-6

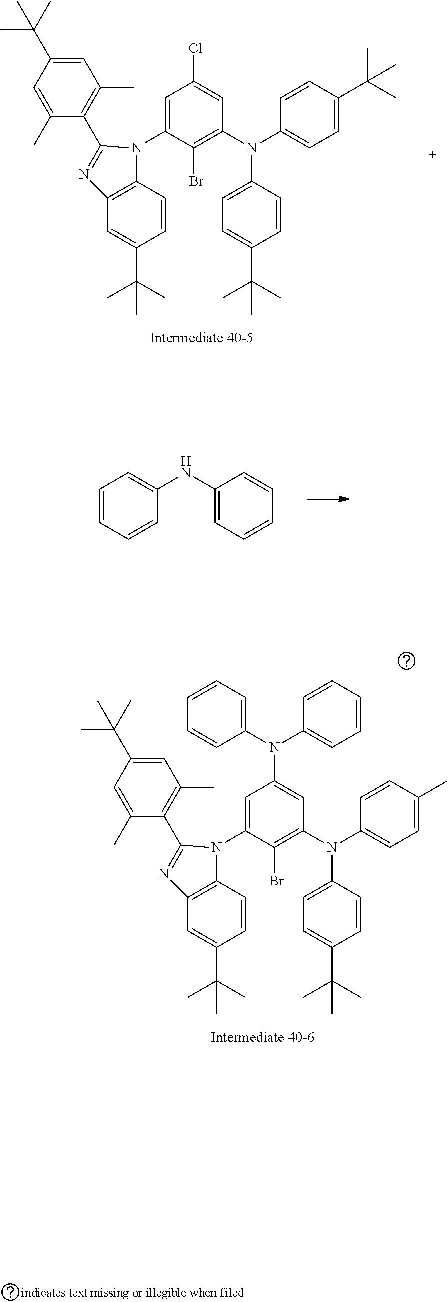

12.9 g (16.1 mmol) of Intermediate 40-5, 3.5 g (20.7 mmol) of diphenylamine, 0.31 g (0.33 mmol) of tris(dibenzylideneacetone)dipalladium(0), 0.39 g (1.34 mmol) of tri-tert-butylphosphonium tetrafluoroborate, and 3.9 g (40 mmol) of sodium tert-butoxide were suspended in 130 ml of o-xylene. The dark suspension was three times evacuated and backfilled with argon and heated at 100° C. during 23 hours. 245 mg (0.27 mmol) of tris(dibenzylideneacetone)dipalladium(0) and 311 mg (1.07 mmol) of tri-tert-butylphosphonium tetrafluoroborate were added and heating continued at 108° C. during 21 hours. 0.31 g of tris(dibenzylideneacetone)dipalladium(0), and 0.39 g of tri-tert-butylphosphonium tetrafluoroborate were added and stirring continued at 100° C. during 19 hours. The dark suspension was cooled down and 50 ml of toluene and 50 ml of water were added. The organic phase was separated and washed with 100 ml of water and 50 ml of saturated aqueous sodium chloride solution, followed by drying over sodium sulfate. The orange solution was filtered over a 3 cm layer of silica gel followed by rinsing the silica gel layer with toluene. The collected eluents were concentrated under vacuum. The product was mixed with 100 ml of ethanol and heated at 60° C. The solution was cooled down to room temperature and stirring continued during 2 hours. The suspension was filtered and the solid washed with a small amount of methanol to give 5.2 g (34% yield) of Intermediate 40-6 as a white solid.

¹H NMR (300 MHz, CD₂Cl₂) δ 7.84 (br. s, 1H), 7.42 (dd, 1H), 7.35-7.04 (m, 13H), 6.94-6.80 (m, 9H), 6.40 (d, 1H), 2.06 (s, 3H), 1.74 (s, 3H), 1.45 (s, 18H), 1.36 (s, 18H).

### Compound 40

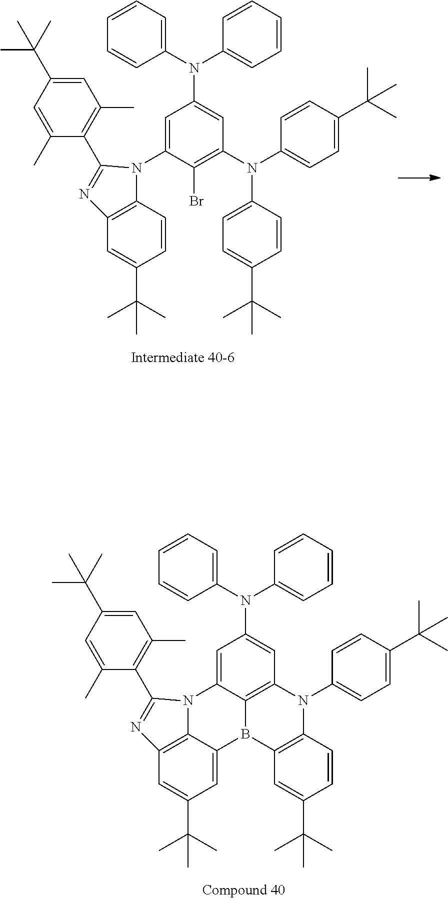

7.50 g (8.01 mmol) of Intermediate 40-6 were dissolved in 100 ml of water-free tert-butylbenzene and heated at 60° C. until a solution formed. 8.5 ml of tert-butyllithium (1.9 M in pentane) were slowly added at −30° C. and stirred up to room temperature during 30 minutes. The solution was cooled down to −6° C. and 1.55 ml (16 mmol) of tribromoborane were added. The yellow suspension was warmed up to 21° C. during 15 minutes and cooled down to −11° C. 2.8 ml (16 mmol) of N,N-diisopropylethylamine were slowly added and the yellow suspension heated at 163° C. during 47 hours. During the whole heating period the reaction mixture was feeded five times with 2.8 ml of N,N-diisopropylethylamine. The dark reaction mixture was cooled down and treated with 75 ml of 10% aqueous sodium acetate solution followed by stirring at 10° C. during 1 hour. The suspension was filtered and the solid washed with 50 ml of water and 50 ml of heptane. The yellow solid was further purified by MPLC with the CombiFlash Companion (silica gel, dichloromethane). The isolated product was heated in 100 ml of ethyl acetate until a solution formed. The solution was concentrated to a volume of 20 ml and the resulting suspension stirred at room temperature during 20 minutes. The suspension was filtered and the solid washed with a small amount of ethyl acetate to give 1.1 g (16% yield) of Compound 40 as a white solid.

¹H NMR (300 MHz, CD₂Cl₂) δ 9.13 (d, 1H), 8.95 (d, 1H), 8.13 (d, 1H), 7.58 (dd, 1H), 7.50 (d, 2H), 7.25-6.67 (m, 16H), 5.92 (d, 1H), 2.03 (s, 6H), 1.66 (s, 9H), 1.58 (s, 9H), 1.37 (s, 9H), 1.31 (s, 9H).

### Compound 41

### Intermediate 41-1

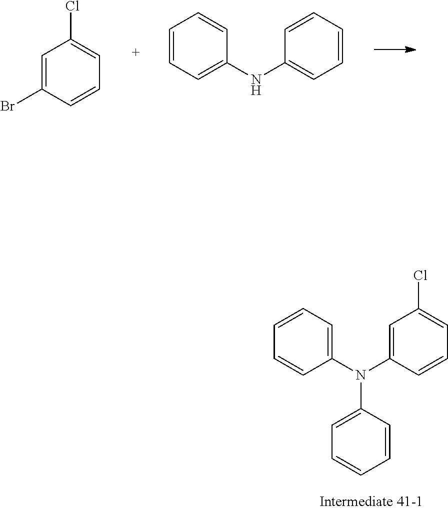 [SMILES: Clc1cccc(Br)c1 ; c1ccc(Nc2ccccc2)cc1 ; Clc1cccc(N(c2ccccc2)c2ccccc2)c1]

23.0 g (120 mmol) of 1-bromo-3-chlorobenzene, 20.3 g (120 mmol) of diphenylamine, 549 mg (0.60 mmol) of tris(dibenzylideneacetone)dipalladium(0), 696 mg (2.40 mmol) of tri-tert-butylphosphonium tetrafluoroborate, and 16.1 g (168 mmol) of sodium tert-butoxide were suspended in 200 ml of toluene. The dark suspension was three times evacuated and backfilled with argon and heated at 102° C. during 20 minutes. The reaction mixture was cooled down, 100 ml of water and 1 g of sodium cyanide were added, and the resulting mixture stirred during 1 hour. The organic phase was separated and extracted with water (3×100 ml), dried over sodium sulfate and concentrated under vacuum. The resulting solid was recrystallized from ethanol to give 23.3 g (70% yield) of Intermediate 41-1.

¹H NMR (300 MHz, CDCl₃) δ 7.35-7.25 (m, 4H), 7.19-7.03 (m, 8H), 6.99-6.91 (2 m, 2H).

### Intermediate 41-2

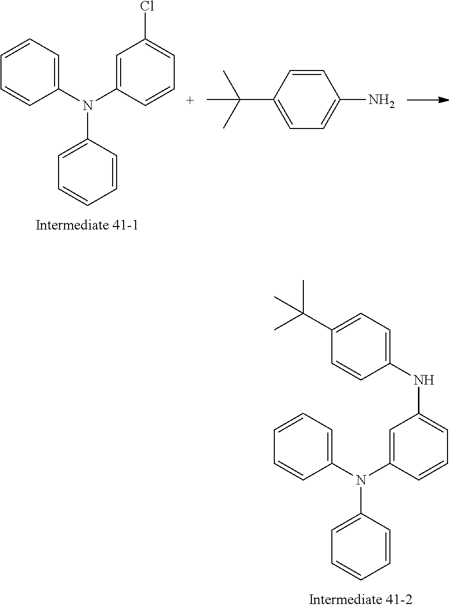

23.0 g (82 mmol) of Intermediate 41-1, 13.5 g (90 mmol) of 4-(tert-butyl)aniline, 1.51 g (1.64 mmol) of tris(dibenzylideneacetone)dipalladium(0), 1.91 g (6.58 mmol) of tri-tert-butylphosphonium tetrafluoroborate, and 19.8 g (206 mmol) of sodium tert-butoxide were suspended in 200 ml of toluene. The dark suspension was three times evacuated and backfilled with argon and heated at 108° C. during 23 hours. The reaction mixture was cooled down, 100 ml of water and 1 g of sodium cyanide were added, and the resulting mixture stirred during 1 hour. The organic phase was separated and extracted with water (3×100 ml), dried over sodium sulfate and concentrated under vacuum. The solid was further purified by MPLC with the CombiFlash Companion (silica gel, heptane/0-4% gradient of ethyl acetate) to give 31.8 g (98% yield) of Intermediate 41-2.

¹H NMR (300 MHz, CD₂Cl₂) δ 7.36-7.24 (m, 6H), 7.21-7.11 (m, 5H), 7.11-6.93 (m, 4H), 6.79-6.71 (m, 2H), 6.65-6.57 (m, 1H), 5.72 (br. s, 1H), 1.34 (s, 9H).

### Intermediate 41-3

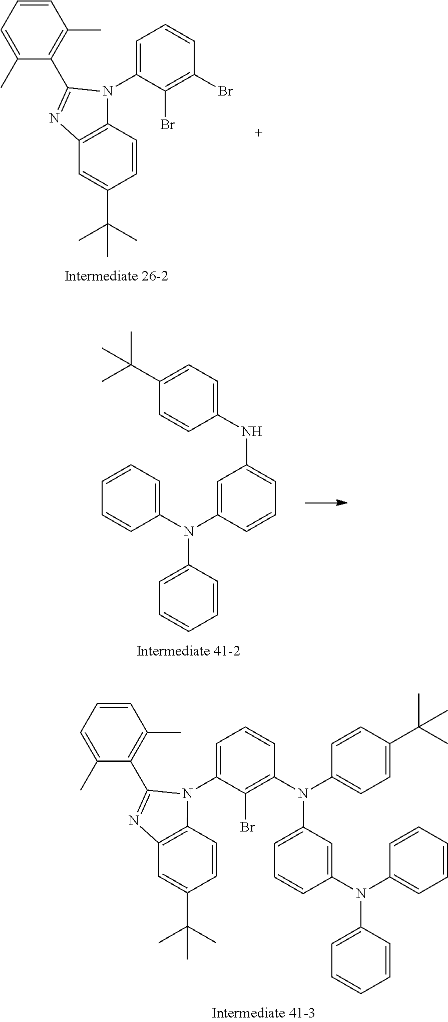 [SMILES: Cc1cccc(C)c1-c1nc2cc(C(C)(C)C)ccc2n1-c1cccc(Br)c1Br ; CC(C)(C)c1ccc(Nc2cccc(N(c3ccccc3)c3ccccc3)c2)cc1 ; Cc1cccc(C)c1-c1nc2cc(C(C)(C)C)ccc2n1-c1cccc(N(c2ccc(C(C)(C)C)cc2)c2cccc(N(c3ccccc3)c3ccccc3)c2)c1Br]

9.50 g (18.5 mmol) of Intermediate 26-2, 7.70 g (19.6 mmol) of Intermediate 41-2, 334 mg (0.37 mmol) of tris(dibenzylideneacetone)dipalladium(0), 430 mg (1.48 mmol) of tri-ter-butylphosphonium tetrafluoroborate, and 4.46 g (46.4 mmol) of sodium tert-butoxide were suspended in 100 ml of toluene. The dark suspension was three times evacuated and backfilled with argon and heated at 102° C. during 25 minutes. The reaction mixture was cooled down, 100 ml of water and 1 g of sodium cyanide were added, and the resulting mixture stirred during 1 hour. The organic phase was separated and extracted with water (3×100 ml), dried over sodium sulfate and concentrated under vacuum. The solid was further purified by MPLC with the CombiFlash Companion (silica gel, heptane/0-60% gradient of dichloromethane) to give 10.7 g (70% yield) of Intermediate 41-3.

¹H NMR (300 MHz, CD₂Cl₂) δ 7.91 (d, 1H), 7.40 (dd, 1H), 7.33-7.19 (m, 9H), 7.19-7.06 (m, 6H), 7.06-6.92 (m, 5H), 6.89-6.78 (m, 2H), 6.76-6.62 (m, 2H), 6.46 (d, 1H), 2.25 (s, 3H), 2.07 (s, 3H), 1.47 (s 9H), 1.33 (s, 9H).

### Compound 41

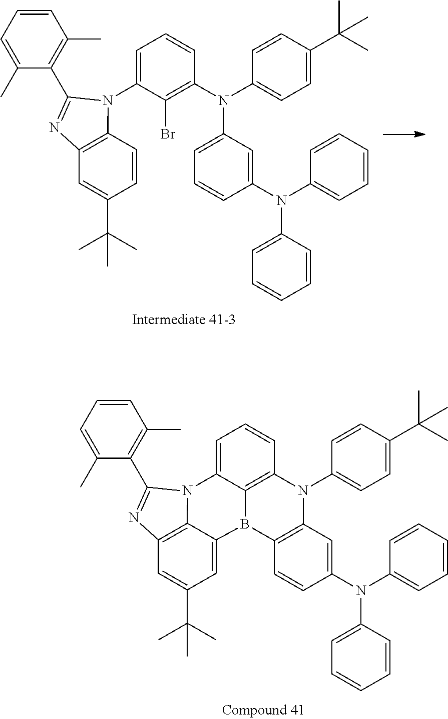 [SMILES: Cc1cccc(C)c1-c1nc2cc(C(C)(C)C)ccc2n1-c1cccc(N(c2ccc(C(C)(C)C)cc2)c2cccc(N(c3ccccc3)c3ccccc3)c2)c1Br ; Cc1cccc(C)c1-c1nc2cc(C(C)(C)C)cc3c2n1-c1cccc2c1B3c1ccc(N(c3ccccc3)c3ccccc3)cc1N2c1ccc(C(C)(C)C)cc1]

10.0 g (12.1 mmol) of Intermediate 41-3 were dissolved in 149 ml of water-free tert-butylbenzene. 12.8 ml of tert-butyllithium (1.9 M in pentane) were slowly added at −24° C. and stirred up to a temperature of 5° C. during 15 minutes. The solution was cooled down to −51° C. and 2.3 ml (24 mmol) of tribromoborane were added. The yellow suspension was warmed up to 13° C. during 15 minutes and cooled down to −6° C. 4.24 ml (24 mmol) of N,N-diiso-propylethylamine were slowly added and the yellow suspension heated at 156° C. The reaction mixture was feeded four times with an additional amount of 4.24 ml of N,N-diiso-propylethylamine over a total reaction time of 25 hours. The orange suspension was cooled down and treated with 10 ml of 10% aqueous sodium acetate solution and 300 ml of heptane, and the mixture stirred during 1 hour at room temperature. The organic phase was separated and washed with water (3×200 ml), dried over sodium sulfate and concentrated under vacuum. The product was further purified by MPLC with the CombiFlash Companion (silica gel, dichloromethane/0-10% gradient of ethyl acetate). The product fractions were combined and concentrated under vacuum. The solid was dissolved in 30 ml of dichloromethane and 50 ml of ethyl acetate. The solution was concentrated under vacuum until a suspension formed. The suspension was filtered and the solid washed with ethyl acetate to give 0.75 g of Compound 41 as a yellow solid. The filtrate was left standing overnight. The resulting suspension was filtered and the solid washed with ethyl acetate to give an additional 0.30 g of Compound 41 for a total yield of 11%.

¹H NMR (300 MHz, CD₂Cl₂) δ 8.89 (d, 1H), 8.82 (d, 1H), 8.17 (d, 1H), 7.59-7.40 (m, 3H), 7.37-7.24 (m, 6H), 7.22-7.06 (m, 9H), 7.01 (dd, 1H), 6.62 (d, 1H), 6.52 (d, 1H), 6.21 (d, 1H), 2.15 (s, 6H), 1.62 (s, 9H), 1.38 (s, 9H).

### Compound 42

### Intermediate 42-1

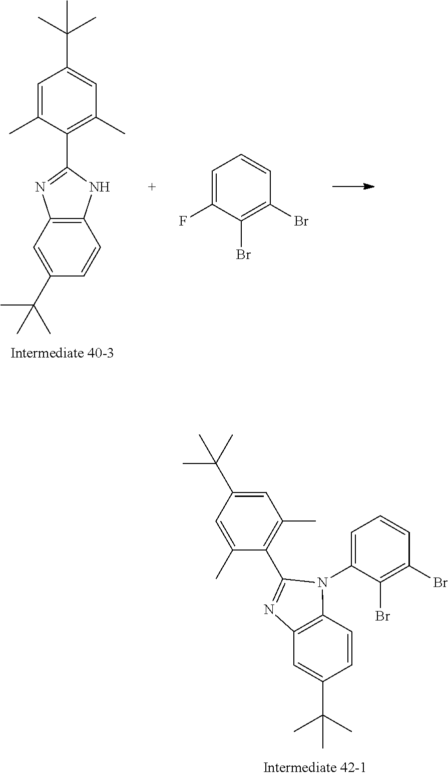 [SMILES: Cc1cc(C(C)(C)C)cc(C)c1-c1nc2cc(C(C)(C)C)ccc2[nH]1 ; Fc1cccc(Br)c1Br ; Cc1cc(C(C)(C)C)cc(C)c1-c1nc2cc(C(C)(C)C)ccc2n1-c1cccc(Br)c1Br]

25.0 g (74.7 mmol) of Intermediate 40-3, 21.2 g (83.5 mmol) of 2,3-dibromofluorobenzene and 64.7 g (0.30 mol) of potassium phosphate were suspended in 250 ml of N,N-dimethylformamide, followed by heating at 137° C. during 2 hours. The reaction mixture was cooled down to room temperature and poured into 1500 ml of water. The suspension was filtered and the solid washed with water. The solid was dissolved in 500 ml of dichloromethane and washed with saturated aqueous sodium chloride solution. The organic phase was separated, then dried over sodium sulfate, filtered and concentrated under vacuum. The resulting solid was further purified by MPLC with the CombiFlash Companion (silica gel, dichloromethane). The product fractions were collected and concentrated under vacuum to give 17.2 g (41% yield) of Intermediate 42-1 as a white solid.

¹H NMR (300 MHz, CD₂Cl₂) δ 7.91 (d, 1H), 7.71 (dd, 1H), 7.44 (dd, 1H), 7.23-6.99 (m, 5H), 2.26 (s, 3H), 2.12 (s, 3H), 1.48 (s, 9H), 1.32 (s, 9H).

### Intermediate 42-2

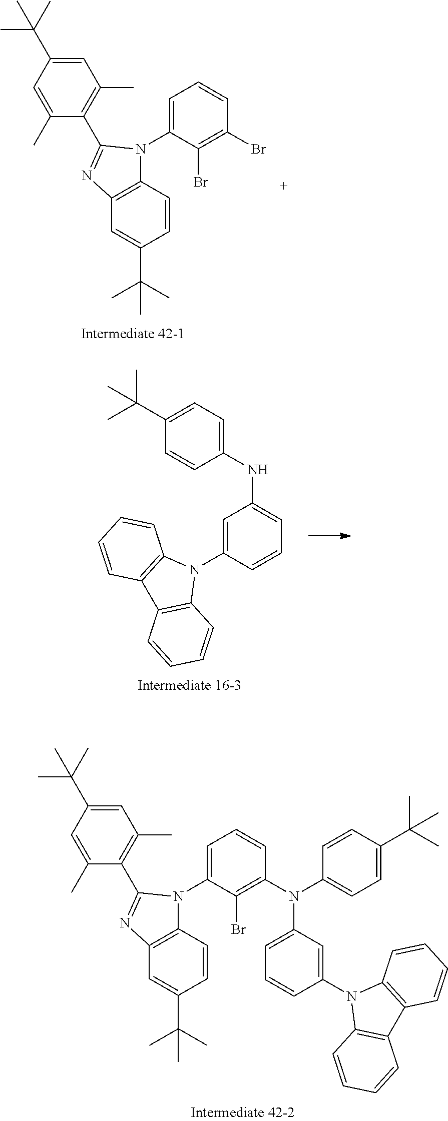 [SMILES: Cc1cc(C(C)(C)C)cc(C)c1-c1nc2cc(C(C)(C)C)ccc2n1-c1cccc(Br)c1Br ; CC(C)(C)c1ccc(Nc2cccc(-n3c4ccccc4c4ccccc43)c2)cc1 ; Cc1cc(C(C)(C)C)cc(C)c1-c1nc2cc(C(C)(C)C)ccc2n1-c1cccc(N(c2ccc(C(C)(C)C)cc2)c2cccc(-n3c4ccccc4c4ccccc43)c2)c1Br]

9.10 g (16.0 mmol) of Intermediate 42-1, 6.70 g (17.2 mmol) of Intermediate 16-3, 299 mg (0.32 mmol) of tris(dibenzylideneacetone)dipalladium(0), 371 mg (1.27 mmol) of tri-tert-butylphosphonium tetrafluoroborate, and 3.80 g (39.6 mmol) of sodium tert-butoxide were suspended in 100 ml of o-xylene. The dark suspension was three times evacuated and backfilled with argon and heated at 101° C. during 6 hours. The reaction mixture was treated with 100 ml of water and 0.3 g of sodium cyanide and heated under reflux during 1 hour. The organic phase was separated and extracted with 100 ml of water and 100 ml of saturated aqueous sodium chloride solution, then dried over sodium sulfate and filtered over a 12 cm layer of silica gel. The silica gel layer was rinsed with dichloromethane and the combined eluents concentrated under vacuum. The product was purified by MPLC with the CombiFlash Companion (silica gel, dichloromethane). The product fractions were combined and concentrated under vacuum. The resulting oil was left at room temperature until a solid formed. The solid was suspended in ethanol under reflux during 90 minutes and further stirred at room temperature during 30 minutes. The suspension was filtered and the solid washed with ethanol to give 8.5 g (61% yield) of Intermediate 42-2 as a white solid.

¹H NMR (300 MHz, CD₂Cl₂) δ 8.17 (d, 2H), 7.89 (br. s, 1H), 7.58-7.23 (m, 12H), 7.23-7.17 (m, 1H), 7.16-7.04 (m, 4H), 7.03-6.92 (ddd, 3H), 6.90 (br. s, 1H), 2.25 (s, 3H), 1.99 (s, 3H), 1.46 (s, 9H), 1.35 (s, 9H), 1.31 (s, 9H).

### Compound 42

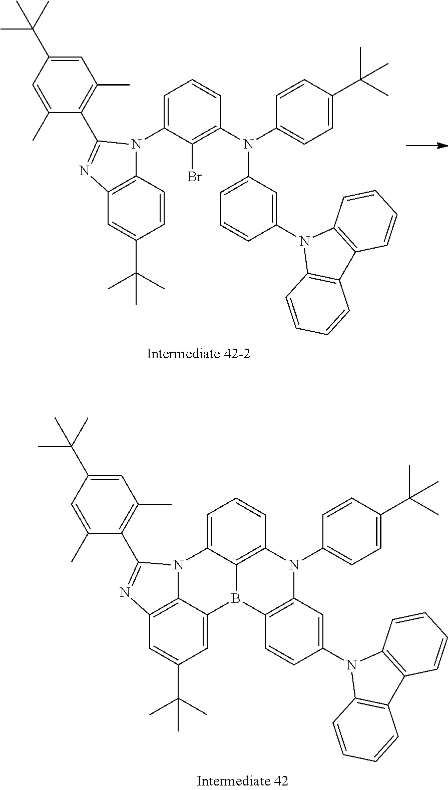 [SMILES: Cc1cc(C(C)(C)C)cc(C)c1-c1nc2cc(C(C)(C)C)ccc2n1-c1cccc(N(c2ccc(C(C)(C)C)cc2)c2cccc(-n3c4ccccc4c4ccccc43)c2)c1Br ; Cc1cc(C(C)(C)C)cc(C)c1-c1nc2cc(C(C)(C)C)cc3c2n1-c1cccc2c1B3c1ccc(-n3c4ccccc4c4ccccc43)cc1N2c1ccc(C(C)(C)C)cc1]

8.50 g (9.68 mmol) of Intermediate 42-2 were dissolved in 85 ml of water-free tert-butylbenzene. 10.2 ml of tert-butyllithium (1.9 M in pentane) were slowly added at −30° C. and stirred up to a temperature of 10° C. during 30 minutes. The solution was cooled down to −30° C. and 1.85 ml (19.4 mmol) of tribromoborane were added. The orange suspension was warmed up to 18° C. during 15 minutes and cooled down to −30° C. 3.4 ml (19.4 mmol) of N,N-diisopropylethylamine were slowly added and the yellow suspension heated up to 138° C. during 30 minutes. 3.4 ml of N,N-diisopropylethylamine were added and heating continued at 159° C. during 40 hours. The reaction mixture was feeded three times with 3.4 ml of N,N-diisopropylethylamine during the whole reaction time after regular intervals. The dark reaction mixture was cooled down to 90° C., treated with 100 ml of 10% aqueous sodium acetate, and diluted with 100 ml of heptane. The organic phase was washed with water (3×100 ml), then with 50 ml of saturated aqueous sodium chloride, followed by concentration under vacuum. The orange oil was further purified by MPLC with the CombiFlash Companion (silica gel, dichloromethane/0-10% gradient of ethyl acetate). The isolated product was stirred in ethanol until a suspension formed. The suspension was filtered and the solid washed with ethanol. An additional amount of solid precipitated out from the filtrate. The isolated solids were combined and dissolved in a mixture of 100 ml of dichloromethane and 100 ml of ethyl acetate. The solution was concentrated until a suspension formed. The suspension was filtered and the solid washed with a small amount of ethyl acetate to give 1.34 g (17% yield) of Compound 42 as a yellow solid.

¹H NMR (300 MHz, CD₂Cl₂) δ 9.34 (d, 1H), 9.00 (s, 1H), 8.27 (s, 1H), 8.15 (d, 2H), 7.81-7.64 (m, 3H), 7.57 (d, 2H), 7.50-7.22 (m, 9H), 7.06 (d, 1H), 6.80 (d, 1H), 6.68 (d, 1H), 2.18 (s, 6H), 1.69 (s, 9H), 1.47 (s, 9H), 1.38 (s, 9H).

### Compound 43

### Intermediate 43-1

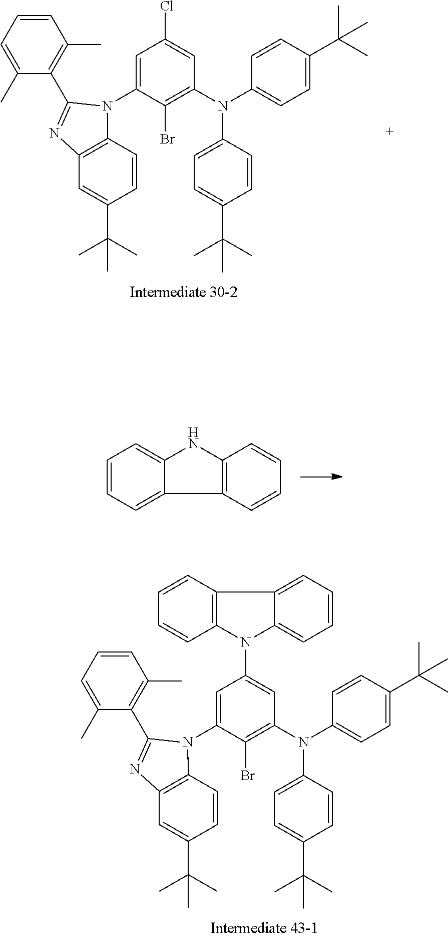 [SMILES: Cc1cccc(C)c1-c1nc2cc(C(C)(C)C)ccc2n1-c1cc(Cl)cc(N(c2ccc(C(C)(C)C)cc2)c2ccc(C(C)(C)C)cc2)c1Br ; c1ccc2c(c1)[nH]c1ccccc12 ; Cc1cccc(C)c1-c1nc2cc(C(C)(C)C)ccc2n1-c1cc(-n2c3ccccc3c3ccccc32)cc(N(c2ccc(C(C)(C)C)cc2)c2ccc(C(C)(C)C)cc2)c1Br]

10.0 g (13.4 mmol) of Intermediate 30-2, 3.36 g (20.0 mmol) of carbazole, 245 mg (0.27 mmol) of tris(dibenzylideneacetone)dipalladium(0), 311 mg (1.07 mmol) of tri-tert-butylphosphonium tetrafluoroborate, and 3.22 g (33.5 mmol) of sodium tert-butoxide were suspended in 100 ml of o-xylene. The dark suspension was three times evacuated and backfilled with argon and heated at 122° C. during 24 hours. 245 mg (0.27 mmol) of tris(dibenzylideneacetone)dipalladium(0) and 311 mg (1.07 mmol) of tri-tert-butylphosphonium tetrafluoroborate were added and heating continued at 122° C. during 48 hours. The reaction mixture was cooled down, 100 ml of water and 1 g of sodium cyanide were added, and the resulting mixture stirred during 1 hour. The organic phase was separated and extracted with water (3×100 ml), dried over sodium sulfate and concentrated under vacuum. The solid was further purified by MPLC with the CombiFlash Companion (silica gel, heptane/0-50% gradient of dichloromethane). The isolated product was recrystallized from 100 ml of ethanol and further purified by MPLC (silica gel, heptane/0-20% gradient of ethyl acetate) to give 8.3 g (71% yield) of Intermediate 43-1.

ESI-MS (positive, m/z): exact mass of C₅₇H₅₇BrN₄=876.38; found 877.4 [M+1]⁺.

### Compound 43

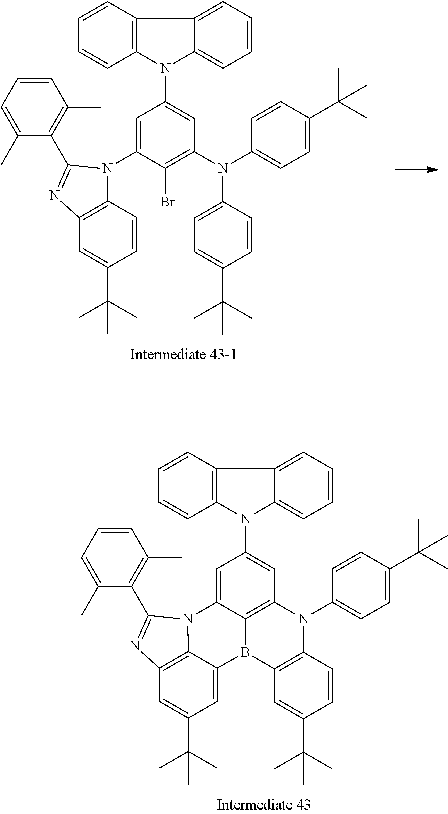 [SMILES: Cc1cccc(C)c1-c1nc2cc(C(C)(C)C)ccc2n1-c1cc(-n2c3ccccc3c3ccccc32)cc(N(c2ccc(C(C)(C)C)cc2)c2ccc(C(C)(C)C)cc2)c1Br ; Cc1cccc(C)c1-c1nc2cc(C(C)(C)C)cc3c2n1-c1cc(-n2c4ccccc4c4ccccc42)cc2c1B3c1cc(C(C)(C)C)ccc1N2c1ccc(C(C)(C)C)cc1]

6.00 g (6.83 mmol) of Intermediate 43-1 were dissolved in 84 ml of water-free tert-butylbenzene. 7.19 ml of tert-butyllithium (1.9 M in pentane) were slowly added at −27° C. and stirred up to a temperature of −4° C. during 20 minutes. The solution was cooled down to −51° C. and 1.3 ml (13.6 mmol) of tribromoborane were added. The yellow suspension was warmed up to 8° C. during 10 minutes and cooled down to −3° C. 2.4 ml (13.6 mmol) of N,N-diisopropylethylamine were slowly added and the yellow suspension heated at 164° C. during 48 hours. During the whole heating period, five times an additional amount of each 2.4 ml of N,N-diisopropylethylamine was added in one shot in regular intervals. The orange suspension was cooled down and treated with 50 ml of 10% aqueous sodium acetate solution and 100 ml of heptane. The organic phase was separated and washed with water (3×50 ml), dried over sodium sulfate and concentrated under vacuum. The yellow solid was further purified by MPLC with the CombiFlash Companion (silica gel, dichloromethane/0-10% gradient of ethyl acetate). The product was subjected to an additional MPLC purification (silica gel, dichloromethane/0-10% gradient of ethyl acetate). The product fractions were concentrated under vacuum, diluted with 20 ml of hexane and stirred during 30 minutes. The suspension was filtered to give 150 mg (2.7% yield) of Compound 43 as a yellow solid.

ESI-MS (positive, m/z): exact mass of C₅₇H₅₅BN₄=806.45; found 807.4 [M+1]⁺.

### Compound 44

### Intermediate 44-1

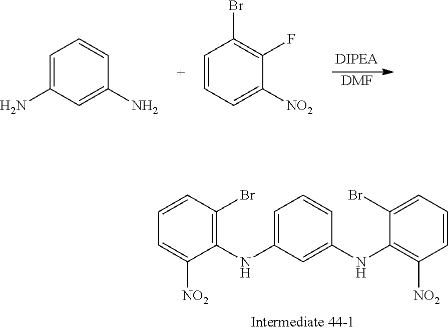

4.0 g (37.0 mmol) of 1,3-diaminobenzene, 17.9 g (81.0 mmol) of 1-bromo-2-fluoro-3-nitrobenzene, and 10.52 g (81 mmol) of N,N-diisopropylethylamine were dissolved in 37 ml of N,N-dimethylformamide. The reaction mixture was heated to 120° C. for 50 hours, then cooled to room temperature and poured into water. The pH was adjusted to pH 1, and the crude product was extracted with dichloromethane, and organic phases were combined. The solvent was removed on the rotavap, and the crude product was recrystallized from ethylacetate/ethanol/water (2/2/1) and filtered to give 13.98 g (74% yield) of Intermediate 44-1 as a red powder.

¹H NMR (300 MHz, DMSO-d₆) δ 8.07 (s, 2H), 8.01 (dd, J=8.0, 1.5 Hz, 2H), 7.93 (dd, J=8.2, 1.5 Hz, 2H), 7.27 (dd, J=8.0, 8.2 Hz, 2H), 6.88 (t, J=8.0 Hz, 1H), 6.10 (t, J=2.2 Hz, 1H), 6.04 (dd, J=8.0, 2.2 Hz, 2H).

### Intermediate 44-2

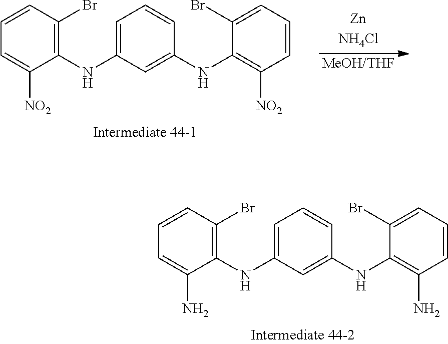 [SMILES: O=[N+]([O-])c1cccc(Br)c1Nc1cccc(Nc2c(Br)cccc2[N+](=O)[O-])c1 ; "MeOH/THF" ; Nc1cccc(Br)c1Nc1cccc(Nc2c(N)cccc2Br)c1]

11.0 g (21.7 mmol) of Intermediate 44-1 were dissolved in 150 ml of methanol and 15 ml of tetrahydrofuran, and heated to 40° C. 46.3 g (866 mmol) of ammonium chloride were added, and the reaction mixture was cooled to room temperature. 28.3 g (433 mmol) of zinc dust were added in 4 portions, keeping the internal temperature below 40° C. The reaction mixture was then filtered over Celite, the cake washed with tetrahydrofuran, and the solvent of the filtrate was removed on the rotavap. Water and dichloromethane were then added to the resulting crude product, and the organic extracts were washed with water, brine, dried over anhydrous magnesium sulfate, and filtered. The solvent from the filtrate was removed on the rotavap, and the resulting solid was triturated in ethanol and filtered to give 6.91 g (64% yield) of Intermediate 44-2.

¹H NMR (300 MHz, DMSO-d₆) δ 6.94 (s, 2H), 6.91-6.69 (m, 7H), 5.89 (t, J=2.1 Hz, 1H), 5.75 (dd, J=8.0, 2.1 Hz, 2H), 4.98 (s, 4H).

### Intermediate 44-3

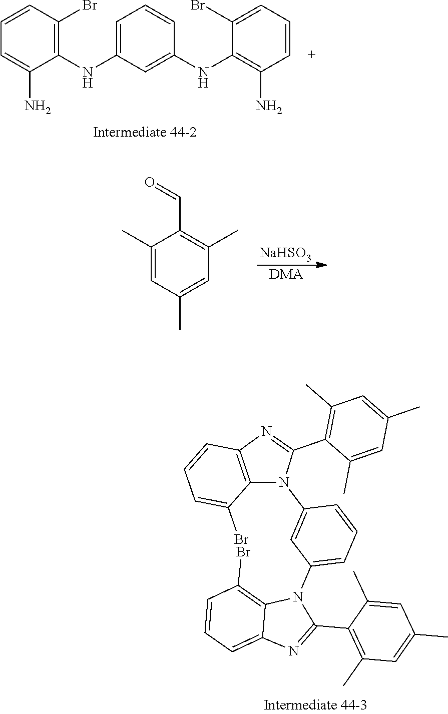

3.0 g (6.69 mmol) of Intermediate 44-2 were dissolved in 33 ml of N,N-dimethylacetamide, 16.7 g (161 mmol) of sodium bisulfite were added, and the reaction was heated to 110° C. A solution of 4.0 g (26.8 mmol) of 2,4,6-trimethylbenzaldehyde in 33 ml of N,N-dimethylacetamide was added slowly to the reaction mixture, and when the addition was complete the reaction was heated to 130° C. for 24 hours. After cooling to room temperature, the reaction mixture was poured into water and extracted with dichloromethane. The organic extracts were washed with water, brine, dried over anhydrous magnesium sulfate, and filtered. The solvent from the filtrate was removed on the rotavap, and the crude product was purified by column chromatography (220 g silica, dichloromethane/tetrahydrofuran=98/2) to give 4.1 g (87% yield) of Intermediate 44-3 as an off-white foam.

¹H NMR (300 MHz, DMSO-d₆) δ 7.78 (dd, J=8.0, 1.0 Hz, 2H), 7.53-7.37 (m, 6H), 7.22 (t, J=7.9 Hz, 2H), 6.83 (s, 4H), 2.21 (s, 6H), 1.99 (s, 6H), 1.98 (s, 6H).

### Compound 44

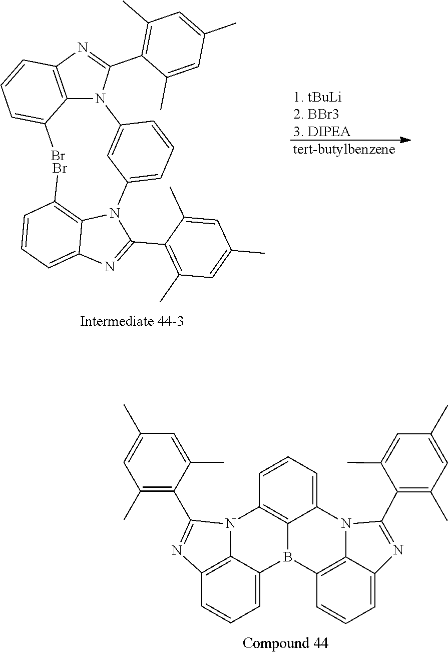 [SMILES: Cc1cc(C)c(-c2nc3cccc(Br)c3n2-c2cccc(-n3c(-c4c(C)cc(C)cc4C)nc4cccc(Br)c43)c2)c(C)c1 ; "1. tBuLi2. BBr33. DIPEA" ; "tert-butylbenzene" ; Cc1cc(C)c(-c2nc3cccc4c3n2-c2cccc3c2B4c2cccc4nc(-c5c(C)cc(C)cc5C)n-3c24)c(C)c1]

1.5 g (2.13 mmol) of Intermediate 44-3 were dissolved in 50 ml of anhydrous tert-butylbenzene under inert atmosphere. 3.5 ml of tert-butyllithium (6.6 mmol, 1.9M in pentane) were added dropwise to the yellow solution, keeping the internal temperature below 30° C. After 90 minutes, the brown precipitate was cooled to −78° C., and 0.81 ml (8.52 mmol) of BBr3 were added dropwise, and the reaction left to reach room temperature slowly. After reaching room temperature, the reaction was cooled to 0° C. and 1.5 ml (8.52 mmol) of N,N-diisopropylethylamine were added in one portion, and the reaction was heated to 100° C. for 1 hour, during which a yellow sticky precipitate formed. Reaction was cooled, and quenched with water, and extracted with ethyl acetate. The organic extracts were washed with water, brine, dried over anhydrous magnesium sulfate, and filtered. The solvent from the filtrate was removed on the rotavap, and the crude product was purified by column chromatography (120 g silica, heptane/ethylacetate/methanol=75/22.5/2.5) to give 35 mg (3% yield) of Compound 44 as a yellow shiny solid.

¹H NMR (300 MHz, DMSO-d₆) δ 8.41 (d, J=6.8 Hz, 2H), 7.84 (d, J=8.0 Hz, 2H), 7.55 (m, 2H), 7.12 (s, 4H), 6.99 (t, J=8.3 Hz, 1H), 6.78 (d, J=8.3 Hz, 2H), 2.38 (s, 6H), 1.99 (s, 12H).

### Compound 45

### Intermediate 45-1

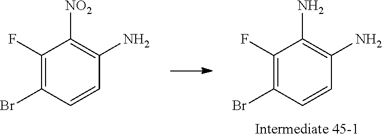 [SMILES: Nc1ccc(Br)c(F)c1[N+](=O)[O-] ; Nc1ccc(Br)c(F)c1N]

To 32.7 g (139 mmol) of 3-bromo-2-fluoro-6-nitroaniline in 250 ml THF, 59.5 g (1.11 mol) of ammonium chloride in 200 ml water were added under argon and stirring. 36.4 g (557 mmol) of zinc powder were added. The reaction mixture was stirred for 2 h at 65° C. The solids were filtered off and then ethyl acetate was added. The organic phase was separated and was washed with water, sodium hydrogen carbonate solution and brine. The organic phase was dried with magnesium sulfate. Column chromatography with heptane/ethyl acetate 80/20 and then 80/40 gave 21.3 g of the Intermediate 45-1 (74% yield).

¹H-NMR (400 MHz, DMSO-d6)⋅=6.57 (dd, 1H), 6.31 (m, 1H), 4.94 (s, 2H), 4.66 (s, 2H).

### Intermediate 45-2

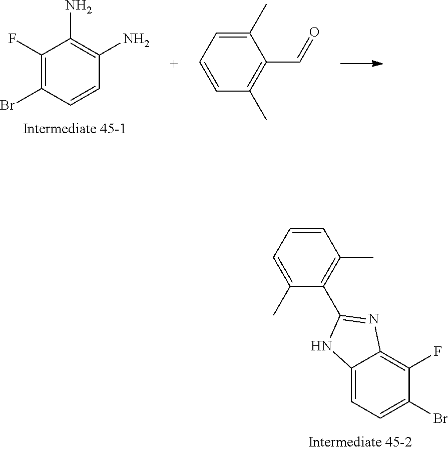

To a solution of 21.4 g (104 mmol) of Intermediate 45-1 in 75 ml of DMA, a solution of 10.9 g (104 mmol) sodium bisulfite in 75 ml of DMA was added at 125° C. within 2.5 h. Then a solution of 14.0 g (104 mmol) of 2,6-dimethylbenzaldehyde in 75 ml of DMA was added at 125° C. over a period of 10 min. The reaction mixture was stirred at 125° C. for 12 h under nitrogen. The solids were filtered off and the organic solvent was removed in vacuum. Ethyl acetate was added and the organic phase was washed with brine and was dried with magnesium sulfate to give 16.0 g of Intermediate 45-2 (41% yield).

¹H-NMR (400 MHz, DMSO-d6)⋅=13.1 (s, 1H), 7.40 (m, 3H), 7.21 (d, 2H), 7.11 (s, 6H).

### Intermediate 45-3

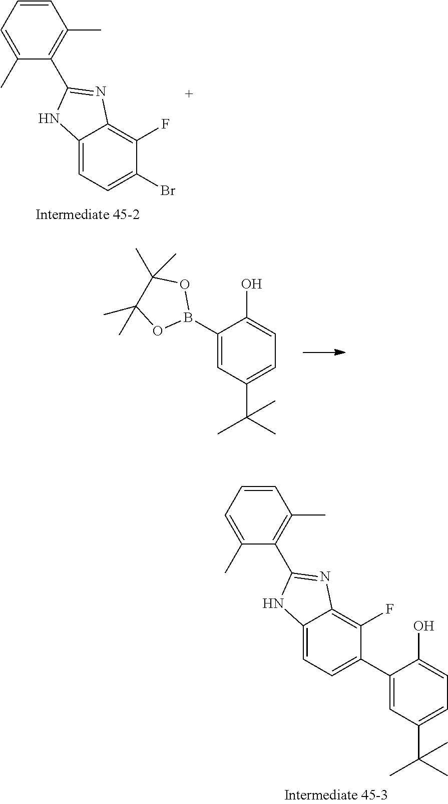 [SMILES: Cc1cccc(C)c1-c1nc2c(F)c(Br)ccc2[nH]1 ; CC(C)(C)c1ccc(O)c(B2OC(C)(C)C(C)(C)O2)c1 ; Cc1cccc(C)c1-c1nc2c(F)c(-c3cc(C(C)(C)C)ccc3O)ccc2[nH]1]

To 24.6 g (77.0 mmol) of Intermediate 45-2, 21.3 g (77.0 mmol) of 4-(tert-butyl)-2-(4,4,5,5-tetramethyl-1,3,2-dioxaborolan-2-yl)phenol (prepared according to the procedure given in J. Am. Chem. Soc. 2017, 139, 7864-7871) and 26.8 g (154 mmol) of potassium hydrogen phosphate in 120 ml of dioxane, 90 ml of water and 270 ml of toluene were degassed with argon. 1.08 g (1.54 mmol) of bis(triphenylphosphine)-palladium(II) dichloride were added and the reaction mixture was degassed with argon. The reaction mixture was stirred at 95° C. for 18 h. The reaction mixture was cooled to 25° C. and the organic phase was removed. The solvent was removed in vacuum and the product was purified by column chromatography on silica gel with heptane/ethyl acetate 80/20 and then 80/40 to give 15.3 g of Intermediate 45-3 (51% yield).

MS (ESI) m/z=389 (M+1)

### Intermediate 45-4

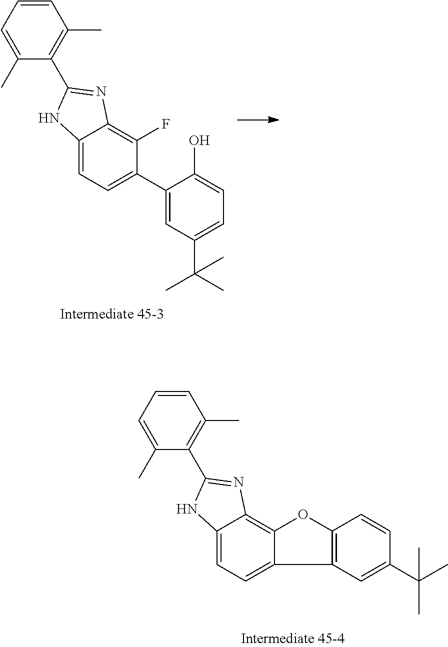 [SMILES: Cc1cccc(C)c1-c1nc2c(F)c(-c3cc(C(C)(C)C)ccc3O)ccc2[nH]1 ; Cc1cccc(C)c1-c1nc2c(ccc3c4cc(C(C)(C)C)ccc4oc32)[nH]1]

To 15.3 g (39.4 mmol) of Intermediate 45-3 in 130 ml of NMP, 16.7 g (79.0 mmol) of tripotassium phosphate were added under argon. The reaction mixture was stirred at 145° C. for 5 h under argon. The solids were filtered off and the solvent was removed in vacuum. Column chromatography on silica gel with heptane/ethyl acetate 80/20 gave the Intermediate 45-4.

¹H-NMR (400 MHz, DMSO-d6)⋅=13.1 (s, 1H), 8.16 (d, 1H), 8.00 (d, 1H), 7.67 (d, 1H), 7.52 (m, 2H), 7.36 (dd, 1H), 7.23 (d, 2H), 2.16 (s, 6H), 9.13 (s, 9H).

### Intermediate 45-5

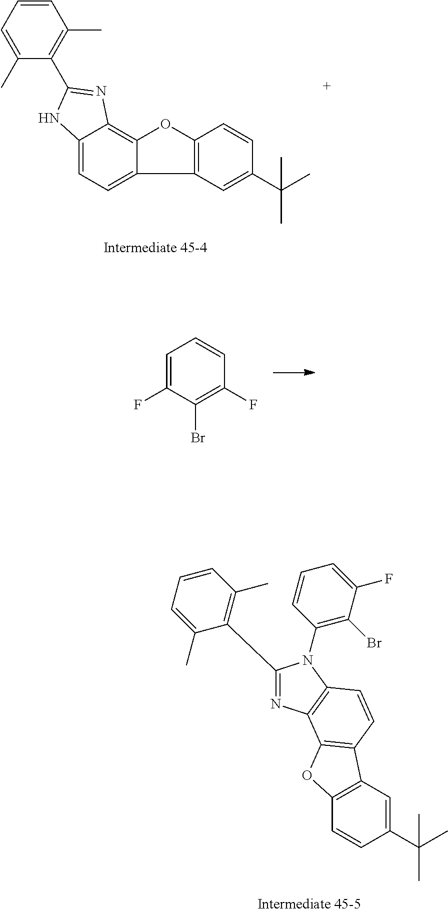 [SMILES: Cc1cccc(C)c1-c1nc2c(ccc3c4cc(C(C)(C)C)ccc4oc32)[nH]1 ; Fc1cccc(F)c1Br ; Cc1cccc(C)c1-c1nc2c3oc4ccc(C(C)(C)C)cc4c3ccc2n1-c1cccc(F)c1Br]

To 9.55 g (25.9 mmol) of Intermediate 45-4, 13.8 g (64.8 mmol) of tripotassium phosphate and 10.0 g (51.8 mmol) of 2-bromo-1,3-difluorobenzene in 100 ml of water free DMF were stirred under argon at 155° C. for 18 h. The solids were filtered of and the solvent was removed in vacuum. Column chromatography on silica gel with heptane/ethyl acetate 90/10 gave 8.00 g of the Intermediate 45-5 (51% yield).

¹H-NMR (400 MHz, DMSO-d6)⋅=8.21 (m, 1H), 8.12 (m, 1H), 7.73 (d, 1H), 7.52 (m, 3H), 7.22 (m, 4H), 7.05 (d, 1H), 2.22 (s, 3H), 2.08 (s, 3H), 1.43 (s, 9H).

MS (ESI) m/z=541 (M+1)

### Intermediate 45-6

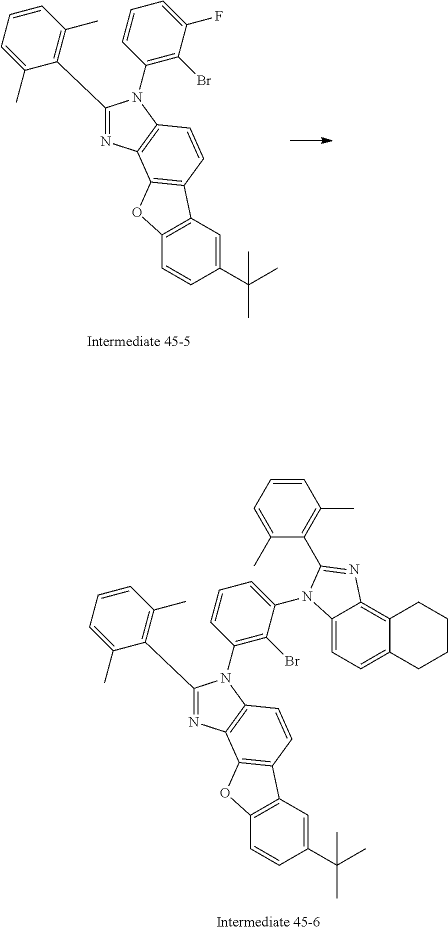 [SMILES: Cc1cccc(C)c1-c1nc2c3oc4ccc(C(C)(C)C)cc4c3ccc2n1-c1cccc(F)c1Br ; Cc1cccc(C)c1-c1nc2c3c(ccc2n1-c1cccc(-n2c(-c4c(C)cccc4C)nc4c5oc6ccc(C(C)(C)C)cc6c5ccc42)c1Br)CCCC3]

To 536 mg (1.94 mmol) of 2-(2,6-dimethylphenyl)-6,7,8,9-tetrahydro-1H-naphtho[1,2-d]imidazole and 1.03 g (4.85 mmol) of tripotassium phosphate in 8 ml of water free DMF, 1.05 g (1.94 mmol) of Intermediate 45-5 were added under argon. The reaction mixture was stirred for 18 h at 155° C. under argon. The solids were filtered off and the solvent was removed in vacuum. Column chromatography on silica gel with heptane/ethyl acetate 95/5 gave 1.16 g of the Intermediate 45-6 (71% yield).

MS (ESI) m/z=797 (M+1)

### Compound 45

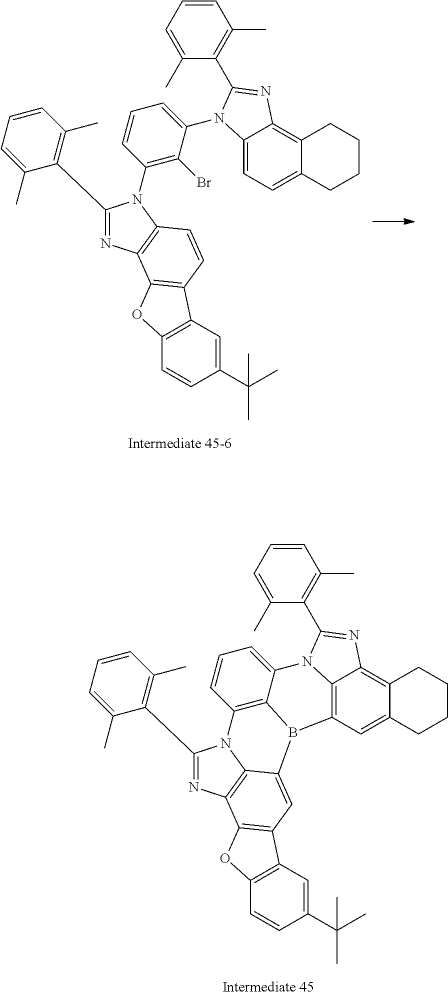 [SMILES: Cc1cccc(C)c1-c1nc2c3c(ccc2n1-c1cccc(-n2c(-c4c(C)cccc4C)nc4c5oc6ccc(C(C)(C)C)cc6c5ccc42)c1Br)CCCC3 ; Cc1cccc(C)c1-c1nc2c3c(cc4c2n1-c1cccc2c1B4c1cc4c5cc(C(C)(C)C)ccc5oc4c4nc(-c5c(C)cccc5C)n-2c14)CCCC3]

To 1.05 g (1.32 mmol) of Intermediate 45-6 in 20 ml of water free tert-butylbenzene, 1.47 ml (2.50 mmol) of tert-butyllithium in heptane was added at 0° C. under argon. The reaction mixture was stirred for 90 min at 25° C. under argon. The reaction mixture was cooled to −40° C. and 0.989 g (3.95 mmol) of tribromoborane were added under argon. The reaction mixture was stirred for 60 min at −40° C. and was then warmed up to 25° C. After 30 min the reaction mixture was cooled to 0° C. and 0.510 g (3.95 mmol) of di-isopropyl-ethylamine were added. After 15 min the reaction mixture was stirred at 150° C. for 18 h. The reaction mixture was cooled to 25° C. 20 ml of ammonium hydrogen chloride solution and ethyl acetate were added and the organic phase was separated. The organic phase was dried with sodium sulfate and the solvent was removed in vacuum. Column chromatography on silica gel with heptane/ethyl acetate 90/10 gave 30 mg of the Compound 45 (3% yield).

MS (ESI) m/z=727 (M+1)

### Compound 47

### Intermediate 47-1

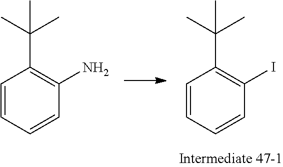

28.7 g (151 mmol) of p-toluenesulfonic acid monohydrate were dissolved in 75 ml of tert-butanol. 5.0 g (33.5 mmol) of 2-(tert-butyl)aniline were added, and the final white suspension was cooled to −3° C. A solution of 6.93 g (101 mmol) of sodium nitrite and 20.86 g (126 mmol) of potassium iodide in 30 ml of water was added within 30 minutes. It was warmed up to room temperature and stirred for 17 hours. 27.4 g (174 mmol) of sodium sulfurothioate were dissolved in 100 ml of water and added to the reaction mixture. 11.26 g (134 mmol) of sodium bicarbonate were dissolved in 100 ml of water and added, then the mixture was stirred for 30 minutes. The reaction mixture was diluted with 200 ml of cyclohexane, the phases were separated and the water phase was extracted with cyclohexane. The combined organic phases were washed with water and brine, dried over magnesium sulfate, filtered and concentrated under vacuum. The crude product was purified by column chromatography (heptane/ethyl acetate) to give 5.36 g (45% yield) of Intermediate 47-1.

¹H NMR (400 MHz, Methylene Chloride-d2) δ 8.00 (dd, J=7.8, 1.5 Hz, 1H), 7.46 (dd, J=8.0, 1.7 Hz, 1H), 7.36-7.23 (m, 1H), 6.89-6.78 (m, 1H), 1.54 (s, 9H).

### Intermediate 47-2

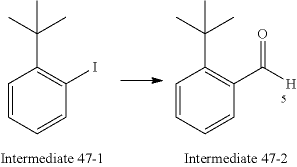

11.8 g (45.4 mmol) of Intermediate 47-1 were dissolved in 60 ml of tetrahydrofuran. The orange solution was bubbled with argon and cooled to −78° C. 18.48 ml (49.9 mmol) of n-butyllithium (2.7M in pentane) were added slowly and the reaction mixture was stirred for 30 minutes. 3.86 ml (49.9 mmol) of N,N-dimethylformamide were added at −78° C. and stirred for 30 minutes at this temperature, then it was heated up to room temperature and stirred for 18 hours. 100 ml of water and 100 ml of heptane were added, the phases were separated and the water phase was extracted with heptane. The combined organic phases were washed with water and brine, dried over magnesium sulfate, filtered and concentrated under vacuum to give 9.24 g (86% yield) of Intermediate 47-2.

¹H NMR (300 MHz, Methylene Chloride-d2) δ 10.83 (d, J=0.9 Hz, 1H), 7.89 (dt, J=7.6, 1.1 Hz, 1H), 7.58-7.44 (m, 2H), 7.40-7.23 (m, 1H), 1.52 (s, 9H).

### Intermediate 47-3

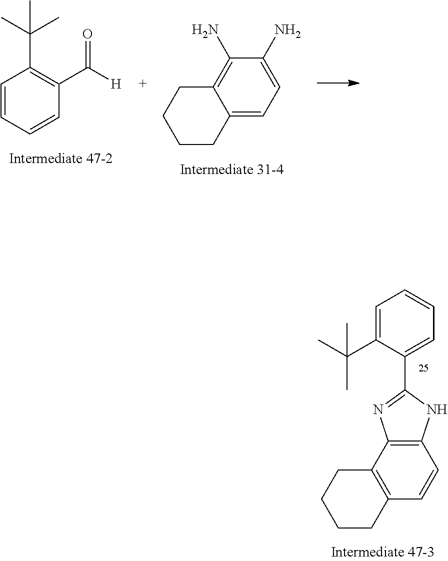 [SMILES: CC(C)(C)c1ccccc1C=O ; Nc1ccc2c(c1N)CCCC2 ; CC(C)(C)c1ccccc1-c1nc2c3c(ccc2[nH]1)CCCC3]

To 0.99 g (6.10 mmol) of Intermediate 31-4 in 10 ml of N,N-dimethylacetamide, 0.635 g (6.10 mmol) of sodium bisulfite were added and heated to 100° C. 0.99 g (6.10 mmol) of Intermediate 47-2 in 6 ml of N,N-dimethylacetamide were added dropwise at 100° C., then it was stirred at this temperature for 1 hour. The reaction mixture was cooled to room temperature and poured on 100 ml of water, filtered and the residue was washed with water. The isolated solid was suspended in 100 ml of heptane, stirred for 1 hour, filtered, washed with 100 ml of heptane and dried to give 0.825 g (44.4% yield) of Intermediate 47-3.

¹H NMR (300 MHz, Methylene Chloride-od) δ 9.29 (s, 1H), 7.60 (dt, J=8.1, 1.0 Hz, 1H), 7.44 (ddd, J=8.1, 5.0, 3.8 Hz, 1H), 7.26 (tt, J=4.3, 2.3 Hz, 3H), 6.99 (d, J=8.2 Hz, 1H), 3.09-2.78 (m, 4H), 2.01-1.78 (m, 4H), 1.26 (s, 9H).

### Intermediate 47-4

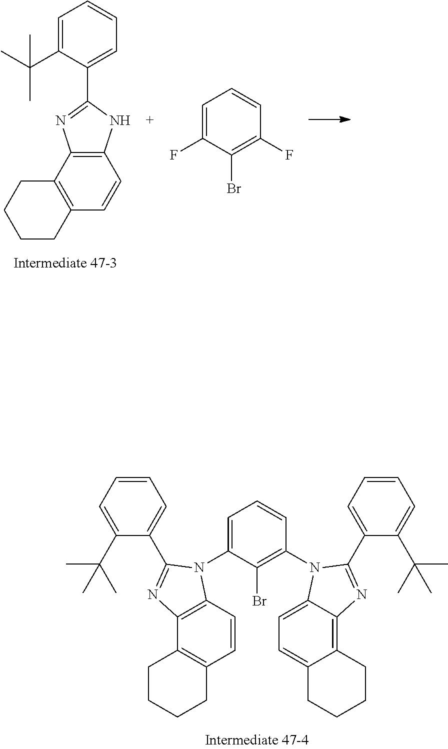 [SMILES: CC(C)(C)c1ccccc1-c1nc2c3c(ccc2[nH]1)CCCC3 ; Fc1cccc(F)c1Br ; CC(C)(C)c1ccccc1-c1nc2c3c(ccc2n1-c1cccc(-n2c(-c4ccccc4C(C)(C)C)nc4c5c(ccc42)CCCC5)c1Br)CCCC3]

4.73 g (15.54 mmol) of Intermediate 47-3 and 1.10 g (5.7 mmol) of 2-bromo-1,3-difluorobenzene were dissolved in 40 ml of N,N-dimethylformamide. 6.05 g (28.5 mmol) of potassium phosphate tribasic were added. The reaction mixture was heated at 150° C. (outside temperature) for 22 hours, then cooled to room temperature, filtered and the residue washed with N,N-dimethylformamide. The filtrate was concentrated under vacuum and the isolated solid was dissolved in 100 ml of ethyl acetate, then 60 ml of water were added. The phases were separated, the water phase was extracted with ethyl acetate, the combined organic phases were washed with water and brine, dried over magnesium sulfate, filtered and the solvent was evaporated under reduced pressure. The isolated solid was purified by column chromatography (heptane/ethyl acetate) to give 2 separated rotamers of Intermediate 47-4. (Rotamer 1: 1.83 g (42% yield), Rotamer 2: 1.27 g (29% yield)).

Rotamer 1:

¹H NMR (400 MHz, Methylene Chloride-d2) δ 7.61 (dd, J=8.2, 1.2 Hz, 2H), 7.44-7.15 (m, 9H), 7.07 (d, J=8.3 Hz, 2H), 6.61 (d, J=8.2 Hz, 2H), 3.32-3.10 (m, 4H), 2.92 (q, J=4.2 Hz, 4H), 1.93 (tdt, J=10.3, 7.8, 5.4 Hz, 8H), 1.41 (s, 18H)

Rotamer 2:

¹H NMR (400 MHz, Methylene Chloride-d2) δ 7.59 (dd, J=8.2, 1.2 Hz, 2H), 7.34 (ddd, J=8.5, 7.1, 1.7 Hz, 2H), 7.25-7.05 (m, 9H), 6.86 (d, J=8.2 Hz, 2H), 3.30-3.11 (m, 4H), 2.93 (q, J=4.2 Hz, 4H), 2.00-1.86 (m, 8H), 1.41 (s, 18H).

### Compound 47

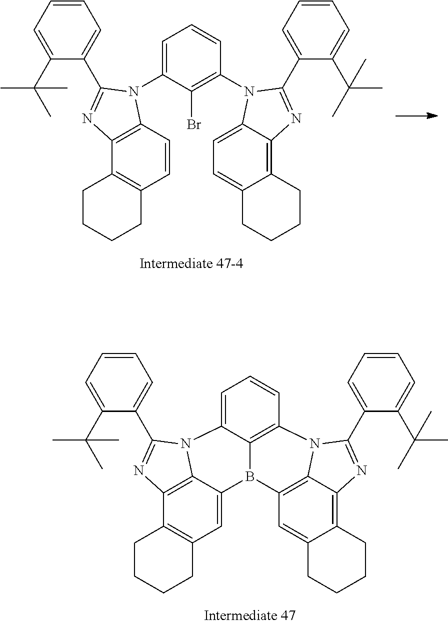

1.20 g (1.575 mmol) of Intermediate 47-4 were dissolved in 20 ml of water-free tert-butylbenzene. 0.91 ml (1.729 mmol) of tert-butyllithium (1.9M in pentane) were slowly added at 40° C. and stirred for 30 minutes. The yellowish solution was cooled down to −35° C. and 0.60 ml (6.35 mmol) of tribromoborane were added. The reaction mixture was warmed up to room temperature, stirred for 2 hours and then cooled down to 0° C. 1.10 ml (6.30 mmol) of N,N-diisopropylethylamine were added and the reaction mixture was heated at 140° C. for 30 minutes. The yellow suspension was cooled down to room temperature and treated with 20 ml of 10% aqueous sodium acetate solution and extracted with 80 ml of ethyl acetate. The organic layer was separated, washed with water, dried over magnesium sulfate, filtered and concentrated under vacuum. The isolated product was purified by column chromatography (heptane/methanol). The product was further suspended in a mixture of dichloromethane and ethyl acetate, filtered and dried to give 0.473 g (43.5% yield) of Compound 47 as a mixture of rotamers.

¹H NMR (300 MHz, Methylene Chloride-d2) δ 8.59 (s, 2H), 7.75 (dt, J=8.2, 1.3 Hz, 2H), 7.64-7.52 (m, 2H), 7.38-7.29 (m, 2H), 7.24 (ddd, J=9.3, 7.6, 1.7 Hz, 2H), 7.04 (ddd, J=9.0, 7.9, 1.1 Hz, 1H), 6.80 (dd, J=8.4, 4.9 Hz, 2H), 3.38 (d, J=5.0 Hz, 4H), 3.22 (s, 4H), 2.03 (s, 8H), 1.25 (s, 18H).

### Compound 48

### Intermediate 48-1

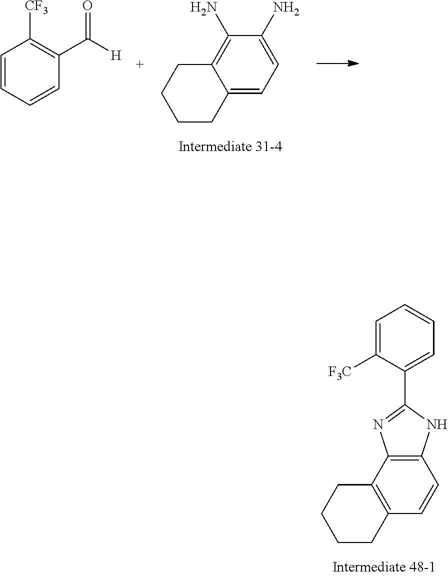 [SMILES: O=Cc1ccccc1C(F)(F)F ; Nc1ccc2c(c1N)CCCC2 ; FC(F)(F)c1ccccc1-c1nc2c3c(ccc2[nH]1)CCCC3]

To 1.126 g (6.94 mmol) of Intermediate 31-4 in 10 ml of N,N-dimethylacetamide, 0.726 g (8.95 mmol) of sodium bisulfite were added and heated to 100° C. 1.048 g (6.02 mmol) of 2-(trifluoromethyl)benzaldehyde in 6 ml of N,N-dimethylacetamide were added dropwise at 100° C., then it was stirred at this temperature for 22 hours. The reaction mixture was cooled to room temperature and poured on 100 ml of water, filtered and the residue was washed with 100 ml of water. The isolated solid was suspended in 100 ml of heptane, stirred for 1 hour, filtered, washed with 100 ml of heptane and dried to give 1.90 g (100% yield) of Intermediate 48-1.

¹H NMR (300 MHz, Methylene Chloride-d2) δ 7.96-7.89 (m, 1H), 7.83 (dd, J=7.7, 1.6 Hz, 1H), 7.74-7.57 (m, 2H), 7.39 (d, J=8.2 Hz, 1H), 7.03 (d, J=8.3 Hz, 1H), 2.99 (q, J=4.7, 3.9 Hz, 2H), 2.89 (q, J=5.3, 4.6 Hz, 2H), 1.97-1.79 (m, 4H).

### Intermediate 48-2

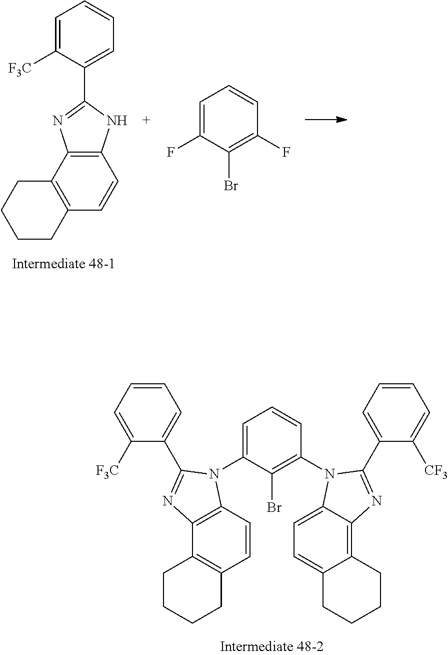 [SMILES: FC(F)(F)c1ccccc1-c1nc2c3c(ccc2[nH]1)CCCC3 ; Fc1cccc(F)c1Br ; FC(F)(F)c1ccccc1-c1nc2c3c(ccc2n1-c1cccc(-n2c(-c4ccccc4C(F)(F)F)nc4c5c(ccc42)CCCC5)c1Br)CCCC3]

2.028 g (6.41 mmol) of Intermediate 48-1 and 0.63 g (3.26 mmol) of 2-bromo-1,3-difluorobenzene were dissolved in 15 ml of N,N-dimethylformamide. 2.475 g (11.66 mmol) of potassium phosphate tribasic were added. The reaction mixture was heated at 150° C. (outside temperature) for 21 hours, then cooled to room temperature, filtered and the residue washed with N,N-dimethylformamide. The filtrate was concentrated under vacuum and the isolated solid was dissolved in 100 ml of ethyl acetate, then 60 ml of water were added. The phases were separated, the water phase was extracted with ethyl acetate, the combined organic phases were washed with water and brine, dried over magnesium sulfate, filtered and the solvent was evaporated under reduced pressure. The isolated solid was purified by column chromatography (heptane/ethyl acetate) to give 1.76 g (96% yield) of Intermediate 48-2 as a mixture of rotamers.

LC-MS: 783.1 [M−H]⁻

### Compound 48

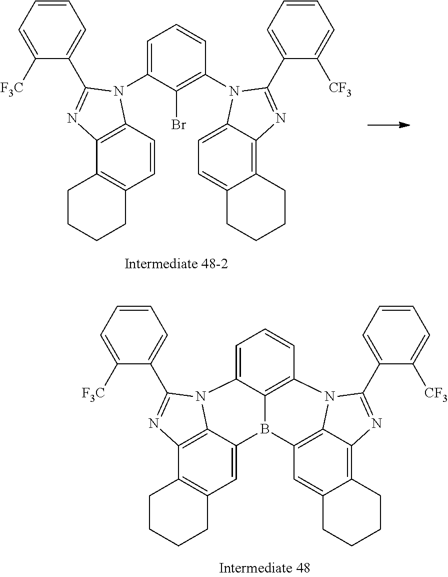 [SMILES: FC(F)(F)c1ccccc1-c1nc2c3c(ccc2n1-c1cccc(-n2c(-c4ccccc4C(F)(F)F)nc4c5c(ccc42)CCCC5)c1Br)CCCC3 ; FC(F)(F)c1ccccc1-c1nc2c3c(cc4c2n1-c1cccc2c1B4c1cc4c(c5nc(-c6ccccc6C(F)(F)F)n-2c15)CCCC4)CCCC3]

0.830 g (1.056 mmol) of Intermediate 48-2 were dissolved in 20 ml of water-free tert-butylbenzene. 0.610 ml (1.159 mmol) of tert-butyllithium (1.9M in pentane) were slowly added at 50° C. and stirred for 1 hour. The yellowish solution was cooled down to −35° C. and 0.40 ml (4.23 mmol) of tribromoborane were added. The reaction mixture was warmed up to room temperature, stirred for 2 hours and then cooled down to 0° C. 0.738 ml (4.23 mmol) of N,N-diisopropylethylamine were added and the reaction mixture was heated at 150° C. for 1 hour. The yellow suspension was cooled down to room temperature and treated with 20 ml of 10% aqueous sodium acetate solution and extracted with 80 ml of ethyl acetate. The organic layer was separated, washed with water, dried over magnesium sulfate, filtered and concentrated under vacuum. The isolated product was purified by column chromatography (heptane/ethyl acetate/methanol) to give 0.060 g (8% yield) of Compound 48 as a mixture of rotamers.

LC-MS: 713.2 [M−H]⁻

### Compound 49

### Intermediate 49-1

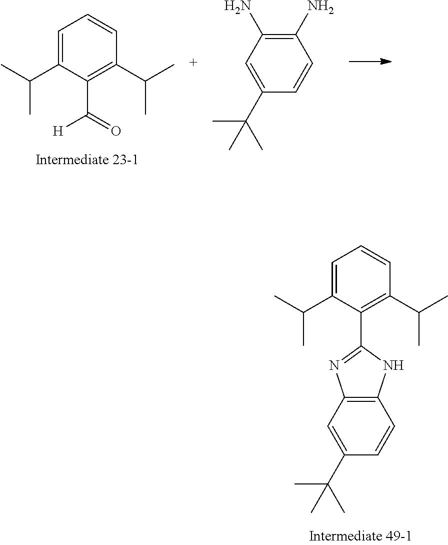 [SMILES: CC(C)c1cccc(C(C)C)c1C=O ; CC(C)(C)c1ccc(N)c(N)c1 ; CC(C)c1cccc(C(C)C)c1-c1nc2cc(C(C)(C)C)ccc2[nH]1]

Preparation was done in analogy to Intermediate 31-5 with 25.0 g (152 mmol) of 4-(tert-butyl)benzene-1,2-diamine, 16.6 g (160 mmol) of sodium bisulfite, and 30.4 g (160 mmol) of Intermediate 23-1, in a total of 190 ml of N,N-dimethylacetamide, to give 39.8 g (78% yield) of Intermediate 49-1.

¹H NMR (300 MHz, DMSO-d₆) δ 12.37 (s, 1H), 7.64-7.38 (br. s and t, 3H), 7.33-7.24 (m, 3H), 2.49-2.34 (m, 2H), 1.37 (s, 9H), 1.11 (s, 6H), 1.09 (s, 6H).

### Intermediate 49-2

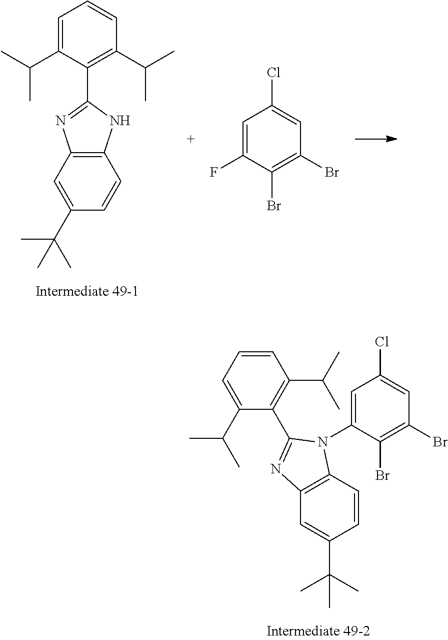 [SMILES: CC(C)c1cccc(C(C)C)c1-c1nc2cc(C(C)(C)C)ccc2[nH]1 ; Fc1cc(Cl)cc(Br)c1Br ; CC(C)c1cccc(C(C)C)c1-c1nc2cc(C(C)(C)C)ccc2n1-c1cc(Cl)cc(Br)c1Br]

Preparation was done in analogy to Intermediate 30-1 with 32 g (96 mmol) of Intermediate 49-1, 31 g (105 mmol) of 1,2-dibromo-5-chloro-3-fluorobenzene and 103 g (0.48 mol) of potassium phosphate in 300 ml of N,N-dimethylformamide, to give 7.7 g (13% yield) of Intermediate 49-2 as a white solid.

¹H NMR (300 MHz, CD₂Cl₂) δ 7.95 (d, 1H), 7.71 (d, 1H), 7.53-7.39 (m, 2H), 7.29 (dd, 1H), 7.17-7.10 (m, 2H), 7.07 (d, 1H), 2.75-2.62 (m, 1H), 2.62-2.47 (m, 1H), 1.49 (s, 9H), 1.38 (d, 3H), 1.29 (d, 3H), 1.10 (d, 3H), 0.95 (d, 3H).

### Intermediate 49-3

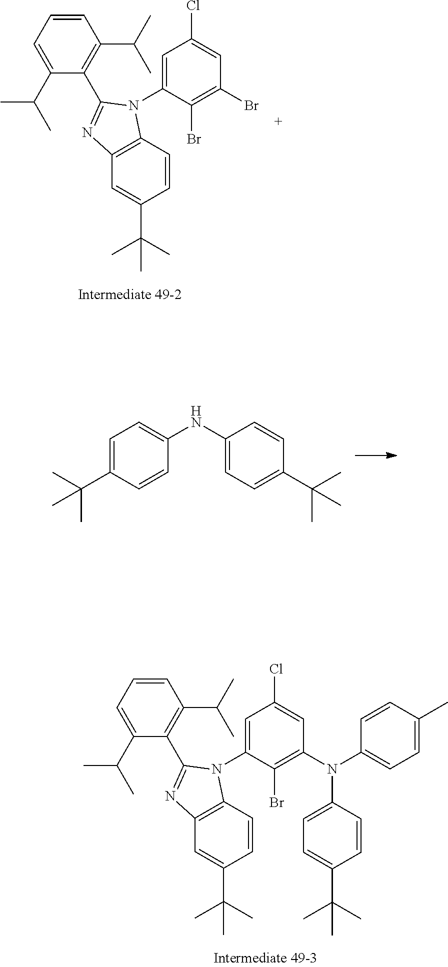

Preparation was done in analogy to Intermediate 30-2 with 6.70 g (11.1 mmol) of Intermediate 49-2, 4.0 g (14.2 mmol) of bis(4-(tert-butyl)phenyl)amine, 0.30 g (0.33 mmol) of tris(dibenzylideneacetone)dipalladium(0), 0.38 g (1.33 mmol) of tri-tert-butylphosphonium tetrafluoroborate, and 2.7 g (28 mmol) of sodium tert-butoxide in 120 ml of toluene, to give 7.1 g (69% yield) of Intermediate 49-3 as a white solid.

¹H NMR (300 MHz, CD₂Cl₂) δ 7.92 (br. s, 1H), 7.53-7.39 (m, 2H), 7.37-7.25 (m, 5H), 7.25-7.15 (m, 2H), 7.08-6.97 (m, 2H), 6.90-6.79 (m, 4H), 2.82-2.64 (m, 1H), 2.64-2.46 (m, 1H), 1.47 (s, 9H), 1.35 (s, 18H), 1.26 (d, 3H), 1.20 (d, 3H), 1.08 (d, 3H), 0.90 (d, 3H).

### Intermediate 49-4

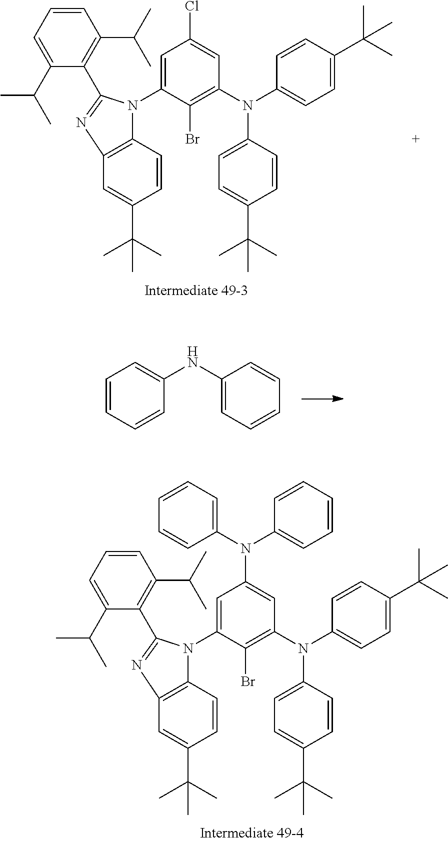

Preparation was done in analogy to Intermediate 30-3 with 15.6 g (19.4 mmol) of Intermediate 49-3, 4.3 g (25.4 mmol) of diphenylamine, 0.54 g (0.59 mmol) of tris(dibenzylideneacetone)dipalladium(0), 0.68 g (2.34 mmol) of tri-tert-butylphosphonium tetrafluoroborate, and 4.8 g (50 mmol) of sodium tert-butoxide in 300 ml of toluene, to give 5.6 g (31% yield) of Intermediate 49-4 as a white solid.

¹H NMR (300 MHz, CD₂Cl₂) δ 7.87 (br. s, 1H), 7.57-7.38 (m, 3H), 7.38-7.05 (m, 11H), 7.05-6.95 (m, 2H), 6.93-6.78 (m, 8H), 6.66 (d, 1H), 2.90-2.71 (m, 1H), 2.36-2.16 (m, 1H), 1.47 (s, 9H), 1.35 (s, 18H), 1.15 (d, 3H), 1.06 (d, 3H), 0.96 (d, 3H), 0.56 (d, 3H).

### Compound 49

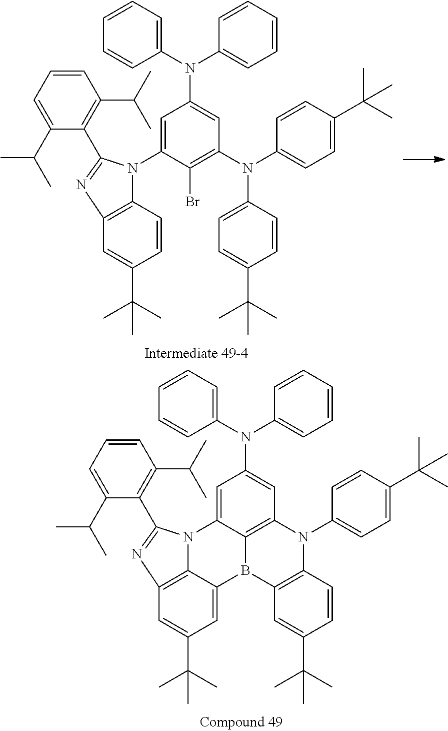

Preparation was done in analogy to Compound 30 with 5.6 g (4.79 mmol) of Intermediate 49-4, 5.1 ml of tert-butyllithium (1.9 M in pentane), 0.95 ml (9.57 mmol) of tribromoborane, in 70 ml of water-free tert-butylbenzene, and five times feeding of 1.7 ml of N,N-diisopropylethyl-amine periodically over the whole reaction time, to give 0.89 g (22% yield) of Compound 49 as a yellow solid.

¹H NMR (300 MHz, CD₂Cl₂) δ 9.11 (d, 1H), 8.95 (br. s, 1H), 8.14 (br. s, 1H), 7.58 (dd, 1H), 7.54-7.46 (m, 2H), 7.27-6.96 (m, 11H), 6.87-6.72 (m, 5H), 6.46 (br. s, 1H), 5.90 (br. s, 1H), 2.68-2.38 (m, 2H), 1.66 (s, 9H), 1.53 (s, 9H), 1.36 (s, 9H), 1.12 (d, 6H), 1.01 (d, 6H).

### Compound 50

### Intermediate 50-1

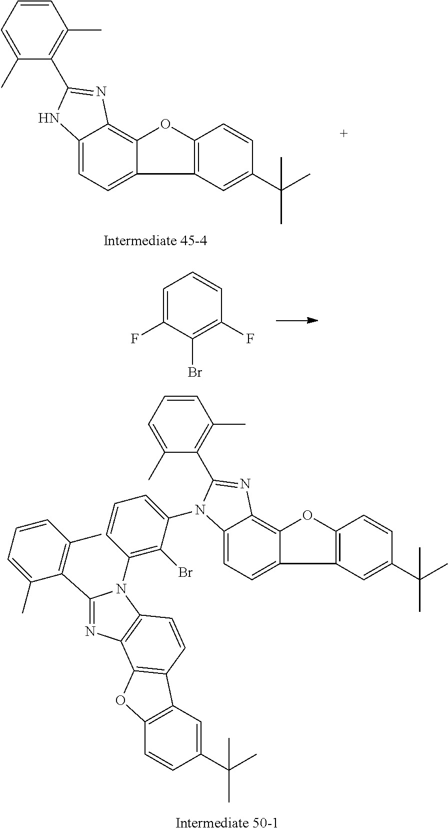

To 4.96 g (14.5 mmol) of Intermediate 45-4, 5.72 g (26.9 mmol) of tripotassium phosphate in 30 ml of water free DMF, 1.30 g (6.74 mmol) of 2-bromo-1,3-difluorobenzene were added under argon. The reaction mixture was stirred under argon for 18 h. The solids were filtered off and the solvent was removed in vacuum. Column chromatography on silica gel with heptane/ethyl acetate 90/10 and then 80/20 gave 1.26 g of the Intermediate 50-1 (34% yield).

¹H-NMR (400 MHz, DMSO-d6)⋅=8.22 (d, 2H), 8.17 (d, 2H), 7.72 (d, 2H), 7.57 (dd, 2H), 7.49 (m, 3H), 7.42 (d, 2H), 7.28 (t, 2H), 7.10 (d, 2H), 7.01 (d, 2H), 2.16 (s, 6H), 1.93 (s, 6H), 1.44 (s, 18H).

### Compound 50

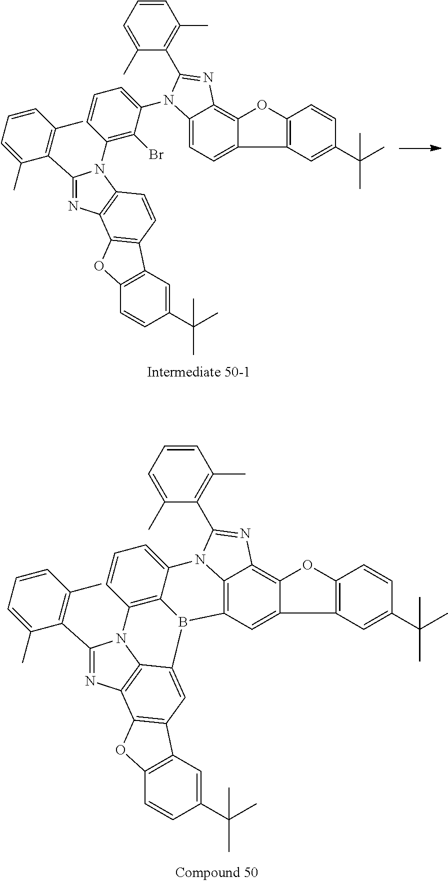 [SMILES: Cc1cccc(C)c1-c1nc2c3oc4ccc(C(C)(C)C)cc4c3ccc2n1-c1cccc(-n2c(-c3c(C)cccc3C)nc3c4oc5ccc(C(C)(C)C)cc5c4ccc32)c1Br ; Cc1cccc(C)c1-c1nc2c3oc4ccc(C(C)(C)C)cc4c3cc3c2n1-c1cccc2c1B3c1cc3c4cc(C(C)(C)C)ccc4oc3c3nc(-c4c(C)cccc4C)n-2c13]

To 0.970 g (1.09 mmol) of Intermediate 50-1 in 20 ml of water free tert-butylbenzene, 1.22 ml (2.07 mmol) of tert-butyllithium in heptane were added at 0° C. under argon. The reaction mixture was stirred for 110 min at 25° C. under argon. The reaction mixture was cooled to −40° C. and 0.546 g (2.18 mmol) of tribromoborane were added under argon. The reaction mixture was stirred for 40 min at −40° C. and was then warmed up to 25° C. After 30 min the reaction mixture was cooled to 0° C. and 0.282 g (2.18 mmol) of di-isopropyl-ethylamine were added. After 15 min the reaction mixture was stirred at 150° C. for 18 h. The reaction mixture was cooled to 25° C. 20 ml of ammonium hydrogen chloride solution and ethyl acetate were added and the organic phase was separated. The organic phase was dried with sodium sulfate and the solvent was removed in vacuum. Column chromatography on silica gel with heptane/ethyl acetate 90/10 gave the Compound 50 in traces.

### Comparative Example 1: Comparative Compound 2

### Intermediate Comparative Compound 2-1

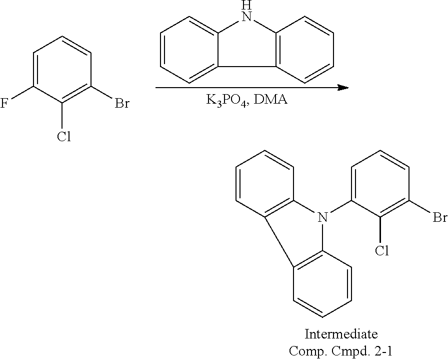 [SMILES: Fc1cccc(Br)c1Cl ; c1ccc2c(c1)[nH]c1ccccc12 ; Clc1c(Br)cccc1-n1c2ccccc2c2ccccc21]

16.7 g (100 mmol) of carbazole, 41.9 g (200 mmol) of 1-bromo-2-chloro-3-fluorobenzene, and 84.9 g (400 mmol) of potassium phosphate were suspended in 500 ml of N,N-dimethylacetamide, followed by heating at 138° C. overnight. The suspension was filtered, the solid washed with toluene, and the collected eluents concentrated under vacuum. The resulting oil was further purified by chromatography (silica gel, heptane/toluene 9:1), then taken up in dichloromethane and diluted with heptane. The solution was concentrated until a suspension formed. The suspension was filtered, giving Intermediate Comparative Compound 2-1 as a white solid (yield: 24.7 g (69%)).

¹H-NMR (400 MHz, CDCl₃): δ=8.20-8.15 (m, 2H), 7.87 (dd, 1H), 7.52 (dd, 1H), 7.47-7.30 (m, 5H), 7.12-7.07 (m, 2H).

### Intermediate Comparative Compound 2-2

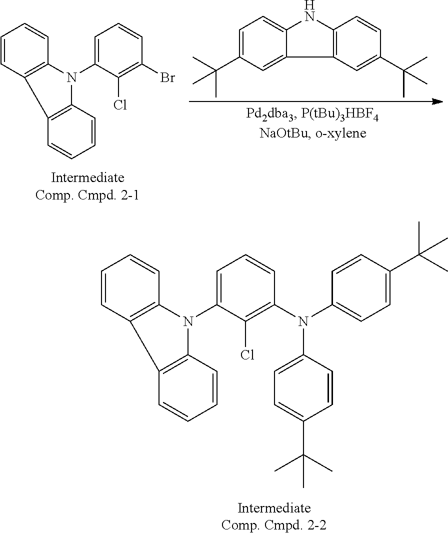

24.7 g (69.3 mmol) of Intermediate Comparative Compound 2-1, 19.7 g (69.9 mmol) of bis(4-(tert-butyl)phenyl)amine, 1.27 g (1.39 mmol) of tris(dibenzylideneacetone)dipalladium(0), 1.61 g (5.54 mmol) of tri-tert-butylphosphonium tetrafluoroborate, and 9.32 g (97.0 mmol) of sodium tert-butoxide were suspended in 230 ml of o-xylene. The suspension was three times evacuated and backfilled with argon and heated at 113° C. during 15 hours. The reaction mixture was filtered and 75 g of silica gel were added. The suspension was concentrated under vacuum, the solid further purified by chromatography (silica gel, heptane/toluene 9:1), and the product fractions concentrated under vacuum. The solid was dissolved in dichloromethane and diluted with ethanol. The solution was concentrated under vacuum until a suspension formed. The suspension was filtered and the solid washed with a small amount of ethanol. The solid was dissolved in dichloromethane and diluted with acetonitrile. The solution was concentrated under vacuum until a suspension formed, giving Intermediate Comparative Compound 2-2 as a white solid (yield: 30.2 g (78%)).

¹H-NMR (400 MHz, CDCl₃): δ=8.16 (d, 2H), 7.50-7.37 (m, 5H), 7.35-7.29 (m, 6H), 7.13 (d, 2H), 7.05-6.98 (m, 4H), 1.34 (s, 18H).

### Intermediate Comparative Compound 2-3

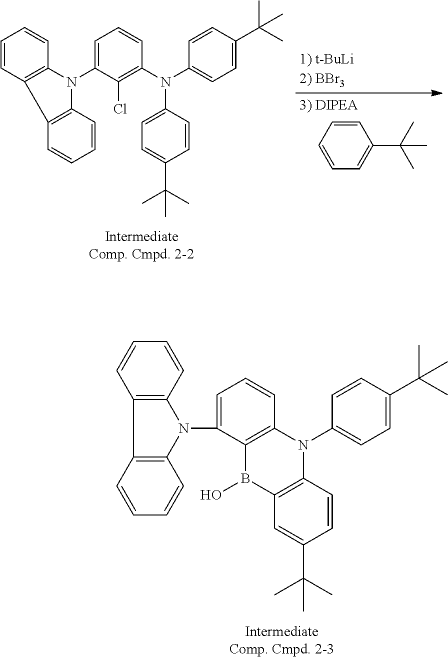

10.1 g (18.1 mmol) of Intermediate Comparative Compound 2-2 were dissolved in 144 ml of water-free tert-butyl benzene. 19.0 ml of tert-butyl lithium (1.9 M in pentane) were slowly added at −6° C. The yellow solution was heated up to 70° C. and pentane distilled off. The light brown solution was cooled down to −70° C. and 3.4 ml (36 mmol) of tribromoborane were slowly added. The reaction mixture was stirred at room temperature during 15 minutes and cooled down to 0° C. 6.3 ml (36 mmol) of N,N-diisopropylethylamine were added and the reaction mixture heated up to 113° C. during 16 hours. The brown suspension was poured into a mixture of 10% aqueous sodium acetate solution and toluene, and the organic layer separated. The aqueous layer was extracted twice with toluene. The combined organic layers were washed three times with water and once with brine, dried over sodium sulfate, and concentrated under vacuum. The brown oil was purified by chromatography (silica gel, heptane/toluene 6:1 to 2:1), giving Intermediate Comparative Compound 2-3 as a yellow solid (yield: 3.81 g (38%)).

¹H-NMR (400 MHz, DMSO-d₆): δ=8.26-8.18 (m, 2H), 8.03 (d, 1H), 7.84-7.76 (m, 2H), 7.66 (dd, 1H), 7.46 (dd, 1H), 7.42-7.36 (m, 2H), 7.36-7.29 (m, 2H), 7.29-7.12 (m, 3H), 7.11-7.02 (m, 3H), 6.79 (d, 1H), 6.43 (d, 1H), 1.46 (s, 9H), 1.19 (s, 9H).

### Comparative Compound 2

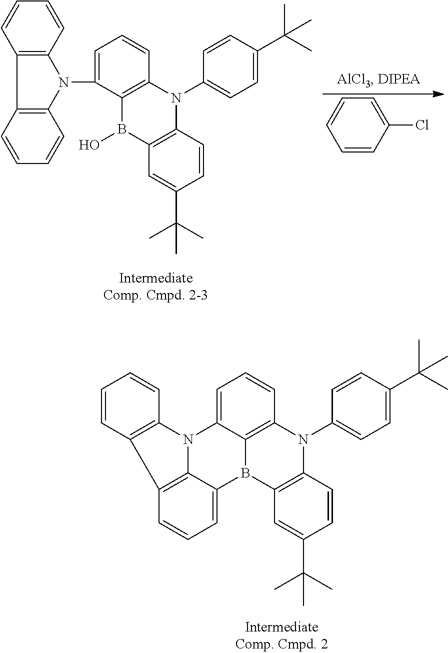 [SMILES: CC(C)(C)c1ccc(N2c3ccc(C(C)(C)C)cc3B(O)c3c2cccc3-n2c3ccccc3c3ccccc32)cc1 ; Clc1ccccc1 ; CC(C)(C)c1ccc(N2c3ccc(C(C)(C)C)cc3B3c4c2cccc4-n2c4ccccc4c4cccc3c42)cc1]

3.06 g (5.58 mmol) of Intermediate Comparative Compound 2-3 were dissolved in 28 ml of chlorobenzene. 7.44 g (55.8 mmol) of aluminium chloride and 4.9 ml (27.9 mmol) of N,N-diisopropylethylamine were slowly added, followed by heating at 120° C. during 4 hours. The reaction mixture was cooled down to room temperature and poured into an ice-water mixture, followed by extraction with toluene (three times). The combined organic layers were washed with brine, dried over sodium sulfate, and concentrated under vacuum. The yellow-brown solid was purified by MPLC with the CombiFlash Companion (silica gel, heptane/0-20% gradient of toluene). The yellow solid was dissolved in dichloromethane and diluted with heptane. The solution was concentrated under vacuum until a suspension formed. The suspension was filtered and the solid dissolved in dichloromethane and diluted with acetonitrile. The solution was concentrated under vacuum until a suspension formed. The precipitation from dichloromethane was repeated twice using heptane and 2-propanol as co-solvent, giving Comparative Compound 2 as a yellow solid (yield: 2.26 g (76%)).

¹H-NMR (400 MHz, DMSO-d₆): δ=8.94 (d, 1H), 8.88 (d, 1H), 8.68-8.55 (m, 2H), 8.45 (d, 1H), 8.20 (d, 1H), 7.87-7.75 (m, 4H), 7.75-7.64 (m, 2H), 7.51 (t, 1H), 7.45-7.37 (m, 2H), 6.70 (d, 1H), 6.47 (d, 1H), 1.47 (s, 9H), 1.46 (s, 9H).

### II Evaluation of Compounds

Next, the properties of the compounds used in the examples were measured. Measurement and calculation methods are shown below.

1.1 Device Application Data (Invented Compound as Emitter Dopant)

Preparation and Evaluation of Organic EL Devices

The organic EL devices were prepared and evaluated as follows:

### Comparative Application Example 1

A glass substrate (size: 25 mm×25 mm×0.7 mm, manufactured by Geomatec Co., Ltd.) bearing 130-nm-thick patterned ITO transparent electrodes (anode) was cleaned by N₂ plasma for 100 seconds. Afterwards, the substrate was mounted on the holder of a vacuum evaporation apparatus.

Initially, a host compound HT1 (the 1^{st} compound) and a dopant compound HI (the 2^{nd} compound) were co-vapor-deposited on the ITO patterned surface of the glass substrate, forming a 10-nm-thick hole injecting layer. The concentration of the compound HI in the hole injecting layer was given at 3 wt %.

Second, the compound HT1 was vapor-deposited on the hole injecting layer to form an 80-nm-thick 1^{st} hole transporting layer.

Third, the compound HT2 was vapor-deposited on the 1^{st} hole transporting layer to form a 10-nm-thick 2^{nd} hole transporting layer.

Further, the host compound BH1 (the 1^{st} compound) and the dopant compound (the 2^{nd} compound; Comparative Compound 1) were co-vapor-deposited on the 2^{nd} hole transporting layer to form a 25-nm-thick emitting layer. The concentration of the dopant compound in the emitting layer was given at 4 wt %.

Afterwards, the compound ET1 was vapor-deposited on the emitting layer to form a 5-nm-thick hole blocking layer.

Next, the compound ET2 was vapor-deposited on the hole blocking layer to form a 15-nm-thick electron transporting layer.

Lithium fluoride (LiF) was then vapor-deposited on the electron transporting layer to form a 1-nm-thick electron injecting layer.

Finally, metal aluminum (Al) was vapor-deposited on the electron injecting layer to form an 80-nm-thick metal Al cathode.

To characterize the OLED, electroluminescence spectra were recorded at various currents and voltages. In addition, the current-voltage characteristic was measured in combination with the luminance to determine luminous efficiency and external quantum efficiency (EQE). 90% lifetime (LT90), the time spent until the initial luminance at 50 mA/cm² was reduced to 90%, was recorded.

| | |
|---|---|
| 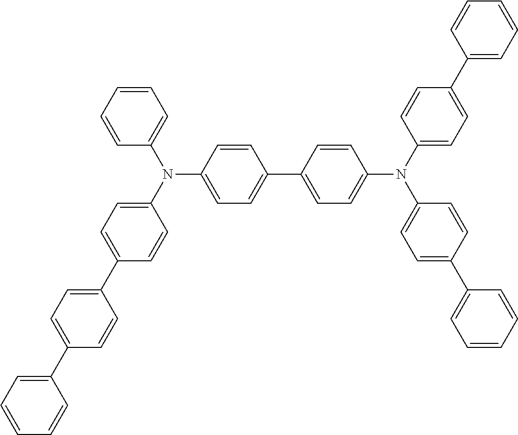 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1] | Compound HT1 |
| 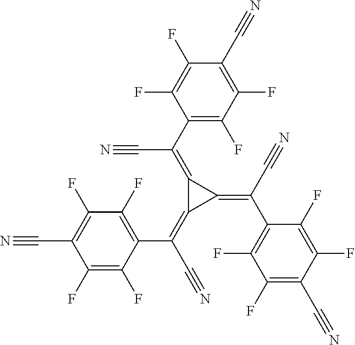 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] | Compound HI |
| 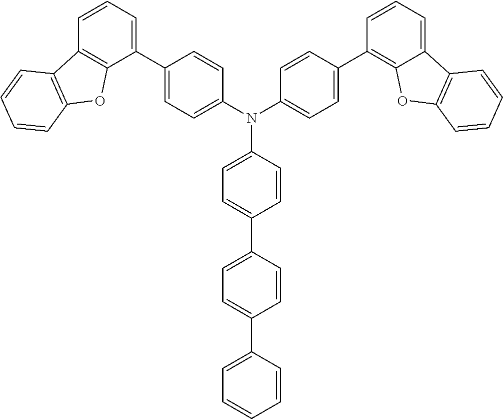 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1] | Compound HT2 |
| 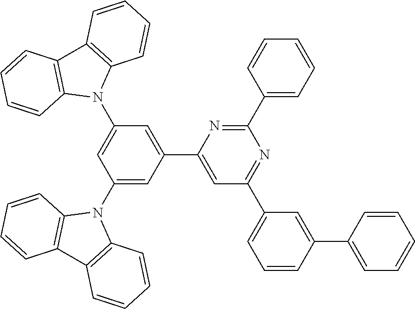 [SMILES: c1ccc(-c2cccc(-c3cc(-c4cc(-n5c6ccccc6c6ccccc65)cc(-n5c6ccccc6c6ccccc65)c4)nc(-c4ccccc4)n3)c2)cc1] | Compound ET1 |
| 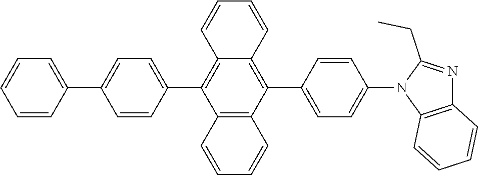 [SMILES: CCc1nc2ccccc2n1-c1ccc(-c2c3ccccc3c(-c3ccc(-c4ccccc4)cc3)c3ccccc23)cc1] | Compound ET2 |
| 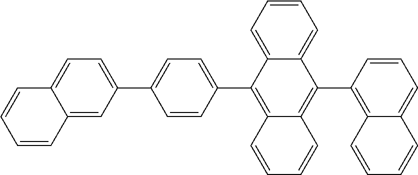 [SMILES: c1ccc2cc(-c3ccc(-c4c5ccccc5c(-c5cccc6ccccc56)c5ccccc45)cc3)ccc2c1] | Compound BH1 |

### Application Examples 1-4, Comparative Application Example 2

Comparative Application Example 1 was repeated except for using the Dopant compounds shown in place of the Comparative Compound 1. The device results are shown in Table 1 and Table 2.

TABLE 1

| Application | | | |
|---|---|---|---|
| Example | | λₘₐₓ/nm | LT-90/h |
| Dopant compound | Dopant compound | @ 10 mA/cm² | @ 50 mA/cm² |
| Comp. Appl. Ex. 2 | 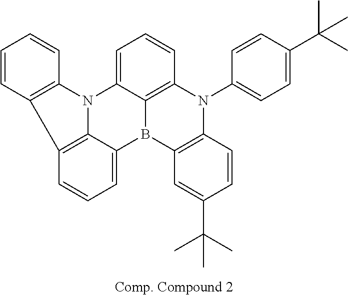 [SMILES: CC(C)(C)c1ccc(N2c3ccc(C(C)(C)C)cc3B3c4c2cccc4-n2c4ccccc4c4cccc3c42)cc1] | 477 | 151 |
| Appl. Ex. 2 | 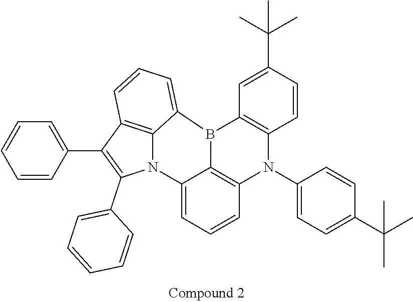 [SMILES: CC(C)(C)c1ccc(N2c3ccc(C(C)(C)C)cc3B3c4c2cccc4-n2c(-c4ccccc4)c(-c4ccccc4)c4cccc3c42)cc1] | 480 | 290 |
| Appl. Ex. 3 | 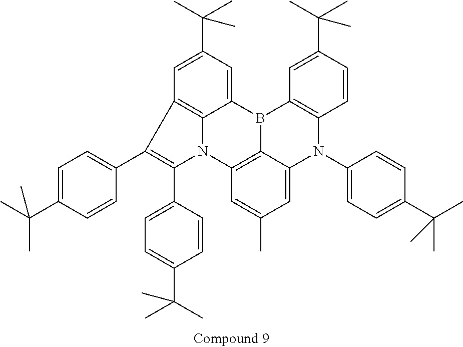 [SMILES: Cc1cc2c3c(c1)-n1c(-c4ccc(C(C)(C)C)cc4)c(-c4ccc(C(C)(C)C)cc4)c4cc(C(C)(C)C)cc(c41)B3c1cc(C(C)(C)C)ccc1N2c1ccc(C(C)(C)C)cc1] | 479 | 261 |
| Appl. Ex. 1 | 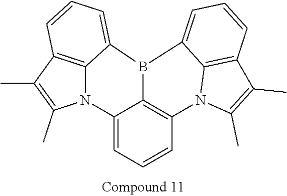 | 489 | 188 |

TABLE 2

| Application Example | | λₘₐₓ/nm | LT-90/h |
|---|---|---|---|
| Dopant compound | Dopant compound | @ 10 mA/cm² | @ 50 mA/cm² |
| Comp. Appl. Ex. 1 | 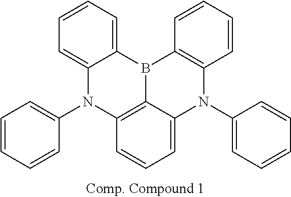 [SMILES: c1ccc(N2c3ccccc3B3c4ccccc4N(c4ccccc4)c4cccc2c43)cc1] | 459 | 63 |
| Appl. Ex. 4 | 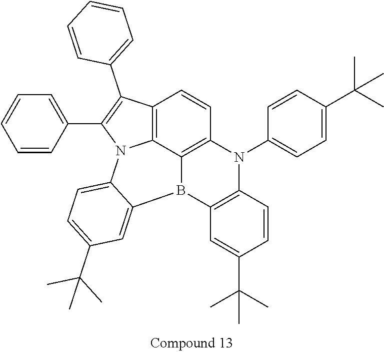 [SMILES: CC(C)(C)c1ccc(N2c3ccc(C(C)(C)C)cc3B3c4cc(C(C)(C)C)ccc4-n4c(-c5ccccc5)c(-c5ccccc5)c5ccc2c3c54)cc1] | 454 | 98 |

The results shown in Table 1 and Table 2 demonstrated that the lifetime was improved in the case when inventive compounds were used as a dopant together with the host compound BH1 in an OLED.

### Application Example 5, Comparative Application Example 3

Comparative Application Example 1 was repeated except for using 2% by weight of the Comparative Compound 2 (Comparative Application Example 3) respectively 2% by weight of the inventive Compound 5 (Application Example 5) as a dopant. The device results are shown in Table 3.

To characterize the OLED, electroluminescence spectra were recorded at various currents and voltages. In addition, the current-voltage characteristic was measured in combination with the luminance to determine luminous efficiency and external quantum efficiency (EQE). 90% lifetime (LT90), the time spent until the initial luminance at 50 mA/cm² was reduced to 90%, was recorded.

TABLE 3

| Application | | | | |
|---|---|---|---|---|
| Example | | | | |
| Dopant | | EQE/% | λₘₐₓ/nm | LT-90/h |
| compound | Dopant compound | @ 10 mA/cm² | @ 10 mA/cm² | @ 50 mA/cm² |
| Comp. Appl. Ex. 3 | 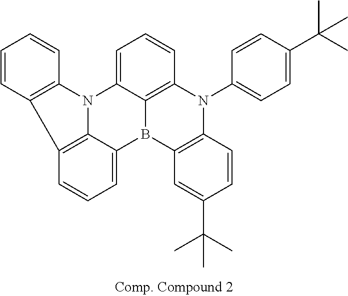 [SMILES: CC(C)(C)c1ccc(N2c3ccc(C(C)(C)C)cc3B3c4c2cccc4-n2c4ccccc4c4cccc3c42)cc1] | 6.26 | 477 | 92.2 |
| Appl. Ex. 5 | 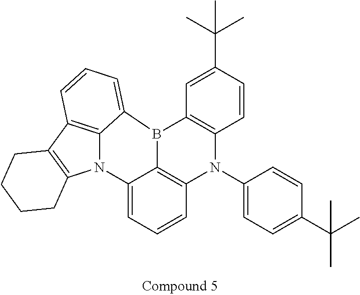 [SMILES: CC(C)(C)c1ccc(N2c3ccc(C(C)(C)C)cc3B3c4c2cccc4-n2c4c(c5cccc3c52)CCCC4)cc1] | 7.43 | 474 | 106.6 |

The results shown in Table 3 demonstrated that the lifetime and the EQE were improved in the case when inventive compounds were used as a dopant together with the host compound BH1 in an OLED.

### Application Examples 6-15, Comparative Application Example 4

Comparative Application Example 1 was repeated except for using 2% by weight of the Comparative Compound 1 (Comparative Application Example 4) respectively 2% by weight of the inventive Compound used as a dopant. The device results are shown in Table 4.

To characterize the OLED, the current-voltage characteristic was measured in combination with the luminance to determine luminous efficiency and external quantum efficiency (EQE). 95% lifetime (LT95), the time spent until the initial luminance at 50 mA/cm² was reduced to 95%, was recorded.

TABLE 4

| Application | | | | |
|---|---|---|---|---|
| Example | | | | |
| Dopant | | EQE/% | λₘₐₓ/nm | LT-95/h |
| compound | Dopant compound | @ 10 mA/cm² | @ 10 mA/cm² | @ 50 mA/cm² |
| Comp. Appl. Ex. 4 | 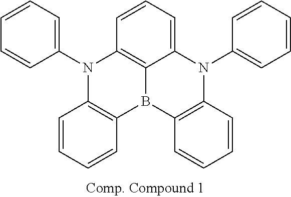 [SMILES: c1ccc(N2c3ccccc3B3c4ccccc4N(c4ccccc4)c4cccc2c43)cc1] | 7.1 | 459 | 45 |
| Appl. Ex. 6 | 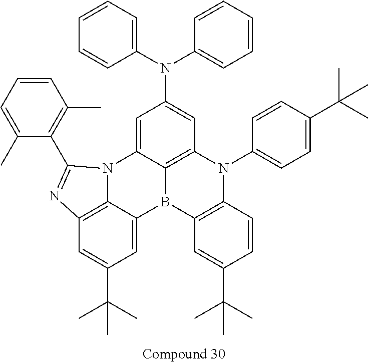 [SMILES: Cc1cccc(C)c1-c1nc2cc(C(C)(C)C)cc3c2n1-c1cc(N(c2ccccc2)c2ccccc2)cc2c1B3c1cc(C(C)(C)C)ccc1N2c1ccc(C(C)(C)C)cc1] | 9.2 | 458 | 107 |
| Appl. Ex. 7 | 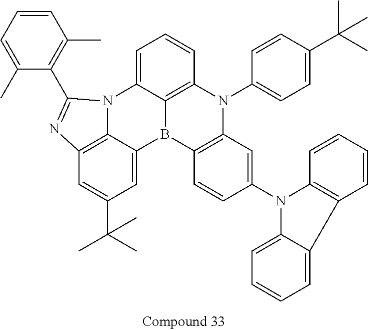 [SMILES: Cc1cccc(C)c1-c1nc2cc(C(C)(C)C)cc3c2n1-c1cccc2c1B3c1ccc(-n3c4ccccc4c4ccccc43)cc1N2c1ccc(C(C)(C)C)cc1] | 9.8 | 459 | 82 |
| Appl. Ex. 8 | 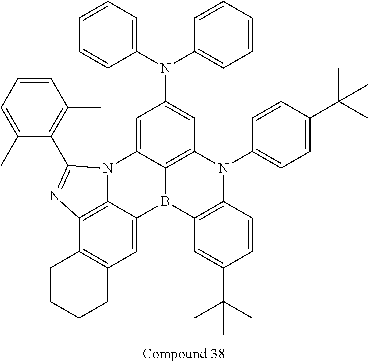 [SMILES: Cc1cccc(C)c1-c1nc2c3c(cc4c2n1-c1cc(N(c2ccccc2)c2ccccc2)cc2c1B4c1cc(C(C)(C)C)ccc1N2c1ccc(C(C)(C)C)cc1)CCCC3] | 9.2 | 457 | 73 |
| Appl. Ex. 9 | 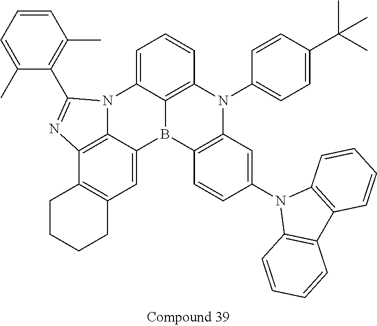 [SMILES: Cc1cccc(C)c1-c1nc2c3c(cc4c2n1-c1cccc2c1B4c1ccc(-n4c5ccccc5c5ccccc54)cc1N2c1ccc(C(C)(C)C)cc1)CCCC3] | 9.4 | 459 | 59 |
| Appl. Ex. 10 | 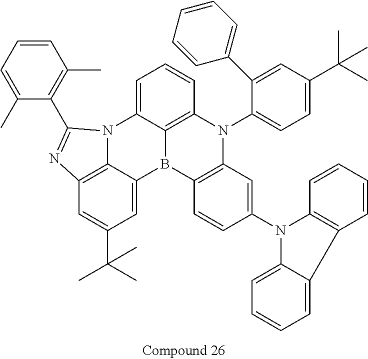 [SMILES: Cc1cccc(C)c1-c1nc2cc(C(C)(C)C)cc3c2n1-c1cccc2c1B3c1ccc(-n3c4ccccc4c4ccccc43)cc1N2c1ccc(C(C)(C)C)cc1-c1ccccc1] | 9.6 | 458 | 106 |
| Appl. Ex. 11 | 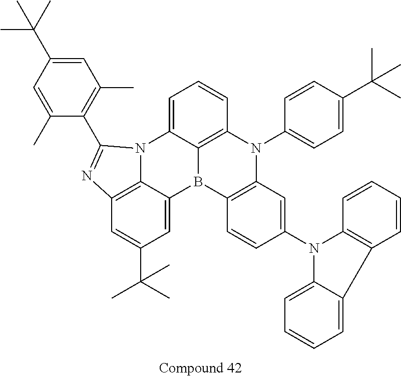 [SMILES: Cc1cc(C(C)(C)C)cc(C)c1-c1nc2cc(C(C)(C)C)cc3c2n1-c1cccc2c1B3c1ccc(-n3c4ccccc4c4ccccc43)cc1N2c1ccc(C(C)(C)C)cc1] | 10.0 | 459 | 92 |
| Appl. Ex. 12 | 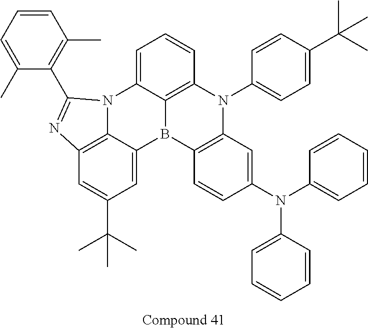 [SMILES: Cc1cccc(C)c1-c1nc2cc(C(C)(C)C)cc3c2n1-c1cccc2c1B3c1ccc(N(c3ccccc3)c3ccccc3)cc1N2c1ccc(C(C)(C)C)cc1] | 9.2 | 456 | 73 |
| Appl. Ex. 13 | 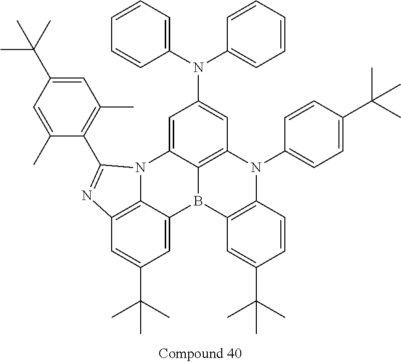 [SMILES: Cc1cc(C(C)(C)C)cc(C)c1-c1nc2cc(C(C)(C)C)cc3c2n1-c1cc(N(c2ccccc2)c2ccccc2)cc2c1B3c1cc(C(C)(C)C)ccc1N2c1ccc(C(C)(C)C)cc1] | 9.7 | 458 | 104 |
| Appl. Ex. 14 | 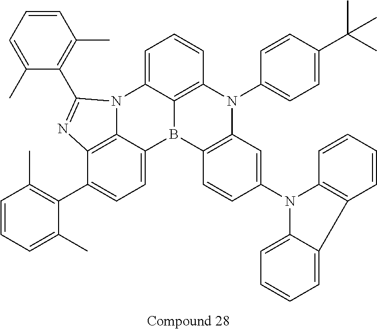 [SMILES: Cc1cccc(C)c1-c1ccc2c3c1nc(-c1c(C)cccc1C)n3-c1cccc3c1B2c1ccc(-n2c4ccccc4c4ccccc42)cc1N3c1ccc(C(C)(C)C)cc1] | 10.0 | 460 | 60 |
| Appl. Ex. 15 | 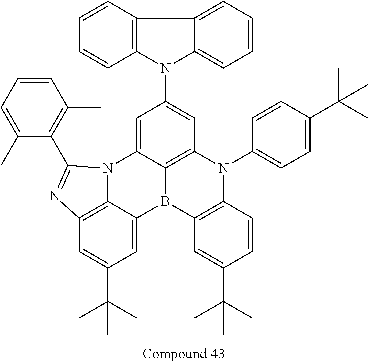 [SMILES: Cc1cccc(C)c1-c1nc2cc(C(C)(C)C)cc3c2n1-c1cc(-n2c4ccccc4c4ccccc42)cc2c1B3c1cc(C(C)(C)C)ccc1N2c1ccc(C(C)(C)C)cc1] | 9.4 | 458 | 93 |

The results shown in Table 4 demonstrated that the lifetime and the EQE were improved in the case when inventive compounds were used as a dopant together with the host compound BH1 in an OLED.

1: A polycyclic compound represented by formula (I): 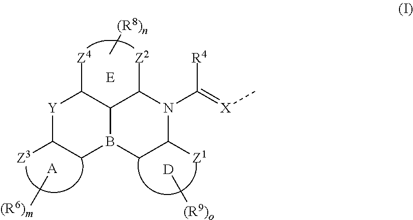
wherein
ring A, ring E and ring D each independently represents an aromatic group having 6 to 30 ring carbon atoms or a heteroaromatic group having 3 to 30 ring atoms;
X represents CR⁵ or N; [symbol]
the dotted line represents a single bond connected with Z¹ or connected with Z²;
Z¹ represents C in the case that it is connected with the dotted line at X, and Z¹ represents CR^{X9} or N in the case that it is not connected with the dotted line at X;
Z² represents C in the case that it is connected with the dotted line at X, and Z² represents CR^{X8} or N in the case that it is not connected with the dotted line at X;
Y represents NR¹, O, S, 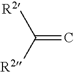
CR²₂;
R⁴ and R⁵ each independently represents H, halogen, a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, a substituted or unsubstituted aryloxy group having 6 to 24 ring carbon atoms, a substituted or unsubstituted alkylthio group having 1 to 25 carbon atoms, a substituted or unsubstituted arylthio group having 6 to 24 ring carbon atoms, an alkyl and/or aryl substituted silyl group, an alkyl or aryl substituted carbonyl group, an alkyl, aryl or heteroaryl substituted amino group, an alkyl or aryl substituted amide group, an alkyl or aryl substituted carboxyl group, a substituted phosphoryl group, CN, or a substituted or unsubstituted fluoroalkyl group having 1 to 25 carbon atoms; or
R⁴ and R⁵ may form together an unsubstituted or substituted aliphatic ring;
R¹ represents a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms or a substituted or unsubstituted fluoroalkyl group having 1 to 25 carbon atoms; or
a group of formula 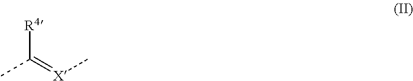
wherein X′ represents CR^{5′} or N; R^{4′}, R^{5′} and X′ are defined as R⁴, R⁵ and X;
with the difference that R^{4′} and R^{5′} may form together an unsubstituted or substituted ring; [symbol]
the dotted line at X′ represents a single bond connected with Z³ or connected with Z⁴;
Z³ represents C in the case that it is connected with the dotted line at X′, and Z³ represents CR^{X6A} or N in the case that it is not connected with the dotted line at X′;
Z⁴ represents C in the case that it is connected with the dotted line at X′, and Z⁴ represents CR^{X8A} or N in the case that it is not connected with the dotted line at X′; [symbol]
the other dotted line in the group of formula (II) represents a bonding site to the N atom of the group NR¹;
wherein R¹ may be connected to ring A or ring E;
R², R^{2′} and R^{2″} each independently represents H, halogen, a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, a substituted or unsubstituted aryloxy group having 6 to 24 ring carbon atoms, a substituted or unsubstituted alkylthio group having 1 to 25 carbon atoms, a substituted or unsubstituted arylthio group having 6 to 24 ring carbon atoms, an alkyl and/or aryl substituted silyl group, an alkyl or aryl substituted carbonyl group, an alkyl, aryl or heteroaryl substituted amino group, an alkyl or aryl substituted amide group, an alkyl or aryl substituted carboxyl group, a substituted phosphoryl group, CN, or a substituted or unsubstituted fluoroalkyl group having 1 to 25 carbon atoms, or
two residues R² may form together an unsubstituted or substituted ring, or;
one of the residues R^{2′} or R^{2″} may be connected with ring A or ring E;
R⁶, R⁸, R⁹, R^{X6A}, R^{X8A}, R^{X8} and R^{X9}, each independently represents H, halogen, a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, a substituted or unsubstituted aryloxy group having 6 to 24 ring carbon atoms, a substituted or unsubstituted alkylthio group having 1 to 25 carbon atoms, a substituted or unsubstituted arylthio group having 6 to 24 ring carbon atoms, an alkyl and/or aryl substituted silyl group, an alkyl or aryl substituted carbonyl group, an alkyl or aryl substituted carboxyl group, alkyl or aryl substituted amide group, an alkyl, aryl or heteroaryl substituted amino group, a substituted phosphoryl group, CN, or a substituted or unsubstituted fluoroalkyl group having 1 to 25 carbon atoms;
wherein two adjacent groups R⁶, two adjacent groups R⁸ and/or two adjacent groups R⁹ and/or R^{X6A} and a group R⁶ adjacent to R^{X6A} and/or R^{X9} and a group R⁹ adjacent to R^{X9} and/or R^{X8} and a group R⁸ adjacent to R^{X8} and/or R^{X8A} and a group R⁸ adjacent to R^{X8A}, may form together an unsubstituted or substituted ring;
n is 0 or 1; and
m and o are each independently 0, 1, 2 or 3.

2: The compound according to claim 1, which is represented by formula (III) 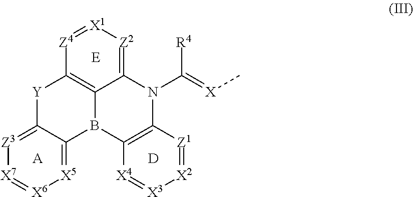
wherein
X¹ is CR^{X1} or N;
X² is CR^{X2} or N;
X³ is CR^{X3} or N;
X⁴ is CR^{X4} or N;
X⁵ is CR^{X5} or N;
X⁶ is CR^{X6} or N;
X⁷ is CR^{X7} or N; and
R^{X1}, R^{X2}, R^{X3}, R^{X4}, R^{X5}, R^{X6} and R^{X7} each independently represents H, halogen, a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, a substituted or unsubstituted aryloxy group having 6 to 24 ring carbon atoms, a substituted or unsubstituted alkylthio group having 1 to 25 carbon atoms, a substituted or unsubstituted arylthio group having 6 to 24 ring carbon atoms, an alkyl and/or aryl substituted silyl group, an alkyl or aryl substituted carbonyl group, an alkyl or aryl substituted carboxyl group, alkyl or aryl substituted amide group, an alkyl, aryl or heteroaryl substituted amino group, a substituted phosphoryl group, CN, or a substituted or unsubstituted fluoroalkyl group having 1 to 25 carbon atoms,
wherein two adjacent groups R^{X5} and R^{X6}, R^{X6} and RX and/or RX and R^{X6A}, two adjacent groups R^{X8} and R^{X1} and/or R^{X1} and R^{X8A}, and/or two adjacent groups R^{X4} and R^{X3}, R^{X3} and R^{X2}, and/or R^{X2} and R^{X9} may form together an unsubstituted or substituted ring.

3: The compound according to claim 2, which is represented by formula (IV)

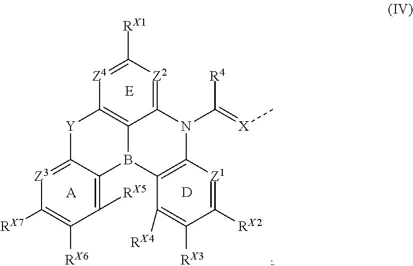

4: The compound according to claim 3, which is represented by formulae (V) and (VI)

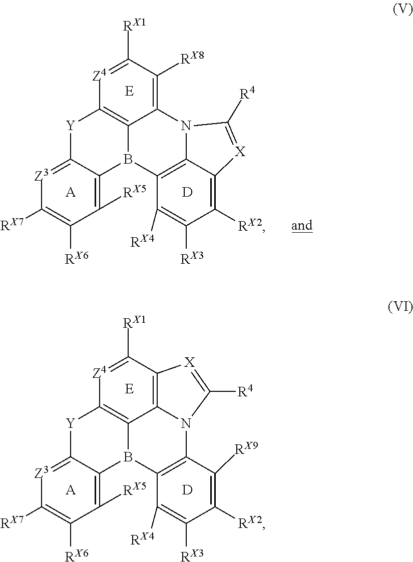

5: The compound according to claim 4, which is represented by formulae (VII), (VIII), (IX), (X) and (XI) 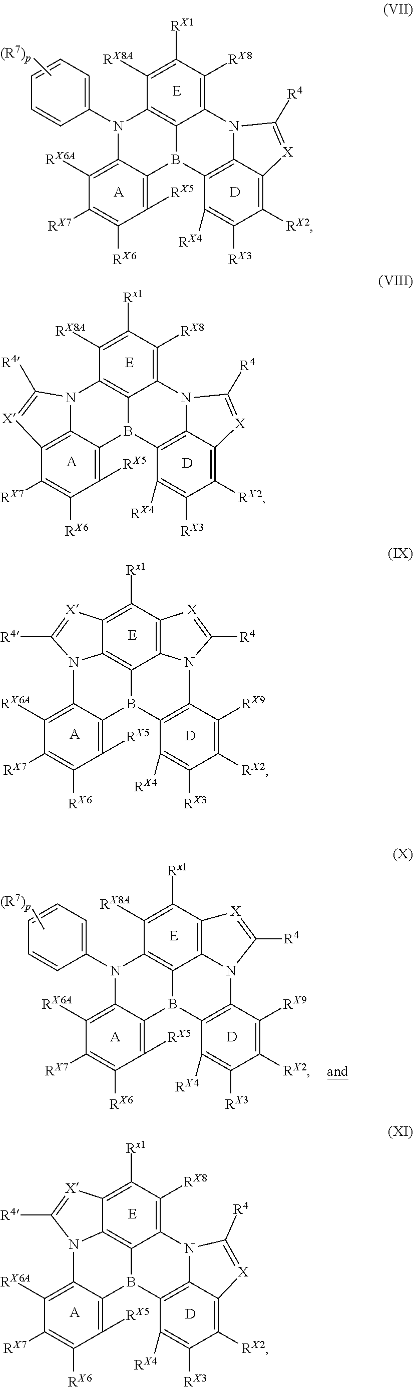
wherein
R⁷ represents H, halogen, a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, a substituted or unsubstituted aryloxy group having 6 to 24 ring carbon atoms, a substituted or unsubstituted alkylthio group having 1 to 25 carbon atoms, a substituted or unsubstituted arylthio group having 6 to 24 ring carbon atoms, an alkyl and/or aryl substituted silyl group, an alkyl or aryl substituted carbonyl group, an alkyl or aryl substituted carboxyl group, alkyl or aryl substituted amide group, an alkyl, aryl or heteroaryl substituted amino group, a substituted phosphoryl group, CN, or a substituted or unsubstituted fluoroalkyl group having 1 to 25 carbon atoms;
wherein two adjacent groups R⁷ may form together an unsubstituted or substituted ring
or
R⁷ and R^{X6A} and/or R⁷ and R^{X8A} may form together an unsubstituted or substituted ring; and
p represents, 0, 1, 2, 3, 4 or 5.

6: The compound according to claim 5, which is represented by formulae (XII), (XIII), (XIV), (XV), (XVI), (XVII), (XVIII), (XIX), (XX), (XXI), (XXII), (XXIII), (XXIV) and (XXV)

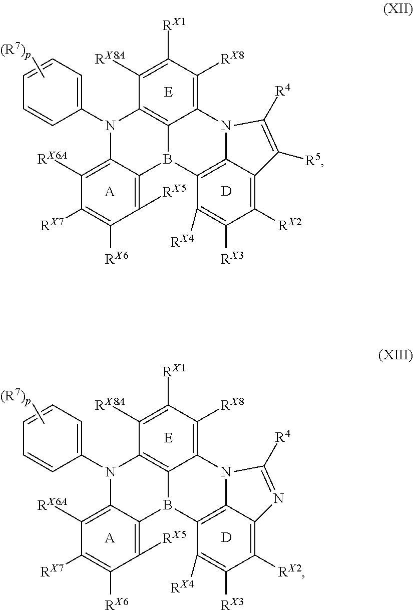

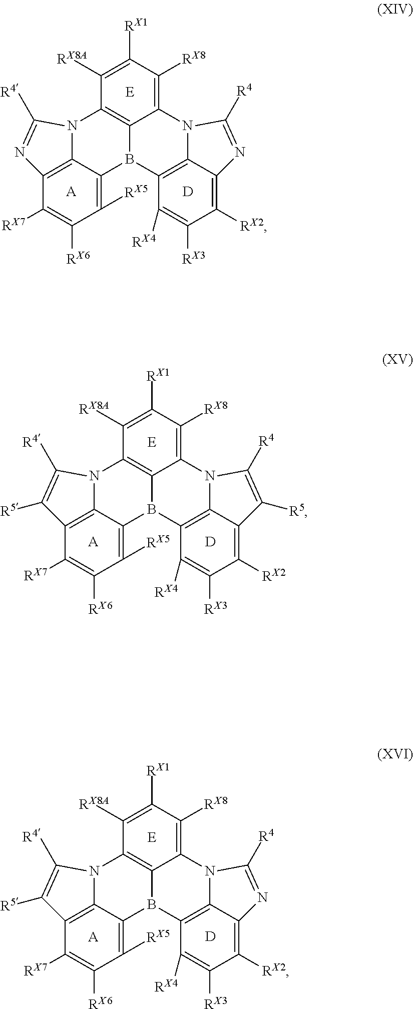

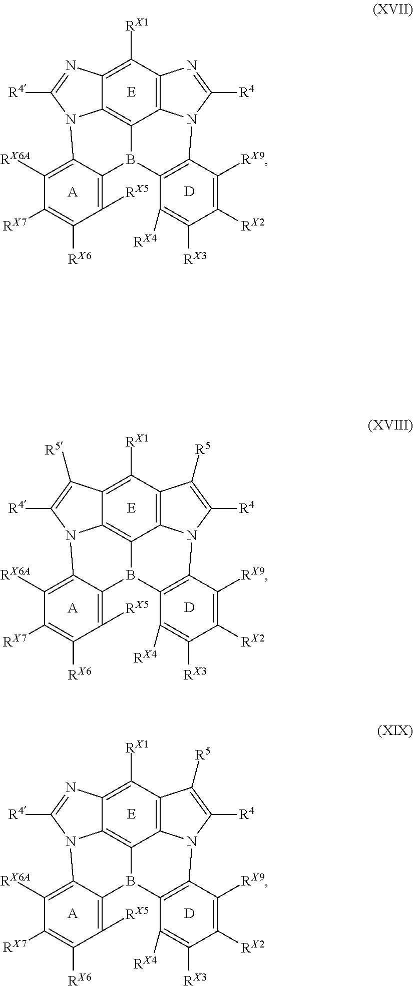

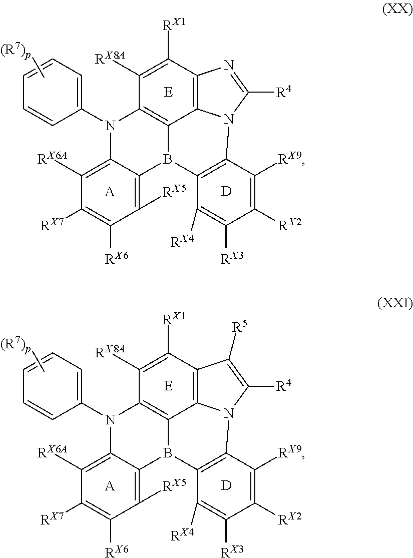

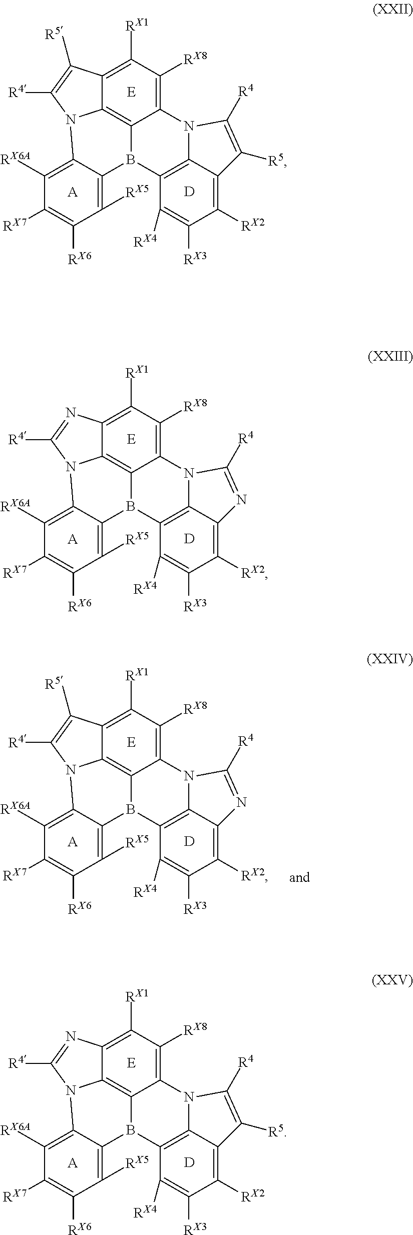

7: The compound according to claim 1, wherein
R⁴, R⁵, R^{4′} and R^{5′} each independently represents H, a substituted or unsubstituted alkyl group having 1 to 25 carbon atoms, a substituted or unsubstituted aryl group having 6 to 30 ring carbon atoms, a substituted or unsubstituted fluoroalkyl group having 1 to 25 carbon atoms, a substituted or unsubstituted heteroaryl group having 5 to 18 ring atoms, an alkyl and/or aryl substituted silyl group, or an alkyl, aryl or heteroaryl substituted amino group;
or
R and R⁵ together form a substituted or unsubstituted cyclohexyl ring;
and/or
R^{4′} and R^{5′} together form a substituted or unsubstituted phenyl ring or a substituted or unsubstituted cyclohexyl ring.

8: The compound according to claim 1, wherein
R^{X1}, R^{X8}, R⁶, R⁷, R⁸, R⁹, R^{X2}, R^{X3}, R^{X4}, R^{X5}, R^{X6}, R^{X7}, R^{X6A}, R^{X8A} and R^{X9} each independently represents H, a substituted or unsubstituted alkyl group having 1 to 25 carbon atoms; a substituted or unsubstituted aryl group having 6 to 30 ring carbon atoms, a substituted or unsubstituted fluoroalkyl group having 1 to 25 carbon atoms, a substituted or unsubstituted heteroaryl group having 5 to 18 ring atoms, an alkyl and/or aryl substituted silyl group, an alkyl, aryl or heteroaryl substituted amino group, a substituted or unsubstituted alkoxy group having 1 to 25 carbon atoms, or a substituted or unsubstituted aryloxy group having 1 to 25 ring carbon atoms;
wherein two adjacent groups R^{X5} and R^{X6}, R^{X6} and R^{X7} and/or R^{X7} and R^{X6A}, two adjacent groups R^{X}S and RXI and/or RXI and R^{X}BA, and/or two adjacent groups R^{X4} and R^{X3}, R^{X3} and R^{X2}, and/or R² and R^{X9} may form together an unsubstituted or substituted ring;
and/or
R⁷ and R^{X6A} and/or R⁷ and R^{X8A} may form together a ring by one of the following bridging groups: single bond, —CR¹⁰₂—, —NR¹¹—, or —C(R²)═C(R¹³)—; and/or
two adjacent groups R⁷ may form together an unsubstituted or substituted ring;
wherein
R¹⁰ represents H or a substituted or unsubstituted alkyl group having 1 to 8 carbon atoms;
R¹¹ represents a substituted or unsubstituted aryl group having 6 to 30 ring carbon atoms or a substituted or unsubstituted heteroaryl group having 5 to 18 ring atoms; and
R¹² and R¹³ each independently represents H, a substituted or unsubstituted alkyl group having 1 to 8 carbon atoms, a substituted or unsubstituted aryl group having 6 to 18 ring carbon atoms or a substituted or unsubstituted heteroaryl group having 5 to 14 ring atoms; or
R¹² and R¹³ together form a substituted or unsubstituted carbocyclic or heterocyclic ring comprising 5 or 6 ring atoms;
R^{X1} and R^{X8} each independently represents H, a substituted or unsubstituted alkyl group having 1 to 8 carbon atoms; or a substituted or unsubstituted phenyl group, unsubstituted or substituted biphenyl; or phenyl substituted by halogen;
a substituted or unsubstituted phenyloxy group, a substituted or unsubstituted diarylamino group, or a substituted or unsubstituted carbazolyl group linked via N;
or
R⁷ and R^{X6A} and/or R⁷ and R^{X8A} may form together a ring by one of the following bridging groups: single bond, —CR¹⁰₂—, —NR¹¹—, or —C(R¹²)═C(R¹³);
wherein
R¹⁰ represents H, methyl, ethyl, iso-propyl, n-propyl, n-butyl, iso-butyl, sec-butyl, n-pentyl, sec-pentyl, 3-pentyl, 2-methylbutyl, 3-methylbut-2-yl, 2-methylbut-2-yl or 2,2-dimethylpropyl;
R¹¹ represents a substituted or unsubstituted phenyl group or a substituted or unsubstituted heteroaryl group having 5 to 10 ring atoms; and
R¹² and R¹³ each independently represents H, methyl, ethyl, iso-propyl, n-propyl, n-butyl, iso-butyl, sec-butyl, n-pentyl, sec-pentyl, 3-pentyl, 2-methylbutyl, 3-methylbut-2-yl, 2-methylbut-2-yl or 2,2-dimethylpropyl, a substituted or unsubstituted phenyl group or a substituted or unsubstituted heteroaryl group having 5 to 10 ring atoms; or
R¹² and R¹³ together form a substituted or unsubstituted aromatic ring comprising 6 ring atoms.

9: A material for an organic electroluminescence device, comprising at least one compound according to claim 1.

10: An organic electroluminescence device comprising a cathode, an anode, and one or more organic thin film layers comprising an emitting layer disposed between the cathode and the anode, wherein at least one layer of the organic thin film layers comprises at least one compound according to claim 1.

11: The organic electroluminescence device according to claim 10, wherein the light emitting layer comprises the at least one compound.

12: The organic electroluminescence device according to claim 11, wherein the light emitting layer comprises at least one host and at least one dopant, wherein the dopant comprises the at least one compound.

13: The organic electroluminescence device according to claim 12, wherein the host comprises at least one substituted or unsubstituted anthracene compound.

14: The organic electroluminescence device according to claim 10, wherein an electron transporting layer is provided between the cathode and the light emitting layer, and the electron transporting layer comprises the at least one compound.

15: The organic electroluminescence device according to claim 10, wherein a hole blocking layer is provided between the electron transporting layer and the light emitting layer, and the hole blocking layer comprises the at least one compound.

16: An electronic equipment comprising the organic electroluminescence device according to claim 10.

17: A process for preparing the compound of formula (I) according to claim 1, the process comprising:
Borylation of a compound of formula (XXVI): 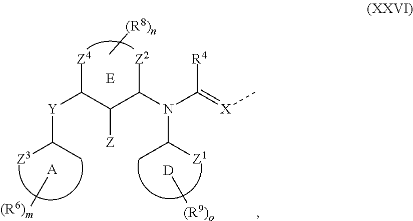
wherein
Q is halogen, or SiR¹⁴₃; and
R¹⁴ represents a substituted or unsubstituted alkyl group having 1 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, or a substituted or unsubstituted aryloxy group having 6 to 30 ring carbon atoms;
ring A, ring E and ring D each independently represents an aromatic group having 6 to 30 ring carbon atoms or a heteroaromatic group having 3 to 30 ring atoms;
X represents CR⁵ or N; [symbol]
the dotted line represents a single bond connected with Z¹ or connected with Z²;
Z¹ represents C in the case that it is connected with the dotted line at X, and Z¹ represents CR^{X9} or N in the case that it is not connected with the dotted line at X;
Z² represents C in the case that it is connected with the dotted line at X, and Z² represents CR^{X8} or N in the case that it is not connected with the dotted line at X;
Y represents NR¹, O, S, 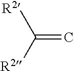
or CR²₂;
R⁴ and R⁵ each independently represents H, halogen, a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, a substituted or unsubstituted aryloxy group having 6 to 24 ring carbon atoms, a substituted or unsubstituted alkylthio group having 1 to 25 carbon atoms, a substituted or unsubstituted arylthio group having 6 to 24 ring carbon atoms, an alkyl and/or aryl substituted silyl group, an alkyl or aryl substituted carbonyl group, an alkyl, aryl or heteroaryl substituted amino group, an alkyl or aryl substituted amide group, an alkyl or aryl substituted carboxyl group, a substituted phosphoryl group, CN, or a substituted or unsubstituted fluoroalkyl group having 1 to 25 carbon atoms; or
R⁴ and R⁵ may form together an unsubstituted or substituted aliphatic ring;
R¹ represents a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, or a substituted or unsubstituted fluoroalkyl group having 1 to 25 carbon atoms; or
a group of formula 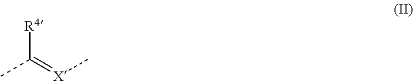
wherein X′ represents CR^{5′} or N; R^{4′}, R^{5′} and X′ are defined as R⁴, R⁵ and X;
with the difference that R^{4′} and R^{5′} may form together an unsubstituted or substituted ring; [symbol]
the dotted line at X′ represents a single bond connected with Z³ or connected with Z⁴;
Z³ represents C in the case that it is connected with the dotted line at X′, and Z³ represents CR^{X6A} or N in the case that it is not connected with the dotted line at X′;
Z⁴ represents C in the case that it is connected with the dotted line at X′, and Z⁴ represents CR^{X8A} or N in the case that it is not connected with the dotted line at X′; [symbol]
the other dotted line in the group of formula (II) represents a bonding site to the N atom of the group NR¹;
wherein R¹ may be connected to ring A or ring E;
R², R^{2′} and R^{2″} each independently represents H, halogen, a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, a substituted or unsubstituted aryloxy group having 6 to 24 ring carbon atoms, a substituted or unsubstituted alkylthio group having 1 to 25 carbon atoms, a substituted or unsubstituted arylthio group having 6 to 24 ring carbon atoms, an alkyl and/or aryl substituted silyl group, an alkyl or aryl substituted carbonyl group, an alkyl, aryl or heteroaryl substituted amino group, an alkyl or aryl substituted amide group, an alkyl or aryl substituted carboxyl group, a substituted phosphoryl group, CN; or a substituted or unsubstituted fluoroalkyl group having 1 to 25 carbon atoms, or
two residues R² may form together an unsubstituted or substituted ring, or;
one of the residues R^{2′} or R^{2″} may be connected with ring A or ring E;
R⁶, R⁸, R⁹, R^{X6A}, R^{X8A}, R^{X8} and R^{X9}, each independently represents H, halogen, a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, a substituted or unsubstituted aryloxy group having 6 to 24 ring carbon atoms, a substituted or unsubstituted alkylthio group having 1 to 25 carbon atoms, a substituted or unsubstituted arylthio group having 6 to 24 ring carbon atoms, an alkyl and/or aryl substituted silyl group, an alkyl or aryl substituted carbonyl group, an alkyl or aryl substituted carboxyl group, alkyl or aryl substituted amide group, an alkyl, aryl or heteroaryl substituted amino group, a substituted phosphoryl group, CN, or a substituted or unsubstituted fluoroalkyl group having 1 to 25 carbon atoms;
wherein two adjacent groups R⁶, two adjacent groups R⁸ and/or two adjacent groups R⁹ and/or R^{X6A} and a group R⁶ adjacent to R^{X6A} and/or R^{X9} and a group R⁹ adjacent to R^{X9} and/or R^{X8} and a group R⁸ adjacent to R^{X8} and/or R^{X8A} and a group R⁸ adjacent to R^{X8A}, may form together an unsubstituted or substituted ring;
n is 0 or 1; and
m and o are each independently 0, 1, 2 or 3.

18: The process according to claim 17 comprising:
ic) Transforming a compound of formula (XXVI) into a compound of formula (XXVII) 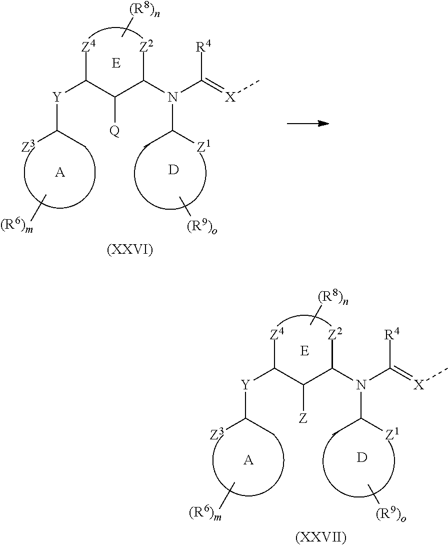
wherein
Z represents a B containing compound selected from the group consisting of B(R¹⁵)₂ and B(hal)₃⁻M⁺,
wherein
hal represents a halogen atom;
M represents an alkali metal, and
R¹⁵ represents halogen or OR¹⁶,
R¹⁶ represents H, an unsubstituted or substituted C₁ to C₁₈ alkyl group, or
two groups R¹⁶ may form together a ring; and 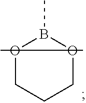
iic) Transforming the compound of formula (XXVII) to a compound of formula (I) 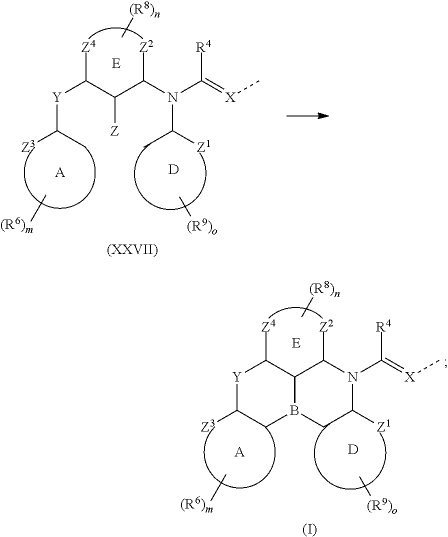
or
ib) Transforming a compound of formula (XXVI) into a compound of formula (XXVIII) and/or (XXIX) 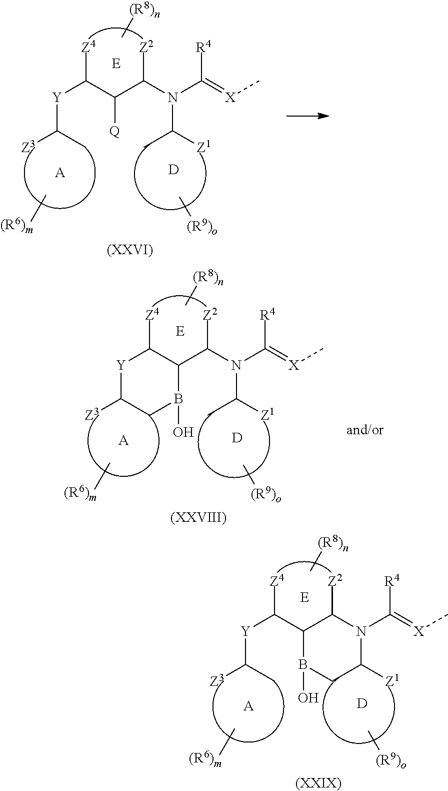
and
iib) Transforming the compound of formula (XXVIII) and/or (XXIX) to a compound of formula (I) 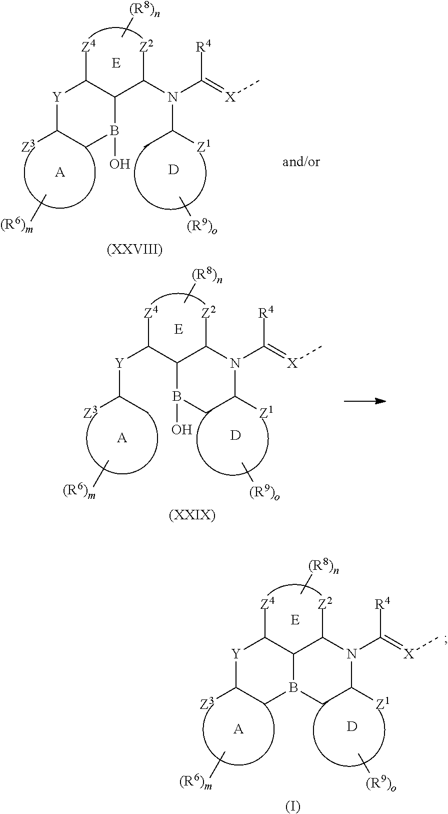
or
ic) Transforming a compound of formula (XXVI) into a compound of formula (XXVII) 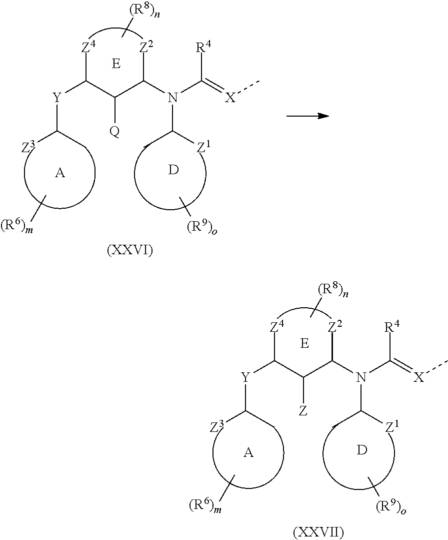
iic) Transforming a compound of formula (XXVII) into a compound of formula (XXVIII) and/or (XXIX) 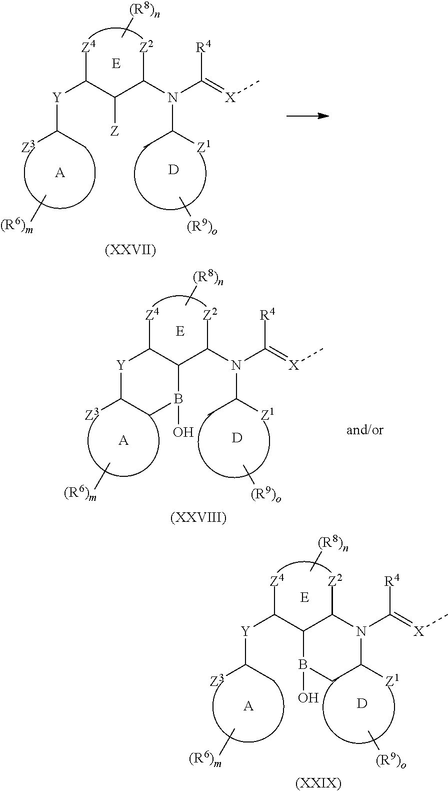
and
iiic) Transforming the compound of formula (XXVIII) and/or (XXIX) to the compound of formula (I) 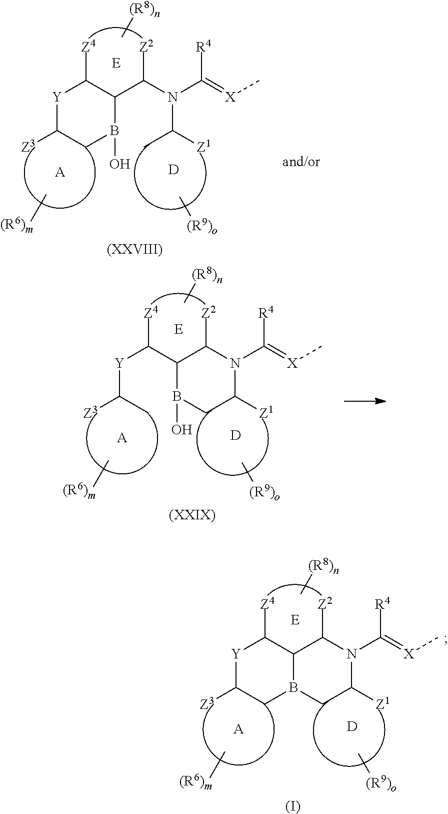
wherein
ring A, ring E and ring D each independently represents an aromatic group having 6 to 30 ring carbon atoms or a heteroaromatic group having 3 to 30 ring atoms;
X represents CR⁵ or N; [symbol]
the dotted line represents a single bond connected with Z¹ or connected with Z²;
Z¹ represents C in the case that it is connected with the dotted line at X, and Z¹ represents CR^{X9} or N in the case that it is not connected with the dotted line at X;
Z² represents C in the case that it is connected with the dotted line at X, and Z² represents CR^{X8} or N in the case that it is not connected with the dotted line at X;
Y represents NR¹, O, S, 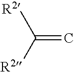
or CR²;
R⁴ and R⁵ each independently represents H, halogen, a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, a substituted or unsubstituted aryloxy group having 6 to 24 ring carbon atoms, a substituted or unsubstituted alkylthio group having 1 to 25 carbon atoms, a substituted or unsubstituted arylthio group having 6 to 24 ring carbon atoms, an alkyl and/or aryl substituted silyl group, an alkyl or aryl substituted carbonyl group, an alkyl, aryl or heteroaryl substituted amino group, an alkyl or aryl substituted amide group, an alkyl or aryl substituted carboxyl group, a substituted phosphoryl group, CN, or a substituted or unsubstituted fluoroalkyl group having 1 to 25 carbon atoms; or
R⁴ and R⁵ may form together an unsubstituted or substituted aliphatic ring;
R¹ represents a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, or a substituted or unsubstituted fluoroalkyl group having 1 to 25 carbon atoms; or
a group of formula 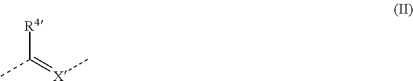
wherein X′ represents CR^{5′} or N; R^{4′}, R^{5′} and X′ are defined as R⁴, R⁵ and X;
with the difference that R⁴ and R⁵ may form together an unsubstituted or substituted ring; [symbol]
the dotted line at X′ represents a single bond connected with Z³ or connected with Z⁴;
Z³ represents C in the case that it is connected with the dotted line at X′, and Z³ represents CR^{X6A} or N in the case that it is not connected with the dotted line at X′;
Z⁴ represents C in the case that it is connected with the dotted line at X′, and Z⁴ represents CR^{X8A} or N in the case that it is not connected with the dotted line at X′; [symbol]
the other dotted line in the group of formula (II) represents a bonding site to the N atom of the group NR¹;
wherein R¹ may be connected to ring A or ring E;
R², R^{2′} and R^{2″} each independently represents H, halogen, a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, a substituted or unsubstituted aryloxy group having 6 to 24 ring carbon atoms, a substituted or unsubstituted alkylthio group having 1 to 25 carbon atoms, a substituted or unsubstituted arylthio group having 6 to 24 ring carbon atoms, an alkyl and/or aryl substituted silyl group, an alkyl or aryl substituted carbonyl group, an alkyl, aryl or heteroaryl substituted amino group, an alkyl or aryl substituted amide group, an alkyl or aryl substituted carboxyl group, a substituted phosphoryl group, CN, or a substituted or unsubstituted fluoroalkyl group having 1 to 25 carbon atoms, or
two residues R² may form together an unsubstituted or substituted ring, or;
one of the residues R^{2′} or R^{2″} may be connected with ring A or ring E;
R⁶, R⁸, R⁹, R^{X6A}, R^{X8A}, R^{X8} and R^{X9}, each independently represents H, halogen, a substituted or unsubstituted alkyl group having 1 to 25 carbon atoms, a substituted or unsubstituted alkenyl group having 2 to 25 carbon atoms, a substituted or unsubstituted alkynyl group having 2 to 25 carbon atoms, a substituted or unsubstituted cycloalkyl group having 3 to 25 ring carbon atoms, a substituted or unsubstituted alkoxy group having 1 to 25 carbon atoms, a substituted or unsubstituted aryl group having 6 to 30 ring carbon atoms, a substituted or unsubstituted heteroaryl group having 3 to 18 ring atoms, a substituted or unsubstituted heterocyclic group having 3 to 18 ring atoms, a substituted or unsubstituted aryloxy group having 6 to 24 ring carbon atoms, a substituted or unsubstituted alkylthio group having 1 to 25 carbon atoms, a substituted or unsubstituted arylthio group having 6 to 24 ring carbon atoms, an alkyl and/or aryl substituted silyl group, an alkyl or aryl substituted carbonyl group, an alkyl or aryl substituted carboxyl group, alkyl or aryl substituted amide group, an alkyl, aryl or heteroaryl substituted amino group, a substituted phosphoryl group, CN, or a substituted or unsubstituted fluoroalkyl group having 1 to 25 carbon atoms;
wherein two adjacent groups R⁶, two adjacent groups R⁸ and/or two adjacent groups R⁹ and/or R^{X6A} and a group R⁶ adjacent to R^{X6A} and/or R^{X9} and a group R⁹ adjacent to R^{X9} and/or R^{X8} and a group R⁸ adjacent to R^{X8} and/or R^{X8A} and a group R⁸ adjacent to R^{X8A}, may form together an unsubstituted or substituted ring;
n is 0 or 1; and
m and o are each independently 0, 1, 2 or 3.

19. (canceled)
